(19) United States
(12) Patent Application Publication
(10) Pub. No.: US 2023/0116972 A1
(43) Pub. Date: Apr. 20, 2023

(54) CD38 MODULATORS AND METHODS OF USE THEREOF

(71) Applicant: Cytokinetics, Inc., South San Francisco, CA (US)

(72) Inventors: Luke W. ASHCRAFT, San Francisco, CA (US); Chihyuan CHUANG, Millbrae, CA (US); Alfredo GARCIA, San Francisco, CA (US); Bradley P. MORGAN, Oakland, CA (US); Bartlomiej Przemyslaw IWAN, South San Francisco, CA (US); Molly Eichel MERMIN, South San Francisco, CA (US)

(21) Appl. No.: 17/811,790

(22) Filed: Jul. 11, 2022

Related U.S. Application Data
(60) Provisional application No. 63/203,190, filed on Jul. 12, 2021.

(51) Int. Cl. C07D 403/04; C07D 401/14; C07D 405/14

(52) U.S. Cl. CPC C07D 403/04; C07D 401/14; C07D 405/14

(57) ABSTRACT

Provided is a compound of formula (I):

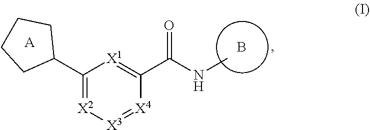

or a stereoisomer or tautomer thereof, or a pharmaceutically acceptable salt of any of the foregoing, wherein A, B, X¹, X², X³, and X⁴ are as defined herein.

Also provided is a pharmaceutically acceptable composition comprising a compound of formula (I), or a stereoisomer or tautomer thereof, or a pharmaceutically acceptable salt of any of the foregoing.

Also provided are methods of using a compound of formula (I), or a stereoisomer or tautomer thereof, or a pharmaceutically acceptable salt of any of the foregoing.

### CROSS REFERENCE TO RELATED APPLICATIONS

This application claims the priority benefit of U.S. Provisional Patent Application No. 63/203,190, filed Jul. 12, 2021, the disclosure of which is hereby incorporated herein by reference in its entirety.

### FIELD

Provided herein are compounds, pharmaceutical compositions comprising such compounds, and methods of treating various diseases, disorders, and conditions mediated by cluster of differentiation 38 (CD38) with such compounds and/or pharmaceutical compositions.

### BACKGROUND

The present disclosure relates to the use of modulators of CD38 and derivatives thereof, as well as inhibitors of CD38 expression, CD38 activity, or CD38-mediated signaling for preventing or treating a variety of pathological conditions.

Nicotinamide adenine dinucleotide (NAD+) is an essential coenzyme (enzyme cofactor) involved in fundamental biological processes of both catabolic and anabolic metabolism. As a coenzyme, NAD is associated with many oxidative enzymes (typically dehydrogenases) involved in energy metabolism, serving as a universal electron carrier. NAD exists in cells in the oxidized state (NAD+ and NADP+), and the reduced state (NADH and NADPH), acting as a chemical means to capture and transfer free energy from oxidative processes in catabolism, or to provide small packets of energy to build macromolecules in anabolism. NADH produced from the oxidation of carbohydrates, lipids, and amino acids provides reducing equivalents to the electron transport chain of mitochondria, ultimately driving the synthesis of ATP in oxidative phosphorylation.

More than 200 enzymes use either NAD+ or NADP+ as a coenzyme, and the enzymatic functions are not limited to energy metabolism. It is now appreciated that NAD+ plays a role in regulating diverse functions, including mitochondrial function, respiratory capacity, and biogenesis, mitochondrial-nuclear signaling. Further, it controls cell signaling, gene expression, DNA repair, hematopoiesis, immune function, the unfolded protein response, and autophagy. Furthermore, NAD is anti-inflammatory and is the precursor for NADPH, which is the primary source of reducing power for combating oxidative stress. A large body of literature indicates that boosting NAD levels is an effective strategy to either prevent or ameliorate a wide variety of disease states (Strømland et al., Biochem Soc Trans. 2019, 47(1):119-130; Ralto et al., Nat Rev Nephrol. 2019; Fang et al., Trends Mol Med. 2017, 23(10):899-916; Yoshino et al., Cell Metab. 2011, 14(4):528-36; Yang and Sauve, Biochim Biophys Acta. 2016, 1864:1787-1800; Verdin, Science. 2015, 350(6265):1208-13).

Levels of NAD+ and NADP+-associated enzymes play important roles in normal physiology and are altered under various disease and stress conditions including aging. Cellular NAD+ levels decrease during aging, metabolic disease, inflammatory diseases, during ischemia/reperfusion injury, and in other conditions in humans (Massudi et al., PLoS ONE. 2012, 7(7): e42357) and animals (Yang et al., Cell. 2007, 130(6):1095-107; Braidy et al. PLoS One. 2011, 26; 6 (4):e19194; Peek et al. Science. 2013, 342(6158):1243417; Ghosh et al., J Neurosci. 2012, 32(17):5821-32), suggesting that modulation of cellular NAD+ level affects the speed and severity of the decline and deterioration of bodily functions. Therefore, an increase in cellular NAD+ concentration could be beneficial in the context of aging and age-related diseases.

The cellular NAD+ pool is controlled by a balance between the activity of NAD+-synthesizing and consuming enzymes. In mammals, NAD+ is synthesized from a variety of dietary sources, including one or more of its major precursors that include: tryptophan (Trp), nicotinic acid (NA), nicotinamide riboside (NR), nicotinamide mononucleotide (NMN), and nicotinamide (NAM). Based upon the bioavailability of its precursors, there are three pathways for the synthesis of NAD+ in cells: (i) from Trp by the de novo biosynthesis pathway or kynurenine pathway (ii) from NA in the Preiss-Handler pathway and (iii) from NAM, NR, and NMN in the salvage pathway (Verdin et al., Science. 2015, 350(6265):1208-13). (Fulco et al, Dev Cell. 2008, 14(5):661-73; Imai, Curr Pharm Des. 2009, 15(1):20-8; Revollo et al., J Biol Chem. 2004, 279(49):50754-63; Revollo et al., Cell Metab. 2007, November; 6(5):363-75; van der Veer et al., J Biol Chem. 2007, 282(15):10841-5; Yang et al., Cell. 2007, 130(6):1095-107). Steady state levels of NAD+ can be depleted by a variety of NAD+-hydrolyzing enzymes including the sirtuin family of deacetylases, the DNA damage sensors poly (ADP-ribose) polymerases (PARPs), and NAD+ glycohydrolases including CD38 and CD157 (Canto et al, 2015, Yaku et al, 2018). CD38 is a multifunctional, type II transmembrane glycoprotein, expressed in cells of hematopoietic origin and non-lymphoid origin including non-parenchymal cells in skeletal and cardiac muscle. It is expressed primarily on the plasma membrane and also on the membranes on intracellular organelles. The primary catalytic reaction of CD38 involves the cleavage of a high energy β-glycoside bond between nicotinamide and the ribose moiety. CD38 is considered as the major NAD-consuming enzyme and plays a central role in NAD+ decline in mammals associated with aging, inflammation, senescence and various other stress-induced pathological conditions (Chini et al, 2018). Furthermore, CD38 mediates a selectin-like binding to endothelial cells, thus functioning as an adhesion molecule (Malavasi et al, 2008).

Thus, inhibiting CD38 catalysis by a small molecule would be an effective strategy to stabilize NAD levels and thereby address a broad spectrum of disease states. These include cardiac diseases, chemotherapy induced tissue damage, myocarditis, myocarditis associated with SARS-CoV-2 infection, immune-oncology, renal diseases, fibrotic diseases, metabolic diseases, muscular diseases, neurological diseases and injuries, diseases caused by impaired stem cell function, DNA damage and primary mitochondrial disorders, and ocular diseases.

### Summary

In one aspect, provided herein is a compound of formula (I):

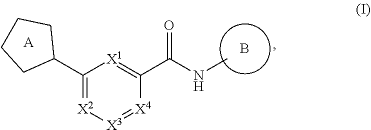

or a stereoisomer or tautomer thereof, or a pharmaceutically acceptable salt of any of the foregoing, wherein:

### X¹ is N or CH;

X² is N or C(R^{x}), wherein R^{x} is H, halo, or C₁₋₆alkyl;
X³ is N or C(R^{y}), wherein R^{y} is H, —OH, C₁₋₆alkoxy, C₃₋₁₀cycloalkyl, 3-10 membered heterocyclyl, or C₁₋₆ alkyl,
wherein the C₁₋₆ alkoxy of R^{y} is optionally substituted with one or more C₁₋₆alkoxy, the C₃₋₁₀cycloalkyl of R^{y} is optionally substituted with one or more halo, C₁₋₆alkoxy, or —OH, the 3-10 membered heterocyclyl of R^{y} is optionally substituted with one or more C₁₋₆alkyl, and the C₁₋₆alkyl of R^{y} is optionally substituted with one or more halo or —OH;
   X⁴ is N or C(R^{z}), wherein R^{z} is H, halo, —NH₂, C₁₋₆alkoxy, or C₁₋₆alkyl;
   provided that at most two of X¹, X², X³, and X⁴ are N;

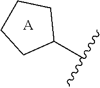

is:
(i)

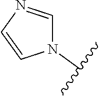 [SMILES: *n1ccnc1]

that is optionally substituted with one or more —C(O)—NH₂, or
(ii)

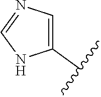 [SMILES: *c1cnc[nH]1]

that is optionally substituted with one or more C₁₋₆alkyl, or
(iii)

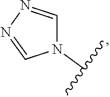 [SMILES: *n1cnnc1]

or
(iv)

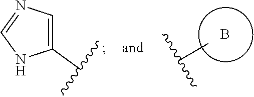

is:
(i) C₄₋₉cycloalkyl, wherein the C₄₋₉cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆ haloalkyl), phenyl, phenoxy, or pyridinyl,
wherein the C₁₋₆ alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
   (ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆alkyl, —C(O)—C₁₋₆ alkoxy, or phenyl, wherein the phenyl of R^{b} is optionally substituted with one or more C₁₋₆haloalkyl, or
   (iii) phenyl, wherein the phenyl is optionally substituted with one or more halo, or with C₁₋₆alkyl optionally substituted with —OH, or
   (iv) pyridinyl, wherein the pyridinyl is optionally substituted with one or more halo, C₁₋₆haloalkyl, C₁₋₆ alkoxy optionally substituted with one or more halo, C₁₋₆ alkyl optionally substituted with —OH, or —O—C₃₋₁₀cycloalkyl optionally substituted with one or more halo.

In one aspect, provided herein is a compound of formula (I):

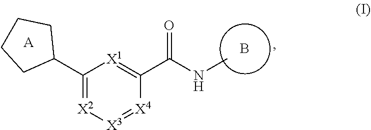

or a stereoisomer or tautomer thereof, or a pharmaceutically acceptable salt of any of the foregoing, wherein:

### X¹ is N or CH,

X² is N or C(R^{x}), wherein R^{x} is H, halo, or C₁₋₆alkyl,
X³ is N or C(R^{y}), wherein R^{y} is H, —OH, C₁₋₆alkoxy, C₃₋₁₀cycloalkyl, 3-10 membered heterocyclyl, or C₁₋₆alkyl, wherein the C₁₋₆alkyl of R^{y} is optionally substituted with one or more halo or —OH, and
X⁴ is N or C(R^{z}), wherein R^{z} is H, halo, or C₁₋₆alkyl, provided that at most two of X¹, X², X³, and X⁴ are N;

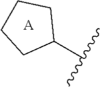

is:
(i)

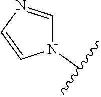 [SMILES: *n1ccnc1]

that is optionally substituted with one or more —C(O)—NH₂, or
(ii)

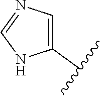 [SMILES: *c1cnc[nH]1]

that is optionally substituted with one or more C₁₋₆ alkyl, or
(iii)

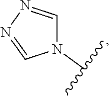 [SMILES: *n1cnnc1]

or
(iv)

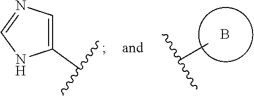

is:
(i) saturated C₄₋₈cycloalkyl, wherein the C₄₋₈cycloalkyl is optionally substituted with one or more R^{a}, wherein each R^{a} is independently —OH, halo, C₁₋₆alkyl, C₁₋₆haloalkyl, C₁₋₆alkoxy, or —C(O)—C₁₋₆ alkoxy, wherein the C₁₋₆alkoxy of R^{a} is optionally substituted with one or more C₁₋₆alkoxy and the C₁₋₆ alkyl of R^{a} is optionally substituted with one or more —OH or C₁₋₆alkoxy, or
(ii) saturated 4-8 membered heterocyclyl, wherein the 4-8 membered heterocyclyl is optionally substituted with one or more R^{b}, wherein each R^{b} is independently oxo, —C(O)—C₁₋₆alkyl, —C(O)—C₁₋₆ alkoxy, or phenyl, wherein the phenyl of R^{b} is optionally substituted with one or more C₁₋₆ haloalkyl, or
(iii) phenyl, wherein the phenyl is optionally substituted with one or more halo, or
(iv) pyridinyl, wherein the pyridinyl is optionally substituted with one or more halo, C₁₋₆alkyl, C₁₋₆haloalkyl, or C₁₋₆ alkoxy.

Also provided herein is a compound of formula (I-A1):

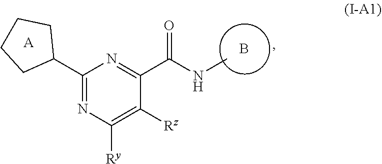

or a stereoisomer or tautomer thereof, or a pharmaceutically acceptable salt of any of the foregoing, wherein

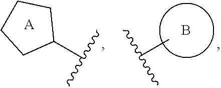

R^{y}, and R^{z} are as defined for a compound of formula (I).

Also provided herein is a compound of formula (I-A2):

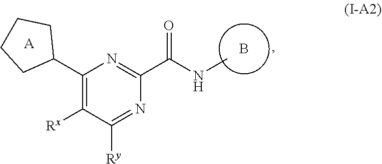

or a stereoisomer or tautomer thereof, or a pharmaceutically acceptable salt of any of the foregoing, wherein

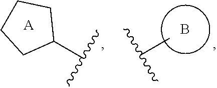

R^{x}, and R^{y} are as defined for a compound of formula (I).

Also provided herein is a compound of formula (I-A3):

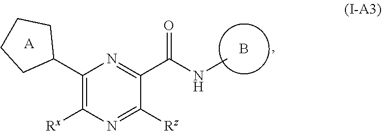

or a stereoisomer or tautomer thereof, or a pharmaceutically acceptable salt of any of the foregoing, wherein

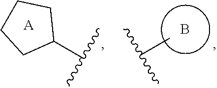

R^{x}, and R^{z} are as defined for a compound of formula (I).

Also provided herein is a compound of formula (I-B1):

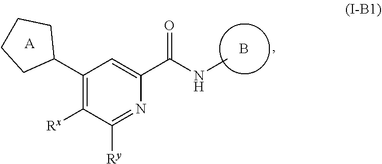

or a stereoisomer or tautomer thereof, or a pharmaceutically acceptable salt of any of the foregoing, wherein

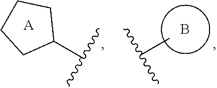

R^{x}, and R^{y} are as defined for a compound of formula (I).

Also provided herein is a compound of formula (I-B2):

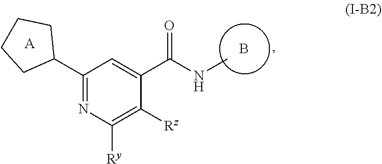

or a stereoisomer or tautomer thereof, or a pharmaceutically acceptable salt of any of the foregoing, wherein

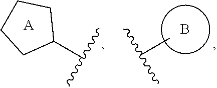

R^{y}, and R^{z} are as defined for a compound of formula (I).

Also provided herein is a compound of formula (I-B3):

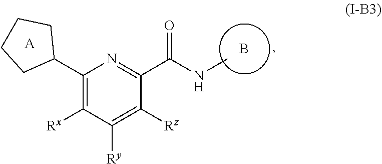

or a stereoisomer or tautomer thereof, or a pharmaceutically acceptable salt of any of the foregoing, wherein

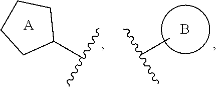

R^{y}, and R^{z} are as defined for a compound of formula (I).

Also provided herein is a compound of formula (I-C):

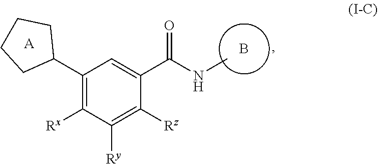

or a stereoisomer or tautomer thereof, or a pharmaceutically acceptable salt of any of the foregoing, wherein

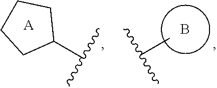

R^{x}, R^{y}, and R^{z} are as defined for a compound of formula (I).

Also provided herein is a compound of formula (I-D):

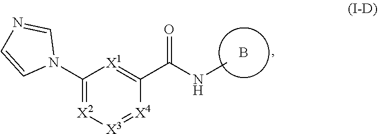

or a stereoisomer or tautomer thereof, or a pharmaceutically acceptable salt of any of the foregoing, wherein

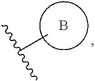

X¹, X², X³, and X⁴ are as defined for a compound of formula (I).

Also provided herein is a compound of formula (I-E):

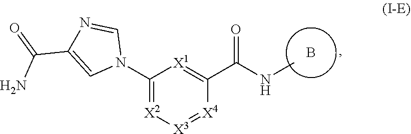

or a stereoisomer or tautomer thereof, or a pharmaceutically acceptable salt of any of the foregoing, wherein

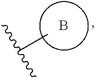

X¹, X², X³, and X⁴ are as defined for a compound of formula (I).

Also provided herein is a compound of formula (I-E):

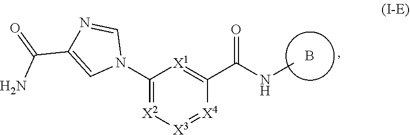

or a stereoisomer or tautomer thereof, or a pharmaceutically acceptable salt of any of the foregoing, wherein

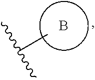

X¹, X², X³, and X⁴ are as defined for a compound of formula (I).

Also provided herein is a compound of formula (I-F):

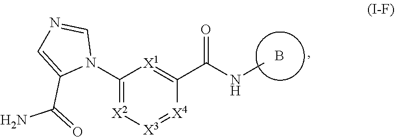

or a stereoisomer or tautomer thereof, or a pharmaceutically acceptable salt of any of the foregoing, wherein

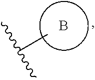

X¹, X², X³, and X⁴ are as defined for a compound of formula (I).

Also provided herein is a compound of formula (I-F):

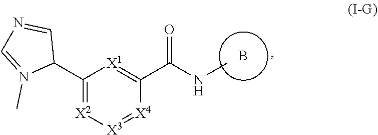

or a stereoisomer or tautomer thereof, or a pharmaceutically acceptable salt of any of the foregoing, wherein

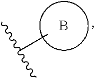

X¹, X², X³, and X⁴ are as defined for a compound of formula (I).

Also provided herein is a compound of formula (I-H):

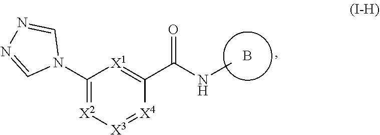

or a stereoisomer or tautomer thereof, or a pharmaceutically acceptable salt of any of the foregoing, wherein

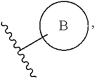

X¹, X², X³, and X⁴ are as defined for a compound of formula (I).

Also provided herein is a compound of formula (I-J):

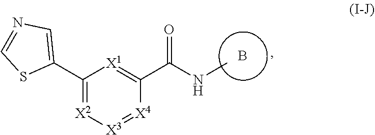

or a stereoisomer or tautomer thereof, or a pharmaceutically acceptable salt of any of the foregoing, wherein

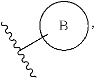

X¹, X², X³, and X⁴ are as defined for a compound of formula (I).

Also provided herein is a pharmaceutical composition, comprising: (i) an effective amount of a compound of formula (I), such as a compound of formula (I-A1), (I-A2), (I-A3), (I-B1), (I-B2), (I-B3), (I-C), (I-D), (I-E), (I-F), (I-G), (I-H), or (I-J), or a stereoisomer or tautomer thereof, or a pharmaceutically acceptable salt of any of the foregoing; and (ii) one or more pharmaceutically acceptable excipients.

Also provided herein is a method of treating a disease, disorder, or condition mediated by CD38 activity in a subject in need thereof, comprising administering to the subject (i) an effective amount of a compound of formula (I), such as a compound of formula (I-A1), (I-A2), (I-A3), (I-B1), (I-B2), (I-B3), (I-C), (I-D), (I-E), (I-F), (I-G), (I-H), or (I-J), or a stereoisomer or tautomer thereof, or a pharmaceutically acceptable salt of any of the foregoing, or (ii) a pharmaceutical composition comprising an effective amount of a compound of formula (I), such as a compound of formula (I-A1), (I-A2), (I-A3), (I-B1), (I-B2), (I-B3), (I-C), (I-D), (I-E), (I-F), (I-G), (I-H), or (I-J), or a stereoisomer or tautomer thereof, or a pharmaceutically acceptable salt of any of the foregoing, and one or more pharmaceutically acceptable excipients.

Additional embodiments, features, and advantages of the present disclosure will be apparent from the following detailed description and through practice of the present disclosure.

For the sake of brevity, the disclosures of publications cited in this specification, including patents, are herein incorporated by reference.

### BRIEF DESCRIPTION OF THE DRAWINGS

FIG. 1 shows tissue levels of nicotinamide in mouse tissues following administration of Compound 148 to aged C57BL/6 Mice.

### DETAILED DESCRIPTION

### Definitions

As used in the present specification, the following words and phrases are generally intended to have the meanings as set forth below, except to the extent that the context in which they are used indicates otherwise.

Throughout this application, unless the context indicates otherwise, references to a compound of formula (I) includes all subgroups of formula (I) defined herein, including all substructures, subgenera, preferences, embodiments, examples, and particular compounds defined and/or described herein. References to a compound of formula (I), and subgroups thereof, include ionic forms, polymorphs, pseudopolymorphs, amorphous forms, solvates, co-crystals, chelates, isomers, tautomers, oxides (e.g., N-oxides, S-oxides), esters, prodrugs, isotopes and/or protected forms thereof. In some embodiments, references to a compound of formula (I), and subgroups thereof, include polymorphs, solvates, co-crystals, isomers, tautomers, and/or oxides thereof. In some embodiments, references to a compound of formula (I), and subgroups thereof, include polymorphs, solvates, and/or co-crystals thereof. In some embodiments, references to a compound of formula (I), and subgroups thereof, include isomers, tautomers, and/or oxides thereof. In some embodiments, references to a compound of formula (I), and subgroups thereof, include solvates thereof. Similarly, the term “salts” includes solvates of salts of compounds.

“Alkyl” encompasses straight and branched carbon chains having the indicated number of carbon atoms, for example, from 1 to 20 carbon atoms, or 1 to 8 carbon atoms, or 1 to 6 carbon atoms. For example, C₁₋₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “propyl” includes n-propyl and isopropyl; and “butyl” includes n-butyl, sec-butyl, isobutyl and t-butyl. Examples of alkyl groups include, but are not limited to, methyl, ethyl, propyl, isopropyl, n-butyl, sec-butyl, tert-butyl, pentyl, 2-pentyl, 3-pentyl, isopentyl, neopentyl, hexyl, 2-hexyl, 3-hexyl, and 3-methylpentyl.

As used herein, the term “haloalkyl” refers to an alkyl moiety, as described above, wherein one or more of the hydrogen atoms of the alkyl moiety has been replaced by one or more independently selected halogen atoms. By way of illustration, the term “haloalkyl” includes, but it not limited to, a methyl moiety in which one or more of the hydrogen atoms of the methyl moiety has been replaced by one or more independently selected halogen atoms, e.g., —CH₂F, —CHF₂, —CH₂C₁, —CCl₃, —CHClF, —CCl₂Br, etc.

As used herein, the term “alkoxy” refers to a —O-alkyl moiety.

As used herein, the term “haloalkoxy” refers to an alkoxy moiety, as described above, wherein one or more of the hydrogen atoms of the alkoxy moiety has been replaced by one or more independently selected halogen atoms. By way of illustration, the term “haloalkoxy” includes, but it not limited to, a methoxy moiety in which one or more of the hydrogen atoms of the methoxy moiety has been replaced by one or more independently selected halogen atoms, e.g., —O—CH₂F, —O—CHF₂, —O—CH₂C₁, —O—CCl₃, —O—CHClF, —O—CCl₂Br, etc.

When a range of values is given (e.g., C₁₋₆ alkyl), each value within the range as well as all intervening ranges are included. For example, “C₁₋₆ alkyl” includes C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

“Cycloalkyl” indicates a non-aromatic, fully saturated carbocyclic ring having the indicated number of carbon atoms, for example, 3 to 10, or 3 to 8, or 3 to 6, or 4 to 8 ring carbon atoms. Cycloalkyl groups may be monocyclic or polycyclic (e.g., bicyclic, tricyclic). Examples of cycloalkyl groups include cyclopropyl, cyclobutyl, cyclopentyl, and cyclohexyl, as well as bridged, caged, and spirocyclic ring groups (e.g., norbornane, bicyclo[2.2.2]octane, spiro[3.3]heptane). In addition, one ring of a polycyclic cycloalkyl group may be aromatic, provided the polycyclic cycloalkyl group is bound to the parent structure via a non-aromatic carbon. For example, a 1,2,3,4-tetrahydronaphthalen-1-yl group (wherein the moiety is bound to the parent structure via a non-aromatic carbon atom) is a cycloalkyl group, while 1,2,3,4-tetrahydronaphthalen-5-yl (wherein the moiety is bound to the parent structure via an aromatic carbon atom) is not considered a cycloalkyl group. Examples of polycyclic cycloalkyl groups consisting of a cycloalkyl group fused to an aromatic ring are described below.

“Heterocyclyl” indicates a non-aromatic, fully saturated ring having the indicated number of atoms (e.g., 3 to 10, or 3 to 7, or 4 to 8 membered heterocycloalkyl) made up of one or more heteroatoms (e.g., 1, 2, 3 or 4 heteroatoms) selected from N, O and S and with the remaining ring atoms being carbon. Heterocycloalkyl groups may be monocyclic or polycyclic (e.g., bicyclic, tricyclic). Examples of heterocycloalkyl groups include oxiranyl, aziridinyl, azetidinyl, pyrrolidinyl, imidazolidinyl, pyrazolidinyl, piperidinyl, piperazinyl, morpholinyl and thiomorpholinyl. Examples include thiomorpholine S-oxide and thiomorpholine S,S-dioxide. Examples of spirocyclic heterocycloalkyl groups include azaspiro[3.3]heptane, diazaspiro[3.3]heptane, diazaspiro[3.4]octane, and diazaspiro[3.5]nonane. In addition, one ring of a polycyclic heterocycloalkyl group may be aromatic (e.g., aryl or heteroaryl), provided the polycyclic heterocycloalkyl group is bound to the parent structure via a non-aromatic carbon or nitrogen atom. For example, a 1,2,3,4-tetrahydroquinolin-1-yl group (wherein the moiety is bound to the parent structure via a non-aromatic nitrogen atom) is considered a heterocycloalkyl group, while 1,2,3,4-tetrahydroquinolin-8-yl group (wherein the moiety is bound to the parent structure via an aromatic carbon atom) is not considered a heterocycloalkyl group.

“Halogen” or “halo” refers to fluorine, chlorine, bromine, or iodine.

“Phenyl” refers to

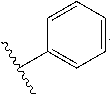 [SMILES: *c1ccccc1]

The phenyl moiety may be optionally substituted.

“Pyridinyl” refers to

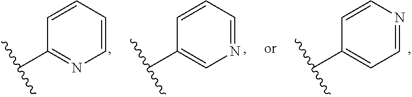

The pyridinyl moiety may be optionally substituted.

Unless otherwise indicated, compounds disclosed and/or described herein include all possible enantiomers, diastereomers, meso isomers and other stereoisomeric forms, including racemic mixtures, optically pure forms and intermediate mixtures thereof. Enantiomers, diastereomers, meso isomers and other stereoisomeric forms can be prepared using chiral synthons or chiral reagents, or resolved using conventional techniques. Unless specified otherwise, when the compounds disclosed and/or described herein contain olefinic double bonds or other centers of geometric asymmetry, it is intended that the compounds include both E and Z isomers. When the compounds described herein contain moieties capable of tautomerization, and unless specified otherwise, it is intended that the compounds include all possible tautomers.

“Protecting group” has the meaning conventionally associated with it in organic synthesis, i.e., a group that selectively blocks one or more reactive sites in a multifunctional compound such that a chemical reaction can be carried out selectively on another unprotected reactive site, and such that the group can readily be removed after the selective reaction is complete. A variety of protecting groups are disclosed, for example, in T. H. Greene and P. G. M. Wuts, Protective Groups in Organic Synthesis, Third Edition, John Wiley & Sons, New York (1999). For example, a “hydroxy protected form” contains at least one hydroxy group protected with a hydroxy protecting group. Likewise, amines and other reactive groups may similarly be protected.

The term “pharmaceutically acceptable salt” refers to a salt of any of the compounds herein which are known to be non-toxic and are commonly used in the pharmaceutical literature. In some embodiments, the pharmaceutically acceptable salt of a compound retains the biological effectiveness of the compounds described herein and are not biologically or otherwise undesirable. Examples of pharmaceutically acceptable salts can be found in Berge et al., Pharmaceutical Salts, J. Pharmaceutical Sciences, January 1977, 66(1), 1-19. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, lactic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, 2-hydroxyethylsulfonic acid, p-toluenesulfonic acid, stearic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines; substituted amines including naturally occurring substituted amines; cyclic amines; and basic ion exchange resins. Examples of organic bases include isopropylamine, trimethylamine, diethylamine, triethylamine, tripropylamine, and ethanolamine. In some embodiments, the pharmaceutically acceptable base addition salt is selected from ammonium, potassium, sodium, calcium, and magnesium salts.

If the compound described herein is obtained as an acid addition salt, the free base can be obtained by basifying a solution of the acid salt. Conversely, if the compound is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds (see, e.g., Berge et al., Pharmaceutical Salts, J. Pharmaceutical Sciences, January 1977, 66(1), 1-19). Those skilled in the art will recognize various synthetic methodologies that may be used to prepare pharmaceutically acceptable addition salts.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

The term “substituted” means that the specified group or moiety bears one or more substituents including, but not limited to, substituents such as alkoxy, acyl, acyloxy, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, cycloalkyl, cycloalkenyl, alkyl, alkenyl, alkynyl, heterocycloalkyl, heterocycloalkenyl, aralkyl, aminosulfonyl, sulfonylamino, sulfonyl, oxo, carbonylalkylenealkoxy and the like. The term “unsubstituted” means that the specified group bears no substituents. Where the term “substituted” is used to describe a structural system, the substitution is meant to occur at any valency-allowed position on the system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

The compounds disclosed and/or described herein can be enriched isotopic forms, e.g., enriched in the content of ²H, ³H, ¹¹C, ¹³C and/or ¹⁴C. In one embodiment, the compound contains at least one deuterium atom. Such deuterated forms can be made, for example, by the procedure described in U.S. Pat. Nos. 5,846,514 and 6,334,997. Such deuterated compounds may improve the efficacy and increase the duration of action of compounds disclosed and/or described herein. Deuterium substituted compounds can be synthesized using various methods, such as those described in: Dean, D., Recent Advances in the Synthesis and Applications of Radiolabeled Compounds for Drug Discovery and Development, Curr. Pharm. Des., 2000; 6 (10); Kabalka, G. et al., The Synthesis of Radiolabeled Compounds via Organometallic Intermediates, Tetrahedron, 1989, 45(21), 6601-21; and Evans, E., Synthesis of radiolabeled compounds, J. Radioanal. Chem., 1981, 64 (1-2), 9-32.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in pharmaceutical compositions is contemplated. Supplementary active ingredients can also be incorporated into the pharmaceutical compositions.

The terms “patient,” “individual,” and “subject” refer to an animal, such as a mammal, bird, or fish. In some embodiments, the patient or subject is a mammal. Mammals include, for example, mice, rats, dogs, cats, pigs, sheep, horses, cows and humans. In some embodiments, the patient or subject is a human, for example a human that has been or will be the object of treatment, observation or experiment. The compounds, compositions and methods described herein can be useful in both human therapy and veterinary applications.

As used herein, the term “therapeutic” refers to the ability to modulate CD38. As used herein, “modulation” refers to a change in activity as a direct or indirect response to the presence of a chemical entity as described herein, relative to the activity of in the absence of the chemical entity. The change may be an increase in activity or a decrease in activity, and may be due to the direct interaction of the chemical entity with the a target or due to the interaction of the chemical entity with one or more other factors that in turn affect the target's activity. For example, the presence of the chemical entity may, for example, increase or decrease the target activity by directly binding to the target, by causing (directly or indirectly) another factor to increase or decrease the target activity, or by (directly or indirectly) increasing or decreasing the amount of target present in the cell or organism. In some embodiments, the modulation is inhibition of CD38.

The term “therapeutically effective amount” or “effective amount” refers to that amount of a compound disclosed and/or described herein that is sufficient to affect treatment, as defined herein, when administered to a patient in need of such treatment. A therapeutically effective amount of a compound may be an amount sufficient to treat a disease responsive to modulation of CD38. The therapeutically effective amount will vary depending upon, for example, the subject and disease condition being treated, the weight and age of the subject, the severity of the disease condition, the particular compound, the dosing regimen to be followed, timing of administration, the manner of administration, all of which can readily be determined by one of ordinary skill in the art. The therapeutically effective amount may be ascertained experimentally, for example, by assaying blood concentration of the chemical entity, or theoretically, by calculating bioavailability.

“Treatment” (and related terms, such as “treat”, “treated”, “treating”) includes one or more of inhibiting a disease or disorder; slowing or arresting the development of clinical symptoms of a disease or disorder; and/or relieving a disease or disorder (i.e., causing relief from or regression of clinical symptoms). The term encompasses situations where the disease or disorder is already being experienced by a patient The term covers both complete and partial reduction of the condition or disorder, and complete or partial reduction of clinical symptoms of a disease or disorder. Thus, compounds described and/or disclosed herein may prevent an existing disease or disorder from worsening, assist in the management of the disease or disorder, and/or reduce or eliminate the disease or disorder.

“Prevention” (and related terms, such as “prevent”, “prevented”, “preventing”) of a disease or disorder includes causing the clinical symptoms of the disease or disorder not to develop. As such, the term encompasses situations where the disease or disorder is not currently being experienced but is expected to arise. When used in a preventative or prophylactic manner, the compounds disclosed and/or described herein may prevent a disease or disorder from developing or lessen the extent of a disease or disorder that may develop.

### Compounds

Compounds and salts thereof (such as pharmaceutically acceptable salts) are detailed herein, including in the Brief Summary and in the appended claims. Also provided are the use of all of the compounds described herein, including any and all stereoisomers, including geometric isomers (cis/trans), E/Z isomers, enantiomers, diastereomers, and mixtures thereof in any ratio including racemic mixtures, salts and solvates of the compounds described herein, as well as methods of making such compounds. Any compound described herein may also be referred to as a drug.

In one aspect, provided herein is a compound of formula (I):

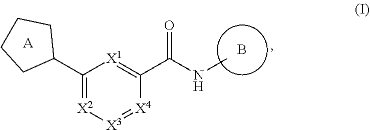

or a stereoisomer or tautomer thereof, or a pharmaceutically acceptable salt of any of the foregoing, wherein:

### X¹ is N or CH;

X² is N or C(R^{x}), wherein R^{x} is H, halo, or C₁₋₆alkyl;
X³ is N or C(R^{y}), wherein R^{y} is H, —OH, C₁₋₆alkoxy, C₃₋₁₀cycloalkyl, 3-10 membered heterocyclyl, or C₁₋₆alkyl,
wherein the C₁₋₆alkoxy of R^{y} is optionally substituted with one or more C₁₋₆alkoxy, the C₃₋₁₀cycloalkyl of R^{y} is optionally substituted with one or more halo, C₁₋₆alkoxy, or —OH, the 3-10 membered heterocyclyl of R^{y} is optionally substituted with one or more C₁₋₆alkyl, and the C₁₋₆alkyl of R^{y} is optionally substituted with one or more halo or —OH;
   X⁴ is N or C(R^{z}), wherein R^{z} is H, halo, —NH₂, C₁₋₆alkoxy, or C₁₋₆alkyl;
   provided that at most two of X¹, X², X³, and X⁴ are N;

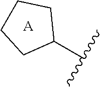

is:
(i)

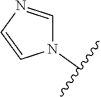 [SMILES: *n1ccnc1]

that is optionally substituted with one or more —C(O)—NH₂, or
(ii)

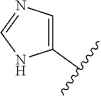 [SMILES: *c1cnc[nH]1]

that is optionally substituted with one or more C₁₋₆ alkyl, or
(iii)

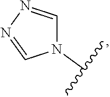 [SMILES: *n1cnnc1]

or
(iv)

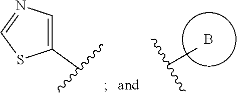

is:
(i) C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆haloalkyl), phenyl, phenoxy, or pyridinyl,
wherein the C₁₋₆alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
   (ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆alkyl, —C(O)—C₁₋₆ alkoxy, or phenyl, wherein the phenyl of R^{b} is optionally substituted with one or more C₁₋₆haloalkyl, or
   (iii) phenyl, wherein the phenyl is optionally substituted with one or more halo, or with C₁₋₆alkyl optionally substituted with —OH, or
   (iv) pyridinyl, wherein the pyridinyl is optionally substituted with one or more halo, C₁₋₆haloalkyl, C₁₋₆alkoxy optionally substituted with one or more halo, C₁₋₆alkyl optionally substituted with —OH, or —O—C₃₋₁₀cycloalkyl optionally substituted with one or more halo.

In some embodiments, the compound of formula (I) or the stereoisomer or tautomer thereof, or the pharmaceutically acceptable salt of any of the foregoing, is not a compound selected from the compounds of Table 1X, Table 2X, Table 3X, Table 4X, Table 5X, or Table 6X, or a stereoisomer or tautomer thereof, or a pharmaceutically acceptable salt of any of the foregoing.

TABLE 1X

| Compound | | |
|---|---|---|
| Number | Structure | Name |
| X-1 | 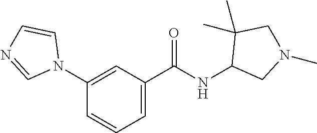 [SMILES: CN1CC(NC(=O)c2cccc(-n3ccnc3)c2)C(C)(C)C1] | 3-(1H-imidazol-1-yl)-N-(1,4,4- trimethylpyrrolidin-3-yl)benzamide |
| X-2 | 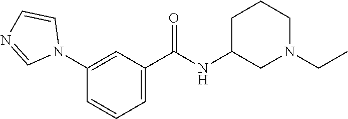 [SMILES: CCN1CCCC(NC(=O)c2cccc(-n3ccnc3)c2)C1] | N-(1-ethylpiperidin-3-yl)-3-(1H- imidazol-1-yl)benzamide |
| X-3 | 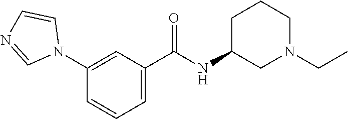 [SMILES: CCN1CCC[C@H](NC(=O)c2cccc(-n3ccnc3)c2)C1] | (S)-N-(1-ethylpiperidin-3-yl)-3- (1H-imidazol-1-yl)benzamide |
| X-4 | 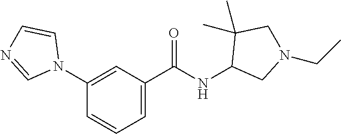 [SMILES: CCN1CC(NC(=O)c2cccc(-n3ccnc3)c2)C(C)(C)C1] | N-(1-ethyl-4,4-dimethylpyrrolidin- 3-yl)-3-(1H-imidazol-1- yl)benzamide |
| X-5 | 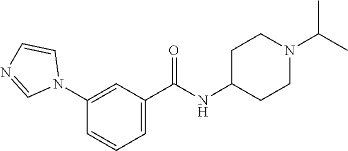 [SMILES: CC(C)N1CCC(NC(=O)c2cccc(-n3ccnc3)c2)CC1] | 3-(1H-imidazol-1-yl)-N-(1- isopropylpiperidin-4-yl)benzamide |
| X-6 | 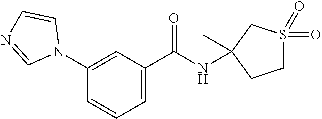 [SMILES: CC1(NC(=O)c2cccc(-n3ccnc3)c2)CCS(=O)(=O)C1] | 3-(1H-imidazol-1-yl)-N-(3-methyl- 1,1-dioxidotetrahydrothiophen-3- yl)benzamide |
| X-7 | 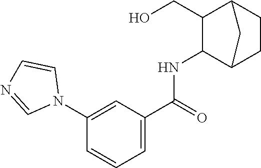 [SMILES: O=C(NC1C2CCC(C2)C1CO)c1cccc(-n2ccnc2)c1] | N-(3- (hydroxymethyl)bicyclo[2.2.1] heptan-2-yl)-3-(1H-imidazol-1- yl)benzamide |
| X-8 | 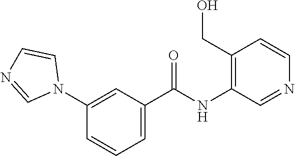 [SMILES: O=C(Nc1cnccc1CO)c1cccc(-n2ccnc2)c1] | N-(4-(hydroxymethyl)pyridin-3- yl)-3-(1H-imidazol-1-yl)benzamide |
| X-9 | 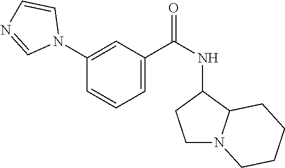 [SMILES: O=C(NC1CCN2CCCCC12)c1cccc(-n2ccnc2)c1] | 3-(1H-imidazol-1-yl)-N- (octahydroindolizin-1- yl)benzamide |
| X-10 | 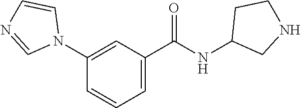 [SMILES: O=C(NC1CCNC1)c1cccc(-n2ccnc2)c1] | 3-(1H-imidazol-1-yl)-N- (pyrrolidin-3-yl)benzamide |
| X-11 | 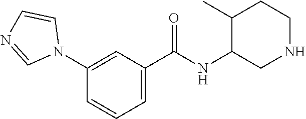 [SMILES: CC1CCNCC1NC(=O)c1cccc(-n2ccnc2)c1] | 3-(1H-imidazol-1-yl)-N-(4- methylpiperidin-3-yl)benzamide |
| X-12 | 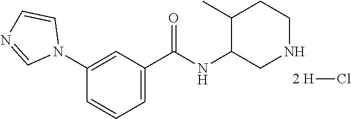 | 3-(1H-imidazol-1-yl)-N-(4- methylpiperidin-3-yl)benzamide dihydrochloride |
| X-13 | 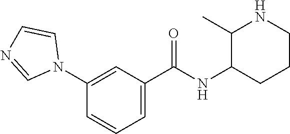 [SMILES: CC1NCCCC1NC(=O)c1cccc(-n2ccnc2)c1] | 3-(1H-imidazol-1-yl)-N-(2- methylpiperidin-3-yl)benzamide |
| X-14 | 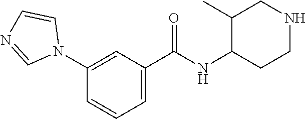 [SMILES: CC1CNCCC1NC(=O)c1cccc(-n2ccnc2)c1] | 3-(1H-imidazol-1-yl)-N-(3- methylpiperidin-4-yl)benzamide |
| X-15 | 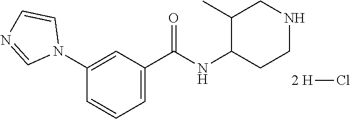 | 3-(1H-imidazol-1-yl)-N-(3- methylpiperidin-4-yl)benzamide dihydrochloride |
| X-16 | 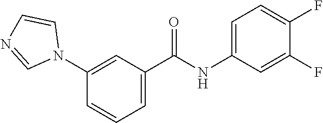 [SMILES: O=C(Nc1ccc(F)c(F)c1)c1cccc(-n2ccnc2)c1] | N-(3,4-difluorophenyl)-3-(1H- imidazol-1-yl)benzamide |
| X-17 | 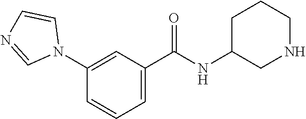 [SMILES: O=C(NC1CCCNC1)c1cccc(-n2ccnc2)c1] | 3-(1H-imidazol-1-yl)-N-(piperidin- 3-yl)benzamide |
| X-18 | 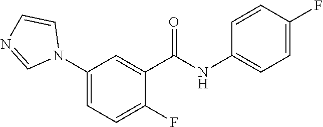 [SMILES: O=C(Nc1ccc(F)cc1)c1cc(-n2ccnc2)ccc1F] | 2-fluoro-N-(4-fluorophenyl)-5-(1H- imidazol-1-yl)benzamide |
| X-19 | 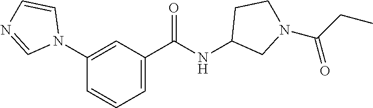 [SMILES: CCC(=O)N1CCC(NC(=O)c2cccc(-n3ccnc3)c2)C1] | 3-(1H-imidazol-1-yl)-N-(1- propionylpyrrolidin-3- yl)benzamide |
| X-20 | 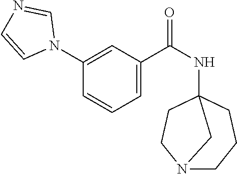 [SMILES: O=C(NC12CCCN(CC1)C2)c1cccc(-n2ccnc2)c1] | N-(1-azabicyclo[3.2.1]octan-5-yl)- 3-(1H-imidazol-1-yl)benzamide |
| X-21 | 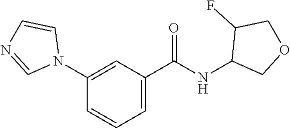 [SMILES: O=C(NC1COCC1F)c1cccc(-n2ccnc2)c1] | N-(4-fluorotetrahydrofuran-3-yl)-3- (1H-imidazol-1-yl)benzamide |
| X-22 | 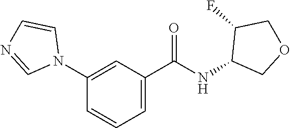 [SMILES: O=C(N[C@H]1COC[C@H]1F)c1cccc(-n2ccnc2)c1] | N-((3S,4S)-4- fluorotetrahydrofuran-3-yl)-3-(1H- imidazol-1-yl)benzamide |
| X-23 | 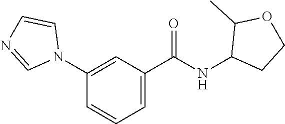 [SMILES: CC1OCCC1NC(=O)c1cccc(-n2ccnc2)c1] | 3-(1H-imidazol-1-yl)-N-(2- methyltetrahydrofuran-3- yl)benzamide |
| X-24 | 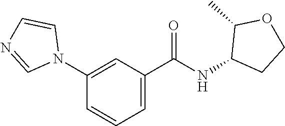 [SMILES: C[C@@H]1OCC[C@@H]1NC(=O)c1cccc(-n2ccnc2)c1] | 3-(1H-imidazol-1-yl)-N-((2S,3S)-2- methyltetrahydrofuran-3- yl)benzamide |
| X-25 | 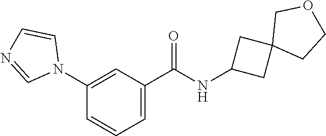 [SMILES: O=C(NC1CC2(CCOC2)C1)c1cccc(-n2ccnc2)c1] | 3-(1H-imidazol-1-yl)-N-(6- oxaspiro[3.4]octan-2-yl)benzamide |
| X-26 | 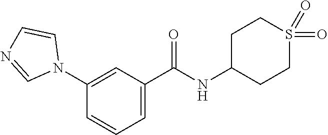 [SMILES: O=C(NC1CCS(=O)(=O)CC1)c1cccc(-n2ccnc2)c1] | N-(1,1-dioxidotetrahydro-2H- thiopyran-4-yl)-3-(1H-imidazol-1- yl)benzamide |
| X-27 | 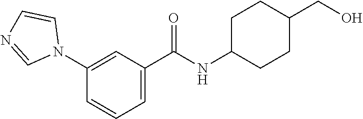 [SMILES: O=C(NC1CCC(CO)CC1)c1cccc(-n2ccnc2)c1] | N-(4-(hydroxymethyl)cyclohexyl)- 3-(1H-imidazol-1-yl)benzamide |
| X-28 | 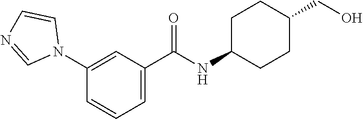 [SMILES: O=C(N[C@H]1CC[C@H](CO)CC1)c1cccc(-n2ccnc2)c1] | N-((1r,4r)-4- (hydroxymethyl)cyclohexyl)-3- (1H-imidazol-1-yl)benzamide |
| X-29 | 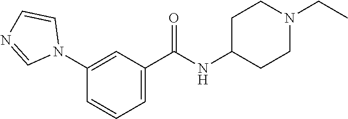 [SMILES: CCN1CCC(NC(=O)c2cccc(-n3ccnc3)c2)CC1] | N-(1-ethylpiperidin-4-yl)-3-(1H- imidazol-1-yl)benzamide |
| X-30 | 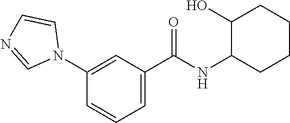 [SMILES: O=C(NC1CCCCC1O)c1cccc(-n2ccnc2)c1] | N-(2-hydroxycyclohexyl)-3-(1H- imidazol-1-yl)benzamide |
| X-31 | 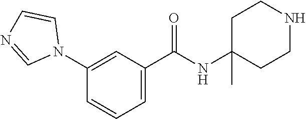 [SMILES: CC1(NC(=O)c2cccc(-n3ccnc3)c2)CCNCC1] | 3-(1H-imidazol-1-yl)-N-(4- methylpiperidin-4-yl)benzamide |
| X-32 | 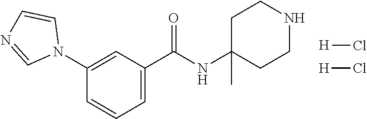 | 3-(1H-imidazol-1-yl)-N-(4- methylpiperidin-4-yl)benzamide dihydrochloride |
| X-33 | 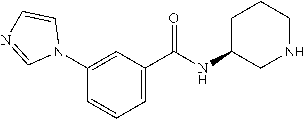 [SMILES: O=C(N[C@H]1CCCNC1)c1cccc(-n2ccnc2)c1] | (S)-3-(1H-imidazol-1-yl)-N- (piperidin-3-yl)benzamide |
| X-34 | 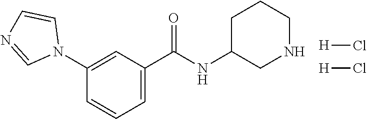 | 3-(1H-imidazol-1-yl)-N-(piperidin- 3-yl)benzamide dihydrochloride |
| X-35 | 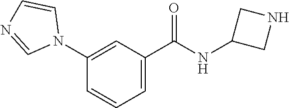 [SMILES: O=C(NC1CNC1)c1cccc(-n2ccnc2)c1] | N-(azetidin-3-yl)-3-(1H-imidazol- 1-yl)benzamide |
| X-36 | 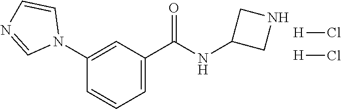 | N-(azetidin-3-yl)-3-(1H-imidazol- 1-yl)benzamide dihydrochloride |
| X-37 | 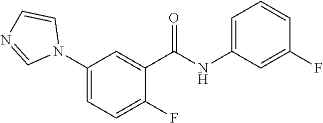 [SMILES: O=C(Nc1cccc(F)c1)c1cc(-n2ccnc2)ccc1F] | 2-fluoro-N-(3-fluorophenyl)-5- (1H-imidazol-1-yl)benzamide |
| X-38 | 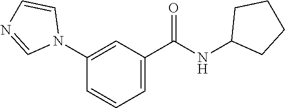 [SMILES: O=C(NC1CCCC1)c1cccc(-n2ccnc2)c1] | N-cyclopentyl-3-(1H-imidazol-1- yl)benzamide |
| X-39 | 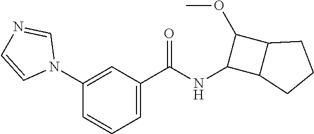 [SMILES: COC1C2CCCC2C1NC(=O)c1cccc(-n2ccnc2)c1] | 3-(1H-imidazol-1-yl)-N-(7- methoxybicyclo[3.2.0]heptan-6- yl)benzamide |
| X-40 | 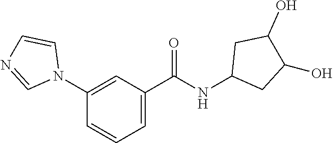 [SMILES: O=C(NC1CC(O)C(O)C1)c1cccc(-n2ccnc2)c1] | N-(3,4-dihydroxycyclopentyl)-3- (1H-imidazol-1-yl)benzamide |
| X-41 | 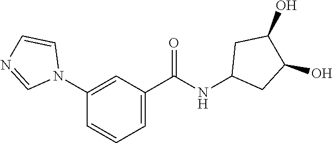 [SMILES: O=C(NC1C[C@@H](O)[C@@H](O)C1)c1cccc(-n2ccnc2)c1] | N-((3R,4S)-3,4- dihydroxycyclopentyl)-3-(1H- imidazol-1-yl)benzamide |
| X-42 | 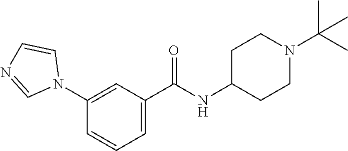 [SMILES: CC(C)(C)N1CCC(NC(=O)c2cccc(-n3ccnc3)c2)CC1] | N-(1-(tert-butyl)piperidin-4-yl)-3- (1H-imidazol-1-yl)benzamide |
| X-43 | 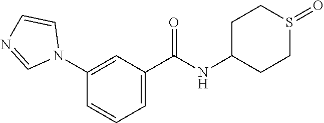 [SMILES: O=C(NC1CCS(=O)CC1)c1cccc(-n2ccnc2)c1] | 3-(1H-imidazol-1-yl)-N-(1- oxidotetrahydro-2H-thiopyran-4- yl)benzamide |
| X-44 | 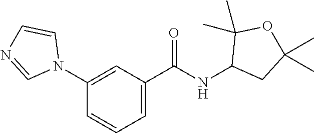 [SMILES: CC1(C)CC(NC(=O)c2cccc(-n3ccnc3)c2)C(C)(C)O1] | 3-(1H-imidazol-1-yl)-N-(2,2,5,5- tetramethyltetrahydrofuran-3- yl)benzamide |
| X-45 | 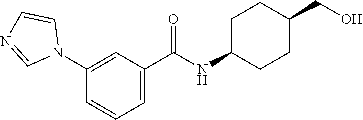 [SMILES: O=C(N[C@H]1CC[C@@H](CO)CC1)c1cccc(-n2ccnc2)c1] | N-((1s,4s)-4- (hydroxymethyl)cyclohexyl)-3- (1H-imidazol-1-yl)benzamide |
| X-46 | 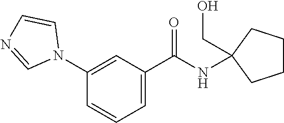 [SMILES: O=C(NC1(CO)CCCC1)c1cccc(-n2ccnc2)c1] | N-(1-(hydroxymethyl)cyclopentyl)- 3-(1H-imidazol-1-yl)benzamide |
| X-47 | 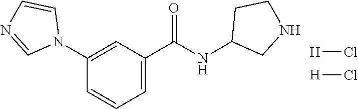 | 3-(1H-imidazol-1-yl)-N- (pyrrolidin-3-yl)benzamide dihydrochloride |
| X-48 | 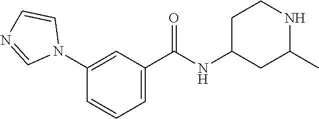 [SMILES: CC1CC(NC(=O)c2cccc(-n3ccnc3)c2)CCN1] | 3-(1H-imidazol-1-yl)-N-(2- methylpiperidin-4-yl)benzamide |
| X-49 | 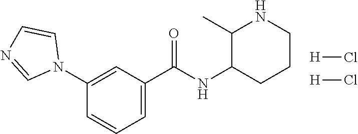 | 3-(1H-imidazol-1-yl)-N-(2- methylpiperidin-3-yl)benzamide dihydrochloride |
| X-50 | 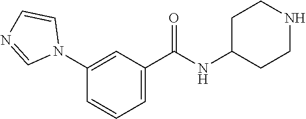 [SMILES: O=C(NC1CCNCC1)c1cccc(-n2ccnc2)c1] | 3-(1H-imidazol-1-yl)-N-(piperidin- 4-yl)benzamide |
| X-51 | 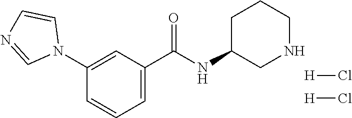 | (S)-3-(1H-imidazol-1-yl)-N- (piperidin-3-yl)benzamide dihydrochloride |
| X-52 | 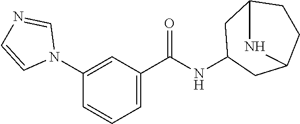 [SMILES: O=C(NC1CC2CCC(C1)N2)c1cccc(-n2ccnc2)c1] | N-(8-azabicyclo[3.2.1]octan-3-yl)- 3-(1H-imidazol-1-yl)benzamide |
| X-53 | 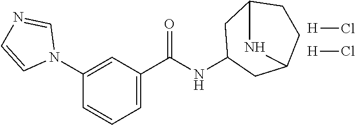 | N-(8-azabicyclo[3.2.1]octan-3-yl)- 3-(1H-imidazol-1-yl)benzamide dihydrochloride |
| X-54 | 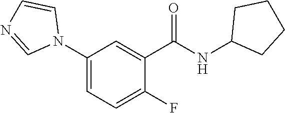 [SMILES: O=C(NC1CCCC1)c1cc(-n2ccnc2)ccc1F] | N-cyclopentyl-2-fluoro-5-(1H- imidazol-1-yl)benzamide |
| X-55 | 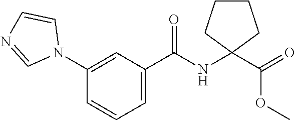 [SMILES: COC(=O)C1(NC(=O)c2cccc(-n3ccnc3)c2)CCCC1] | methyl 1-(3-(1H-imidazol-1- yl)benzamido)cyclopentane-1- carboxylate |
| X-56 | 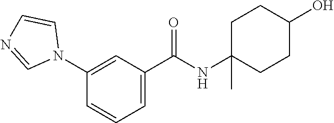 [SMILES: CC1(NC(=O)c2cccc(-n3ccnc3)c2)CCC(O)CC1] | N-(4-hydroxy-1- methylcyclohexyl)-3-(1H-imidazol- 1-yl)benzamide |
| X-57 | 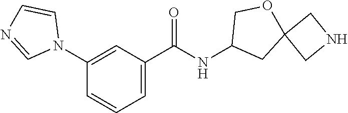 [SMILES: O=C(NC1COC2(CNC2)C1)c1cccc(-n2ccnc2)c1] | 3-(1H-imidazol-1-yl)-N-(5-oxa-2- azaspiro[3.4]octan-7-yl)benzamide |
| X-58 | 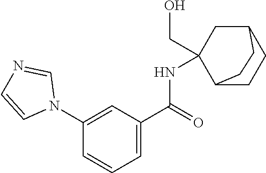 [SMILES: O=C(NC1(CO)CC2CCC1CC2)c1cccc(-n2ccnc2)c1] | N-(2- (hydroxymethyl)bicyclo[2.2.2] octan-2-yl)-3-(1H-imidazol-1- yl)benzamide |
| X-59 | 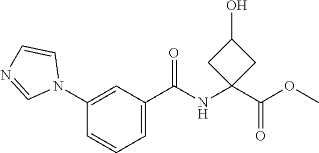 [SMILES: COC(=O)C1(NC(=O)c2cccc(-n3ccnc3)c2)CC(O)C1] | methyl 1-(3-(1H-imidazol-1- yl)benzamido)-3- hydroxycyclobutane-1-carboxylate |
| X-60 | 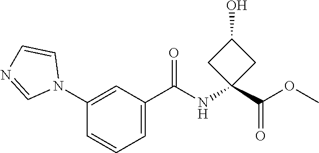 [SMILES: COC(=O)[C@]1(NC(=O)c2cccc(-n3ccnc3)c2)C[C@@H](O)C1] | methyl (1s,3s)-1-(3-(1H-imidazol- 1-yl)benzamido)-3- hydroxycyclobutane-1-carboxylate |
| X-61 | 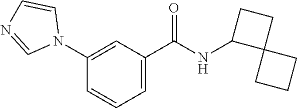 [SMILES: O=C(NC1CCC12CCC2)c1cccc(-n2ccnc2)c1] | 3-(1H-imidazol-1-yl)-N- (spiro[3.3]heptan-1-yl)benzamide |
| X-62 | 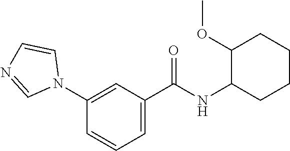 [SMILES: COC1CCCCC1NC(=O)c1cccc(-n2ccnc2)c1] | 3-(1H-imidazol-1-yl)-N-(2- methoxycyclohexyl)benzamide |
| X-63 | 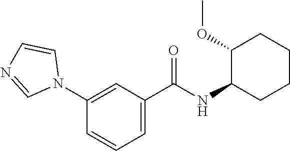 [SMILES: CO[C@@H]1CCCC[C@H]1NC(=O)c1cccc(-n2ccnc2)c1] | 3-(1H-imidazol-1-yl)-N-((1R,2R)- 2-methoxycyclohexyl)benzamide |
| X-64 | 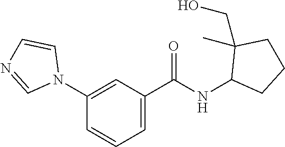 [SMILES: CC1(CO)CCCC1NC(=O)c1cccc(-n2ccnc2)c1] | N-(2-(hydroxymethyl)-2- methylcyclopentyl)-3-(1H- imidazol-1-yl)benzamide |
| X-65 | 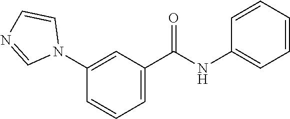 [SMILES: O=C(Nc1ccccc1)c1cccc(-n2ccnc2)c1] | 3-(1H-imidazol-1-yl)-N- phenylbenzamide |
| X-66 | 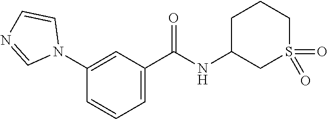 [SMILES: O=C(NC1CCCS(=O)(=O)C1)c1cccc(-n2ccnc2)c1] | N-(1,1-dioxidotetrahydro-2H- thiopyran-3-yl)-3-(1H-imidazol-1- yl)benzamide |
| X-67 | 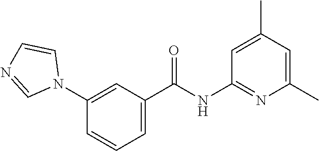 [SMILES: Cc1cc(C)nc(NC(=O)c2cccc(-n3ccnc3)c2)c1] | N-(4,6-dimethylpyridin-2-yl)-3- (1H-imidazol-1-yl)benzamide |
| X-68 | 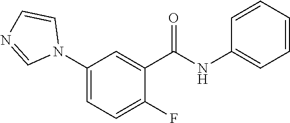 [SMILES: O=C(Nc1ccccc1)c1cc(-n2ccnc2)ccc1F] | 2-fluoro-5-(1H-imidazol-1-yl)-N- phenylbenzamide |
| X-69 | 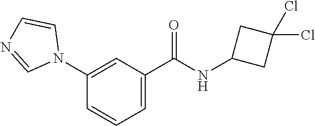 [SMILES: O=C(NC1CC(Cl)(Cl)C1)c1cccc(-n2ccnc2)c1] | N-(3,3-dichlorocyclobutyl)-3-(1H- imidazol-1-yl)benzamide |
| X-70 | 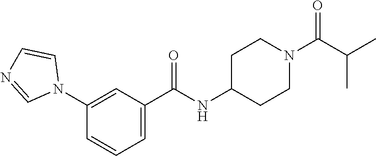 [SMILES: CC(C)C(=O)N1CCC(NC(=O)c2cccc(-n3ccnc3)c2)CC1] | 3-(1H-imidazol-1-yl)-N-(1- isobutyrylpiperidin-4-yl)benzamide |
| X-71 | 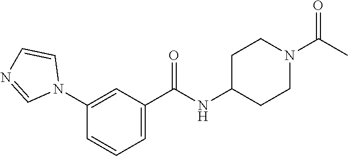 [SMILES: CC(=O)N1CCC(NC(=O)c2cccc(-n3ccnc3)c2)CC1] | N-(1-acetylpiperidin-4-yl)-3-(1H- imidazol-1-yl)benzamide |
| X-72 | 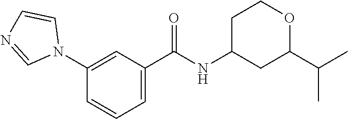 [SMILES: CC(C)C1CC(NC(=O)c2cccc(-n3ccnc3)c2)CCO1] | 3-(1H-imidazol-1-yl)-N-(2- isopropyltetrahydro-2H-pyran-4- yl)benzamide |
| X-73 | 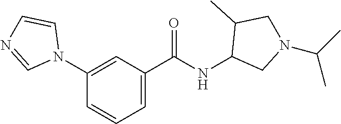 [SMILES: CC1CN(C(C)C)CC1NC(=O)c1cccc(-n2ccnc2)c1] | 3-(1H-imidazol-1-yl)-N-(1- isopropyl-4-methylpyrrolidin-3- yl)benzamide |
| X-74 | 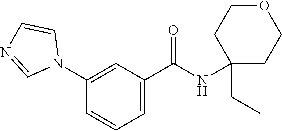 [SMILES: CCC1(NC(=O)c2cccc(-n3ccnc3)c2)CCOCC1] | N-(4-ethyltetrahydro-2H-pyran-4- yl)-3-(1H-imidazol-1-yl)benzamide |
| X-75 | 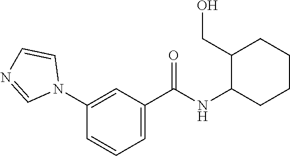 [SMILES: O=C(NC1CCCCC1CO)c1cccc(-n2ccnc2)c1] | N-(2-(hydroxymethyl)cyclohexyl)- 3-(1H-imidazol-1-yl)benzamide |
| X-76 | 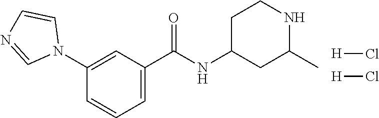 | 3-(1H-imidazol-1-yl)-N-(2- methylpiperidin-4-yl)benzamide dihydrochloride |
| X-77 | 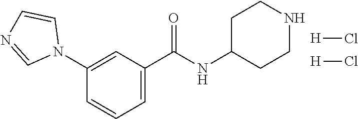 | 3-(1H-imidazol-1-yl)-N-(piperidin- 4-yl)benzamide dihydrochloride |
| X-78 | 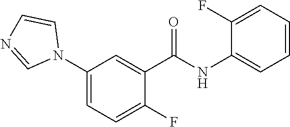 [SMILES: O=C(Nc1ccccc1F)c1cc(-n2ccnc2)ccc1F] | 2-fluoro-N-(2-fluorophenyl)-5-(1H- imidazol-1-yl)benzamide |
| X-79 | 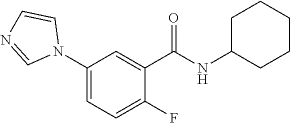 [SMILES: O=C(NC1CCCCC1)c1cc(-n2ccnc2)ccc1F] | N-cyclohexyl-2-fluoro-5-(1H- imidazol-1-yl)benzamide |
| X-80 | 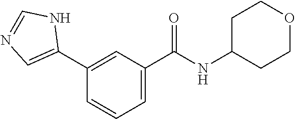 [SMILES: O=C(NC1CCOCC1)c1cccc(-c2cnc[nH]2)c1] | 3-(1H-imidazol-5-yl)-N- (tetrahydro-2H-pyran-4- yl)benzamide |
| X-81 | 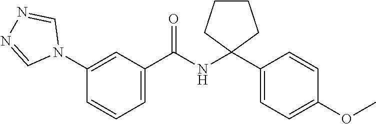 [SMILES: COc1ccc(C2(NC(=O)c3cccc(-n4cnnc4)c3)CCCC2)cc1] | N-(1-(4- methoxyphenyl)cyclopentyl)-3- (4H-1,2,4-triazol-4-yl)benzamide |
| X-82 | 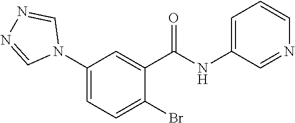 [SMILES: O=C(Nc1cccnc1)c1cc(-n2cnnc2)ccc1Br] | 2-bromo-N-(pyridin-3-yl)-5-(4H- 1,2,4-triazol-4-yl)benzamide |
| X-83 | 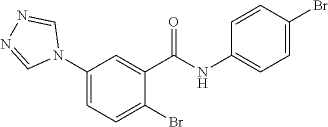 [SMILES: O=C(Nc1ccc(Br)cc1)c1cc(-n2cnnc2)ccc1Br] | 2-bromo-N-(4-bromophenyl)-5- (4H-1,2,4-triazol-4-yl)benzamide |
| X-84 | 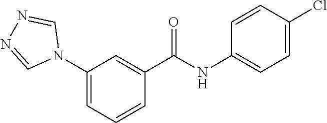 [SMILES: O=C(Nc1ccc(Cl)cc1)c1cccc(-n2cnnc2)c1] | N-(4-chlorophenyl)-3-(4H-1,2,4- triazol-4-yl)benzamide |
| X-85 | 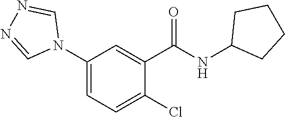 [SMILES: O=C(NC1CCCC1)c1cc(-n2cnnc2)ccc1Cl] | 2-chloro-N-cyclopentyl-5-(4H- 1,2,4-triazol-4-yl)benzamide |
| X-86 | 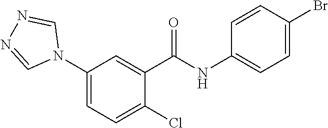 [SMILES: O=C(Nc1ccc(Br)cc1)c1cc(-n2cnnc2)ccc1Cl] | N-(4-bromophenyl)-2-chloro-5- (4H-1,2,4-triazol-4-yl)benzamide |
| X-87 | 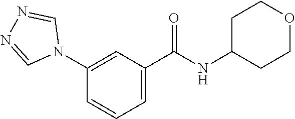 [SMILES: O=C(NC1CCOCC1)c1cccc(-n2cnnc2)c1] | N-(tetrahydro-2H-pyran-4-yl)-3- (4H-1,2,4-triazol-4-yl)benzamide |
| X-88 | 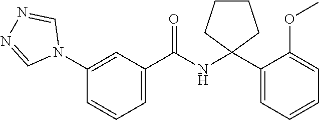 [SMILES: COc1ccccc1C1(NC(=O)c2cccc(-n3cnnc3)c2)CCCC1] | N-(1-(2- methoxyphenyl)cyclopentyl)-3- (4H-1,2,4-triazol-4-yl)benzamide |
| X-89 | 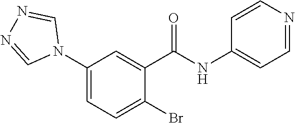 [SMILES: O=C(Nc1ccncc1)c1cc(-n2cnnc2)ccc1Br] | 2-bromo-N-(pyridin-4-yl)-5-(4H- 1,2,4-triazol-4-yl)benzamide |
| X-90 | 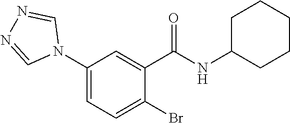 [SMILES: O=C(NC1CCCCC1)c1cc(-n2cnnc2)ccc1Br] | 2-bromo-N-cyclohexyl-5-(4H- 1,2,4-triazol-4-yl)benzamide |
| X-91 | 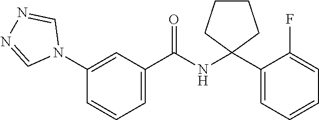 [SMILES: O=C(NC1(c2ccccc2F)CCCC1)c1cccc(-n2cnnc2)c1] | N-(1-(2-fluorophenyl)cyclopentyl)- 3-(4H-1,2,4-triazol-4-yl)benzamide |
| X-92 | 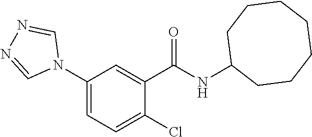 [SMILES: O=C(NC1CCCCCCC1)c1cc(-n2cnnc2)ccc1Cl] | 2-chloro-N-cyclooctyl-5-(4H-1,2,4- triazol-4-yl)benzamide |
| X-93 | 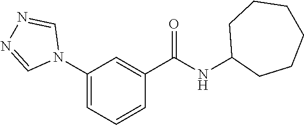 [SMILES: O=C(NC1CCCCCC1)c1cccc(-n2cnnc2)c1] | N-cycloheptyl-3-(4H-1,2,4-triazol- 4-yl)benzamide |
| X-94 | 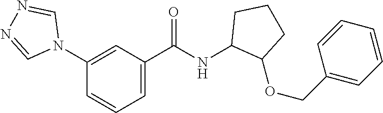 [SMILES: O=C(NC1CCCC1OCc1ccccc1)c1cccc(-n2cnnc2)c1] | N-(2-(benzyloxy)cyclopentyl)-3- (4H-1,2,4-triazol-4-yl)benzamide |
| X-95 | 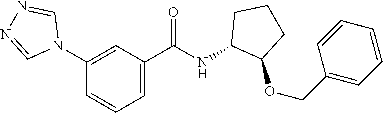 [SMILES: O=C(N[C@@H]1CCC[C@H]1OCc1ccccc1)c1cccc(-n2cnnc2)c1] | N-((1R,2R)-2- (benzyloxy)cyclopentyl)-3-(4H- 1,2,4-triazol-4-yl)benzamide |
| X-96 | 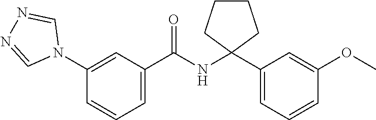 [SMILES: COc1cccc(C2(NC(=O)c3cccc(-n4cnnc4)c3)CCCC2)c1] | N-(1-(3- methoxyphenyl)cyclopentyl)-3- (4H-1,2,4-triazol-4-yl)benzamide |
| X-97 | 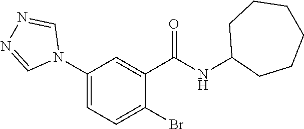 [SMILES: O=C(NC1CCCCCC1)c1cc(-n2cnnc2)ccc1Br] | 2-bromo-N-cycloheptyl-5-(4H- 1,2,4-triazol-4-yl)benzamide |
| X-98 | 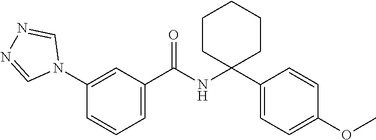 [SMILES: COc1ccc(C2(NC(=O)c3cccc(-n4cnnc4)c3)CCCCC2)cc1] | N-(1-(4- methoxyphenyl)cyclohexyl)-3-(4H- 1,2,4-triazol-4-yl)benzamide |
| X-99 | 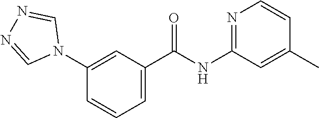 [SMILES: Cc1ccnc(NC(=O)c2cccc(-n3cnnc3)c2)c1] | N-(4-methylpyridin-2-yl)-3-(4H- 1,2,4-triazol-4-yl)benzamide |
| X-100 | 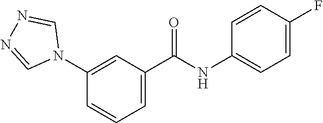 [SMILES: O=C(Nc1ccc(F)cc1)c1cccc(-n2cnnc2)c1] | N-(4-fluorophenyl)-3-(4H-1,2,4- triazol-4-yl)benzamide |
| X-101 | 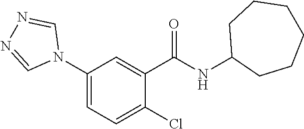 [SMILES: O=C(NC1CCCCCC1)c1cc(-n2cnnc2)ccc1Cl] | 2-chloro-N-cycloheptyl-5-(4H- 1,2,4-triazol-4-yl)benzamide |
| X-102 | 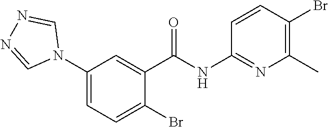 [SMILES: Cc1nc(NC(=O)c2cc(-n3cnnc3)ccc2Br)ccc1Br] | 2-bromo-N-(5-bromo-6- methylpyridin-2-yl)-5-(4H-1,2,4- triazol-4-yl)benzamide |
| X-103 | 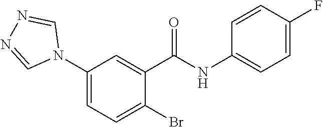 [SMILES: O=C(Nc1ccc(F)cc1)c1cc(-n2cnnc2)ccc1Br] | 2-bromo-N-(4-fluorophenyl)-5- (4H-1,2,4-triazol-4-yl)benzamide |
| X-104 | 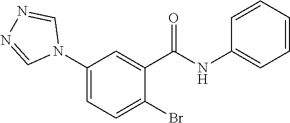 [SMILES: O=C(Nc1ccccc1)c1cc(-n2cnnc2)ccc1Br] | 2-bromo-N-phenyl-5-(4H-1,2,4- triazol-4-yl)benzamide |
| X-105 | 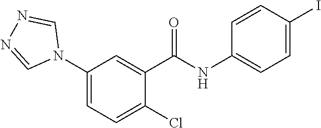 [SMILES: O=C(Nc1ccc(I)cc1)c1cc(-n2cnnc2)ccc1Cl] | 2-chloro-N-(4-iodophenyl)-5-(4H- 1,2,4-triazol-4-yl)benzamide |
| X-106 | 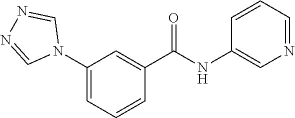 [SMILES: O=C(Nc1cccnc1)c1cccc(-n2cnnc2)c1] | N-(pyridin-3-yl)-3-(4H-1,2,4- triazol-4-yl)benzamide |
| X-107 | 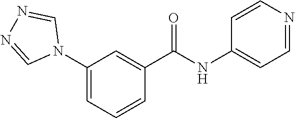 [SMILES: O=C(Nc1ccncc1)c1cccc(-n2cnnc2)c1] | N-(pyridin-4-yl)-3-(4H-1,2,4- triazol-4-yl)benzamide |
| X-108 | 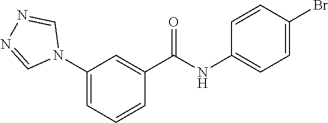 [SMILES: O=C(Nc1ccc(Br)cc1)c1cccc(-n2cnnc2)c1] | N-(4-bromophenyl)-3-(4H-1,2,4- triazol-4-yl)benzamide |
| X-109 | 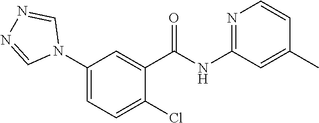 [SMILES: Cc1ccnc(NC(=O)c2cc(-n3cnnc3)ccc2Cl)c1] | 2-chloro-N-(4-methylpyridin-2-yl)- 5-(4H-1,2,4-triazol-4-yl)benzamide |
| X-110 | 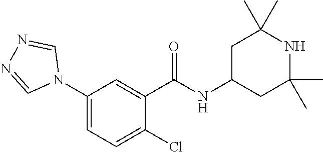 [SMILES: CC1(C)CC(NC(=O)c2cc(-n3cnnc3)ccc2Cl)CC(C)(C)N1] | 2-chloro-N-(2,2,6,6- tetramethylpiperidin-4-yl)-5-(4H- 1,2,4-triazol-4-yl)benzamide |
| X-111 | 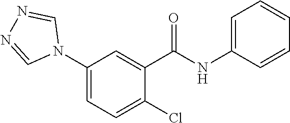 [SMILES: O=C(Nc1ccccc1)c1cc(-n2cnnc2)ccc1Cl] | 2-chloro-N-phenyl-5-(4H-1,2,4- triazol-4-yl)benzamide |
| X-112 | 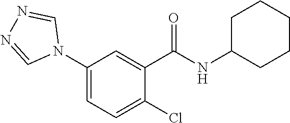 [SMILES: O=C(NC1CCCCC1)c1cc(-n2cnnc2)ccc1Cl] | 2-chloro-N-cyclohexyl-5-(4H- 1,2,4-triazol-4-yl)benzamide |
| X-113 | 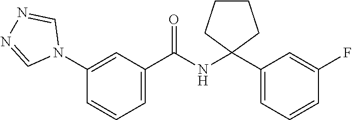 [SMILES: O=C(NC1(c2cccc(F)c2)CCCC1)c1cccc(-n2cnnc2)c1] | N-(1-(3-fluorophenyl)cyclopentyl)- 3-(4H-1,2,4-triazol-4-yl)benzamide |
| X-114 | 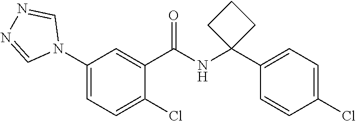 [SMILES: O=C(NC1(c2ccc(Cl)cc2)CCC1)c1cc(-n2cnnc2)ccc1Cl] | 2-chloro-N-(1-(4- chlorophenyl)cyclobutyl)-5-(4H- 1,2,4-triazol-4-yl)benzamide |
| X-115 | 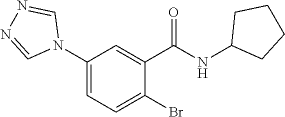 [SMILES: O=C(NC1CCCC1)c1cc(-n2cnnc2)ccc1Br] | 2-bromo-N-cyclopentyl-5-(4H- 1,2,4-triazol-4-yl)benzamide |
| X-116 | 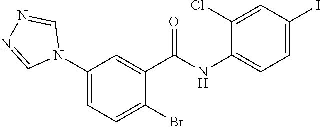 [SMILES: O=C(Nc1ccc(I)cc1Cl)c1cc(-n2cnnc2)ccc1Br] | 2-bromo-N-(2-chloro-4- iodophenyl)-5-(4H-1,2,4-triazol-4- yl)benzamide |
| X-117 | 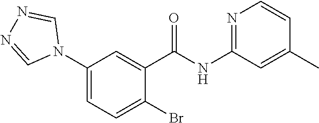 [SMILES: Cc1ccnc(NC(=O)c2cc(-n3cnnc3)ccc2Br)c1] | 2-bromo-N-(4-methylpyridin-2-yl)- 5-(4H-1,2,4-triazol-4-yl)benzamide |
| X-118 | 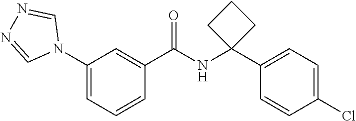 [SMILES: O=C(NC1(c2ccc(Cl)cc2)CCC1)c1cccc(-n2cnnc2)c1] | N-(1-(4-chlorophenyl)cyclobutyl)- 3-(4H-1,2,4-triazol-4-yl)benzamide |
| X-119 | 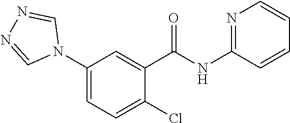 [SMILES: O=C(Nc1ccccn1)c1cc(-n2cnnc2)ccc1Cl] | 2-chloro-N-(pyridin-2-yl)-5-(4H- 1,2,4-triazol-4-yl)benzamide |
| X-120 | 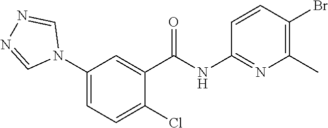 [SMILES: Cc1nc(NC(=O)c2cc(-n3cnnc3)ccc2Cl)ccc1Br] | N-(5-bromo-6-methylpyridin-2-yl)- 2-chloro-5-(4H-1,2,4-triazol-4- yl)benzamide |
| X-121 | 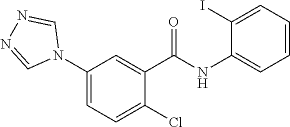 [SMILES: O=C(Nc1ccccc1I)c1cc(-n2cnnc2)ccc1Cl] | 2-chloro-N-(2-iodophenyl)-5-(4H- 1,2,4-triazol-4-yl)benzamide |
| X-122 | 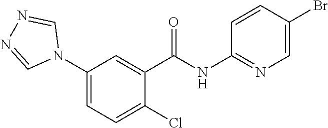 [SMILES: O=C(Nc1ccc(Br)cn1)c1cc(-n2cnnc2)ccc1Cl] | N-(5-bromopyridin-2-yl)-2-chloro- 5-(4H-1,2,4-triazol-4-yl)benzamide |
| X-123 | 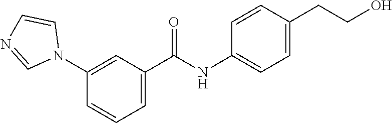 [SMILES: O=C(Nc1ccc(CCO)cc1)c1cccc(-n2ccnc2)c1] | N-(4-(2-hydroxyethyl)phenyl)-3- (1H-imidazol-1-yl)benzamide |
| X-124 | 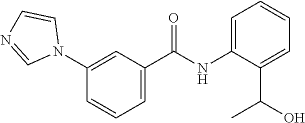 [SMILES: CC(O)c1ccccc1NC(=O)c1cccc(-n2ccnc2)c1] | N-(2-(1-hydroxyethyl)phenyl)-3- (1H-imidazol-1-yl)benzamide |

TABLE 2X

| Compound | | |
|---|---|---|
| Number | Structure | Name |
| X-125 | 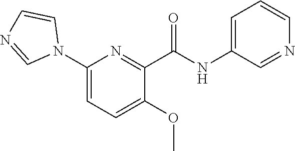 [SMILES: COc1ccc(-n2ccnc2)nc1C(=O)Nc1cccnc1] | 6-(1H-imidazol-1-yl)-3-methoxy- N-(pyridin-3-yl)picolinamide |
| X-126 | 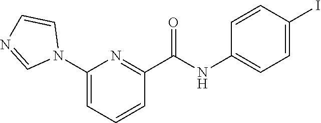 [SMILES: O=C(Nc1ccc(I)cc1)c1cccc(-n2ccnc2)n1] | 6-(1H-imidazol-1-yl)-N-(4- iodophenyl)picolinamide |
| X-127 | 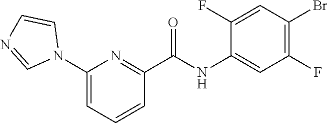 [SMILES: O=C(Nc1cc(F)c(Br)cc1F)c1cccc(-n2ccnc2)n1] | N-(4-bromo-2,5-difluorophenyl)-6- (1H-imidazol-1-yl)picolinamide |
| X-128 | 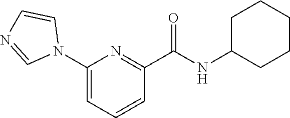 [SMILES: O=C(NC1CCCCC1)c1cccc(-n2ccnc2)n1] | N-cyclohexyl-6-(1H-imidazol-1- yl)picolinamide |
| X-129 | 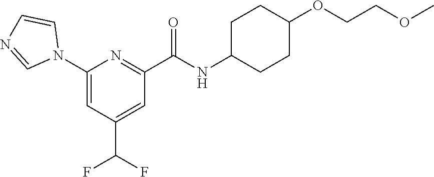 [SMILES: COCCOC1CCC(NC(=O)c2cc(C(F)F)cc(-n3ccnc3)n2)CC1] | 4-(difluoromethyl)-6-(1H-imidazol- 1-yl)-N-(4-(2- methoxyethoxy)cyclohexyl) picolinamide |
| X-130 | 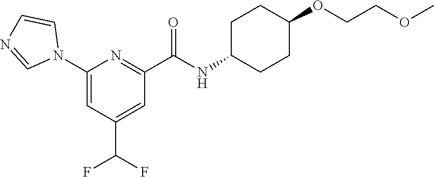 [SMILES: COCCO[C@H]1CC[C@H](NC(=O)c2cc(C(F)F)cc(-n3ccnc3)n2)CC1] | 4-(difluoromethyl)-6-(1H-imidazol- 1-yl)-N-((1r,4r)-4-(2- methoxyethoxy)cyclohexyl) picolinamide |
| X-131 | 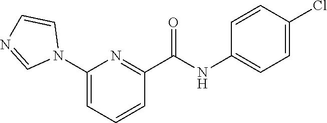 [SMILES: O=C(Nc1ccc(Cl)cc1)c1cccc(-n2ccnc2)n1] | N-(4-chlorophenyl)-6-(1H- imidazol-1-yl)picolinamide |
| X-132 | 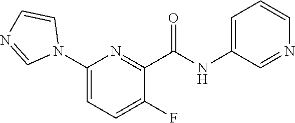 [SMILES: O=C(Nc1cccnc1)c1nc(-n2ccnc2)ccc1F] | 3-fluoro-6-(1H-imidazol-1-yl)-N- (pyridin-3-yl)picolinamide |
| X-133 | 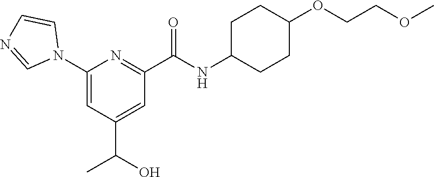 [SMILES: COCCOC1CCC(NC(=O)c2cc(C(C)O)cc(-n3ccnc3)n2)CC1] | 4-(1-hydroxyethyl)-6-(1H- imidazol-1-yl)-N-(4-(2- methoxyethoxy)cyclohexyl) picolinamide |
| X-134 | 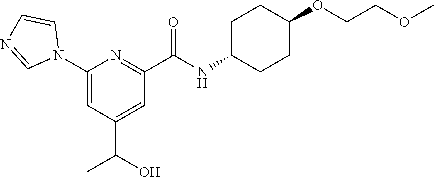 [SMILES: COCCO[C@H]1CC[C@H](NC(=O)c2cc(C(C)O)cc(-n3ccnc3)n2)CC1] | 4-(1-hydroxyethyl)-6-(1H- imidazol-1-yl)-N-((1r,4r)-4-(2- methoxyethoxy)cyclohexyl) picolinamide |
| X-135 | 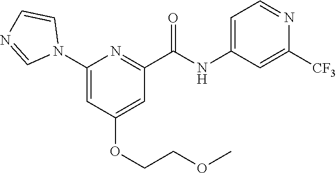 [SMILES: COCCOc1cc(C(=O)Nc2ccnc(C(F)(F)F)c2)nc(-n2ccnc2)c1] | 6-(1H-imidazol-1-yl)-4-(2- methoxyethoxy)-N-(2- (trifluoromethyl)pyridin-4- yl)picolinamide |
| X-136 | 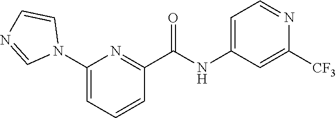 [SMILES: O=C(Nc1ccnc(C(F)(F)F)c1)c1cccc(-n2ccnc2)n1] | 6-(1H-imidazol-1-yl)-N-(2- (trifluoromethyl)pyridin-4- yl)picolinamide |
| X-137 | 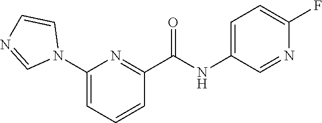 [SMILES: O=C(Nc1ccc(F)nc1)c1cccc(-n2ccnc2)n1] | N-(6-fluoropyridin-3-yl)-6-(1H- imidazol-1-yl)picolinamide |
| X-138 | 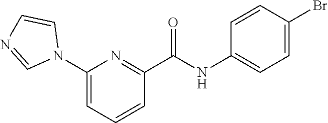 [SMILES: O=C(Nc1ccc(Br)cc1)c1cccc(-n2ccnc2)n1] | N-(4-bromophenyl)-6-(1H- imidazol-1-yl)picolinamide |
| X-139 | 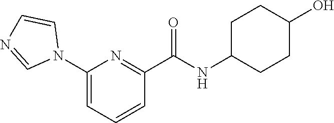 [SMILES: O=C(NC1CCC(O)CC1)c1cccc(-n2ccnc2)n1] | N-(4-hydroxycyclohexyl)-6-(1H- imidazol-1-yl)picolinamide |
| X-140 | 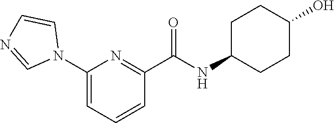 [SMILES: O=C(N[C@H]1CC[C@H](O)CC1)c1cccc(-n2ccnc2)n1] | N-((1r,4r)-4-hydroxycyclohexyl)-6- (1H-imidazol-1-yl)picolinamide |
| X-141 | 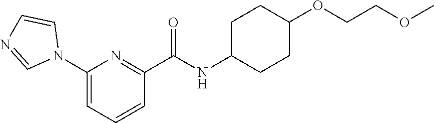 [SMILES: COCCOC1CCC(NC(=O)c2cccc(-n3ccnc3)n2)CC1] | 6-(1H-imidazol-1-yl)-N-(4-(2- methoxyethoxy)cyclohexyl) picolinamide |
| X-142 | 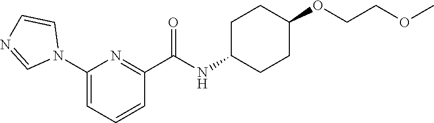 [SMILES: COCCO[C@H]1CC[C@H](NC(=O)c2cccc(-n3ccnc3)n2)CC1] | 6-(1H-imidazol-1-yl)-N-((1r,4r)-4-(2- methoxyethoxy)cyclohexyl) picolinamide |
| X-143 | 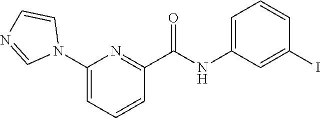 [SMILES: O=C(Nc1cccc(I)c1)c1cccc(-n2ccnc2)n1] | 6-(1H-imidazol-1-yl)-N-(3- iodophenyl)picolinamide |
| X-144 | 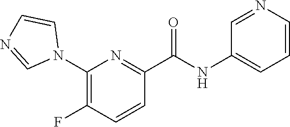 [SMILES: O=C(Nc1cccnc1)c1ccc(F)c(-n2ccnc2)n1] | 5-fluoro-6-(1H-imidazol-1-yl)-N- (pyridin-3-yl)picolinamide |
| X-145 | 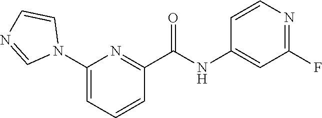 [SMILES: O=C(Nc1ccnc(F)c1)c1cccc(-n2ccnc2)n1] | N-(2-fluoropyridin-4-yl)-6-(1H- imidazol-1-yl)picolinamide |
| X-146 | 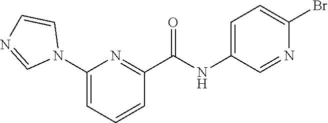 [SMILES: O=C(Nc1ccc(Br)nc1)c1cccc(-n2ccnc2)n1] | N-(6-bromopyridin-3-yl)-6-(1H- imidazol-1-yl)picolinamide |
| X-147 | 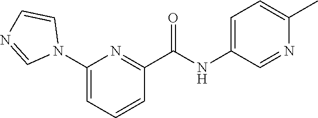 [SMILES: Cc1ccc(NC(=O)c2cccc(-n3ccnc3)n2)cn1] | 6-(1H-imidazol-1-yl)-N-(6- methylpyridin-3-yl)picolinamide |
| X-148 | 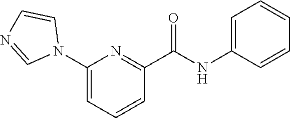 [SMILES: O=C(Nc1ccccc1)c1cccc(-n2ccnc2)n1] | 6-(1H-imidazol-1-yl)-N- phenylpicolinamide |
| X-149 | 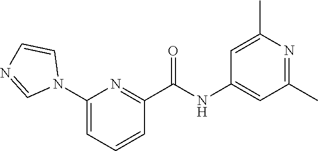 [SMILES: Cc1cc(NC(=O)c2cccc(-n3ccnc3)n2)cc(C)n1] | N-(2,6-dimethylpyridin-4-yl)-6- (1H-imidazol-1-yl)picolinamide |
| X-150 | 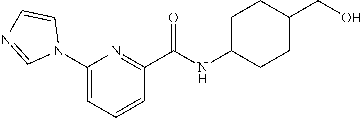 [SMILES: O=C(NC1CCC(CO)CC1)c1cccc(-n2ccnc2)n1] | N-(4-(hydroxymethyl)cyclohexyl)- 6-(1H-imidazol-1-yl)picolinamide |
| X-151 | 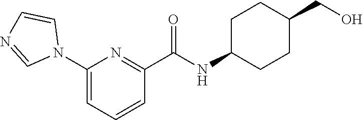 [SMILES: O=C(N[C@H]1CC[C@@H](CO)CC1)c1cccc(-n2ccnc2)n1] | N-((1s,4s)-4- (hydroxymethyl)cyclohexyl)-6- (1H-imidazol-1-yl)picolinamide |
| X-152 | 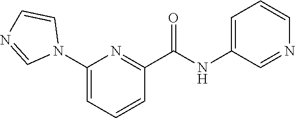 [SMILES: O=C(Nc1cccnc1)c1cccc(-n2ccnc2)n1] | 6-(1H-imidazol-1-yl)-N-(pyridin-3- yl)picolinamide |
| X-153 | 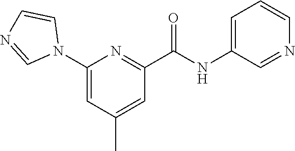 [SMILES: Cc1cc(C(=O)Nc2cccnc2)nc(-n2ccnc2)c1] | 6-(1H-imidazol-1-yl)-4-methyl-N- (pyridin-3-yl)picolinamide |
| X-154 | 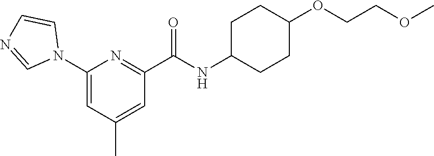 [SMILES: COCCOC1CCC(NC(=O)c2cc(C)cc(-n3ccnc3)n2)CC1] | 6-(1H-imidazol-1-yl)-N-(4-(2- methoxyethoxy)cyclohexyl)-4- methylpicolinamide |
| X-155 | 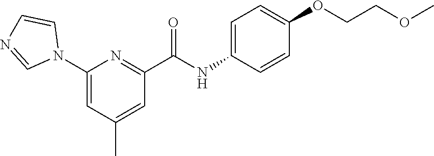 | 6-(1H-imidazol-1-yl)-N-((1r,4r)-4- (2-methoxyethoxy)cyclohexyl)-4- methylpicolinamide |
| X-156 | 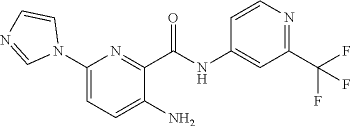 [SMILES: Nc1ccc(-n2ccnc2)nc1C(=O)Nc1ccnc(C(F)(F)F)c1] | 3-amino-6-(1H-imidazol-1-yl)-N- (2-(trifluoromethyl)pyridin-4- yl)picolinamide |
| X-157 | 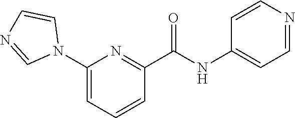 [SMILES: O=C(Nc1ccncc1)c1cccc(-n2ccnc2)n1] | 6-(1H-imidazol-1-yl)-N-(pyridin-4- yl)picolinamide |
| X-158 | 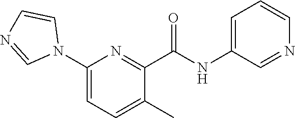 [SMILES: Cc1ccc(-n2ccnc2)nc1C(=O)Nc1cccnc1] | 6-(1H-imidazol-1-yl)-3-methyl-N- (pyridin-3-yl)picolinamide |
| X-159 | 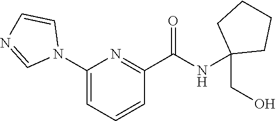 [SMILES: O=C(NC1(CO)CCCC1)c1cccc(-n2ccnc2)n1] | N-(1-(hydroxymethyl)cyclopentyl)- 6-(1H-imidazol-1-yl)picolinamide |
| X-160 | 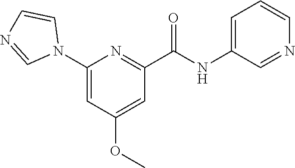 [SMILES: COc1cc(C(=O)Nc2cccnc2)nc(-n2ccnc2)c1] | 6-(1H-imidazol-1-yl)-4-methoxy- N-(pyridin-3-yl)picolinamide |
| X-161 | 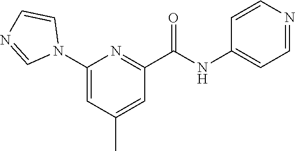 [SMILES: Cc1cc(C(=O)Nc2ccncc2)nc(-n2ccnc2)c1] | 6-(1H-imidazol-1-yl)-4-methyl-N- (pyridin-4-yl)picolinamide |
| X-162 | 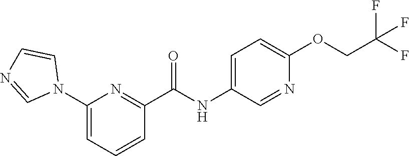 [SMILES: O=C(Nc1ccc(OCC(F)(F)F)nc1)c1cccc(-n2ccnc2)n1] | 6-(1H-imidazol-1-yl)-N-(6-(2,2,2- trifluoroethoxy)pyridin-3- yl)picolinamide |
| X-163 | 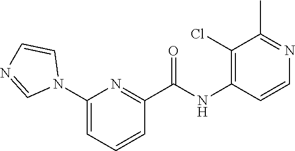 [SMILES: Cc1nccc(NC(=O)c2cccc(-n3ccnc3)n2)c1Cl] | N-(3-chloro-2-methylpyridin-4-yl)- 6-(1H-imidazol-1-yl)picolinamide |
| X-164 | 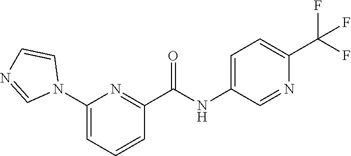 [SMILES: O=C(Nc1ccc(C(F)(F)F)nc1)c1cccc(-n2ccnc2)n1] | 6-(1H-imidazol-1-yl)-N-(6- (trifluoromethyl)pyridin-3- yl)picolinamide |
| X-165 | 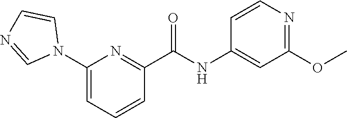 [SMILES: COc1cc(NC(=O)c2cccc(-n3ccnc3)n2)ccn1] | 6-(1H-imidazol-1-yl)-N-(2- methoxypyridin-4-yl)picolinamide |
| X-166 | 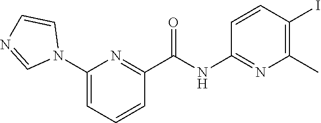 [SMILES: Cc1nc(NC(=O)c2cccc(-n3ccnc3)n2)ccc1I] | 6-(1H-imidazol-1-yl)-N-(5-iodo-6- methylpyridin-2-yl)picolinamide |
| X-167 | 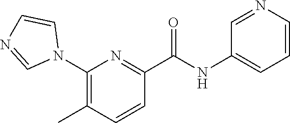 [SMILES: Cc1ccc(C(=O)Nc2cccnc2)nc1-n1ccnc1] | 6-(1H-imidazol-1-yl)-5-methyl-N- (pyridin-3-yl)picolinamide |
| X-168 | 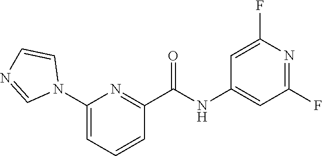 [SMILES: O=C(Nc1cc(F)nc(F)c1)c1cccc(-n2ccnc2)n1] | N-(2,6-difluoropyridin-4-yl)-6-(1H- imidazol-1-yl)picolinamide |
| X-169 | 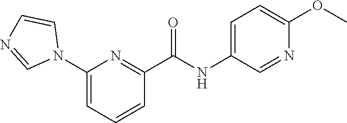 [SMILES: COc1ccc(NC(=O)c2cccc(-n3ccnc3)n2)cn1] | 6-(1H-imidazol-1-yl)-N-(6- methoxypyridin-3-yl)picolinamide |
| X-170 | 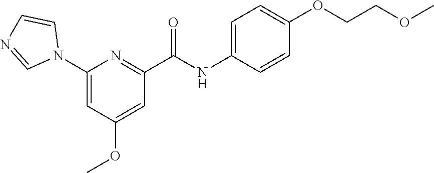 [SMILES: COCCOc1ccc(NC(=O)c2cc(OC)cc(-n3ccnc3)n2)cc1] | 6-(1H-imidazol-1-yl)-4-methoxy- N-(4-(2- methoxyethoxy)cyclohexyl) picolinamide |
| X-171 | 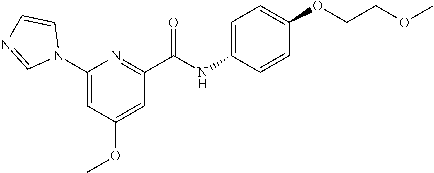 | 6-(1H-imidazol-1-yl)-4-methoxy- N-((1r,4r)-4-(2- methoxyethoxy)cyclohexyl) picolinamide |
| X-172 | 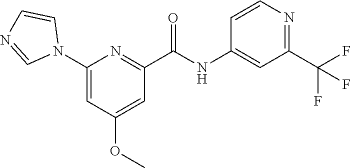 [SMILES: COc1cc(C(=O)Nc2ccnc(C(F)(F)F)c2)nc(-n2ccnc2)c1] | 6-(1H-imidazol-1-yl)-4-methoxy- N-(2-(trifluoromethyl)pyridin-4- yl)picolinamide |
| X-173 | 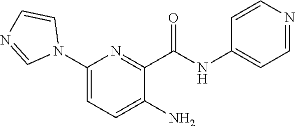 [SMILES: Nc1ccc(-n2ccnc2)nc1C(=O)Nc1ccncc1] | 3-amino-6-(1H-imidazol-1-yl)-N- (pyridin-4-yl)picolinamide |
| X-174 | 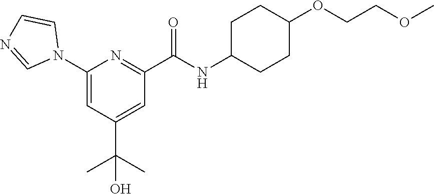 [SMILES: COCCOC1CCC(NC(=O)c2cc(C(C)(C)O)cc(-n3ccnc3)n2)CC1] | 4-(2-hydroxypropan-2-yl)-6-(1H- imidazol-1-yl)-N-(4-(2- methoxyethoxy)cyclohexyl) picolinamide |
| X-175 | 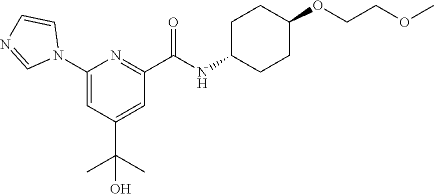 [SMILES: COCCO[C@H]1CC[C@H](NC(=O)c2cc(C(C)(C)O)cc(-n3ccnc3)n2)CC1] | 4-(2-hydroxypropan-2-yl)-6-(1H- imidazol-1-yl)-N-((1r,4r)-4-(2- methoxyethoxy)cyclohexyl) picolinamide |
| X-176 | 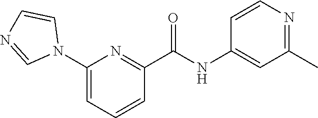 [SMILES: Cc1cc(NC(=O)c2cccc(-n3ccnc3)n2)ccn1] | 6-(1H-imidazol-1-yl)-N-(2- methylpyridin-4-yl)picolinamide |
| X-177 | 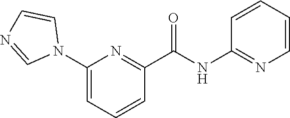 [SMILES: O=C(Nc1ccccn1)c1cccc(-n2ccnc2)n1] | 6-(1H-imidazol-1-yl)-N-(pyridin-2- yl)picolinamide |
| X-178 | 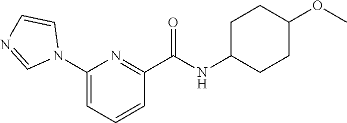 [SMILES: COC1CCC(NC(=O)c2cccc(-n3ccnc3)n2)CC1] | 6-(1H-imidazol-1-yl)-N-(4- methoxycyclohexyl)picolinamide |
| X-179 | 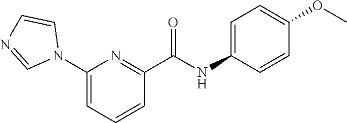 | 6-(1H-imidazol-1-yl)-N-((1r,4r)-4- methoxycyclohexyl)picolinamide |
| X-180 | 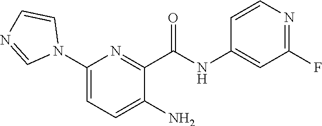 [SMILES: Nc1ccc(-n2ccnc2)nc1C(=O)Nc1ccnc(F)c1] | 3-amino-N-(2-fluoropyridin-4-yl)- 6-(1H-imidazol-1-yl)picolinamide |
| X-181 | 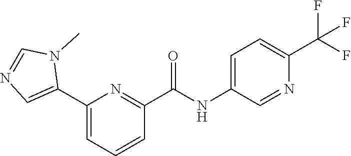 [SMILES: Cn1cncc1-c1cccc(C(=O)Nc2ccc(C(F)(F)F)nc2)n1] | 6-(1-methyl-1H-imidazol-5-yl)-N- (6-(trifluoromethyl)pyridin-3- yl)picolinamide |
| X-182 | 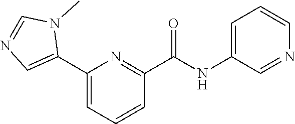 [SMILES: Cn1cncc1-c1cccc(C(=O)Nc2cccnc2)n1] | 6-(1-methyl-1H-imidazol-5-yl)-N- (pyridin-3-yl)picolinamide |
| X-183 | 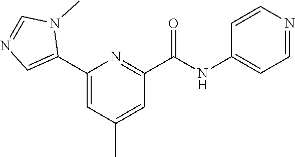 [SMILES: Cc1cc(C(=O)Nc2ccncc2)nc(-c2cncn2C)c1] | 4-methyl-6-(1-methyl-1H-imidazol- 5-yl)-N-(pyridin-4-yl)picolinamide |
| X-184 | 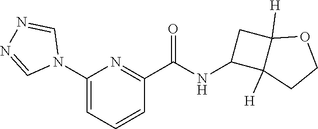 [SMILES: O=C(NC1CC2OCCC12)c1cccc(-n2cnnc2)n1] | N-(2-oxabicyclo[3.2.0]heptan-6- yl)-6-(4H-1,2,4-triazol-4- yl)picolinamide |
| X-185 | 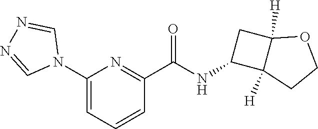 [SMILES: O=C(N[C@@H]1C[C@H]2OCC[C@@H]12)c1cccc(-n2cnnc2)n1] | N-((1R,5S,6R)-2- oxabicyclo[3.2.0]heptan-6-yl)-6- (4H-1,2,4-triazol-4-yl)picolinamide |
| X-186 | 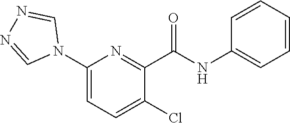 [SMILES: O=C(Nc1ccccc1)c1nc(-n2cnnc2)ccc1Cl] | 3-chloro-N-phenyl-6-(4H-1,2,4- triazol-4-yl)picolinamide |
| X-187 | 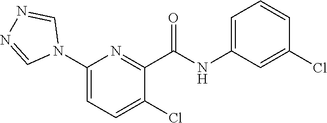 [SMILES: O=C(Nc1cccc(Cl)c1)c1nc(-n2cnnc2)ccc1Cl] | 3-chloro-N-(3-chlorophenyl)-6- (4H-1,2,4-triazol-4-yl)picolinamide |
| X-188 | 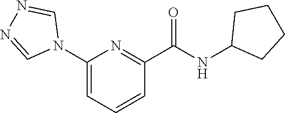 [SMILES: O=C(NC1CCCC1)c1cccc(-n2cnnc2)n1] | N-cyclopentyl-6-(4H-1,2,4-triazol- 4-yl)picolinamide |
| X-189 | 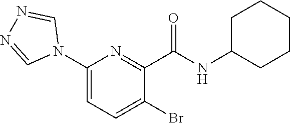 [SMILES: O=C(NC1CCCCC1)c1nc(-n2cnnc2)ccc1Br] | 3-bromo-N-cyclohexyl-6-(4H- 1,2,4-triazol-4-yl)picolinamide |
| X-190 | 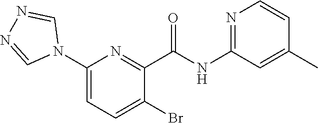 [SMILES: Cc1ccnc(NC(=O)c2nc(-n3cnnc3)ccc2Br)c1] | 3-bromo-N-(4-methylpyridin-2-yl)- 6-(4H-1,2,4-triazol-4- yl)picolinamide |
| X-191 | 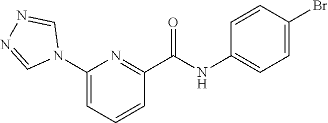 [SMILES: O=C(Nc1ccc(Br)cc1)c1cccc(-n2cnnc2)n1] | N-(4-bromophenyl)-6-(4H-1,2,4- triazol-4-yl)picolinamide |
| X-192 | 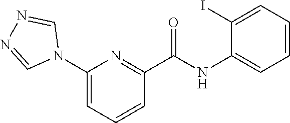 [SMILES: O=C(Nc1ccccc1I)c1cccc(-n2cnnc2)n1] | N-(2-iodophenyl)-6-(4H-1,2,4- triazol-4-yl)picolinamide |
| X-193 | 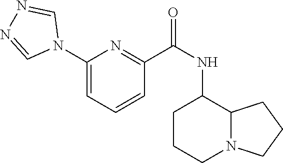 [SMILES: O=C(NC1CCCN2CCCC12)c1cccc(-n2cnnc2)n1] | N-(octahydroindolizin-8-yl)-6-(4H- 1,2,4-triazol-4-yl)picolinamide |
| X-194 | 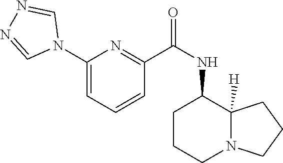 [SMILES: O=C(N[C@@H]1CCCN2CCC[C@H]12)c1cccc(-n2cnnc2)n1] | N-((8R,8aR)-octahydroindolizin-8- yl)-6-(4H-1,2,4-triazol-4- yl)picolinamide |
| X-195 | 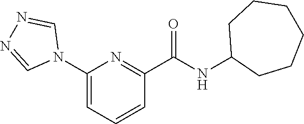 [SMILES: O=C(NC1CCCCCC1)c1cccc(-n2cnnc2)n1] | N-cycloheptyl-6-(4H-1,2,4-triazol- 4-yl)picolinamide |
| X-196 | 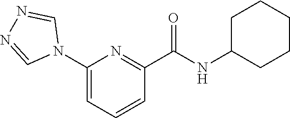 [SMILES: O=C(NC1CCCCC1)c1cccc(-n2cnnc2)n1] | N-cyclohexyl-6-(4H-1,2,4-triazol- 4-yl)picolinamide |
| X-197 | 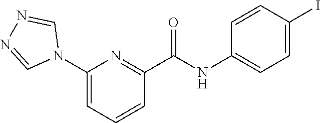 [SMILES: O=C(Nc1ccc(I)cc1)c1cccc(-n2cnnc2)n1] | N-(4-iodophenyl)-6-(4H-1,2,4- triazol-4-yl)picolinamide |
| X-198 | 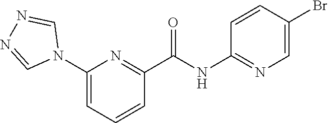 [SMILES: O=C(Nc1ccc(Br)cn1)c1cccc(-n2cnnc2)n1] | N-(5-bromopyridin-2-yl)-6-(4H- 1,2,4-triazol-4-yl)picolinamide |
| X-199 | 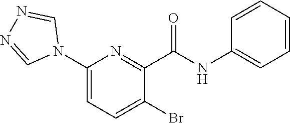 [SMILES: O=C(Nc1ccccc1)c1nc(-n2cnnc2)ccc1Br] | 3-bromo-N-phenyl-6-(4H-1,2,4- triazol-4-yl)picolinamide |
| X-200 | 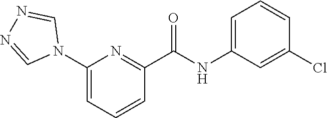 [SMILES: O=C(Nc1cccc(Cl)c1)c1cccc(-n2cnnc2)n1] | N-(3-chlorophenyl)-6-(4H-1,2,4- triazol-4-yl)picolinamide |
| X-201 | 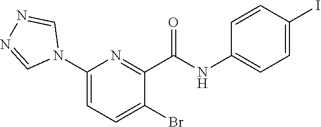 [SMILES: O=C(Nc1ccc(I)cc1)c1nc(-n2cnnc2)ccc1Br] | 3-bromo-N-(4-iodophenyl)-6-(4H- 1,2,4-triazol-4-yl)picolinamide |
| X-202 | 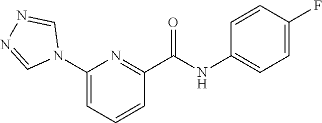 [SMILES: O=C(Nc1ccc(F)cc1)c1cccc(-n2cnnc2)n1] | N-(4-fluorophenyl)-6-(4H-1,2,4- triazol-4-yl)picolinamide |
| X-203 | 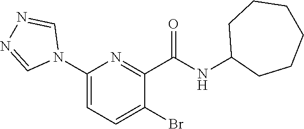 [SMILES: O=C(NC1CCCCCC1)c1nc(-n2cnnc2)ccc1Br] | 3-bromo-N-cycloheptyl-6-(4H- 1,2,4-triazol-4-yl)picolinamide |
| X-204 | 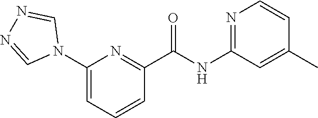 [SMILES: Cc1ccnc(NC(=O)c2cccc(-n3cnnc3)n2)c1] | N-(4-methylpyridin-2-yl)-6-(4H- 1,2,4-triazol-4-yl)picolinamide |
| X-205 | 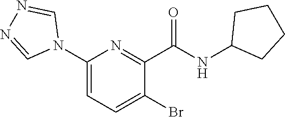 [SMILES: O=C(NC1CCCC1)c1nc(-n2cnnc2)ccc1Br] | 3-bromo-N-cyclopentyl-6-(4H- 1,2,4-triazol-4-yl)picolinamide |
| X-206 | 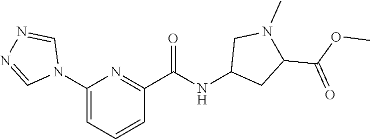 [SMILES: COC(=O)C1CC(NC(=O)c2cccc(-n3cnnc3)n2)CN1C] | methyl 4-(6-(4H-1,2,4-triazol-4- yl)picolinamido)-1- methylpyrrolidine-2-carboxylate |
| X-207 | 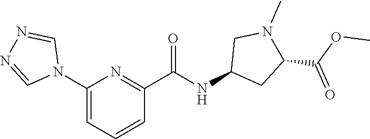 [SMILES: COC(=O)[C@@H]1C[C@@H](NC(=O)c2cccc(-n3cnnc3)n2)CN1C] | methyl (2S,4R)-4-(6-(4H-1,2,4- triazol-4-yl)picolinamido)-1- methylpyrrolidine-2-carboxylate |
| X-208 | 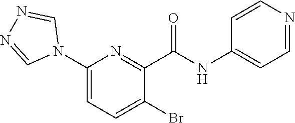 [SMILES: O=C(Nc1ccncc1)c1nc(-n2cnnc2)ccc1Br] | 3-bromo-N-(pyridin-4-yl)-6-(4H- 1,2,4-triazol-4-yl)picolinamide |
| X-209 | 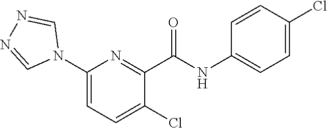 [SMILES: O=C(Nc1ccc(Cl)cc1)c1nc(-n2cnnc2)ccc1Cl] | 3-chloro-N-(4-chlorophenyl)-6- (4H-1,2,4-triazol-4-yl)picolinamide |
| X-210 | 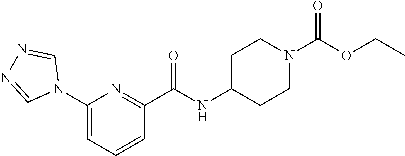 [SMILES: CCOC(=O)N1CCC(NC(=O)c2cccc(-n3cnnc3)n2)CC1] | ethyl 4-(6-(4H-1,2,4-triazol-4- yl)picolinamido)piperidine-1- carboxylate |
| X-211 | 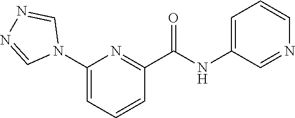 [SMILES: O=C(Nc1cccnc1)c1cccc(-n2cnnc2)n1] | N-(pyridin-3-yl)-6-(4H-1,2,4- triazol-4-yl)picolinamide |
| X-212 | 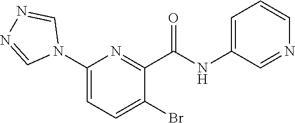 [SMILES: O=C(Nc1cccnc1)c1nc(-n2cnnc2)ccc1Br] | 3-bromo-N-(pyridin-3-yl)-6-(4H- 1,2,4-triazol-4-yl)picolinamide |
| X-213 | 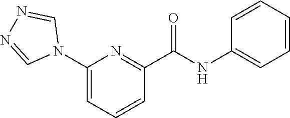 [SMILES: O=C(Nc1ccccc1)c1cccc(-n2cnnc2)n1] | N-phenyl-6-(4H-1,2,4-triazol-4- yl)picolinamide |
| X-214 | 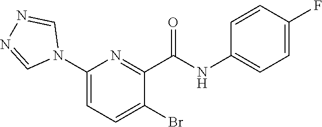 [SMILES: O=C(Nc1ccc(F)cc1)c1nc(-n2cnnc2)ccc1Br] | 3-bromo-N-(4-fluorophenyl)-6- (4H-1,2,4-triazol-4-yl)picolinamide |
| X-215 | 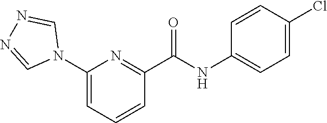 [SMILES: O=C(Nc1ccc(Cl)cc1)c1cccc(-n2cnnc2)n1] | N-(4-chlorophenyl)-6-(4H-1,2,4- triazol-4-yl)picolinamide |
| X-216 | 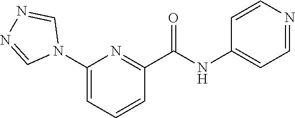 [SMILES: O=C(Nc1ccncc1)c1cccc(-n2cnnc2)n1] | N-(pyridin-4-yl)-6-(4H-1,2,4- triazol-4-yl)picolinamide |
| X-217 | 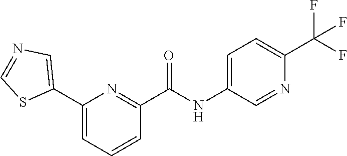 [SMILES: O=C(Nc1ccc(C(F)(F)F)nc1)c1cccc(-c2cncs2)n1] | 6-(thiazol-5-yl)-N-(6- (trifluoromethyl)pyridin-3- yl)picolinamide |
| X-218 | 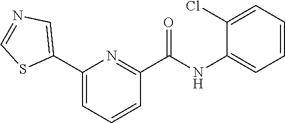 [SMILES: O=C(Nc1ccccc1Cl)c1cccc(-c2cncs2)n1] | N-(2-chlorophenyl)-6-(thiazol-5- yl)picolinamide |
| X-219 | 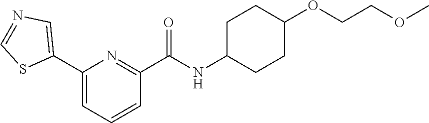 [SMILES: COCCOC1CCC(NC(=O)c2cccc(-c3cncs3)n2)CC1] | N-(4-(2- methoxyethoxy)cyclohexyl)-6- (thiazol-5-yl)picolinamide |
| X-220 | 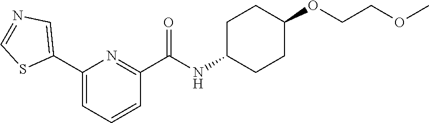 [SMILES: COCCO[C@H]1CC[C@H](NC(=O)c2cccc(-c3cncs3)n2)CC1] | N-((1r,4r)-4-(2- methoxyethoxy)cyclohexyl)-6- (thiazol-5-yl)picolinamide |
| X-221 | 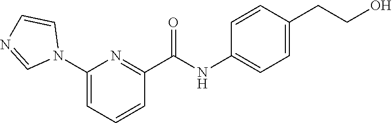 [SMILES: O=C(Nc1ccc(CCO)cc1)c1cccc(-n2ccnc2)n1] | N-(4-(2-hydroxyethyl)phenyl)-6- (1H-imidazol-1-yl)picolinamide |

TABLE 3X

| Compound | | |
|---|---|---|
| Number | Structure | Name |
| X-222 | 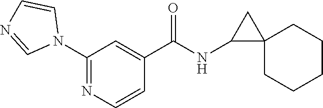 [SMILES: O=C(NC1CC12CCCCC2)c1ccnc(-n2ccnc2)c1] | 2-(1H-imidazol-1-yl)-N- (spiro[2.5]octan-1- yl)isonicotinamide |
| X-223 | 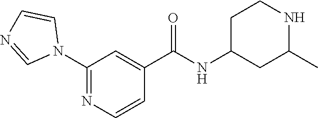 [SMILES: CC1CC(NC(=O)c2ccnc(-n3ccnc3)c2)CCN1] | 2-(1H-imidazol-1-yl)-N-(2- methylpiperidin-4- yl)isonicotinamide |
| X-224 | 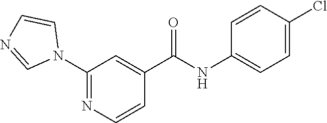 [SMILES: O=C(Nc1ccc(Cl)cc1)c1ccnc(-n2ccnc2)c1] | N-(4-chlorophenyl)-2-(1H- imidazol-1-yl)isonicotinamide |
| X-225 | 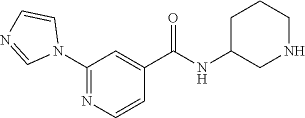 [SMILES: O=C(NC1CCCNC1)c1ccnc(-n2ccnc2)c1] | 2-(1H-imidazol-1-yl)-N-(piperidin- 3-yl)isonicotinamide |
| X-226 | 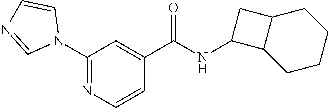 [SMILES: O=C(NC1CC2CCCCC21)c1ccnc(-n2ccnc2)c1] | N-(bicyclo[4.2.0]octan-7-yl)-2- (1H-imidazol-1-yl)isonicotinamide |
| X-227 | 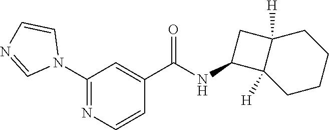 [SMILES: O=C(N[C@H]1C[C@H]2CCCC[C@H]21)c1ccnc(-n2ccnc2)c1] | N-((1R,6R,7S)-bicyclo[4.2.0]octan- 7-yl)-2-(1H-imidazol-1- yl)isonicotinamide |
| X-228 | 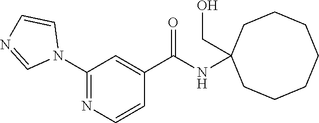 [SMILES: O=C(NC1(CO)CCCCCCC1)c1ccnc(-n2ccnc2)c1] | N-(1-(hydroxymethyl)cyclooctyl)- 2-(1H-imidazol-1- yl)isonicotinamide |
| X-229 | 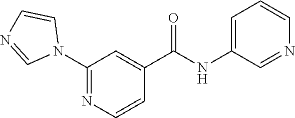 [SMILES: O=C(Nc1cccnc1)c1ccnc(-n2ccnc2)c1] | 2-(1H-imidazol-1-yl)-N-(pyridin-3- yl)isonicotinamide |
| X-230 | 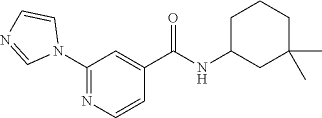 [SMILES: CC1(C)CCCC(NC(=O)c2ccnc(-n3ccnc3)c2)C1] | N-(3,3-dimethylcyclohexyl)-2-(1H- imidazol-1-yl)isonicotinamide |
| X-231 | 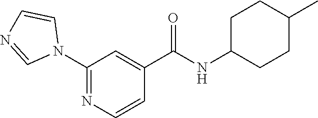 [SMILES: CC1CCC(NC(=O)c2ccnc(-n3ccnc3)c2)CC1] | 2-(1H-imidazol-1-yl)-N-(4- methylcyclohexyl)isonicotinamide |
| X-232 | 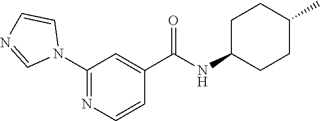 [SMILES: C[C@H]1CC[C@H](NC(=O)c2ccnc(-n3ccnc3)c2)CC1] | 2-(1H-imidazol-1-yl)-N-((1r,4r)-4- methylcyclohexyl)isonicotinamide |
| X-233 | 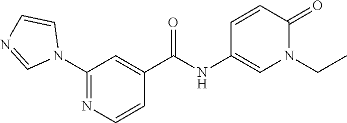 [SMILES: CCn1cc(NC(=O)c2ccnc(-n3ccnc3)c2)ccc1=O] | N-(1-ethyl-6-oxo-1,6- dihydropyridin-3-yl)-2-(1H- imidazol-1-yl)isonicotinamide |
| X-234 | 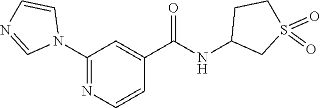 [SMILES: O=C(NC1CCS(=O)(=O)C1)c1ccnc(-n2ccnc2)c1] | N-(1,1-dioxidotetrahydrothiophen- 3-yl)-2-(1H-imidazol-1- yl)isonicotinamide |
| X-235 | 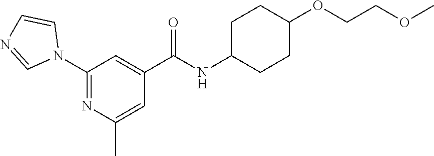 [SMILES: COCCOC1CCC(NC(=O)c2cc(C)nc(-n3ccnc3)c2)CC1] | 2-(1H-imidazol-1-yl)-N-(4-(2- methoxyethoxy)cyclohexyl)-6- methylisonicotinamide |
| X-236 | 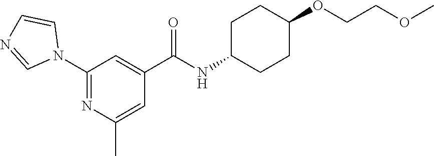 [SMILES: COCCO[C@H]1CC[C@H](NC(=O)c2cc(C)nc(-n3ccnc3)c2)CC1] | 2-(1H-imidazol-1-yl)-N-((1r,4r)-4- (2-methoxyethoxy)cyclohexyl)-6- methylisonicotinamide |
| X-237 | 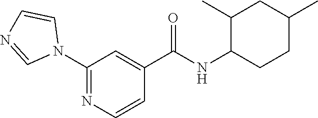 [SMILES: CC1CCC(NC(=O)c2ccnc(-n3ccnc3)c2)C(C)C1] | N-(2,4-dimethylcyclohexyl)-2-(1H- imidazol-1-yl)isonicotinamide |
| X-238 | 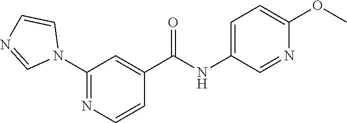 [SMILES: COc1ccc(NC(=O)c2ccnc(-n3ccnc3)c2)cn1] | 2-(1H-imidazol-1-yl)-N-(6- methoxypyridin-3- yl)isonicotinamide |
| X-239 | 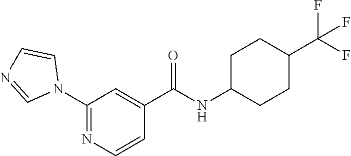 [SMILES: O=C(NC1CCC(C(F)(F)F)CC1)c1ccnc(-n2ccnc2)c1] | 2-(1H-imidazol-1-yl)-N-(4- (trifluoromethyl)cyclohexyl) isonicotinamide |
| X-240 | 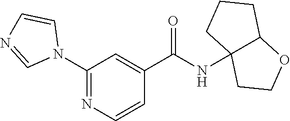 [SMILES: O=C(NC12CCCC1OCC2)c1ccnc(-n2ccnc2)c1] | N-(hexahydro-3aH- cyclopenta[b]furan-3a-yl)-2-(1H- imidazol-1-yl)isonicotinamide |
| X-241 | 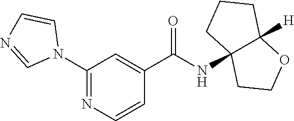 [SMILES: O=C(N[C@@]12CCC[C@@H]1OCC2)c1ccnc(-n2ccnc2)c1] | N-((3aR,6aS)-hexahydro-3aH- cyclopenta[b]furan-3a-yl)-2-(1H- imidazol-1-yl)isonicotinamide |
| X-242 | 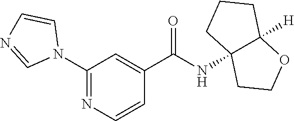 [SMILES: O=C(N[C@]12CCC[C@H]1OCC2)c1ccnc(-n2ccnc2)c1] | N-((3aS,6aR)-hexahydro-3aH- cyclopenta[b]furan-3a-yl)-2-(1H- imidazol-1-yl)isonicotinamide |
| X-243 | 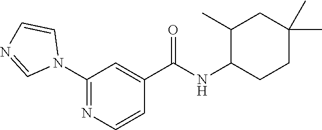 [SMILES: CC1CC(C)(C)CCC1NC(=O)c1ccnc(-n2ccnc2)c1] | 2-(1H-imidazol-1-yl)-N-(2,4,4- trimethylcyclohexyl)isonicotinamide |
| X-244 | 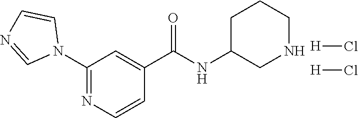 | 2-(1H-imidazol-1-yl)-N-(piperidin- 3-yl)isonicotinamide dihydrochloride |
| X-245 | 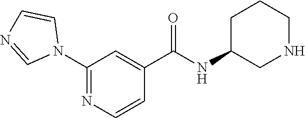 [SMILES: O=C(N[C@H]1CCCNC1)c1ccnc(-n2ccnc2)c1] | (S)-2-(1H-imidazol-1-yl)-N- (piperidin-3-yl)isonicotinamide |
| X-246 | 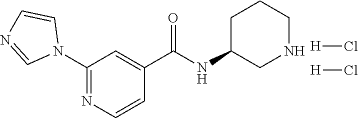 | (S)-2-(1H-imidazol-1-yl)-N- (piperidin-3-yl)isonicotinamide dihydrochloride |
| X-247 | 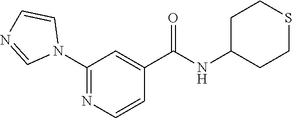 [SMILES: O=C(NC1CCSCC1)c1ccnc(-n2ccnc2)c1] | 2-(1H-imidazol-1-yl)-N- (tetrahydro-2H-thiopyran-4- yl)isonicotinamide |
| X-248 | 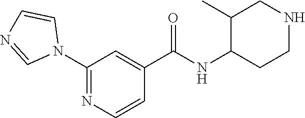 [SMILES: CC1CNCCC1NC(=O)c1ccnc(-n2ccnc2)c1] | 2-(1H-imidazol-1-yl)-N-(3- methylpiperidin-4- yl)isonicotinamide |
| X-249 | 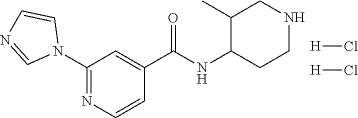 | 2-(1H-imidazol-1-yl)-N-(3- methylpiperidin-4- yl)isonicotinamide dihydrochloride |
| X-250 | 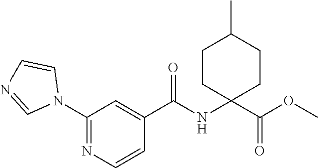 [SMILES: COC(=O)C1(NC(=O)c2ccnc(-n3ccnc3)c2)CCC(C)CC1] | methyl 1-(2-(1H-imidazol-1- yl)isonicotinamido)-4- methylcyclohexane-1-carboxylate |
| X-251 | 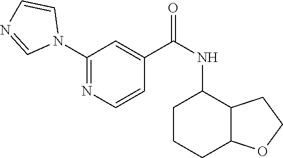 [SMILES: O=C(NC1CCCC2OCCC12)c1ccnc(-n2ccnc2)c1] | 2-(1H-imidazol-1-yl)-N- (octahydrobenzofuran-4- yl)isonicotinamide |
| X-252 | 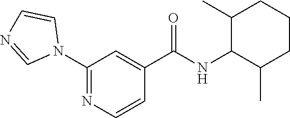 [SMILES: CC1CCCC(C)C1NC(=O)c1ccnc(-n2ccnc2)c1] | N-(2,6-dimethylcyclohexyl)-2-(1H- imidazol-1-yl)isonicotinamide |
| X-253 | 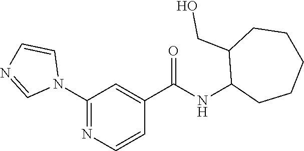 [SMILES: O=C(NC1CCCCCC1CO)c1ccnc(-n2ccnc2)c1] | N-(2-(hydroxymethyl)cycloheptyl)- 2-(1H-imidazol-1- yl)isonicotinamide |
| X-254 | 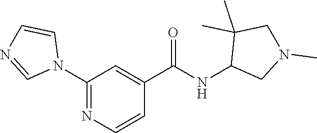 [SMILES: CN1CC(NC(=O)c2ccnc(-n3ccnc3)c2)C(C)(C)C1] | 2-(1H-imidazol-1-yl)-N-(1,4,4- trimethylpyrrolidin-3- yl)isonicotinamide |
| X-255 | 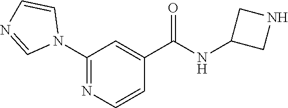 [SMILES: O=C(NC1CNC1)c1ccnc(-n2ccnc2)c1] | N-(azetidin-3-yl)-2-(1H-imidazol- 1-yl)isonicotinamide |
| X-256 | 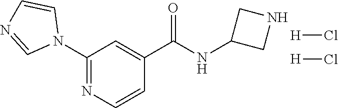 | N-(azetidin-3-yl)-2-(1H-imidazol- 1-yl)isonicotinamide dihydrochloride |
| X-257 | 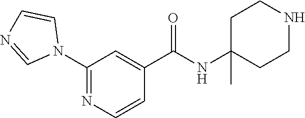 [SMILES: CC1(NC(=O)c2ccnc(-n3ccnc3)c2)CCNCC1] | 2-(1H-imidazol-1-yl)-N-(4- methylpiperidin-4- yl)isonicotinamide |
| X-258 | 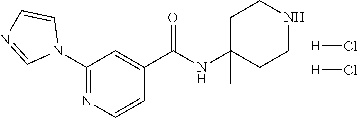 | 2-(1H-imidazol-1-yl)-N-(4- methylpiperidin-4- yl)isonicotinamide dihydrochloride |
| X-259 | 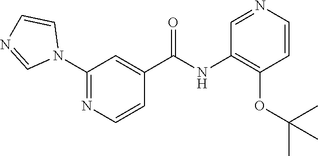 [SMILES: CC(C)(C)Oc1ccncc1NC(=O)c1ccnc(-n2ccnc2)c1] | N-(4-(tert-butoxy)pyridin-3-yl)-2- (1H-imidazol-1-yl)isonicotinamide |
| X-260 | 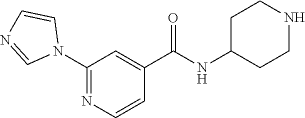 [SMILES: O=C(NC1CCNCC1)c1ccnc(-n2ccnc2)c1] | 2-(1H-imidazol-1-yl)-N-(piperidin- 4-yl)isonicotinamide |
| X-261 | 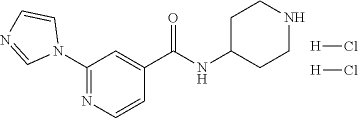 | 2-(1H-imidazol-1-yl)-N-(piperidin- 4-yl)isonicotinamide dihydrochloride |
| X-262 | 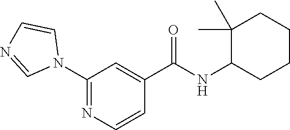 [SMILES: CC1(C)CCCCC1NC(=O)c1ccnc(-n2ccnc2)c1] | N-(2,2-dimethylcyclohexyl)-2-(1H- imidazol-1-yl)isonicotinamide |
| X-263 | 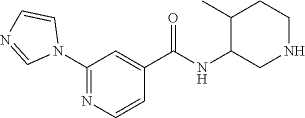 [SMILES: CC1CCNCC1NC(=O)c1ccnc(-n2ccnc2)c1] | 2-(1H-imidazol-1-yl)-N-(4- methylpiperidin-3- yl)isonicotinamide |
| X-264 | 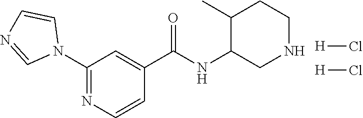 | 2-(1H-imidazol-1-yl)-N-(4- methylpiperidin-3- yl)isonicotinamide dihydrochloride |
| X-265 | 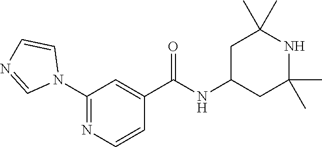 [SMILES: CC1(C)CC(NC(=O)c2ccnc(-n3ccnc3)c2)CC(C)(C)N1] | 2-(1H-imidazol-1-yl)-N-(2,2,6,6- tetramethylpiperidin-4- yl)isonicotinamide |
| X-266 | 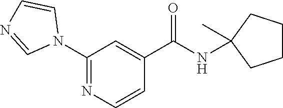 [SMILES: CC1(NC(=O)c2ccnc(-n3ccnc3)c2)CCCC1] | 2-(1H-imidazol-1-yl)-N-(1- methylcyclopentyl)isonicotinamide |
| X-267 | 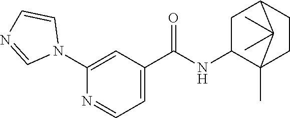 [SMILES: CC1(C)C2CCC1(C)C(NC(=O)c1ccnc(-n3ccnc3)c1)C2] | 2-(1H-imidazol-1-yl)-N-(1,7,7- trimethylbicyclo[2.2.1]heptan-2- yl)isonicotinamide |
| X-268 | 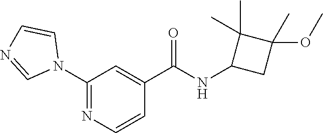 [SMILES: COC1(C)CC(NC(=O)c2ccnc(-n3ccnc3)c2)C1(C)C] | 2-(1H-imidazol-1-yl)-N-(3- methoxy-2,2,3- trimethylcyclobutyl)isonicotinamide |
| X-269 | 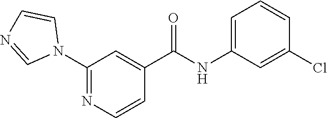 [SMILES: O=C(Nc1cccc(Cl)c1)c1ccnc(-n2ccnc2)c1] | N-(3-chlorophenyl)-2-(1H- imidazol-1-yl)isonicotinamide |
| X-270 | 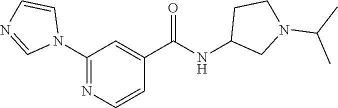 [SMILES: CC(C)N1CCC(NC(=O)c2ccnc(-n3ccnc3)c2)C1] | 2-(1H-imidazol-1-yl)-N-(1- isopropylpyrrolidin-3- yl)isonicotinamide |
| X-271 | 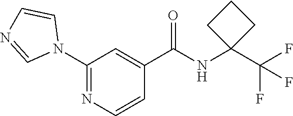 [SMILES: O=C(NC1(C(F)(F)F)CCC1)c1ccnc(-n2ccnc2)c1] | 2-(1H-imidazol-1-yl)-N-(1- (trifluoromethyl)cyclobutyl) isonicotinamide |
| X-272 | 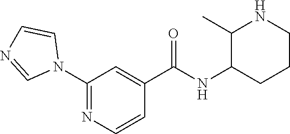 [SMILES: CC1NCCCC1NC(=O)c1ccnc(-n2ccnc2)c1] | 2-(1H-imidazol-1-yl)-N-(2- methylpiperidin-3- yl)isonicotinamide |
| X-273 | 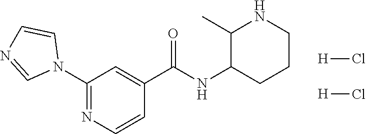 | 2-(1H-imidazol-1-yl)-N-(2- methylpiperidin-3- yl)isonicotinamide dihydrochloride |
| X-274 | 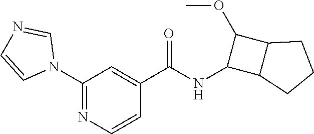 [SMILES: COC1C2CCCC2C1NC(=O)c1ccnc(-n2ccnc2)c1] | 2-(1H-imidazol-1-yl)-N-(7- methoxybicyclo[3.2.0]heptan-6- yl)isonicotinamide |
| X-275 | 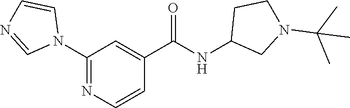 [SMILES: CC(C)(C)N1CCC(NC(=O)c2ccnc(-n3ccnc3)c2)C1] | N-(1-(tert-butyl)pyrrolidin-3-yl)-2- (1H-imidazol-1-yl)isonicotinamide |
| X-276 | 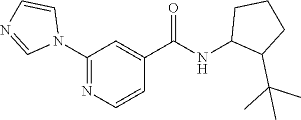 [SMILES: CC(C)(C)C1CCCC1NC(=O)c1ccnc(-n2ccnc2)c1] | N-(2-(tert-butyl)cyclopentyl)-2- (1H-imidazol-1-yl)isonicotinamide |
| X-277 | 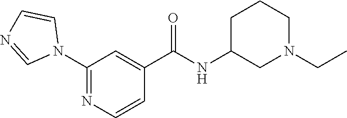 [SMILES: CCN1CCCC(NC(=O)c2ccnc(-n3ccnc3)c2)C1] | N-(1-ethylpiperidin-3-yl)-2-(1H- imidazol-1-yl)isonicotinamide |
| X-278 | 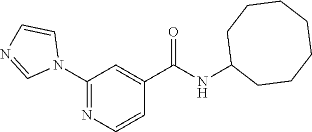 [SMILES: O=C(NC1CCCCCCC1)c1ccnc(-n2ccnc2)c1] | N-cyclooctyl-2-(1H-imidazol-1- yl)isonicotinamide |
| X-279 | 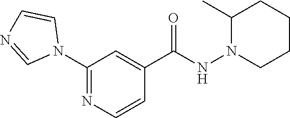 [SMILES: CC1CCCCN1NC(=O)c1ccnc(-n2ccnc2)c1] | 2-(1H-imidazol-1-yl)-N-(2- methylpiperidin-1- yl)isonicotinamide |
| X-280 | 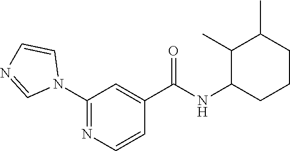 [SMILES: CC1CCCC(NC(=O)c2ccnc(-n3ccnc3)c2)C1C] | N-(2,3-dimethylcyclohexyl)-2-(1H- imidazol-1-yl)isonicotinamide |
| X-281 | 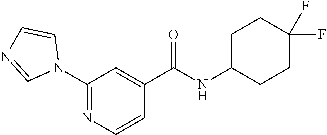 [SMILES: O=C(NC1CCC(F)(F)CC1)c1ccnc(-n2ccnc2)c1] | N-(4,4-difluorocyclohexyl)-2-(1H- imidazol-1-yl)isonicotinamide |
| X-282 | 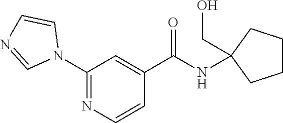 [SMILES: O=C(NC1(CO)CCCC1)c1ccnc(-n2ccnc2)c1] | N-(1-(hydroxymethyl)cyclopentyl)- 2-(1H-imidazol-1- yl)isonicotinamide |
| X-283 | 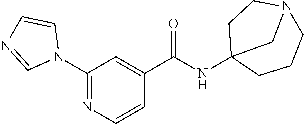 [SMILES: O=C(NC12CCCN(CC1)C2)c1ccnc(-n2ccnc2)c1] | N-(1-azabicyclo[3.2.1]octan-5-yl)- 2-(1H-imidazol-1- yl)isonicotinamide |
| X-284 | 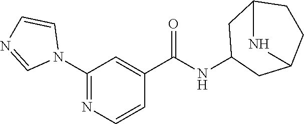 [SMILES: O=C(NC1CC2CCC(C1)N2)c1ccnc(-n2ccnc2)c1] | N-(8-azabicyclo[3.2.1]octan-3-yl)- 2-(1H-imidazol-1- yl)isonicotinamide |
| X-285 | 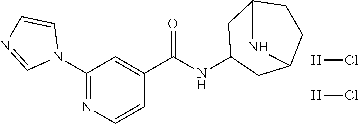 | N-(8-azabicyclo[3.2.1]octan-3-yl)- 2-(1H-imidazol-1- yl)isonicotinamide dihydrochloride |
| X-286 | 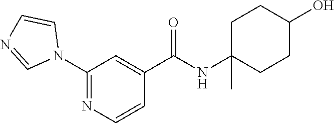 [SMILES: CC1(NC(=O)c2ccnc(-n3ccnc3)c2)CCC(O)CC1] | N-(4-hydroxy-1- methylcyclohexyl)-2-(1H-imidazol- 1-yl)isonicotinamide |
| X-287 | 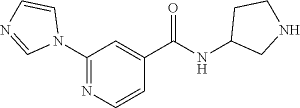 [SMILES: O=C(NC1CCNC1)c1ccnc(-n2ccnc2)c1] | 2-(1H-imidazol-1-yl)-N- (pyrrolidin-3-yl)isonicotinamide |
| X-288 | 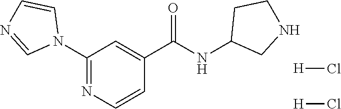 | 2-(1H-imidazol-1-yl)-N- (pyrrolidin-3-yl)isonicotinamide dihydrochloride |
| X-289 | 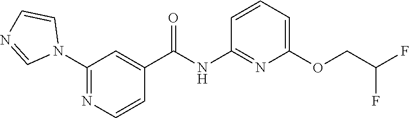 [SMILES: O=C(Nc1cccc(OCC(F)F)n1)c1ccnc(-n2ccnc2)c1] | N-(6-(2,2-difluoroethoxy)pyridin-2- yl)-2-(1H-imidazol-1- yl)isonicotinamide |
| X-290 | 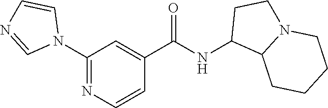 [SMILES: O=C(NC1CCN2CCCCC12)c1ccnc(-n2ccnc2)c1] | 2-(1H-imidazol-1-yl)-1- (octahydroindolizin-1- yl)isonicotinamide |
| X-291 | 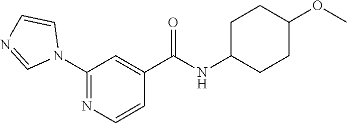 [SMILES: COC1CCC(NC(=O)c2ccnc(-n3ccnc3)c2)CC1] | 2-(1H-imidazol-1-yl)-N-(4- methoxycyclohexyl)isonicotinamide |
| X-292 | 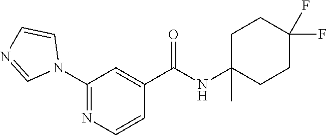 [SMILES: CC1(NC(=O)c2ccnc(-n3ccnc3)c2)CCC(F)(F)CC1] | N-(4,4-difluoro-1- methylcyclohexyl)-2-(1H-imidazol- 1-yl)isonicotinamide |
| X-293 | 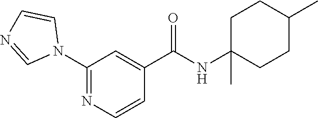 [SMILES: CC1CCC(C)(NC(=O)c2ccnc(-n3ccnc3)c2)CC1] | N-(1,4-dimethylcyclohexyl)-2-(1H- imidazol-1-yl)isonicotinamide |
| X-294 | 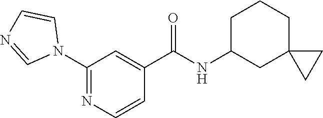 [SMILES: O=C(NC1CCCC2(CC2)C1)c1ccnc(-n2ccnc2)c1] | 2-(1H-imidazol-1-yl)-N- (spiro[2.5]octan-5- yl)isonicotinamide |
| X-295 | 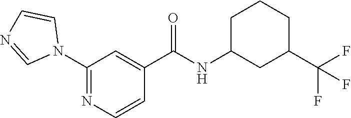 [SMILES: O=C(NC1CCCC(C(F)(F)F)C1)c1ccnc(-n2ccnc2)c1] | 2-(1H-imidazol-1-yl)-N-(3- (trifluoromethyl)cyclohexyl) isonicotinamide |
| X-296 | 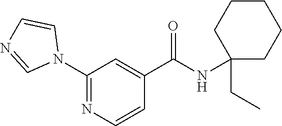 [SMILES: CCC1(NC(=O)c2ccnc(-n3ccnc3)c2)CCCCC1] | N-(1-ethylcyclohexyl)-2-(1H- imidazol-1-yl)isonicotinamide |
| X-297 | 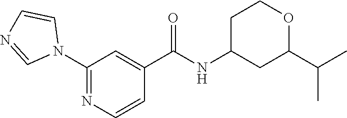 [SMILES: CC(C)C1CC(NC(=O)c2ccnc(-n3ccnc3)c2)CCO1] | 2-(1H-imidazol-1-yl)-N-(2- isopropyltetrahydro-2H-pyran-4- yl)isonicotinamide |
| X-298 | 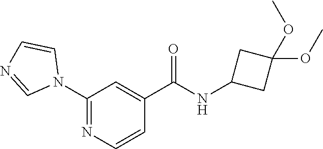 [SMILES: COC1(OC)CC(NC(=O)c2ccnc(-n3ccnc3)c2)C1] | N-(3,3-dimethoxycyclobutyl)-2- (1H-imidazol-1-yl)isonicotinamide |
| X-299 | 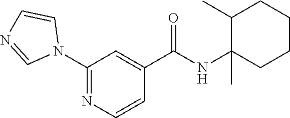 [SMILES: CC1CCCCC1(C)NC(=O)c1ccnc(-n2ccnc2)c1] | N-(1,2-dimethylcyclohexyl)-2-(1H- imidazol-1-yl)isonicotinamide |
| X-300 | 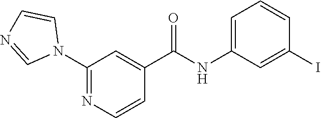 [SMILES: O=C(Nc1cccc(I)c1)c1ccnc(-n2ccnc2)c1] | 2-(1H-imidazol-1-yl)-N-(3- iodophenyl)isonicotinamide |
| X-301 | 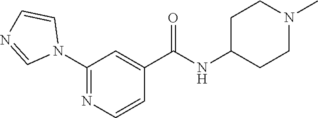 [SMILES: CN1CCC(NC(=O)c2ccnc(-n3ccnc3)c2)CC1] | 2-(1H-imidazol-1-yl)-N-(1- methylpiperidin-4- yl)isonicotinamide |
| X-302 | 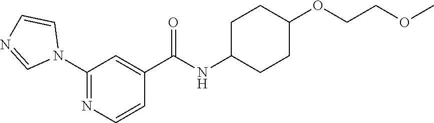 [SMILES: COCCOC1CCC(NC(=O)c2ccnc(-n3ccnc3)c2)CC1] | 2-(1H-imidazol-1-yl)-N-(4-(2- methoxyethoxy)cyclohexyl) isonicotinamide |
| X-303 | 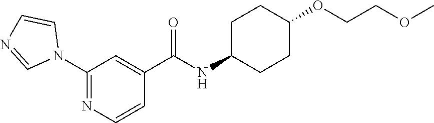 [SMILES: COCCO[C@H]1CC[C@H](NC(=O)c2ccnc(-n3ccnc3)c2)CC1] | 2-(1H-imidazol-1-yl)-N-((1r,4r)-4- (2-methoxyethoxy)cyclohexyl) isonicotinamide |
| X-304 | 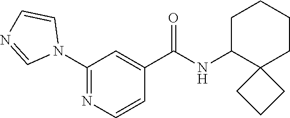 [SMILES: O=C(NC1CCCCC12CCC2)c1ccnc(-n2ccnc2)c1] | 2-(1H-imidazol-1-yl)-N- (spiro[3.5]nonan-5- yl)isonicotinamide |
| X-305 | 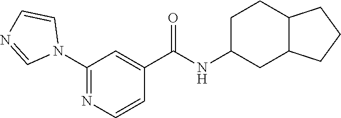 [SMILES: O=C(NC1CCC2CCCC2C1)c1ccnc(-n2ccnc2)c1] | 2-(1H-imidazol-1-yl)-N- (octahydro-1H-inden-5- yl)isonicotinamide |
| X-306 | 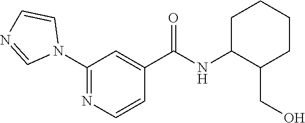 [SMILES: O=C(NC1CCCCC1CO)c1ccnc(-n2ccnc2)c1] | N-(2-(hydroxymethyl)cyclohexyl)- 2-(1H-imidazol-1- yl)isonicotinamide |
| X-307 | 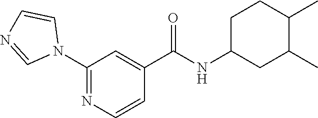 [SMILES: CC1CCC(NC(=O)c2ccnc(-n3ccnc3)c2)CC1C] | N-(3,4-dimethylcyclohexyl)-2-(1H- imidazol-1-yl)isonicotinamide |
| X-308 | 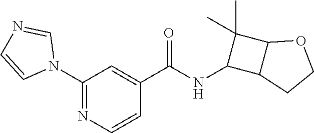 [SMILES: CC1(C)C(NC(=O)c2ccnc(-n3ccnc3)c2)C2CCOC21] | N-(7,7-dimethyl-2- oxabicyclo[3.2.0]heptan-6-yl)-2- (1H-imidazol-1-yl)isonicotinamide |
| X-309 | 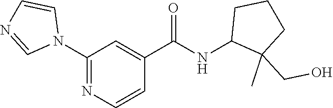 [SMILES: CC1(CO)CCCC1NC(=O)c1ccnc(-n2ccnc2)c1] | N-(2-(hydroxymethyl)-2- methylcyclopentyl)-2-(1H- imidazol-1-yl)isonicotinamide |
| X-310 | 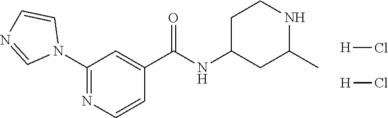 | 2-(1H-imidazol-1-yl)-N-(2- methylpiperidin-4- yl)isonicotinamide dihydrochloride |
| X-311 | 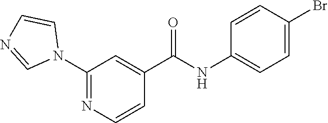 [SMILES: O=C(Nc1ccc(Br)cc1)c1ccnc(-n2ccnc2)c1] | N-(4-bromophenyl)-2-(1H- imidazol-1-yl)isonicotinamide |
| X-312 | 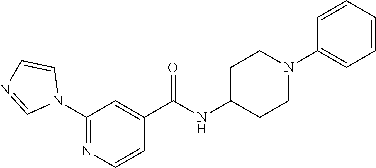 [SMILES: O=C(NC1CCN(c2ccccc2)CC1)c1ccnc(-n2ccnc2)c1] | 2-(1H-imidazol-1-yl)-N-(1- phenylpiperidin-4- yl)isonicotinamide |
| X-313 | 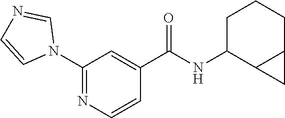 [SMILES: O=C(NC1CCCC2CC21)c1ccnc(-n2ccnc2)c1] | N-(bicyclo[4.1.0]heptan-2-yl)-2- (1H-imidazol-1-yl)isonicotinamide |
| X-314 | 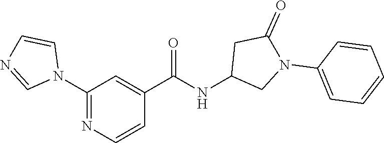 [SMILES: O=C(NC1CC(=O)N(c2ccccc2)C1)c1ccnc(-n2ccnc2)c1] | 2-(1H-imidazol-1-yl)-N-(5-oxo-1- phenylpyrrolidin-3- yl)isonicotinamide |
| X-315 | 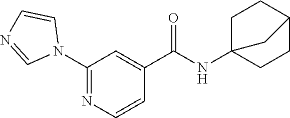 [SMILES: O=C(NC12CCC(CC1)C2)c1ccnc(-n2ccnc2)c1] | N-(bicyclo[2.2.1]heptan-1-yl)-2- (1H-imidazol-1-yl)isonicotinamide |
| X-316 | 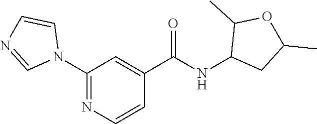 [SMILES: CC1CC(NC(=O)c2ccnc(-n3ccnc3)c2)C(C)O1] | N-(2,5-dimethyltetrahydrofuran-3- yl)-2-(1H-imidazol-1- yl)isonicotinamide |
| X-317 | 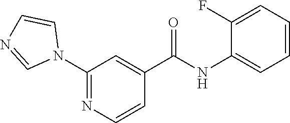 [SMILES: O=C(Nc1ccccc1F)c1ccnc(-n2ccnc2)c1] | N-(2-fluorophenyl)-2-(1H- imidazol-1-yl)isonicotinamide |
| X-318 | 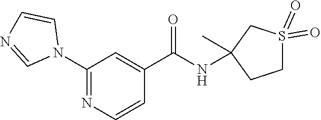 [SMILES: CC1(NC(=O)c2ccnc(-n3ccnc3)c2)CCS(=O)(=O)C1] | 2-(1H-imidazol-1-yl)-N-(3-methyl- 1,1-dioxidotetrahydrothiophen-3- yl)isonicotinamide |
| X-319 | 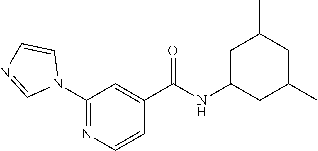 [SMILES: CC1CC(C)CC(NC(=O)c2ccnc(-n3ccnc3)c2)C1] | N-(3,5-dimethylcyclohexyl)-2-(1H- imidazol-1-yl)isonicotinamide |
| X-320 | 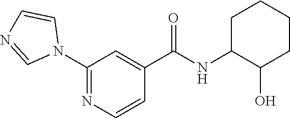 [SMILES: O=C(NC1CCCCC1O)c1ccnc(-n2ccnc2)c1] | N-(2-hydroxycyclohexyl)-2-(1H- imidazol-1-yl)isonicotinamide |
| X-321 | 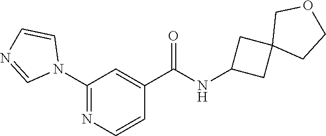 [SMILES: O=C(NC1CC2(CCOC2)C1)c1ccnc(-n2ccnc2)c1] | 2-(1H-imidazol-1-yl)-N-(6- oxaspiro[3.4]octan-2- yl)isonicotinamide |
| X-322 | 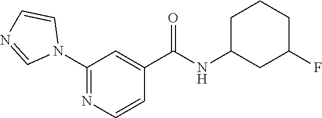 [SMILES: O=C(NC1CCCC(F)C1)c1ccnc(-n2ccnc2)c1] | N-(3-fluorocyclohexyl)-2-(1H- imidazol-1-yl)isonicotinamide |
| X-323 | 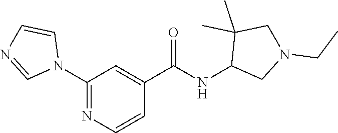 [SMILES: CCN1CC(NC(=O)c2ccnc(-n3ccnc3)c2)C(C)(C)C1] | N-(1-ethyl-4,4-dimethylpyrrolidin- 3-yl)-2-(1H-imidazol-1- yl)isonicotinamide |
| X-324 | 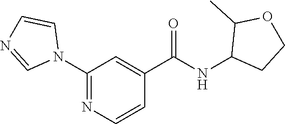 [SMILES: CC1OCCC1NC(=O)c1ccnc(-n2ccnc2)c1] | 2-(1H-imidazol-1-yl)-N-(2- methyltetrahydrofuran-3- yl)isonicotinamide |
| X-325 | 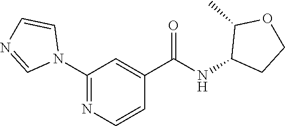 [SMILES: C[C@@H]1OCC[C@@H]1NC(=O)c1ccnc(-n2ccnc2)c1] | 2-(1H-imidazol-1-yl)-N-((2S,3S)-2- methyltetrahydrofuran-3- yl)isonicotinamide |
| X-326 | 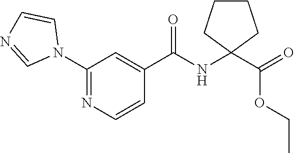 [SMILES: CCOC(=O)C1(NC(=O)c2ccnc(-n3ccnc3)c2)CCCC1] | ethyl 1-(2-(1H-imidazol-1- yl)isonicotinamido)cyclopentane-1- carboxylate |
| X-327 | 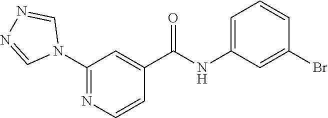 [SMILES: O=C(Nc1cccc(Br)c1)c1ccnc(-n2cnnc2)c1] | N-(3-bromophenyl)-2-(4H-1,2,4- triazol-4-yl)isonicotinamide |
| X-328 | 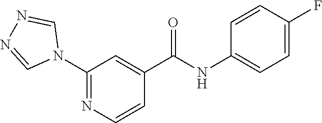 [SMILES: O=C(Nc1ccc(F)cc1)c1ccnc(-n2cnnc2)c1] | N-(4-fluorophenyl)-2-(4H-1,2,4- triazol-4-yl)isonicotinamide |
| X-329 | 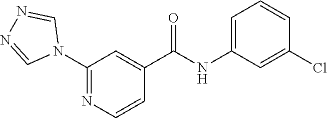 [SMILES: O=C(Nc1cccc(Cl)c1)c1ccnc(-n2cnnc2)c1] | N-(3-chlorophenyl)-2-(4H-1,2,4- triazol-4-yl)isonicotinamide |
| X-330 | 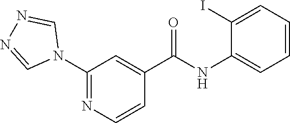 [SMILES: O=C(Nc1ccccc1I)c1ccnc(-n2cnnc2)c1] | N-(2-iodophenyl)-2-(4H-1,2,4- triazol-4-yl)isonicotinamide |
| X-331 | 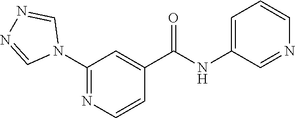 [SMILES: O=C(Nc1cccnc1)c1ccnc(-n2cnnc2)c1] | N-(pyridin-3-yl)-2-(4H-1,2,4- triazol-4-yl)isonicotinamide |
| X-332 | 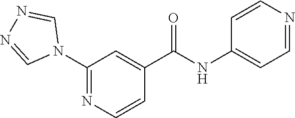 [SMILES: O=C(Nc1ccncc1)c1ccnc(-n2cnnc2)c1] | N-(pyridin-4-yl)-2-(4H-1,2,4- triazol-4-yl)isonicotinamide |
| X-333 | 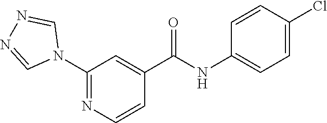 [SMILES: O=C(Nc1ccc(Cl)cc1)c1ccnc(-n2cnnc2)c1] | N-(4-chlorophenyl)-2-(4H-1,2,4- triazol-4-yl)isonicotinamide |
| X-334 | 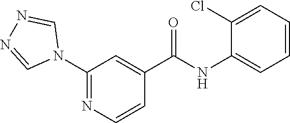 [SMILES: O=C(Nc1ccccc1Cl)c1ccnc(-n2cnnc2)c1] | N-(2-chlorophenyl)-2-(4H-1,2,4- triazol-4-yl)isonicotinamide |
| X-335 | 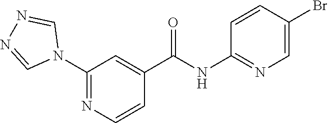 [SMILES: O=C(Nc1ccc(Br)cn1)c1ccnc(-n2cnnc2)c1] | N-(5-bromopyridin-2-yl)-2-(4H- 1,2,4-triazol-4-yl)isonicotinamide |
| X-336 | 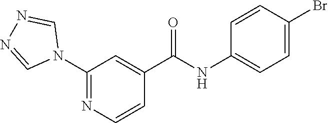 [SMILES: O=C(Nc1ccc(Br)cc1)c1ccnc(-n2cnnc2)c1] | N-(4-bromophenyl)-2-(4H-1,2,4- triazol-4-yl)isonicotinamide |
| X-337 | 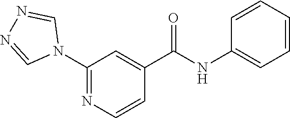 [SMILES: O=C(Nc1ccccc1)c1ccnc(-n2cnnc2)c1] | N-phenyl-2-(4H-1,2,4-triazol-4- yl)isonicotinamide |
| X-338 | 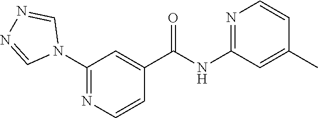 [SMILES: Cc1ccnc(NC(=O)c2ccnc(-n3cnnc3)c2)c1] | N-(4-methylpyridin-2-yl)-2-(4H- 1,2,4-triazol-4-yl)isonicotinamide |
| X-339 | 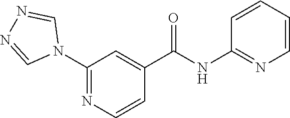 [SMILES: O=C(Nc1ccccn1)c1ccnc(-n2cnnc2)c1] | N-(pyridin-2-yl)-2-(4H-1,2,4- triazol-4-yl)isonicotinamide |
| X-340 | 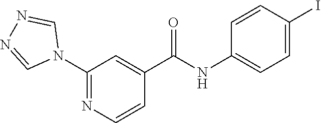 [SMILES: O=C(Nc1ccc(I)cc1)c1ccnc(-n2cnnc2)c1] | N-(4-iodophenyl)-2-(4H-1,2,4- triazol-4-yl)isonicotinamide |
| X-341 | 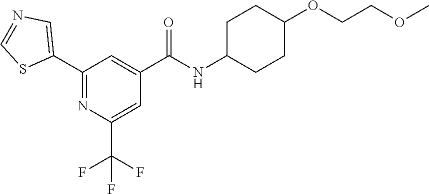 [SMILES: COCCOC1CCC(NC(=O)c2cc(-c3cncs3)nc(C(F)(F)F)c2)CC1] | methoxyethoxy)cyclohexyl)-2- (thiazol-5-yl)-6- (trifluoromethyl)isonicotinamide |
| X-342 | 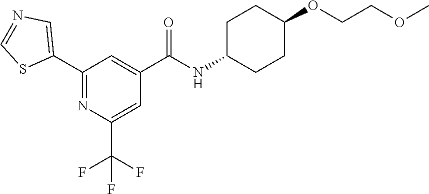 [SMILES: COCCO[C@H]1CC[C@H](NC(=O)c2cc(-c3cncs3)nc(C(F)(F)F)c2)CC1] | N-((1r,4r)-4-(2- methoxyethoxy)cyclohexyl)-2- (thiazol-5-yl)-6- (trifluoromethyl)isonicotinamide |
| X-343 | 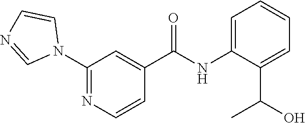 [SMILES: CC(O)c1ccccc1NC(=O)c1ccnc(-n2ccnc2)c1] | N-(2-(1-hydroxyethyl)phenyl)-2- (1H-imidazol-1-yl)isonicotinamide |
| X-344 | 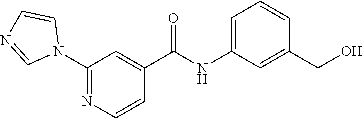 [SMILES: O=C(Nc1cccc(CO)c1)c1ccnc(-n2ccnc2)c1] | N-(3-(hydroxymethyl)phenyl)-2- (1H-imidazol-1-yl)isonicotinamide |

TABLE 4X

| Compound | | |
|---|---|---|
| Number | Structure | Name |
| X-345 | 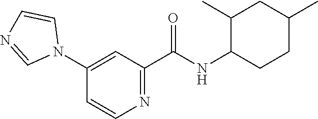 [SMILES: CC1CCC(NC(=O)c2cc(-n3ccnc3)ccn2)C(C)C1] | N-(2,4-dimethylcyclohexyl)-4-(1H- imidazol-1-yl)picolinamide |
| X-346 | 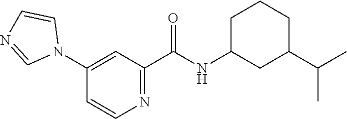 [SMILES: CC(C)C1CCCC(NC(=O)c2cc(-n3ccnc3)ccn2)C1] | 4-(1H-imidazol-1-yl)-N-(3- isopropylcyclohexyl)picolinamide |
| X-347 | 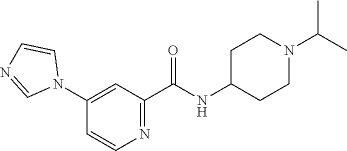 [SMILES: CC(C)N1CCC(NC(=O)c2cc(-n3ccnc3)ccn2)CC1] | 4-(1H-imidazol-1-yl)-N-(1- isopropylpiperidin-4- yl)picolinamide |
| X-348 | 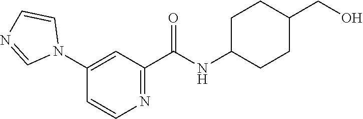 [SMILES: O=C(NC1CCC(CO)CC1)c1cc(-n2ccnc2)ccn1] | N-(4-(hydroxymethyl)cyclohexyl)- 4-(1H-imidazol-1-yl)picolinamide |
| X-349 | 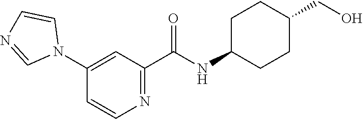 [SMILES: O=C(N[C@H]1CC[C@H](CO)CC1)c1cc(-n2ccnc2)ccn1] | N-((1r,4r)-4- (hydroxymethyl)cyclohexyl)-4- (1H-imidazol-1-yl)picolinamide |
| X-350 | 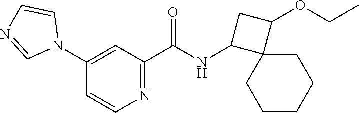 [SMILES: CCOC1CC(NC(=O)c2cc(-n3ccnc3)ccn2)C12CCCCC2] | N-(3-ethoxyspiro[3.5]nonan-1-yl)- 4-(1H-imidazol-1-yl)picolinamide |
| X-351 | 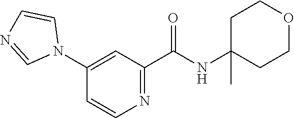 [SMILES: CC1(NC(=O)c2cc(-n3ccnc3)ccn2)CCOCC1] | 4-(1H-imidazol-1-yl)-N-(4- methyltetrahydro-2H-pyran-4- yl)picolinamide |
| X-352 | 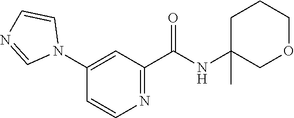 [SMILES: CC1(NC(=O)c2cc(-n3ccnc3)ccn2)CCCOC1] | 4-(1H-imidazol-1-yl)-N-(3- methyltetrahydro-2H-pyran-3- yl)picolinamide |
| X-353 | 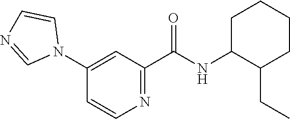 [SMILES: CCC1CCCCC1NC(=O)c1cc(-n2ccnc2)ccn1] | N-(2-ethylcyclohexyl)-4-(1H- imidazol-1-yl)picolinamide |
| X-354 | 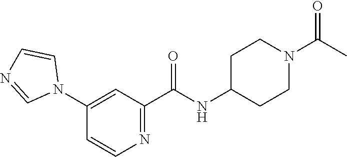 [SMILES: CC(=O)N1CCC(NC(=O)c2cc(-n3ccnc3)ccn2)CC1] | N-(1-acetylpiperidin-4-yl)-4-(1H- imidazol-1-yl)picolinamide |
| X-355 | 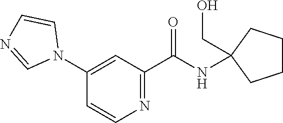 [SMILES: O=C(NC1(CO)CCCC1)c1cc(-n2ccnc2)ccn1] | N-(1-(hydroxymethyl)cyclopentyl)- 4-(1H-imidazol-1-yl)picolinamide |
| X-356 | 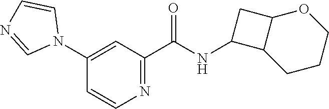 [SMILES: O=C(NC1CC2OCCCC12)c1cc(-n2ccnc2)ccn1] | N-(2-oxabicyclo[4.2.0]octan-7-yl)- 4-(1H-imidazol-1-yl)picolinamide |
| X-357 | 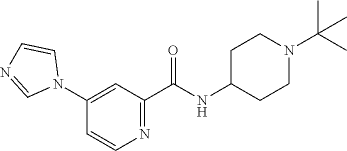 [SMILES: CC(C)(C)N1CCC(NC(=O)c2cc(-n3ccnc3)ccn2)CC1] | N-(1-(tert-butyl)piperidin-4-yl)-4- (1H-imidazol-1-yl)picolinamide |
| X-358 | 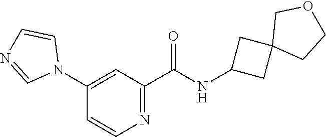 [SMILES: O=C(NC1CC2(CCOC2)C1)c1cc(-n2ccnc2)ccn1] | 4-(1H-imidazol-1-yl)-N-(6- oxaspiro[3.4]octan-2- yl)picolinamide |
| X-359 | 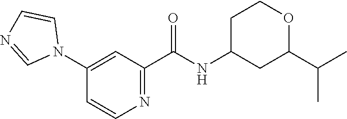 [SMILES: CC(C)C1CC(NC(=O)c2cc(-n3ccnc3)ccn2)CCO1] | 4-(1H-imidazol-1-yl)-N-(2- isopropyltetrahydro-2H-pyran-4- yl)picolinamide |
| X-360 | 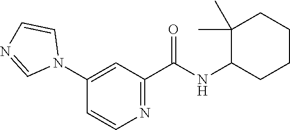 [SMILES: CC1(C)CCCCC1NC(=O)c1cc(-n2ccnc2)ccn1] | N-(2,2-dimethylcyclohexyl)-4-(1H- imidazol-1-yl)picolinamide |
| X-361 | 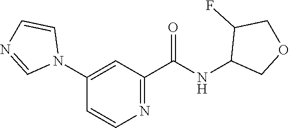 [SMILES: O=C(NC1COCC1F)c1cc(-n2ccnc2)ccn1] | N-(4-fluorotetrahydrofuran-3-yl)-4- (1H-imidazol-1-yl)picolinamide |
| X-362 | 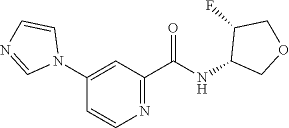 [SMILES: O=C(N[C@H]1COC[C@H]1F)c1cc(-n2ccnc2)ccn1] | N-((3S,4S)-4- fluorotetrahydrofuran-3-yl)-4-(1H- imidazol-1-yl)picolinamide |
| X-363 | 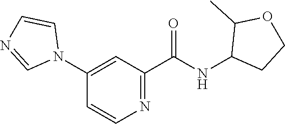 [SMILES: CC1OCCC1NC(=O)c1cc(-n2ccnc2)ccn1] | 4-(1H-imidazol-1-yl)-N-(2- methyltetrahydrofuran-3- yl)picolinamide |
| X-364 | 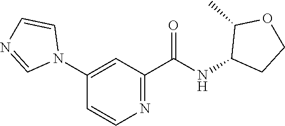 [SMILES: C[C@@H]1OCC[C@@H]1NC(=O)c1cc(-n2ccnc2)ccn1] | 4-(1H-imidazol-1-yl)-N-((2S,3S)-2- methyltetrahydrofuran-3- yl)picolinamide |
| X-365 | 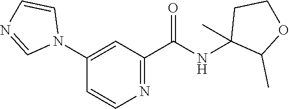 [SMILES: CC1OCCC1(C)NC(=O)c1cc(-n2ccnc2)ccn1] | N-(2,3-dimethyltetrahydrofuran-3- yl)-4-(1H-imidazol-1- yl)picolinamide |
| X-366 | 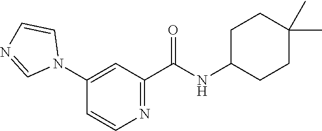 [SMILES: CC1(C)CCC(NC(=O)c2cc(-n3ccnc3)ccn2)CC1] | N-(4,4-dimethylcyclohexyl)-4-(1H- imidazol-1-yl)picolinamide |
| X-367 | 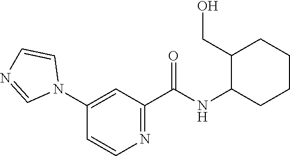 [SMILES: O=C(NC1CCCCC1CO)c1cc(-n2ccnc2)ccn1] | N-(2-(hydroxymethyl)cyclohexyl)- 4-(1H-imidazol-1-yl)picolinamide |
| X-368 | 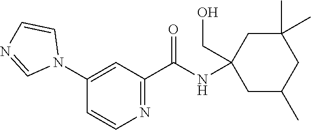 [SMILES: CC1CC(C)(C)CC(CO)(NC(=O)c2cc(-n3ccnc3)ccn2)C1] | N-(1-(hydroxymethyl)-3,3,5- trimethylcyclohexyl)-4-(1H- imidazol-1-yl)picolinamide |
| X-369 | 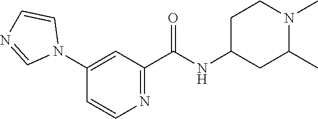 [SMILES: CC1CC(NC(=O)c2cc(-n3ccnc3)ccn2)CCN1C] | N-(1,2-dimethylpiperidin-4-yl)-4- (1H-imidazol-1-yl)picolinamide |
| X-370 | 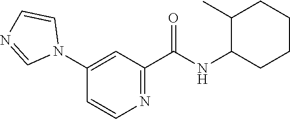 [SMILES: CC1CCCCC1NC(=O)c1cc(-n2ccnc2)ccn1] | 4-(1H-imidazol-1-yl)-N-(2- methylcyclohexyl)picolinamide |
| X-371 | 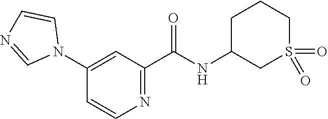 [SMILES: O=C(NC1CCCS(=O)(=O)C1)c1cc(-n2ccnc2)ccn1] | N-(1,1-dioxidotetrahydro-2H- thiopyran-3-yl)-4-(1H-imidazol-1- yl)picolinamide |
| X-372 | 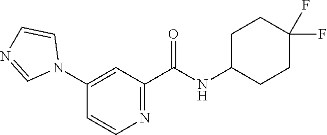 [SMILES: O=C(NC1CCC(F)(F)CC1)c1cc(-n2ccnc2)ccn1] | N-(4,4-difluorocyclohexyl)-4-(1H- imidazol-1-yl)picolinamide |
| X-373 | 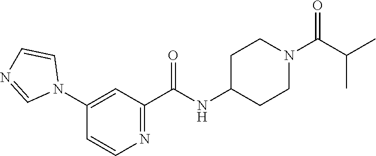 [SMILES: CC(C)C(=O)N1CCC(NC(=O)c2cc(-n3ccnc3)ccn2)CC1] | 4-(1H-imidazol-1-yl)-N-( 1- isobutyrylpiperidin-4- yl)picolinamide |
| X-374 | 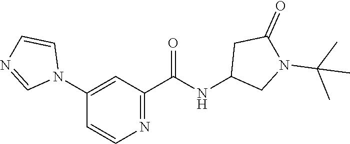 [SMILES: CC(C)(C)N1CC(NC(=O)c2cc(-n3ccnc3)ccn2)CC1=O] | N-(1-(tert-butyl)-5-oxopyrrolidin-3- yl)-4-(1H-imidazol-1- yl)picolinamide |
| X-375 | 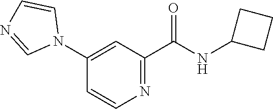 [SMILES: O=C(NC1CCC1)c1cc(-n2ccnc2)ccn1] | N-cyclobutyl-4-(1H-imidazol-1- yl)picolinamide |
| X-376 | 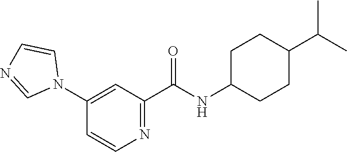 [SMILES: CC(C)C1CCC(NC(=O)c2cc(-n3ccnc3)ccn2)CC1] | 4-(1H-imidazol-1-yl)-N-(4- isopropylcyclohexyl)picolinamide |
| X-377 | 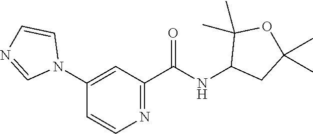 [SMILES: CC1(C)CC(NC(=O)c2cc(-n3ccnc3)ccn2)C(C)(C)O1] | 4-(1H-imidazol-1-yl)-N-(2,2,5,5- tetramethyltetrahydrofuran-3- yl)picolinamide |
| X-378 | 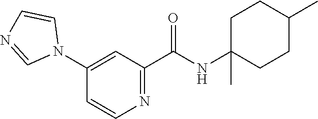 [SMILES: CC1CCC(C)(NC(=O)c2cc(-n3ccnc3)ccn2)CC1] | N-(1,4-dimethylcyclohexyl)-4-(1H- imidazol-1-yl)picolinamide |
| X-379 | 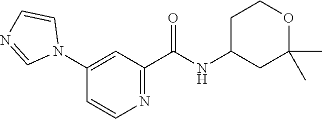 [SMILES: CC1(C)CC(NC(=O)c2cc(-n3ccnc3)ccn2)CCO1] | N-(2,2-dimethyltetrahydro-2H- pyran-4-yl)-4-(1H-imidazol-1- yl)picolinamide |
| X-380 | 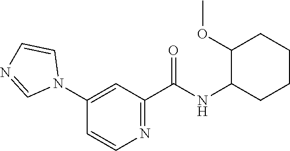 [SMILES: COC1CCCCC1NC(=O)c1cc(-n2ccnc2)ccn1] | 4-(1H-imidazol-1-yl)-N-(2- methoxycyclohexyl)picolinamide |
| X-381 | 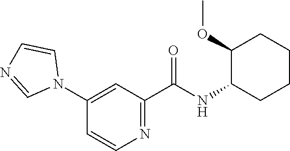 [SMILES: CO[C@H]1CCCC[C@@H]1NC(=O)c1cc(-n2ccnc2)ccn1] | 4-(1H-imidazol-1-yl)-N-((1S,2S)-2- methoxycyclohexyl)picolinamide |
| X-382 | 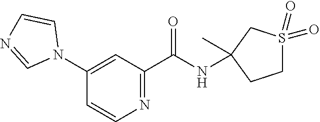 [SMILES: CC1(NC(=O)c2cc(-n3ccnc3)ccn2)CCS(=O)(=O)C1] | 4-(1H-imidazol-1-yl)-N-(3-methyl- 1,1-dioxidotetrahydrothiophen-3- yl)picolinamide |
| X-383 | 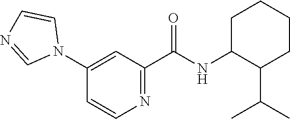 [SMILES: CC(C)C1CCCCC1NC(=O)c1cc(-n2ccnc2)ccn1] | 4-(1H-imidazol-1-yl)-N-(2- isopropylcyclohexyl)picolinamide |
| X-384 | 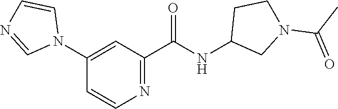 [SMILES: CC(=O)N1CCC(NC(=O)c2cc(-n3ccnc3)ccn2)C1] | N-(1-acetylpyrrolidin-3-yl)-4-(1H- imidazol-1-yl)picolinamide |
| X-385 | 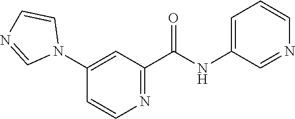 [SMILES: O=C(Nc1cccnc1)c1cc(-n2ccnc2)ccn1] | 4-(1H-imidazol-1-yl)-N-(pyridin-3- yl)picolinamide |
| X-386 | 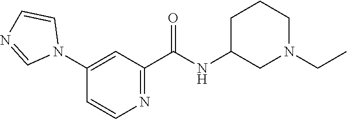 [SMILES: CCN1CCCC(NC(=O)c2cc(-n3ccnc3)ccn2)C1] | N-(1-ethylpiperidin-3-yl)-4-(1H- imidazol-1-yl)picolinamide |
| X-387 | 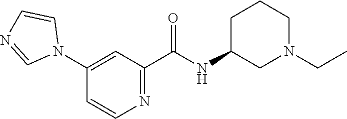 [SMILES: CCN1CCC[C@H](NC(=O)c2cc(-n3ccnc3)ccn2)C1] | (S)-N-(1-ethylpiperidin-3-yl)-4- (1H-imidazol-1-yl)picolinamide |
| X-388 | 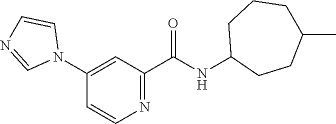 [SMILES: CC1CCCC(NC(=O)c2cc(-n3ccnc3)ccn2)CC1] | 4-(1H-imidazol-1-yl)-N-(4- methylcycloheptyl)picolinamide |
| X-389 | 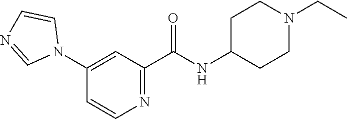 [SMILES: CCN1CCC(NC(=O)c2cc(-n3ccnc3)ccn2)CC1] | N-(1-ethylpiperidin-4-yl)-4-(1H- imidazol-1-yl)picolinamide |
| X-390 | 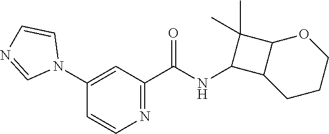 [SMILES: CC1(C)C(NC(=O)c2cc(-n3ccnc3)ccn2)C2CCCOC21] | N-(8,8-dimethyl-2- oxabicyclo[4.2.0]octan-7-yl)-4- (1H-imidazol-1-yl)picolinamide |
| X-391 | 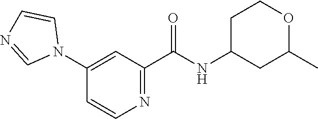 [SMILES: CC1CC(NC(=O)c2cc(-n3ccnc3)ccn2)CCO1] | 4-(1H-imidazol-1-yl)-N-(2- methyltetrahydro-2H-pyran-4- yl)picolinamide |
| X-392 | 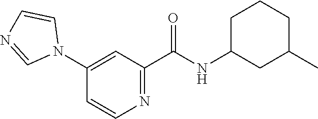 [SMILES: CC1CCCC(NC(=O)c2cc(-n3ccnc3)ccn2)C1] | 4-(1H-imidazol-1-yl)-N-(3- methylcyclohexyl)picolinamide |
| X-393 | 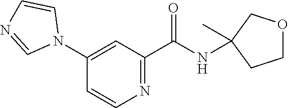 [SMILES: CC1(NC(=O)c2cc(-n3ccnc3)ccn2)CCOC1] | 4-(1H-imidazol-1-yl)-N-(3- methyltetrahydrofuran-3- yl)picolinamide |
| X-394 | 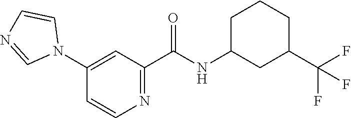 [SMILES: O=C(NC1CCCC(C(F)(F)F)C1)c1cc(-n2ccnc2)ccn1] | 4-(1H-imidazol-1-yl)-N-(3- (trifluoromethyl)cyclohexyl) picolinamide |
| X-395 | 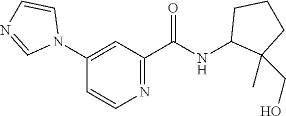 [SMILES: CC1(CO)CCCC1NC(=O)c1cc(-n2ccnc2)ccn1] | N-(2-(hydroxymethyl)-2- methylcyclopentyl)-4-(1H- imidazol-1-yl)picolinamide |
| X-396 | 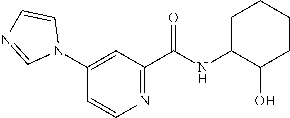 [SMILES: O=C(NC1CCCCC1O)c1cc(-n2ccnc2)ccn1] | N-(2-hydroxycyclohexyl)-4-(1H- imidazol-1-yl)picolinamide |
| X-397 | 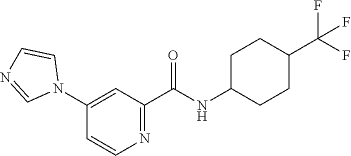 [SMILES: O=C(NC1CCC(C(F)(F)F)CC1)c1cc(-n2ccnc2)ccn1] | 4-(1H-imidazol-1-yl)-N-(4- (trifluoromethyl)cyclohexyl) picolinamide |
| X-398 | 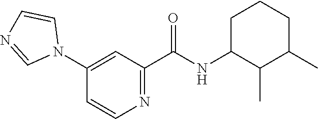 [SMILES: CC1CCCC(NC(=O)c2cc(-n3ccnc3)ccn2)C1C] | N-(2,3-dimethylcyclohexyl)-4-(1H- imidazol-1-yl)picolinamide |
| X-399 | 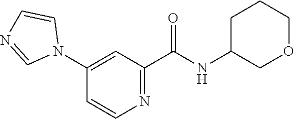 [SMILES: O=C(NC1CCCOC1)c1cc(-n2ccnc2)ccn1] | 4-(1H-imidazol-1-yl)-N- (tetrahydro-2H-pyran-3- yl)picolinamide |
| X-400 | 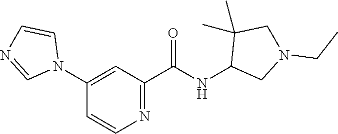 [SMILES: CCN1CC(NC(=O)c2cc(-n3ccnc3)ccn2)C(C)(C)C1] | N-(1-ethyl-4,4-dimethylpyrrolidin- 3-yl)-4-(1H-imidazol-1- yl)picolinamide |
| X-401 | 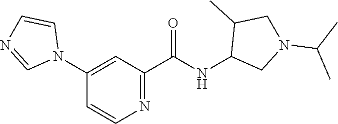 [SMILES: CC1CN(C(C)C)CC1NC(=O)c1cc(-n2ccnc2)ccn1] | 4-(1H-imidazol-1-yl)-N-(1- isopropyl-4-methylpyrrolidin-3- yl)picolinamide |
| X-402 | 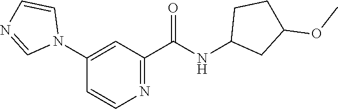 [SMILES: COC1CCC(NC(=O)c2cc(-n3ccnc3)ccn2)C1] | 4-(1H-imidazol-1-yl)-N-(3- methoxycyclopentyl)picolinamide |
| X-403 | 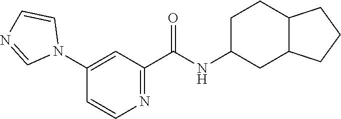 [SMILES: O=C(NC1CCC2CCCC2C1)c1cc(-n2ccnc2)ccn1] | 4-(1H-imidazol-1-yl)-N- (octahydro-1H-inden-5- yl)picolinamide |
| X-404 | 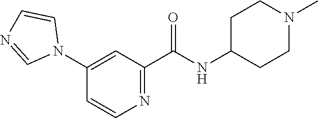 [SMILES: CN1CCC(NC(=O)c2cc(-n3ccnc3)ccn2)CC1] | 4-(1H-imidazol-1-yl)-N-(1- methylpiperidin-4-yl)picolinamide |
| X-405 | 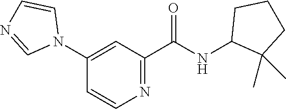 [SMILES: CC1(C)CCCC1NC(=O)c1cc(-n2ccnc2)ccn1] | N-(2,2-dimethylcyclopentyl)-4- (1H-imidazol-1-yl)picolinamide |
| X-406 | 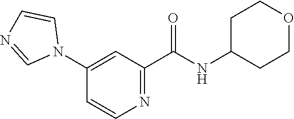 [SMILES: O=C(NC1CCOCC1)c1cc(-n2ccnc2)ccn1] | 4-(1H-imidazol-1-yl)-N- (tetrahydro-2H-pyran-4- yl)picolinamide |
| X-407 | 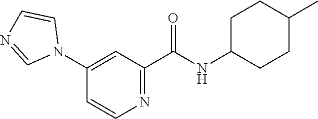 [SMILES: CC1CCC(NC(=O)c2cc(-n3ccnc3)ccn2)CC1] | 4-(1H-imidazol-1-yl)-N-(4- methylcyclohexyl)picolinamide |
| X-408 | 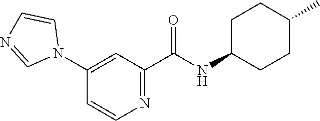 [SMILES: C[C@H]1CC[C@H](NC(=O)c2cc(-n3ccnc3)ccn2)CC1] | 4-(1H-imidazol-1-yl)-N-((1r,4r)-4- methylcyclohexyl)picolinamide |
| X-409 | 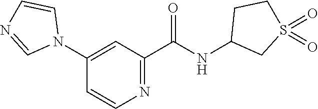 [SMILES: O=C(NC1CCS(=O)(=O)C1)c1cc(-n2ccnc2)ccn1] | N-(1,1-dioxidotetrahydrothiophen- 3-yl)-4-(1H-imidazol-1- yl)picolinamide |
| X-410 | 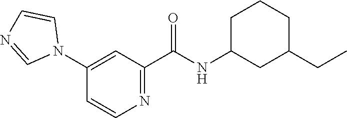 [SMILES: CCC1CCCC(NC(=O)c2cc(-n3ccnc3)ccn2)C1] | N-(3-ethylcyclohexyl)-4-(1H- imidazol-1-yl)picolinamide |
| X-411 | 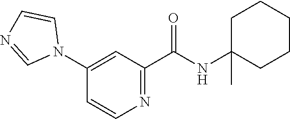 [SMILES: CC1(NC(=O)c2cc(-n3ccnc3)ccn2)CCCCC1] | 4-(1H-imidazol-1-yl)-N-(1- methylcyclohexyl)picolinamide |
| X-412 | 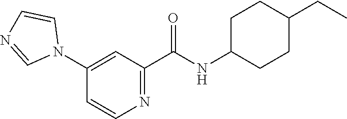 [SMILES: CCC1CCC(NC(=O)c2cc(-n3ccnc3)ccn2)CC1] | N-(4-ethylcyclohexyl)-4-(1H- imidazol-1-yl)picolinamide |
| X-413 | 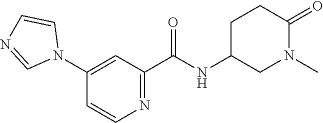 [SMILES: CN1CC(NC(=O)c2cc(-n3ccnc3)ccn2)CCC1=O] | 4-(1H-imidazol-1-yl)-N-(1-methyl- 6-oxopiperidin-3-yl)picolinamide |
| X-414 | 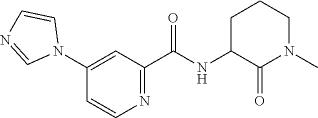 [SMILES: CN1CCCC(NC(=O)c2cc(-n3ccnc3)ccn2)C1=O] | 4-(1H-imidazol-1-yl)-N-(1-methyl- 2-oxopiperidin-3-yl)picolinamide |
| X-415 | 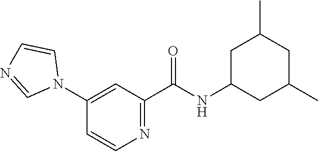 [SMILES: CC1CC(C)CC(NC(=O)c2cc(-n3ccnc3)ccn2)C1] | N-(3,5-dimethylcyclohexyl)-4-(1H- imidazol-1-yl)picolinamide |

TABLE 5X

| Compound | | |
|---|---|---|
| Number | Structure | Name |
| X-416 | 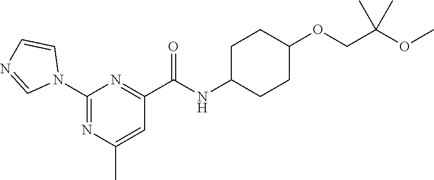 [SMILES: COC(C)(C)COC1CCC(NC(=O)c2cc(C)nc(-n3ccnc3)n2)CC1] | 2-(1H-imidazol-1-yl)-N-(4-(2- methoxy-2- methylpropoxy)cyclohexyl)-6- methylpyrimidine-4-carboxamide |
| X-417 | 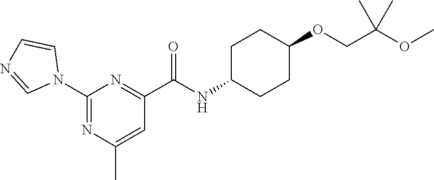 [SMILES: COC(C)(C)CO[C@H]1CC[C@H](NC(=O)c2cc(C)nc(-n3ccnc3)n2)CC1] | 2-(1H-imidazol-1-yl)-N-((1r,4r)-4- (2-methoxy-2- methylpropoxy)cyclohexyl)-6- methylpyrimidine-4-carboxamide |
| X-418 | 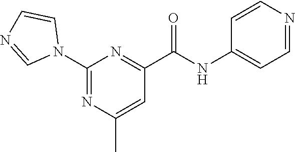 [SMILES: Cc1cc(C(=O)Nc2ccncc2)nc(-n2ccnc2)n1] | 2-(1H-imidazol-1-yl)-6-methyl-N- (pyridin-4-yl)pyrimidine-4- carboxamide |
| X-419 | 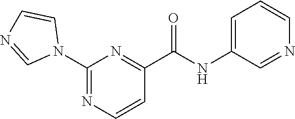 [SMILES: O=C(Nc1cccnc1)c1ccnc(-n2ccnc2)n1] | 2-(1H-imidazol-1-yl)-N-(pyridin-3- yl)pyrimidine-4-carboxamide |
| X-420 | 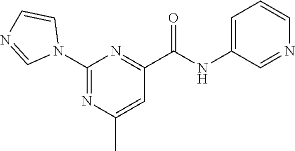 [SMILES: Cc1cc(C(=O)Nc2cccnc2)nc(-n2ccnc2)n1] | 2-(1H-imidazol-1-yl)-6-methyl-N- (pyridin-3-yl)pyrimidine-4- carboxamide |
| X-421 | 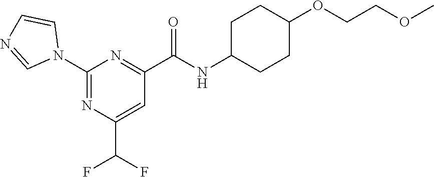 [SMILES: COCCOC1CCC(NC(=O)c2cc(C(F)F)nc(-n3ccnc3)n2)CC1] | 6-(difluoromethyl)-2-(1H-imidazol- 1-yl)-N-(4-(2- methoxyethoxy)cyclohexyl) pyrimidine-4-carboxamide |
| X-422 | 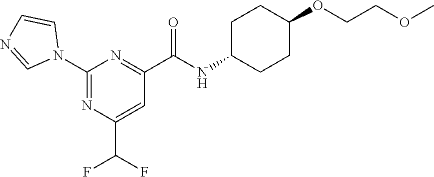 [SMILES: COCCO[C@H]1CC[C@H](NC(=O)c2cc(C(F)F)nc(-n3ccnc3)n2)CC1] | 6-(difluoromethyl)-2-(1H-imidazol- 1-yl)-N-((1r,4r)-4-(2- methoxyethoxy)cyclohexyl) pyrimidine-4-carboxamide |
| X-423 | 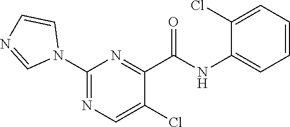 [SMILES: O=C(Nc1ccccc1Cl)c1nc(-n2ccnc2)ncc1Cl] | 5-chloro-N-(2-chlorophenyl)-2- (1H-imidazol-1-yl)pyrimidine-4- carboxamide |
| X-424 | 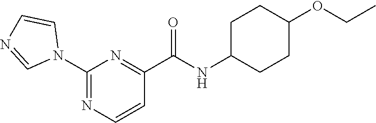 [SMILES: CCOC1CCC(NC(=O)c2ccnc(-n3ccnc3)n2)CC1] | N-(4-ethoxycyclohexyl)-2-(1H- imidazol-1-yl)pyrimidine-4- carboxamide |
| X-425 | 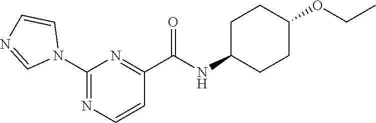 [SMILES: CCO[C@H]1CC[C@H](NC(=O)c2ccnc(-n3ccnc3)n2)CC1] | N-((1r,4r)-4-ethoxycyclohexyl)-2- (1H-imidazol-1-yl)pyrimidine-4- carboxamide |
| X-426 | 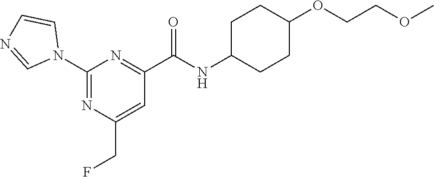 [SMILES: COCCOC1CCC(NC(=O)c2cc(CF)nc(-n3ccnc3)n2)CC1] | 6-(fluoromethyl)-2-(1H-imidazol-1- yl)-N-(4-(2- methoxyethoxy)cyclohexyl) pyrimidine-4-carboxamide |
| X-427 | 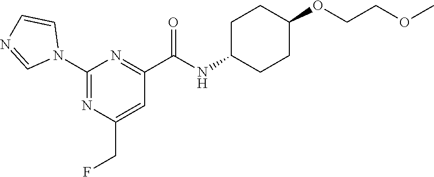 [SMILES: COCCO[C@H]1CC[C@H](NC(=O)c2cc(CF)nc(-n3ccnc3)n2)CC1] | 6-(fluoromethyl)-2-(1H-imidazol-1- yl)-N-((1r,4r)-4-(2- methoxyethoxy)cyclohexyl) pyrimidine-4-carboxamide |
| X-428 | 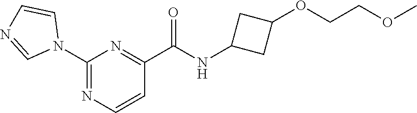 [SMILES: COCCOC1CC(NC(=O)c2ccnc(-n3ccnc3)n2)C1] | 2-(1H-imidazol-1-yl)-N-(3-(2- methoxyethoxy)cyclobutyl) pyrimidine-4-carboxamide |
| X-429 | 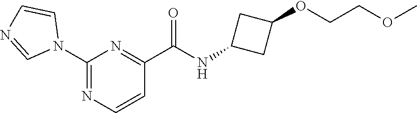 [SMILES: COCCO[C@H]1C[C@H](NC(=O)c2ccnc(-n3ccnc3)n2)C1] | 2-(1H-imidazol-1-yl)-N-((1r,3r)-3- (2- methoxyethoxy)cyclobutyl) pyrimidine-4-carboxamide |
| X-430 | 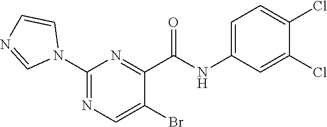 [SMILES: O=C(Nc1ccc(Cl)c(Cl)c1)c1nc(-n2ccnc2)ncc1Br] | 5-bromo-N-(3,4-dichlorophenyl)-2- (1H-imidazol-1-yl)pyrimidine-4- carboxamide |
| X-431 | 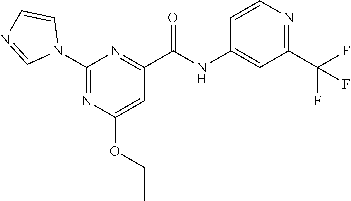 [SMILES: CCOc1cc(C(=O)Nc2ccnc(C(F)(F)F)c2)nc(-n2ccnc2)n1] | 6-ethoxy-2-(1H-imidazol-1-yl)-N- (2-(trifluoromethyl)pyridin-4- yl)pyrimidine-4-carboxamide |
| X-432 | 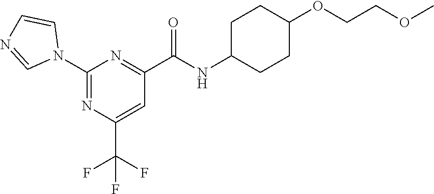 [SMILES: COCCOC1CCC(NC(=O)c2cc(C(F)(F)F)nc(-n3ccnc3)n2)CC1] | 2-(1H-imidazol-1-yl)-N-(4-(2- methoxyethoxy)cyclohexyl)-6- (trifluoromethyl)pyrimidine-4- carboxamide |
| X-433 | 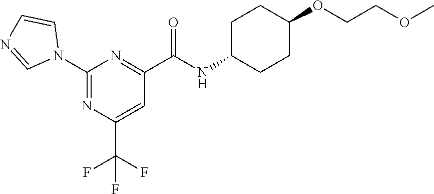 [SMILES: COCCO[C@H]1CC[C@H](NC(=O)c2cc(C(F)(F)F)nc(-n3ccnc3)n2)CC1] | 2-(1H-imidazol-1-yl)-N-((1r,4r)-4- (2-methoxyethoxy)cyclohexyl)-6- (trifluoromethyl)pyrimidine-4- carboxamide |
| X-434 | 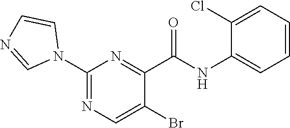 [SMILES: O=C(Nc1ccccc1Cl)c1nc(-n2ccnc2)ncc1Br] | 5-bromo-N-(2-chlorophenyl)-2- (1H-imidazol-1-yl)pyrimidine-4- carboxamide |
| X-435 | 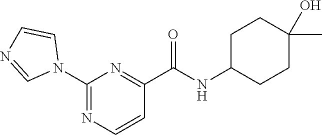 [SMILES: CC1(O)CCC(NC(=O)c2ccnc(-n3ccnc3)n2)CC1] | N-(4-hydroxy-4- methylcyclohexyl)-2-(1H-imidazol- 1-yl)pyrimidine-4-carboxamide |
| X-436 | 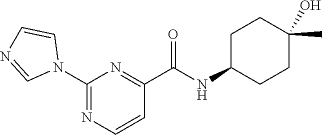 [SMILES: C[C@]1(O)CC[C@H](NC(=O)c2ccnc(-n3ccnc3)n2)CC1] | N-((1r,4r)-4-hydroxy-4- methylcyclohexyl)-2-(1H-imidazol- 1-yl)pyrimidine-4-carboxamide |
| X-437 | 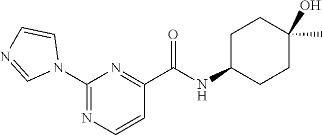 [SMILES: C[C@]1(O)CC[C@@H](NC(=O)c2ccnc(-n3ccnc3)n2)CC1] | N-((1s,4s)-4-hydroxy-4- methylcyclohexyl)-2-(1H-imidazol- 1-yl)pyrimidine-4-carboxamide |
| X-438 | 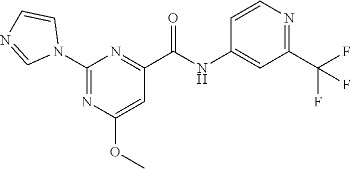 [SMILES: COc1cc(C(=O)Nc2ccnc(C(F)(F)F)c2)nc(-n2ccnc2)n1] | 2-(1H-imidazol-1-yl)-6-methoxy- N-(2-(trifluoromethyl)pyridin-4- yl)pyrimidine-4-carboxamide |
| X-439 | 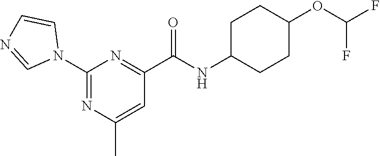 [SMILES: Cc1cc(C(=O)NC2CCC(OC(F)F)CC2)nc(-n2ccnc2)n1] | N-(4- (difluoromethoxy)cyclohexyl)-2- (1H-imidazol-1-y1)-6- methylpyrimidine-4-carboxamide |
| X-440 | 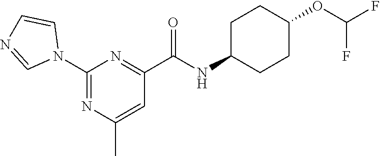 [SMILES: Cc1cc(C(=O)N[C@H]2CC[C@H](OC(F)F)CC2)nc(-n2ccnc2)n1] | N-((1r,4r)-4- (difluoromethoxy)cyclohexyl)-2- (1H-imidazol-1-yl)-6- methylpyrimidine-4-carboxamide |
| X-441 | 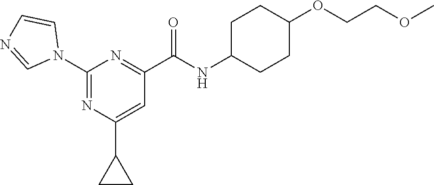 [SMILES: COCCOC1CCC(NC(=O)c2cc(C3CC3)nc(-n3ccnc3)n2)CC1] | 6-cyclopropyl-2-(1H-imidazol-1- yl)-N-(4-(2- methoxyethoxy)cyclohexyl) pyrimidine-4-carboxamide |
| X-442 | 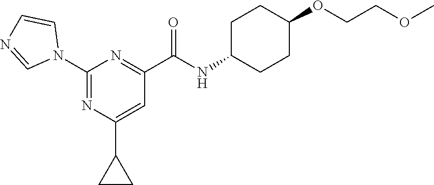 [SMILES: COCCO[C@H]1CC[C@H](NC(=O)c2cc(C3CC3)nc(-n3ccnc3)n2)CC1] | 6-cyclopropyl-2-(1H-imidazol-1- yl)-N-((1r,4r)-4-(2- methoxyethoxy)cyclohexyl) pyrimidine-4-carboxamide |
| X-443 | 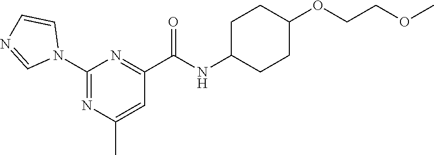 [SMILES: COCCOC1CCC(NC(=O)c2cc(C)nc(-n3ccnc3)n2)CC1] | 2-(1H-imidazol-1-yl)-N-(4-(2- methoxyethoxy)cyclohexyl)-6- methylpyrimidine-4-carboxamide |
| X-444 | 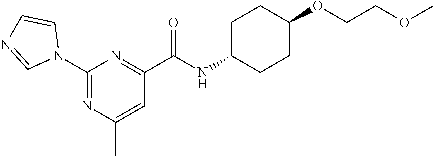 [SMILES: COCCO[C@H]1CC[C@H](NC(=O)c2cc(C)nc(-n3ccnc3)n2)CC1] | 2-(1H-imidazol-1-yl)-N-((1r,4r)-4- (2-methoxyethoxy)cyclohexyl)-6- methylpyrimidine-4-carboxamide |
| X-445 | 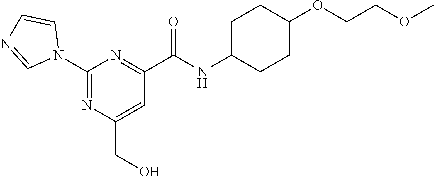 [SMILES: COCCOC1CCC(NC(=O)c2cc(CO)nc(-n3ccnc3)n2)CC1] | 6-(hydroxymethyl)-2-(1H- imidazol-1-yl)-N-(4-(2- methoxyethoxy)cyclohexyl) pyrimidine-4-carboxamide |
| X-446 | 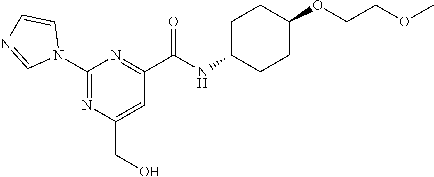 [SMILES: COCCO[C@H]1CC[C@H](NC(=O)c2cc(CO)nc(-n3ccnc3)n2)CC1] | 6-(hydroxymethyl)-2-(1H- imidazol-1-yl)-N-((1r,4r)-4-(2- methoxyethoxy)cyclohexyl) pyrimidine-4-carboxamide |
| X-447 | 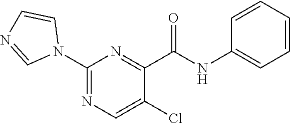 [SMILES: O=C(Nc1ccccc1)c1nc(-n2ccnc2)ncc1Cl] | 5-chloro-2-(1H-imidazol-1-yl)-N- phenylpyrimidine-4-carboxamide |
| X-448 | 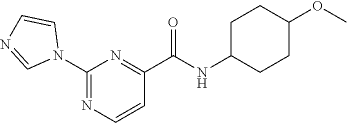 [SMILES: COC1CCC(NC(=O)c2ccnc(-n3ccnc3)n2)CC1] | 2-(1H-imidazol-1-yl)-N-(4- methoxycyclohexyl)pyrimidine-4- carboxamide |
| X-449 | 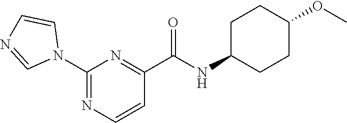 [SMILES: CO[C@H]1CC[C@H](NC(=O)c2ccnc(-n3ccnc3)n2)CC1] | 2-(1H-imidazol-1-yl)-N-((1r,4r)-4- methoxycyclohexyl)pyrimidine-4- carboxamide |
| X-450 | 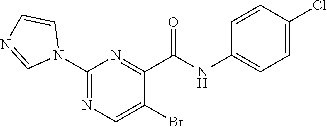 [SMILES: O=C(Nc1ccc(Cl)cc1)c1nc(-n2ccnc2)ncc1Br] | 5-bromo-N-(4-chlorophenyl)-2- (1H-imidazol-1-yl)pyrimidine-4- carboxamide |
| X-451 | 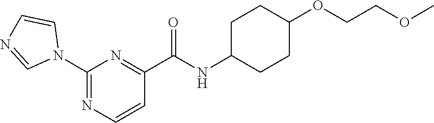 [SMILES: COCCOC1CCC(NC(=O)c2ccnc(-n3ccnc3)n2)CC1] | 2-(1H-imidazol-1-yl)-N-(4-(2- methoxyethoxy)cyclohexyl) pyrimidine-4-carboxamide |
| X-452 | 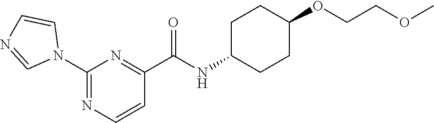 [SMILES: COCCO[C@H]1CC[C@H](NC(=O)c2ccnc(-n3ccnc3)n2)CC1] | 2-(1H-imidazol-1-yl)-N-((1r,4r)-4- (2- methoxyethoxy)cyclohexyl) pyrimidine-4-carboxamide |
| X-453 | 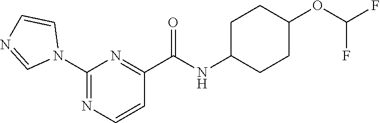 [SMILES: O=C(NC1CCC(OC(F)F)CC1)c1ccnc(-n2ccnc2)n1] | N-(4- (dilluoromethoxy)cyclohexyl)-2- (1H-imidazol-1-yl)pyrimidine-4- carboxamide |
| X-454 | 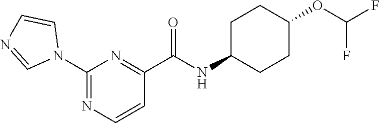 [SMILES: O=C(N[C@H]1CC[C@H](OC(F)F)CC1)c1ccnc(-n2ccnc2)n1] | N-((1r,4r)-4- (dilluoromethoxy)cyclohexyl)-2- (1H-imidazol-1-yl)pyrimidine-4- carboxamide |
| X-455 | 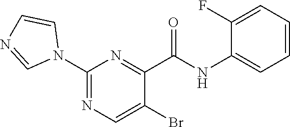 [SMILES: O=C(Nc1ccccc1F)c1nc(-n2ccnc2)ncc1Br] | 5-bromo-N-(2-fluorophenyl)-2- (1H-imidazol-1-yl)pyrimidine-4- carboxamide |
| X-456 | 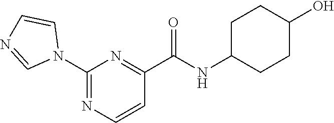 [SMILES: O=C(NC1CCC(O)CC1)c1ccnc(-n2ccnc2)n1] | N-(4-hydroxycyclohexyl)-2-(1H- imidazol-1-yl)pyrimidine-4- carboxamide |
| X-457 | 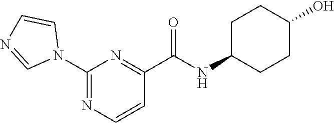 [SMILES: O=C(N[C@H]1CC[C@H](O)CC1)c1ccnc(-n2ccnc2)n1] | N-((1r,4r)-4-hydroxycyclohexyl)-2- (1H-imidazol-1-yl)pyrimidine-4- carboxamide |
| X-458 | 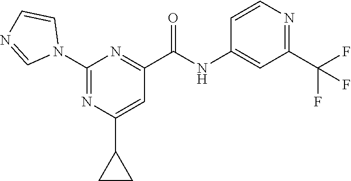 [SMILES: O=C(Nc1ccnc(C(F)(F)F)c1)c1cc(C2CC2)nc(-n2ccnc2)n1] | 6-cyclopropyl-2-(1H-imidazol-1- yl)-N-(2-(trifluoromethyl)pyridin- 4-yl)pyrimidine-4-carboxamide |
| X-459 | 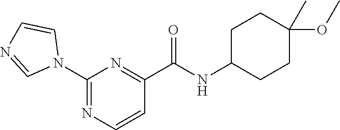 [SMILES: COC1(C)CCC(NC(=O)c2ccnc(-n3ccnc3)n2)CC1] | 2-(1H-imidazol-1-yl)-N-(4- methoxy-4- methylcyclohexyl)pyrimidine-4- carboxamide |
| X-460 | 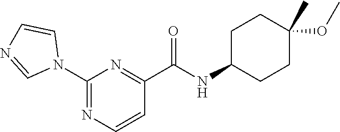 [SMILES: CO[C@]1(C)CC[C@@H](NC(=O)c2ccnc(-n3ccnc3)n2)CC1] | 2-(1H-imidazol-1-yl)-N-((1r,4r)-4- methoxy-4- methylcyclohexyl)pyrimidine-4- carboxamide |
| X-461 | 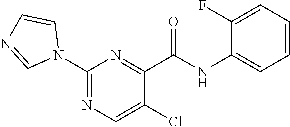 [SMILES: O=C(Nc1ccccc1F)c1nc(-n2ccnc2)ncc1Cl] | 5-chloro-N-(2-fluorophenyl)-2- (1H-imidazol-1-yl)pyrimidine-4- carboxamide |
| X-462 | 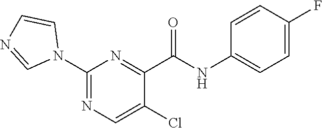 [SMILES: O=C(Nc1ccc(F)cc1)c1nc(-n2ccnc2)ncc1Cl] | 5-chloro-N-(4-fluorophenyl)-2- (1H-imidazol-1-yl)pyrimidine-4- carboxamide |
| X-463 | 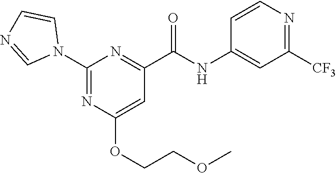 [SMILES: COCCOc1cc(C(=O)Nc2ccnc(C(F)(F)F)c2)nc(-n2ccnc2)n1] | 2-(1H-imidazol-1-yl)-6-(2- methoxy ethoxy)-N-(2- (trifluoromethyl)pyridin-4- yl)pyrimidine-4-carboxamide |
| X-464 | 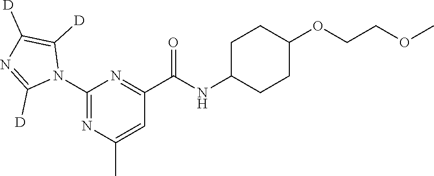 [SMILES: [2H]c1nc([2H])n(-c2nc(C)cc(C(=O)NC3CCC(OCCOC)CC3)n2)c1[2H]] | 2-(1H-imidazol-1-yl-d3)-N-(4-(2- methoxyethoxy)cyclohexyl)-6- methylpyrimidine-4-carboxamide |
| X-465 | 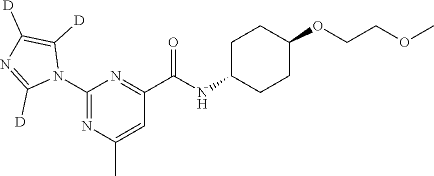 [SMILES: [2H]c1nc([2H])n(-c2nc(C)cc(C(=O)N[C@H]3CC[C@H](OCCOC)CC3)n2)c1[2H]] | 2-(1H-imidazol-1-yl-d3)-N-((1r,4r)- 4-(2-methoxyethoxy)cyclohexyl)-6- methylpyrimidine-4-carboxamide |
| X-466 | 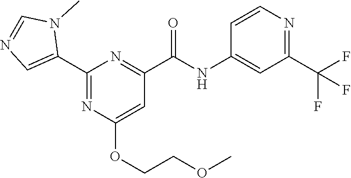 [SMILES: COCCOc1cc(C(=O)Nc2ccnc(C(F)(F)F)c2)nc(-c2cncn2C)n1] | 6-(2-methoxyethoxy)-2-(1-methyl- 1H-imidazol-5-yl)-N-(2- (trifluoromethyl)pyridin-4- yl)pyrimidine-4-carboxamide |

TABLE 6X

| Compound | | |
|---|---|---|
| Number | Structure | Name |
| X-467 | 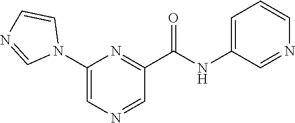 [SMILES: O=C(Nc1cccnc1)c1cncc(-n2ccnc2)n1] | 6-(1H-imidazol-1-yl)-N-(pyridin-3- yl)pyrazine-2-carboxamide |

In some embodiments, provided herein is a compound of formula (I):

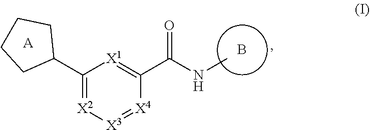

or a stereoisomer or tautomer thereof, or a pharmaceutically acceptable salt of any of the foregoing, wherein:

### X¹ is N or CH,

X² is N or C(R^{x}), wherein R^{x} is H, halo, or C₁₋₆alkyl,
X³ is N or C(R^{y}), wherein R^{y} is H, —OH, C₁₋₆alkoxy, C₃₋₁₀cycloalkyl, 3-10 membered heterocyclyl, or C₁₋₆alkyl,
wherein the C₁₋₆alkoxy of R^{y} is optionally substituted with one or more C₁₋₆alkoxy, the C₃₋₁₀cycloalkyl of R^{y} is optionally substituted with one or more halo, C₁₋₆alkoxy, or —OH, the 3-10 membered heterocyclyl of R^{y} is optionally substituted with one or more C₁₋₆alkyl, and the C₁₋₆alkyl of R^{y} is optionally substituted with one or more halo or —OH, and
   X⁴ is N or C(R^{z}), wherein R^{z} is H, halo, —NH₂, C₁₋₆alkoxy, or C₁₋₆alkyl, provided that at most two of X¹, X², X³, and X⁴ are N;

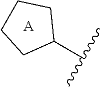

is:
(i)

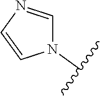 [SMILES: *n1ccnc1]

that is optionally substituted with one or more —C(O)—NH₂, or
(ii)

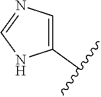 [SMILES: *c1cnc[nH]1]

that is optionally substituted with one or more C₁₋₆ alkyl, or
(iii)

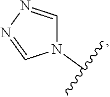 [SMILES: *n1cnnc1]

or
(iv)

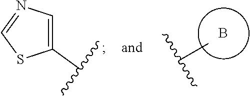

is:
(i) C₄₋₉cycloalkyl, wherein the C₄₋₉cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆ haloalkyl), phenyl, phenoxy, or pyridinyl,
wherein the C₁₋₆alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
   (ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆alkyl, —C(O)—C₁₋₆ alkoxy, or phenyl, wherein the phenyl of R^{b} is optionally substituted with one or more C₁₋₆haloalkyl, or
   (iii) phenyl, wherein the phenyl is optionally substituted with one or more halo, or with C₁₋₆alkyl optionally substituted with —OH, or
   (iv) pyridinyl, wherein the pyridinyl is optionally substituted with one or more halo, C₁₋₆haloalkyl, C₁₋₆alkoxy optionally substituted with one or more halo, C₁₋₆alkyl optionally substituted with —OH, or —O—C₃₋₁₀cycloalkyl optionally substituted with one or more halo; and further provided that one of (a), (b), (c), (d), (e), (f), (g), (h), (i), (j), or (k) applies:
(a) X¹ is CH, X² is C(R^{x}), X³ is C(R^{y}), and X⁴ is C(R^{z}), and further provided that one of (a-1), (a-2), or (a-3) applies:
   (a-1)

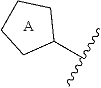

is

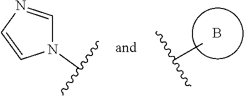

is pyridinyl;
(a-2)

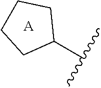

is

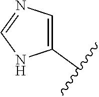 [SMILES: *c1cnc[nH]1]

that is substituted with one or more C₁₋₆ alkyl;
(a-3)

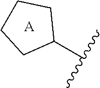

is

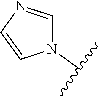 [SMILES: *n1ccnc1]

that is substituted with one or more —C(O)—NH₂,

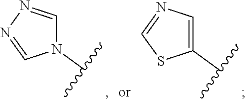 [SMILES: *n1cnnc1 ; *c1cncs1]

(b) X¹ is N, X² is C(R^{x}), X³ is C(R^{y}), and X⁴ is C(R^{z}), and provided that one of (b-1), (b-2), (b-3), or (b-4) applies:
   (b-1) when

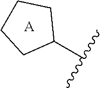

is

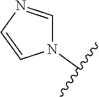 [SMILES: *n1ccnc1]

that is optionally substituted with one or more —C(O)—NH₂, then one of (b-1-i) or (b-1-ii) applies:
(b-1-i)

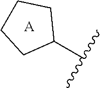

is

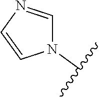 [SMILES: *n1ccnc1]

that is substituted with one or more —C(O)—NH₂;
(b-1-ii)

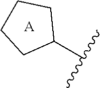

is

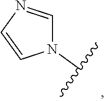 [SMILES: *n1ccnc1]

and provided that one of (b-1-ii-a), (b-1-ii-b), (b-1-ii-c), (b-1-ii-d), (b-1-ii-e), or (b-1-ii-f) applies:
   (b-1-ii-a) R^{y} is C₁₋₆alkyl, and

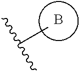

is:
   (i) C₄₋₉cycloalkyl, wherein the C₄₋₉cycloalkyl is optionally substituted with one or more R^{a}, wherein each R^{a} is independently —OH, halo, C₁₋₆alkyl, C₁₋₆haloalkyl, C₁₋₆ alkoxy, —C(O)—C₁₋₆ alkoxy, —NH(C₁₋₆ haloalkyl), phenyl, phenoxy, or pyridinyl,
   wherein the C₁₋₆alkoxy of R^{a} is optionally substituted with one or more halo or phenyl, the C₁₋₆alkyl of R^{a} is optionally substituted with one or more —OH or C₁₋₆ alkoxy, the phenyl of R^{a} is optionally substituted with one or more halo or C₁₋₆ alkoxy, and the pyridinyl of R^{a} is optionally substituted with one or more C₁₋₆haloalkyl; or
   (ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆ alkyl, —C(O)—C₁₋₆ alkoxy, or phenyl, wherein the phenyl of R^{b} is optionally substituted with one or more C₁₋₆haloalkyl; or
   (iii) phenyl, wherein the phenyl is optionally substituted with one or more halo, or with C₁₋₆alkyl optionally substituted with —OH; or
   (iv) pyridinyl, wherein the pyridinyl is substituted with one or more halo, C₁₋₆haloalkyl, C₁₋₆alkoxy optionally substituted with one or more halo, C₁₋₆alkyl optionally substituted with —OH, or —O—C₃₋₁₉cycloalkyl optionally substituted with one or more halo;
(b-1-ii-b) R^{y} is trifluoromethyl;
(b-1-ii-c) R^{y} is 3-10 membered heterocyclyl;
(b-1-ii-d) R^{y} is C₁₋₆alkoxy, and

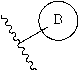

is:
   (i) C₄₋₉cycloalkyl, wherein the C₄₋₉cycloalkyl is optionally substituted with one or more R^{a}, wherein each R^{a} is independently —OH, halo, C₁₋₆alkyl, C₁₋₆haloalkyl, C₁₋₆ alkoxy, —C(O)—C₁₋₆ alkoxy, —NH(C₁₋₆ haloalkyl), phenyl, phenoxy, or pyridinyl,
   wherein the C₁₋₆alkoxy of R^{a} is optionally substituted with one or more halo or phenyl, the C₁₋₆alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl; or
   (ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆ alkyl, —C(O)—C₁₋₆ alkoxy, or phenyl, wherein the phenyl of R^{b} is optionally substituted with one or more C₁₋₆haloalkyl; or
   (iii) phenyl, wherein the phenyl is optionally substituted with one or more halo, or with C₁₋₆ alkyl optionally substituted with —OH; or
   (iv) 6-(difluoromethyl)pyridin-3-yl; or
   (v) 6-(trifluoromethyl)pyridin-3-yl;
(b-1-ii-e) R^{y} is C₁₋₆alkoxy substituted with one or more C₁₋₆alkoxy, and

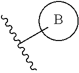

is:
   (i) C₄₋₉cycloalkyl, wherein the C₄₋₉cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆haloalkyl), phenyl, phenoxy, or pyridinyl,
   wherein the C₁₋₆alkoxy of R^{a} is optionally substituted with one or more halo, phenyl, or C₁₋₆alkoxy, the C₁₋₆alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆ haloalkyl; or
   (ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆ alkyl, —C(O)—C₁₋₆ alkoxy, or phenyl, wherein the phenyl of R^{b} is optionally substituted with one or more C₁₋₆ haloalkyl; or
   (iii) phenyl, wherein the phenyl is optionally substituted with one or more halo, or with C₁₋₆alkyl optionally substituted with —OH; or
   (iv) 6-(difluoromethyl)pyridin-3-yl; or
   (v) 6-(trifluoromethyl)pyridin-3-yl;
(b-1-ii-f) R^{y} is H, and provided that one of (b-1-ii-f-1), (b-1-ii-f-2), or (b-1-ii-f-3) applies:
   (b-1-ii-f-1) R^{z} is C₁₋₆alkyl, and

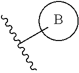

is:
(i) C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆ haloalkyl, C₁₋₆alkoxy, —C(O)—C₁₋₆alkoxy, —NH(C₁₋₆haloalkyl), phenyl, phenoxy, or pyridinyl,
wherein the C₁₋₆ alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl; or
(ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆ alkyl, —C(O)—C₁₋₆alkoxy, or phenyl, wherein the phenyl of R^{b} is optionally substituted with one or more C₁₋₆haloalkyl; or
(iii) phenyl, wherein the phenyl is optionally substituted with one or more halo, or with C₁₋₆ alkyl optionally substituted with —OH; or
(iv) pyridinyl substituted with one or more halo, C₁₋₆ haloalkyl, C₁₋₆alkoxy optionally substituted with one or more halo, C₁₋₆alkyl optionally substituted with —OH, or —O—C₃₋₁₀cycloalkyl optionally substituted with one or more halo;
(b-1-ii-f-2) R^{z} is halo, and

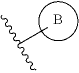

is:
(i) C₄₋₉ cycloalkyl, wherein the C₄₋₉cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆haloalkyl), phenyl, phenoxy, or pyridinyl,
wherein the C₁₋₆alkoxy of R^{a} is optionally substituted with one or more halo, phenyl, or C₁₋₆alkoxy, the C₁₋₆alkyl of R^{a} is optionally substituted with one or more —OH or C₁₋₆ alkoxy, the phenyl of R^{a} is optionally substituted with one or more halo or C₁₋₆ alkoxy, and the pyridinyl of R^{a} is optionally substituted with one or more C₁₋₆haloalkyl; or
(ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆alkyl, —C(O)—C₁₋₆alkoxy, or phenyl, wherein the phenyl of R^{b} is optionally substituted with one or more C₁₋₆haloalkyl; or
(iii) phenyl, wherein the phenyl is optionally substituted with one or more halo, or with C₁₋₆alkyl optionally substituted with —OH; or
(iv) pyridinyl substituted with one or more halo, C₁₋₆ haloalkyl, C₁₋₆alkoxy optionally substituted with one or more halo, C₁₋₆alkyl optionally substituted with —OH, or —O—C₃₋₁₀cycloalkyl optionally substituted with one or more halo;
(b-1-ii-f-3) R^{z} is H, and provided that one of (b-1-ii-f-3-i) or (b-1-ii-f-3-ii) applies:
(b-1-ii-f-3-i) R^{x} is halo, and

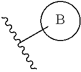

is:
(i) C₄₋₉ cycloalkyl, wherein the C₄₋₉cycloalkyl is optionally substituted with one or more R^{a}, wherein each R^{a} is independently —OH, halo, C₁₋₆ alkyl, C₁₋₆ haloalkyl, C₁₋₆ alkoxy, —C(O)—C₁₋₆alkoxy, —NH(C₁₋₆ haloalkyl), phenyl, phenoxy, or pyridinyl,
wherein the C₁₋₆alkoxy of R^{a} is optionally substituted with one or more halo, phenyl, or C₁₋₆alkoxy, the C₁₋₆alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl; or
(ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆alkyl, —C(O)—C₁₋₆ alkoxy, or phenyl, wherein the phenyl of R^{b} is optionally substituted with one or more C₁₋₆haloalkyl; or
(iii) phenyl, wherein the phenyl is optionally substituted with one or more halo, or with C₁₋₆alkyl optionally substituted with —OH; or
(iv) pyridinyl substituted with one or more halo, C₁₋₆ haloalkyl, C₁₋₆alkoxy optionally substituted with one or more halo, C₁₋₆alkyl optionally substituted with —OH, or —O—C₃₋₁₀cycloalkyl optionally substituted with one or more halo;
(b-1-ii-f-3-ii) R^{x} is H, and

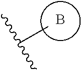

is:
(i) cyclobutyl optionally substituted with one or more R^{a}, wherein each R^{a} is independently —OH, halo, C₁₋₆alkyl, C₁₋₆haloalkyl, C₁₋₆alkoxy, —C(O)—C₁₋₆ alkoxy, —NH(C₁₋₆haloalkyl), phenyl, phenoxy, or pyridinyl,
wherein the C₁₋₆ alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl; or
(ii) cyclopentyl optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆ haloalkyl), phenyl, phenoxy, or pyridinyl,
wherein the C₁₋₆alkoxy of R^{a} is optionally substituted with one or more halo, phenyl, or C₁₋₆alkoxy, the C₁₋₆alkyl of R^{a} is optionally substituted with one or more C₁₋₆alkoxy, the phenyl of R^{a} is optionally substituted with one or more halo or C₁₋₆alkoxy, and the pyridinyl of R^{a} is optionally substituted with one or more C₁₋₆haloalkyl; or
(iii) cyclohexyl optionally substituted with one or more R^{a}, wherein each R^{a} is independently —OH, halo, C₁₋₆alkyl, C₁₋₆haloalkyl, C₁₋₆alkoxy, —C(O)—C₁₋₆ alkoxy, —NH(C₁₋₆haloalkyl), phenyl, phenoxy, or pyridinyl,
wherein the C₁₋₆alkoxy of R^{a} is substituted with one or more halo, phenyl, or C₁₋₆alkoxy, the C₁₋₆alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, and further provided that

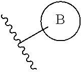

is not (1r,4r)-4-methoxycyclohexyl or (1r,4r)-4-hydroxycyclohexyl; or
(iv) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆alkyl, —C(O)—C₁₋₆ alkoxy, or phenyl, wherein the phenyl of R^{b} is optionally substituted with one or more C₁₋₆haloalkyl; or
(v) phenyl, wherein the phenyl is substituted with one or two fluoro; or
(vi) 2-methylpyridin-3-yl; or
(vii) 6-methylpyridin-2-yl; or
(viii) 2-(trifluoromethyl)pyridin-4-yl; or
(ix) 2-(difluoromethyl)pyridin-4-yl; or
(x) 6-(difluoromethyl)pyridin-3-yl; or
(xi) 4-methyl-6-trifluoromethyl-pyridin-3-yl;
(b-2) when

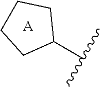

is

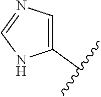 [SMILES: *c1cnc[nH]1]

that is optionally substituted with one or more C₁₋₆alkyl and

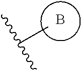

is pyridinyl, wherein the pyridinyl is optionally substituted with one or more halo, C₁₋₆haloalkyl, C₁₋₆alkoxy optionally substituted with one or more halo, C₁₋₆ alkyl optionally substituted with —OH, or —O-C₃₋₁₀cycloalkyl optionally substituted with one or more halo, then R^{y} is C₁₋₆ alkoxy;
(b-3) when

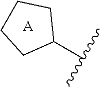

is

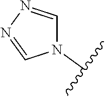 [SMILES: *n1cnnc1]

then

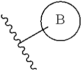

is pyridinyl, substituted with one or more C₁₋₆ haloalkyl;
(b-4) when

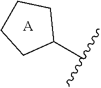

is

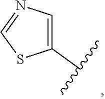 [SMILES: *c1cncs1]

and R^{y} is H, then

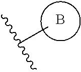

is:
   (i) C₄₋₉ cycloalkyl, wherein the C₄₋₉cycloalkyl is optionally substituted with one or more R^{a}, wherein each R^{a} is independently —OH, halo, C₁₋₆alkyl, C₁₋₆haloalkyl, —C(O)—C₁₋₆ alkoxy, —NH(C₁₋₆ haloalkyl), phenyl, phenoxy, or pyridinyl,
      wherein the C₁₋₆alkyl of R^{a} is optionally substituted with one or more —OH or C₁₋₆ alkoxy, the phenyl of R^{a} is optionally substituted with one or more halo or C₁₋₆ alkoxy, and the pyridinyl of R^{a} is optionally substituted with one or more C₁₋₆ haloalkyl, or
   (ii) 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆ alkoxy, or phenyl, wherein the phenyl of R^{b} is optionally substituted with one or more C₁₋₆haloalkyl, or
   (iii) phenyl, or
   (iv) pyridinyl, wherein the pyridinyl is optionally substituted with one or more halo, C₁₋₆alkoxy optionally substituted with one or more halo, C₁₋₆alkyl optionally substituted with —OH, or —O—C₃₋₁₀cycloalkyl optionally substituted with one or more halo;
(c) X¹ is CH, X² is N, X³ is C(R^{y}), and X⁴ is C(R^{z}), and provided that one of (c-1), (c-2), or (c-3) applies:
   (c-1)

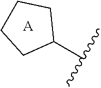

is

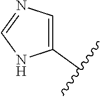 [SMILES: *c1cnc[nH]1]

that is optionally substituted with one or more C₁₋₆ alkyl;
(c-2)

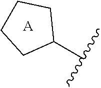

is

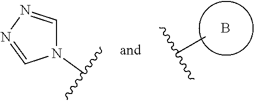

is:
   (i) C₄₋₉ cycloalkyl, wherein the C₄₋₉cycloalkyl is optionally substituted with one or more R^{a}, wherein each R^{a} is independently —OH, halo, C₁₋₆alkyl, C₁₋₆haloalkyl, C₁₋₆ alkoxy, —C(O)—C₁₋₆ alkoxy, —NH(C₁₋₆ haloalkyl), phenyl, phenoxy, or pyridinyl,
      wherein the C₁₋₆alkoxy of R^{a} is optionally substituted with one or more halo, phenyl, or C₁₋₆alkoxy, the C₁₋₆alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl; or
   (ii) 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆ alkoxy, or phenyl, wherein the phenyl of R^{b} is optionally substituted with one or more C₁₋₆haloalkyl; or
(c-3)

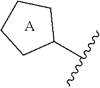

is

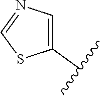 [SMILES: *c1cncs1]

and R^{y} is H;
(d) X¹ is CH, X² is C(R^{x}), X³ is N, and X⁴ is C(R^{z});
(e) X¹ is CH, X² is C(R^{x}), X³ is C(R^{y}), and X⁴ is N, and provided that when

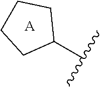

is

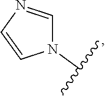 [SMILES: *n1ccnc1]

then

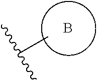

is not pyridin-3-yl;
(f) X¹ is N, X² is N, X³ is C(R^{y}), and X⁴ is C(R^{z}), and provided that one of (f-1), (f-2), (f-3), or (f-4) applies:

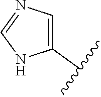 [SMILES: *c1cnc[nH]1]

(f-1) when

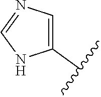 [SMILES: *c1cnc[nH]1]

is
   that is optionally substituted with one or more C₁₋₆alkyl and R^{y} is 2-methoxyethoxy, then

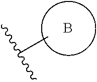

is not 2-trifluoromethylpyridin-4-yl;
(f-2) when

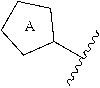

is

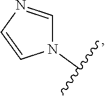 [SMILES: *n1ccnc1]

then one of (f-2-i), (f-2-ii), (f-2-iii), (f-2-iv), (f-2-v), or (f-2-vi) applies
   (f-2-i) R^{y} is cyclopropyl and

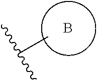

is:
   (i) C₄₋₉cycloalkyl, wherein the C₄₋₉cycloalkyl is optionally substituted with one or more R^{a}, wherein each R^{a} is independently —OH, halo, C₁₋₆alkyl, C₁₋₆ haloalkyl, C₁₋₆alkoxy, —C(O)—C₁₋₆alkoxy, —NH(C₁₋₆ haloalkyl), phenyl, phenoxy, or pyridinyl,
   wherein the C₁₋₆alkoxy of R^{a} is optionally substituted with one or more halo or phenyl, the C₁₋₆ alkyl of R^{a} is optionally substituted with one or more —OH or C₁₋₆ alkoxy, the phenyl of R^{a} is optionally substituted with one or more halo or C₁₋₆alkoxy, and the pyridinyl of R^{a} is optionally substituted with one or more C₁₋₆haloalkyl; or
   (ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆alkyl, —C(O)—C₁₋₆alkoxy, or phenyl, wherein the phenyl of R^{b} is optionally substituted with one or more C₁₋₆ haloalkyl; or
   (iii) phenyl, wherein the phenyl is optionally substituted with one or more halo, or with C₁₋₆alkyl optionally substituted with —OH; or
   (iv) pyridinyl, wherein the pyridinyl is optionally substituted with one or more halo, C₁₋₆alkoxy optionally substituted with one or more halo, C₁₋₆alkyl optionally substituted with —OH, or —O—C₃₋₁₉ cycloalkyl optionally substituted with one or more halo; or
   (v) 6-(difluoromethyl)pyridin-3-yl;
(f-2-ii) R^{y} is methyl and

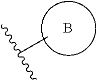

is:
   (i) C₄₋₉cycloalkyl, wherein the C₄₋₉cycloalkyl is optionally substituted with one or more R^{a}, wherein each R^{a} is independently —OH, halo, C₁₋₆alkyl, C₁₋₆ haloalkyl, C₁₋₆alkoxy, —C(O)—C₁₋₆alkoxy, —NH(C₁₋₆haloalkyl), phenyl, phenoxy, or pyridinyl,
   wherein the C₁₋₆alkyl of R^{a} is optionally substituted with one or more —OH or C₁₋₆ alkoxy, the phenyl of R^{a} is optionally substituted with one or more halo or C₁₋₆ alkoxy, and the pyridinyl of R^{a} is optionally substituted with one or more C₁₋₆haloalkyl, or
   (ii) 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆alkoxy, or phenyl, wherein the phenyl of R^{b} is optionally substituted with one or more C₁₋₆haloalkyl, or
   (iii) phenyl, wherein the phenyl is optionally substituted with one or more halo, or with C₁₋₆alkyl optionally substituted with —OH, or
   (iv) pyridinyl substituted with one or more halo, C₁₋₆ haloalkyl, C₁₋₆alkoxy optionally substituted with one or more halo, C₁₋₆alkyl optionally substituted with —OH, or —O—C₃₋₁₀cycloalkyl optionally substituted with one or more halo;
(f-2-iii) R^{y} is trifluoromethyl and

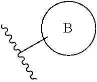

is:
   (i) C₄₋₉cycloalkyl, wherein the C₄₋₉cycloalkyl is optionally substituted with one or more R^{a}, wherein each R^{a} is independently —OH, halo, C₁₋₆alkyl, C₁₋₆ haloalkyl, C₁₋₆alkoxy, —C(O)—C₁₋₆alkoxy, —NH(C₁₋₆ haloalkyl), phenyl, phenoxy, or pyridinyl,
   wherein the C₁₋₆alkoxy of R^{a} is optionally substituted with one or more halo or phenyl, the C₁₋₆alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
   (ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆alkyl, —C(O)—C₁₋₆alkoxy, or phenyl, wherein the phenyl of R^{b} is optionally substituted with one or more C₁₋₆ haloalkyl, or
   (iii) phenyl, wherein the phenyl is optionally substituted with one or more halo, or with C₁₋₆alkyl optionally substituted with —OH, or
   (iv) pyridinyl, wherein the pyridinyl is optionally substituted with one or more halo, C₁₋₆ haloalkyl, C₁₋₆ alkoxy optionally substituted with one or more halo, C₁₋₆ alkyl optionally substituted with —OH, or —O—C₃₋₁₀cycloalkyl optionally substituted with one or more halo;
(f-2-iv) R^{y} is methoxy and

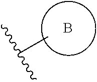

is:
   (i) C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆ haloalkyl, C₁₋₆alkoxy, —C(O)—C₁₋₆ alkoxy, —NH(C₁₋₆haloalkyl), phenyl, phenoxy, or pyridinyl,
   wherein the C₁₋₆ alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
   (ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆alkoxy, or phenyl, wherein the phenyl of R^{b} is optionally substituted with one or more C₁₋₆haloalkyl, or
   (iii) phenyl, wherein the phenyl is optionally substituted with one or more halo, or with C₁₋₆alkyl optionally substituted with —OH, or
   (iv) pyridinyl, wherein the pyridinyl is optionally substituted with one or more halo, C₁₋₆alkoxy optionally substituted with one or more halo, C₁₋₆alkyl optionally substituted with —OH, or —O—C₃₋₁₀ cycloalkyl optionally substituted with one or more halo; or
   (v) 6-(difluoromethyl)pyridin-3-yl;
(f-2-v) R^{y} is —OH, C₂₋₆alkoxy, C₄₋₁₀cycloalkyl, 3-10 membered heterocyclyl, or C₂₋₆alkyl,
   wherein the C₂₋₆ alkoxy of R^{y} is optionally substituted with one or more C₁₋₆alkoxy, the C₄₋₁₀cycloalkyl of R^{y} is optionally substituted with one or more halo, C₁₋₆alkoxy, or —OH, the 3-10 membered heterocyclyl of R^{y} is optionally substituted with one or more C₁₋₆alkyl, and the C₂₋₆alkyl of R^{y} is optionally substituted with one or more halo or —OH;
(f-2-vi) R^{y} is H, R^{z} is H, and

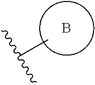

is:
   (i) C₄₋₉ cycloalkyl, wherein the C₄₋₉cycloalkyl is optionally substituted with one or more R^{a}, wherein each R^{a} is independently halo, C₁₋₆alkyl, C₁₋₆haloalkyl, —C(O)—C₁₋₆ alkoxy, —NH(C₁₋₆haloalkyl), phenyl, phenoxy, or pyridinyl,
   wherein the C₁₋₆ alkyl of R^{a} is optionally substituted with one or more —OH or C₁₋₆ alkoxy, the phenyl of R^{a} is optionally substituted with one or more halo or C₁₋₆alkoxy, and the pyridinyl of R^{a} is optionally substituted with one or more C₁₋₆haloalkyl, or
   (ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C₁₋₆ alkyl, —C(O)—C₁₋₆alkoxy, or phenyl, wherein the phenyl of R^{b} is optionally substituted with one or more C₁₋₆haloalkyl, or
   (iii) phenyl, wherein the phenyl is optionally substituted with one or more halo, or with C₁₋₆alkyl optionally substituted with —OH, or
   (iv) pyridinyl, wherein the pyridinyl is optionally substituted with one or more halo, C₁₋₆haloalkyl, C₁₋₆alkoxy optionally substituted with one or more halo, C₁₋₆alkyl optionally substituted with —OH, or —O—C₃₋₁₀cycloalkyl optionally substituted with one or more halo;
(f-3)

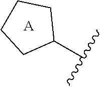

is

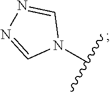 [SMILES: *n1cnnc1]

(f-4)

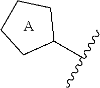

is

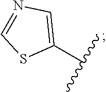 [SMILES: *c1cncs1]

(g) X¹ is N, X² is C(R^{x}), X³ is N, and X⁴ is C(R^{z}), and provided that one of (g-1), (g-2), (g-3), or (g-4) applies:
   (g-1)

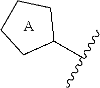

is

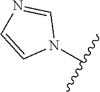 [SMILES: *n1ccnc1]

that is optionally substituted with one or more —C(O)—NH₂, and

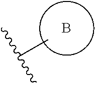

is:
   (i) C₄₋₉ cycloalkyl, wherein the C₄₋₉cycloalkyl is optionally substituted with one or more R^{a}, wherein each R^{a} is independently —OH, halo, C₁₋₆alkyl, C₁₋₆haloalkyl, C₁₋₆ alkoxy, —C(O)—C₁₋₆ alkoxy, —NH(C₁₋₆ haloalkyl), phenyl, phenoxy, or pyridinyl,
      wherein the C₁₋₆alkoxy of R^{a} is optionally substituted with one or more halo, phenyl, or C₁₋₆alkoxy, the C₁₋₆alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
   (ii) 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆ alkoxy, or phenyl, wherein the phenyl of R^{b} is optionally substituted with one or more C₁₋₆haloalkyl, or
   (iii) phenyl, wherein the phenyl is optionally substituted with one or more halo, or with C₁₋₆alkyl optionally substituted with —OH, or
   (iv) pyridinyl substituted with one or more halo, C₁₋₆haloalkyl, C₁₋₆alkoxy optionally substituted with one or more halo, C₁₋₆ alkyl optionally substituted with —OH, or —O—C₃₋₁₀ cycloalkyl optionally substituted with one or more halo;
(g-2)

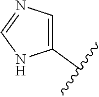 [SMILES: *c1cnc[nH]1]

that is optionally substituted with one or more C₁₋₆ alkyl;
(g-3)

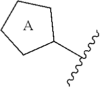

is

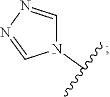 [SMILES: *n1cnnc1]

(g-4)

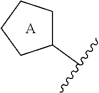

is

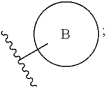

(h) X¹ is N, X² is C(R^{x}), X³ is C(R^{y}), and X⁴ is N;
(i) X¹ is CH, X² is N, X³ is N, and X⁴ is C(R^{z});
(j) X¹ is CH, X² is N, X³ is C(R^{y}), and X⁴ is N;
(k) X¹ is CH, X² is C(R^{x}), X³ is N, and X⁴ is N.

In some embodiments, provided herein is a compound of formula (I):

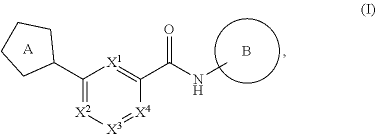

or a stereoisomer or tautomer thereof, or a pharmaceutically acceptable salt of any of the foregoing, wherein:

### X¹ is N or CH,

X² is N or C(R^{x}), wherein R^{x} is H, halo, or C₁₋₆alkyl,
X³ is N or C(R^{y}), wherein R^{y} is H, —OH, C₁₋₆alkoxy, C₃₋₁₀cycloalkyl, 3-10 membered heterocyclyl, or C₁₋₆alkyl, wherein the C₁₋₆alkyl of R^{y} is optionally substituted with one or more halo or —OH, and
X⁴ is N or C(R^{z}), wherein R^{z} is H, halo, or C₁₋₆alkyl, provided that at most two of X¹, X², X³, and X⁴ are N;

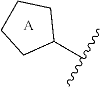

is:
(i)

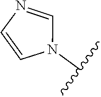 [SMILES: *n1ccnc1]

that is optionally substituted with one or more —C(O)—NH₂, or
(ii)

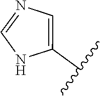 [SMILES: *c1cnc[nH]1]

that is optionally substituted with one or more C₁₋₆ alkyl, or
(iii)

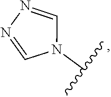 [SMILES: *n1cnnc1]

or
(iv)

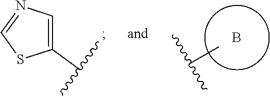

is:
(i) saturated C₄₋₈cycloalkyl, wherein the C₄₋₈cycloalkyl is optionally substituted with one or more R^{a}, wherein each R^{a} is independently —OH, halo, C₁₋₆alkyl, C₁₋₆haloalkyl, C₁₋₆alkoxy, or —C(O)—C₁₋₆ alkoxy, wherein the C₁₋₆ alkoxy of R^{a} is optionally substituted with one or more C₁₋₆alkoxy and the C₁₋₆ alkyl of R^{a} is optionally substituted with one or more —OH or C₁₋₆alkoxy, or
(ii) saturated 4-8 membered heterocyclyl, wherein the 4-8 membered heterocyclyl is optionally substituted with one or more R^{b}, wherein each R^{b} is independently oxo, —C(O)—C₁₋₆alkyl, —C(O)—C₁₋₆ alkoxy, or phenyl, wherein the phenyl of R^{b} is optionally substituted with one or more C₁₋₆haloalkyl, or
(iii) phenyl, wherein the phenyl is optionally substituted with one or more halo, or
(iv) pyridinyl, wherein the pyridinyl is optionally substituted with one or more halo, C₁₋₆alkyl, C₁₋₆haloalkyl, or C₁₋₆ alkoxy.

In some embodiments, at most two of X¹, X², X³, and X⁴ are N and at most three of X¹, X², X³, and X⁴ are not N.

In some embodiments, provided herein is a compound of formula (I), or a stereoisomer or tautomer thereof, or a pharmaceutically acceptable salt of any of the foregoing, wherein exactly two of X¹, X², X³, and X⁴ are N.

In some embodiments, provided herein is a compound of formula (I), or a stereoisomer or tautomer thereof, or a pharmaceutically acceptable salt of any of the foregoing, wherein X¹ is N, X² is N, X³ is C(R^{y}), and X⁴ is C(R^{z}). In some embodiments, provided herein is a compound of formula (I), or a stereoisomer or tautomer thereof, or a pharmaceutically acceptable salt of any of the foregoing, wherein the compound is of formula (I-A1):

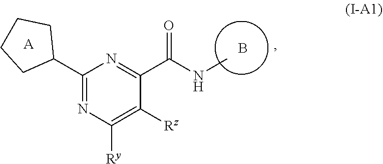

or a stereoisomer or tautomer thereof, or a pharmaceutically acceptable salt of any of the foregoing, wherein

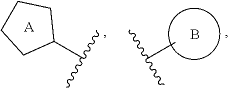

R^{y}, and R^{z} are as defined for a compound of formula (I). In some embodiments, provided herein is a compound of formula (I), or a stereoisomer or tautomer thereof, or a pharmaceutically acceptable salt of any of the foregoing, wherein the compound is of formula (I-A1):

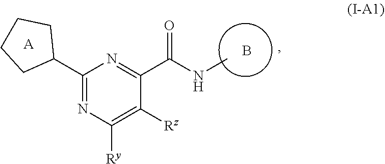

or a stereoisomer or tautomer thereof, or a pharmaceutically acceptable salt of any of the foregoing. In some embodiments of the foregoing, the compound of formula (I-A1) is

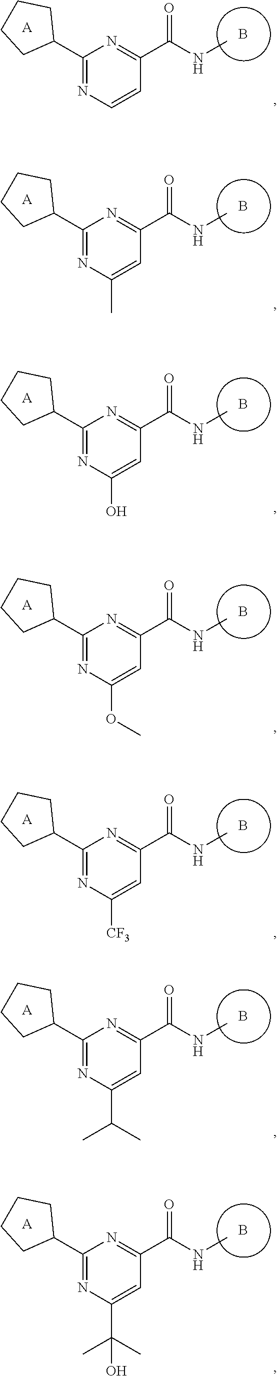

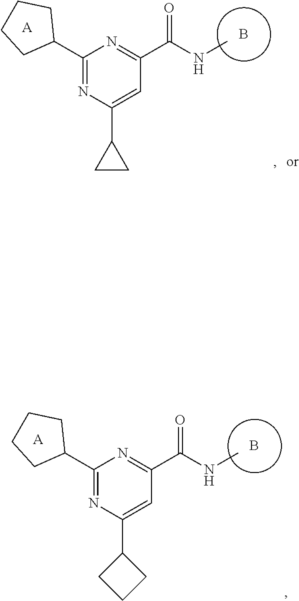

or a stereoisomer or tautomer thereof, or a pharmaceutically acceptable salt of any of the foregoing.

In some embodiments of formula (I-A1), the compound of formula (I-A1) or the stereoisomer or tautomer thereof, or the pharmaceutically acceptable salt of any of the foregoing, is not a compound selected from the compounds of Table 5X or a stereoisomer or tautomer thereof, or a pharmaceutically acceptable salt of any of the foregoing.

In some embodiments of formula (I-A1), R^{y} is cyclohexyl or 3-10 membered heterocyclyl, wherein the cyclohexyl is optionally substituted with one or more halo, C₁₋₆alkoxy, or —OH, and the 3-10 membered heterocyclyl is optionally substituted with one or more C₁₋₆alkyl;

R^{z} is H, halo, —NH₂, C₁₋₆alkoxy, or C₁₋₆alkyl;

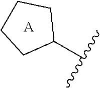

is:
(i)

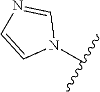 [SMILES: *n1ccnc1]

that is optionally substituted with one or more —C(O)—NH₂, or
(ii)

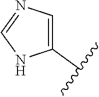 [SMILES: *c1cnc[nH]1]

that is optionally substituted with one or more C₁₋₆ alkyl, or
(iii)

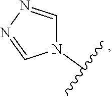 [SMILES: *n1cnnc1]

or
(iv)

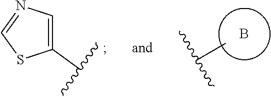

is:
(i) C₄₋₉cycloalkyl, wherein the C₄₋₉cycloalkyl is optionally substituted with one or more R^{a}, wherein each R^{a} is independently —OH, halo, C₁₋₆alkyl, C₁₋₆alkoxy, —C(O)—C₁₋₆alkoxy, —NH(C₁₋₆haloalkyl), phenyl, phenoxy, or pyridinyl,
wherein the C₁₋₆alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
   (ii) 4-9 membered heterocyclyl, wherein the 4-9 membered heterocyclyl is optionally substituted with one or more R^{b}, wherein each R^{b} is independently halo, C₁₋₆alkyl, oxo, —C(O)—C(O)—C₁₋₆ alkoxy, or phenyl, wherein the phenyl of R^{b} is optionally substituted with one or more C₁₋₆haloalkyl, or
   (iii) phenyl, wherein the phenyl is optionally substituted with one or more halo, or with C₁₋₆alkyl optionally substituted with —OH, or
   (iv) pyridinyl, wherein the pyridinyl is optionally substituted with one or more halo, C₁₋₆haloalkyl, C₁₋₆alkoxy optionally substituted with one or more halo, C₁₋₆alkyl optionally substituted with —OH, or —O—C₃₋₁₀cycloalkyl optionally substituted with one or more halo.

In some embodiments of formula (I), provided herein is a compound of formula (I), or a stereoisomer or tautomer thereof, or a pharmaceutically acceptable salt of any of the foregoing, wherein X¹ is N, X² is C(R^{x}), X³ is C(R^{y}), and X⁴ is N. In some embodiments, provided herein is a compound of formula (I), or a stereoisomer or tautomer thereof, or a pharmaceutically acceptable salt of any of the foregoing, wherein the compound is of formula (I-A2):

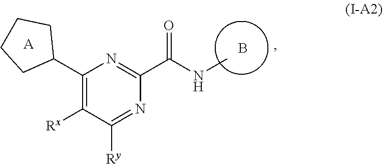

or a stereoisomer or tautomer thereof, or a pharmaceutically acceptable salt of any of the foregoing, wherein

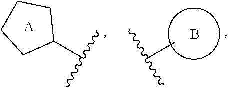

R^{x}, and R^{y} are as defined for a compound of formula (I). In some embodiments, provided herein is a compound of formula (I), or a stereoisomer or tautomer thereof, or a pharmaceutically acceptable salt of any of the foregoing, wherein the compound is of formula (I-A2):

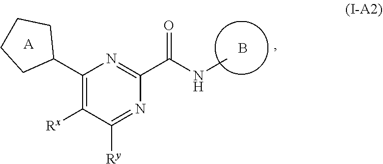

or a stereoisomer or tautomer thereof, or a pharmaceutically acceptable salt of any of the foregoing. In some embodiments of the foregoing, the compound of formula (I-A2) is

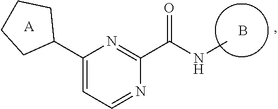

or a stereoisomer or tautomer thereof, or a pharmaceutically acceptable salt of any of the foregoing.

In some embodiments, X¹ is N, X² is C(R^{x}), X³ is N, and X⁴ is C(R^{z}). In some embodiments, provided herein is a compound of formula (I), or a stereoisomer or tautomer thereof, or a pharmaceutically acceptable salt of any of the foregoing, wherein the compound is of formula (I-A3):

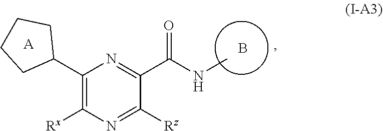

or a stereoisomer or tautomer thereof, or a pharmaceutically acceptable salt of any of the foregoing, wherein

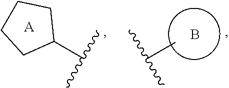

R^{x}, and R^{z} are as defined for a compound of formula (I). In some embodiments, provided herein is a compound of formula (I), or a stereoisomer or tautomer thereof, or a pharmaceutically acceptable salt of any of the foregoing, wherein the compound is of formula (I-A3):

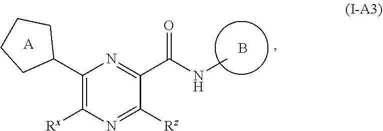

or a stereoisomer or tautomer thereof, or a pharmaceutically acceptable salt of any of the foregoing. In some embodiments of the foregoing, the compound of formula (I-A3) is

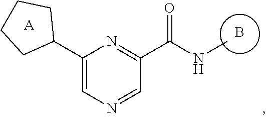

or a stereoisomer or tautomer thereof, or a pharmaceutically acceptable salt of any of the foregoing.

In some embodiments of formula (I-A3), the compound of formula (I-A3) or the stereoisomer or tautomer thereof, or the pharmaceutically acceptable salt of any of the foregoing, is not a compound selected from the compounds of Table 6X or a stereoisomer or tautomer thereof, or a pharmaceutically acceptable salt of any of the foregoing.

In some embodiments, provided herein is a compound of formula (I), or a stereoisomer or tautomer thereof, or a pharmaceutically acceptable salt of any of the foregoing, wherein exactly one of X¹, X², X³, and X⁴ is N.

In some embodiments, provided herein is a compound of formula (I), or a stereoisomer or tautomer thereof, or a pharmaceutically acceptable salt of any of the foregoing, wherein X¹ is CH, X² is C(R^{x}), X³ is C(R^{y}), and X⁴ is N. In some embodiments, provided herein is a compound of formula (I), or a stereoisomer or tautomer thereof, or a pharmaceutically acceptable salt of any of the foregoing, wherein the compound is of formula (I-B1):

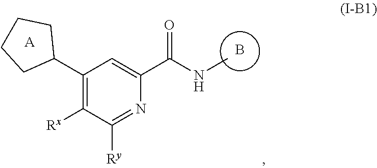

or a stereoisomer or tautomer thereof, or a pharmaceutically acceptable salt of any of the foregoing, wherein

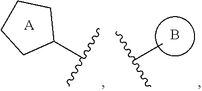

R^{x}, and R^{y} are as defined for a compound of formula (I). In some embodiments, provided herein is a compound of formula (I), or a stereoisomer or tautomer thereof, or a pharmaceutically acceptable salt of any of the foregoing, wherein the compound is of formula (I-B1):

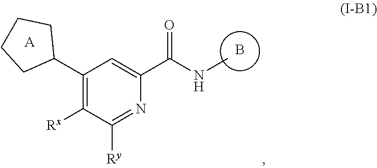

or a stereoisomer or tautomer thereof, or a pharmaceutically acceptable salt of any of the foregoing. In some embodiments of the foregoing, the compound of formula (I-B1) is

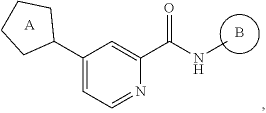

or a stereoisomer or tautomer thereof, or a pharmaceutically acceptable salt of any of the foregoing.

In some embodiments of formula (I-B1), the compound of formula (I-B1) or the stereoisomer or tautomer thereof, or the pharmaceutically acceptable salt of any of the foregoing, is not a compound selected from the compounds of Table 4X or a stereoisomer or tautomer thereof, or a pharmaceutically acceptable salt of any of the foregoing.

In some embodiments, provided herein is a compound of formula (I), or a stereoisomer or tautomer thereof, or a pharmaceutically acceptable salt of any of the foregoing, wherein X¹ is CH, X² is N, X³ is C(R^{y}), and X⁴ is C(R^{z}). In some embodiments, provided herein is a compound of formula (I), or a stereoisomer or tautomer thereof, or a pharmaceutically acceptable salt of any of the foregoing, wherein the compound is of formula (I-B2):

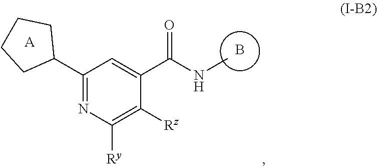

or a stereoisomer or tautomer thereof, or a pharmaceutically acceptable salt of any of the foregoing, wherein

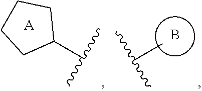

R^{y}, and R^{z} are as defined for a compound of formula (I). In some embodiments, provided herein is a compound of formula (I), or a stereoisomer or tautomer thereof, or a pharmaceutically acceptable salt of any of the foregoing, wherein the compound is of formula (I-B2):

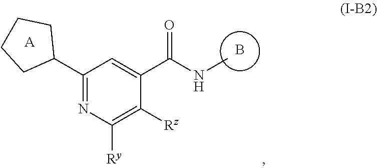

or a stereoisomer or tautomer thereof, or a pharmaceutically acceptable salt of any of the foregoing. In some embodiments of the foregoing, the compound of formula (I-B2) is

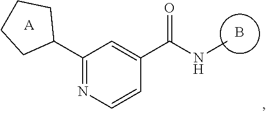

or a stereoisomer or tautomer thereof, or a pharmaceutically acceptable salt of any of the foregoing.

In some embodiments of formula (I-B2), the compound of formula (I-B2) or the stereoisomer or tautomer thereof, or the pharmaceutically acceptable salt of any of the foregoing, is not a compound selected from the compounds of Table 3X or a stereoisomer or tautomer thereof, or a pharmaceutically acceptable salt of any of the foregoing.

In some embodiments, provided herein is a compound of formula (I), or a stereoisomer or tautomer thereof, or a pharmaceutically acceptable salt of any of the foregoing, wherein X¹ is N, X² is C(R^{x}), X³ is C(R^{y}), and X⁴ is C(R^{z}). In some embodiments, provided herein is a compound of formula (I), or a stereoisomer or tautomer thereof, or a pharmaceutically acceptable salt of any of the foregoing, wherein the compound is of formula (I-B3):

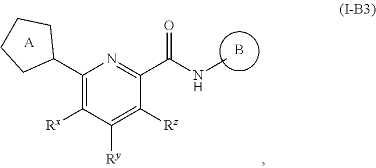

or a stereoisomer or tautomer thereof, or a pharmaceutically acceptable salt of any of the foregoing, wherein

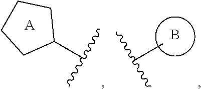

R^{x}, R^{y}, and R^{z} are as defined for a compound of formula (I). In some embodiments, provided herein is a compound of formula (I), or a stereoisomer or tautomer thereof, or a pharmaceutically acceptable salt of any of the foregoing, wherein the compound is of formula (I-B3):

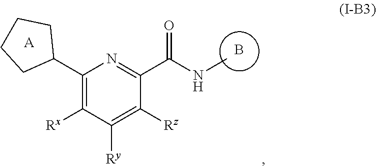

or a stereoisomer or tautomer thereof, or a pharmaceutically acceptable salt of any of the foregoing. In some embodiments of the foregoing, the compound of formula (I-B3) is

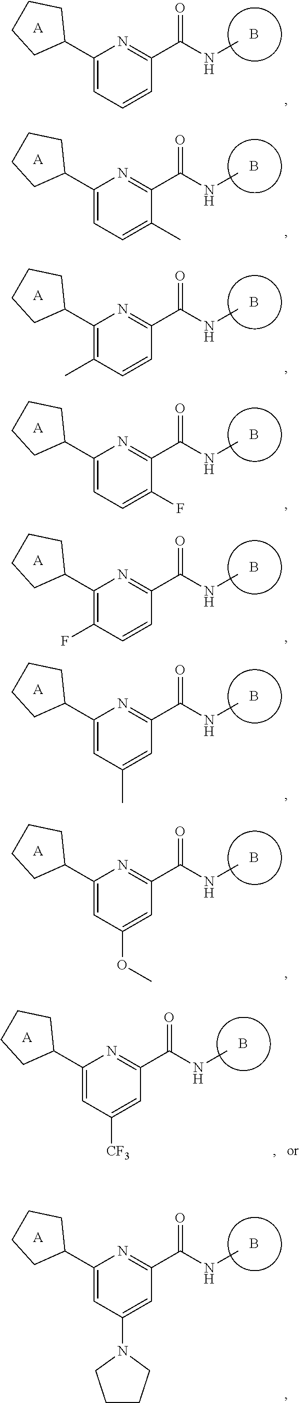

or a stereoisomer or tautomer thereof, or a pharmaceutically acceptable salt of any of the foregoing.

In some embodiments of formula (I-B3), the compound of formula (I-B3) or the stereoisomer or tautomer thereof, or the pharmaceutically acceptable salt of any of the foregoing, is not a compound selected from the compounds of Table 2X or a stereoisomer or tautomer thereof, or a pharmaceutically acceptable salt of any of the foregoing.

In some embodiments, provided herein is a compound of formula (I), or a stereoisomer or tautomer thereof, or a pharmaceutically acceptable salt of any of the foregoing, wherein X¹ is CH, X² is C(R^{x}), X³ is C(R^{y}), and X⁴ is C(R^{z}). In some embodiments, provided herein is a compound of formula (I), or a stereoisomer or tautomer thereof, or a pharmaceutically acceptable salt of any of the foregoing, wherein the compound is of formula (I-C):

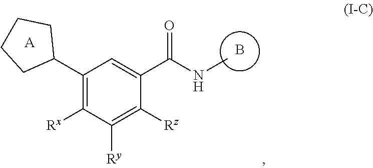

or a stereoisomer or tautomer thereof, or a pharmaceutically acceptable salt of any of the foregoing, wherein

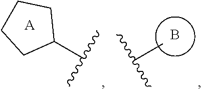

R^{x}, R^{y}, and R^{z} are as defined for a compound of formula (I). In some embodiments, provided herein is a compound of formula (I), or a stereoisomer or tautomer thereof, or a pharmaceutically acceptable salt of any of the foregoing, wherein the compound is of formula (I-C):

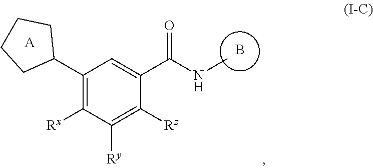

or a stereoisomer or tautomer thereof, or a pharmaceutically acceptable salt of any of the foregoing. In some embodiments of the foregoing, the compound of formula (I-C) is

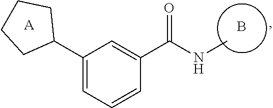

or a pharmaceutically acceptable salt of any of the foregoing. In some embodiments of the foregoing.

In some embodiments of formula (I-C), the compound of formula (I-C) or the stereoisomer or tautomer thereof, or the pharmaceutically acceptable salt of any of the foregoing, is not a compound selected from the compounds of Table 1X or a stereoisomer or tautomer thereof, or a pharmaceutically acceptable salt of any of the foregoing.

In some embodiments, provided herein is a compound of formula (I), such as a compound of formula (I-A1), (I-A2), (I-A3), (I-B1), (I-B2), (I-B3), or (I-C), or a stereoisomer or tautomer thereof, or a pharmaceutically acceptable salt of any of the foregoing, wherein

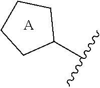

is

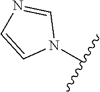 [SMILES: *n1ccnc1]

that is optionally substituted with one or more —C(O)—NH₂. In some embodiments of the foregoing,

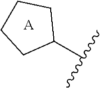

is

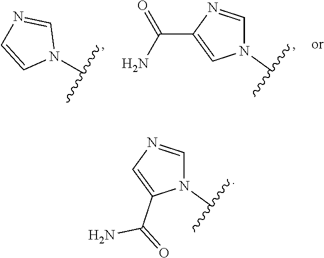 [SMILES: *n1ccnc1 ; *n1cnc(C(N)=O)c1 ; *n1cncc1C(N)=O]

In some embodiments of the foregoing,

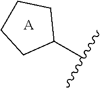

is

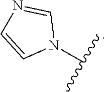 [SMILES: *n1ccnc1]

In some embodiments of the foregoing,

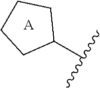

is

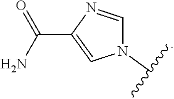 [SMILES: *n1cnc(C(N)=O)c1]

In some embodiments of the foregoing,

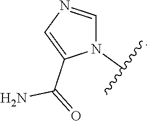 [SMILES: *n1cncc1C(N)=O]

In some embodiments, provided herein is a compound of formula (I), or a stereoisomer or tautomer thereof, or a pharmaceutically acceptable salt of any of the foregoing, wherein the compound is of formula (I-D):

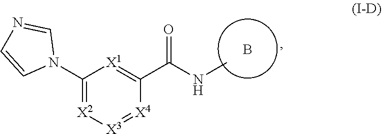

or a stereoisomer or tautomer thereof, or a pharmaceutically acceptable salt of any of the foregoing.

In some embodiments, provided herein is a compound of formula (I), or a stereoisomer or tautomer thereof, or a pharmaceutically acceptable salt of any of the foregoing, wherein the compound is of formula (I-E):

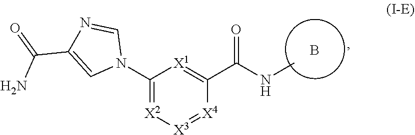

or a stereoisomer or tautomer thereof, or a pharmaceutically acceptable salt of any of the foregoing.

In some embodiments, provided herein is a compound of formula (I), or a stereoisomer or tautomer thereof, or a pharmaceutically acceptable salt of any of the foregoing, wherein the compound is of formula (I-F):

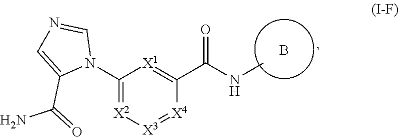

or a stereoisomer or tautomer thereof, or a pharmaceutically acceptable salt of any of the foregoing.

In some embodiments, provided herein is a compound of formula (I), such as a compound of formula (I-A1), (I-A2), (I-A3), (I-B1), (I-B2), (I-B3), or (I-C), or a stereoisomer or tautomer thereof, or a pharmaceutically acceptable salt of any of the foregoing, wherein

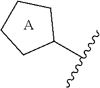

is

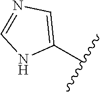 [SMILES: *c1cnc[nH]1]

that is optionally substituted with one or more C₁₋₆ alkyl. In some embodiments,

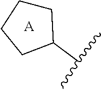

is

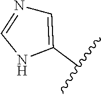 [SMILES: *c1cnc[nH]1]

that is optionally substituted with one or more methyl. In some embodiments of the foregoing,

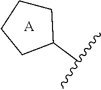

is

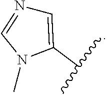 [SMILES: *c1cncn1C]

In some embodiments of the foregoing, provided herein is a compound of formula (I), or a stereoisomer or tautomer thereof, or a pharmaceutically acceptable salt of any of the foregoing, wherein the compound is of formula (I-G):

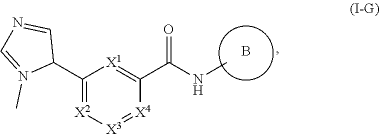

or a stereoisomer or tautomer thereof, or a pharmaceutically acceptable salt of any of the foregoing.

In some embodiments, provided herein is a compound of formula (I), such as a compound of formula (I-A1), (I-A2), (I-A3), (I-B1), (I-B2), (I-B3), or (I-C), or a stereoisomer or tautomer thereof, or a pharmaceutically acceptable salt of any of the foregoing, wherein

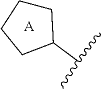

is

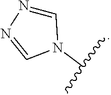 [SMILES: *n1cnnc1]

In some embodiments of the foregoing, provided herein is a compound of formula (I), or a stereoisomer or tautomer thereof, or a pharmaceutically acceptable salt of any of the foregoing, wherein the compound is of formula (I-H):

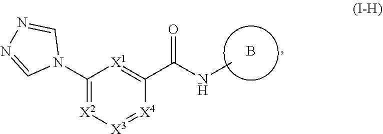

or a stereoisomer or tautomer thereof, or a pharmaceutically acceptable salt of any of the foregoing.

In some embodiments, provided herein is a compound of formula (I), such as a compound of formula (I-A1), (I-A2), (I-A3), (I-B1), (I-B2), (I-B3), or (I-C), or a stereoisomer or tautomer thereof, or a pharmaceutically acceptable salt of any of the foregoing, wherein

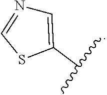 [SMILES: *c1cncs1]

is

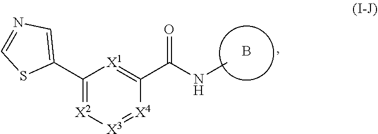

In some embodiments of the foregoing, provided herein is a compound of formula (I), or a stereoisomer or tautomer thereof, or a pharmaceutically acceptable salt of any of the foregoing, wherein the compound is of formula (I-J):

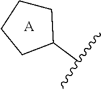

or a stereoisomer or tautomer thereof, or a pharmaceutically acceptable salt of any of the foregoing.

In some embodiments, provided herein is a compound of formula (I), such as a compound of formula (I-A1), (I-A2), (I-A3), (I-B1), (I-B2), (I-B3), (I-C), (I-D), (I-E), (I-F), (I-G), (I-H), or (I-J), or a stereoisomer or tautomer thereof, or a pharmaceutically acceptable salt of any of the foregoing, wherein

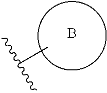

is C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆ haloalkyl, C₁₋₆ alkoxy, —C(O)—C₁₋₆ alkoxy, —NH(C₁₋₆ haloalkyl), phenyl, phenoxy, or pyridinyl, wherein the C₁₋₆alkoxy of R^{a} is optionally substituted with one or more halo, phenyl, or C₁₋₆alkoxy, the C₁₋₆alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl. In some embodiments,

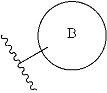

is

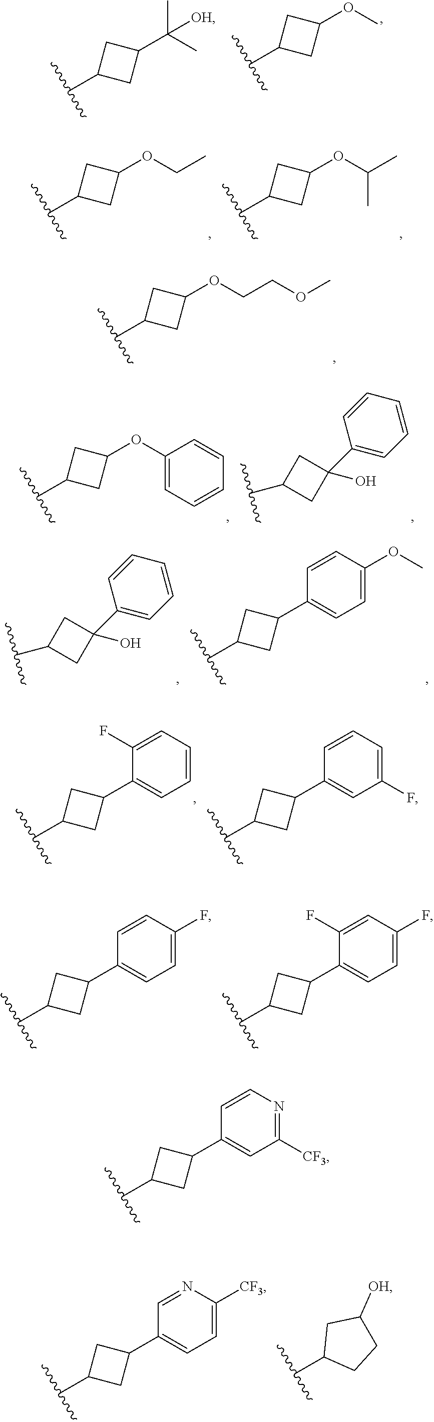

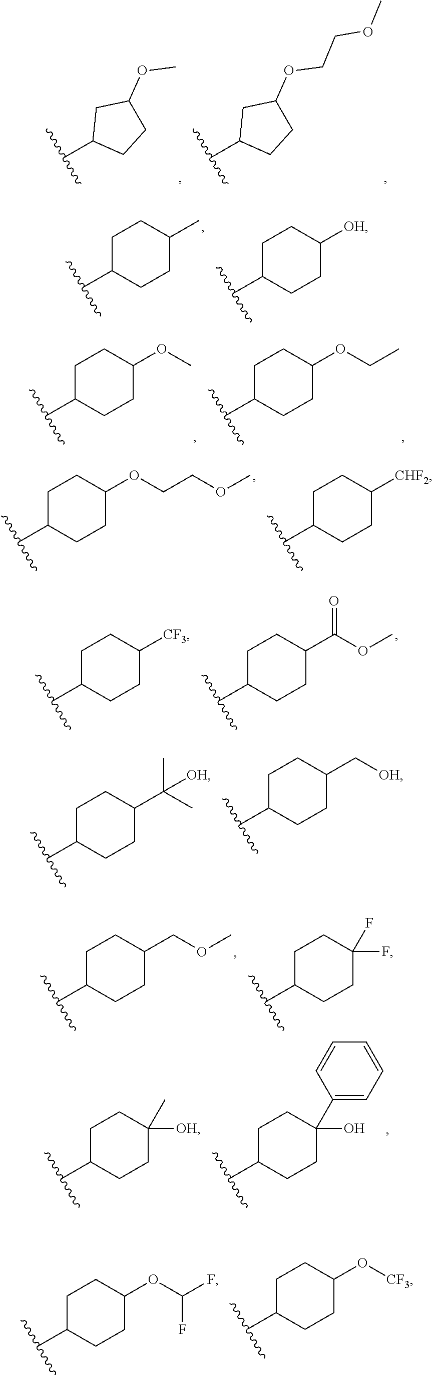

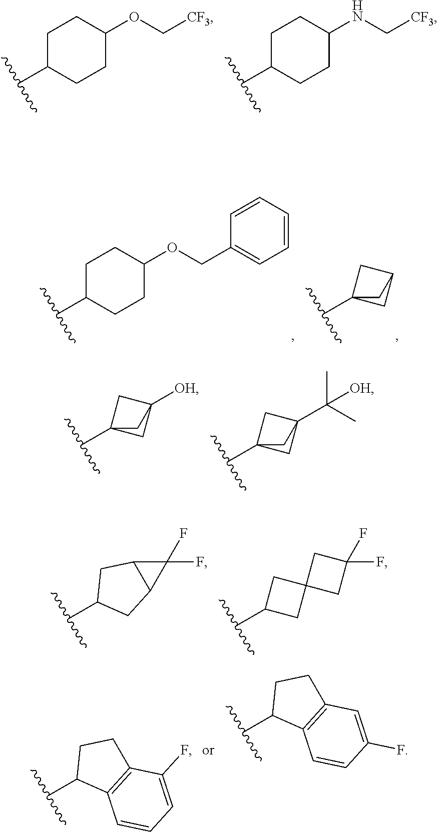

In some embodiments,

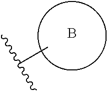

is saturated C₄₋₈cycloalkyl, wherein the C₄₋₈cycloalkyl is optionally substituted with one or more R^{a}, wherein each R^{a} is independently —OH, halo, C₁₋₆ alkyl, C₁₋₆haloalkyl, C₁₋₆ alkoxy, or —C(O)—C₁₋₆ alkoxy, wherein the C₁₋₆ alkoxy of R^{a} is optionally substituted with one or more C₁₋₆ alkoxy and the C₁₋₆ alkyl of R^{a} is optionally substituted with one or more —OH or C₁₋₆alkoxy. In some embodiments,

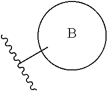

is

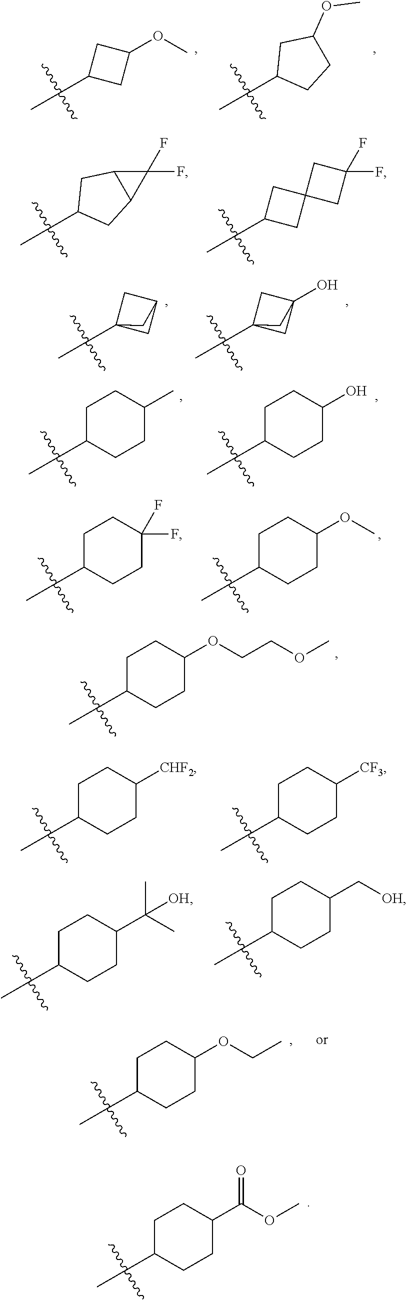

In some embodiments,

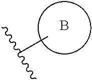

is

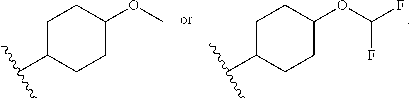

In some embodiments,

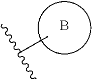

is

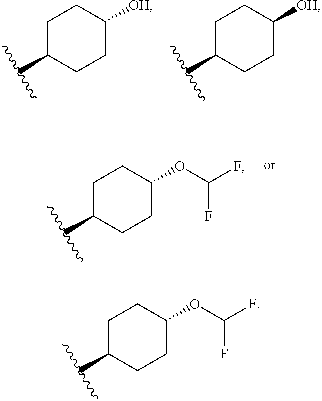 [SMILES: *[C@H]1CC[C@H](O)CC1 ; *[C@H]1CC[C@@H](O)CC1 ; *[C@H]1CC[C@H](OC(F)F)CC1 ; "or" ; *[C@H]1CC[C@H](OC(F)F)CC1]

In some embodiments,

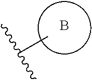

is

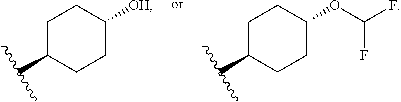 [SMILES: *[C@H]1CC[C@H](O)CC1 ; "or" ; *[C@H]1CC[C@H](OC(F)F)CC1]

In some embodiments,

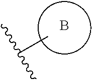

is

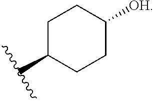 [SMILES: *[C@H]1CC[C@H](O)CC1]

In some embodiments,

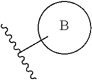

is

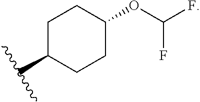 [SMILES: *[C@H]1CC[C@H](OC(F)F)CC1]

In some embodiments, provided herein is a compound of formula (I), such as a compound of formula (I-A1), (I-A2), (I-A3), (I-B1), (I-B2), (I-B3), (I-C), (I-D), (I-E), (I-F), (I-G), (I-H), or (I-J), or a stereoisomer or tautomer thereof, or a pharmaceutically acceptable salt of any of the foregoing, wherein

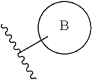

is saturated 4-8 membered heterocyclyl, wherein the 4-8 membered heterocyclyl is optionally substituted with one or more R^{b}, wherein each R^{b} is independently oxo, —C(O)—C₁₋₆alkyl, —C(O)—C₁₋₆alkoxy, or phenyl, wherein the phenyl of R^{b} is optionally substituted with one or more C₁₋₆ haloalkyl. In some embodiments,

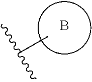

is

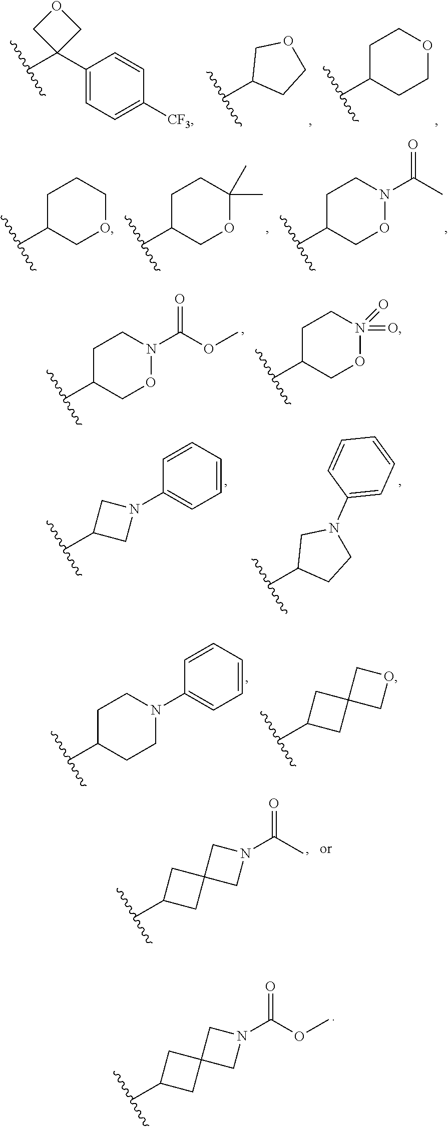

In some embodiments,

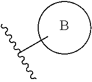

is

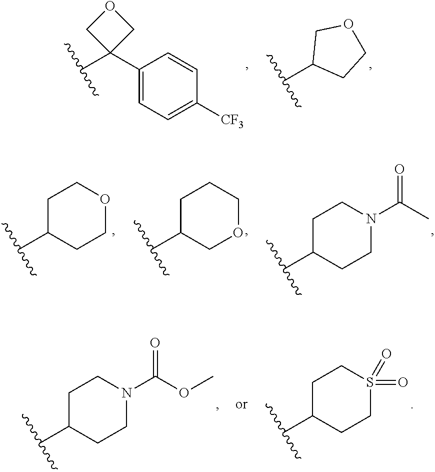

In some embodiments, provided herein is a compound of formula (I), such as a compound of formula (I-A1), (I-A2), (I-A3), (I-B1), (I-B2), (I-B3), (I-C), (I-D), (I-E), (I-F), (I-G), (I-H), or (I-J), or a stereoisomer or tautomer thereof, or a pharmaceutically acceptable salt of any of the foregoing, wherein

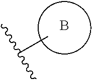

is

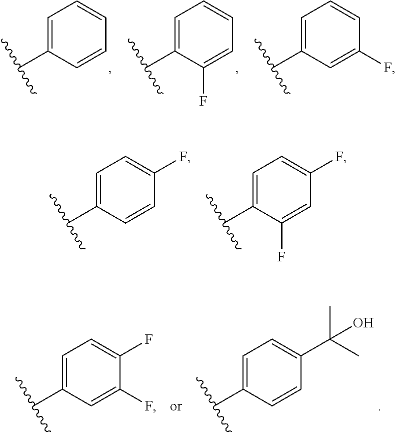

In some embodiments

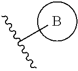

is phenyl, wherein the phenyl is optionally substituted with one or more halo. In some embodiments,

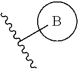

is phenyl, wherein the phenyl is optionally substituted with one or more fluoro. In some embodiments,

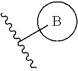

is phenyl, wherein the phenyl is optionally substituted with one or two fluoro. In some embodiments,

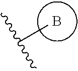

is

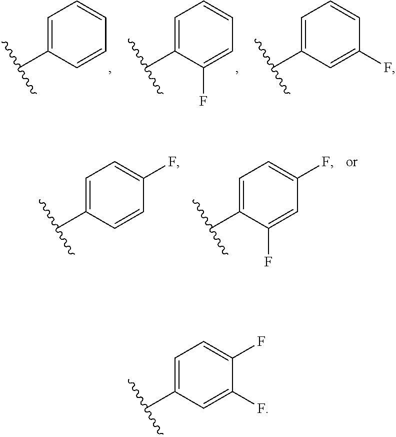

In some embodiments,

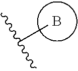

is phenyl substituted with C₁₋₆ alkyl optionally substituted with —OH. In some embodiments,

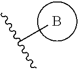

is

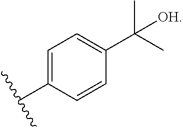 [SMILES: *c1ccc(C(C)(C)O)cc1]

In some embodiments, provided herein is a compound of formula (I), such as a compound of formula (I-A1), (I-A2), (I-A3), (I-B1), (I-B2), (I-B3), (I-C), (I-D), (I-E), (I-F), (I-G), (I-H), or (I-J), or a stereoisomer or tautomer thereof, or a pharmaceutically acceptable salt of any of the foregoing, wherein

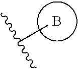

is pyridinyl, wherein the pyridinyl is optionally substituted with one or more halo, C₁₋₆ alkyl, C₁₋₆ haloalkyl, or C₁₋₆ alkoxy. In some embodiments,

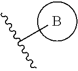

is

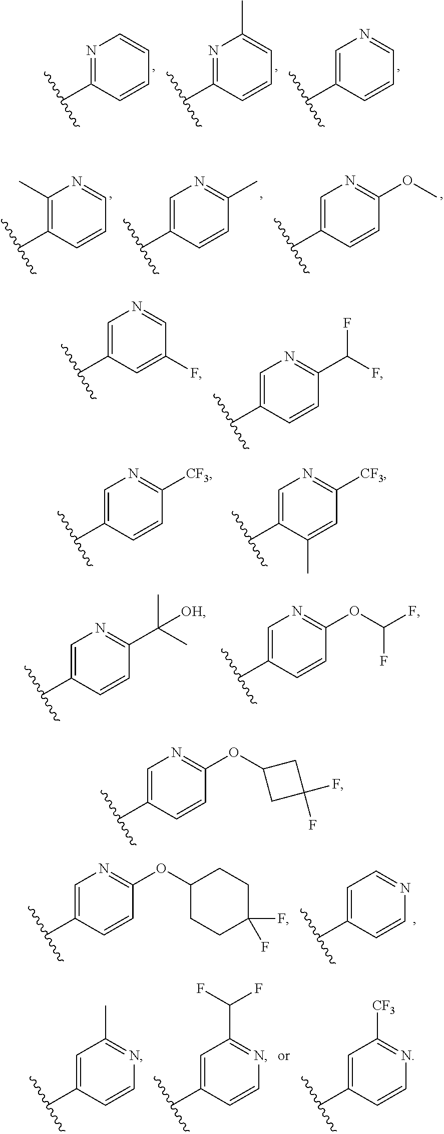

In some embodiments,

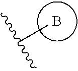

is

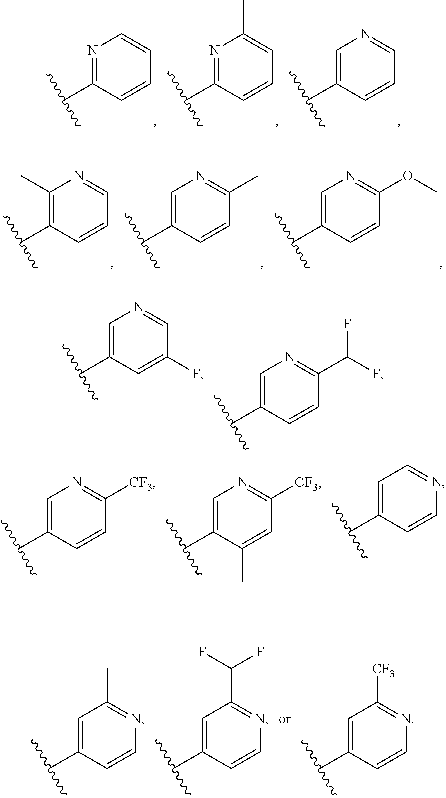

In some embodiments,

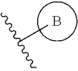

is

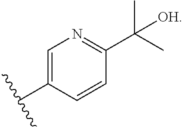 [SMILES: *c1ccc(C(C)(C)O)nc1]

In some embodiments, provided herein are compounds, and pharmaceutically acceptable salts thereof, described in Table 1.

TABLE 1

| Compound | | |
|---|---|---|
| Number | Structure | Name |
| 1 | 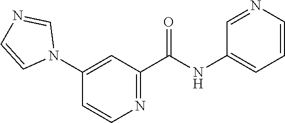 [SMILES: O=C(Nc1cccnc1)c1cc(-n2ccnc2)ccn1] | 4-(1H-imidazol-1-yl)-N-(pyridin-3- yl)picolinamide |
| 2 | 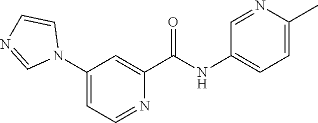 [SMILES: Cc1ccc(NC(=O)c2cc(-n3ccnc3)ccn2)cn1] | 4-(1H-imidazol-1-yl)-N-(6- methylpyridin-3-yl)picolinamide |
| 3 | 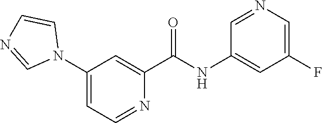 [SMILES: O=C(Nc1cncc(F)c1)c1cc(-n2ccnc2)ccn1] | N-(5-fluoropyridin-3-yl)-4-(1H- imidazol-1-yl)picolinamide |
| 4 | 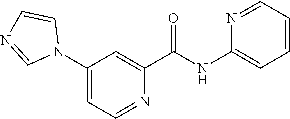 [SMILES: O=C(Nc1ccccn1)c1cc(-n2ccnc2)ccn1] | 4-(1H-imidazol-1-yl)-N-(pyridin-2- yl)picolinamide |
| 5 | 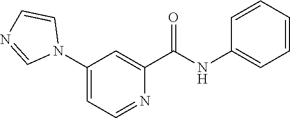 [SMILES: O=C(Nc1ccccc1)c1cc(-n2ccnc2)ccn1] | 4-(1H-imidazol-1-yl)-N- phenylpicolinamide |
| 6 | 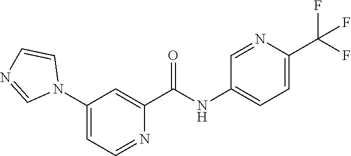 [SMILES: O=C(Nc1ccc(C(F)(F)F)nc1)c1cc(-n2ccnc2)ccn1] | 4-(1H-imidazol-1-yl)-N-(6- (trifluoromethyl)pyridin-3- yl)picolinamide |
| 7 | 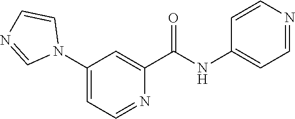 [SMILES: O=C(Nc1ccncc1)c1cc(-n2ccnc2)ccn1] | 4-(1H-imidazol-1-yl)-N-(pyridin-4- yl)picolinamide |
| 8 | 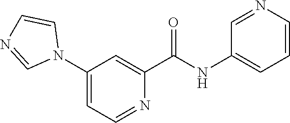 [SMILES: O=C(Nc1cccnc1)c1cc(-n2ccnc2)ccn1] | 6-(1H-imidazol-1-yl)-N-(pyridin-3- yl)picolinamide |
| 9 | 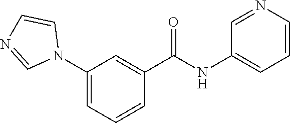 [SMILES: O=C(Nc1cccnc1)c1cccc(-n2ccnc2)c1] | 3-(1H-imidazol-1-yl)-N-(pyridin-3- yl)benzamide |
| 10 | 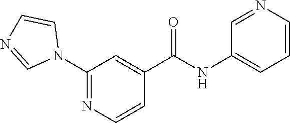 [SMILES: O=C(Nc1cccnc1)c1ccnc(-n2ccnc2)c1] | 2-(1H-imidazol-1-yl)-N-(pyridin-3- yl)isonicotinamide |
| 11 | 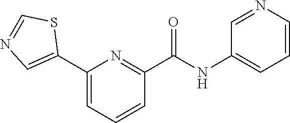 [SMILES: O=C(Nc1cccnc1)c1cccc(-c2cncs2)n1] | N-(pyridin-3-yl)-6-(thiazol-5- yl)picolinamide |
| 12 | 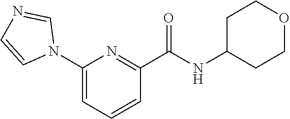 [SMILES: O=C(NC1CCOCC1)c1cccc(-n2ccnc2)n1] | 6-(1H-imidazol-1-yl)-N-(tetrahydro- 2H-pyran-4-yl)picolinamide |
| 13 | 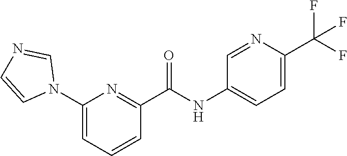 [SMILES: O=C(Nc1ccc(C(F)(F)F)nc1)c1cccc(-n2ccnc2)n1] | 6-(1H-imidazol-1-yl)-N-(6- (trifluoromethyl)pyridin-3- yl)picolinamide |
| 14 | 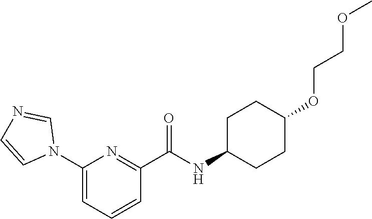 [SMILES: COCCO[C@H]1CC[C@H](NC(=O)c2cccc(-n3ccnc3)n2)CC1] | 6-(1H-imidazol-1-yl)-N-((1r,4r)-4-(2- methoxyethoxy)cyclohexyl)picolinamide |
| 15 | 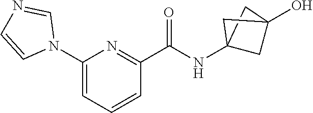 [SMILES: O=C(NC12CC(O)(C1)C2)c1cccc(-n2ccnc2)n1] | N-(3-hydroxybicyclo[1.1.1]pentan-1- yl)-6-(1H-imidazol-1-yl)picolinamide |
| 16 | 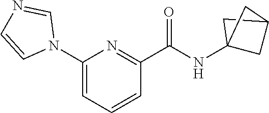 [SMILES: O=C(NC12CC(C1)C2)c1cccc(-n2ccnc2)n1] | N-(bicyclo[1.1.1]pentan-1-yl)-6-(1H- imidazol-1-yl)picolinamide |
| 17 | 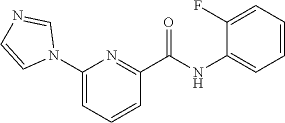 [SMILES: O=C(Nc1ccccc1F)c1cccc(-n2ccnc2)n1] | N-(2-fluorophenyl)-6-(1H-imidazol-1- yl)picolinamide |
| 18 | 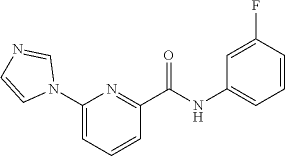 [SMILES: O=C(Nc1cccc(F)c1)c1cccc(-n2ccnc2)n1] | N-(3-fluorophenyl)-6-(1H-imidazol-1- yl)picolinamide |
| 19 | 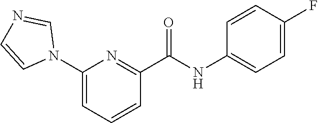 [SMILES: O=C(Nc1ccc(F)cc1)c1cccc(-n2ccnc2)n1] | N-(4-fluorophenyl)-6-(1H-imidazol-1- yl)picolinamide |
| 20 | 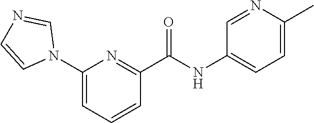 [SMILES: Cc1ccc(NC(=O)c2cccc(-n3ccnc3)n2)cn1] | 6-(1H-imidazol-1-yl)-N-(6- methylpyridin-3-yl)picolinamide |
| 21 | 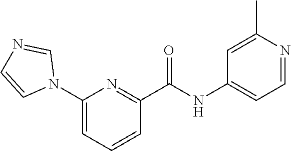 [SMILES: Cc1cc(NC(=O)c2cccc(-n3ccnc3)n2)ccn1] | 6-(1H-imidazol-1-yl)-N-(2- methylpyridin-4-yl)picolinamide |
| 22 | 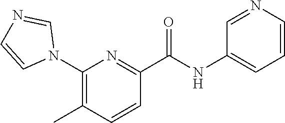 [SMILES: Cc1ccc(C(=O)Nc2cccnc2)nc1-n1ccnc1] | 6-(1H-imidazol-1-yl)-5-methyl-N- (pyridin-3-yl)picolinamide |
| 23 | 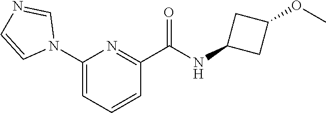 [SMILES: CO[C@H]1C[C@H](NC(=O)c2cccc(-n3ccnc3)n2)C1] | 6-(1H-imidazol-1-yl)-N-((1r,3r)-3- methoxycyclobutyl)picolinamide |
| 24 | 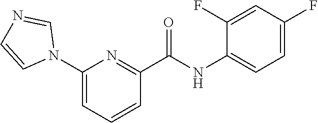 [SMILES: O=C(Nc1ccc(F)cc1F)c1cccc(-n2ccnc2)n1] | N-(2,4-difluorophenyl)-6-(1H- imidazol-1-yl)picolinamide |
| 25 | 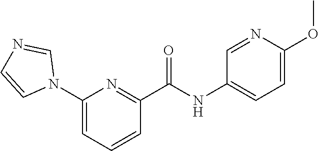 [SMILES: COc1ccc(NC(=O)c2cccc(-n3ccnc3)n2)cn1] | 6-(1H-imidazol-1-yl)-N-(6- methoxypyridin-3-yl)picolinamide |
| 26 | 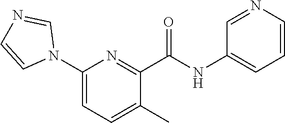 [SMILES: Cc1ccc(-n2ccnc2)nc1C(=O)Nc1cccnc1] | 6-(1H-imidazol-1-yl)-3-methyl-N- (pyridin-3-yl)picolinamide |
| 27 | 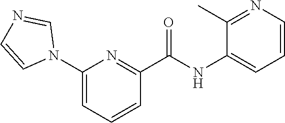 [SMILES: Cc1ncccc1NC(=O)c1cccc(-n2ccnc2)n1] | 6-(1H-imidazol-1-yl)-N-(2- methylpyridin-3-yl)picolinamide |
| 28 | 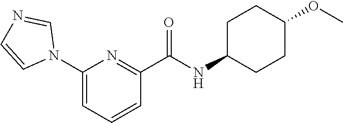 [SMILES: CO[C@H]1CC[C@H](NC(=O)c2cccc(-n3ccnc3)n2)CC1] | 6-(1H-imidazol-1-yl)-N-((1r,4r)-4- methoxycyclohexyl)picolinamide |
| 29 | 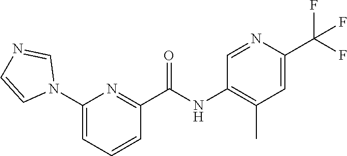 [SMILES: Cc1cc(C(F)(F)F)ncc1NC(=O)c1cccc(-n2ccnc2)n1] | 6-(1H-imidazol-1-yl)-N-(4-methyl-6- (trifluoromethyl)pyridin-3- yl)picolinamide |
| 30 | 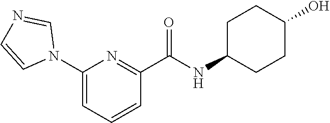 [SMILES: O=C(N[C@H]1CC[C@H](O)CC1)c1cccc(-n2ccnc2)n1] | N-((1r,4r)-4-hydroxycyclohexyl)-6- (1H-imidazol-1-yl)picolinamide |
| 31 | 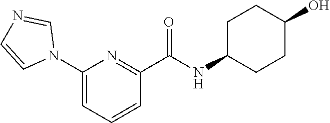 [SMILES: O=C(N[C@H]1CC[C@@H](O)CC1)c1cccc(-n2ccnc2)n1] | N-((1s,4s)-4-hydroxycyclohexyl)-6- (1H-imidazol-1-yl)picolinamide |
| 32 | 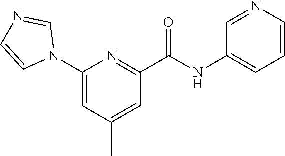 [SMILES: Cc1cc(C(=O)Nc2cccnc2)nc(-n2ccnc2)c1] | 6-(1H-imidazol-1-yl)-4-methyl-N- (pyridin-3-yl)picolinamide |
| 33 | 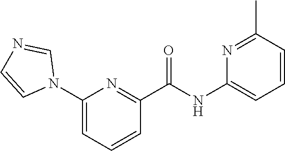 [SMILES: Cc1cccc(NC(=O)c2cccc(-n3ccnc3)n2)n1] | 6-(1H-imidazol-1-yl)-N-(6- methylpyridin-2-yl)picolinamide |
| 34 | 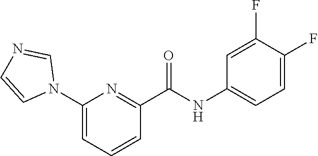 [SMILES: O=C(Nc1ccc(F)c(F)c1)c1cccc(-n2ccnc2)n1] | N-(3,4-difluorophenyl)-6-(1H- imidazol-1-yl)picolinamide |
| 35 | 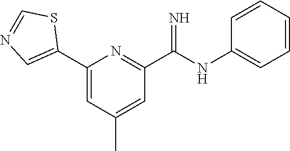 [SMILES: Cc1cc(C(=N)Nc2ccccc2)nc(-c2cncs2)c1] | 4-methyl-N-(pyridin-3-yl)-6-(thiazol- 5-yl)picolinamide |
| 36 | 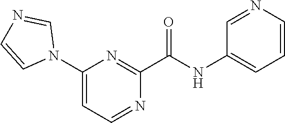 [SMILES: O=C(Nc1cccnc1)c1nccc(-n2ccnc2)n1] | 4-(1H-imidazol-1-yl)-N-(pyridin-3- yl)pyrimidine-2-carboxamide |
| 37 | 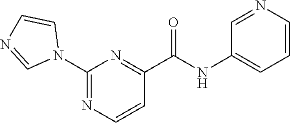 [SMILES: O=C(Nc1cccnc1)c1ccnc(-n2ccnc2)n1] | 2-(1H-imidazol-1-yl)-N-(pyridin-3- yl)pyrimidine-4-carboxamide |
| 38 | 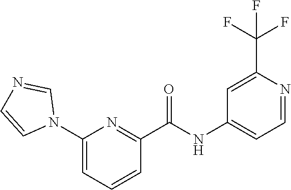 [SMILES: O=C(Nc1ccnc(C(F)(F)F)c1)c1cccc(-n2ccnc2)n1] | 6-(1H-imidazol-1-yl)-N-(2- (trifluoromethyl)pyridin-4- yl)picolinamide |
| 39 | 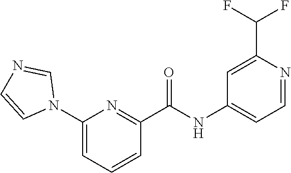 [SMILES: O=C(Nc1ccnc(C(F)F)c1)c1cccc(-n2ccnc2)n1] | N-(2-(difluoromethyl)pyridin-4-yl)-6- (1H-imidazol-1-yl)picolinamide |
| 40 | 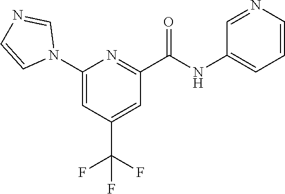 [SMILES: O=C(Nc1cccnc1)c1cc(C(F)(F)F)cc(-n2ccnc2)n1] | 6-(1H-imidazol-1-yl)-N-(pyridin-3- yl)-4-(trifluoromethyl)picolinamide |
| 41 | 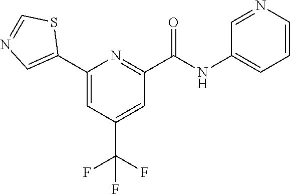 [SMILES: O=C(Nc1cccnc1)c1cc(C(F)(F)F)cc(-c2cncs2)n1] | N-(pyridin-3-yl)-6-(thiazol-5-yl)-4- (trifluoromethyl)picolinamide |
| 42 | 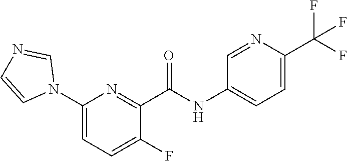 [SMILES: O=C(Nc1ccc(C(F)(F)F)nc1)c1nc(-n2ccnc2)ccc1F] | 3-fluoro-6-(1H-imidazol-1-yl)-N-(6- (trifluoromethyl)pyridin-3- yl)picolinamide |
| 43 | 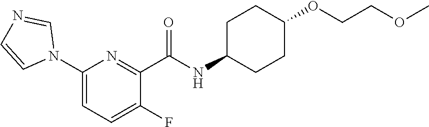 [SMILES: COCCO[C@H]1CC[C@H](NC(=O)c2nc(-n3ccnc3)ccc2F)CC1] | 3-fluoro-6-(1H-imidazol-1-yl)-N- ((1r,4r)-4-(2- methoxyethoxy)cyclohexyl)picolinamide |
| 44 | 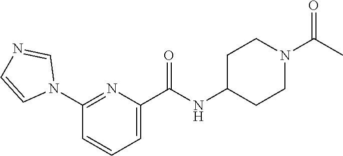 [SMILES: CC(=O)N1CCC(NC(=O)c2cccc(-n3ccnc3)n2)CC1] | N-(1-acetylpiperidin-4-yl)-6-(1H- imidazol-1-yl)picolinamide |
| 45 | 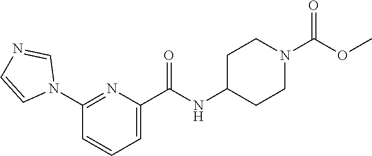 [SMILES: COC(=O)N1CCC(NC(=O)c2cccc(-n3ccnc3)n2)CC1] | methyl 4-(6-(1H-imidazol-1- yl)picolinamido)piperidine-1- carboxylate |
| 46 | 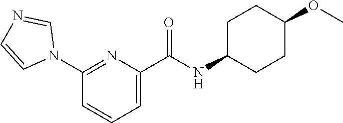 [SMILES: CO[C@H]1CC[C@@H](NC(=O)c2cccc(-n3ccnc3)n2)CC1] | 6-(1H-imidazol-1-yl)-N-((1s,4s)-4- methoxycyclohexyl)picolinamide |
| 47 | 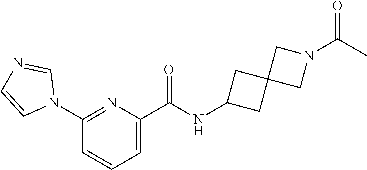 [SMILES: CC(=O)N1CC2(CC(NC(=O)c3cccc(-n4ccnc4)n3)C2)C1] | N-(2-acetyl-2-azaspiro[3.3]heptan-6- yl)-6-(1H-imidazol-1-yl)picolinamide |
| 48 | 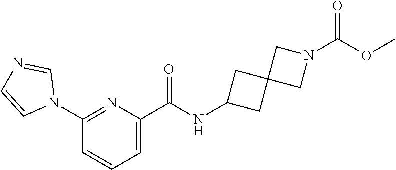 [SMILES: COC(=O)N1CC2(CC(NC(=O)c3cccc(-n4ccnc4)n3)C2)C1] | methyl 6-(6-(1H-imidazol-1- yl)picolinamido)-2- azaspiro[3.3]heptane-2-carboxylate |
| 49 | 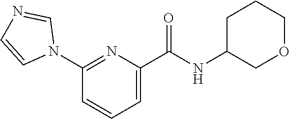 [SMILES: O=C(NC1CCCOC1)c1cccc(-n2ccnc2)n1] | 6-(1H-imidazol-1-yl)-N-(tetrahydro- 2H-pyran-3-yl)picolinamide |
| 50 | 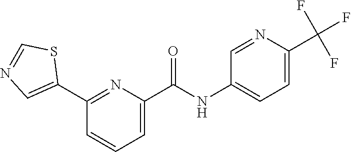 [SMILES: O=C(Nc1ccc(C(F)(F)F)nc1)c1cccc(-c2cncs2)n1] | 6-(thiazol-5-yl)-N-(6- (trifluoromethyl)pyridin-3- yl)picolinamide |
| 51 | 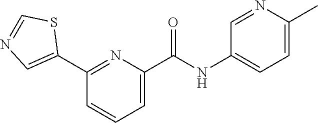 [SMILES: Cc1ccc(NC(=O)c2cccc(-c3cncs3)n2)cn1] | N-(6-methylpyridin-3-yl)-6-(thiazol- 5-yl)picolinamide |
| 52 | 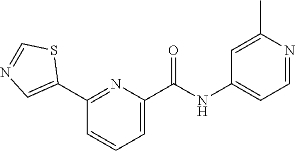 [SMILES: Cc1cc(NC(=O)c2cccc(-c3cncs3)n2)ccn1] | N-(2-methylpyridin-4-yl)-6-(thiazol- 5-yl)picolinamide |
| 53 | 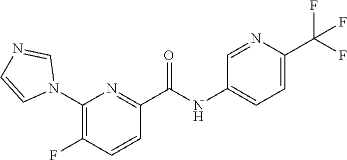 [SMILES: O=C(Nc1ccc(C(F)(F)F)nc1)c1ccc(F)c(-n2ccnc2)n1] | 5-fluoro-6-(1H-imidazol-1-yl)-N-(6- (trifluoromethyl)pyridin-3- yl)picolinamide |
| 54 | 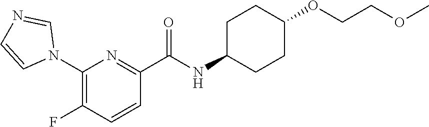 [SMILES: COCCO[C@H]1CC[C@H](NC(=O)c2ccc(F)c(-n3ccnc3)n2)CC1] | 5-fluoro-6-(1H-imidazol-1-yl)-N- ((1r,4r)-4-(2- methoxyethoxy)cyclohexyl)picolinamide |
| 55 | 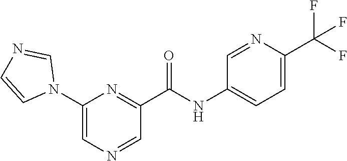 [SMILES: O=C(Nc1ccc(C(F)(F)F)nc1)c1cncc(-n2ccnc2)n1] | 6-(1H-imidazol-1-yl)-N-(6- (trifluoromethyl)pyridin-3- yl)pyrazine-2-carboxamide |
| 56 | 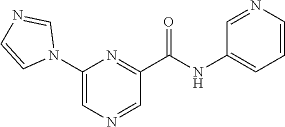 [SMILES: O=C(Nc1cccnc1)c1cncc(-n2ccnc2)n1] | 6-(1H-imidazol-1-yl)-N-(pyridin-3- yl)pyrazine-2-carboxamide |
| 57 | 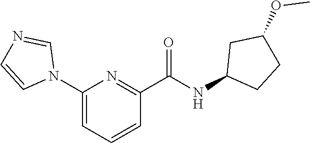 [SMILES: CO[C@@H]1CC[C@@H](NC(=O)c2cccc(-n3ccnc3)n2)C1] | 6-(1H-imidazol-1-yl)-N-((1R,3R)-3- methoxycyclopentyl)picolinamide |
| 58 | 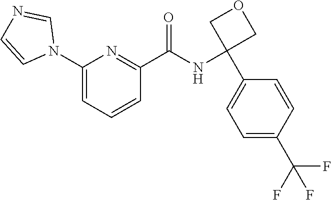 [SMILES: O=C(NC1(c2ccc(C(F)(F)F)cc2)COC1)c1cccc(-n2ccnc2)n1] | 6-(1H-imidazol-1-yl)-N-(3-(4- (trifluoromethyl)phenyl)oxetan-3- yl)picolinamide |
| 59 | 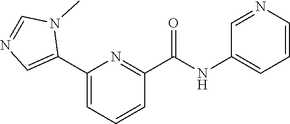 [SMILES: Cn1cncc1-c1cccc(C(=O)Nc2cccnc2)n1] | 6-(1-methyl-1H-imidazol-5-yl)-N- (pyridin-3-yl)picolinamide |
| 60 | 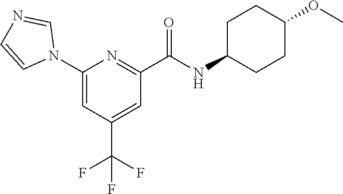 [SMILES: CO[C@H]1CC[C@H](NC(=O)c2cc(C(F)(F)F)cc(-n3ccnc3)n2)CC1] | 6-(1H-imidazol-1-yl)-N-((1r,4r)-4- methoxycyclohexyl)-4- (trifluoromethyl)picolinamide |
| 61 | 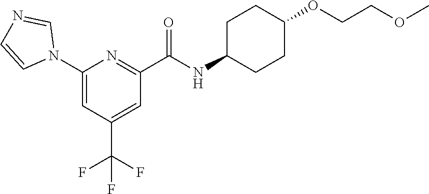 [SMILES: COCCO[C@H]1CC[C@H](NC(=O)c2cc(C(F)(F)F)cc(-n3ccnc3)n2)CC1] | 6-(1H-imidazol-1-yl)-N-((1r,4r)-4-(2- methoxyethoxy)cyclohexyl)-4- (trifluoromethyl)picolinamide |
| 62 | 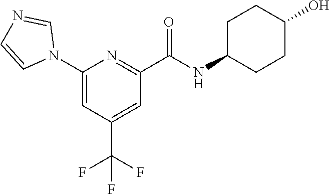 [SMILES: O=C(N[C@H]1CC[C@H](O)CC1)c1cc(C(F)(F)F)cc(-n2ccnc2)n1] | N-((1r,4r)-4-hydroxycyclohexyl)-6- (1H-imidazol-1-yl)-4- (trifluoromethyl)picolinamide |
| 63 | 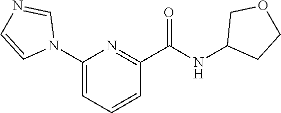 [SMILES: O=C(NC1CCOC1)c1cccc(-n2ccnc2)n1] | 6-(1H-imidazol-1-yl)-N- (tetrahydrofuran-3-yl)picolinamide |
| 64 | 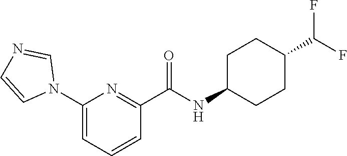 [SMILES: O=C(N[C@H]1CC[C@H](C(F)F)CC1)c1cccc(-n2ccnc2)n1] | N-((1r,4r)-4- (difluoromethyl)cyclohexyl)-6-(1H- imidazol-1-yl)picolinamide |
| 65 | 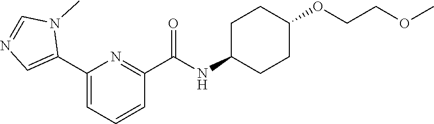 [SMILES: COCCO[C@H]1CC[C@H](NC(=O)c2cccc(-c3cncn3C)n2)CC1] | N-((1r,4r)-4-(2- methoxyethoxy)cyclohexyl)-6-(1- methyl-1H-imidazol-5- yl)picolinamide |
| 66 | 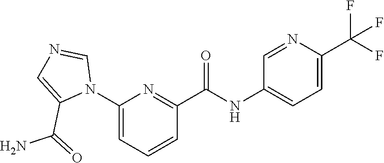 [SMILES: NC(=O)c1cncn1-c1cccc(C(=O)Nc2ccc(C(F)(F)F)nc2)n1] | 6-(5-carbamoyl-1H-imidazol-1-yl)-N- (6-(trifluoromethyl)pyridin-3- yl)picolinamide |
| 67 | 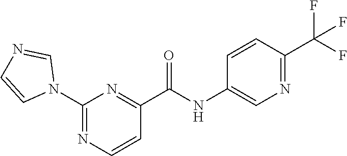 [SMILES: O=C(Nc1ccc(C(F)(F)F)nc1)c1ccnc(-n2ccnc2)n1] | 2-(1H-imidazol-1-yl)-N-(6- (trifluoromethyl)pyridin-3- yl)pyrimidine-4-carboxamide |
| 68 | 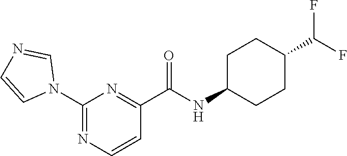 [SMILES: O=C(N[C@H]1CC[C@H](C(F)F)CC1)c1ccnc(-n2ccnc2)n1] | N-((1r,4r)-4- (difluoromethyl)cyclohexyl)-2-(1H- imidazol-1-yl)pyrimidine-4- carboxamide |
| 69 | 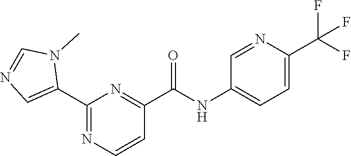 [SMILES: Cn1cncc1-c1nccc(C(=O)Nc2ccc(C(F)(F)F)nc2)n1] | 2-(1-methyl-1H-imidazol-5-yl)-N-(6- (trifluoromethyl)pyridin-3- yl)pyrimidine-4-carboxamide |
| 70 | 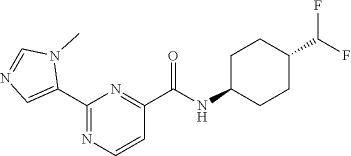 [SMILES: Cn1cncc1-c1nccc(C(=O)N[C@H]2CC[C@H](C(F)F)CC2)n1] | N-((1r,4r)-4- (difluoromethyl)cyclohexyl)-2-(1- methyl-1H-imidazol-5-yl)pyrimidine- 4-carboxamide |
| 71 | 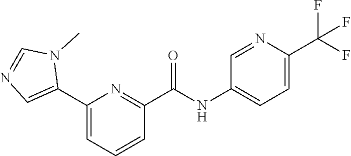 [SMILES: Cn1cncc1-c1cccc(C(=O)Nc2ccc(C(F)(F)F)nc2)n1] | 6-(1-methyl-1H-imidazol-5-yl)-N-(6- (trifluoromethyl)pyridin-3- yl)picolinamide |
| 72 | 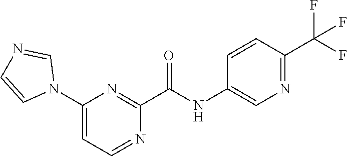 [SMILES: O=C(Nc1ccc(C(F)(F)F)nc1)c1nccc(-n2ccnc2)n1] | 4-(1H-imidazol-1-yl)-N-(6- (trifluoromethyl)pyridin-3- yl)pyrimidine-2-carboxamide |
| 73 | 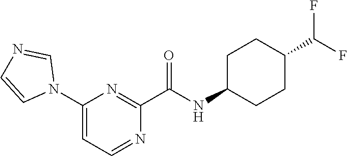 [SMILES: O=C(N[C@H]1CC[C@H](C(F)F)CC1)c1nccc(-n2ccnc2)n1] | N-((1r,4r)-4- (difluoromethyl)cyclohexyl)-4-(1H- imidazol-1-yl)pyrimidine-2- carboxamide |
| 74 | 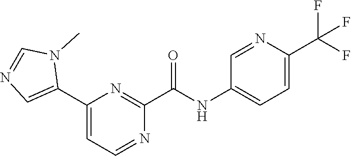 [SMILES: Cn1cncc1-c1ccnc(C(=O)Nc2ccc(C(F)(F)F)nc2)n1] | 4-(1-methyl-1H-imidazol-5-yl)-N-(6- (trifluoromethyl)pyridin-3- yl)pyrimidine-2-carboxamide |
| 75 | 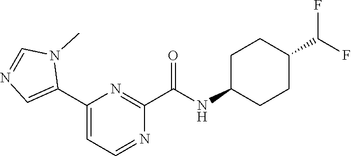 [SMILES: Cn1cncc1-c1ccnc(C(=O)N[C@H]2CC[C@H](C(F)F)CC2)n1] | N-((1r,4r)-4- (difluoromethyl)cyclohexyl)-4-(1- methyl-1H-imidazol-5-yl)pyrimidine- 2-carboxamide |
| 76 | 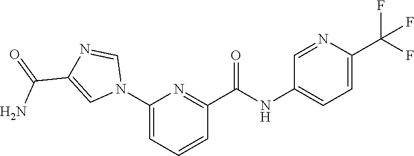 [SMILES: NC(=O)c1cn(-c2cccc(C(=O)Nc3ccc(C(F)(F)F)nc3)n2)cn1] | 6-(4-carbamoyl-1H-imidazol-1-yl)-N- (6-(trifluoromethyl)pyridin-3- yl)picolinamide |
| 77 | 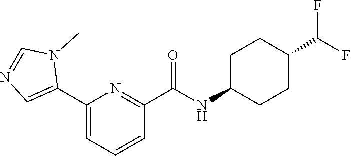 [SMILES: Cn1cncc1-c1cccc(C(=O)N[C@H]2CC[C@H](C(F)F)CC2)n1] | N-((1r,4r)-4- (difluoromethyl)cyclohexyl)-6-( 1- methyl-1H-imidazol-5- yl)picolinamide |
| 78 | 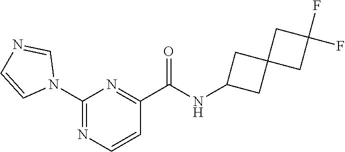 [SMILES: O=C(NC1CC2(C1)CC(F)(F)C2)c1ccnc(-n2ccnc2)n1] | N-(6,6-difluorospiro[3.3]heptan-2-yl)- 2-(1H-imidazol-1-yl)pyrimidine-4- carboxamide |
| 79 | 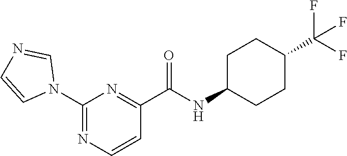 [SMILES: O=C(N[C@H]1CC[C@H](C(F)(F)F)CC1)c1ccnc(-n2ccnc2)n1] | 2-(1H-imidazol-1-yl)-N-((1r,4r)-4- (trifluoromethyl)cyclohexyl)pyrimidine- 4-carboxamide |
| 80 | 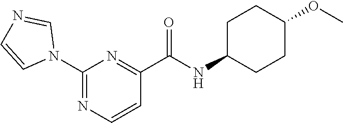 [SMILES: CO[C@H]1CC[C@H](NC(=O)c2ccnc(-n3ccnc3)n2)CC1] | 2-(1H-imidazol-1-yl)-N-((1r,4r)-4- methoxycyclohexyl)pyrimidine-4- carboxamide |
| 81 | 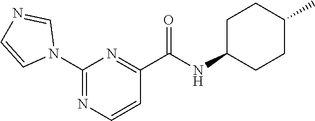 [SMILES: C[C@H]1CC[C@H](NC(=O)c2ccnc(-n3ccnc3)n2)CC1] | 2-(1H-imidazol-1-yl)-N-((1r,4r)-4- methylcyclohexyl)pyrimidine-4- carboxamide |
| 82 | 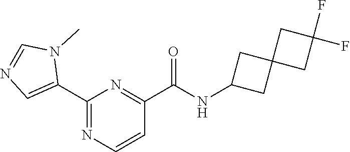 [SMILES: Cn1cncc1-c1nccc(C(=O)NC2CC3(C2)CC(F)(F)C3)n1] | N-(6,6-difluorospiro[3.3]heptan-2-yl)- 2-(1-methyl-1H-imidazol-5- yl)pyrimidine-4-carboxamide |
| 83 | 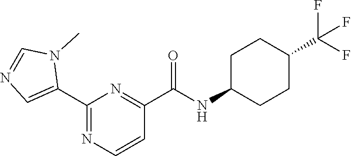 [SMILES: Cn1cncc1-c1nccc(C(=O)N[C@H]2CC[C@H](C(F)(F)F)CC2)n1] | 2-(1-methyl-1H-imidazol-5-yl)-N- ((1r,4r)-4- (trifluoromethyl)cyclohexyl)pyrimidine- 4-carboxamide |
| 84 | 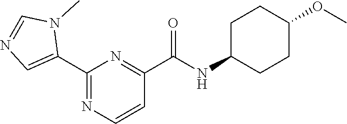 [SMILES: CO[C@H]1CC[C@H](NC(=O)c2ccnc(-c3cncn3C)n2)CC1] | N-((1r,4r)-4-methoxycyclohexyl)-2- (1-methyl-1H-imidazol-5- yl)pyrimidine-4-carboxamide |
| 85 | 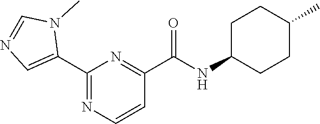 [SMILES: Cn1cncc1-c1nccc(C(=O)N[C@H]2CC[C@H](C)CC2)n1] | 2-(1-methyl-1H-imidazol-5-yl)-N- ((1r,4r)-4- methylcyclohexyl)pyrimidine-4- carboxamide |
| 86 | 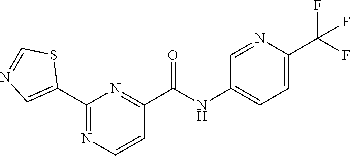 [SMILES: O=C(Nc1ccc(C(F)(F)F)nc1)c1ccnc(-c2cncs2)n1] | 2-(thiazol-5-yl)-N-(6- (trifluoromethyl)pyridin-3- yl)pyrimidine-4-carboxamide |
| 87 | 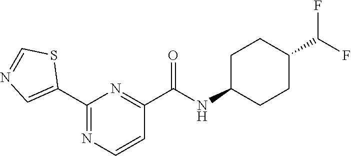 [SMILES: O=C(N[C@H]1CC[C@H](C(F)F)CC1)c1ccnc(-c2cncs2)n1] | N-((1r,4r)-4- (difluoromethyl)cyclohexyl)-2- (thiazol-5-yl)pyrimidine-4- carboxamide |
| 88 | 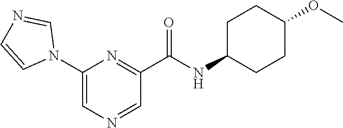 [SMILES: CO[C@H]1CC[C@H](NC(=O)c2cncc(-n3ccnc3)n2)CC1] | 6-(1H-imidazol-1-yl)-N-((1r,4r)-4- methoxycyclohexyl)pyrazine-2- carboxamide |
| 89 | 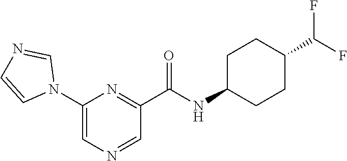 [SMILES: O=C(N[C@H]1CC[C@H](C(F)F)CC1)c1cncc(-n2ccnc2)n1] | N-((1r,4r)-4- (difluoromethyl)cyclohexyl)-6-(1H- imidazol-1-yl)pyrazine-2- carboxamide |
| 90 | 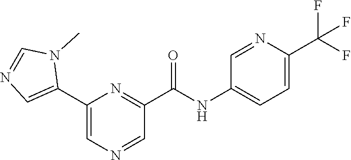 [SMILES: Cn1cncc1-c1cncc(C(=O)Nc2ccc(C(F)(F)F)nc2)n1] | 6-(1-methyl-1H-imidazol-5-yl)-N-(6- (trifluoromethyl)pyridin-3- yl)pyrazine-2-carboxamide |
| 91 | 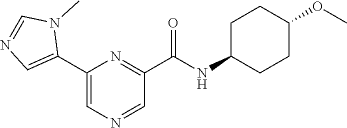 [SMILES: CO[C@H]1CC[C@H](NC(=O)c2cncc(-c3cncn3C)n2)CC1] | N-((1r,4r)-4-methoxycyclohexyl)-6- (1-methyl-1H-imidazol-5-yl)pyrazine- 2-carboxamide |
| 92 | 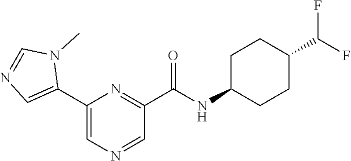 [SMILES: Cn1cncc1-c1cncc(C(=O)N[C@H]2CC[C@H](C(F)F)CC2)n1] | N-((1r,4r)-4- (difluoromethyl)cyclohexyl)-6-( 1- methyl-1H-imidazol-5-yl)pyrazine-2- carboxamide |
| 93 | 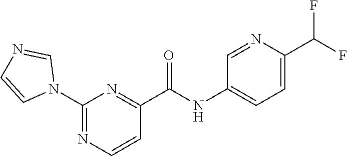 [SMILES: O=C(Nc1ccc(C(F)F)nc1)c1ccnc(-n2ccnc2)n1] | N-(6-(difluoromethyl)pyridin-3-yl)-2- (1H-imidazol-1-yl)pyrimidine-4- carboxamide |
| 94 | 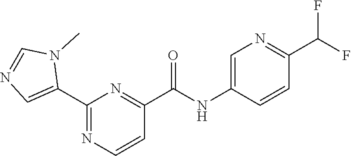 [SMILES: Cn1cncc1-c1nccc(C(=O)Nc2ccc(C(F)F)nc2)n1] | N-(6-(difluoromethyl)pyridin-3-yl)-2- (1-methyl-1H-imidazol-5- yl)pyrimidine-4-carboxamide |
| 95 | 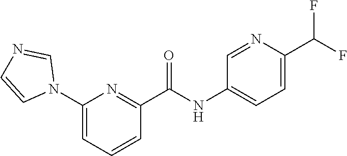 [SMILES: O=C(Nc1ccc(C(F)F)nc1)c1cccc(-n2ccnc2)n1] | N-(6-(difluoromethyl)pyridin-3-yl)-6- (1H-imidazol-1-yl)picolinamide |
| 96 | 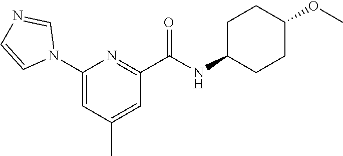 [SMILES: CO[C@H]1CC[C@H](NC(=O)c2cc(C)cc(-n3ccnc3)n2)CC1] | 6-(1H-imidazol-1-yl)-N-((1r,4r)-4- methoxycyclohexyl)-4- methylpicolinamide |
| 97 | 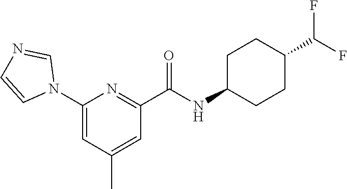 [SMILES: Cc1cc(C(=O)N[C@H]2CC[C@H](C(F)F)CC2)nc(-n2ccnc2)c1] | N-((1r,4r)-4- (difluoromethyl)cyclohexyl)-6-(1H- imidazol-1-yl)-4-methylpicolinamide |
| 98 | 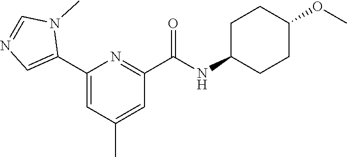 [SMILES: CO[C@H]1CC[C@H](NC(=O)c2cc(C)cc(-c3cncn3C)n2)CC1] | N-((1r,4r)-4-methoxycyclohexyl)-4- methyl-6-(1-methyl-1H-imidazol-5- yl)picolinamide |
| 99 | 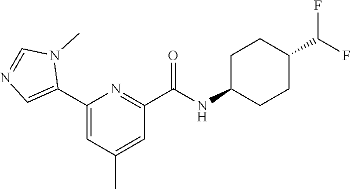 [SMILES: Cc1cc(C(=O)N[C@H]2CC[C@H](C(F)F)CC2)nc(-c2cncn2C)c1] | N-((1r,4r)-4- (difluoromethyl)cyclohexyl)-4- methyl-6-(1-methyl-1H-imidazol-5- yl)picolinamide |
| 100 | 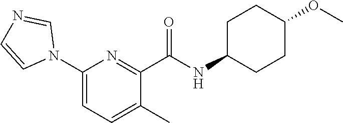 [SMILES: CO[C@H]1CC[C@H](NC(=O)c2nc(-n3ccnc3)ccc2C)CC1] | 6-(1H-imidazol-1-yl)-N-((1r,4r)-4- methoxycyclohexyl)-3- methylpicolinamide |
| 101 | 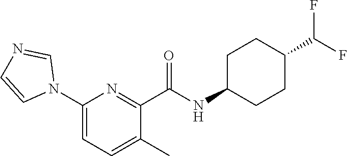 [SMILES: Cc1ccc(-n2ccnc2)nc1C(=O)N[C@H]1CC[C@H](C(F)F)CC1] | N-((1r,4r)-4- (difluoromethyl)cyclohexyl)-6-(1H- imidazol-1-yl)-3-methylpicolinamide |
| 102 | 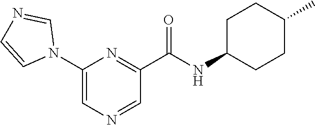 [SMILES: C[C@H]1CC[C@H](NC(=O)c2cncc(-n3ccnc3)n2)CC1] | 6-(1H-imidazol-1-yl)-N-((1r,4r)-4- methylcyclohexyl)pyrazine-2- carboxamide |
| 103 | 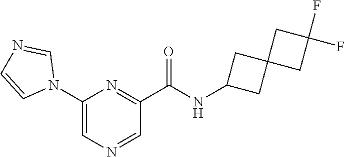 [SMILES: O=C(NC1CC2(C1)CC(F)(F)C2)c1cncc(-n2ccnc2)n1] | N-(6,6-difluorospiro[3.3]heptan-2-yl)- 6-(1H-imidazol-1-yl)pyrazine-2- carboxamide |
| 104 | 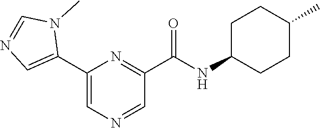 [SMILES: Cn1cncc1-c1cncc(C(=O)N[C@H]2CC[C@H](C)CC2)n1] | 6-(1-methyl-1H-imidazol-5-yl)-N- ((1r,4r)-4-methylcyclohexyl)pyrazine- 2-carboxamide |
| 105 | 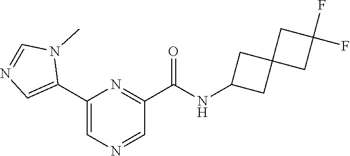 [SMILES: Cn1cncc1-c1cncc(C(=O)NC2CC3(C2)CC(F)(F)C3)n1] | N-(6,6-difluorospiro[3.3]heptan-2-yl)- 6-(1-methyl-1H-imidazol-5- yl)pyrazine-2-carboxamide |
| 106 | 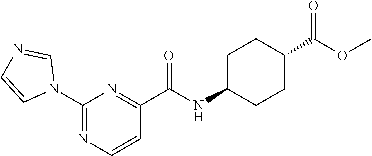 [SMILES: COC(=O)[C@H]1CC[C@H](NC(=O)c2ccnc(-n3ccnc3)n2)CC1] | methyl (1r,4r)-4-(2-(1H-imidazol-1- yl)pyrimidine-4- carboxamido)cyclohexane-1- carboxylate |
| 107 | 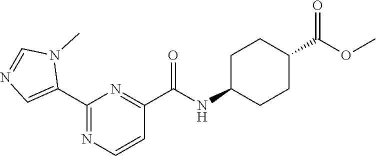 [SMILES: COC(=O)[C@H]1CC[C@H](NC(=O)c2ccnc(-c3cncn3C)n2)CC1] | methyl (1r,4r)-4-(2-(1-methyl-1H- imidazol-5-yl)pyrimidine-4- carboxamido)cyclohexane-1- carboxylate |
| 108 | 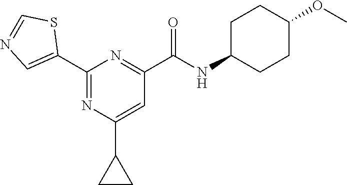 [SMILES: CO[C@H]1CC[C@H](NC(=O)c2cc(C3CC3)nc(-c3cncs3)n2)CC1] | 6-cyclopropyl-N-((1r,4r)-4- methoxycyclohexyl)-2-(thiazol-5- yl)pyrimidine-4-carboxamide |
| 109 | 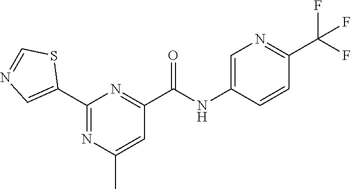 [SMILES: Cc1cc(C(=O)Nc2ccc(C(F)(F)F)nc2)nc(-c2cncs2)n1] | 6-cyclopropyl-2-(thiazol-5-yl)-N-(6- (trifluoromethyl)pyridin-3- yl)pyrimidine-4-carboxamide |
| 110 | 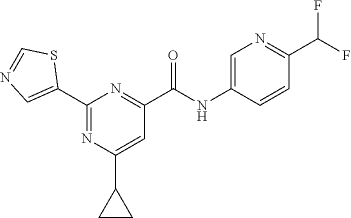 [SMILES: O=C(Nc1ccc(C(F)F)nc1)c1cc(C2CC2)nc(-c2cncs2)n1] | 6-cyclopropyl-N-(6- (difluoromethyl)pyridin-3-yl)-2- (thiazol-5-yl)pyrimidine-4- carboxamide |
| 111 | 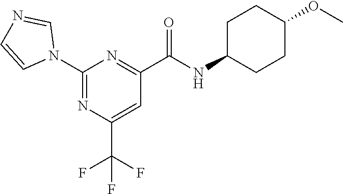 [SMILES: CO[C@H]1CC[C@H](NC(=O)c2cc(C(F)(F)F)nc(-n3ccnc3)n2)CC1] | 2-(1H-imidazol-1-yl)-N-((1r,4r)-4- methoxycyclohexyl)-6- (trifluoromethyl)pyrimidine-4-carboxamide |
| 112 | 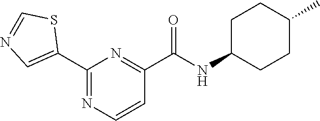 [SMILES: C[C@H]1CC[C@H](NC(=O)c2ccnc(-c3cncs3)n2)CC1] | N-((1r,4r)-4-methylcyclohexyl)-2- (thiazol-5-yl)pyrimidine-4- carboxamide |
| 113 | 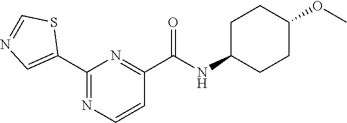 [SMILES: CO[C@H]1CC[C@H](NC(=O)c2ccnc(-c3cncs3)n2)CC1] | N-((1r,4r)-4-methoxycyclohexyl)-2- (thiazol-5-yl)pyrimidine-4- carboxamide |
| 114 | 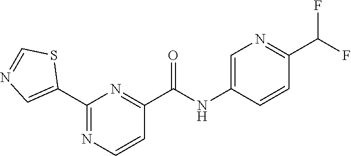 [SMILES: O=C(Nc1ccc(C(F)F)nc1)c1ccnc(-c2cncs2)n1] | N-(6-(difluoromethyl)pyridin-3-yl)-2- (thiazol-5-yl)pyrimidine-4- carboxamide |
| 115 | 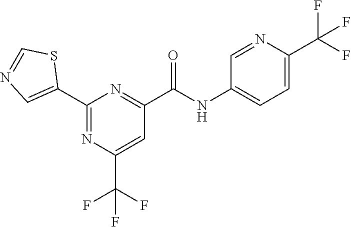 [SMILES: O=C(Nc1ccc(C(F)(F)F)nc1)c1cc(C(F)(F)F)nc(-c2cncs2)n1] | 2-(thiazol-5-yl)-6-(trifluoromethyl)- N-(6-(trifluoromethyl)pyridin-3- yl)pyrimidine-4-carboxamide |
| 116 | 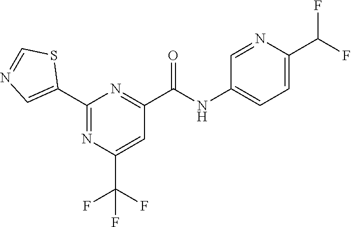 [SMILES: O=C(Nc1ccc(C(F)F)nc1)c1cc(C(F)(F)F)nc(-c2cncs2)n1] | N-(6-(difluoromethyl)pyridin-3-yl)-2- (thiazol-5-yl)-6- (trifluoromethyl)pyrimidine-4- carboxamide |
| 117 | 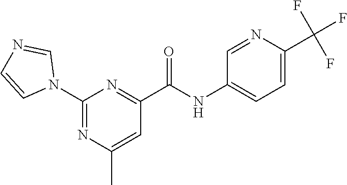 [SMILES: Cc1cc(C(=O)Nc2ccc(C(F)(F)F)nc2)nc(-n2ccnc2)n1] | 2-(1H-imidazol-1-yl)-6-methyl-N-(6- (trifluoromethyl)pyridin-3- yl)pyrimidine-4-carboxamide |
| 118 | 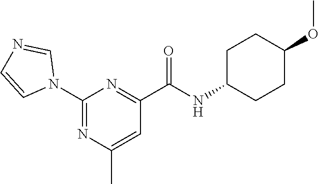 [SMILES: CO[C@H]1CC[C@H](NC(=O)c2cc(C)nc(-n3ccnc3)n2)CC1] | 2-(1H-imidazol-1-yl)-N-((1r,4r)-4- methoxycyclohexyl)-6- methylpyrimidine-4-carboxamide |
| 119 | 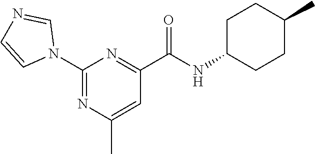 [SMILES: Cc1cc(C(=O)N[C@H]2CC[C@H](C)CC2)nc(-n2ccnc2)n1] | 2-(1H-imidazol-1-yl)-6-methyl-N- ((1r,4r)-4- methylcyclohexyl)pyrimidine-4- carboxamide |
| 120 | 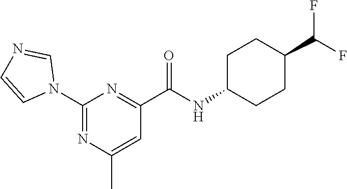 [SMILES: Cc1cc(C(=O)N[C@H]2CC[C@H](C(F)F)CC2)nc(-n2ccnc2)n1] | N-((1r,4r)-4- (difluoromethyl)cyclohexyl)-2-(1H- imidazol-1-yl)-6-methylpyrimidine-4- carboxamide |
| 121 | 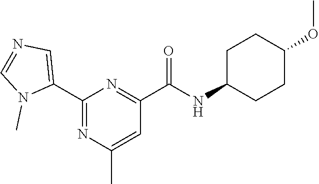 [SMILES: CO[C@H]1CC[C@H](NC(=O)c2cc(C)nc(-c3cncn3C)n2)CC1] | N-((1r,4r)-4-methoxycyclohexyl)-6- methyl-2-(1-methyl-1H-imidazol-5- yl)pyrimidine-4-carboxamide |
| 122 | 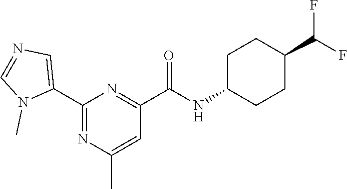 [SMILES: Cc1cc(C(=O)N[C@H]2CC[C@H](C(F)F)CC2)nc(-c2cncn2C)n1] | N-((1r,4r)-4- (difluoromethyl)cyclohexyl)-6- methyl-2-(1-methyl-1H-imidazol-5- yl)pyrimidine-4-carboxamide |
| 123 | 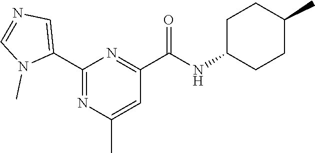 [SMILES: Cc1cc(C(=O)N[C@H]2CC[C@H](C)CC2)nc(-c2cncn2C)n1] | 6-methyl-2-(1-methyl-1H-imidazol-5- yl)-N-((1r,4r)-4- methylcyclohexyl)pyrimidine-4- carboxamide |
| 124 | 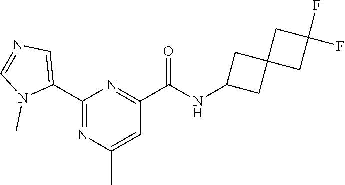 [SMILES: Cc1cc(C(=O)NC2CC3(C2)CC(F)(F)C3)nc(-c2cncn2C)n1] | N-(6,6-difluorospiro[3.3]heptan-2-yl)- 6-methyl-2-(1-methyl-1H-imidazol-5- yl)pyrimidine-4-carboxamide |
| 125 | 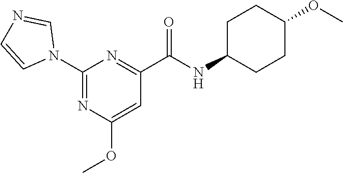 [SMILES: COc1cc(C(=O)N[C@H]2CC[C@H](OC)CC2)nc(-n2ccnc2)n1] | 2-(1H-imidazol-1-yl)-6-methoxy-N- ((1r,4r)-4- methoxycyclohexyl)pyrimidine-4- carboxamide |
| 126 | 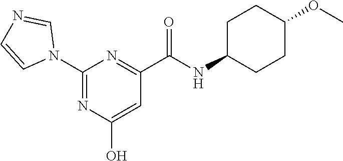 [SMILES: CO[C@H]1CC[C@H](NC(=O)c2cc(O)nc(-n3ccnc3)n2)CC1] | 6-hydroxy-2-(1H-imidazol-1-yl)-N- ((1r,4r)-4- methoxycyclohexyl)pyrimidine-4- carboxamide |
| 127 | 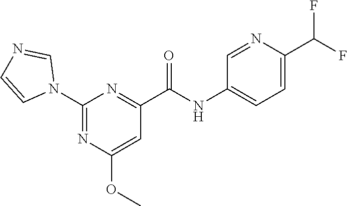 [SMILES: COc1cc(C(=O)Nc2ccc(C(F)F)nc2)nc(-n2ccnc2)n1] | N-(6-(difluoromethyl)pyridin-3-yl)-2- (1H-imidazol-1-yl)-6- methoxypyrimidine-4-carboxamide |
| 128 | 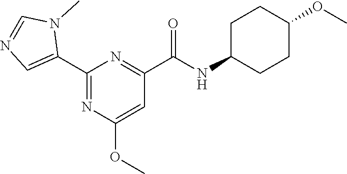 [SMILES: COc1cc(C(=O)N[C@H]2CC[C@H](OC)CC2)nc(-c2cncn2C)n1] | 6-methoxy-N-((1r,4r)-4- methoxycyclohexyl)-2-( 1-methyl-1H- imidazol-5-yl)pyrimidine-4- carboxamide |
| 129 | 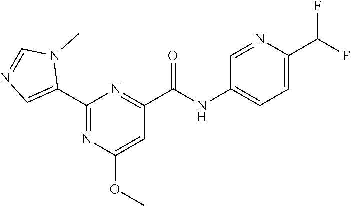 [SMILES: COc1cc(C(=O)Nc2ccc(C(F)F)nc2)nc(-c2cncn2C)n1] | N-(6-(difluoromethyl)pyridin-3-yl)-6- methoxy-2-(1-methyl-1H-imidazol-5- yl)pyrimidine-4-carboxamide |
| 130 | 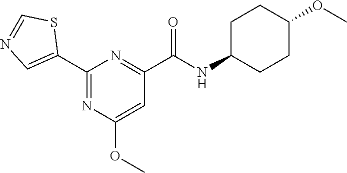 [SMILES: COc1cc(C(=O)N[C@H]2CC[C@H](OC)CC2)nc(-c2cncs2)n1] | 6-methoxy-N-((1r,4r)-4- methoxycyclohexyl)-2-(thiazol-5- yl)pyrimidine-4-carboxamide |
| 131 | 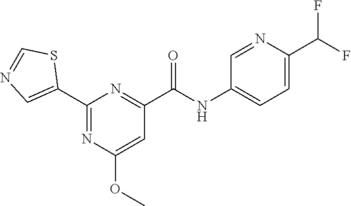 [SMILES: COc1cc(C(=O)Nc2ccc(C(F)F)nc2)nc(-c2cncs2)n1] | N-(6-(difluoromethyl)pyridin-3-yl)-6- methoxy-2-(thiazol-5-yl)pyrimidine- 4-carboxamide |
| 132 | 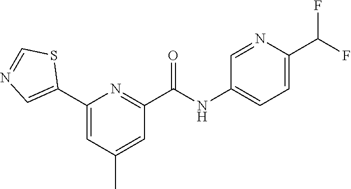 [SMILES: Cc1cc(C(=O)Nc2ccc(C(F)F)nc2)nc(-c2cncs2)c1] | N-(6-(difluoromethyl)pyridin-3-yl)-4- methyl-6-(thiazol-5-yl)picolinamide |
| 133 | 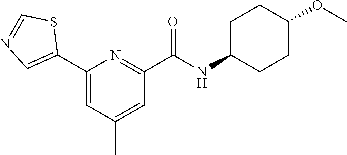 [SMILES: CO[C@H]1CC[C@H](NC(=O)c2cc(C)cc(-c3cncs3)n2)CC1] | N-((1r,4r)-4-methoxycyclohexyl)-4- methyl-6-(thiazol-5-yl)picolinamide |
| 134 | 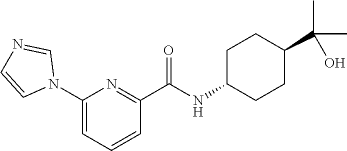 [SMILES: CC(C)(O)[C@H]1CC[C@H](NC(=O)c2cccc(-n3ccnc3)n2)CC1] | N-((1r,4r)-4-(2-hydroxypropan-2- yl)cyclohexyl)-6-(1H-imidazol-1- yl)picolinamide |
| 135 | 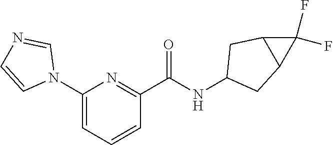 [SMILES: O=C(NC1CC2C(C1)C2(F)F)c1cccc(-n2ccnc2)n1] | N-(6,6-difluorobicyclo[3.1.0]hexan-3- yl)-6-(1H-imidazol-1-yl)picolinamide |
| 136 | 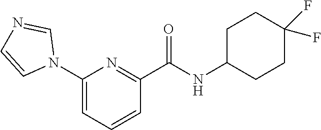 [SMILES: O=C(NC1CCC(F)(F)CC1)c1cccc(-n2ccnc2)n1] | N-(4,4-difluorocyclohexyl)-6-(1H- imidazol-1-yl)picolinamide |
| 137 | 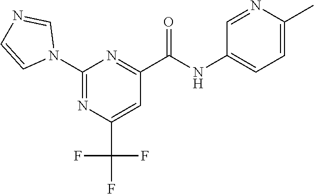 [SMILES: Cc1ccc(NC(=O)c2cc(C(F)(F)F)nc(-n3ccnc3)n2)cn1] | 2-(1H-imidazol-1-yl)-N-(6- methylpyridin-3-yl)-6- (trifluoromethyl)pyrimidine-4- carboxamide |
| 138 | 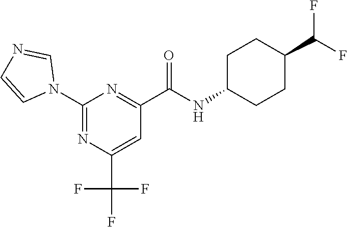 [SMILES: O=C(N[C@H]1CC[C@H](C(F)F)CC1)c1cc(C(F)(F)F)nc(-n2ccnc2)n1] | N-((1r,4r)-4- (difluoromethyl)cyclohexyl)-2-(1H- imidazol-1-yl)-6- (trifluoromethyl)pyrimidine-4- carboxamide |
| 139 | 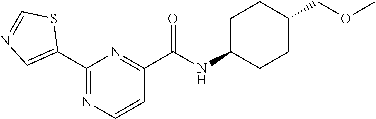 [SMILES: COC[C@H]1CC[C@H](NC(=O)c2ccnc(-c3cncs3)n2)CC1] | N-((1r,4r)-4- (methoxymethyl)cyclohexyl)-2- (thiazol-5-yl)pyrimidine-4- carboxamide |
| 140 | 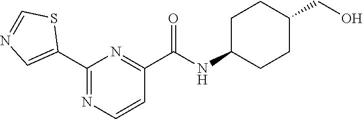 [SMILES: O=C(N[C@H]1CC[C@H](CO)CC1)c1ccnc(-c2cncs2)n1] | N-((1r,4r)-4- (hydroxymethyl)cyclohexyl)-2- (thiazol-5-yl)pyrimidine-4- carboxamide |
| 141 | 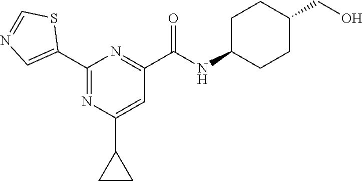 [SMILES: O=C(N[C@H]1CC[C@H](CO)CC1)c1cc(C2CC2)nc(-c2cncs2)n1] | 6-cyclopropyl-N-((1r,4r)-4- (hydroxymethyl)cyclohexyl)-2- (thiazol-5-yl)pyrimidine-4- carboxamide |
| 142 | 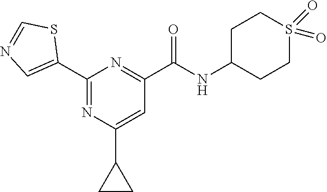 [SMILES: O=C(NC1CCS(=O)(=O)CC1)c1cc(C2CC2)nc(-c2cncs2)n1] | 6-cyclopropyl-N-(1,1- dioxidotetrahydro-2H-thiopyran-4- yl)-2-(thiazol-5-yl)pyrimidine-4- carboxamide |
| 143 | 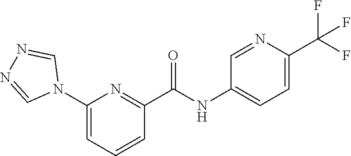 [SMILES: O=C(Nc1ccc(C(F)(F)F)nc1)c1cccc(-n2cnnc2)n1] | 6-(4H-1,2,4-triazol-4-yl)-N-(6- (trifluoromethyl)pyridin-3- yl)picolinamide |
| 144 | 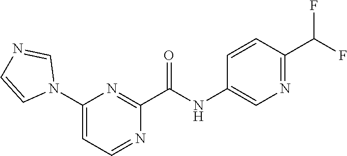 [SMILES: O=C(Nc1ccc(C(F)F)nc1)c1nccc(-n2ccnc2)n1] | N-(6-(difluoromethyl)pyridin-3-yl)-4- (1H-imidazol-1-yl)pyrimidine-2- carboxamide |
| 145 | 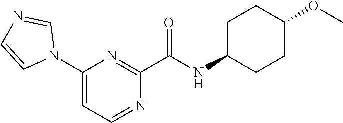 [SMILES: CO[C@H]1CC[C@H](NC(=O)c2nccc(-n3ccnc3)n2)CC1] | 4-(1H-imidazol-1-yl)-N-((1r,4r)-4- methoxycyclohexyl)pyrimidine-2- carboxamide |
| 146 | 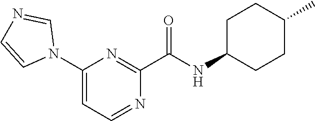 [SMILES: C[C@H]1CC[C@H](NC(=O)c2nccc(-n3ccnc3)n2)CC1] | 4-(1H-imidazol-1-yl)-N-((1r,4r)-4- methylcyclohexyl)pyrimidine-2- carboxamide |
| 147 | 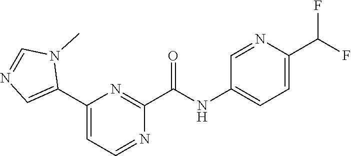 [SMILES: Cn1cncc1-c1ccnc(C(=O)Nc2ccc(C(F)F)nc2)n1] | N-(6-(difluoromethyl)pyridin-3-yl)-4- (1-methyl-1H-imidazol-5- yl)pyrimidine-2-carboxamide |
| 148 | 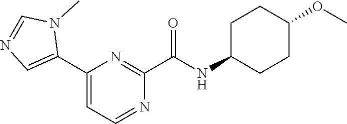 [SMILES: CO[C@H]1CC[C@H](NC(=O)c2nccc(-c3cncn3C)n2)CC1] | N-((1r,4r)-4-methoxycyclohexyl)-4- (1-methyl-1H-imidazol-5- yl)pyrimidine-2-carboxamide |
| 149 | 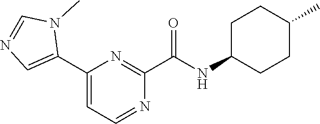 [SMILES: Cn1cncc1-c1ccnc(C(=O)N[C@H]2CC[C@H](C)CC2)n1] | 4-(1-methyl-1H-imidazol-5-yl)-N- ((1r,4r)-4- methylcyclohexyl)pyrimidine-2- carboxamide |
| 150 | 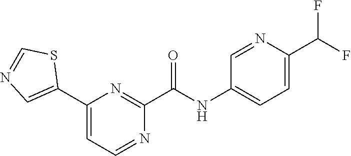 [SMILES: O=C(Nc1ccc(C(F)F)nc1)c1nccc(-c2cncs2)n1] | N-(6-(difluoromethyl)pyridin-3-yl)-4- (thiazol-5-yl)pyrimidine-2- carboxamide |
| 151 | 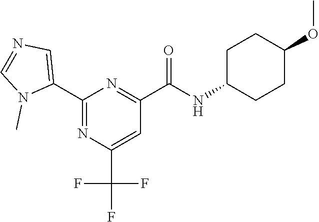 [SMILES: CO[C@H]1CC[C@H](NC(=O)c2cc(C(F)(F)F)nc(-c3cncn3C)n2)CC1] | N-((1r,4r)-4-methoxycyclohexyl)-2- (1-methyl-1H-imidazol-5-yl)-6- (trifluoromethyl)pyrimidine-4- carboxamide |
| 152 | 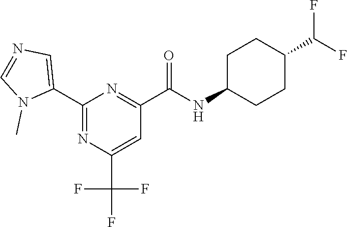 [SMILES: Cn1cncc1-c1nc(C(=O)N[C@H]2CC[C@H](C(F)F)CC2)cc(C(F)(F)F)n1] | N-((1r,4r)-4- (difluoromethyl)cyclohexyl)-2-(1- methyl-1H-imidazol-5-yl)-6- (trifluoromethyl)pyrimidine-4- carboxamide |
| 153 | 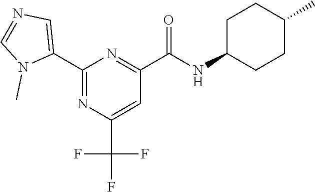 [SMILES: Cn1cncc1-c1nc(C(=O)N[C@H]2CC[C@H](C)CC2)cc(C(F)(F)F)n1] | 2-(1-methyl-1H-imidazol-5-yl)-N- ((1r,4r)-4-methylcyclohexyl)-6- (trifluoromethyl)pyrimidine-4- carboxamide |
| 154 | 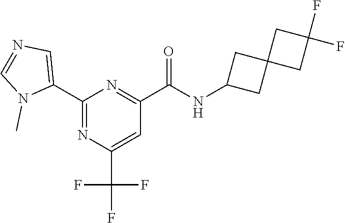 [SMILES: Cn1cncc1-c1nc(C(=O)NC2CC3(C2)CC(F)(F)C3)cc(C(F)(F)F)n1] | N-(6,6-difluorospiro[3.3]heptan-2-yl)- 2-(1-methyl-1H-imidazol-5-yl)-6- (trifluoromethyl)pyrimidine-4- carboxamide |
| 155 | 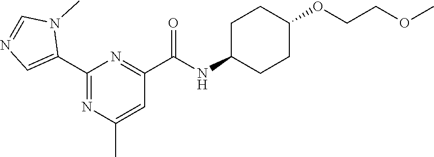 [SMILES: COCCO[C@H]1CC[C@H](NC(=O)c2cc(C)nc(-c3cncn3C)n2)CC1] | N-((1r,4r)-4-(2- methoxyethoxy)cyclohexyl)-6- methyl-2-(1-methyl-1H-imidazol-5- yl)pyrimidine-4-carboxamide |
| 156 | 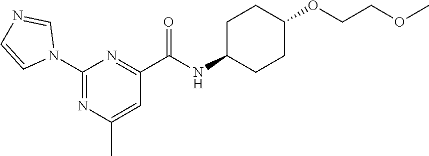 [SMILES: COCCO[C@H]1CC[C@H](NC(=O)c2cc(C)nc(-n3ccnc3)n2)CC1] | 2-(1H-imidazol-1-yl)-N-((1r,4r)-4-(2- methoxyethoxy)cyclohexyl)-6- methylpyrimidine-4-carboxamide |
| 157 | 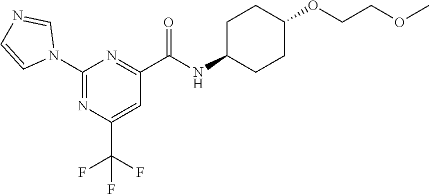 [SMILES: COCCO[C@H]1CC[C@H](NC(=O)c2cc(C(F)(F)F)nc(-n3ccnc3)n2)CC1] | 2-(1H-imidazol-1-yl)-N-((1r,4r)-4-(2- methoxyethoxy)cyclohexyl)-6- (trifluoromethyl)pyrimidine-4- carboxamide |
| 158 | 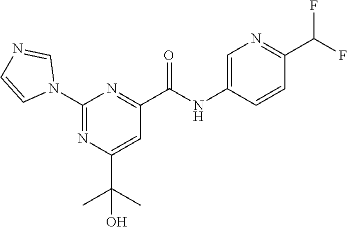 [SMILES: CC(C)(O)c1cc(C(=O)Nc2ccc(C(F)F)nc2)nc(-n2ccnc2)n1] | N-(6-(difluoromethyl)pyridin-3-yl)-6- (2-hydroxypropan-2-yl)-2-(1H- imidazol-1-yl)pyrimidine-4- carboxamide |
| 159 | 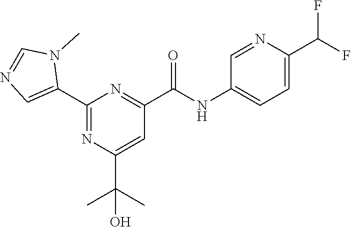 [SMILES: Cn1cncc1-c1nc(C(=O)Nc2ccc(C(F)F)nc2)cc(C(C)(C)O)n1] | N-(6-(difluoromethyl)pyridin-3-yl)-6- (2-hydroxypropan-2-yl)-2-(1-methyl- 1H-imidazol-5-yl)pyrimidine-4- carboxamide |
| 160 | 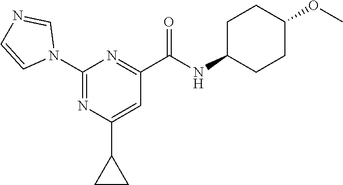 [SMILES: CO[C@H]1CC[C@H](NC(=O)c2cc(C3CC3)nc(-n3ccnc3)n2)CC1] | 6-cyclopropyl-2-(1H-imidazol-1-yl)- N-((1r,4r)-4- methoxycyclohexyl)pyrimidine-4- carboxamide |
| 161 | 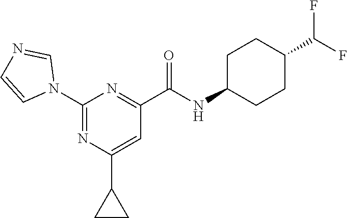 [SMILES: O=C(N[C@H]1CC[C@H](C(F)F)CC1)c1cc(C2CC2)nc(-n2ccnc2)n1] | 6-cyclopropyl-N-((1r,4r)-4- (difluoromethyl)cyclohexyl)-2-(1H- imidazol-1-yl)pyrimidine-4- carboxamide |
| 162 | 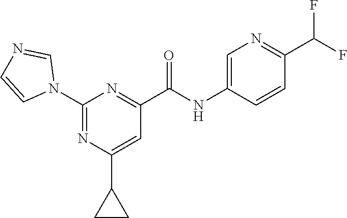 [SMILES: O=C(Nc1ccc(C(F)F)nc1)c1cc(C2CC2)nc(-n2ccnc2)n1] | 6-cyclopropyl-N-(6- (difluoromethyl)pyridin-3-yl)-2-(1H- imidazol-1-yl)pyrimidine-4- carboxamide |
| 163 | 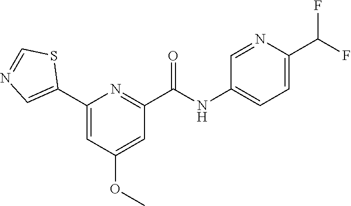 [SMILES: COc1cc(C(=O)Nc2ccc(C(F)F)nc2)nc(-c2cncs2)c1] | N-(6-(difluoromethyl)pyridin-3-yl)-4- methoxy-6-(thiazol-5-yl)picolinamide |
| 164 | 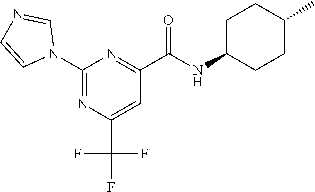 [SMILES: C[C@H]1CC[C@H](NC(=O)c2cc(C(F)(F)F)nc(-n3ccnc3)n2)CC1] | 2-(1H-imidazol-1-yl)-N-((1r,4r)-4- methylcyclohexyl)-6- (trifluoromethyl)pyrimidine-4- carboxamide |
| 165 | 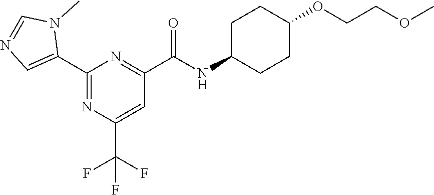 [SMILES: COCCO[C@H]1CC[C@H](NC(=O)c2cc(C(F)(F)F)nc(-c3cncn3C)n2)CC1] | N-((1r,4r)-4-(2- methoxyethoxy)cyclohexyl)-2-(1- methyl-1H-imidazol-5-yl)-6- (trifluoromethyl)pyrimidine-4- carboxamide |
| 166 | 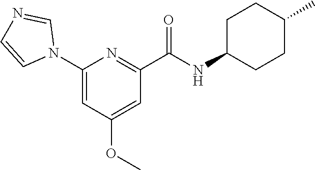 [SMILES: COc1cc(C(=O)N[C@H]2CC[C@H](C)CC2)nc(-n2ccnc2)c1] | 6-(1H-imidazol-1-yl)-4-methoxy-N- ((1r,4r)-4- methylcyclohexyl)picolinamide |
| 167 | 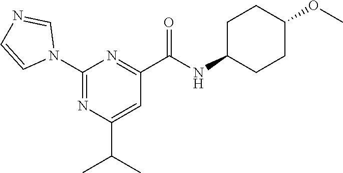 [SMILES: CO[C@H]1CC[C@H](NC(=O)c2cc(C(C)C)nc(-n3ccnc3)n2)CC1] | 2-(1H-imidazol-1-yl)-6-isopropyl-N- ((1r,4r)-4- methoxycyclohexyl)pyrimidine-4- carboxamide |
| 168 | 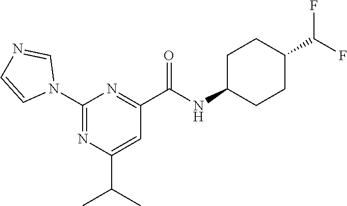 [SMILES: CC(C)c1cc(C(=O)N[C@H]2CC[C@H](C(F)F)CC2)nc(-n2ccnc2)n1] | N-((1r,4r)-4- (difluoromethyl)cyclohexyl)-2-(1H- imidazol-1-yl)-6-isopropylpyrimidine- 4-carboxamide |
| 169 | 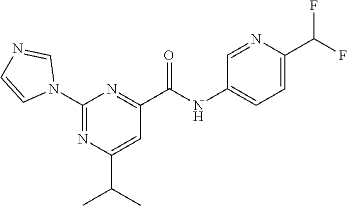 [SMILES: CC(C)c1cc(C(=O)Nc2ccc(C(F)F)nc2)nc(-n2ccnc2)n1] | N-(6-(difluoromethyl)pyridin-3-yl)-2- (1H-imidazol-1-yl)-6- isopropylpyrimidine-4-carboxamide |
| 170 | 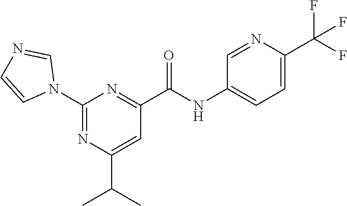 [SMILES: CC(C)c1cc(C(=O)Nc2ccc(C(F)(F)F)nc2)nc(-n2ccnc2)n1] | 2-(1H-imidazol-1-yl)-6-isopropyl-N- (6-(trifluoromethyl)pyridin-3- yl)pyrimidine-4-carboxamide |
| 171 | 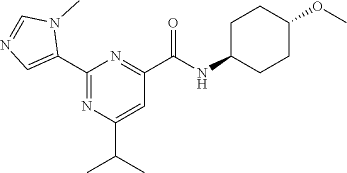 [SMILES: CO[C@H]1CC[C@H](NC(=O)c2cc(C(C)C)nc(-c3cncn3C)n2)CC1] | 6-isopropyl-N-((1r,4r)-4- methoxycyclohexyl)-2-(1-methyl-1H- imidazol-5-yl)pyrimidine-4- carboxamide |
| 172 | 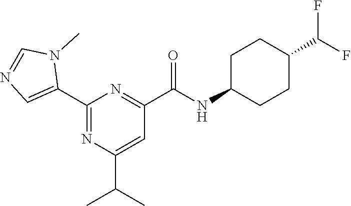 [SMILES: CC(C)c1cc(C(=O)N[C@H]2CC[C@H](C(F)F)CC2)nc(-c2cncn2C)n1] | N-((1r,4r)-4- (difluoromethyl)cyclohexyl)-6- isopropyl-2-(1-methyl-1H-imidazol-5- yl)pyrimidine-4-carboxamide |
| 173 | 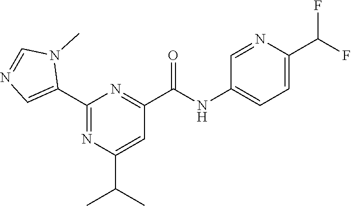 [SMILES: CC(C)c1cc(C(=O)Nc2ccc(C(F)F)nc2)nc(-c2cncn2C)n1] | N-(6-(difluoromethyl)pyridin-3-yl)-6- isopropyl-2-(1-methyl-1H-imidazol-5- yl)pyrimidine-4-carboxamide |
| 174 | 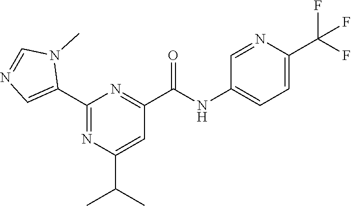 [SMILES: CC(C)c1cc(C(=O)Nc2ccc(C(F)(F)F)nc2)nc(-c2cncn2C)n1] | 6-isopropyl-2-(1-methyl-1H-imidazol- 5-yl)-N-(6-(trifluoromethyl)pyridin-3- yl)pyrimidine-4-carboxamide |
| 175 | 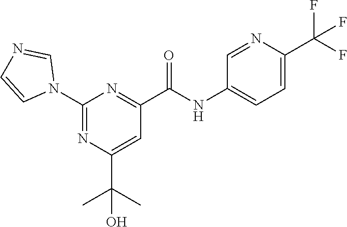 [SMILES: CC(C)(O)c1cc(C(=O)Nc2ccc(C(F)(F)F)nc2)nc(-n2ccnc2)n1] | 6-(2-hydroxypropan-2-yl)-2-(1H- imidazol-1-yl)-N-(6- (trifluoromethyl)pyridin-3- yl)pyrimidine-4-carboxamide |
| 176 | 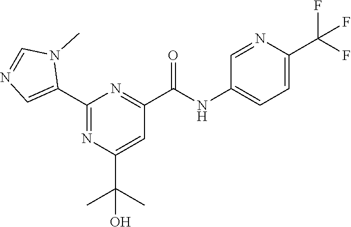 [SMILES: Cn1cncc1-c1nc(C(=O)Nc2ccc(C(F)(F)F)nc2)cc(C(C)(C)O)n1] | 6-(2-hydroxypropan-2-yl)-2-(1- methyl-1H-imidazol-5-yl)-N-(6- (trifluoromethyl)pyridin-3- yl)pyrimidine-4-carboxamide |
| 177 | 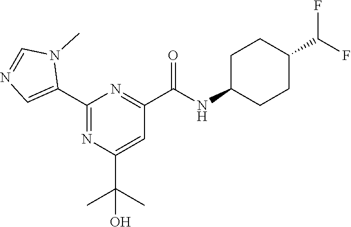 [SMILES: Cn1cncc1-c1nc(C(=O)N[C@H]2CC[C@H](C(F)F)CC2)cc(C(C)(C)O)n1] | N-((1r,4r)-4- (difluoromethyl)cyclohexyl)-6-(2- hydroxypropan-2-yl)-2-(1-methyl-1H- imidazol-5-yl)pyrimidine-4- carboxamide |
| 178 | 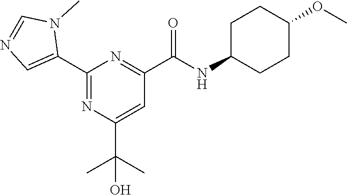 [SMILES: CO[C@H]1CC[C@H](NC(=O)c2cc(C(C)(C)O)nc(-c3cncn3C)n2)CC1] | 6-(2-hydroxypropan-2-yl)-N-((1r,4r)- 4-methoxycyclohexyl)-2-(1-methyl- 1H-imidazol-5-yl)pyrimidine-4- carboxamide |
| 179 | 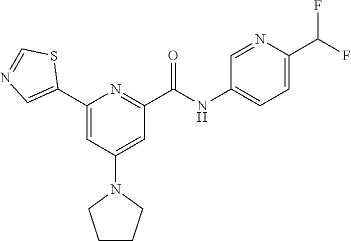 [SMILES: O=C(Nc1ccc(C(F)F)nc1)c1cc(N2CCCC2)cc(-c2cncs2)n1] | N-(6-(difluoromethyl)pyridin-3-yl)-4- (pyrrolidin-1-yl)-6-(thiazol-5- yl)picolinamide |
| 180 | 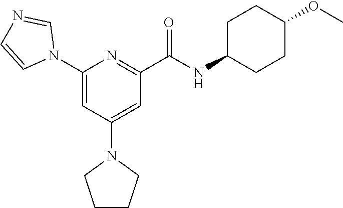 [SMILES: CO[C@H]1CC[C@H](NC(=O)c2cc(N3CCCC3)cc(-n3ccnc3)n2)CC1] | 6-(1H-imidazol-1-yl)-N-((1r,4r)-4- methoxycyclohexyl)-4-(pyrrolidin-1- yl)picolinamide |
| 181 | 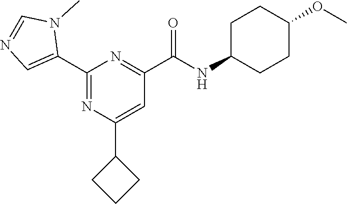 [SMILES: CO[C@H]1CC[C@H](NC(=O)c2cc(C3CCC3)nc(-c3cncn3C)n2)CC1] | 6-cyclobutyl-N-((1r,4r)-4- methoxycyclohexyl)-2-(1-methyl-1H- imidazol-5-yl)pyrimidine-4- carboxamide |
| 182 | 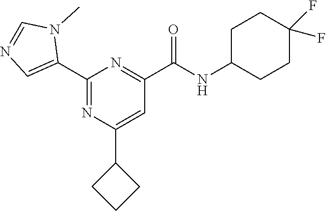 [SMILES: Cn1cncc1-c1nc(C(=O)NC2CCC(F)(F)CC2)cc(C2CCC2)n1] | 6-cyclobutyl-N-(4,4- difluorocyclohexyl)-2-(1-methyl-1H- imidazol-5-yl)pyrimidine-4- carboxamide |
| 183 | 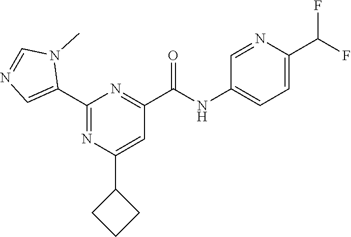 [SMILES: Cn1cncc1-c1nc(C(=O)Nc2ccc(C(F)F)nc2)cc(C2CCC2)n1] | 6-cyclobutyl-N-(6- (difluoromethyl)pyridin-3-yl)-2-(1- methyl-1H-imidazol-5-yl)pyrimidine- 4-carboxamide |
| 184 | 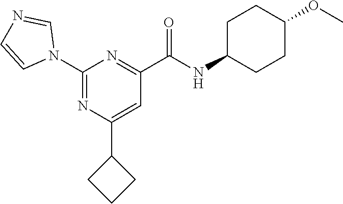 [SMILES: CO[C@H]1CC[C@H](NC(=O)c2cc(C3CCC3)nc(-n3ccnc3)n2)CC1] | 6-cyclobutyl-2-(1H-imidazol-1-yl)-N- ((1r,4r)-4- methoxycyclohexyl)pyrimidine-4- carboxamide |
| 185 | 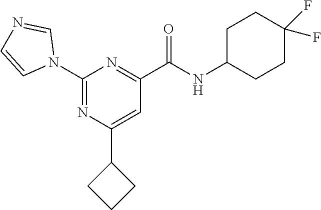 [SMILES: O=C(NC1CCC(F)(F)CC1)c1cc(C2CCC2)nc(-n2ccnc2)n1] | 6-cyclobutyl-N-(4,4- difluorocyclohexyl)-2-(1H-imidazol- 1-yl)pyrimidine-4-carboxamide |
| 186 | 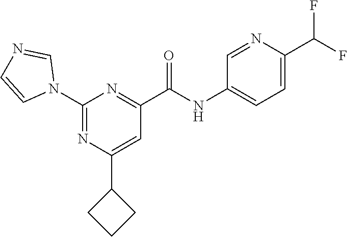 [SMILES: O=C(Nc1ccc(C(F)F)nc1)c1cc(C2CCC2)nc(-n2ccnc2)n1] | 6-cyclobutyl-N-(6- (difluoromethyl)pyridin-3-yl)-2-(1H- imidazol-1-yl)pyrimidine-4- carboxamide |
| 187 | 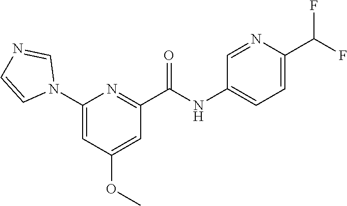 [SMILES: COc1cc(C(=O)Nc2ccc(C(F)F)nc2)nc(-n2ccnc2)c1] | N-(6-(difluoromethyl)pyridin-3-yl)-6- (1H-imidazol-1-yl)-4- methoxypicolinamide |
| 188 | 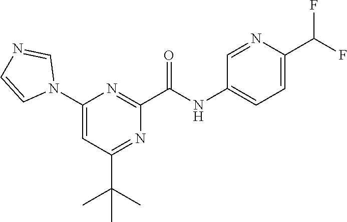 [SMILES: CC(C)(C)c1cc(-n2ccnc2)nc(C(=O)Nc2ccc(C(F)F)nc2)n1] | 4-(tert-butyl)-N-(6- (difluoromethyl)pyridin-3-yl)-6-(1H- imidazol-1-yl)pyrimidine-2- carboxamide |
| 189 | 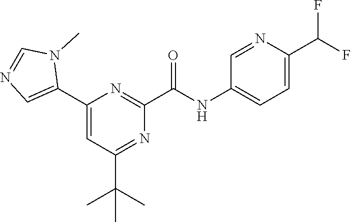 [SMILES: Cn1cncc1-c1cc(C(C)(C)C)nc(C(=O)Nc2ccc(C(F)F)nc2)n1] | 4-(tert-butyl)-N-(6- (difluoromethyl)pyridin-3-yl)-6-(1- methyl-1H-imidazol-5-yl)pyrimidine- 2-carboxamide |
| 190 | 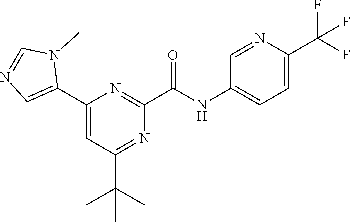 [SMILES: Cn1cncc1-c1cc(C(C)(C)C)nc(C(=O)Nc2ccc(C(F)(F)F)nc2)n1] | 4-(tert-butyl)-6-(1-methyl-1H- imidazol-5-yl)-N-(6- (trifluoromethyl)pyridin-3- yl)pyrimidine-2-carboxamide |
| 191 | 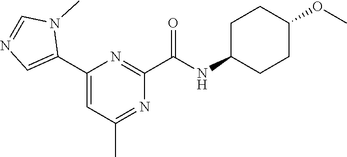 [SMILES: CO[C@H]1CC[C@H](NC(=O)c2nc(C)cc(-c3cncn3C)n2)CC1] | N-((1r,4r)-4-methoxycyclohexyl)-4- methyl-6-(1-methyl-1H-imidazol-5- yl)pyrimidine-2-carboxamide |
| 192 | 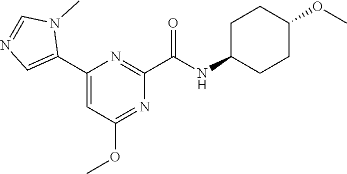 [SMILES: COc1cc(-c2cncn2C)nc(C(=O)N[C@H]2CC[C@H](OC)CC2)n1] | 4-methoxy-N-((1r,4r)-4- methoxycyclohexyl)-6-(1-methyl-1H- imidazol-5-yl)pyrimidine-2- carboxamide |
| 193 | 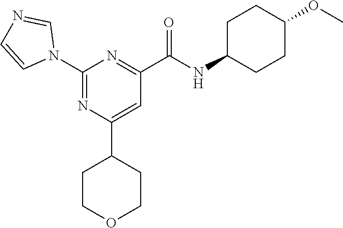 [SMILES: CO[C@H]1CC[C@H](NC(=O)c2cc(C3CCOCC3)nc(-n3ccnc3)n2)CC1] | 2-(1H-imidazol-1-yl)-N-((1r,4r)-4- methoxycyclohexyl)-6-(tetrahydro- 2H-pyran-4-yl)pyrimidine-4- carboxamide |
| 194 | 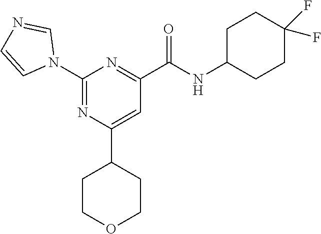 [SMILES: O=C(NC1CCC(F)(F)CC1)c1cc(C2CCOCC2)nc(-n2ccnc2)n1] | N-(4,4-difluorocyclohexyl)-2-(1H- imidazol-1-yl)-6-(tetrahydro-2H- pyran-4-yl)pyrimidine-4-carboxamide |
| 195 | 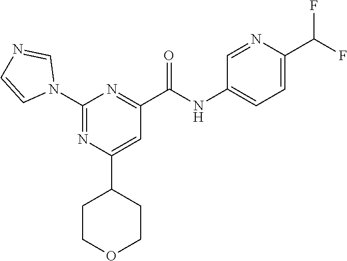 [SMILES: O=C(Nc1ccc(C(F)F)nc1)c1cc(C2CCOCC2)nc(-n2ccnc2)n1] | N-(6-(difluoromethyl)pyridin-3-yl)-2- (1H-imidazol-1-yl)-6-(tetrahydro-2H- pyran-4-yl)pyrimidine-4-carboxamide |
| 196 | 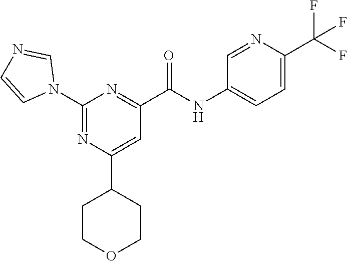 [SMILES: O=C(Nc1ccc(C(F)(F)F)nc1)c1cc(C2CCOCC2)nc(-n2ccnc2)n1] | 2-(1H-imidazol-1-yl)-6-(tetrahydro- 2H-pyran-4-yl)-N-(6- (trifluoromethyl)pyridin-3- yl)pyrimidine-4-carboxamide |
| 197 | 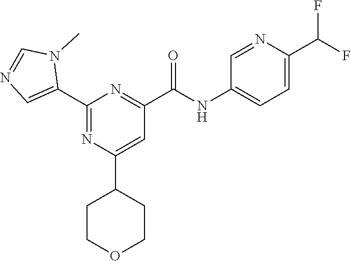 [SMILES: Cn1cncc1-c1nc(C(=O)Nc2ccc(C(F)F)nc2)cc(C2CCOCC2)n1] | N-(6-(difluoromethyl)pyridin-3-yl)-2- (1-methyl-1H-imidazol-5-yl)-6- (tetrahydro-2H-pyran-4- yl)pyrimidine-4-carboxamide |
| 198 | 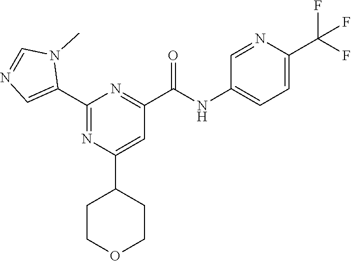 [SMILES: Cn1cncc1-c1nc(C(=O)Nc2ccc(C(F)(F)F)nc2)cc(C2CCOCC2)n1] | 2-(1-methyl-1H-imidazol-5-yl)-6- (tetrahydro-2H-pyran-4-yl)-N-(6- (trifluoromethyl)pyridin-3- yl)pyrimidine-4-carboxamide |
| 199 | 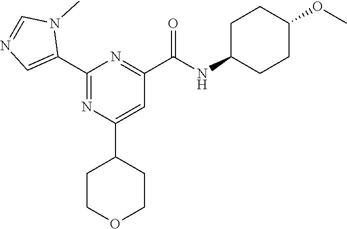 [SMILES: CO[C@H]1CC[C@H](NC(=O)c2cc(C3CCOCC3)nc(-c3cncn3C)n2)CC1] | N-((1r,4r)-4-methoxycyclohexyl)-2- (1-methyl-1H-imidazol-5-yl)-6- (tetrahydro-2H-pyran-4- yl)pyrimidine-4-carboxamide |
| 200 | 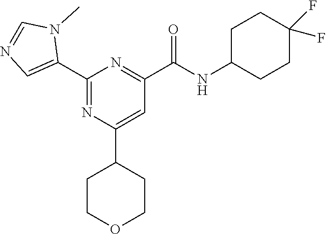 [SMILES: Cn1cncc1-c1nc(C(=O)NC2CCC(F)(F)CC2)cc(C2CCOCC2)n1] | N-(4,4-difluorocyclohexyl)-2-(1- methyl-1H-imidazol-5-yl)-6- (tetrahydro-2H-pyran-4- yl)pyrimidine-4-carboxamide |
| 201 | 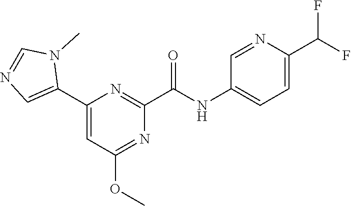 [SMILES: COc1cc(-c2cncn2C)nc(C(=O)Nc2ccc(C(F)F)nc2)n1] | N-(6-(difluoromethyl)pyridin-3-yl)-4- methoxy-6-(1-methyl-1H-imidazol-5- yl)pyrimidine-2-carboxamide |
| 202 | 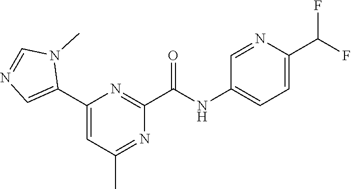 [SMILES: Cc1cc(-c2cncn2C)nc(C(=O)Nc2ccc(C(F)F)nc2)n1] | N-(6-(difluoromethyl)pyridin-3-yl)-4- methyl-6-(1-methyl-1H-imidazol-5- yl)pyrimidine-2-carboxamide |
| 203 | 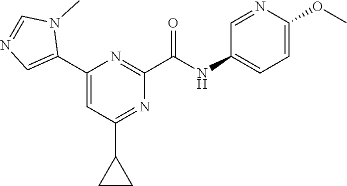 | 4-cyclopropyl-N-((1r,4r)-4- methoxycyclohexyl)-6-(1-methyl-1H- imidazol-5-yl)pyrimidine-2- carboxamide |
| 204 | 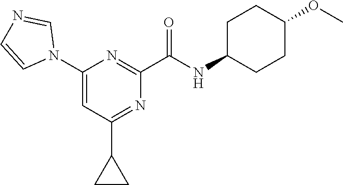 [SMILES: CO[C@H]1CC[C@H](NC(=O)c2nc(C3CC3)cc(-n3ccnc3)n2)CC1] | 4-cyclopropyl-6-(1H-imidazol-1-yl)- N-((1r,4r)-4- methoxycyclohexyl)pyrimidine-2- carboxamide |
| 205 | 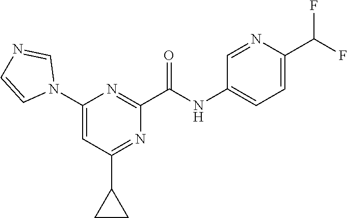 [SMILES: O=C(Nc1ccc(C(F)F)nc1)c1nc(C2CC2)cc(-n2ccnc2)n1] | 4-cyclopropyl-N-(6- (difluoromethyl)pyridin-3-yl)-6-(1H- imidazol-1-yl)pyrimidine-2- carboxamide |
| 206 | 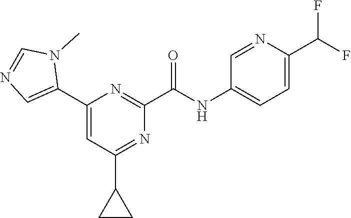 [SMILES: Cn1cncc1-c1cc(C2CC2)nc(C(=O)Nc2ccc(C(F)F)nc2)n1] | 4-cyclopropyl-N-(6- (difluoromethyl)pyridin-3-yl)-6-(1- methyl-1H-imidazol-5-yl)pyrimidine- 2-carboxamide |
| 207 | 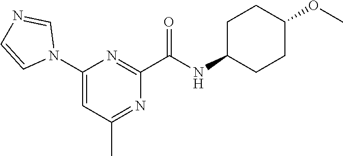 [SMILES: CO[C@H]1CC[C@H](NC(=O)c2nc(C)cc(-n3ccnc3)n2)CC1] | 4-(1H-imidazol-1-yl)-N-((1r,4r)-4- methoxycyclohexyl)-6- methylpyrimidine-2-carboxamide |
| 208 | 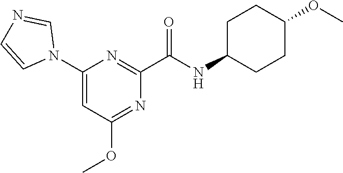 [SMILES: COc1cc(-n2ccnc2)nc(C(=O)N[C@H]2CC[C@H](OC)CC2)n1] | 4-(1H-imidazol-1-yl)-6-methoxy-N- ((1r,4r)-4- methoxycyclohexyl)pyrimidine-2- carboxamide |
| 209 | 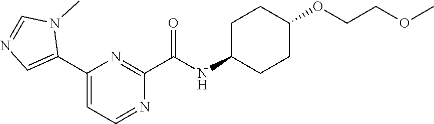 [SMILES: COCCO[C@H]1CC[C@H](NC(=O)c2nccc(-c3cncn3C)n2)CC1] | N-((1r,4r)-4-(2- methoxyethoxy)cyclohexyl)-4-(1- methyl-1H-imidazol-5-yl)pyrimidine- 2-carboxamide |
| 210 | 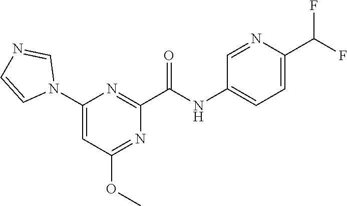 [SMILES: COc1cc(-n2ccnc2)nc(C(=O)Nc2ccc(C(F)F)nc2)n1] | N-(6-(difluoromethyl)pyridin-3-yl)-4- (1H-imidazol-1-yl)-6- methoxypyrimidine-2-carboxamide |
| 211 | 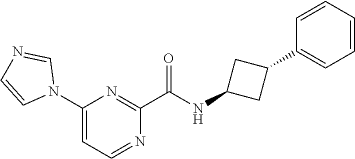 [SMILES: O=C(N[C@H]1C[C@H](c2ccccc2)C1)c1nccc(-n2ccnc2)n1] | 4-(1H-imidazol-1-yl)-N-((1r,3r)-3- phenylcyclobutyl)pyrimidine-2- carboxamide |
| 212 | 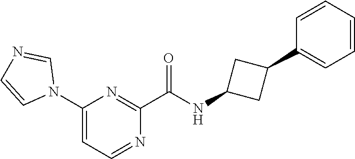 [SMILES: O=C(N[C@H]1C[C@@H](c2ccccc2)C1)c1nccc(-n2ccnc2)n1] | 4-(1H-imidazol-1-yl)-N-((1s,3s)-3- phenylcyclobutyl)pyrimidine-2- carboxamide |
| 213 | 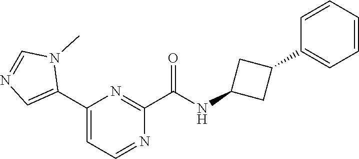 [SMILES: Cn1cncc1-c1ccnc(C(=O)N[C@H]2C[C@H](c3ccccc3)C2)n1] | 4-(1-methyl-1H-imidazol-5-yl)-N- ((1r,3r)-3- phenylcyclobutyl)pyrimidine-2- carboxamide |
| 214 | 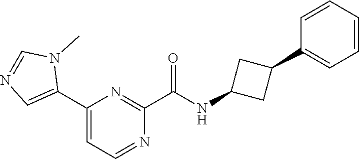 [SMILES: Cn1cncc1-c1ccnc(C(=O)N[C@H]2C[C@@H](c3ccccc3)C2)n1] | 4-(1-methyl-1H-imidazol-5-yl)-N- ((1s,3s)-3- phenylcyclobutyl)pyrimidine-2- carboxamide |
| 215 | 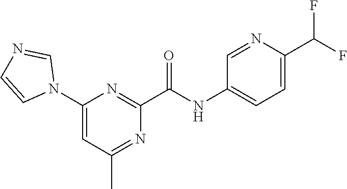 [SMILES: Cc1cc(-n2ccnc2)nc(C(=O)Nc2ccc(C(F)F)nc2)n1] | N-(6-(difluoromethyl)pyridin-3-yl)-4- (1H-imidazol-1-yl)-6- methylpyrimidine-2-carboxamide |
| 216 | 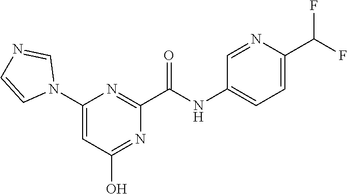 [SMILES: O=C(Nc1ccc(C(F)F)nc1)c1nc(O)cc(-n2ccnc2)n1] | N-(6-(difluoromethyl)pyridin-3-yl)-4- hydroxy-6-(1H-imidazol-1- yl)pyrimidine-2-carboxamide |
| 217 | 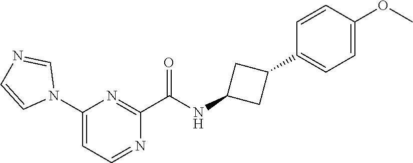 [SMILES: COc1ccc([C@H]2C[C@H](NC(=O)c3nccc(-n4ccnc4)n3)C2)cc1] | 4-(1H-imidazol-1-yl)-N-((1r,3r)-3-(4- methoxyphenyl)cyclobutyl)pyrimidine- 2-carboxamide |
| 218 | 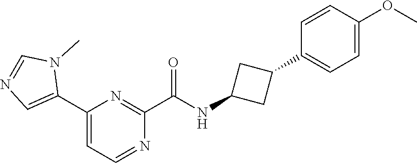 [SMILES: COc1ccc([C@H]2C[C@H](NC(=O)c3nccc(-c4cncn4C)n3)C2)cc1] | N-((1r,3r)-3-(4- methoxyphenyl)cyclobutyl)-4-(1- methyl-1H-imidazol-5-yl)pyrimidine- 2-carboxamide |
| 219 | 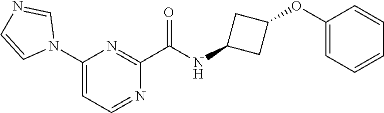 [SMILES: O=C(N[C@H]1C[C@H](Oc2ccccc2)C1)c1nccc(-n2ccnc2)n1] | 4-(1H-imidazol-1-yl)-N-((1r,3r)-3- phenoxycyclobutyl)pyrimidine-2- carboxamide |
| 220 | 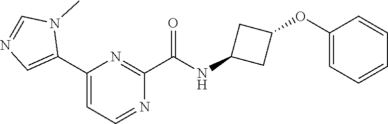 [SMILES: Cn1cncc1-c1ccnc(C(=O)N[C@H]2C[C@H](Oc3ccccc3)C2)n1] | 4-(1-methyl-1H-imidazol-5-yl)-N- ((1r,3r)-3- phenoxycyclobutyl)pyrimidine-2- carboxamide |
| 221 | 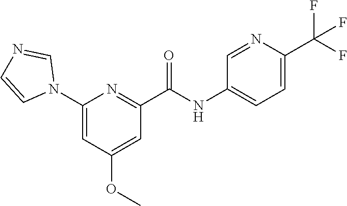 [SMILES: COc1cc(C(=O)Nc2ccc(C(F)(F)F)nc2)nc(-n2ccnc2)c1] | 6-(1H-imidazol-1-yl)-4-methoxy-N- (6-(trifluoromethyl)pyridin-3- yl)picolinamide |
| 222 | 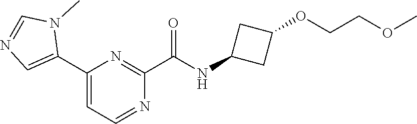 [SMILES: COCCO[C@H]1C[C@H](NC(=O)c2nccc(-c3cncn3C)n2)C1] | N-((1r,3r)-3-(2- methoxyethoxy)cyclobutyl)-4-(1- methyl-1H-imidazol-5-yl)pyrimidine- 2-carboxamide |
| 223 | 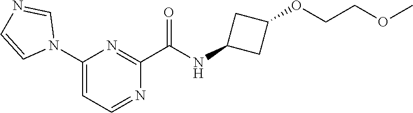 [SMILES: COCCO[C@H]1C[C@H](NC(=O)c2nccc(-n3ccnc3)n2)C1] | 4-(1H-imidazol-1-yl)-N-((1r,3r)-3-(2- methoxyethoxy)cyclobutyl)pyrimidine- 2-carboxamide |
| 224 | 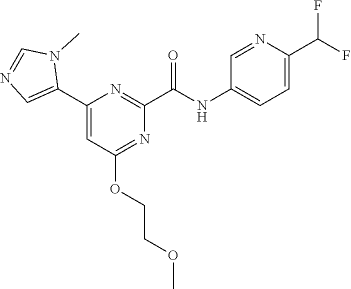 [SMILES: COCCOc1cc(-c2cncn2C)nc(C(=O)Nc2ccc(C(F)F)nc2)n1] | N-(6-(difluoromethyl)pyridin-3-yl)-4- (2-methoxyethoxy)-6-(1-methyl-1H- imidazol-5-yl)pyrimidine-2- carboxamide |
| 225 | 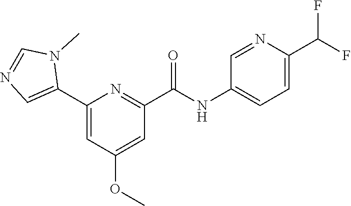 [SMILES: COc1cc(C(=O)Nc2ccc(C(F)F)nc2)nc(-c2cncn2C)c1] | N-(6-(difluoromethyl)pyridin-3-yl)-4- methoxy-6-(1-methyl-1H-imidazol-5- yl)picolinamide |
| 226 | 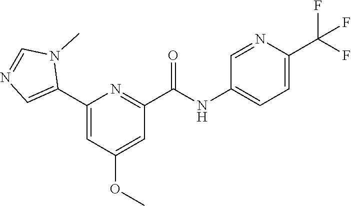 [SMILES: COc1cc(C(=O)Nc2ccc(C(F)(F)F)nc2)nc(-c2cncn2C)c1] | 4-methoxy-6-(1-methyl-1H-imidazol- 5-yl)-N-(6-(trifluoromethyl)pyridin-3- yl)picolinamide |
| 227 | 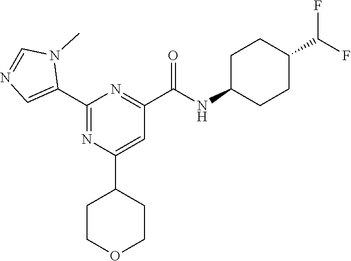 [SMILES: Cn1cncc1-c1nc(C(=O)N[C@H]2CC[C@H](C(F)F)CC2)cc(C2CCOCC2)n1] | N-((1r,4r)-4- (difluoromethyl)cyclohexyl)-2-(1- methyl-1H-imidazol-5-yl)-6- (tetrahydro-2H-pyran-4- yl)pyrimidine-4-carboxamide |
| 228 | 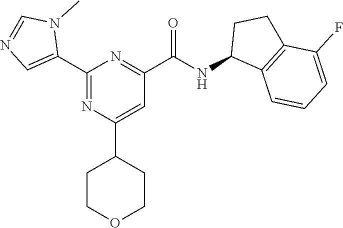 [SMILES: Cn1cncc1-c1nc(C(=O)N[C@H]2CCc3c(F)cccc32)cc(C2CCOCC2)n1] | (S)-N-(4-fluoro-2,3-dihydro-1H- inden-1-yl)-2-(1-methyl-1H-imidazol- 5-yl)-6-(tetrahydro-2H-pyran-4- yl)pyrimidine-4-carboxamide |
| 229 | 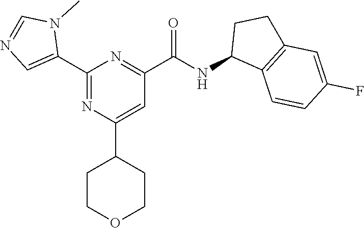 [SMILES: Cn1cncc1-c1nc(C(=O)N[C@H]2CCc3cc(F)ccc32)cc(C2CCOCC2)n1] | (S)-N-(5-fluoro-2,3-dihydro-1H- inden-1-yl)-2-(1-methyl-1H-imidazol- 5-yl)-6-(tetrahydro-2H-pyran-4- yl)pyrimidine-4-carboxamide |
| 230 | 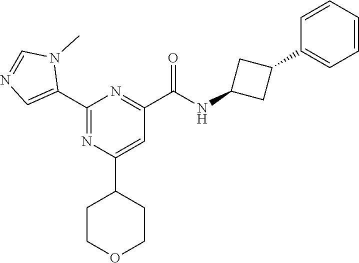 [SMILES: Cn1cncc1-c1nc(C(=O)N[C@H]2C[C@H](c3ccccc3)C2)cc(C2CCOCC2)n1] | 2-(1-methyl-1H-imidazol-5-yl)-N- ((1r,3r)-3-phenylcyclobutyl)-6- (tetrahydro-2H-pyran-4- yl)pyrimidine-4-carboxamide |
| 231 | 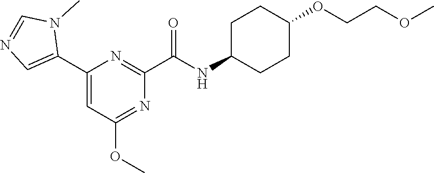 [SMILES: COCCO[C@H]1CC[C@H](NC(=O)c2nc(OC)cc(-c3cncn3C)n2)CC1] | 4-methoxy-N-((1r,4r)-4-(2- methoxyethoxy)cyclohexyl)-6-(1- methyl-1H-imidazol-5-yl)pyrimidine- 2-carboxamide |
| 232 | 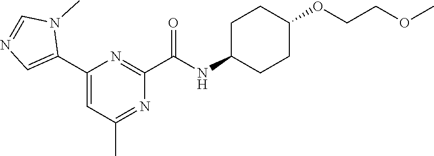 [SMILES: COCCO[C@H]1CC[C@H](NC(=O)c2nc(C)cc(-c3cncn3C)n2)CC1] | N-((1r,4r)-4-(2- methoxyethoxy)cyclohexyl)-4- methyl-6-(1-methyl-1H-imidazol-5- yl)pyrimidine-2-carboxamide |
| 233 | 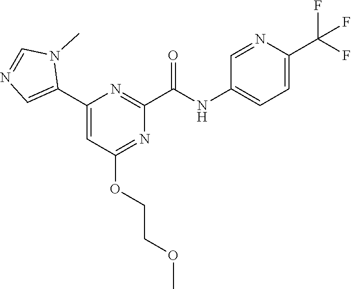 [SMILES: COCCOc1cc(-c2cncn2C)nc(C(=O)Nc2ccc(C(F)(F)F)nc2)n1] | 4-(2-methoxyethoxy)-6-(1-methyl- 1H-imidazol-5-yl)-N-(6- (trifluoromethyl)pyridin-3- yl)pyrimidine-2-carboxamide |
| 234 | 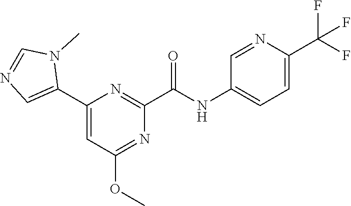 [SMILES: COc1cc(-c2cncn2C)nc(C(=O)Nc2ccc(C(F)(F)F)nc2)n1] | 4-methoxy-6-(1-methyl-1H-imidazol- 5-yl)-N-(6-(trifluoromethyl)pyridin-3- yl)pyrimidine-2-carboxamide |
| 235 | 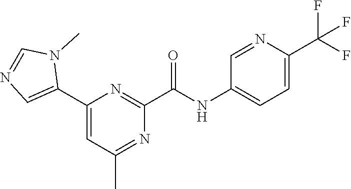 [SMILES: Cc1cc(-c2cncn2C)nc(C(=O)Nc2ccc(C(F)(F)F)nc2)n1] | 4-methyl-6-(1-methyl-1H-imidazol-5- yl)-N-(6-(trifluoromethyl)pyridin-3- yl)pyrimidine-2-carboxamide |
| 236 | 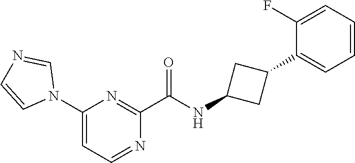 [SMILES: O=C(N[C@H]1C[C@H](c2ccccc2F)C1)c1nccc(-n2ccnc2)n1] | N-((1r,3r)-3-(2- fluorophenyl)cyclobutyl)-4-(1H- imidazol-1-yl)pyrimidine-2- carboxamide |
| 237 | 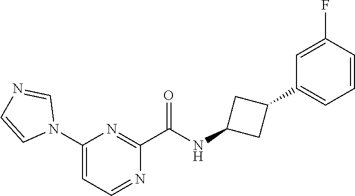 [SMILES: O=C(N[C@H]1C[C@H](c2cccc(F)c2)C1)c1nccc(-n2ccnc2)n1] | N-((1r,3r)-3-(3- fluorophenyl)cyclobutyl)-4-(1H- imidazol-1-yl)pyrimidine-2- carboxamide |
| 238 | 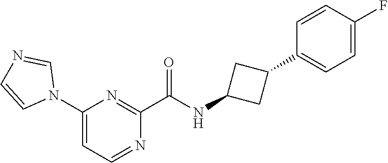 [SMILES: O=C(N[C@H]1C[C@H](c2ccc(F)cc2)C1)c1nccc(-n2ccnc2)n1] | N-((1r,3r)-3-(4- fluorophenyl)cyclobutyl)-4-(1H- imidazol-1-yl)pyrimidine-2- carboxamide |
| 239 | 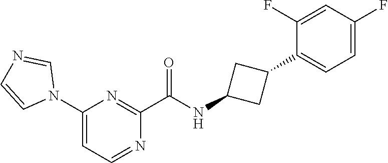 [SMILES: O=C(N[C@H]1C[C@H](c2ccc(F)cc2F)C1)c1nccc(-n2ccnc2)n1] | N-((1r,3r)-3-(2,4- difluorophenyl)cyclobutyl)-4-(1H- imidazol-1-yl)pyrimidine-2- carboxamide |
| 240 | 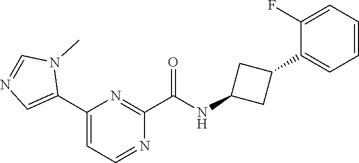 [SMILES: Cn1cncc1-c1ccnc(C(=O)N[C@H]2C[C@H](c3ccccc3F)C2)n1] | N-((1r,3r)-3-(2- fluorophenyl)cyclobutyl)-4-(1- methyl-1H-imidazol-5-yl)pyrimidine- 2-carboxamide |
| 241 | 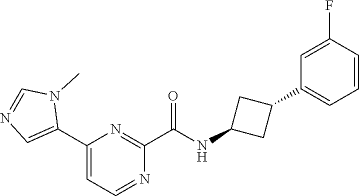 [SMILES: Cn1cncc1-c1ccnc(C(=O)N[C@H]2C[C@H](c3cccc(F)c3)C2)n1] | N-((1r,3r)-3-(3- fluorophenyl)cyclobutyl)-4-(1- methyl-1H-imidazol-5-yl)pyrimidine- 2-carboxamide |
| 242 | 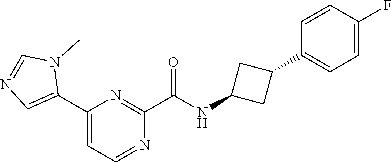 [SMILES: Cn1cncc1-c1ccnc(C(=O)N[C@H]2C[C@H](c3ccc(F)cc3)C2)n1] | N-((1r,3r)-3-(4- fluorophenyl)cyclobutyl)-4-(1- methyl-1H-imidazol-5-yl)pyrimidine- 2-carboxamide |
| 243 | 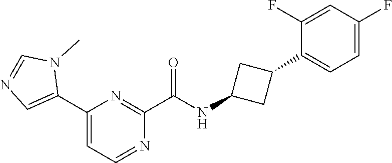 [SMILES: Cn1cncc1-c1ccnc(C(=O)N[C@H]2C[C@H](c3ccc(F)cc3F)C2)n1] | N-((1r,3r)-3-(2,4- difluorophenyl)cyclobutyl)-4-(1- methyl-1H-imidazol-5-yl)pyrimidine- 2-carboxamide |
| 244 | 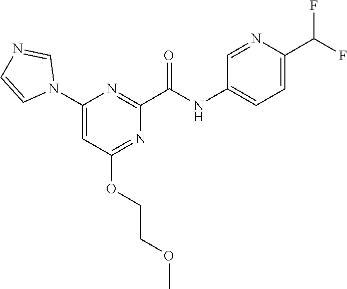 [SMILES: COCCOc1cc(-n2ccnc2)nc(C(=O)Nc2ccc(C(F)F)nc2)n1] | N-(6-(difluoromethyl)pyridin-3-yl)-6- (1H-imidazol-1-yl)-4-(2- methoxyethoxy)picolinamide |
| 245 | 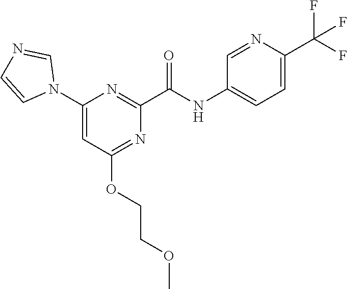 [SMILES: COCCOc1cc(-n2ccnc2)nc(C(=O)Nc2ccc(C(F)(F)F)nc2)n1] | 6-(1H-imidazol-1-yl)-4-(2- methoxyethoxy)-N-(6- (trifluoromethyl)pyridin-3- yl)picolinamide |
| 246 | 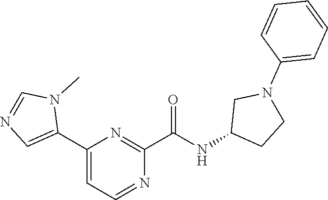 [SMILES: Cn1cncc1-c1ccnc(C(=O)N[C@H]2CCN(c3ccccc3)C2)n1] | (S)-4-(1-methyl-1H-imidazol-5-yl)-N- (1-phenylpyrrolidin-3-yl)pyrimidine- 2-carboxamide |
| 247 | 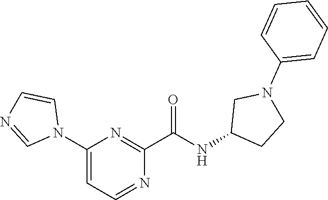 [SMILES: O=C(N[C@H]1CCN(c2ccccc2)C1)c1nccc(-n2ccnc2)n1] | (S)-4-(1H-imidazol-1-yl)-N-(1- phenylpyrrolidin-3-yl)pyrimidine-2- carboxamide |
| 248 | 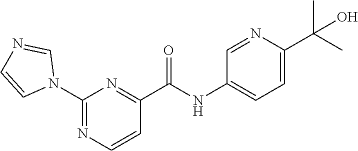 [SMILES: CC(C)(O)c1ccc(NC(=O)c2ccnc(-n3ccnc3)n2)cn1] | N-(6-(2-hydroxypropan-2-yl)pyridin- 3-yl)-2-(1H-imidazol-1- yl)pyrimidine-4-carboxamide |
| 249 | 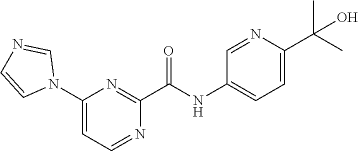 [SMILES: CC(C)(O)c1ccc(NC(=O)c2nccc(-n3ccnc3)n2)cn1] | N-(6-(2-hydroxypropan-2-yl)pyridin- 3-yl)-4-(1H-imidazol-1- yl)pyrimidine-2-carboxamide |
| 250 | 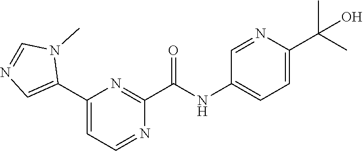 [SMILES: Cn1cncc1-c1ccnc(C(=O)Nc2ccc(C(C)(C)O)nc2)n1] | N-(6-(2-hydroxypropan-2-yl)pyridin- 3-yl)-4-(1-methyl-1H-imidazol-5- yl)pyrimidine-2-carboxamide |
| 251 | 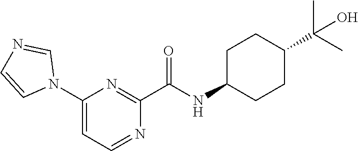 [SMILES: CC(C)(O)[C@H]1CC[C@H](NC(=O)c2nccc(-n3ccnc3)n2)CC1] | N-((1r,4r)-4-(2-hydroxypropan-2- yl)cyclohexyl)-4-(1H-imidazol-1- yl)pyrimidine-2-carboxamide |
| 252 | 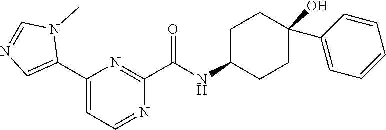 [SMILES: Cn1cncc1-c1ccnc(C(=O)N[C@H]2CC[C@](O)(c3ccccc3)CC2)n1] | N-((1s,4s)-4-hydroxy-4- phenylcyclohexyl)-4-(1-methyl-1H- imidazol-5-yl)pyrimidine-2- carboxamide |
| 253 | 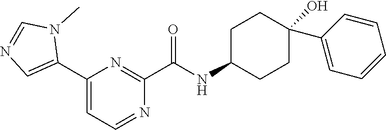 [SMILES: Cn1cncc1-c1ccnc(C(=O)N[C@H]2CC[C@@](O)(c3ccccc3)CC2)n1] | N-((1r,4r)-4-hydroxy-4- phenylcyclohexyl)-4-(1-methyl-1H- imidazol-5-yl)pyrimidine-2- carboxamide |
| 254 | 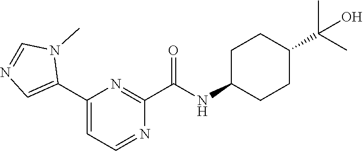 [SMILES: Cn1cncc1-c1ccnc(C(=O)N[C@H]2CC[C@H](C(C)(C)O)CC2)n1] | N-((1r,4r)-4-(2-hydroxypropan-2- yl)cyclohexyl)-4-(1-methyl-1H- imidazol-5-yl)pyrimidine-2- carboxamide |
| 255 | 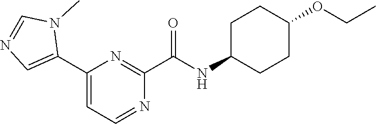 [SMILES: CCO[C@H]1CC[C@H](NC(=O)c2nccc(-c3cncn3C)n2)CC1] | N-((1r,4r)-4-ethoxycyclohexyl)-4-(1- methyl-1H-imidazol-5-yl)pyrimidine- 2-carboxamide |
| 256 | 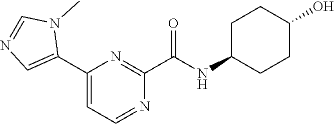 [SMILES: Cn1cncc1-c1ccnc(C(=O)N[C@H]2CC[C@H](O)CC2)n1] | N-((1r,4r)-4-hydroxycyclohexyl)-4- (1-methyl-1H-imidazol-5- yl)pyrimidine-2-carboxamide |
| 257 | 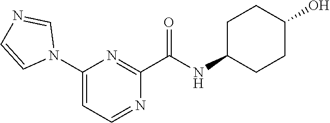 [SMILES: O=C(N[C@H]1CC[C@H](O)CC1)c1nccc(-n2ccnc2)n1] | N-((1r,4r)-4-hydroxycyclohexyl)-4- (1H-imidazol-1-yl)pyrimidine-2- carboxamide |
| 258 | 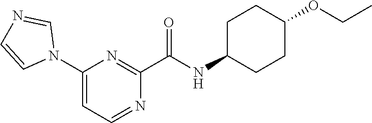 [SMILES: CCO[C@H]1CC[C@H](NC(=O)c2nccc(-n3ccnc3)n2)CC1] | N-((1r,4r)-4-ethoxycyclohexyl)-4- (1H-imidazol-1-yl)pyrimidine-2- carboxamide |
| 259 | 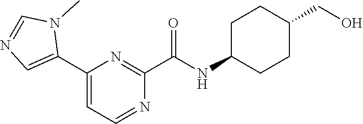 [SMILES: Cn1cncc1-c1ccnc(C(=O)N[C@H]2CC[C@H](CO)CC2)n1] | N-((1r,4r)-4- (hydroxymethyl)cyclohexyl)-4-(1- methyl-1H-imidazol-5-yl)pyrimidine- 2-carboxamide |
| 260 | 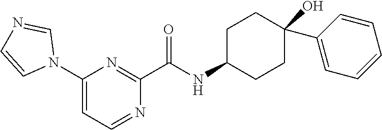 [SMILES: O=C(N[C@H]1CC[C@](O)(c2ccccc2)CC1)c1nccc(-n2ccnc2)n1] | N-((1s,4s)-4-hydroxy-4- phenylcyclohexyl)-4-(1H-imidazol-1- yl)pyrimidine-2-carboxamide |
| 261 | 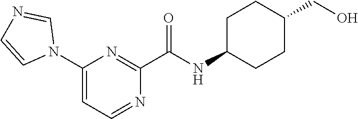 [SMILES: O=C(N[C@H]1CC[C@H](CO)CC1)c1nccc(-n2ccnc2)n1] | N-((1r,4r)-4- (hydroxymethyl)cyclohexyl)-4-(1H- imidazol-1-yl)pyrimidine-2- carboxamide |
| 262 | 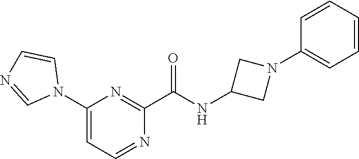 [SMILES: O=C(NC1CN(c2ccccc2)C1)c1nccc(-n2ccnc2)n1] | 4-(1H-imidazol-1-yl)-N-(1- phenylazetidin-3-yl)pyrimidine-2- carboxamide |
| 263 | 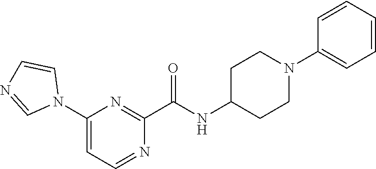 [SMILES: O=C(NC1CCN(c2ccccc2)CC1)c1nccc(-n2ccnc2)n1] | 4-(1H-imidazol-1-yl)-N-(1- phenylpiperidin-4-yl)pyrimidine-2- carboxamide |
| 264 | 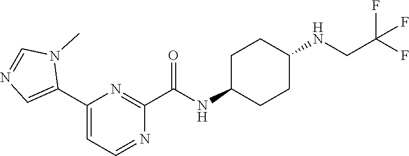 [SMILES: Cn1cncc1-c1ccnc(C(=O)N[C@H]2CC[C@H](NCC(F)(F)F)CC2)n1] | 4-(1-methyl-1H-imidazol-5-yl)-N- ((1r,4r)-4-((2,2,2- trifluoroethyl)amino)cyclohexyl) pyrimidine-2-carboxamide |
| 265 | 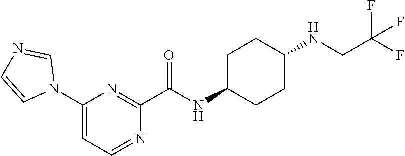 [SMILES: O=C(N[C@H]1CC[C@H](NCC(F)(F)F)CC1)c1nccc(-n2ccnc2)n1] | 4-(1H-imidazol-1-yl)-N-((1r,4r)-4- ((2,2,2- trifluoroethyl)amino)cyclohexyl) pyrimidine-2-carboxamide |
| 266 | 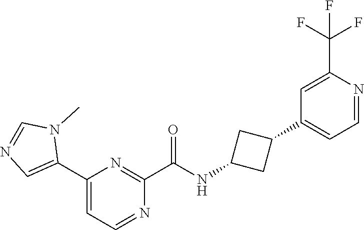 [SMILES: Cn1cncc1-c1ccnc(C(=O)N[C@H]2C[C@@H](c3ccnc(C(F)(F)F)c3)C2)n1] | 4-(1-methyl-1H-imidazol-5-yl)-N- ((1s,3s)-3-(2-(trifluoromethyl)pyridin- 4-yl)cyclobutyl)pyrimidine-2- carboxamide |
| 267 | 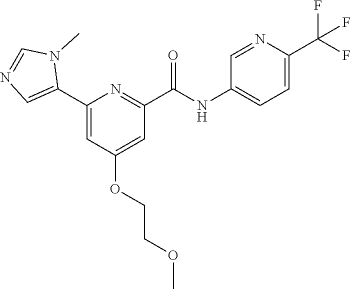 [SMILES: COCCOc1cc(C(=O)Nc2ccc(C(F)(F)F)nc2)nc(-c2cncn2C)c1] | 6-(2-methoxyethoxy)-2-(1-methyl- 1H-imidazol-5-yl)-N-(6- (trifluoromethyl)pyridin-3- yl)pyrimidine-4-carboxamide |
| 268 | 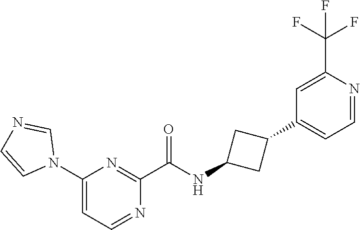 [SMILES: O=C(N[C@H]1C[C@H](c2ccnc(C(F)(F)F)c2)C1)c1nccc(-n2ccnc2)n1] | 4-(1H-imidazol-1-yl)-N-((1r,3r)-3-(2- (trifluoromethyl)pyridin-4- yl)cyclobutyl)pyrimidine-2- carboxamide |
| 269 | 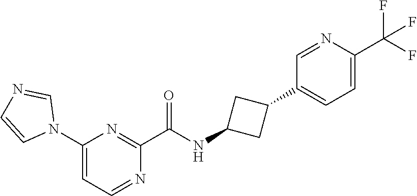 [SMILES: O=C(N[C@H]1C[C@H](c2ccc(C(F)(F)F)nc2)C1)c1nccc(-n2ccnc2)n1] | 4-(1H-imidazol-1-yl)-N-((1r,3r)-3-(6- (trifluoromethyl)pyridin-3- yl)cyclobutyl)pyrimidine-2- carboxamide |
| 270 | 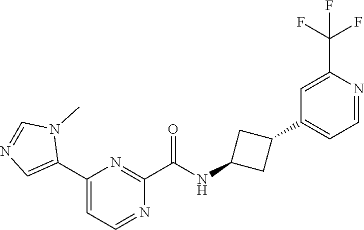 [SMILES: Cn1cncc1-c1ccnc(C(=O)N[C@H]2C[C@H](c3ccnc(C(F)(F)F)c3)C2)n1] | 4-(1-methyl-1H-imidazol-5-yl)-N- ((1r,3r)-3-(2-(trifluoromethyl)pyridin- 4-yl)cyclobutyl)pyrimidine-2- carboxamide |
| 271 | 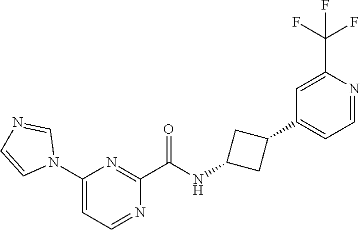 [SMILES: O=C(N[C@H]1C[C@@H](c2ccnc(C(F)(F)F)c2)C1)c1nccc(-n2ccnc2)n1] | 4-(1H-imidazol-1-yl)-N-((1s,3s)-3-(2- (trifluoromethyl)pyridin-4- yl)cyclobutyl)pyrimidine-2- carboxamide |
| 272 | 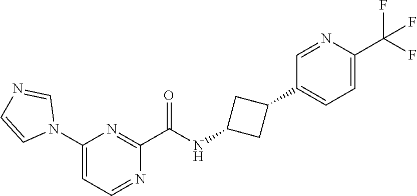 [SMILES: O=C(N[C@H]1C[C@@H](c2ccc(C(F)(F)F)nc2)C1)c1nccc(-n2ccnc2)n1] | 4-(1H-imidazol-1-yl)-N-((1s,3s)-3-(6- (trifluoromethyl)pyridin-3- yl)cyclobutyl)pyrimidine-2- carboxamide |
| 273 | 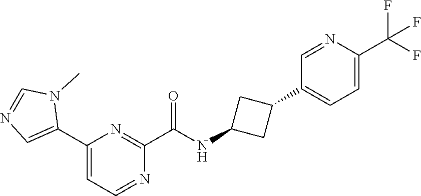 [SMILES: Cn1cncc1-c1ccnc(C(=O)N[C@H]2C[C@H](c3ccc(C(F)(F)F)nc3)C2)n1] | 4-(1-methyl-1H-imidazol-5-yl)-N- ((1r,3r)-3-(6-(trifluoromethyl)pyridin- 3-yl)cyclobutyl)pyrimidine-2- carboxamide |
| 274 | 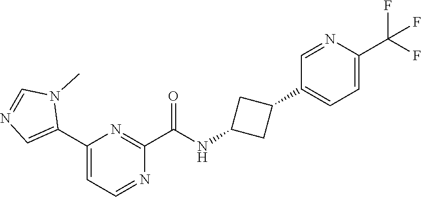 [SMILES: Cn1cncc1-c1ccnc(C(=O)N[C@H]2C[C@@H](c3ccc(C(F)(F)F)nc3)C2)n1] | 4-(1-methyl-1H-imidazol-5-yl)-N- ((1s,3s)-3-(6-(trifluoromethyl)pyridin- 3-yl)cyclobutyl)pyrimidine-2- carboxamide |
| 275 | 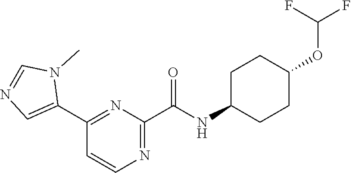 [SMILES: Cn1cncc1-c1ccnc(C(=O)N[C@H]2CC[C@H](OC(F)F)CC2)n1] | N-((1r,4r)-4- (difluoromethoxy)cyclohexyl)-4-(1- methyl-1H-imidazol-5-yl)pyrimidine- 2-carboxamide |
| 276 | 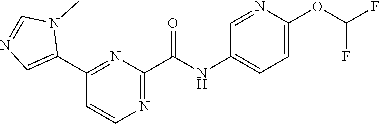 [SMILES: Cn1cncc1-c1ccnc(C(=O)Nc2ccc(OC(F)F)nc2)n1] | N-(6-(difluoromethoxy)pyridin-3-yl)- 4-(1-methyl-1H-imidazol-5- yl)pyrimidine-2-carboxamide |
| 277 | 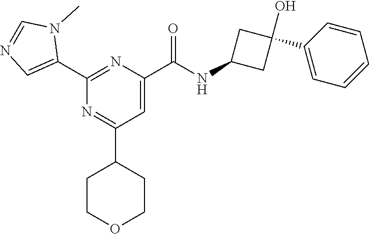 [SMILES: Cn1cncc1-c1nc(C(=O)N[C@H]2C[C@](O)(c3ccccc3)C2)cc(C2CCOCC2)n1] | N-((1s,3s)-3-hydroxy-3- phenylcyclobutyl)-2-(1-methyl-1H- imidazol-5-yl)-6-(tetrahydro-2H- pyran-4-yl)pyrimidine-4-carboxamide |
| 278 | 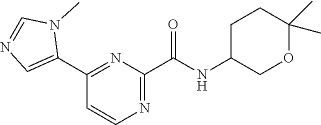 [SMILES: Cn1cncc1-c1ccnc(C(=O)NC2CCC(C)(C)OC2)n1] | N-(6,6-dimethyltetrahydro-2H-pyran- 3-yl)-4-(1-methyl-1H-imidazol-5- yl)pyrimidine-2-carboxamide |
| 279 | 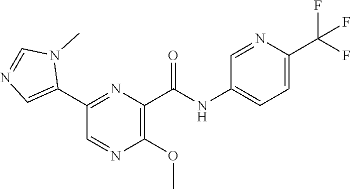 [SMILES: COc1ncc(-c2cncn2C)nc1C(=O)Nc1ccc(C(F)(F)F)nc1] | 3-methoxy-6-(1-methyl-1H-imidazol- 5-yl)-N-(6-(trifluoromethyl)pyridin-3- yl)pyrazine-2-carboxamide |
| 280 | 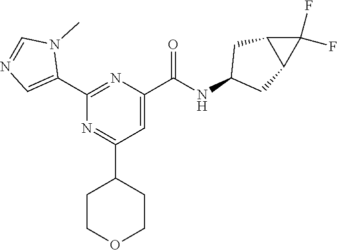 [SMILES: Cn1cncc1-c1nc(C(=O)N[C@@H]2C[C@@H]3[C@H](C2)C3(F)F)cc(C2CCOCC2)n1] | N-((1R,3s,5S)-6,6- difluorobicyclo[3.1.0]hexan-3-yl)-2- (1-methyl-1H-imidazol-5-yl)-6- (tetrahydro-2H-pyran-4- yl)pyrimidine-4-carboxamide |
| 281 | 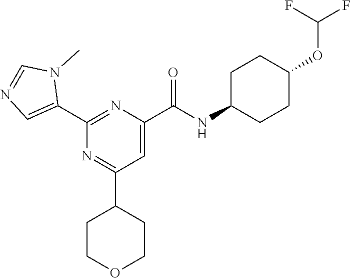 [SMILES: Cn1cncc1-c1nc(C(=O)N[C@H]2CC[C@H](OC(F)F)CC2)cc(C2CCOCC2)n1] | N-((1r,4r)-4- (difluoromethoxy)cyclohexyl)-2-(1- methyl-1H-imidazol-5-yl)-6- (tetrahydro-2H-pyran-4- yl)pyrimidine-4-carboxamide |
| 282 | 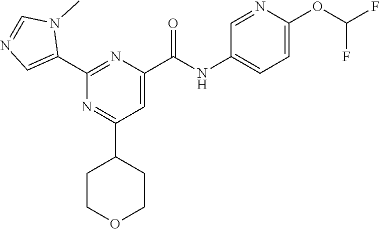 [SMILES: Cn1cncc1-c1nc(C(=O)Nc2ccc(OC(F)F)nc2)cc(C2CCOCC2)n1] | N-(6-(difluoromethoxy)pyridin-3-yl)- 2-(1-methyl-1H-imidazol-5-yl)-6- (tetrahydro-2H-pyran-4- yl)pyrimidine-4-carboxamide |
| 283 | 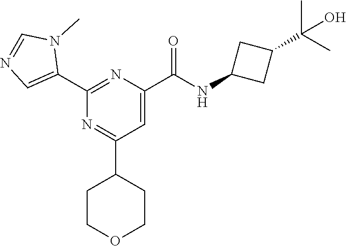 [SMILES: Cn1cncc1-c1nc(C(=O)N[C@H]2C[C@H](C(C)(C)O)C2)cc(C2CCOCC2)n1] | N-((1r,3r)-3-(2-hydroxypropan-2- yl)cyclobutyl)-2-(1-methyl-1H- imidazol-5-yl)-6-(tetrahydro-2H- pyran-4-yl)pyrimidine-4-carboxamide |
| 284 | 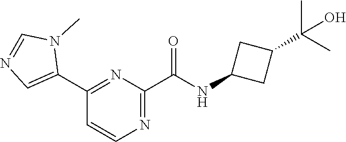 [SMILES: Cn1cncc1-c1ccnc(C(=O)N[C@H]2C[C@H](C(C)(C)O)C2)n1] | N-((1r,3r)-3-(2-hydroxypropan-2- yl)cyclobutyl)-4-(1-methyl-1H- imidazol-5-yl)pyrimidine-2- carboxamide |
| 285 | 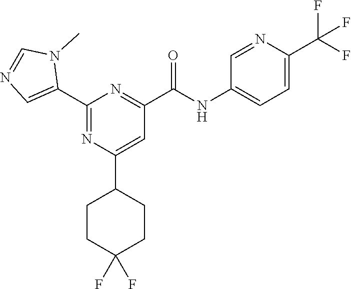 [SMILES: Cn1cncc1-c1nc(C(=O)Nc2ccc(C(F)(F)F)nc2)cc(C2CCC(F)(F)CC2)n1] | 6-(4,4-difluorocyclohexyl)-2-(1- methyl-1H-imidazol-5-yl)-N-(6- (trifluoromethyl)pyridin-3- yl)pyrimidine-4-carboxamide |
| 286 | 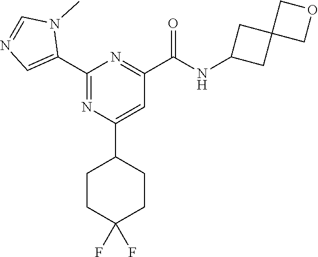 [SMILES: Cn1cncc1-c1nc(C(=O)NC2CC3(COC3)C2)cc(C2CCC(F)(F)CC2)n1] | 6-(4,4-difluorocyclohexyl)-2-(1- methyl-1H-imidazol-5-yl)-N-(2- oxaspiro[3.3]heptan-6-yl)pyrimidine- 4-carboxamide |
| 287 | 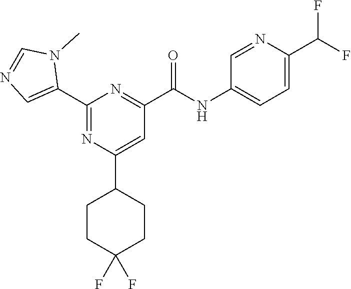 [SMILES: Cn1cncc1-c1nc(C(=O)Nc2ccc(C(F)F)nc2)cc(C2CCC(F)(F)CC2)n1] | 6-(4,4-difluorocyclohexyl)-N-(6- (difluoromethyl)pyridin-3-yl)-2-(1- methyl-1H-imidazol-5-yl)pyrimidine- 4-carboxamide |
| 288 | 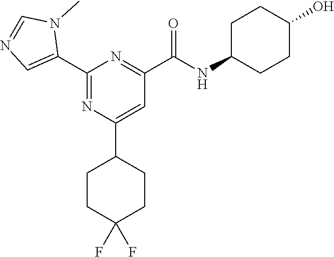 [SMILES: Cn1cncc1-c1nc(C(=O)N[C@H]2CC[C@H](O)CC2)cc(C2CCC(F)(F)CC2)n1] | 6-(4,4-difluorocyclohexyl)-N-((1r,4r)- 4-hydroxycyclohexyl)-2-(1-methyl- 1H-imidazol-5-yl)pyrimidine-4- carboxamide |
| 289 | 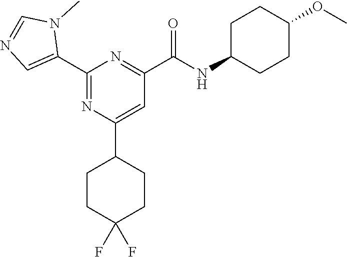 [SMILES: CO[C@H]1CC[C@H](NC(=O)c2cc(C3CCC(F)(F)CC3)nc(-c3cncn3C)n2)CC1] | 6-(4,4-difluorocyclohexyl)-N-((1r,4r)- 4-methoxycyclohexyl)-2-(1-methyl- 1H-imidazol-5-yl)pyrimidine-4- carboxamide |
| 290 | 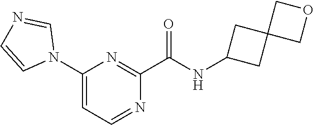 [SMILES: O=C(NC1CC2(COC2)C1)c1nccc(-n2ccnc2)n1] | 4-(1H-imidazol-1-yl)-N-(2- oxaspiro[3.3]heptan-6-yl)pyrimidine- 2-carboxamide |
| 291 | 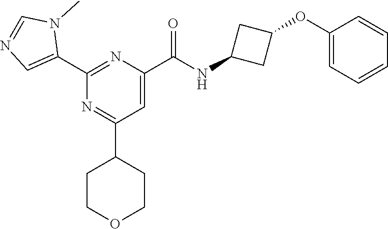 [SMILES: Cn1cncc1-c1nc(C(=O)N[C@H]2C[C@H](Oc3ccccc3)C2)cc(C2CCOCC2)n1] | 2-(1-methyl-1H-imidazol-5-yl)-N- ((1r,3r)-3-phenoxycyclobutyl)-6- (tetrahydro-2H-pyran-4- yl)pyrimidine-4-carboxamide |
| 292 | 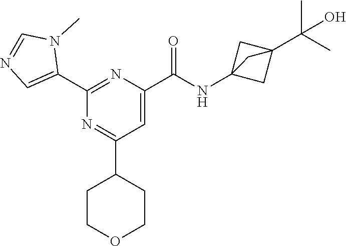 [SMILES: Cn1cncc1-c1nc(C(=O)NC23CC(C(C)(C)O)(C2)C3)cc(C2CCOCC2)n1] | N-(3-(2-hydroxypropan-2- yl)bicyclo[1.1.1]pentan-1-yl)-2-(1- methyl-1H-imidazol-5-yl)-6- (tetrahydro-2H-pyran-4- yl)pyrimidine-4-carboxamide |
| 293 | 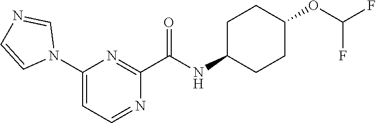 [SMILES: O=C(N[C@H]1CC[C@H](OC(F)F)CC1)c1nccc(-n2ccnc2)n1] | N-((1r,4r)-4- (difluoromethoxy)cyclohexyl)-4-(1H- imidazol-1-yl)pyrimidine-2- carboxamide |
| 294 | 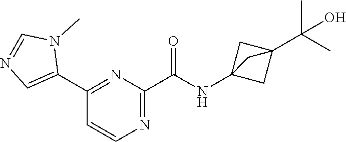 [SMILES: Cn1cncc1-c1ccnc(C(=O)NC23CC(C(C)(C)O)(C2)C3)n1] | N-(3-(2-hydroxypropan-2- yl)bicyclo[1.1.1]pentan-1-yl)-4-(1- methyl-1H-imidazol-5-yl)pyrimidine- 2-carboxamide |
| 295 | 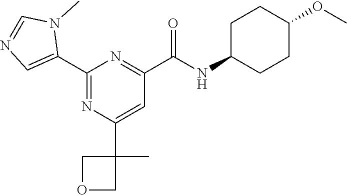 [SMILES: CO[C@H]1CC[C@H](NC(=O)c2cc(C3(C)COC3)nc(-c3cncn3C)n2)CC1] | N-((1r,4r)-4-methoxycyclohexyl)-2- (1-methyl-1H-imidazol-5-yl)-6-(3- methyloxetan-3-yl)pyrimidine-4- carboxamide |
| 296 | 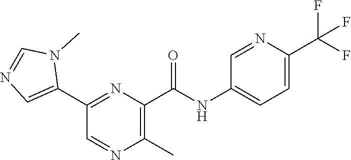 [SMILES: Cc1ncc(-c2cncn2C)nc1C(=O)Nc1ccc(C(F)(F)F)nc1] | 3-methyl-6-(1-methyl-1H-imidazol-5- yl)-N-(6-(trifluoromethyl)pyridin-3- yl)pyrazine-2-carboxamide |
| 297 | 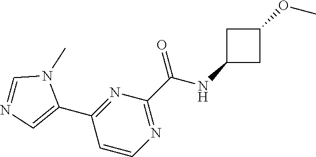 [SMILES: CO[C@H]1C[C@H](NC(=O)c2nccc(-c3cncn3C)n2)C1] | N-((1r,3r)-3-methoxycyclobutyl)-4- (1-methyl-1H-imidazol-5- yl)pyrimidine-2-carboxamide |
| 298 | 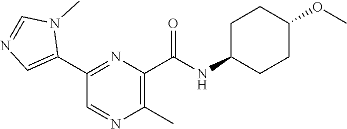 [SMILES: CO[C@H]1CC[C@H](NC(=O)c2nc(-c3cncn3C)cnc2C)CC1] | N-((1r,4r)-4-methoxycyclohexyl)-3- methyl-6-(1-methyl-1H-imidazol-5- yl)pyrazine-2-carboxamide |
| 299 | 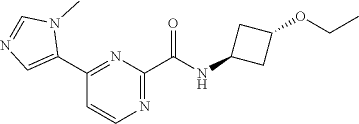 [SMILES: CCO[C@H]1C[C@H](NC(=O)c2nccc(-c3cncn3C)n2)C1] | N-((1r,3r)-3-ethoxycyclobutyl)-4-(1- methyl-1H-imidazol-5-yl)pyrimidine- 2-carboxamide |
| 300 | 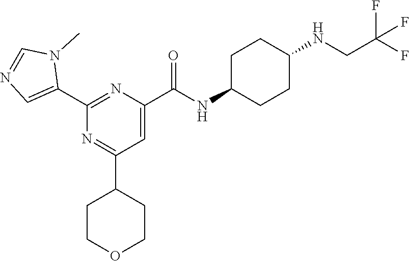 [SMILES: Cn1cncc1-c1nc(C(=O)N[C@H]2CC[C@H](NCC(F)(F)F)CC2)cc(C2CCOCC2)n1] | 2-(1-methyl-1H-imidazol-5-yl)-6- (tetrahydro-2H-pyran-4-yl)-N- ((1r,4r)-4-((2,2,2- trifluoroethyl)amino)cyclohexyl) pyrimidine-4-carboxamide |
| 301 | 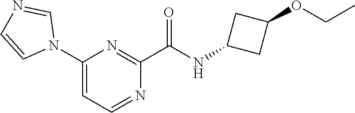 [SMILES: CCO[C@H]1C[C@H](NC(=O)c2nccc(-n3ccnc3)n2)C1] | N-((1r,3r)-3-ethoxycyclobutyl)-4- (1H-imidazol-1-yl)pyrimidine-2- carboxamide |
| 302 | 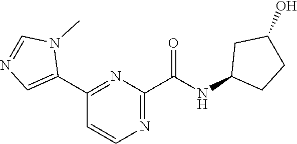 [SMILES: Cn1cncc1-c1ccnc(C(=O)N[C@@H]2CC[C@@H](O)C2)n1] | N-((1R,3R)-3-hydroxycyclopentyl)-4- (1-methyl-1H-imidazol-5- yl)pyrimidine-2-carboxamide |
| 303 | 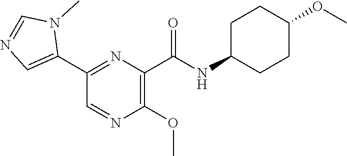 [SMILES: COc1ncc(-c2cncn2C)nc1C(=O)N[C@H]1CC[C@H](OC)CC1] | 3-methoxy-N-((1r,4r)-4- methoxycyclohexyl)-6-(1-methyl-1H- imidazol-5-yl)pyrazine-2- carboxamide |
| 304 | 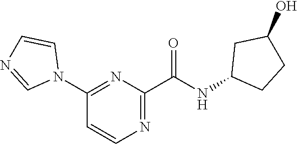 [SMILES: O=C(N[C@H]1CC[C@H](O)C1)c1nccc(-n2ccnc2)n1] | N-((1S,3S)-3-hydroxycyclopentyl)-4- (1H-imidazol-1-yl)pyrimidine-2- carboxamide |
| 305 | 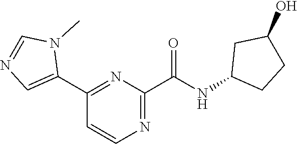 [SMILES: Cn1cncc1-c1ccnc(C(=O)N[C@H]2CC[C@H](O)C2)n1] | N-((1S,3S)-3-hydroxycyclopentyl)-4- (1-methyl-1H-imidazol-5- yl)pyrimidine-2-carboxamide |
| 306 | 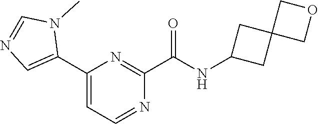 [SMILES: Cn1cncc1-c1ccnc(C(=O)NC2CC3(COC3)C2)n1] | 4-(1-methyl-1H-imidazol-5-yl)-N-(2- oxaspiro[3.3]heptan-6-yl)pyrimidine- 2-carboxamide |
| 307 | 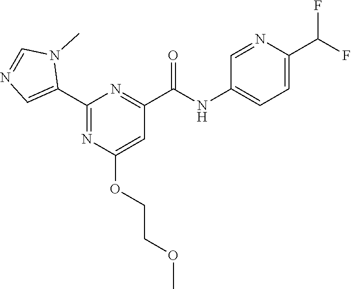 [SMILES: COCCOc1cc(C(=O)Nc2ccc(C(F)F)nc2)nc(-c2cncn2C)n1] | N-(6-(difluoromethyl)pyridin-3-yl)-6- (2-methoxy ethoxy)-2-(1-methyl-1H- imidazol-5-yl)pyrimidine-4- carboxamide |
| 308 | 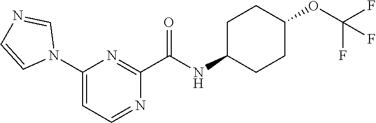 [SMILES: O=C(N[C@H]1CC[C@H](OC(F)(F)F)CC1)c1nccc(-n2ccnc2)n1] | 4-(1H-imidazol-1-yl)-N-((1r,4r)-4- (trifluoromethoxy)cyclohexyl)pyrimidine- 2-carboxamide |
| 309 | 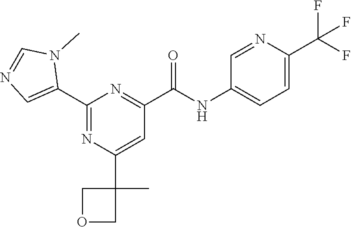 [SMILES: Cn1cncc1-c1nc(C(=O)Nc2ccc(C(F)(F)F)nc2)cc(C2(C)COC2)n1] | 2-(1-methyl-1H-imidazol-5-yl)-6-(3- methyloxetan-3-yl)-N-(6- (trifluoromethyl)pyridin-3- yl)pyrimidine-4-carboxamide |
| 310 | 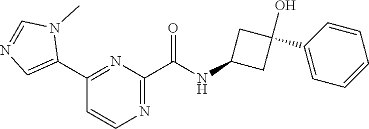 [SMILES: Cn1cncc1-c1ccnc(C(=O)N[C@H]2C[C@](O)(c3ccccc3)C2)n1] | N-((1s,3s)-3-hydroxy-3- phenylcyclobutyl)-4-(1-methyl-1H- imidazol-5-yl)pyrimidine-2- carboxamide |
| 311 | 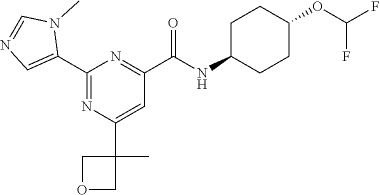 [SMILES: Cn1cncc1-c1nc(C(=O)N[C@H]2CC[C@H](OC(F)F)CC2)cc(C2(C)COC2)n1] | N-((1r,4r)-4- (difluoromethoxy)cyclohexyl)-2-(1- methyl-1H-imidazol-5-yl)-6-(3- methyloxetan-3-yl)pyrimidine-4- carboxamide |
| 312 | 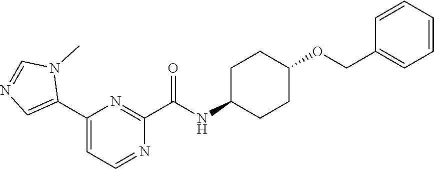 [SMILES: Cn1cncc1-c1ccnc(C(=O)N[C@H]2CC[C@H](OCc3ccccc3)CC2)n1] | N-((1r,4r)-4-(benzyloxy)cyclohexyl)- 4-(1-methyl-1H-imidazol-5- yl)pyrimidine-2-carboxamide |
| 313 | 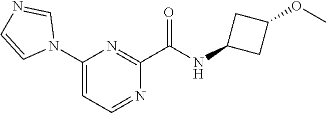 [SMILES: CO[C@H]1C[C@H](NC(=O)c2nccc(-n3ccnc3)n2)C1] | 4-(1H-imidazol-1-yl)-N-((1r,3r)-3- methoxycyclobutyl)pyrimidine-2- carboxamide |
| 314 | 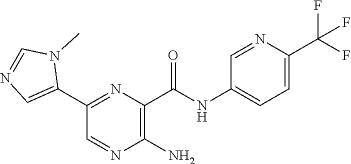 [SMILES: Cn1cncc1-c1cnc(N)c(C(=O)Nc2ccc(C(F)(F)F)nc2)n1] | 3-amino-6-(1-methyl-1H-imidazol-5- yl)-N-(6-(trifluoromethyl)pyridin-3- yl)pyrazine-2-carboxamide |
| 315 | 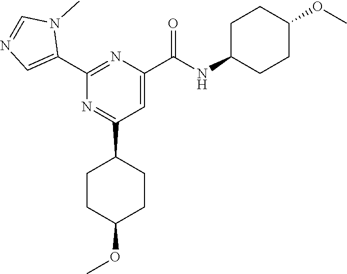 [SMILES: CO[C@H]1CC[C@H](NC(=O)c2cc([C@H]3CC[C@@H](OC)CC3)nc(-c3cncn3C)n2)CC1] | N-((1r,4R)-4-methoxycyclohexyl)-6- ((1s,4S)-4-methoxycyclohexyl)-2-(1- methyl-1H-imidazol-5-yl)pyrimidine- 4-carboxamide |
| 316 | 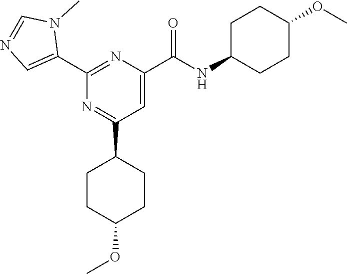 [SMILES: CO[C@H]1CC[C@H](NC(=O)c2cc([C@H]3CC[C@H](OC)CC3)nc(-c3cncn3C)n2)CC1] | N,6-bis((1r,4R)-4- methoxycyclohexyl)-2-(1-methyl-1H- imidazol-5-yl)pyrimidine-4- carboxamide |
| 317 | 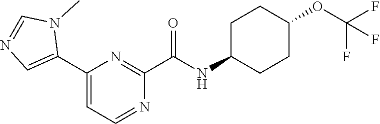 [SMILES: Cn1cncc1-c1ccnc(C(=O)N[C@H]2CC[C@H](OC(F)(F)F)CC2)n1] | 4-(1-methyl-1H-imidazol-5-yl)-N- ((1r,4r)-4- (trifluoromethoxy)cyclohexyl)pyrimidine- 2-carboxamide |
| 318 | 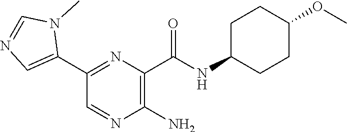 [SMILES: CO[C@H]1CC[C@H](NC(=O)c2nc(-c3cncn3C)cnc2N)CC1] | 3-amino-N-((1r,4r)-4- methoxycyclohexyl)-6-(1-methyl-1H- imidazol-5-yl)pyrazine-2- carboxamide |
| 319 | 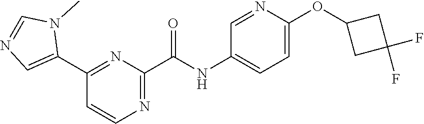 [SMILES: Cn1cncc1-c1ccnc(C(=O)Nc2ccc(OC3CC(F)(F)C3)nc2)n1] | N-(6-(3,3- difluorocyclobutoxy)pyridin-3-yl)-4- (1-methyl-1H-imidazol-5- yl)pyrimidine-2-carboxamide |
| 320 | 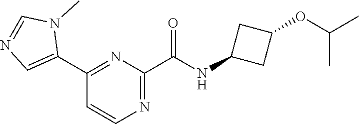 [SMILES: CC(C)O[C@H]1C[C@H](NC(=O)c2nccc(-c3cncn3C)n2)C1] | N-((1r,3r)-3-isopropoxycyclobutyl)-4- (1-methyl-1H-imidazol-5- yl)pyrimidine-2-carboxamide |
| 321 | 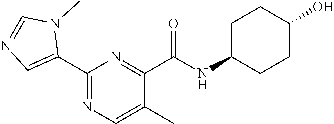 [SMILES: Cc1cnc(-c2cncn2C)nc1C(=O)N[C@H]1CC[C@H](O)CC1] | N-((1r,4r)-4-hydroxycyclohexyl)-5- methyl-2-(1-methyl-1H-imidazol-5- yl)pyrimidine-4-carboxamide |
| 322 | 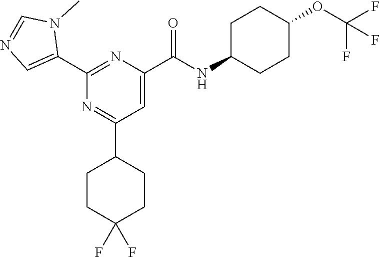 [SMILES: Cn1cncc1-c1nc(C(=O)N[C@H]2CC[C@H](OC(F)(F)F)CC2)cc(C2CCC(F)(F)CC2)n1] | 6-(4,4-difluorocyclohexyl)-2-(1- methyl-1H-imidazol-5-yl)-N-((1r,4r)- 4- (trifluoromethoxy)cyclohexyl)pyrimidine- 4-carboxamide |
| 323 | 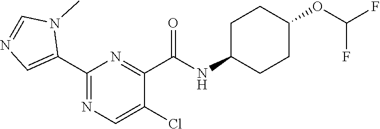 [SMILES: Cn1cncc1-c1ncc(Cl)c(C(=O)N[C@H]2CC[C@H](OC(F)F)CC2)n1] | 5-chloro-N-((1r,4r)-4- (difluoromethoxy)cyclohexyl)-2-(1- methyl-1H-imidazol-5-yl)pyrimidine- 4-carboxamide |
| 324 | 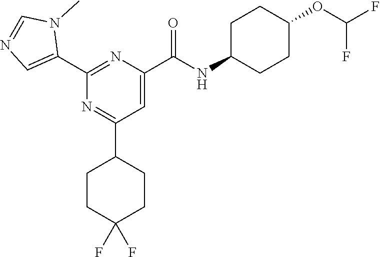 [SMILES: Cn1cncc1-c1nc(C(=O)N[C@H]2CC[C@H](OC(F)F)CC2)cc(C2CCC(F)(F)CC2)n1] | 6-(4,4-difluorocyclohexyl)-N-((1r,4r)- 4-(difluoromethoxy)cyclohexyl)-2-(1- methyl-1H-imidazol-5-yl)pyrimidine- 4-carboxamide |
| 325 | 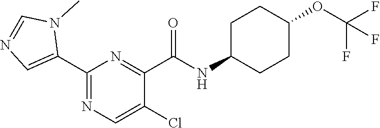 [SMILES: Cn1cncc1-c1ncc(Cl)c(C(=O)N[C@H]2CC[C@H](OC(F)(F)F)CC2)n1] | 5-chloro-2-(1-methyl-1H-imidazol-5- yl)-N-((1r,4r)-4- (trifluoromethoxy)cyclohexyl)pyrimidine- 4-carboxamide |
| 326 | 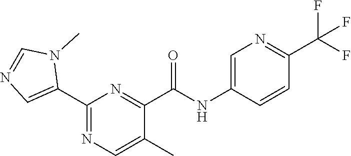 [SMILES: Cc1cnc(-c2cncn2C)nc1C(=O)Nc1ccc(C(F)(F)F)nc1] | 5-methyl-2-(1-methyl-1H-imidazol-5- yl)-N-(6-(trifluoromethyl)pyridin-3- yl)pyrimidine-4-carboxamide |
| 327 | 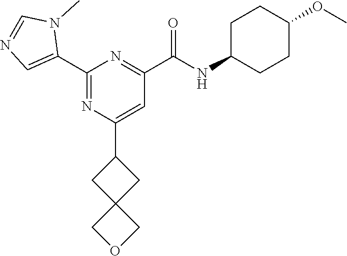 [SMILES: CO[C@H]1CC[C@H](NC(=O)c2cc(C3CC4(COC4)C3)nc(-c3cncn3C)n2)CC1] | N-((1r,4r)-4-methoxycyclohexyl)-2- (1-methyl-1H-imidazol-5-yl)-6-(2- oxaspiro[3.3]heptan-6-yl)pyrimidine- 4-carboxamide |
| 328 | 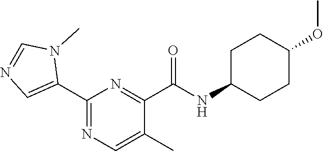 [SMILES: CO[C@H]1CC[C@H](NC(=O)c2nc(-c3cncn3C)ncc2C)CC1] | N-((1r,4r)-4-methoxycyclohexyl)-5- methyl-2-(1-methyl-1H-imidazol-5- yl)pyrimidine-4-carboxamide |
| 329 | 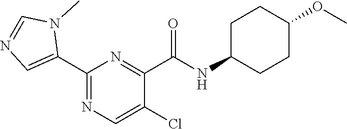 [SMILES: CO[C@H]1CC[C@H](NC(=O)c2nc(-c3cncn3C)ncc2Cl)CC1] | 5-chloro-N-((1r,4r)-4- methoxycyclohexyl)-2-(1-methyl-1H- imidazol-5-yl)pyrimidine-4- carboxamide |
| 330 | 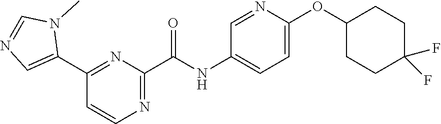 [SMILES: Cn1cncc1-c1ccnc(C(=O)Nc2ccc(OC3CCC(F)(F)CC3)nc2)n1] | N-(6-((4,4- difluorocyclohexyl)oxy)pyridin-3-yl)- 4-(1-methyl-1H-imidazol-5- yl)pyrimidine-2-carboxamide |
| 331 | 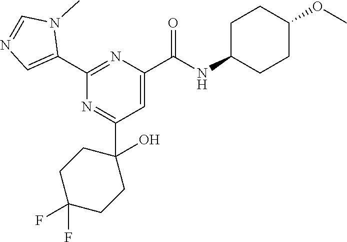 [SMILES: CO[C@H]1CC[C@H](NC(=O)c2cc(C3(O)CCC(F)(F)CC3)nc(-c3cncn3C)n2)CC1] | 6-(4,4-difluoro-1- hydroxycyclohexyl)-N-((1r,4r)-4- methoxycyclohexyl)-2-(1-methyl-1H- imidazol-5-yl)pyrimidine-4- carboxamide |
| 332 | 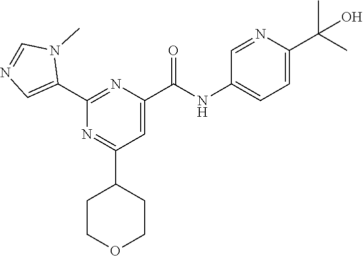 [SMILES: Cn1cncc1-c1nc(C(=O)Nc2ccc(C(C)(C)O)nc2)cc(C2CCOCC2)n1] | N-(6-(2-hydroxypropan-2-yl)pyridin- 3-yl)-2-(1-methyl-1H-imidazol-5-yl)- 6-(tetrahydro-2H-pyran-4- yl)pyrimidine-4-carboxamide |
| 333 | 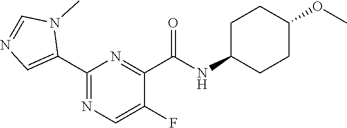 [SMILES: CO[C@H]1CC[C@H](NC(=O)c2nc(-c3cncn3C)ncc2F)CC1] | 5-fluoro-N-((1r,4r)-4- methoxycyclohexyl)-2-(1-methyl-1H- imidazol-5-yl)pyrimidine-4- carboxamide |
| 334 | 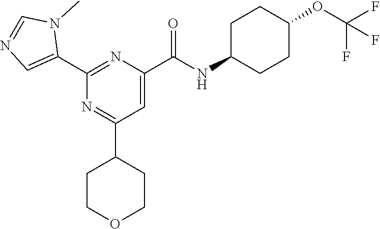 [SMILES: Cn1cncc1-c1nc(C(=O)N[C@H]2CC[C@H](OC(F)(F)F)CC2)cc(C2CCOCC2)n1] | 2-(1-methyl-1H-imidazol-5-yl)-6- (tetrahydro-2H-pyran-4-yl)-N- ((1r,4r)-4- (trifluoromethoxy)cyclohexyl)pyrimidine- 4-carboxamide |
| 335 | 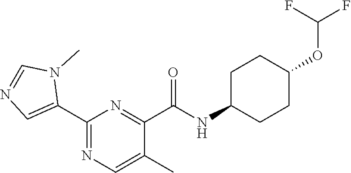 [SMILES: Cc1cnc(-c2cncn2C)nc1C(=O)N[C@H]1CC[C@H](OC(F)F)CC1] | N-((1r,4r)-4- (difluoromethoxy)cyclohexyl)-5- methyl-2-(1-methyl-1H-imidazol-5- yl)pyrimidine-4-carboxamide |
| 336 | 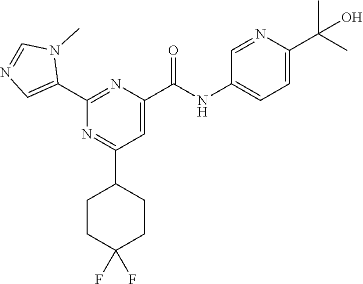 [SMILES: Cn1cncc1-c1nc(C(=O)Nc2ccc(C(C)(C)O)nc2)cc(C2CCC(F)(F)CC2)n1] | 6-(4,4-difluorocyclohexyl)-N-(6-(2- hydroxypropan-2-yl)pyridin-3-yl)-2- (1-methyl-1H-imidazol-5- yl)pyrimidine-4-carboxamide |
| 337 | 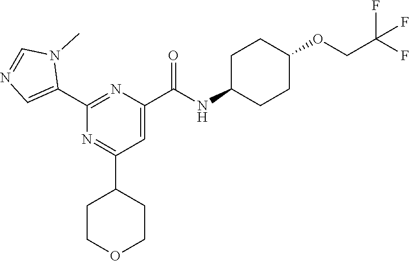 [SMILES: Cn1cncc1-c1nc(C(=O)N[C@H]2CC[C@H](OCC(F)(F)F)CC2)cc(C2CCOCC2)n1] | 2-(1-methyl-1H-imidazol-5-yl)-6- (tetrahydro-2H-pyran-4-yl)-N- ((1r,4r)-4-(2,2,2- trifluoroethoxy)cyclohexyl)pyrimidine- 4-carboxamide |
| 338 | 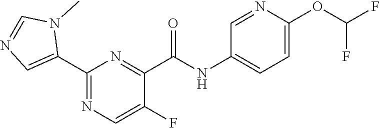 [SMILES: Cn1cncc1-c1ncc(F)c(C(=O)Nc2ccc(OC(F)F)nc2)n1] | N-(6-(difluoromethoxy)pyridin-3-yl)- 5-fluoro-2-(1-methyl-1H-imidazol-5- yl)pyrimidine-4-carboxamide |
| 339 | 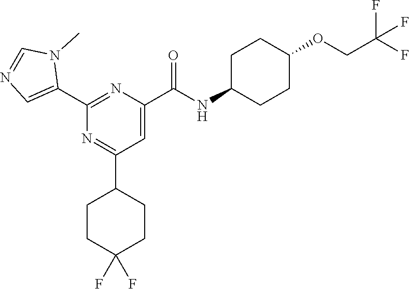 [SMILES: Cn1cncc1-c1nc(C(=O)N[C@H]2CC[C@H](OCC(F)(F)F)CC2)cc(C2CCC(F)(F)CC2)n1] | 6-(4,4-difluorocyclohexyl)-2-(1- methyl-1H-imidazol-5-yl)-N-((1r,4r)- 4-(2,2,2- trifluoroethoxy)cyclohexyl)pyrimidine- 4-carboxamide |
| 340 | 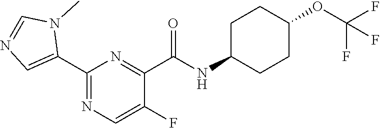 [SMILES: Cn1cncc1-c1ncc(F)c(C(=O)N[C@H]2CC[C@H](OC(F)(F)F)CC2)n1] | 5-fluoro-2-(1-methyl-1H-imidazol-5- yl)-N-((1r,4r)-4- (trifluoromethoxy)cyclohexyl)pyrimidine- 4-carboxamide |
| 341 | 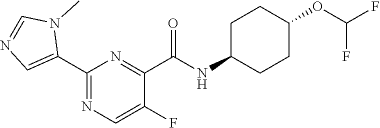 [SMILES: Cn1cncc1-c1ncc(F)c(C(=O)N[C@H]2CC[C@H](OC(F)F)CC2)n1] | N-((1r,4r)-4- (difluoromethoxy)cyclohexyl)-5- fluoro-2-(1-methyl-1H-imidazol-5- yl)pyrimidine-4-carboxamide |
| 342 | 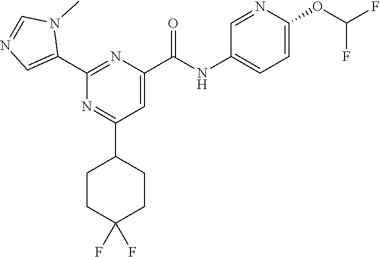 | 6-(4,4-difluorocyclohexyl)-N-(6- (difluoromethoxy)pyridin-3-yl)-2-(1- methyl-1H-imidazol-5-yl)pyrimidine- 4-carboxamide |
| 343 | 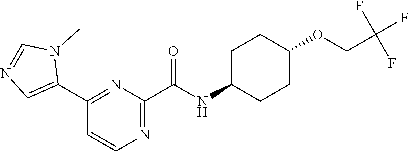 [SMILES: Cn1cncc1-c1ccnc(C(=O)N[C@H]2CC[C@H](OCC(F)(F)F)CC2)n1] | 4-(1-methyl-1H-imidazol-5-yl)-N- ((1r,4r)-4-(2,2,2- trifluoroethoxy)cyclohexyl)pyrimidine- 2-carboxamide |
| 344 | 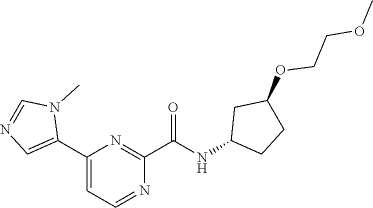 [SMILES: COCCO[C@H]1CC[C@H](NC(=O)c2nccc(-c3cncn3C)n2)C1] | N-((1S,3S)-3-(2- methoxyethoxy)cyclopentyl)-4-(1- methyl-1H-imidazol-5-yl)pyrimidine- 2-carboxamide |
| 345 | 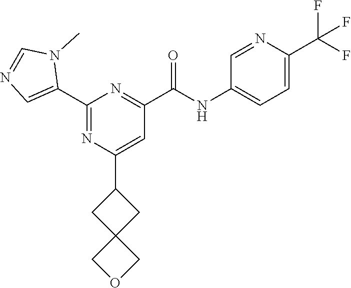 [SMILES: Cn1cncc1-c1nc(C(=O)Nc2ccc(C(F)(F)F)nc2)cc(C2CC3(COC3)C2)n1] | 2-(1-methyl-1H-imidazol-5-yl)-6-(2- oxaspiro[3.3]heptan-6-yl)-N-(6- (trifluoromethyl)pyridin-3- yl)pyrimidine-4-carboxamide |
| 346 | 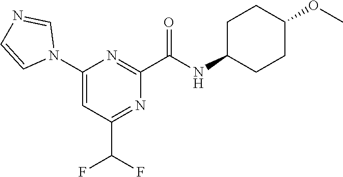 [SMILES: CO[C@H]1CC[C@H](NC(=O)c2nc(C(F)F)cc(-n3ccnc3)n2)CC1] | 4-(difluoromethyl)-6-(1H-imidazol-1- yl)-N-((1r,4r)-4- methoxycyclohexyl)pyrimidine-2- carboxamide |
| 347 | 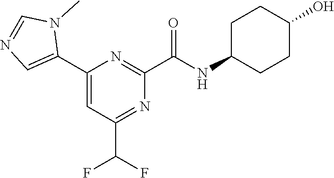 [SMILES: Cn1cncc1-c1cc(C(F)F)nc(C(=O)N[C@H]2CC[C@H](O)CC2)n1] | 4-(difluoromethyl)-N-((1r,4r)-4- hydroxycyclohexyl)-6-(1-methyl-1H- imidazol-5-yl)pyrimidine-2- carboxamide |
| 348 | 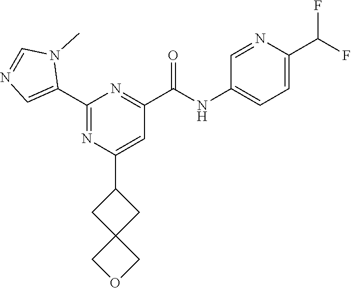 [SMILES: Cn1cncc1-c1nc(C(=O)Nc2ccc(C(F)F)nc2)cc(C2CC3(COC3)C2)n1] | N-(6-(difluoromethyl)pyridin-3-yl)-2- (1-methyl-1H-imidazol-5-yl)-6-(2- oxaspiro[3.3]heptan-6-yl)pyrimidine- 4-carboxamide |
| 349 | 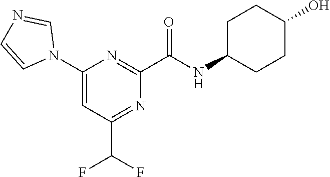 [SMILES: O=C(N[C@H]1CC[C@H](O)CC1)c1nc(C(F)F)cc(-n2ccnc2)n1] | 4-(difluoromethyl)-N-((1r,4r)-4- hydroxycyclohexyl)-6-(1H-imidazol- 1-yl)pyrimidine-2-carboxamide |
| 350 | 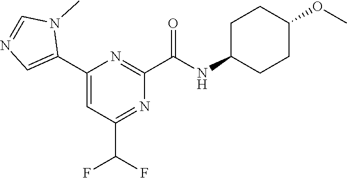 [SMILES: CO[C@H]1CC[C@H](NC(=O)c2nc(-c3cncn3C)cc(C(F)F)n2)CC1] | 4-(difluoromethyl)-N-((1r,4r)-4- methoxycyclohexyl)-6-(1-methyl-1H- imidazol-5-yl)pyrimidine-2- carboxamide |
| 351 | 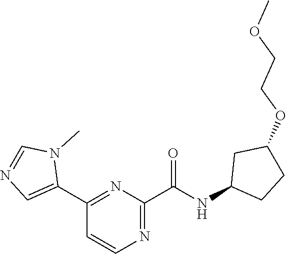 [SMILES: COCCO[C@@H]1CC[C@@H](NC(=O)c2nccc(-c3cncn3C)n2)C1] | N-((1R,3R)-3-(2- methoxyethoxy)cyclopentyl)-4-(1- methyl-1H-imidazol-5-yl)pyrimidine- 2-carboxamide |
| 352 | 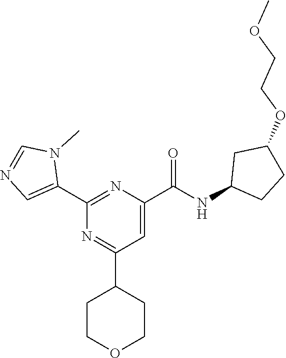 [SMILES: COCCO[C@@H]1CC[C@@H](NC(=O)c2cc(C3CCOCC3)nc(-c3cncn3C)n2)C1] | N-((1R,3R)-3-(2- methoxyethoxy)cyclopentyl)-2-(1- methyl-1H-imidazol-5-yl)-6- (tetrahydro-2H-pyran-4- yl)pyrimidine-4-carboxamide |
| 353 | 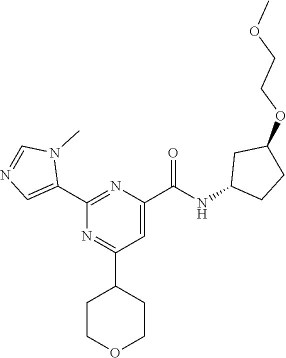 [SMILES: COCCO[C@H]1CC[C@H](NC(=O)c2cc(C3CCOCC3)nc(-c3cncn3C)n2)C1] | N-((1S,3S)-3-(2- methoxyethoxy)cyclopentyl)-2-(1- methyl-1H-imidazol-5-yl)-6- (tetrahydro-2H-pyran-4- yl)pyrimidine-4-carboxamide |
| 354 | 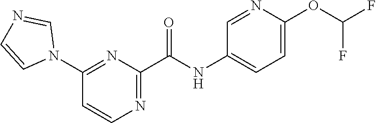 [SMILES: O=C(Nc1ccc(OC(F)F)nc1)c1nccc(-n2ccnc2)n1] | N-(6-(difluoromethoxy)pyridin-3-yl)- 4-(1H-imidazol-1-yl)pyrimidine-2- carboxamide |
| 355 | 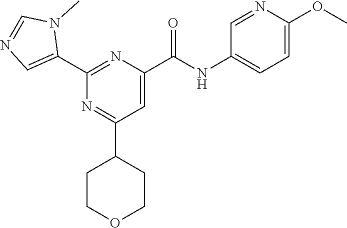 [SMILES: COc1ccc(NC(=O)c2cc(C3CCOCC3)nc(-c3cncn3C)n2)cn1] | N-(6-methoxypyridin-3-yl)-2-(1- methyl-1H-imidazol-5-yl)-6- (tetrahydro-2H-pyran-4- yl)pyrimidine-4-carboxamide |
| 356 | 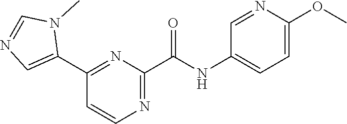 [SMILES: COc1ccc(NC(=O)c2nccc(-c3cncn3C)n2)cn1] | N-(6-methoxypyridin-3-yl)-4-(1- methyl-1H-imidazol-5-yl)pyrimidine- 2-carboxamide |
| 357 | 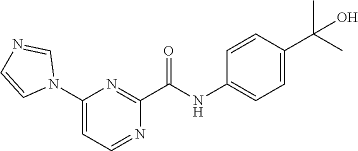 [SMILES: CC(C)(O)c1ccc(NC(=O)c2nccc(-n3ccnc3)n2)cc1] | N-(4-(2-hydroxypropan-2-yl)phenyl)-4-(1H- imidazol-1-yl)pyrimidine-2-carboxamide |
| 358 | 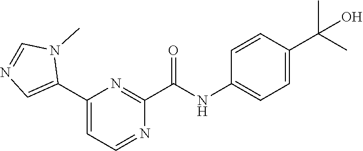 [SMILES: Cn1cncc1-c1ccnc(C(=O)Nc2ccc(C(C)(C)O)cc2)n1] | N-(4-(2-hydroxypropan-2-yl)phenyl)-4-(1- methyl-1H-imidazol-5-yl)pyrimidine-2- carboxamide |

In some embodiments, provided herein is a compound of formula (I), or any variation thereof, or a pharmaceutically acceptable salt of any of the foregoing, selected from the group consisting of:
4-(1H-imidazol-1-yl)-N-(pyridin-3-yl)picolinamide;
4-(1H-imidazol-1-yl)-N-(6-methylpyridin-3-yl)picolinamide;
N-(5-fluoropyridin-3-yl)-4-(1H-imidazol-1-yl)picolinamide;
4-(1H-imidazol-1-yl)-N-(pyridin-2-yl)picolinamide;
4-(1H-imidazol-1-yl)-N-phenylpicolinamide;
4-(1H-imidazol-1-yl)-N-(6-(trifluoromethyl)pyridin-3-yl)picolinamide;
4-(1H-imidazol-1-yl)-N-(pyridin-4-yl)picolinamide;
6-(1H-imidazol-1-yl)-N-(pyridin-3-yl)picolinamide;
3-(1H-imidazol-1-yl)-N-(pyridin-3-yl)benzamide;
2-(1H-imidazol-1-yl)-N-(pyridin-3-yl)isonicotinamide;
N-(pyridin-3-yl)-6-(thiazol-5-yl)picolinamide;
6-(1H-imidazol-1-yl)-N-(tetrahydro-2H-pyran-4-yl)picolinamide;
6-(1H-imidazol-1-yl)-N-(6-(trifluoromethyl)pyridin-3-yl)picolinamide;
6-(1H-imidazol-1-yl)-N-(4-(2-methoxyethoxy)cyclohexyl)picolinamide;
N-(3-hydroxybicyclo[1.1.1]pentan-1-yl)-6-(1H-imidazol-1-yl)picolinamide;
N-(bicyclo[1.1.1]pentan-1-yl)-6-(1H-imidazol-1-yl)picolinamide;
N-(2-fluorophenyl)-6-(1H-imidazol-1-yl)picolinamide;
N-(3-fluorophenyl)-6-(1H-imidazol-1-yl)picolinamide;
N-(4-fluorophenyl)-6-(1H-imidazol-1-yl)picolinamide;
6-(1H-imidazol-1-yl)-N-(6-methylpyridin-3-yl)picolinamide;
6-(1H-imidazol-1-yl)-N-(2-methylpyridin-4-yl)picolinamide;
6-(1H-imidazol-1-yl)-5-methyl-N-(pyridin-3-yl)picolinamide;
6-(1H-imidazol-1-yl)-N-(3-methoxycyclobutyl)picolinamide;
N-(2,4-difluorophenyl)-6-(1H-imidazol-1-yl)picolinamide;
6-(1H-imidazol-1-yl)-N-(6-methoxypyridin-3-yl)picolinamide;
6-(1H-imidazol-1-yl)-3-methyl-N-(pyridin-3-yl)picolinamide;
6-(1H-imidazol-1-yl)-N-(2-methylpyridin-3-yl)picolinamide;
6-(1H-imidazol-1-yl)-N-(4-methoxycyclohexyl)picolinamide;
6-(1H-imidazol-1-yl)-N-(4-methyl-6-(trifluoromethyl)pyridin-3-yl)picolinamide;
N-(4-hydroxycyclohexyl)-6-(1H-imidazol-1-yl)picolinamide;
6-(1H-imidazol-1-yl)-4-methyl-N-(pyridin-3-yl)picolinamide;
6-(1H-imidazol-1-yl)-N-(6-methylpyridin-2-yl)picolinamide;
N-(3,4-difluorophenyl)-6-(1H-imidazol-1-yl)picolinamide;
4-methyl-N-(pyridin-3-yl)-6-(thiazol-5-yl)picolinamide;
4-(1H-imidazol-1-yl)-N-(pyridin-3-yl)pyrimidine-2-carboxamide;
2-(1H-imidazol-1-yl)-N-(pyridin-3-yl)pyrimidine-4-carboxamide;
6-(1H-imidazol-1-yl)-N-(2-(trifluoromethyl)pyridin-4-yl)picolinamide;
N-(2-(difluoromethyl)pyridin-4-yl)-6-(1H-imidazol-1-yl)picolinamide;
6-(1H-imidazol-1-yl)-N-(pyridin-3-yl)-4-(trifluoromethyl)picolinamide;
N-(pyridin-3-yl)-6-(thiazol-5-yl)-4-(trifluoromethyl)picolinamide;
3-fluoro-6-(1H-imidazol-1-yl)-N-(6-(trifluoromethyl)pyridin-3-yl)picolinamide;
3-fluoro-6-(1H-imidazol-1-yl)-N-(4-(2-methoxyethoxy)cyclohexyl)picolinamide;
N-(1-acetylpiperidin-4-yl)-6-(1H-imidazol-1-yl)picolinamide;
methyl 4-(6-(1H-imidazol-1-yl)picolinamido)piperidine-1-carboxylate;
N-(2-acetyl-2-azaspiro[3.3]heptan-6-yl)-6-(1H-imidazol-1-yl)picolinamide;
methyl 6-(6-(1H-imidazol-1-yl)picolinamido)-2-azaspiro[3.3]heptane-2-carboxylate;
6-(1H-imidazol-1-yl)-N-(tetrahydro-2H-pyran-3-yl)picolinamide;
6-(thiazol-5-yl)-N-(6-(trifluoromethyl)pyridin-3-yl)picolinamide;
N-(6-methylpyridin-3-yl)-6-(thiazol-5-yl)picolinamide;
N-(2-methylpyridin-4-yl)-6-(thiazol-5-yl)picolinamide;
5-fluoro-6-(1H-imidazol-1-yl)-N-(6-(trifluoromethyl)pyridin-3-yl)picolinamide;
5-fluoro-6-(1H-imidazol-1-yl)-N-(4-(2-methoxyethoxy)cyclohexyl)picolinamide;
6-(1H-imidazol-1-yl)-N-(6-(trifluoromethyl)pyridin-3-yl)pyrazine-2-carboxamide;
6-(1H-imidazol-1-yl)-N-(pyridin-3-yl)pyrazine-2-carboxamide;
6-(1H-imidazol-1-yl)-N-(3-methoxycyclopentyl)picolinamide;
6-(1H-imidazol-1-yl)-N-(3-(4-(trifluoromethyl)phenyl)oxetan-3-yl)picolinamide;
6-(1-methyl-1H-imidazol-5-yl)-N-(pyridin-3-yl)picolinamide;
6-(1H-imidazol-1-yl)-N-(4-methoxycyclohexyl)-4-(trifluoromethyl)picolinamide;
6-(1H-imidazol-1-yl)-N-(4-(2-methoxyethoxy)cyclohexyl)-4-(trifluoromethyl)picolinamide;
N-(4-hydroxycyclohexyl)-6-(1H-imidazol-1-yl)-4-(trifluoromethyl)picolinamide;
6-(1H-imidazol-1-yl)-N-(tetrahydrofuran-3-yl)picolinamide;
N-(4-(difluoromethyl)cyclohexyl)-6-(1H-imidazol-1-yl)picolinamide;
N-(4-(2-methoxyethoxy)cyclohexyl)-6-(1-methyl-1H-imidazol-5-yl)picolinamide;
6-(5-carbamoyl-1H-imidazol-1-yl)-N-(6-(trifluoromethyl)pyridin-3-yl)picolinamide;
2-(1H-imidazol-1-yl)-N-(6-(trifluoromethyl)pyridin-3-yl)pyrimidine-4-carboxamide;
N-(4-(difluoromethyl)cyclohexyl)-2-(1H-imidazol-1-yl)pyrimidine-4-carboxamide;
2-(1-methyl-1H-imidazol-5-yl)-N-(6-(trifluoromethyl)pyridin-3-yl)pyrimidine-4-carboxamide;
N-(4-(difluoromethyl)cyclohexyl)-2-(1-methyl-1H-imidazol-5-yl)pyrimidine-4-carboxamide;
6-(1-methyl-1H-imidazol-5-yl)-N-(6-(trifluoromethyl)pyridin-3-yl)picolinamide;
4-(1H-imidazol-1-yl)-N-(6-(trifluoromethyl)pyridin-3-yl)pyrimidine-2-carboxamide;
N-(4-(difluoromethyl)cyclohexyl)-4-(1H-imidazol-1-yl)pyrimidine-2-carboxamide;
4-(1-methyl-1H-imidazol-5-yl)-N-(6-(trifluoromethyl)pyridin-3-yl)pyrimidine-2-carboxamide;
N-(4-(difluoromethyl)cyclohexyl)-4-(1-methyl-1H-imidazol-5-yl)pyrimidine-2-carboxamide;
6-(4-carbamoyl-1H-imidazol-1-yl)-N-(6-(trifluoromethyl)pyridin-3-yl)picolinamide;
N-(4-(difluoromethyl)cyclohexyl)-6-(1-methyl-1H-imidazol-5-yl)picolinamide;
N-(6,6-difluorospiro[3.3]heptan-2-yl)-2-(1H-imidazol-1-yl)pyrimidine-4-carboxamide;
2-(1H-imidazol-1-yl)-N-(4-(trifluoromethyl)cyclohexyl)pyrimidine-4-carboxamide;
2-(1H-imidazol-1-yl)-N-(4-methoxycyclohexyl)pyrimidine-4-carboxamide;
2-(1H-imidazol-1-yl)-N-(4-methylcyclohexyl)pyrimidine-4-carboxamide;
N-(6,6-difluorospiro[3.3]heptan-2-yl)-2-(1-methyl-1H-imidazol-5-yl)pyrimidine-4-carboxamide;
2-(1-methyl-1H-imidazol-5-yl)-N-(4-(trifluoromethyl)cyclohexyl)pyrimidine-4-carboxamide;
N-(4-methoxycyclohexyl)-2-(1-methyl-1H-imidazol-5-yl)pyrimidine-4-carboxamide;
2-(1-methyl-1H-imidazol-5-yl)-N-(4-methylcyclohexyl)pyrimidine-4-carboxamide;
2-(thiazol-5-ye-N-(6-(trifluoromethyl)pyridin-3-yl)pyrimidine-4-carboxamide;
N-(4-(difluoromethyl)cyclohexyl)-2-(thiazol-5-yl)pyrimidine-4-carboxamide;
6-(1H-imidazol-1-yl)-N-(4-methoxycyclohexyl)pyrazine-2-carboxamide;
N-(4-(difluoromethyl)cyclohexyl)-6-(1H-imidazol-1-yl)pyrazine-2-carboxamide;
6-(1-methyl-1H-imidazol-5-yl)-N-(6-(trifluoromethyl)pyridin-3-yl)pyrazine-2-carboxamide;
N-(4-methoxycyclohexyl)-6-(1-methyl-1H-imidazol-5-yl)pyrazine-2-carboxamide;
N-(4-(difluoromethyl)cyclohexyl)-6-(1-methyl-1H-imidazol-5-yl)pyrazine-2-carboxamide;
N-(6-(difluoromethyl)pyridin-3-yl)-2-(1H-imidazol-1-yl)pyrimidine-4-carboxamide;
N-(6-(difluoromethyl)pyridin-3-yl)-2-(1-methyl-1H-imidazol-5-yl)pyrimidine-4-carboxamide;
N-(6-(difluoromethyl)pyridin-3-yl)-6-(1H-imidazol-1-yl)picolinamide;
6-(1H-imidazol-1-yl)-N-(4-methoxycyclohexyl)-4-methylpicolinamide;
N-(4-(difluoromethyl)cyclohexyl)-6-(1H-imidazol-1-yl)-4-methylpicolinamide;
N-(4-methoxycyclohexyl)-4-methyl-6-(1-methyl-1H-imidazol-5-yl)picolinamide;
N-(4-(difluoromethyl)cyclohexyl)-4-methyl-6-(1-methyl-1H-imidazol-5-yl)picolinamide;
6-(1H-imidazol-1-yl)-N-(4-methoxycyclohexyl)-3-methylpicolinamide;
N-(4-(difluoromethyl)cyclohexyl)-6-(1H-imidazol-1-yl)-3-methylpicolinamide;
6-(1H-imidazol-1-yl)-N-(4-methylcyclohexyl)pyrazine-2-carboxamide;
N-(6,6-difluorospiro[3.3]heptan-2-yl)-6-(1H-imidazol-1-yl)pyrazine-2-carboxamide;
6-(1-methyl-1H-imidazol-5-yl)-N-(4-methylcyclohexyl)pyrazine-2-carboxamide;
N-(6,6-difluorospiro[3.3]heptan-2-yl)-6-(1-methyl-1H-imidazol-5-yl)pyrazine-2-carboxamide;
methyl 4-(2-(1H-imidazol-1-yl)pyrimidine-4-carboxamido)cyclohexane-1-carboxylate;
methyl 4-(2-(1-methyl-1H-imidazol-5-yl)pyrimidine-4-carboxamido)cyclohexane-1-carboxylate;
6-cyclopropyl-N-(4-methoxycyclohexyl)-2-(thiazol-5-yl)pyrimidine-4-carboxamide;
6-cyclopropyl-2-(thiazol-5-yl)-N-(6-(trifluoromethyl)pyridin-3-yl)pyrimidine-4-carboxamide;
6-cyclopropyl-N-(6-(difluoromethyl)pyridin-3-yl)-2-(thiazol-5-yl)pyrimidine-4-carboxamide;
2-(1H-imidazol-1-yl)-N-(4-methoxycyclohexyl)-6-(trifluoromethyl)pyrimidine-4-carboxamide;
N-(4-methylcyclohexyl)-2-(thiazol-5-yl)pyrimidine-4-carboxamide;
N-(4-methoxycyclohexyl)-2-(thiazol-5-yl)pyrimidine-4-carboxamide;
N-(6-(difluoromethyl)pyridin-3-yl)-2-(thiazol-5-yl)pyrimidine-4-carboxamide;
2-(thiazol-5-yl)-6-(trifluoromethyl)-N-(6-(trifluoromethyl)pyridin-3-yl)pyrimidine-4-carboxamide;
N-(6-(difluoromethyl)pyridin-3-yl)-2-(thiazol-5-yl)-6-(trifluoromethyl)pyrimidine-4-carboxamide;
2-(1H-imidazol-1-yl)-6-methyl-N-(6-(trifluoromethyl)pyridin-3-yl)pyrimidine-4-carboxamide;
2-(1H-imidazol-1-yl)-N-(4-methoxycyclohexyl)-6-methylpyrimidine-4-carboxamide;
2-(1H-imidazol-1-yl)-6-methyl-N-(4-methylcyclohexyl)pyrimidine-4-carboxamide;
N-(4-(difluoromethyl)cyclohexyl)-2-(1H-imidazol-1-yl)-6-methylpyrimidine-4-carboxamide;
N-(4-methoxycyclohexyl)-6-methyl-2-(1-methyl-1H-imidazol-5-yl)pyrimidine-4-carboxamide;
N-(4-(difluoromethyl)cyclohexyl)-6-methyl-2-(1-methyl-1H-imidazol-5-yl)pyrimidine-4-carboxamide;
6-methyl-2-(1-methyl-1H-imidazol-5-yl)-N-(4-methylcyclohexyl)pyrimidine-4-carboxamide;
N-(6,6-difluorospiro[3.3]heptan-2-yl)-6-methyl-2-(1-methyl-1H-imidazol-5-yl)pyrimidine-4-carboxamide;
2-(1H-imidazol-1-yl)-6-methoxy-N-(4-methoxycyclohexyl)pyrimidine-4-carboxamide;
6-hydroxy-2-(1H-imidazol-1-yl)-N-(4-methoxycyclohexyl)pyrimidine-4-carboxamide;
N-(6-(difluoromethyl)pyridin-3-yl)-2-(1H-imidazol-1-yl)-6-methoxypyrimidine-4-carboxamide;
6-methoxy-N-(4-methoxycyclohexyl)-2-(1-methyl-1H-imidazol-5-yl)pyrimidine-4-carboxamide;
N-(6-(difluoromethyl)pyridin-3-yl)-6-methoxy-2-(1-methyl-1H-imidazol-5-yl)pyrimidine carboxamide;
6-methoxy-N-(4-methoxycyclohexyl)-2-(thiazol-5-yl)pyrimidine-4-carboxamide;
N-(6-(difluoromethyl)pyridin-3-yl)-6-methoxy-2-(thiazol-5-yl)pyrimidine-4-carboxamide;
N-(6-(difluoromethyl)pyridin-3-yl)-4-methyl-6-(thiazol-5-yl)picolinamide;
N-(4-methoxycyclohexyl)-4-methyl-6-(thiazol-5-yl)picolinamide;
N-(4-(2-hydroxypropan-2-yl)cyclohexyl)-6-(1H-imidazol-1-yl)picolinamide;
N-(6,6-difluorobicyclo[3.1.0]hexan-3-yl)-6-(1H-imidazol-1-yl)picolinamide;
N-(4,4-difluorocyclohexyl)-6-(1H-imidazol-1-yl)picolinamide;
2-(1H-imidazol-1-yl)-N-(6-methylpyridin-3-yl)-6-(trifluoromethyl)pyrimidine-4-carboxamide;
N-(4-(difluoromethyl)cyclohexyl)-2-(1H-imidazol-1-yl)-6-(trifluoromethyl)pyrimidine-4-carboxamide;
N-(4-(methoxymethyl)cyclohexyl)-2-(thiazol-5-yl)pyrimidine-4-carboxamide;
N-(4-(hydroxymethyl)cyclohexyl)-2-(thiazol-5-yl)pyrimidine-4-carboxamide;
6-cyclopropyl-N-(4-(hydroxymethyl)cyclohexyl)-2-(thiazol-5-yl)pyrimidine-4-carboxamide;
6-cyclopropyl-N-(1,1-dioxidotetrahydro-2H-thiopyran-4-yl)-2-(thiazol-5-yl)pyrimidine-4-carboxamide;
6-(4H-1,2,4-triazol-4-yl)-N-(6-(trifluoromethyl)pyridin-3-yl)picolinamide;
N-(6-(difluoromethyl)pyridin-3-yl)-4-(1H-imidazol-1-yl)pyrimidine-2-carboxamide;
4-(1H-imidazol-1-yl)-N-(4-methoxycyclohexyl)pyrimidine-2-carboxamide;
4-(1H-imidazol-1-yl)-N-(4-methylcyclohexyl)pyrimidine-2-carboxamide;
N-(6-(difluoromethyl)pyridin-3-yl)-4-(1-methyl-1H-imidazol-5-yl)pyrimidine-2-carboxamide;
N-(4-methoxycyclohexyl)-4-(1-methyl-1H-imidazol-5-yl)pyrimidine-2-carboxamide;
4-(1-methyl-1H-imidazol-5-yl)-N-(4-methylcyclohexyl)pyrimidine-2-carboxamide;
N-(6-(difluoromethyl)pyridin-3-yl)-4-(thiazol-5-yl)pyrimidine-2-carboxamide;
N-(4-methoxycyclohexyl)-2-(1-methyl-1H-imidazol-5-yl)-6-(trifluoromethyl)pyrimidine-4-carboxamide;
N-(4-(difluoromethyl)cyclohexyl)-2-(1-methyl-1H-imidazol-5-yl)-6-(trifluoromethyl)pyrimidine-4-carboxamide;
2-(1-methyl-1H-imidazol-5-yl)-N-(4-methylcyclohexyl)-6-(trifluoromethyl)pyrimidine carboxamide;
N-(6,6-difluorospiro[3.3]heptan-2-yl)-2-(1-methyl-1H-imidazol-5-yl) (trifluoromethyl)pyrimidine-4-carboxamide;
N-(4-(2-methoxyethoxy)cyclohexyl)-6-methyl-2-(1-methyl-1H-imidazol-5-yl)pyrimidine carboxamide;
2-(1H-imidazol-1-yl)-N-(4-(2-methoxyethoxy)cyclohexyl)-6-methylpyrimidine-4-carboxamide;
2-(1H-imidazol-1-yl)-N-(4-(2-methoxyethoxy)cyclohexyl)-6-(trifluoromethyl)pyrimidine-4-carboxamide;
N-(6-(difluoromethyl)pyridin-3-yl)-6-(2-hydroxypropan-2-yl)-2-(1H-imidazol-1-yl)pyrimidine-4-carboxamide;
N-(6-(difluoromethyl)pyridin-3-yl)-6-(2-hydroxypropan-2-yl)-2-(1-methyl-1H-imidazol-5-yl)pyrimidine-4-carboxamide;
6-cyclopropyl-2-(1H-imidazol-1-yl)-N-(4-methoxycyclohexyl)pyrimidine-4-carboxamide;
6-cyclopropyl-N-(4-(difluoromethyl)cyclohexyl)-2-(1H-imidazol-1-yl)pyrimidine-4-carboxamide;
6-cyclopropyl-N-(6-(difluoromethyl)pyridin-3-yl)-2-(1H-imidazol-1-yl)pyrimidine-4-carboxamide;
N-(6-(difluoromethyl)pyridin-3-yl)-4-methoxy-6-(thiazol-5-yl)picolinamide;
2-(1H-imidazol-1-yl)-N-(4-methylcyclohexyl)-6-(trifluoromethyl)pyrimidine-4-carboxamide;
N-(4-(2-methoxyethoxy)cyclohexyl)-2-(1-methyl-1H-imidazol-5-yl)-6-(trifluoromethyl)pyrimidine-4-carboxamide;
6-(1H-imidazol-1-yl)-4-methoxy-N-(4-methylcyclohexyl)picolinamide;
2-(1H-imidazol-1-yl)-6-isopropyl-N-(4-methoxycyclohexyl)pyrimidine-4-carboxamide;
N-(4-(difluoromethyl)cyclohexyl)-2-(1H-imidazol-1-yl)-6-isopropylpyrimidine-4-carboxamide;
N-(6-(difluoromethyl)pyridin-3-yl)-2-(1H-imidazol-1-yl)-6-isopropylpyrimidine-4-carboxamide;
2-(1H-imidazol-1-yl)-6-isopropyl-N-(6-(trifluoromethyl)pyridin-3-yl)pyrimidine-4-carboxamide;
6-isopropyl-N-(4-methoxycyclohexyl)-2-(1-methyl-1H-imidazol-5-yl)pyrimidine-4-carboxamide;
N-(4-(difluoromethyl)cyclohexyl)-6-isopropyl-2-(1-methyl-1H-imidazol-5-yl)pyrimidine carboxamide;
N-(6-(difluoromethyl)pyridin-3-yl)-6-isopropyl-2-(1-methyl-1H-imidazol-5-yl)pyrimidine carboxamide;
6-isopropyl-2-(1-methyl-1H-imidazol-5-yl)-N-(6-(trifluoromethyl)pyridin-3-yl)pyrimidine carboxamide;
6-(2-hydroxypropan-2-yl)-2-(1H-imidazol-1-yl)-N-(6-(trifluoromethyl)pyridin-3-yl)pyrimidine-4-carboxamide;
6-(2-hydroxypropan-2-yl)-2-(1-methyl-1H-imidazol-5-yl)-N-(6-(trifluoromethyl)pyridin-3-yl)pyrimidine-4-carboxamide;
N-(4-(difluoromethyl)cyclohexyl)-6-(2-hydroxypropan-2-yl)-2-(1-methyl-1H-imidazol-5-yl)pyrimidine-4-carboxamide;
6-(2-hydroxypropan-2-yl)-N-(4-methoxycyclohexyl)-2-(1-methyl-1H-imidazol-5-yl)pyrimidine-4-carboxamide;
N-(6-(difluoromethyl)pyridin-3-yl)-4-(pyrrolidin-1-yl)-6-(thiazol-5-yl)picolinamide;
6-(1H-imidazol-1-yl)-N-(4-methoxycyclohexyl)-4-(pyrrolidin-1-yl)picolinamide;
6-cyclobutyl-N-(4-methoxycyclohexyl)-2-(1-methyl-1H-imidazol-2-yl)pyrimidine-4-carboxamide;
6-cyclobutyl-N-(4,4-difluorocyclohexyl)-2-(1-methyl-1H-imidazol-2-yl)pyrimidine-4-carboxamide;
6-cyclobutyl-N-(6-(difluoromethyl)pyridin-3-yl)-2-(1-methyl-1H-imidazol-2-yl)pyrimidine-4-carboxamide;
6-cyclobutyl-2-(1H-imidazol-1-yl)-N-(4-methoxycyclohexyl)pyrimidine-4-carboxamide;
6-cyclobutyl-N-(4,4-difluorocyclohexyl)-2-(1H-imidazol-1-yl)pyrimidine-4-carboxamide; and
6-cyclobutyl-N-(6-(difluoromethyl)pyridin-3-yl)-2-(1H-imidazol-1-yl)pyrimidine-4-carboxamide,
   or a stereoisomer or tautomer thereof, or a pharmaceutically acceptable salt of any of the foregoing.

In some variations, any of the compounds described herein, such as a compound of formula (I), or any variation thereof, or a compound of Table 1 may be deuterated (e.g., a hydrogen atom is replaced by a deuterium atom). In some of these variations, the compound is deuterated at a single site. In other variations, the compound is deuterated at multiple sites. Deuterated compounds can be prepared from deuterated starting materials in a manner similar to the preparation of the corresponding non-deuterated compounds. Hydrogen atoms may also be replaced with deuterium atoms using other method known in the art.

Any formula given herein, such as formula (I) (I-A), (I-A1), (I-A2), (I-B), (I-C), (I-D), (I-E), (I-F), (I-G), (I-H), or (I-J), is intended to represent compounds having structures depicted by the structural formula as well as certain variations or forms. In particular, compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Where a compound of Table 1 is depicted with a particular stereochemical configuration, also provided herein is any alternative stereochemical configuration of the compound, as well as a mixture of stereoisomers of the compound in any ratio. For example, where a compound of Table 1 has a stereocenter that is in an “S” stereochemical configuration, also provided herein is enantiomer of the compound wherein that stereocenter is in an “R” stereochemical configuration. Likewise, when a compound of Table 1 has a stereocenter that is in an “R” configuration, also provided herein is enantiomer of the compound in an “S” stereochemical configuration. Also provided are mixtures of the compound with both the “S” and the “R” stereochemical configuration. Additionally, if a compound of Table 1 has two or more stereocenters, also provided are any enantiomer or diastereomer of the compound. For example, if a compound of Table 1 contains a first stereocenter and a second stereocenter with “R” and “R” stereochemical configurations, respectively, also provided are stereoisomers of the compound having first and second stereocenters with “S” and “S” stereochemical configurations, respectively, “S” and “R” stereochemical configurations, respectively, and “R” and “S” stereochemical configurations, respectively. If a compound of Table 1 contains a first stereocenter and a second stereocenter with “S” and “S” stereochemical configurations, respectively, also provided are stereoisomers of the compound having first and second stereocenters with “R” and “R” stereochemical configurations, respectively, “S” and “R” stereochemical configurations, respectively, and “R” and “S” stereochemical configurations, respectively. If a compound of Table 1 contains a first stereocenter and a second stereocenter with “S” and “R” stereochemical configurations, respectively, also provided are stereoisomers of the compound having first and second stereocenters with “R” and “S” stereochemical configurations, respectively, “R” and “R” stereochemical configurations, respectively, and “S” and “S” stereochemical configurations, respectively. Similarly, if a compound of Table 1 contains a first stereocenter and a second stereocenter with “R” and “S” stereochemical configurations, respectively, also provided are stereoisomers of the compound having first and second stereocenters with “S” and “R” stereochemical configurations, respectively, “R” and “R” stereochemical configurations, respectively, and “S” and “S” stereochemical configurations, respectively. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Representative examples of compounds detailed herein, including intermediates and final compounds, are depicted in the tables and elsewhere herein. It is understood that in one aspect, any of the compounds may be used in the methods detailed herein, including, where applicable, intermediate compounds that may be isolated and administered to an individual or subject.

The compounds depicted herein may be present as salts even if salts are not depicted, and it is understood that the compositions and methods provided herein embrace all salts and solvates of the compounds depicted here, as well as the non-salt and non-solvate form of the compound, as is well understood by the skilled artisan. In some embodiments, the salts of the compounds provided herein are pharmaceutically acceptable salts.

In one variation, the compounds herein are synthetic compounds prepared for administration to an individual or subject. In another variation, compositions are provided containing a compound in substantially pure form. In another variation, provided are pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

Any variation or embodiment of

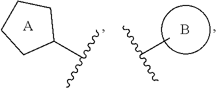

X¹, X², X³, X⁴, R^{a}, R^{b}, R^{x}, R^{y}, and R^{z} provided herein can be combined with every other variation or embodiment of

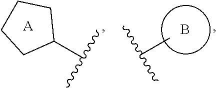

X¹, X², X³, X⁴, R^{a}, R^{b}, R^{x}, R^{y}, and R^{z}, the same as if each and every combination had been individually and specifically described.

Other embodiments will be apparent to those skilled in the art from the following detailed description.

As used herein, when any variable occurs more than one time in a chemical formula, its definition on each occurrence is independent of its definition at every other occurrence.

Formula (I) includes all subformulae thereof.

One of skilled in the art would understand that the compounds may be named or identified using various commonly recognized nomenclature systems and symbols. By way of example, the compounds may be named or identified with common names, systematic or non-systematic names. The nomenclature systems and symbols that are commonly recognized in the art of chemistry include, for example, Chemical Abstract Service (CAS), ChemBioDraw Ultra, and International Union of Pure and Applied Chemistry (IUPAC).

In some embodiments, the compounds of the disclosure, or a pharmaceutically acceptable salt thereof, may have advantages related to one or more of the following: hERG profile, toxicity profile, safety window, selectivity, off-target profile, drug/drug interaction risk, PK parameters including bioavailability, clearance and half life, mechanism of action, CYP inhibition and/or induction profile, permeability and/or efflux, solubility, metabolism, unbound fraction percentage, adequate human dose, and ease of synthesis on a large scale.

### Compositions

Also provided are compositions, such as pharmaceutical compositions that include a compound disclosed and/or described herein and one or more additional medicinal agents, pharmaceutical agents, adjuvants, carriers, excipients, and the like. Suitable medicinal and pharmaceutical agents include those described herein. In some embodiments, the pharmaceutical composition includes a pharmaceutically acceptable excipient or adjuvant and at least one chemical entity as described herein. Examples of pharmaceutically acceptable excipients include, but are not limited to, mannitol, lactose, starch, magnesium stearate, sodium saccharine, talcum, cellulose, sodium crosscarmellose, glucose, gelatin, sucrose, and magnesium carbonate. In some embodiments, provided are compositions, such as pharmaceutical compositions, that contain one or more compounds described herein, or a pharmaceutically acceptable salt thereof.

In some embodiments, provided is a pharmaceutically acceptable composition comprising an effective amount of a compound of formula (I), such as a compound of formula (I-A), (I-A1), (I-A2), (I-B), (I-C), (I-D), (I-E), (I-F), (I-G), (I-H), or (I-J), or a compound of Table 1, or a pharmaceutically acceptable salt thereof. In some aspects, a composition may contain a synthetic intermediate that may be used in the preparation of a compound described herein. The compositions described herein may contain any other suitable active or inactive agents.

Any of the compositions described herein may be sterile or contain components that are sterile. Sterilization can be achieved by methods known in the art. Any of the compositions described herein may contain one or more compounds or conjugates that are substantially pure.

Also provided are packaged pharmaceutical compositions, comprising a pharmaceutical composition as described herein and instructions for using the composition to treat a patient suffering from a disease or condition described herein.

### Methods of Use

Compounds and compositions detailed herein, such as a pharmaceutical composition comprising a compound of any formula provided herein, or a pharmaceutically acceptable salt thereof, and a pharmaceutically acceptable carrier or excipient, may be used in methods of administration and treatment as provided herein.

Without being bound by theory, the compounds and pharmaceutical compositions disclosed herein are believed to act by modulating CD38. In some embodiments, the compounds and pharmaceutical compositions disclosed herein are inhibitors of CD38. In some embodiments, provided are methods of treating a disease or condition mediated by CD38 activity in an individual or subject, comprising administering to the individual or subject in need thereof a compound of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof. In some embodiments, provided are methods of treating cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder in an individual or subject, comprising administering to the individual or subject in need thereof a compound of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof.

In some embodiments, provided are methods of preventing a disease or condition mediated by CD38 activity in an individual or subject, comprising administering to the individual or subject in need thereof a compound of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof. In some embodiments, provided are methods of preventing cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder in an individual or subject, comprising administering to the individual or subject in need thereof a compound of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof.

Also provided herein is the use of a compound of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof in the manufacture of a medicament for treatment of a disease or condition mediated by CD38 activity in a subject. In some aspects, provided is a compound or composition as described herein for use in a method of treatment of the human or animal body by therapy. In some embodiments, provided herein are compounds of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof, for use in a method of treatment of the human or animal body by therapy. In some embodiments, provided herein are compounds of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof, for use in treating a disease or condition mediated by CD38 activity. In some embodiments, the disease or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder.

Also provided herein is the use of a compound of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof in the manufacture of a medicament for prevention of a disease or condition mediated by CD38 activity in a subject. In some aspects, provided is a compound or composition as described herein for use in a method of prevention of the human or animal body by therapy. In some embodiments, provided herein are compounds of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof, for use in a method of prevention of the human or animal body by therapy. In some embodiments, provided herein are compounds of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof, for use in preventing a disease or condition mediated by CD38 activity. In some embodiments, the disease or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder.

Also provided herein are compositions (including pharmaceutical compositions) as described herein for the use in treating, preventing, and/or delaying the onset and/or development of a disease described herein and other methods described herein. In certain embodiments, the composition comprises a pharmaceutical formulation which is present in a unit dosage form.

In some embodiments, the subject is a mammal. In some embodiments, the subject is a mouse, rat, dog, cat, rabbit, pig, sheep, horse, cow, or human. In some embodiments, the subject is a human.

There are numerous conditions in which small molecule-mediated modulation of CD38 hydrolase activity would potentially be clinically beneficial (Chini et al, Trends Pharmacol Sci, 2018 April; 39(4):424-436, Hogan et al, Front. Immunol., 2019, Guerreiro et al, Cells. 2020 February; 9(2): 471, Peidra-Quintero et al, Front Immunol. 2020; 11: 597959, Kar et al, Cells, 2020 Jul. 17; 9(7):1716, Verdin, Science. 2015, 350(6265):1208-13). These conditions include, but are not limited to, cardiac diseases, chemotherapy induced tissue damage, inflammation, myocarditis, myocarditis associated with SARS-CoV-2 infection, immune-oncology, renal diseases, fibrotic diseases, metabolic diseases, muscular diseases, neurological diseases and injuries, diseases caused by impaired stem cell function, DNA damage and primary mitochondrial disorders, and ocular diseases. In some embodiments, the disease or condition mediated by CD38 activity is a cardiac disease, chemotherapy induced tissue damage, a renal disease, a metabolic disease, a fibrotic disease, an inflammatory disease, a muscular disease, a neurological disease or injury, a disease caused by immune-suppression by cancerous cells, a disease caused by impaired stem cell function, or DNA damage and primary mitochondrial disorder.

Cardiac diseases. In various preclinical models of heart failure, NAD levels are decreased with activation of CD38. In these models, cardiac function can be rescued, either by inhibiting the CD38 activity (Reyes et al, PNAS. 2015, 112:11648-53; Boslett et al., J Pharmacol Exp Ther. 2017; 361:99-108; Boslett et al., J Pharmacol Exp Ther. 2019; 369:55-64). Thus, blocking the catalytic activity of CD38 with a small molecule inhibitor is a promising strategy to treat various forms of heart failure. Additionally, with the increased expression and activity of CD38 with age, ageing-related arrhythmias, such as atrial fibrillation, also indicate the benefit of inhibiting CD38 activity to reduce atrial fibrillation (Lin et al, J Biol Chem. 2017; 292:13243-57).

Chemotherapy induced tissue damage. Use of chemotherapy regimens frequently is limited by toxicity to healthy tissues and severe oxidative stress is thought to play a major role. Triggering of CD38-dependent NAD (P)-decline has been shown to trigger a pathogenic response. Therefore, CD38 inhibitors are considered broadly useful in various settings of chemotherapy to prevent reversible and irreversible secondary pathologies. Examples are anthracycline and trastuzumab cardiotoxicity, cisplatin induced kidney injury, peripheral neuropathies induced by cisplatin, paclitaxel, vincristine and other agents.

Metabolic disease. CD38 inhibiting boosts improves insulin sensitivity, dyslipidemia, mitochondrial function in metabolic disease and protects from/improves non-alcoholic and alcoholic steatohepatitis in preclinical models. More than 3 million people per year in the U.S. alone are diagnosed with non-alcoholic steatohepatitis and it is one of the leading causes of liver transplantation. See Guarino and Dufour, Metabolites. 2019, Sep. 10; 9 (9), pii: E180; Yoshino et al., Cell Metab. 2011, 14(4):528-36.

Muscular diseases. Preclinical data has suggested that NAD+ boosting strategies could alleviate skeletal muscle dysfunction in a number of conditions, including Duchenne's muscular dystrophy, and age-related sarcopenia. See Zhang et al., Clin Sci (Lond). 2019, 133(13):1505-1521; Mohamed et al., Aging (Albany N.Y.). 2014, 6(10):820-34; Ryu et al., Sci Transl Med. 2016, 8(361):361ra139.

Neurological diseases and injuries. Inhibiting degradation of NAD by means of CD38 inhibition is neuroprotective and of therapeutic benefit in a wide range of preclinical models of neurological diseases and injuries, including age-related cognitive decline, glaucoma, ischemic stroke, and ALS. See Johnson et al., NPJ Aging Mech Dis. 2018, 4:10; Harlan et al., J Biol Chem. 2016, 291(20):10836-46; Zhao et al., Stroke. 2015, July; 46(7):1966-74; Williams et al., Front Neurosci. 2017, Apr. 25; 11:232.

Fibrotic diseases. Expression of CD38 in Multi-organ fibrosis such as scleroderma has been linked to disease severity, as observed in the skin in patients with SSc, as levels are associated with both clinical disease severity and profibrotic signaling activity. See Shi et al., iScience, 2021, 24, 101902. Nonalcoholic steatohepatitis (NASH) originating from obesity-mediated steatosis, facilitating inflammation and fibrosis also may be mediated by CD38 NAD-hydrolyzing activity. CD38 has been observed to be involved in high-fat diet (HFD)-mediated fatty liver. CD38-deficient mice are protected from steatosis (Barbosa et al, 2007, FASEB J., 21, 3629-3639.

Myocarditis. Myocarditis is an autoimmune disorder that can be caused by immune modulators such as immune checkpoint inhibitors, and viral infection such as coxsackie virus B3 (CVB3). Proinflammatory Th1 responses during myocarditis increase myocardial inflammation and can result in hemodynamic and energetic stress in the heart. Chronic inflammation and energetic stress can lead to reduced cardiac function, remodeling, and heart failure. CD38 is present on multiple cell types, contributes to pro-inflammatory Th1 phenotypes, and reduces cellular NAD+ pools, which can result in metabolic stress. Genomic studies have demonstrated that CD38 expression is increased during CVB3 myocarditis. CD38 inhibition will block pro-inflammatory responses, immune-checkpoint inhibitor-induced myocarditis maintain energetic homeostasis and reduce myocarditis severity.

Myocarditis and pericarditis associated with SARS-CoV-2 infection: CD38 plays a central role in altered immunometabolism resulting from COVID-19 infection responsible for the inflammatory disease of the myocardium. Covid-associated myocarditis can occur as acute or fulminant with severe manifestation associated with acute heart failure, cardiogenic shock and life-threatening arrhythmias and chronic, the latter being subclinical with long-term cardiovascular compilations.

Complementing Immune-checkpoint Inhibitors for Oncology: CD38 has a crucial role in driving exhaustion of T cells, which is refractory to the PD-1 mediated functional rejuvenation (Verma et al Nat. Immunol. 20 1231-1243, Chatterjee et al Cell Metabolism, 2018, 85-100, Wu et al, Cancer Immunology, Immunotherapy, 2021). CD38 inhibitors help to rejuvenate the T-cells and result in a better manifestation of the anti-tumor property of the tumor-infiltrating T cells.

Provided in some embodiments are methods of treating a disease or condition mediated by CD38 activity in a subject in need thereof, comprising administering to the individual or subject in need thereof a compound of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof, wherein the disease or condition is selected from the group consisting of cardiac diseases, chemotherapy induced tissue damage, renal diseases, metabolic diseases, muscular diseases, neurological diseases and injuries, diseases caused by impaired stem cell function, and DNA damage and primary mitochondrial disorders.

Additional applications of small molecule CD38 modulators are provided in Table 2.

TABLE 2

| | |
|---|---|
| Cancer and | Combination strategy with Immune |
| Chemotherapy | Checkpoint Therapy |
| induced tissue | Anthracycline and trastuzumab cardiotoxicity |
| damage | Proteasome inhibitor cardiotoxicity |
| | Cisplatin induced kidney injury |
| | Prevention/treatment of cognitive dysfunction resulting |
| | from chemotherapy (“chemo brain”) |
| | Chemotherapy induced impairment of hematopoiesis |
| | and myelosuppression |
| | Cachexia of cancer |
| Cardiovascular | Heart failure with reduced ejection fraction |
| diseases | Heart failure with preserved ejection fraction |
| | Hypertrophic cardiomyopathy |
| | Cardiac arrhythmias |
| | Duchenne Muscular Dystrophy-related cardiac |
| | dysfunction |
| | Cardiac dysfunction associated with Scleroderma, |
| | Lupus, Mitochondrial Disorders, Kawasaki Disease |
| | Hypertension |
| | Myocardial Infarction |
| | Genetic disorders that cause heart failure and life- |
| | threatening arrhythmia as in dilated cardiomyopathy |
| | (DCM), hypertrophic cardiomyopathy (HCM), |
| | arrhythmogenic cardiomyopathy (AC), and restrictive |
| | cardiomyopathy (RCM) |
| | Right ventricular hypertrophy (RVH) in response to |
| | pressure overload, most commonly due to severe lung |
| | disease and pulmonary hypertension |
| Renal diseases | Acute kidney injury including nephropathy following |
| | major surgeries including cardiac and vascular surgeries |
| | Acute kidney injury following hypotension, |
| | hemorrhagic shock, or cardiac arrest |
| | Acute kidney injury following exposure to contrast |
| | imaging agents used for MRI, CT scans, or other |
| | imaging modalities, particularly in the context of |
| | diabetes |
| | Chronic kidney disease |
| | Glomerular nephritis |
| | Kidney mesangial cell hypertrophy |
| | Arterial venous fistula maturation |
| Chronic | Chronic obstructive pulmonary disease |
| inflammatory | Asthma |
| and fibrotic | Scleroderma |
| diseases | Dermatomyositis |
| | Lupus erythematosus |
| | Rheumatoid arthritis and spondyloarthropathy |
| | Juvenile idiopathic arthritis |
| | Crohn's disease |
| | Inflammatory Bowel Disease |
| | Eczema |
| | Psoriasis and psoriatic arthritis |
| | Idiopathic pulmonary fibrosis |
| Vascular | Arterial and venous thrombosis |
| diseases | Ischemic Stroke |
| | Arteriosclerosis |
| Metabolic | Obesity |
| dysfunction | Diabetes |
| | Metabolic Syndrome |
| | Alcoholic steatohepatitis |
| | Non-alcoholic steatohepatitis |
| | Dyslipidemia |
| | Diabetic neuropathy |
| | Diabetic gastroparesis |
| Muscular | Muscular dystrophies, including: Duchenne, Becker's, |
| diseases | Congenital, Distal, Emery-Dreifuss', Facio-scapulo- |
| | humeral, Limb-girdle, myotonic, and oculopharyngeal |
| | Sarcopenia |
| | Frailty |
| | Polymyositis |
| | Muscle stem cell senescence developed in the context |
| | of nutritional deficiencies |
| | Non-mitochondrial myopathies such as inherited |
| | myopathies, myotonia, congenital myopathies selected |
| | from nemaline myopathy, multi/minicore myopathy, |
| | centronuclear myopathy and metabolic myopathies, |
| | inflammatory myopathies |
| Neurological | Depression |
| diseases | Frontotemporal dementia |
| and injuries | Multiple sclerosis |
| | Amyotrophic lateral sclerosis |
| | Peripheral neuropathy due to diabetes, chemotherapy |
| | Alzheimer's disease |
| | Parkinson's disease |
| | Huntington's Disease |
| | Spinal muscular atrophy |
| | Spinocerebellar ataxias |
| | Spastic paraplegias |
| | Glaucoma |
| | Age-related macular degeneration |
| | Age-related cognitive decline |
| | Noise induced and age-related hearing loss |
| | Ischemic stroke |
| | Traumatic brain injury |
| | Neonatal nerve damage |
| | Optic nerve injury |
| | Spinal cord injuries |
| | Peripheral neuropathies or tissue inflammation induced |
| | by cisplatin, paclitaxel, vincristine, other |
| | chemotherapeutic agents, or radiation. |
| | Peripheral neuropathies (length and non-length |
| | dependent) affecting motor, sensory, or autonomic |
| | nerves, arising from: diabetes, impaired glucose |
| | tolerance, hypertension, infection, trauma, autoimmune |
| | disorders, vasculitis, arteriosclerosis, vitamin |
| | deficiencies (particularly B6 and Bl2), alcoholism, liver |
| | or kidney disease, or exposure to toxins |
| DNA damage | Xeroderma pigmentosum |
| disorders and | Cockayne syndrome |
| Primary | Ataxia telangiectasia |
| Mitochondrial | MEGDEL syndrome |
| Disorders | Charcot-Marie-Tooth type 2 |
| | Primary Mitochondrial Diseases (Disorders) including |
| | NARP, MELAS, Chronic Progressive External |
| | Ophthalmoplegia, Leigh’s disease, Leber’s Hereditary |
| | Optic Neuropathy, MERRF, Barth Syndrome, Luft |
| | Disease, Kearns Sayre Syndrome, Autosomal dominant |
| | optic atrophy |
| | Friedreich's ataxia |
| | Werner syndrome |
| General | Tissue repair following physical trauma, hemorrhagic |
| | shock, tissue grafting, organ transplant including heart, |
| | lung, liver, and kidney |
| | Stem cell therapies, including hematopoietic stem cell |
| | transfer, allogenic mesenchymal stem therapy for acute |
| | graft-vs-host disease, limbal stem cell deficiency due to |
| | genetic or acquired conditions that compromise normal |
| | turnover of the corneal epithelium |

In some embodiments, the disease or condition mediated by CD38 activity is cancer and chemotherapy-induced tissue damage, a cardiovascular disease, a renal disease, chronic inflammatory and fibrotic disease, a vascular disease, metabolic dysfunction, a muscular disease, a neurological disease or injury, or a DNA damage disorder or primary mitochondrial disorder. Provided in some embodiments are methods of treating a disease or condition mediated by CD38 activity in a subject in need thereof, comprising administering to the individual or subject in need thereof a compound of formula (I), (I-A), (I-A1), (I-A2), (I-B), (I-C), (I-D), (I-E), (I-F), (I-G), (I-H), or (I-J), or a compound of Table 1, or a pharmaceutically acceptable salt thereof. In some embodiments, the disease or condition is cancer or chemotherapy induced tissue damage, a cardiovascular disease, a renal disease, a chronic inflammatory or fibrotic disease, a vascular disease, metabolic dysfunction, a muscular disease, a neurological disease or injury, a DNA damage disorder or Primary Mitochondrial Disorder, including any of the diseases listed in Table 2.

### Dosages

The compounds and compositions disclosed and/or described herein are administered at a therapeutically effective dosage, e.g., a dosage sufficient to provide treatment for the disease state. While human dosage levels have yet to be optimized for the chemical entities described herein, generally, a daily dose ranges from about 0.01 to 100 mg/kg of body weight; in some embodiments, from about 0.05 to 10.0 mg/kg of body weight, and in some embodiments, from about 0.10 to 1.4 mg/kg of body weight. Thus, for administration to a 70 kg person, in some embodiments, the dosage range would be about from 0.7 to 7000 mg per day; in some embodiments, about from 3.5 to 700.0 mg per day, and in some embodiments, about from 7 to 100.0 mg per day. The amount of the chemical entity administered will be dependent, for example, on the subject and disease state being treated, the severity of the affliction, the manner and schedule of administration and the judgment of the prescribing physician. For example, an exemplary dosage range for oral administration is from about 5 mg to about 500 mg per day, and an exemplary intravenous administration dosage is from about 5 mg to about 500 mg per day, each depending upon the compound pharmacokinetics.

A daily dose is the total amount administered in a day. A daily dose may be, but is not limited to be, administered each day, every other day, each week, every 2 weeks, every month, or at a varied interval. In some embodiments, the daily dose is administered for a period ranging from a single day to the life of the subject. In some embodiments, the daily dose is administered once a day. In some embodiments, the daily dose is administered in multiple divided doses, such as in 2, 3, or 4 divided doses. In some embodiments, the daily dose is administered in 2 divided doses.

Administration of the compounds and compositions disclosed and/or described herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the compound or composition is administered orally or intravenously. In some embodiments, the compound or composition disclosed and/or described herein is administered orally.

Pharmaceutically acceptable compositions include solid, semi-solid, liquid and aerosol dosage forms, such as tablet, capsule, powder, liquid, suspension, suppository, and aerosol forms. The compounds disclosed and/or described herein can also be administered in sustained or controlled release dosage forms (e.g., controlled/sustained release pill, depot injection, osmotic pump, or transdermal (including electrotransport) patch forms) for prolonged timed, and/or pulsed administration at a predetermined rate. In some embodiments, the compositions are provided in unit dosage forms suitable for single administration of a precise dose.

The compounds disclosed and/or described herein can be administered either alone or in combination with one or more conventional pharmaceutical carriers or excipients (e.g., mannitol, lactose, starch, magnesium stearate, sodium saccharine, talcum, cellulose, sodium crosscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate). Generally, depending on the intended mode of administration, the pharmaceutical composition will contain about 0.005% to 95%, or about 0.5% to 50%, by weight of a compound disclosed and/or described herein. Actual methods of preparing such dosage forms are known, or will be apparent, to those skilled in this art; for example, see Remington's Pharmaceutical Sciences, Mack Publishing Company, Easton, Pa.

In some embodiments, the compositions will take the form of a pill or tablet and thus the composition may contain, along with a compounds disclosed and/or described herein, one or more of a diluent (e.g., lactose, sucrose, dicalcium phosphate), a lubricant (e.g., magnesium stearate), and/or a binder (e.g., starch, gum acacia, polyvinylpyrrolidine, gelatin, cellulose, cellulose derivatives). Other solid dosage forms include a powder, marume, solution or suspension (e.g., in propylene carbonate, vegetable oils or triglycerides) encapsulated in a gelatin capsule.

Liquid pharmaceutically administrable compositions can, for example, be prepared by dissolving, dispersing or suspending etc. a compound disclosed and/or described herein and optional pharmaceutical additives in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of the compound contained in such parenteral compositions depends, for example, on the physical nature of the compound, the activity of the compound and the needs of the subject. However, percentages of active ingredient of 0.01% to 10% in solution are employable, and may be higher if the composition is a solid which will be subsequently diluted to another concentration. In some embodiments, the composition will comprise from about 0.2 to 2% of a compound disclosed and/or described herein in solution.

Pharmaceutical compositions of the compounds disclosed and/or described herein may also be administered to the respiratory tract as an aerosol or solution for a nebulizer, or as a microfine powder for insufflation, alone or in combination with an inert carrier such as lactose. In such a case, the particles of the pharmaceutical composition may have diameters of less than 50 microns, or in some embodiments, less than 10 microns.

In addition, pharmaceutical compositions can include a compound disclosed and/or described herein and one or more additional medicinal agents, pharmaceutical agents, adjuvants, and the like. Suitable medicinal and pharmaceutical agents include those described herein.

### Kits

Also provided are articles of manufacture and kits containing any of the compounds or pharmaceutical compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing a compound or composition described herein and instructions for use. The kits may contain instructions for use in the treatment of a heart disease in an individual or subject in need thereof. A kit may additionally contain any materials or equipment that may be used in the administration of the compound or composition, such as vials, syringes, or IV bags. A kit may also contain sterile packaging.

### Combinations

The compounds and compositions described and/or disclosed herein may be administered alone or in combination with other therapies and/or therapeutic agents useful in the treatment of the aforementioned disorders, diseases, or conditions.

### General Synthetic Methods

Compounds of formula (I), or any variation or embodiment thereof, or a salt of any of the foregoing, will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or side chain groups) from reaction conditions, and that such groups are removed under standard conditions when appropriate. Unless otherwise specified, the variables are as defined above in reference to formula (I).

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g. a racemate, and an appropriate chiral compound. The diastereomers may then be separated by any convenient means, for example, by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

The following abbreviations may be used throughout the Schemes and Examples: TEA (triethylamine), DCM (dichloromethane), (Boc)₂O (di-tert-butyl dicarbonate), EA (Ethyl acetate), PE (Petroleum ether), DMF (N,N-dimethylformamide), DIEA (N-ethyl-N-isopropylpropan-2-amine), HATU (1-[Bis(dimethylamino)methylene]-1H-1,2,3-triazolo[4,5-b]pyridinium 3-oxid hexafluorophosphate), HOAt (1-Hydroxy-7-azabenzotriazole), HOBt (Hydroxybenzotriazole), EDC or EDCI (1-Ethyl-3-(3-dimethylaminopropyl)carbodiimide), MeOH (methanol), EtOH (ethanol), iPrOH (propan-2-ol), ACN (acetonitrile), TFA (trifluoroacetic acid), DPPA (Diphenylphosphoryl azide), DBU (1,8-Diazabicyclo(5.4.0)undec-7-ene), THF (tetrahydrofuran), PPh₃ (triphenylphosphane), SM (starting material), Hex (hexane), NCS (N-chlorosuccinimide), r.t. (room temperature), DCE (dichloroethane), FA (formic acid), CHCl₃ (Chloroform), BnBr (benzyl bromide), HCl (hydrogen chloride), equiv (equivalent), and DSC (bis(2,5-dioxopyrrolidin-1-yl) carbonate), HBTU (O-(benzotriazol-1-yl)-N,N,N′,N′-tetramethyluronium hexafluorophosphate), NMP (N-methyl-2-pyrrolidone), dppf (1,1′-bis(diphenylphosphino)ferrocene), T₃P (Propylphosphonic anhydride), LHMDS (Lithium bis(trimethylsilyl)amide), Alk (alkyl), Pybrop (Bromo-tris-pyrrolidino-phosphonium hexafluorophosphate), h (hour), min (minute).

Note that, in each of the following Schemes, the various moieties are as defined for a compound of formula (I), or any variation or embodiment thereof, or a stereoisomer or tautomer thereof, or a pharmaceutically acceptable salt of any of the foregoing.

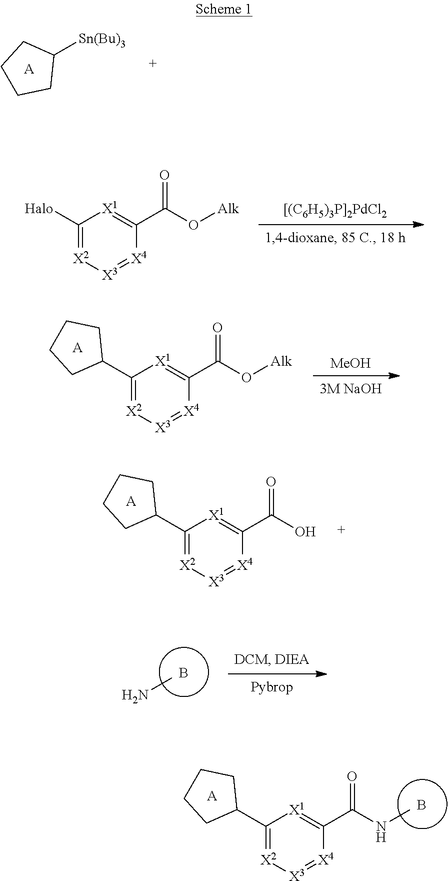

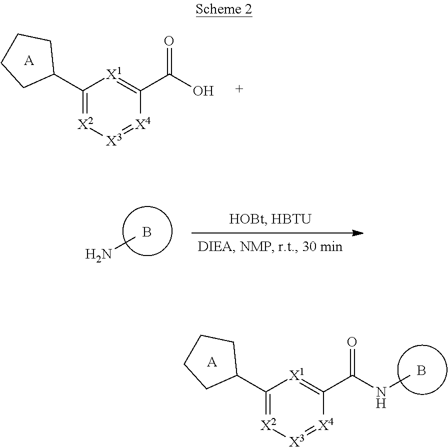

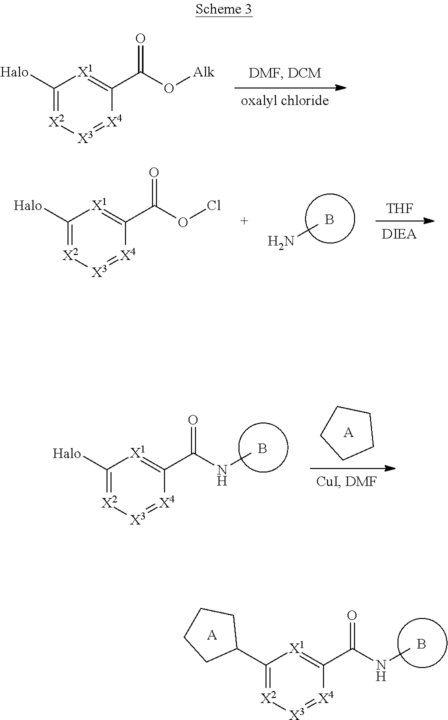

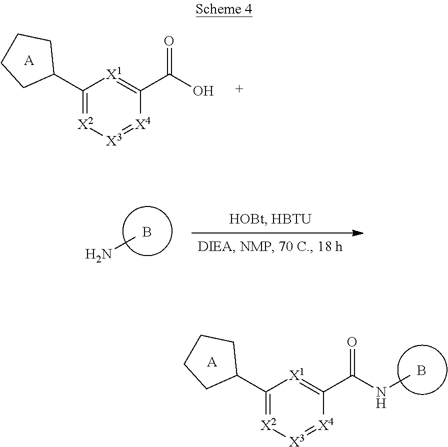

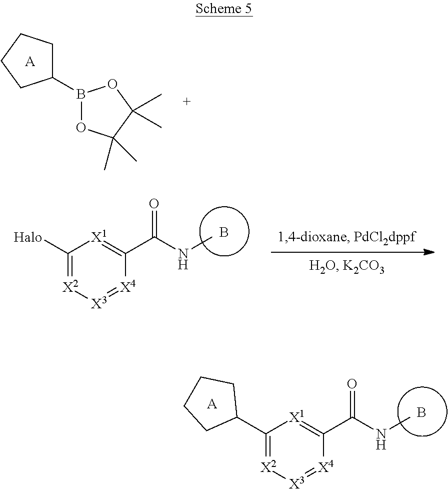

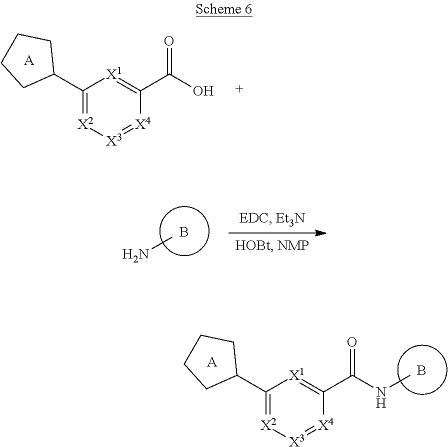

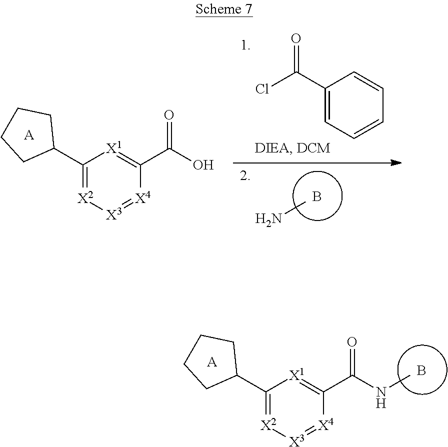

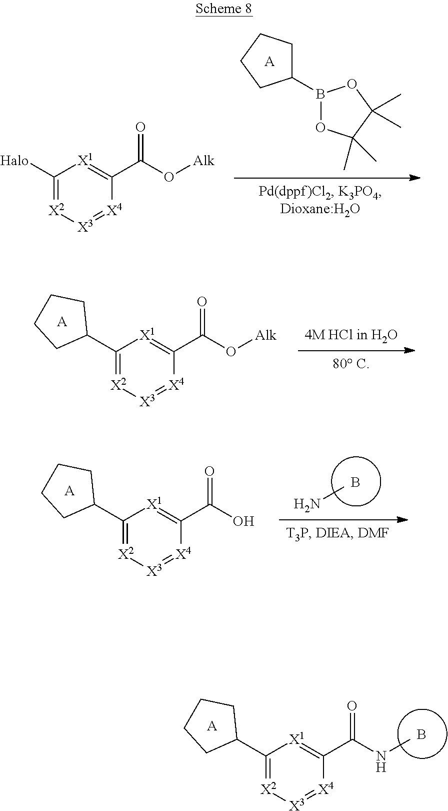

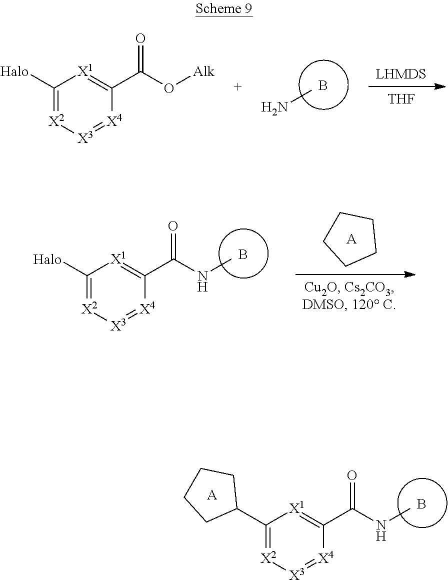

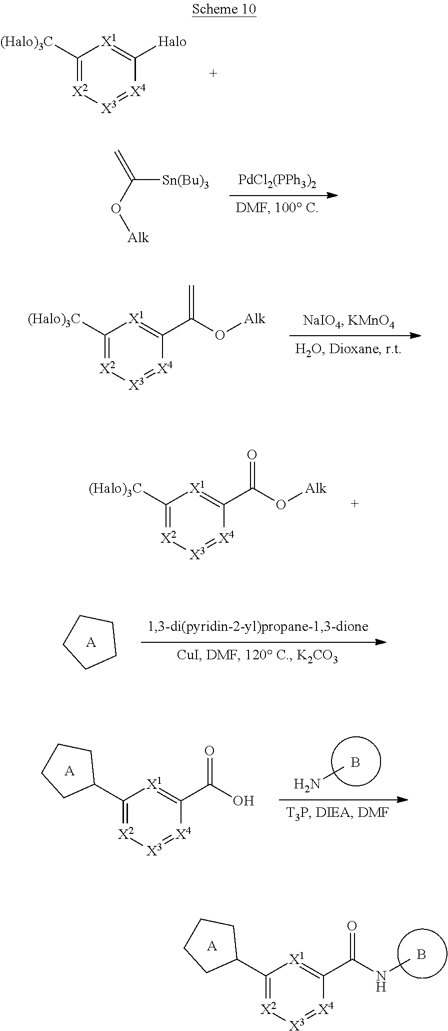

Particular non-limiting examples are provided in the Example section below. Note that, in the Examples, the compound numbers correspond to those in Table 1.

### ENUMERATED EMBODIMENTS

The following enumerated embodiments are representative of some aspects of the present disclosure.

Embodiment 1. A compound of formula (I):

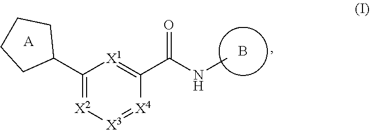

or a stereoisomer or tautomer thereof, or a pharmaceutically acceptable salt of any of the foregoing, wherein:

### X¹ is N or CH,

X² is N or C(R^{x}), wherein R^{x} is H, halo, or C₁₋₆alkyl,
X³ is N or C(R^{y}), wherein R^{y} is H, —OH, C₁₋₆alkoxy, C₃₋₁₀cycloalkyl, 3-10 membered heterocyclyl, or C₁₋₆alkyl, wherein the C₁₋₆alkyl of R^{y} is optionally substituted with one or more halo or —OH, and
X⁴ is N or C(R^{z}), wherein R^{z} is H, halo, or C₁₋₆alkyl, provided that at most two of X¹, X², X³, and X⁴ are N;

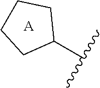

is:
(i)

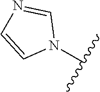 [SMILES: *n1ccnc1]

that is optionally substituted with one or more —C(O)—NH₂, or
(ii)

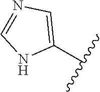 [SMILES: *c1cnc[nH]1]

that is optionally substituted with one or more C₁₋₆ alkyl, or
(iii)

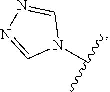 [SMILES: *n1cnnc1]

or
(iv)

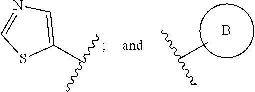

is:
saturated C₄₋₈cycloalkyl, wherein the C₄₋₈cycloalkyl is optionally substituted with one or more R^{a}, wherein each R^{a} is independently —OH, halo, C₁₋₆alkyl, C₁₋₆haloalkyl, C₁₋₆alkoxy, or —C(O)—C₁₋₆ alkoxy, wherein the C₁₋₆alkoxy of R^{a} is optionally substituted with one or more C₁₋₆alkoxy and the C₁₋₆ alkyl of R^{a} is optionally substituted with one or more —OH or C₁₋₆alkoxy, or
(ii) saturated 4-8 membered heterocyclyl, wherein the 4-8 membered heterocyclyl is optionally substituted with one or more R^{b}, wherein each R^{b} is independently oxo, —C(O)—C₁₋₆alkyl, —C(O)—C₁₋₆ alkoxy, or phenyl, wherein the phenyl of R^{b} is optionally substituted with one or more C₁₋₆haloalkyl, or
(iii) phenyl, wherein the phenyl is optionally substituted with one or more halo, or
(iv) pyridinyl, wherein the pyridinyl is optionally substituted with one or more halo, C₁₋₆ alkyl, C₁₋₆haloalkyl, or C₁₋₆ alkoxy.

Embodiment 2. The compound of embodiment 1, or a stereoisomer or tautomer thereof, or a pharmaceutically acceptable salt of any of the foregoing, wherein exactly one of X¹, X², X³, and X⁴ is N.

Embodiment 3. The compound of embodiment 1 or embodiment 2, or a stereoisomer or tautomer thereof, or a pharmaceutically acceptable salt of any of the foregoing, wherein the compound is of formula

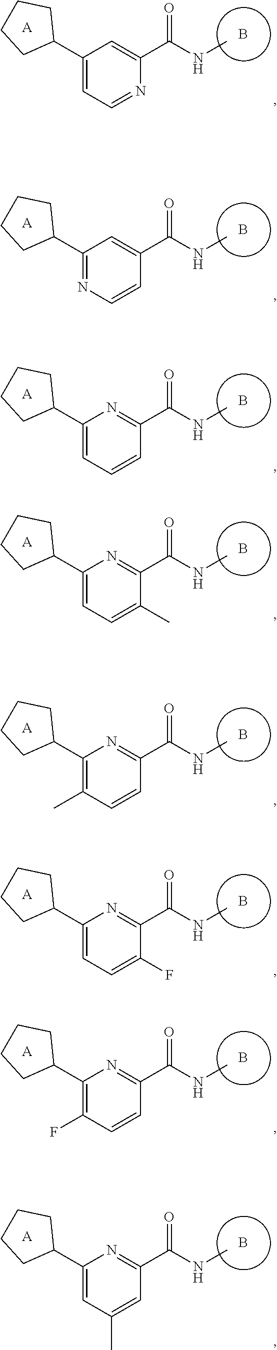

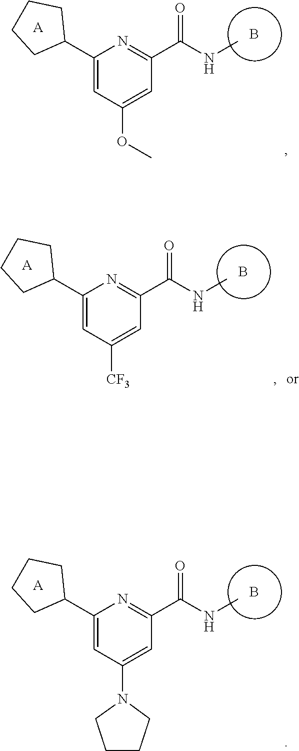

Embodiment 4. The compound of embodiment 1, or a stereoisomer or tautomer thereof, or a pharmaceutically acceptable salt of any of the foregoing, wherein exactly two of X¹, X², X³, and X⁴ are N.

Embodiment 5. The compound of embodiment 1 or embodiment 4, or a stereoisomer or tautomer thereof, or a pharmaceutically acceptable salt of any of the foregoing, wherein the compound is of formula

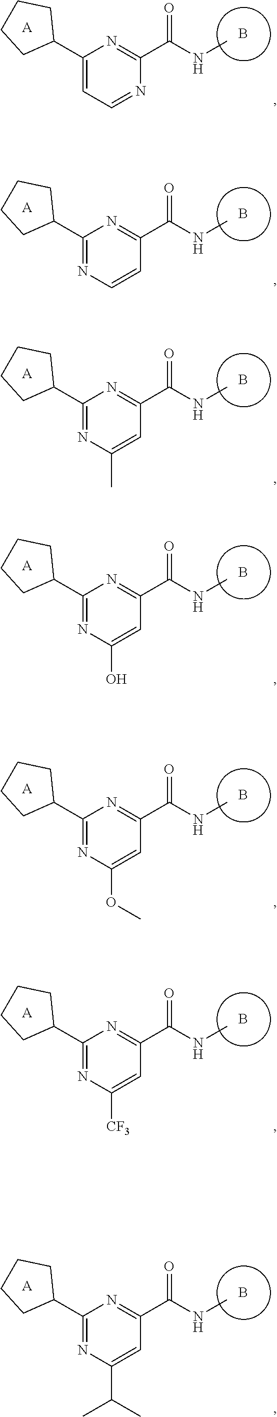

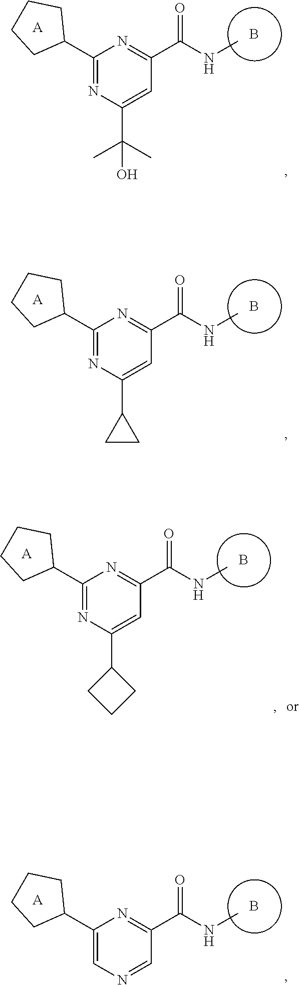

Embodiment 6. The compound of embodiment 1, or a stereoisomer or tautomer thereof, or a pharmaceutically acceptable salt of any of the foregoing, wherein the compound is of formula

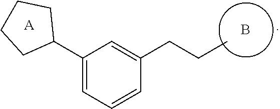

Embodiment 7. The compound of any one of embodiments 1-6, or a stereoisomer or tautomer thereof, or a pharmaceutically acceptable salt of any of the foregoing, wherein

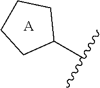

is

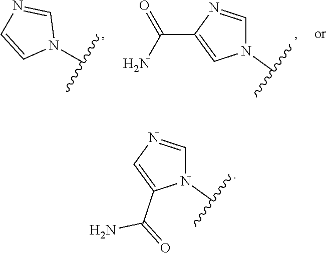 [SMILES: *n1ccnc1 ; *n1cnc(C(N)=O)c1 ; *n1cncc1C(N)=O]

Embodiment 8. The compound of any one of embodiments 1-7, or a stereoisomer or tautomer thereof, or a pharmaceutically acceptable salt of any of the foregoing, wherein

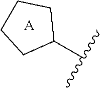

is

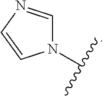 [SMILES: *n1ccnc1]

Embodiment 9. The compound of any one of embodiments 1-6, or a stereoisomer or tautomer thereof, or a pharmaceutically acceptable salt of any of the foregoing, wherein

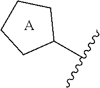

is

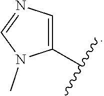 [SMILES: *c1cncn1C]

Embodiment 10. The compound of any one of embodiments 1-6, or a stereoisomer or tautomer thereof, or a pharmaceutically acceptable salt of any of the foregoing, wherein

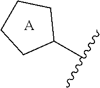

is

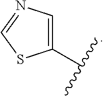 [SMILES: *c1cncs1]

Embodiment 11. The compound of any one of embodiments 1-6, or a stereoisomer or tautomer thereof, or a pharmaceutically acceptable salt of any of the foregoing, wherein

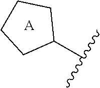

is

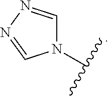 [SMILES: *n1cnnc1]

Embodiment 12. The compound of any one of embodiments 1-11, or a stereoisomer or tautomer thereof, or a pharmaceutically acceptable salt of any of the foregoing, wherein

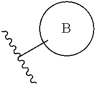

is saturated C₄₋₈ cycloalkyl, wherein the C₄₋₈ cycloalkyl is optionally substituted with one or more R^{a}, wherein each R^{a} is independently —OH, halo, C₁₋₆alkyl, C₁₋₆haloalkyl, C₁₋₆alkoxy, or —C(O)—C₁₋₆ alkoxy, wherein the C₁₋₆ alkoxy of R^{a} is optionally substituted with one or more C₁₋₆ alkoxy and the C₁₋₆ alkyl of R^{a} is optionally substituted with one or more —OH or C₁₋₆alkoxy.

Embodiment 13. The compound of any one of embodiments 1-12, or a stereoisomer or tautomer thereof, or a pharmaceutically acceptable salt of any of the foregoing, wherein

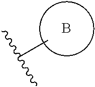

is

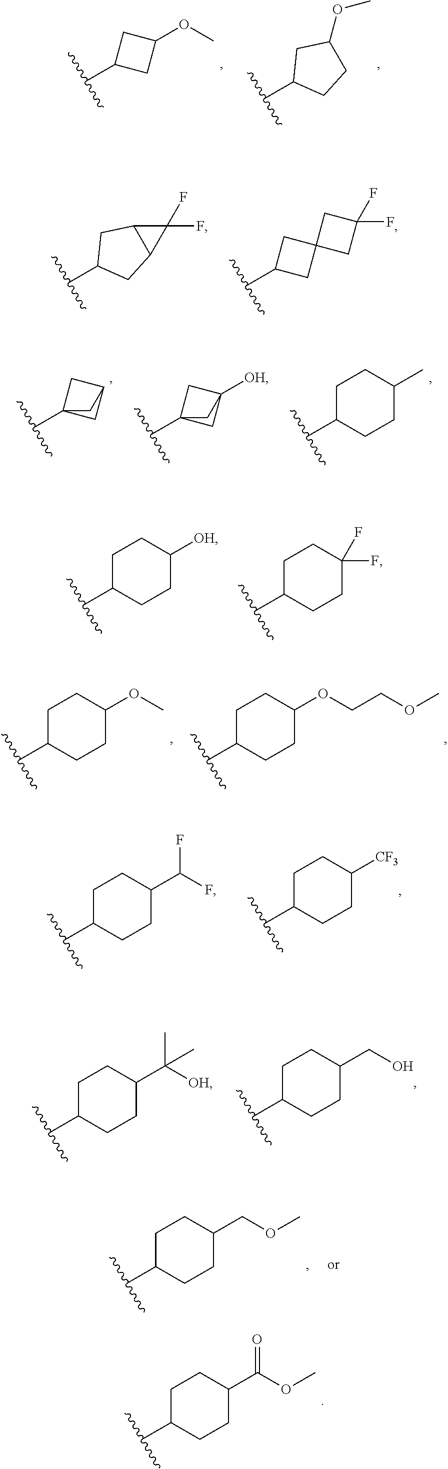

Embodiment 14. The compound of any one of embodiments 1-11, or a stereoisomer or tautomer thereof, or a pharmaceutically acceptable salt of any of the foregoing, wherein

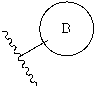

is saturated 4-8 membered heterocyclyl, wherein the 4-8 membered heterocyclyl is optionally substituted with one or more R^{b}, wherein each R^{b} is independently oxo, —C(O)—C₁₋₆alkyl, —C(O)—C₁₋₆ alkoxy, or phenyl, wherein the phenyl of R^{b} is optionally substituted with one or more C₁₋₆ haloalkyl.

Embodiment 15. The compound of any one of embodiments 1-11 and 14, or a stereoisomer or tautomer thereof, or a pharmaceutically acceptable salt of any of the foregoing, wherein

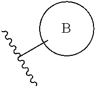

is

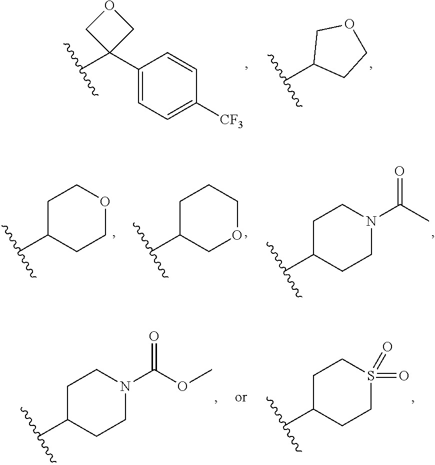

Embodiment 16. The compound of any one of embodiments 1-11, or a stereoisomer or tautomer thereof, or a pharmaceutically acceptable salt of any of the foregoing, wherein

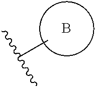

is phenyl, wherein the phenyl is optionally substituted with one or more halo.

Embodiment 17. The compound of any one of embodiments 1-11 and 16, or a stereoisomer or tautomer thereof, or a pharmaceutically acceptable salt of any of the foregoing, wherein

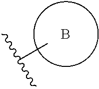

is

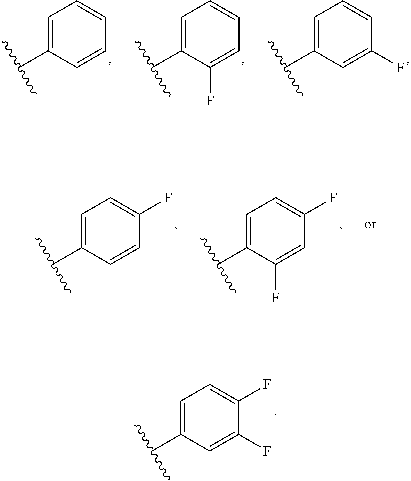 [SMILES: *c1ccccc1 ; *c1ccccc1F ; *c1cccc(F)c1 ; *c1ccc(F)cc1 ; *c1ccc(F)cc1F ; *c1ccc(F)c(F)c1]

Embodiment 18. The compound of any one of embodiments 1-11, or a stereoisomer or tautomer thereof, or a pharmaceutically acceptable salt of any of the foregoing, wherein

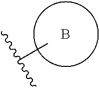

is pyridinyl, wherein the pyridinyl is optionally substituted with one or more halo, C₁₋₆alkyl, C₁₋₆haloalkyl, or C₁₋₆ alkoxy.

Embodiment 19. The compound of any one of embodiments 1-11 and 18, or a pharmaceutically acceptable salt of any of the foregoing, wherein

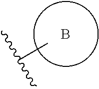

is

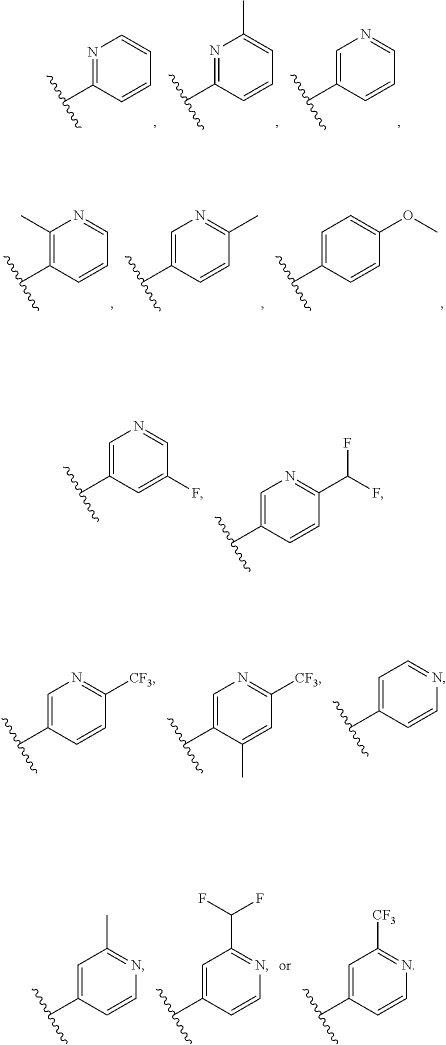

Embodiment 20. A compound selected from the group consisting of the compounds of Table 1, or a stereoisomer or tautomer thereof, or a pharmaceutically acceptable salt of any of the foregoing.

Embodiment 21. A pharmaceutical composition, comprising: (i) an effective amount of a compound of any one of embodiments 1-20, or a stereoisomer or tautomer thereof, or a pharmaceutically acceptable salt of any of the foregoing; and (ii) one or more pharmaceutically acceptable excipients.

Embodiment 22. A method of treating a disease, disorder, or condition mediated by CD38 activity in a subject in need thereof, comprising administering to the subject an effective amount of a compound of any one of embodiments 1-20, or a stereoisomer or tautomer thereof, or a pharmaceutically acceptable salt of any of the foregoing, or a pharmaceutical composition of embodiment 21.

Embodiment 23. The method of embodiment 22, wherein the disease, disorder, or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, and a muscle disease or muscle wasting disorder.

Embodiment 24. The method of embodiment 22, wherein the disease, disorder, or condition is selected from the group consisting of obesity, atherosclerosis, insulin resistance, type 2 diabetes, cardiovascular disease, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury.

### EXAMPLES

The following examples are offered to illustrate but not to limit the compositions, uses, and methods provided herein. The compounds are prepared using the general methods described above.

### Example A

### Synthesis of Compound 11

### Preparation of N-(pyridin-3-yl)-6-(thiazol-5-yl)picolinamide (Compound 11)

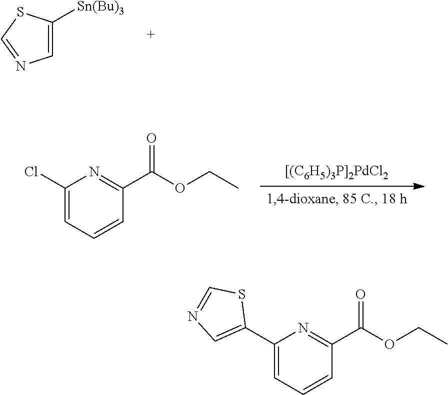

Step 1: Preparation of Ethyl 6-(thiazol-5-yl)picolinate. 5-(tributylstannyl)thiazole (800 mg, 2.14 mmol) was combined with ethyl 6-chloropicolinate (397 mg, 2.14 mmol) and anhydrous 1,4-dioxane (15 mL) was added followed by trans-dichlorobis(triphenylphosphine)palladium(II) (150 mg, 0.21, mmol). The resulting mixture was heated in an oil bath at 85° C. for 18 h. The solvent was removed under reduced pressure and the product was purified with silica gel using 40% ethyl acetate/hexanes, providing ethyl 6-(thiazol-5-yl)picolinate (139 mg, 0.59 mmol, 28%) as an off-white solid which was used in the subsequent step without additional purification. LRMS (APCI) m/z 234.9 (M+H).

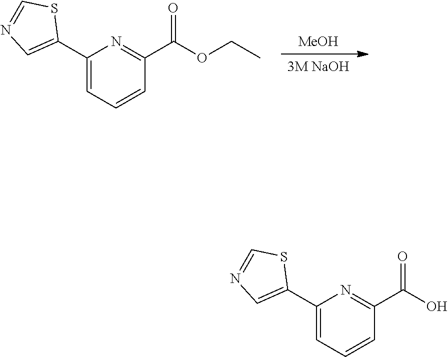 [SMILES: "MeOH" ; CCOC(=O)c1cccc(-c2cncs2)n1 ; "3M NaOH" ; O=C(O)c1cccc(-c2cncs2)n1]

Step 2: Preparation of 6-(Thiazol-5-yl)picolinic acid. Ethyl 6-(thiazol yl)picolinate (139 mg, 0.59 mmol) was dissolved in MeOH (3 mL) and 3 M aq. NaOH (2 mL, 6.0 mmol) was added. The mixture was stirred at 80° C. for 15 min, the MeOH was evaporated under reduced pressure and the pH of the remaining aqueous phase was adjusted to 4 using concentrated aq. HCl. The resulting suspension was filtered providing 6-(thiazol-5-yl)picolinic acid (44 mg, 0.21 mmol, 36%) as a white solid which was used in the subsequent step without additional purification. LRMS (APCI) m/z 207.0 (M+H).

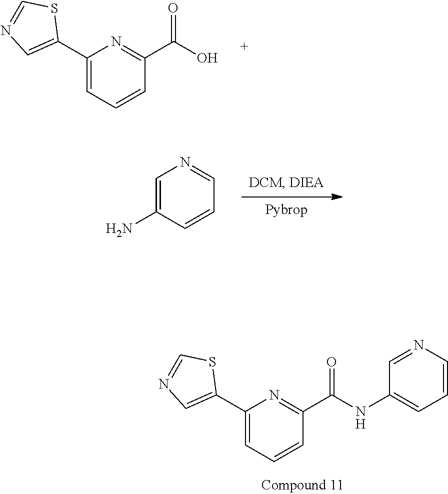 [SMILES: O=C(O)c1cccc(-c2cncs2)n1 ; Nc1cccnc1 ; "DCM, DIEA" ; "Pybrop" ; O=C(Nc1cccnc1)c1cccc(-c2cncs2)n1]

Step 3: Preparation of N-(Pyridin-3-yl)-6-(thiazol-5-yl)picolinamide. 6-(Thiazol-5-yl)picolinic acid (17 mg, 0.082 mmol) was combined with pyridin-3-amine (12 mg, 0.124 mmol). DCM (2 mL) was added, followed by bromotripyrrolidinophosphonium hexafluorophosphate (56 mg, 0.124 mmol) and DIEA (43 mL, 0.247 mmol). The reaction was stirred at r.t. for 15 min, the solvent was evaporated under reduced pressure and the product was purified using reverse phase HPLC with a 40 minute gradient from 5-100% ACN/water with 0.1% formic acid in both phases (Phenomenex Gemini 5 micron C18 Axia pack 150×21.2 mm column) providing N-(pyridin-3-yl)-6-(thiazol-5-yl)picolinamide (15 mg, 0.053 mmol, 64%) as a white solid. LRMS (APCI) m/z 283.0 (M+H). ¹H NMR (400 MHz, Methanol-d₄) δ 9.14 (s, 1H), 9.09 (s, 1H), 8.70 (s, 1H), 8.49-8.35 (m, 2H), 8.21-8.05 (m, 3H), 7.57 (dd, J=7.9, 5.1 Hz, 1H).

### Example B

### Synthesis of Compound 12

### Preparation of 6-(1H-imidazol-1-yl)-N-(tetrahydro-2H-pyran-4-yl)picolinamide (Compound 12)

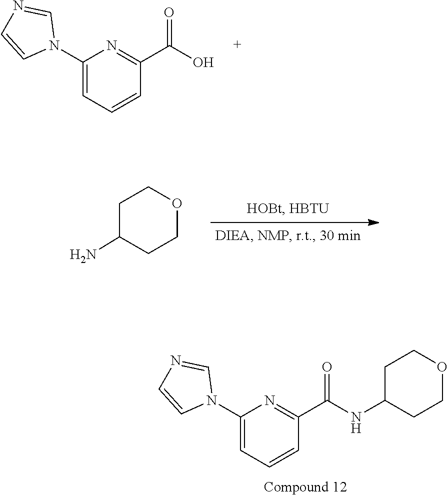 [SMILES: O=C(O)c1cccc(-n2ccnc2)n1 ; NC1CCOCC1 ; O=C(NC1CCOCC1)c1cccc(-n2ccnc2)n1]

Preparation of 6-(1H-imidazol-1-yl)-N-(tetrahydro-2H-pyran-4-yl)picolinamide (Compound 12). 6-(1H-imidazol-1-yl)picolinic acid (49 mg, 0.259 mmol) was combined with tetrahydro-2H-pyran-4-amine (31 mg, 0.311 mmol), HBTU (147 mg, 0.389 mmol), HOBt (52 mg, 0.389 mmol) and N-methylpyrrolidone (2 mL). DIEA (135 mL, 0.777 mmol) was added and the mixture was stirred at r.t. for 30 min. The product was purified using reverse phase HPLC with a 40 minute gradient from 5-100% ACN/water (Phenomenex Gemini 5 micron C18 Axia pack 150×21.2 mm column) providing 6-(1H-imidazol-1-yl)-N-(tetrahydro-2H-pyran yl)picolinamide (32 mg, 0.118 mmol, 45%) as a white solid. LRMS (APCI) m/z 273.1 (M+H).

¹H NMR (400 MHz, Methanol-d₄) δ 8.84 (s, 1H), 8.19-8.04 (m, 3H), 7.89 (d, J=8.1 Hz, 1H), 7.19 (s, 1H), 4.25-4.11 (m, 1H), 4.01 (d, J=11.0 Hz, 2H), 3.55 (td, J=11.6, 2.1 Hz, 2H), 1.97-1.71 (m, 4H).

### Example C

### Synthesis of Compound 13

### Preparation of 6-(1H-imidazol-1-yl)-N-(6-(trifluoromethyl)pyridin-3-yl)picolinamide (Compound 13)

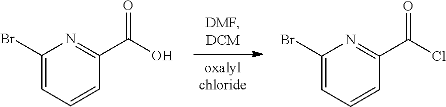 [SMILES: O=C(O)c1cccc(Br)n1 ; O=C(Cl)c1cccc(Br)n1]

Step 1: Preparation of 6-Bromopicolinoyl chloride. 6-Bromopicolinic acid (1.46 g, 7.22 mmol) was suspended in DCM (10 mL) and oxalyl chloride (3.97 mL of 2.0 M in DCM, 7.94 mmol) was added followed by DMF (53 mg, 0.72 mmol). The mixture was stirred at r.t. for 30 min during which time a homogeneous solution was observed. The solvents were concentrated in vacuo providing 6-bromopicolinoyl chloride (1.59 g, 7.22 mmol, 100%) as a tan solid which was dried under high vacuum and used in the next step without additional purification.

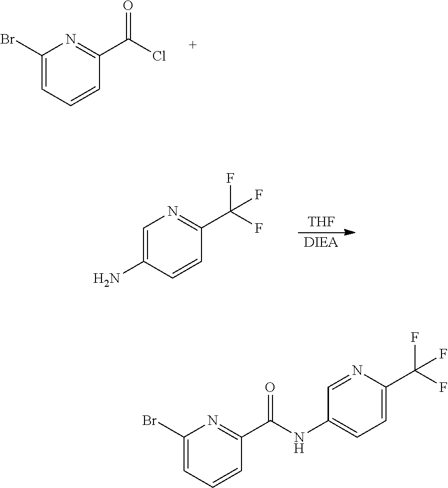 [SMILES: O=C(Cl)c1cccc(Br)n1 ; Nc1ccc(C(F)(F)F)nc1 ; "DIEA" ; O=C(Nc1ccc(C(F)(F)F)nc1)c1cccc(Br)n1 ; "THF"]

Step 2: Preparation of 6-Bromo-N-(6-(trifluoromethyl)pyridin-3-yl)picolinamide. 6-bromopicolinoyl chloride (1.43 g, 6.49 mmol) was dissolved in THF (10 mL) and 6-(trifluoromethyl)pyridin-3-amine (1.05 g, 6.49 mmol) was added followed by DIEA (3.39 mL, 19.5 mmol). The resulting mixture was stirred at r.t. for 15 min., diluted with ethyl acetate (50 mL), washed with water (50 mL) and brine, dried over sodium sulfate and concentrated under reduced pressure. The product was purified with silica gel using 30% ethyl acetate/hexanes providing 6-bromo-N-(6-(trifluoromethyl)pyridin-3-yl)picolinamide (1.91 g, 5.53 mmol, 85%) as an off-white solid. LRMS (APCI) m/z 345.9 (M+H).

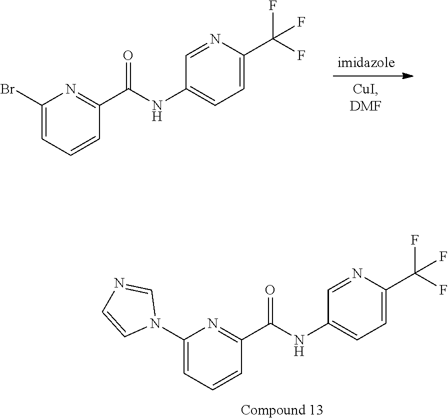 [SMILES: O=C(Nc1ccc(C(F)(F)F)nc1)c1cccc(Br)n1 ; O=C(Nc1ccc(C(F)(F)F)nc1)c1cccc(-n2ccnc2)n1]

Step 3: Preparation of 6-(1H-imidazol-1-yl)-N-(6-(trifluoromethyl)pyridin-3-yl)picolinamide. 6-bromo-N-(6-(trifluoromethyl)pyridin-3-yl)picolinamide (918 mg, 2.65 mmol) was combined with imidazole (271 mg, 3.98 mmol), CuI (253 mg, 1.33 mmol) and K-₂CO₃ (1.11 g, 7.96 mmol). DMF (6 mL) was added and the mixture was heated in a microwave at 150° C. for 30 min, diluted with ethyl acetate (20 mL), water (20 mL) and filtered through celite. Additional ethyl acetate was added (60 mL) and the layers were separated. The organic phase was washed with brine, dried over sodium sulfate and concentrated under reduced pressure. The product was purified using reverse phase HPLC with a 40 minute gradient from 5-100% ACN/water (Phenomenex Gemini 5 micron C18 Axia pack 150×21.2 mm column) followed by trituration with diethyl ether and filtration to give 6-(1H-imidazol-1-yl)-N-(6-(trifluoromethyl)pyridin-3-yl)picolinamide (302 mg, 0.91 mmol, 34%) as a white solid. LRMS (APCI) m/z 345.9 (M+H). ¹H NMR (400 MHz, DMSO-d₆) δ 10.92 (s, 1H), 9.25 (s, 1H), 9.07 (s, 1H), 8.60 (d, J=8.6 Hz, 1H), 8.40 (s, 1H), 8.29 (t, J=7.8 Hz, 1H), 8.13 (t, J=6.8 Hz, 2H), 7.99 (d, J=8.6 Hz, 1H), 7.26 (s, 1H).

Compounds 14-16 were prepared in a similar manner as Compound 12, using the amines provided in the table below in place of tetrahydro-2H-pyran-4-amine.

| Compound | |
|---|---|
| Number | Amine |
| 14 | (1r,4r)-4-(2-methoxyethoxy)cyclohexan-1-amine |
| 15 | 3-aminobicyclo[1.1.1]pentan-1-ol |
| 16 | bicyclo[1.1.1] pentan-1-amine |

### Example D

### Synthesis of Compound 17

### Preparation of N-(2-fluorophenyl)-6-(1H-imidazol-1-yl)picolinamide (Compound 17)

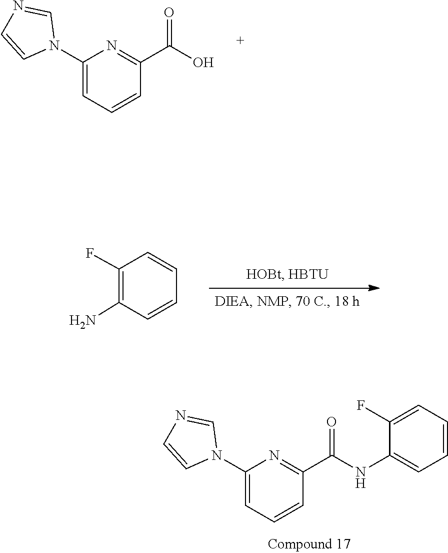 [SMILES: O=C(O)c1cccc(-n2ccnc2)n1 ; Nc1ccccc1F ; O=C(Nc1ccccc1F)c1cccc(-n2ccnc2)n1]

Preparation of N-(2-fluorophenyl)-6-(1H-imidazol-1-yl)picolinamide (Compound 17). 6-(1H-imidazol-1-yl)picolinic acid (56 mg, 0.296 mmol) was combined with 2-fluoroaniline (39 mg, 0.355 mmol), HBTU (168 mg, 0.444 mmol), HOBt (60 mg, 0.444 mmol) and N-methylpyrrolidone (2 mL). DIEA (155 mL, 0.888 mmol) was added and the mixture was stirred at 70° C. for 18 h. The reaction was cooled to r.t. and the product was purified using reverse phase HPLC with a 40 minute gradient from 5-100% ACN/water (Phenomenex Gemini 5 micron C18 Axia pack 150×21.2 mm column) providing N-(2-fluorophenyl)-6-(1H-imidazol-1-yl)picolinamide (18 mg, 0.064 mmol, 22%) as a white solid. LRMS (APCI) m/z 283.1 (M+H).

¹H NMR (400 MHz, Methanol-d4) δ 8.80 (s, 1H), 8.28-8.04 (m, 4H), 7.98 (d, J=7.5 Hz, 1H), 7.32-7.18 (m, 4H).

Compounds 18-35 and 38-43 were prepared using the methods provided in the table below.

| Compound | |
|---|---|
| Number | Method of Preparation |
| 18 | Prepared in same fashion as Compound 17 |
| 19 | Prepared in same fashion as Compound 17 |
| 20 | Prepared in same fashion as Compound 17 |
| 21 | Prepared in same fashion as Compound 17 |
| 22 | Beginning with methyl 6-bromo-5-methylpicolinate, |
| | ester hydrolysis as |
| | Compound 11 followed by amide bond |
| | formation as Compound 17 and CuI |
| | coupling as Compound 13 |
| 23 | Prepared in same fashion as Compound 12 |
| 24 | Prepared in same fashion as Compound 17 |
| 25 | Prepared in same fashion as Compound 17 |
| 26 | Beginning with 6-bromo-3-methylpicolinic |
| | acid, amide bond formation as |
| | Compound 17 followed by Cui coupling |
| | as Compound 13 |
| 27 | Prepared in same fashion as Compound 17 |
| 28 | Prepared in same fashion as Compound 13 |
| 29 | Prepared in same fashion as Compound 17 |
| 30 | Prepared in same fashion as Compound 12 |
| 31 | Prepared in same fashion as Compound 12 |
| 32 | Beginning with 6-bromo-4-methylpicolinic |
| | acid, amide bond formation as Compound 17 |
| | followed by CuI coupling as Compound 13 |
| 33 | Prepared in same fashion as Compound 17 |
| 34 | Prepared in same fashion as Compound 17 |
| 35 | Beginning with 6-bromo-4-methylpicolinic |
| | acid, amide bond formation as Compound 17 |
| | followed by Stille coupling as Compound 11 |
| 38 | Amide bond formation as Compound 17 |
| | followed by Cui coupling as Compound 13 |
| 39 | Amide bond formation as Compound 17 |
| | followed by Cui coupling as Compound 13 |
| 40 | Beginning with |
| | 6-bromo-4-(trifluoromethyl)picolinic acid, |
| | amide bond formation as Compound 17 |
| | followed by Cui coupling as Compound 13 |
| 41 | Beginning with |
| | 6-bromo-4-(trifluoromethyl)picolinic acid, |
| | amide bond formation as Compound 17 |
| | followed by Stille coupling as Compound 11 |
| 42 | Beginning with 6-bromo-3-fluoropicolinic |
| | acid, prepared in same fashion as |
| | Compound 13 |
| 43 | Beginning with 6-bromo-3-fluoropicolinic |
| | acid, prepared in same fashion as |
| | Compound 13 |

### Example E

### SYNTHESIS of Compound 44

Preparation of N-(1-acetylpiperidin-4-yl)-6-(1H-imidazol-1-yl)picolinamide (Compound 44)

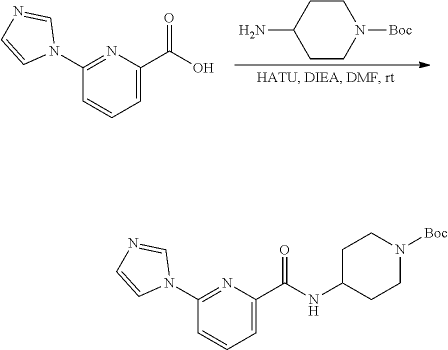 [SMILES: O=C(O)c1cccc(-n2ccnc2)n1 ; CC(C)(C)OC(=O)N1CCC(N)CC1 ; CC(C)(C)OC(=O)N1CCC(NC(=O)c2cccc(-n3ccnc3)n2)CC1]

Step 1: Preparation of tert-Butyl 4-(6-(1H-imidazol-1-yl)picolinamido)piperidine-1-carboxylate. To a 50-mL round-bottom flask was added DMF (5 mL), 6-(imidazol-1-yl)pyridine-2-carboxylic acid (200 mg, 1.06 mmol), DIEA (273 mg, 2.11 mmol), HATU (603 mg, 1.59 mmol) and tert-butyl 4-aminopiperidine-1-carboxylate (212 mg, 1.06 mmol). The resulting solution was stirred for 2 h at r.t. and quenched with 50 mL of water. The mixture was extracted with ethyl acetate (3×50 mL), the organic layers were combined, washed with brine, dried over sodium sulfate and concentrated. The product was purified with silica gel using ethyl acetate/petroleum ether (1:2) to give tert-butyl 4-[6-(imidazol yl)pyridine-2-amido]piperidine-1-carboxylate (200 mg, 0.54 mmol, 51%) as a yellow solid.

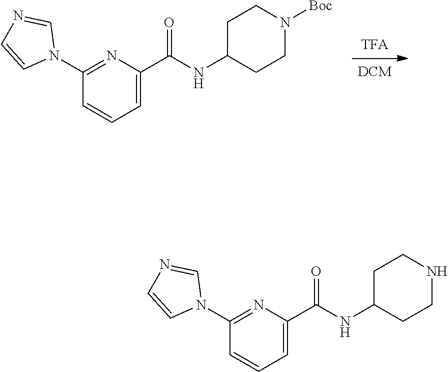 [SMILES: CC(C)(C)OC(=O)N1CCC(NC(=O)c2cccc(-n3ccnc3)n2)CC1 ; O=C(NC1CCNCC1)c1cccc(-n2ccnc2)n1]

Step 2: Preparation of 6-(1H-imidazol-1-yl)-N-(piperidin-4-yl)picolinamide. To a 50-mL round-bottom flask was added DCM (5 mL), tert-butyl 4-[6-(imidazol-1-yl)pyridine-2-amido]piperidine-1-carboxylate (200 mg, 0.54 mmol) and TFA (0.5 mL). The resulting solution was stirred for 30 min at r.t. and concentrated under reduced pressure to provide 6-(1H-imidazol-1-yl)-N-(piperidin-4-yl)picolinamide (146 mg, 0.54 mmol, 100%) as a glassy solid.

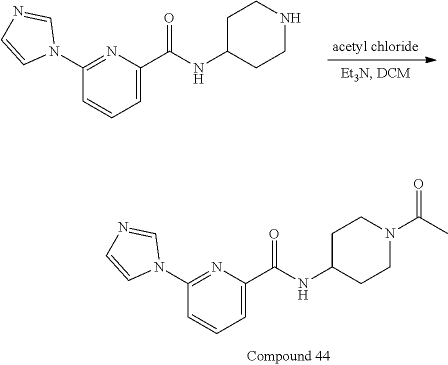 [SMILES: O=C(NC1CCNCC1)c1cccc(-n2ccnc2)n1 ; CC(=O)N1CCC(NC(=O)c2cccc(-n3ccnc3)n2)CC1]

Step 3: Preparation of N-(1-acetylpiperidin-4-yl)-6-(1H-imidazol-1-yl)picolinamide. To a 25 mL round-bottom flask was added DCM (5 mL), 6-(imidazol-1-yl)-N-(piperidin-4-yl)pyridine-2-carboxamide (100 mg, 0.369 mmol), Et₃N (112 mg, 1.11 mmol) and acetyl chloride (29 mg, 0.369 mmol). The resulting solution was stirred for 2 h at r.t., quenched with water (30 mL) and extracted with DCM (3×30 mL). The organic phases were combined, washed with brine, dried over sodium sulfate, concentrated under reduced pressure and purified using reverse phase HPLC with the following conditions: Waters X select column CSH OBD Column 30*150 mm Sum; mobile phase, Water (10 MMOL/L NH₄HCO₃+0.1% NH₃.H₂O) and ACN (35% Phase B up to 65% in 8 min) to provide N-(1-acetylpiperidin-4-yl)-6-(imidazol-1-yl)pyridine-2-carboxamide (20 mg, 0.064 mmol, 17%) as a white solid. LRMS (APCI) m/z 314 (M+H). ¹H NMR (300 MHz, CDCL3) δ8.33 (s, 1H), 8.17 (dd, J=7.6, 0.9 Hz, 1H), 8.04 (t, J=7.9 Hz, 1H), 7.68 (d, J=8.3 Hz, 1H), 7.62 (s, 1H), 7.53 (dd, J=8.1, 0.9 Hz, 1H), 4.60 (d, J=13.7 Hz, 1H), 3.87 (d, J=13.6 Hz, 1H), 3.34-3.19 (m, 1H), 2.91-2.77 (m, 1H), 2.18-2.00 (m, 5H), 1.73-1.35 (m, 4H).

Compounds 45-54, 57, and 58 were prepared using the methods provided in the table below.

| Compound | |
|---|---|
| Number | Method of Preparation |
| 45 | Prepared in same fashion as Compound 44 |
| 46 | Beginning with 6-(1H-imidazol-1-yl)picolinic acid, |
| | amide bond formation as |
| | Compound 44 |
| 47 | Prepared in same fashion as Compound 44 |
| 48 | Prepared in same fashion as Compound 44 |
| 49 | Beginning with 6-(1H-imidazol-1-yl)picolinic |
| | acid, amide bond formation as |
| | Compound 44 |
| 50 | Prepared in same fashion as Compound 11 |
| | with amide bond formation as |
| | Compound 44 |
| 51 | Prepared in same fashion as Compound 11 |
| | with amide bond formation as |
| | Compound 44 |
| 52 | Prepared in same fashion as Compound 11 |
| | with amide bond formation as |
| | Compound 44 |
| 53 | Beginning with 6-bromo-5-fluoropicolinic |
| | acid, prepared in same fashion as |
| | Compound 13 |
| 54 | Beginning with 6-bromo-5-fluoropicolinic |
| | acid, prepared in same fashion as |
| | Compound 13 |
| 57 | Prepared in same fashion as Compound 12 |
| 58 | Prepared in same fashion as Compound 12 |

### Example F

### Synthesis of Compound 59

### Preparation of 6-(1-Methyl-1H-imidazol-5-yl)-N-(pyridin-3-yl)picolinamide (Compound 59)

Step 1: Preparation of 6-bromo-N-(pyridin-3-yl)picolinamide. Beginning with 6-bromopicolinic acid, amide bond formation was performed as in the synthesis of Compound 17.

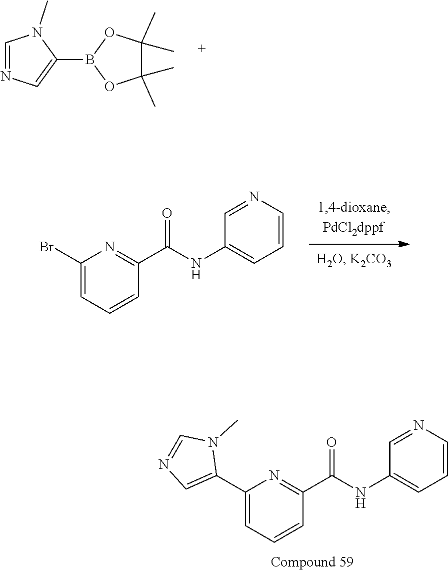 [SMILES: Cn1cncc1B1OC(C)(C)C(C)(C)O1 ; O=C(Nc1cccnc1)c1cccc(Br)n1 ; Cn1cncc1-c1cccc(C(=O)Nc2cccnc2)n1]

Step 2: Preparation of 6-(1-Methyl-1H-imidazol-5-yl)-N-(pyridin-3-yl)picolinamide. 1-methyl-2-(4,4,5,5-tetramethyl-1,3,2-dioxaborolan-2-yl)-1H-imidazole (44 mg, 0.211 mmol) was combined with 6-bromo-N-(pyridin-3-yl)picolinamide (49 mg, 0.176 mmol), PdCl₂dppf (25 mg, 0.035 mmol) and K₂CO₃ (73 mg, 0.529 mmol). 1,4-dioxane (2 mL) was added followed by H₂O (0.5 mL) and the resulting mixture was heated in a microwave at 130° C. for 20 min. The solvents were evaporated under reduced pressure and the product was purified using reverse phase HPLC with a 40 minute gradient from 5-100% ACN/water (Phenomenex Gemini 5 micron C18 Axia pack 150×21.2 mm column) to give 6-(1-methyl-1H-imidazol-5-yl)-N-(pyridin-3-yl)picolinamide (28 mg, 0.100 mmol, 57%) as a white solid. LRMS (APCI) m/z 280.1 (M+H). ¹H NMR (400 MHz, Methanol-d₄) δ 8.99 (s, 1H), 8.34 (d, J=7.9 Hz, 2H), 8.17-7.58 (m, 5H), 7.51-7.42 (m, 1H), 4.15 (s, 3H).

Compounds 60-64 were prepared using the methods provided in the table below.

| Compound | |
|---|---|
| Number | Method of Preparation |
| 60 | Beginning with 6-bromo-4-(trifluoromethyl)picolinic |
| | acid, prepared in same fashion as Compound 13 |
| 61 | Beginning with 6-bromo-4-(trifluoromethyl)picolinic |
| | acid, prepared in same fashion as Compound 13 |
| 62 | Beginning with 6-bromo-4-(trifluoromethyl)picolinic |
| | acid, prepared in same fashion as Compound 13 |
| 63 | Prepared in same fashion as Compound 12 |
| 64 | Prepared in same fashion as Compound 12 |
| 65 | Amide bond formation as Compound 13 followed |
| | by Suzuki coupling as Compound 59 |
| 71 | Amide bond formation as Compound 13 followed |
| | by Suzuki coupling as Compound 59 |
| 77 | Amide bond formation as Compound 13 followed |
| | by Suzuki coupling as Compound 59 |

### Example G

### Synthesis of Compounds 66 and 76

### Preparation of 6-(5-carbamoyl-1H-imidazol-1-yl)-N-(6-(trifluoromethyl)pyridin-3-yl)picolinamide and 6-(4-carbamoyl-1H-imidazol-1-yl)-N-(6-(trifluoromethyl)pyridin-3-yl)picolinamide (Compounds 66 and 76)

Step 1: Preparation of 6-bromo-N-(6-(trifluoromethyl)pyridin-3-yl)picolinamide. Beginning with 6-bromopicolinic acid, amide bond formation was performed as in the synthesis of Compound 13.

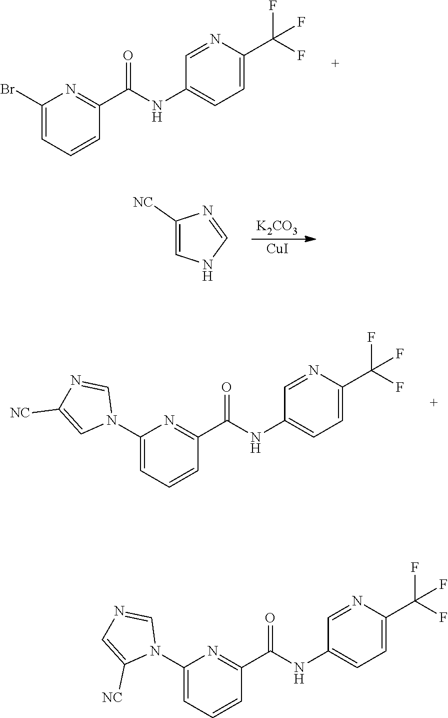

Step 2: Preparation of 6-(4-Cyano-1H-imidazol-1-yl)-N-(6-(trifluoromethyl)pyridin-3-yl)picolinamide and 6-(5-cyano-1H-imidazol-1-yl)-N-(6-(trifluoromethyl)pyridin yl)picolinamide 6-Bromo-N-(6-(trifluoromethyl)pyridin-3-yl)picolinamide (150 mg, 0.433 mmol) was combined with 1H-imidazole-4-carbonitrile (61 mg, 0.650 mmol), K₂CO₃ (181 mg, 1.30 mmol) and CuI (41 mg, 0.217 mmol). To the solids was added DMF (4 mL) and the mixture was heated in a microwave at 130° C. for 20 min. The reaction was diluted with ethyl acetate (20 mL) and water (20 mL) and filtered through celite. Additional ethyl acetate (75 mL) and water (20 mL) was added and the layers were shaken and separated. The organic phase was washed with brine, dried over sodium sulfate, concentrated in vacuo and purified using reverse phase HPLC with a 40 minute gradient from 5-100% ACN/water (Phenomenex Gemini 5 micron C18 Axia pack 150×21.2 mm column) to provide a mixture of 6-(4-cyano-1H-imidazol-1-yl)-N-(6-(trifluoromethyl)pyridin-3-yl)picolinamide and 6-(5-cyano-1H-imidazol-1-yl)-N-(6-(trifluoromethyl)pyridin-3-yl)picolinamide (40 mg, 0.112 mmol, 26%) as a white solid that was used in the next step without additional purification.

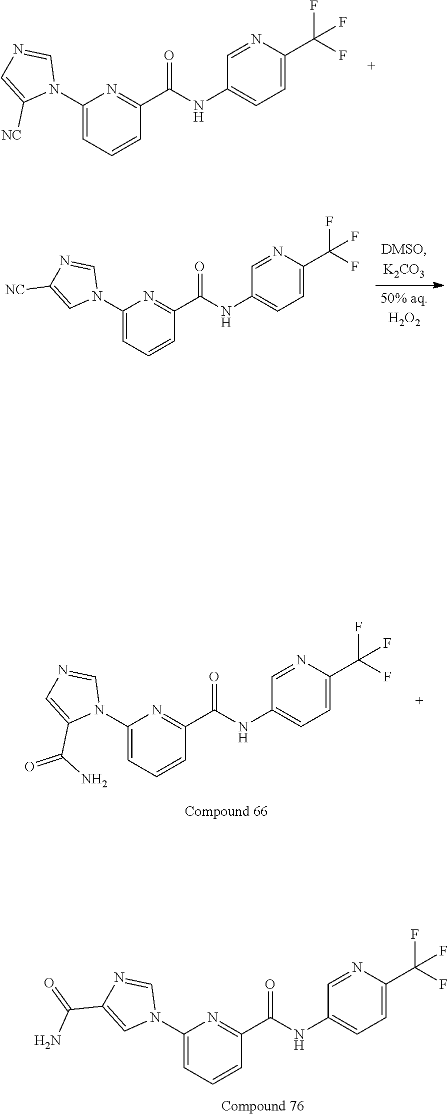 [SMILES: N#Cc1cncn1-c1cccc(C(=O)Nc2ccc(C(F)(F)F)nc2)n1 ; N#Cc1cn(-c2cccc(C(=O)Nc3ccc(C(F)(F)F)nc3)n2)cn1 ; NC(=O)c1cncn1-c1cccc(C(=O)Nc2ccc(C(F)(F)F)nc2)n1 ; NC(=O)c1cn(-c2cccc(C(=O)Nc3ccc(C(F)(F)F)nc3)n2)cn1]

Step 3: Preparation of 6-(5-carbamoyl-1H-imidazol-1-yl)-N-(6-(trifluoromethyl)pyridin-3-yl)picolinamide and -(4-carbamoyl-1H-imidazol-1-yl)-N-(6-(trifluoromethyl)pyridin-3-yl)picolinamide (Compounds 66 and 76). A mixture of 6-(4-cyano-1H-imidazol-1-yl)-N-(6-(trifluoromethyl)pyridin-3-yl)picolinamide and 6-(5-cyano-1H-imidazol-1-yl)-N-(6-(trifluoromethyl)pyridin-3-yl)picolinamide (30 mg, 0.0.084 mmol) was combined with K₂CO₃ (35 mg, 0.251 mmol) and DMSO (1.5 mL). 50% aq. H₂O₂ (57 μL, 0.840 mmol) was added and the suspension was stirred at r.t. for 3 h. It was diluted with MeOH (4 mL) and water (2 mL) and filtered. The filtered, white solid was dissolved in DMSO with gently heating and purified using reverse phase HPLC with a 40 minute gradient from 5-100% ACN/water (Phenomenex Gemini 5 micron C18 Axia pack 150×21.2 mm column) to provide 6-(4-carbamoyl-1H-imidazol-1-yl)-N-(6-(trifluoromethyl)pyridin-3-yl)picolinamide (10 mg, 0.026 mmol) as a white solid and 6-(5-carbamoyl-1H-imidazol-1-yl)-N-(6-(trifluoromethyl)pyridin-3-yl)picolinamide (3 mg, 0.008 mmol) as a white solid. Characterization data for 6-(4-carbamoyl-1H-imidazol-1-yl)-N-(6-(trifluoromethyl)pyridin-3-yl)picolinamide LRMS (APCI) m/z 377.0 (M+H). ¹H NMR (400 MHz, DMSO-d₆) δ 11.04 (s, 1H), 9.23 (d, J=2.3 Hz, 1H), 9.03 (s, 1H), 8.95 (s, 1H), 8.57 (dd, J=8.6, 2.4 Hz, 1H), 8.37-8.21 (m, 2H), 8.17 (d, J=7.3 Hz, 1H), 8.00 (d, J=8.6 Hz, 1H), 7.55 (s, 1H), 7.32 (s, 1H). Characterization data for 6-(5-carbamoyl-1H-imidazol-1-yl)-N-(6-(trifluoromethyl)pyridin-3-yl)picolinamide LRMS (APCI) m/z 377.0 (M+H). ¹H NMR (400 MHz, Methanol-d₄) δ 9.12 (s, 1H), 8.68 (s, 1H), 8.60 (d, J=8.3 Hz, 1H), 8.34 (d, J=7.6 Hz, 1H), 8.24 (t, J=7.8 Hz, 1H), 7.90-7.76 (m, 3H).

Compounds 95-101, 132, and 133 were prepared using the methods provided in the table below.

| Compound | |
|---|---|
| Number | Method of Preparation |
| 95 | Prepared in same fashion as Compound 13 |
| 96 | Beginning with 6-bromo-4-methylpicolinic acid, |
| | prepared in same fashion as Compound 13 |
| 97 | Beginning with 6-bromo-4-methylpicolinic acid, |
| | prepared in same fashion as Compound 13 |
| 98 | Beginning with 6-bromo-4-methylpicolinic |
| | acid, amide bond formation as for |
| | Compound 13 followed by Suzuki |
| | coupling as for Compound 59 |
| 99 | Beginning with 6-bromo-4-methylpicolinic |
| | acid, amide bond formation as for |
| | Compound 13 followed by Suzuki |
| | coupling as for Compound 59 |
| 100 | Beginning with 6-bromo-3-methylpicolinic |
| | acid, prepared in same fashion as |
| | Compound 13 |
| 101 | Beginning with 6-bromo-3-methylpicolinic |
| | acid, prepared in same fashion as |
| | Compound 13 |
| 132 | Beginning with 6-bromo-4-methylpicolinic |
| | acid, amide bond formation as for |
| | Compound 13 followed by Stille coupling |
| | as for Compound 11 |
| 133 | Beginning with 6-bromo-4-methylpicolinic |
| | acid, amide bond formation as for |
| | Compound 13 followed by Stille coupling |
| | as for Compound 11 |

### Example H

### Synthesis of Compound 134

### Synthesis of N-((1r,4r)-4-(2-Hydroxypropan-2-yl)cyclohexyl)-6-(1H-imidazol-1-yl)picolinamide

(Compound 134)

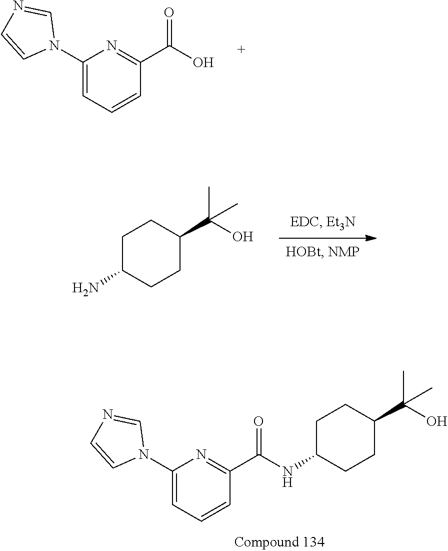 [SMILES: O=C(O)c1cccc(-n2ccnc2)n1 ; CC(C)(O)[C@H]1CC[C@H](N)CC1 ; CC(C)(O)[C@H]1CC[C@H](NC(=O)c2cccc(-n3ccnc3)n2)CC1]

Preparation of N-((1r,4r)-4-(2-Hydroxypropan-2-yl)cyclohexyl)-6-(1H-imidazol-1-yl)picolinamide (Compound 134). 6-(1H-imidazol-1-yl)picolinic acid (60 mg, 0.317 mmol) was combined with N-(3-dimethylaminopropyl)-N′-ethylcarbodiimide hydrochloride (122 mg, 0.634 mmol), HOBt (43 mg, 0.317 mmol), NMP (1 mL) and triethylamine (133 mL, 0.952 mmol). The mixture was stirred at r.t. for 15 min and 2-((1r,4r)-4-aminocyclohexyl)propan-2-ol (60 mg, 0.381 mmol) was added and stirred at 70° C. for 18 h. The product was purified using reverse phase HPLC with a 40 minute gradient from 5-100% ACN/water (Phenomenex Gemini 5 micron C18 Axia pack 150×21.2 mm column) to provide N-((1r,4r)-4-(2-hydroxypropan-2-yl)cyclohexyl)-6-(1H-imidazol-1-yl)picolinamide (33 mg, 0.099 mmol, 31%) as a white solid. LRMS (APCI) m/z 329.1 (M+H). ¹H NMR (400 MHz, Methanol-d₄) δ 8.85 (s, 1H), 8.19-8.04 (m, 3H), 7.89 (d, J=8.1 Hz, 1H), 7.20 (s, 1H), 3.95-3.82 (m, 1H), 2.01 (dd, J=32.3, 12.2 Hz, 4H), 1.53 (q, J=12.2, 11.7 Hz, 2H), 1.44-1.20 (m, 3H), 1.18 (s, 6H).

Compounds 1-6, 8, 10, 135, and 136 were prepared using the methods provided in the table below.

| Compound | |
|---|---|
| Number | Method of Preparation |
| 1 | Beginning with 4-(1H-imidazol-1-yl)picolinic |
| | acid, prepared in same fashion as Compound 134 |
| 2 | Beginning with 4-(1H-imidazol-1-yl)picolinic |
| | acid, prepared in same fashion as Compound 134 |
| 3 | Beginning with 4-(1H-imidazol-1-yl)picolinic |
| | acid, prepared in same fashion as Compound 134 |
| 4 | Beginning with 4-(1H-imidazol-1-yl)picolinic |
| | acid, prepared in same fashion as Compound 134 |
| 5 | Beginning with 4-(1H-imidazol-1-yl)picolinic |
| | acid, prepared in same fashion as Compound 134 |
| 6 | Beginning with 4-(1H-imidazol-1-yl)picolinic |
| | acid, prepared in same fashion as Compound 134 |
| 8 | Prepared in same fashion as Compound 134 |
| 10 | Beginning with 2-(1H-imidazol- l-yl)isonicotinic |
| | acid, prepared in same |
| | fashion as Compound 134 |
| 135 | Prepared in same fashion as Compound 134 |
| 136 | Prepared in same fashion as Compound 134 |

### Example I

### Synthesis of Compound 9

### Preparation of 3-(1H-imidazol-1-yl)-N-(pyridin-3-yl)benzamide (Compound 9)

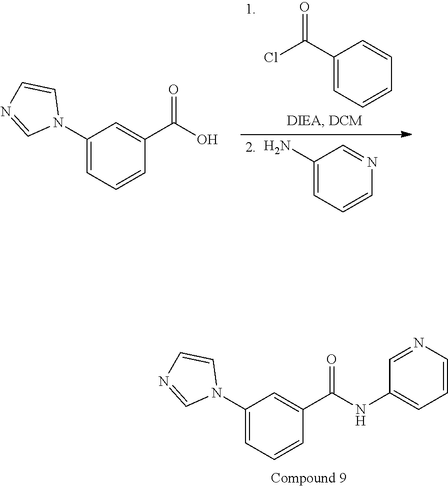

Preparation of 3-(1H-imidazol-1-yl)-N-(pyridin-3-yl)benzamide (Compound 9). To a solution of 3-(1H-imidazol-1-yl)benzoic acid (53.5 mg, 0.28 mmol) and DIEA (0.15 mL, 0.85 mmol) in DCM (3 mL) was added benzoyl chloride (0.04 mL, 0.34 mmol) dropwise and stirred for 30 min. Next, 3-aminopyridine (80.3 mg, 0.85 mmol) was added, stirred at rt for 30 min, concentrated, and directly purified using reverse phase HPLC with a 50 minute gradient from 0-100% ACN/water (Phenomenex Gemini 5 micron C18 Axia pack 150×21.2 mm column) to yield 3-(1H-imidazol-1-yl)-N-(pyridin-3-yl)benzamide (2.0 mg, 0.01 mmol, 3%). LRMS (ESI) m/z 265.1 (M+H). ¹H NMR (400 MHz, DMSO-d₆) δ 10.55 (s, 1H), 8.94 (d, J=2.5 Hz, 1H), 8.37 (s, 1H), 8.34 (d, J=4.7 Hz, 1H), 8.22-8.18 (m, 2H), 7.92 (t, J=8.9 Hz, 2H), 7.86 (s, 1H), 7.70 (t, J=8.0 Hz, 1H), 7.43 (dd, J=8.3, 4.7 Hz, 1H), 7.16 (s, 1H).

Compounds 7 and 143 were prepared using the methods provided in the table below.

| Compound Number | Method of Preparation |
|---|---|
| 7 | Prepared in same fashion as Compound 9 |
| 143 | Prepared in same fashion as Compound 13 |

### Example J

### Synthesis of Compound 163

### Preparation of N-(6-(Difluoromethyl)pyridin-3-yl)-4-methoxy-6-(thiazol-5-yl)picolinamide (Compound 163)

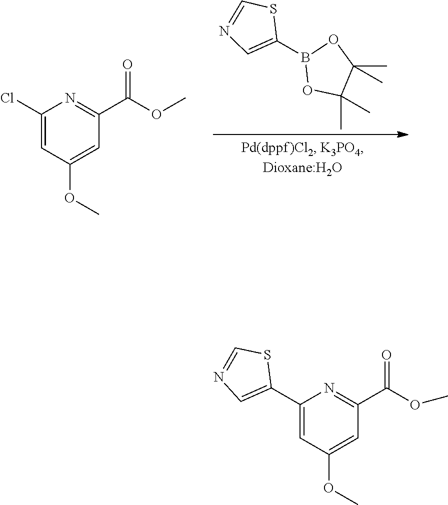 [SMILES: COC(=O)c1cc(OC)cc(Cl)n1 ; CC1(C)OB(c2cncs2)OC1(C)C ; COC(=O)c1cc(OC)cc(-c2cncs2)n1]

Step 1: Preparation of Methyl 4-methoxy-6-(thiazol-5-yl)picolinate. To a stirred solution of methyl 6-chloro-4-methoxypyridine-2-carboxylate (200 mg, 0.992 mmol) in dioxane (2 mL) at r.t. were added 5-(4,4,5,5-tetramethyl-1,3,2-dioxaborolan-2-yl)-1,3-thiazole (230 mg, 1.090 mmol), Pd(dppf)Cl₂CH₂Cl₂ (160 mg, 0.196 mmol), K₃PO₄ (420 mg, 1.979 mmol) in H₂O (0.2 mL) and 600 mg 4A MS. The resulting mixture was stirred at 120° C. for 18 h under a nitrogen atmosphere. The reaction was cooled to r.t., filtered and the filter cake was washed twice with MeOH (10 mL). The filtrate was concentrated under reduced pressure and purified by C18 column chromatography using water (0.05% NH₄HCO₃): ACN=1:1 as the mobile phase to afford methyl 4-methoxy-6-(thiazol-5-yl)picolinate (160 mg, 0.64 mmol, 65%) as a beige solid. LRMS (ESI) m/z 251 (M+H).

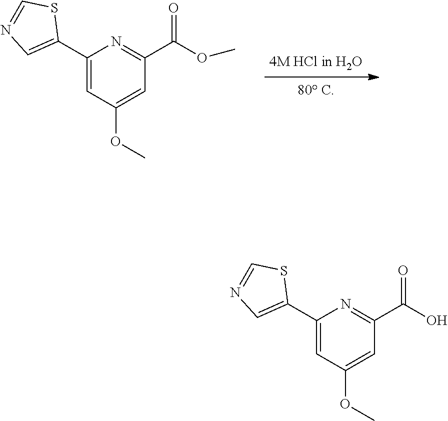 [SMILES: COC(=O)c1cc(OC)cc(-c2cncs2)n1 ; COc1cc(C(=O)O)nc(-c2cncs2)c1]

Step 2: Preparation of 4-methoxy-6-(thiazol-5-yl)picolinic acid. To methyl 4-methoxy-6-(thiazol-5-yl)picolinate (140 mg, 0.56 mmol) was added HCl (3 mL of 4 M in H₂O) and the resulting mixture was stirred at 80° C. for 18. The reaction was cooled to r.t. and concentrated in vacuo to afford 4-methoxy-6-(thiazol-5-yl)picolinic acid (132 mg, 0.56 mmol, 100%) as a beige solid which was used in the subsequent step without additional purification. LRMS (ES) m/z 237 (M+H).

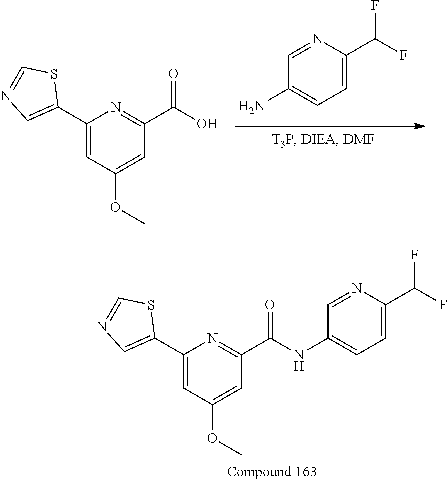 [SMILES: COc1cc(C(=O)O)nc(-c2cncs2)c1 ; Nc1ccc(C(F)F)nc1 ; COc1cc(C(=O)Nc2ccc(C(F)F)nc2)nc(-c2cncs2)c1]

Step 3: Preparation of N-(6-(Difluoromethyl)pyridin-3-yl)-4-methoxy-6-(thiazol-5-yl)picolinamide (Compound 163). To a solution of 4-methoxy-6-(thiazol-5-yl)picolinic acid (100 mg, 0.423 mmol) in DMF (2 mL) at r.t. were added 6-(difluoromethyl)pyridin-3-amine (61 mg, 0.423 mmol), T₃P (404 mg, 0.635 mmol) and DIEA (164 mg, 1.269 mmol). The resulting mixture was stirred at r.t. for 18 h and purified by C18 column chromatography using water (0.05% NH₄HCO₃):ACN=1:1 as the mobile phase to afford N-(6-(difluoromethyl)pyridin-3-yl)-4-methoxy-6-(thiazol-5-yl)picolinamide (52 mg, 0.143 mmol, 34%) as a dark grey solid. LRMS (ES) m/z 363 (M+H). ¹H NMR (300 MHz, DMSO-d₆) δ 10.70 (s, 1H), 9.24 (s, 1H), 9.12 (s, 1H), 8.88 (s, 1H), 8.48 (d, J=10.0 Hz, 1H), 7.76 (dd, J=5.1, 3.1 Hz, 2H), 7.59 (d, J=1.9 Hz, 1H), 6.95 (t, J=55.1 Hz, 1H), 4.01 (s, 3H).

### Example K

### Synthesis of Compound 166

### Preparation of 6-(1H-imidazol-1-yl)-4-methoxy-N-((1r,4r)-4-methylcyclohexyl)picolinamide (Compound 166)

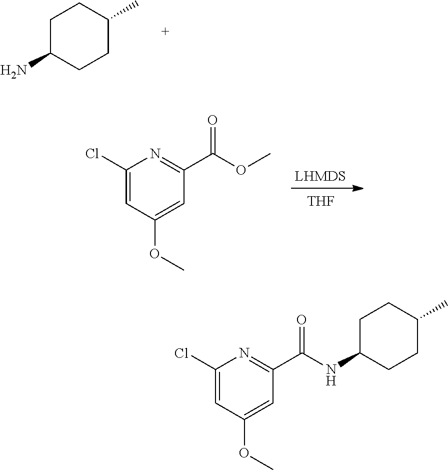

Step 1: Preparation of 6-Chloro-4-methoxy-N-((1r,4r)-4-methylcyclohexyl)picolinamide. To a stirred solution of (1r,4r)-4-methylcyclohexan-1-amine (84 mg, 0.744 mmol) in THF (3 mL) at 0° C. was added LHMDS (1.1 mL, 1.116 mmol) dropwise over 5 min After stirring 30 min, methyl 6-chloro-4-methoxypyridine carboxylate (150 mg, 0.744 mmol) in THF (1 mL) was added. The resulting mixture was stirred at r.t. for 2 h, quenched with MeOH, concentrated under reduced pressure and purified by C18 column chromatography using water (0.05% NH₄HCO₃): ACN=1:4 as the mobile phase to afford 6-Chloro-4-methoxy-N-((1r,4r)-4-methylcyclohexyl)picolinamide (170 mg, 0.60 mmol, 81%) as a white solid. LRMS (ES) m/z 283 (M+H).

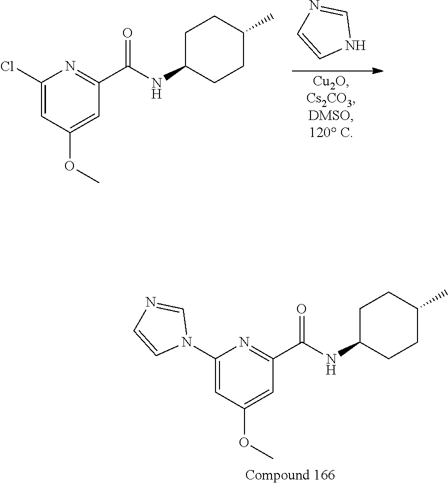

Step 2: Preparation of 6-(1H-imidazol-1-yl)-4-methoxy-N-((1r,4r)-4-methylcyclohexyl)picolinamide. To a solution of 6-chloro-4-methoxy-N-((1r,4r)-4-methylcyclohexyl)picolinamide (90 mg, 0.318 mmol) in DMSO (3 mL) at r.t. were added imidazole (26 mg, 0.382 mmol), Cu₂O (5 mg, 0.035 mmol) and Cs₂CO₃ (208 mg, 0.638 mmol). The resulting mixture was stirred at 120° C. for 18 h, cooled to r.t. and purified by C18 column chromatography using water (0.05% NH₄HCO₃): ACN=4:1 as the mobile phase to obtain 6-(1H-imidazol-1-yl)-4-methoxy-N-((1r,4r)-4-methylcyclohexyl)picolinamide (18 mg, 0.057 mmol, 18%) as an off-white solid. LRMS (ES) m/z 315 (M+H).

### Example L

### Synthesis of Compound 179

### Preparation of N-(6-(Difluoromethyl)pyridin-3-yl)-4-(pyrrolidin-1-yl)-6-(thiazol-5-yl)picolinamide (Compound 179)

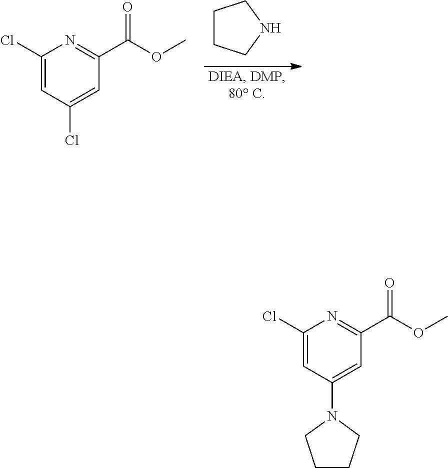

Step 1: Preparation of methyl 6-chloro-4-(pyrrolidin-1-yl)picolinate. To a stirred solution of methyl 4,6-dichloropyridine-2-carboxylate (3.0 g, 14.5 mmol) in NMP (30 mL) at r.t. were added pyrrolidine (1.01 g, 14.2 mmol) and DIEA (3.78 g, 29.2 mmol). The resulting mixture was stirred at 80° C. for 18 h. The mixture was cooled to r.t. and purified by C18 column chromatography using water (0.05% NH₄HCO₃): ACN=1:1 as the mobile phase to afford methyl 6-chloro-4-(pyrrolidin-1-yl)picolinate (2.4 g, 10.0 mmol, 69%) as a yellow solid and 540 mg of the undesired regioisomer confirmed by NOESY. LRMS (ES) m/z 251 (M+H).

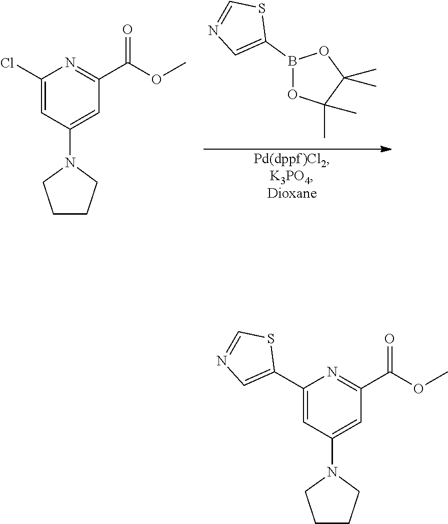

Step 2: Preparation of methyl 4-(pyrrolidin-1-yl)-6-(thiazol-5-yl)picolinate. Prepared using the same Suzuki coupling procedure as Compound 163.

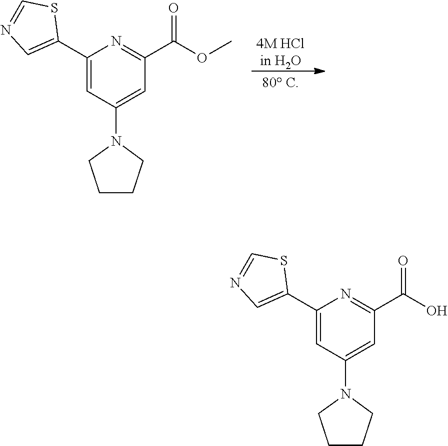 [SMILES: COC(=O)c1cc(N2CCCC2)cc(-c2cncs2)n1 ; "4M HCl in H2O" ; "80° C." ; O=C(O)c1cc(N2CCCC2)cc(-c2cncs2)n1]

Step 3: Preparation of 4-(Pyrrolidin-1-yl)-6-(thiazol-5-yl)picolinic acid. Prepared using the same ester hydrolysis procedure as Compound 163.

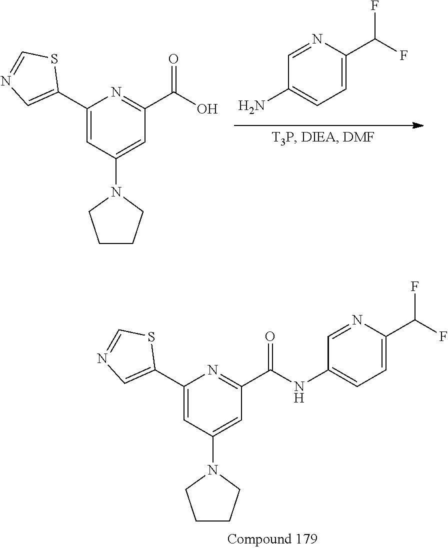

Step 4: Preparation of N-(6-(Difluoromethyl)pyridin-3-yl)-4-(pyrrolidin-1-yl)-6-(thiazol-5-yl)picolinamide. Prepared using the same amide bond formation procedure as Compound 165. LRMS (ES) m/z 402 (M+H). ¹H NMR (300 MHz, DMSO-d₆) δ 10.61 (s, 1H), 9.17 (s, 1H), 9.15-9.07 (m, 1H), 8.79 (s, 1H), 8.53-8.42 (m, 1H), 7.75 (d, J=8.5 Hz, 1H), 7.20-6.73 (m, 3H), 3.56-3.41 (m, 4H), 2.11-1.87 (m, 4H).

Compound 180 was prepared in the same fashion as Compound 179 except with Cu₂O coupling as for Compound 166.

### Example M

### Synthesis of Compound 36

### Preparation of 4-(1H-imidazol-1-yl)-N-(pyridin-3-yl)pyrimidine-2-carboxamide (Compound 36)

Step 1: Preparation of 4-chloro-N-(pyridin-3-yl)pyrimidine-2-carboxamide. Beginning with 4-chloropyrimidine-2-carboxylic acid, amide bond formation was performed as in Compound 13.

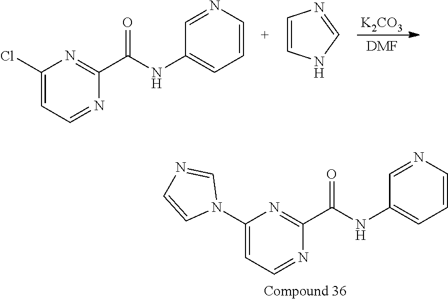

Step 2: Preparation of 4-(1H-imidazol-1-yl)-N-(pyridin-3-yl)pyrimidine-2-carboxamide. 4-Chloro-N-(pyridin-3-yl)pyrimidine-2-carboxamide (69 mg, 0.294 mmol) was combined with 1H-imidazole (60 mg, 0.882 mmol), K₂CO₃ (123 mg, 0.882 mmol) and DMF (3 mL). The mixture was heated in an oil bath at 100° C. for 30 min., cooled to r.t., filtered through a syringe filter and purified using reverse phase HPLC with a 40 minute gradient from 5-100% ACN/water (Phenomenex Gemini 5 micron C18 Axia pack 150×21.2 mm column) to give 4-(1H-imidazol-1-yl)-N-(pyridin-3-yl)pyrimidine-2-carboxamide (35 mg, 0.131 mmol, 45%) as a white solid. LRMS (APCI) m/z 267.1 (M+H). ¹H NMR (400 MHz, DMSO-d₆) δ 10.94 (s, 1H), 9.14 (d, J=5.6 Hz, 1H), 9.03 (s, 1H), 8.98 (s, 1H), 8.38 (d, J=4.7 Hz, 1H), 8.31-8.25 (m, 2H), 8.15 (d, J=5.7 Hz, 1H), 7.49-7.40 (m, 1H), 7.25 (s, 1H).

Compounds 37, 67-70, 72-75, 78-87, 93, 94, 106, and 107 were prepared using the methods provided in the table below.

| Compound Number | Method of Preparation |
|---|---|
| 37 | Beginning with 2-chloropyrimidine-4-carboxylic |
| | acid, amide bond formation as for Compound 13 |
| | followed by nucleophilic aromatic substitution |
| | as for Compound 36 |
| 67 | Beginning with 2-chloropyrimidine-4-carboxylic |
| | acid, amide bond formation as for Compound 13 |
| | followed by nucleophilic aromatic substitution |
| | as for Compound 36 |
| 68 | Beginning with 2-chloropyrimidine-4-carboxylic |
| | acid, amide bond formation as for Compound 13 |
| | followed by nucleophilic aromatic substitution |
| | as for Compound 36 |
| 69 | Beginning with 2-chloropyrimidine-4-carboxylic |
| | acid, amide bond formation as for Compound 13 |
| | followed by Suzuki coupling as for Compound 59 |
| 70 | Beginning with 2-chloropyrimidine-4-carboxylic |
| | acid, amide bond formation as for Compound 13 |
| | followed by Suzuki coupling as for Compound 59 |
| 72 | Prepared in same fashion as Compound 36 |
| 73 | Prepared in same fashion as Compound 36 |
| 74 | Beginning with 4-chloropyrimidine-2-carboxylic |
| | acid, amide bond formation as for Compound 13 |
| | followed by Suzuki coupling as for Compound 59 |
| 75 | Prepared in same fashion as Compound 74 |
| 78 | Prepared in same fashion as Compound 37 |
| 79 | Prepared in same fashion as Compound 37 |
| 80 | Prepared in same fashion as Compound 37 |
| 81 | Prepared in same fashion as Compound 37 |
| 82 | Prepared in same fashion as Compound 69 |
| 83 | Prepared in same fashion as Compound 69 |
| 84 | Prepared in same fashion as Compound 69 |
| 85 | Prepared in same fashion as Compound 69 |
| 86 | Beginning with 2-chloropyrimidine-4-carboxylic |
| | acid, amide bond formation as for Compound 13 |
| | followed by Stille coupling as for Compound 11 |
| 87 | Prepared in same fashion as Compound 86 |
| 93 | Prepared in same fashion as Compound 37 |
| 94 | Prepared in same fashion as Compound 69 |
| 106 | Prepared in same fashion as Compound 37 |
| 107 | Prepared in same fashion as Compound 69 |

### Example N

### Synthesis of Compound 108

### Preparation of 6-Cyclopropyl-N-((1r,4r)-4-methoxycyclohexyl)-2-(thiazol-5-yl)pyrimidine-4-carboxamide (Compound 108)

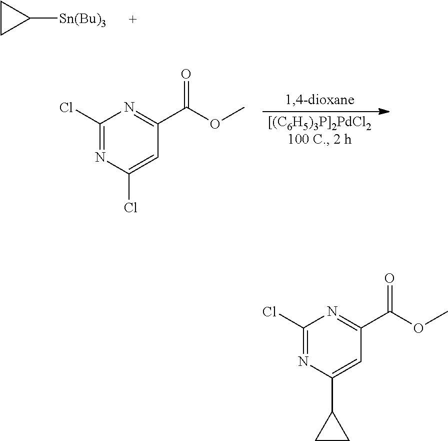

Step 1: Preparation of methyl 2-chloro-6-cyclopropylpyrimidine-4-carboxylate. Methyl 2,6-dichloropyrimidine-4-carboxylate (500 mg, 2.42 mmol) was combined with tributyl(cyclopropyl)stannane (880 mg, 2.66 mmol), trans-dichlorobis(triphenylphosphine)palladium(II) (170 mg, 0.242 mmol) and 1,4-dioxane (10 mL). The mixture was heated in an oil bath at 100° C. for 2 h. The solvent was evaporated in vacuo and the product was purified with silica gel using 15% ethyl acetate/hexanes to give methyl 2-chloro-6-cyclopropylpyrimidine-4-carboxylate (255 mg, 1.199 mmol, 50%) as a white solid. LRMS (APCI) m/z 213.0 (M+H).

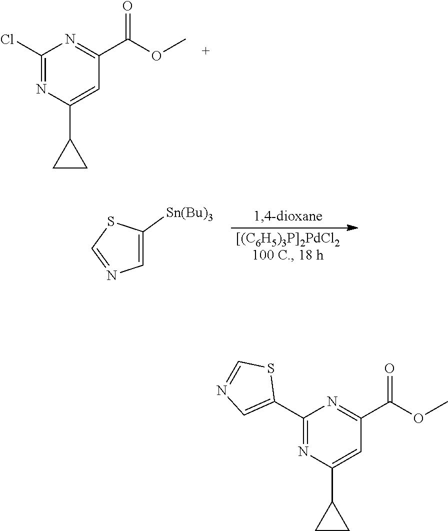

Step 2: Preparation of methyl 6-cyclopropyl-2-(thiazol-5-yl)pyrimidine-4-carboxylate. Methyl 2-chloro-6-cyclopropylpyrimidine-4-carboxylate (255 mg, 1.20 mmol) was combined with 5-(tributylstannyl)thiazole (494 mg 1.32 mmol), trans-dichlorobis(triphenylphosphine)palladium(II) (84 mg, 0.120 mmol) and 1,4-dioxane (7 mL). The mixture was heated in an oil bath at 100° C. for 18 h. The solvent was evaporated and the product was purified with silica gel using 30% ethyl acetate/hexanes providing methyl 6-cyclopropyl-2-(thiazol-5-yl)pyrimidine-4-carboxylate (255 mg, 0.976 mmol, 81%) as a white solid. LRMS (APCI) m/z 262.0 (M+H).

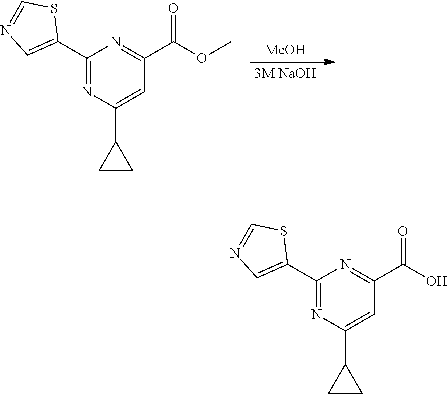 [SMILES: "MeOH" ; COC(=O)c1cc(C2CC2)nc(-c2cncs2)n1 ; "3M NaOH" ; O=C(O)c1cc(C2CC2)nc(-c2cncs2)n1]

Step 3: Preparation of 6-cyclopropyl-2-(thiazol-5-yl)pyrimidine-4-carboxylic acid. Methyl 6-cyclopropyl-2-(thiazol-5-yl)pyrimidine-4-carboxylate (255 mg, 0.976 mmol) was dissolved in MeOH (3 mL), 3 M aq. NaOH (976 mL, 2.93 mmol) was added and the mixture was stirred at r.t. for 30 min Most of the MeOH was evaporated under reduced pressure and the remaining aqueous phase pH was adjusted to ˜3 using 3 M aq. HCl). The resulting suspension was filtered providing 6-cyclopropyl-2-(thiazol-5-yl)pyrimidine-4-carboxylic acid (212 mg, 0.857 mmol, 88%) as a tan solid. LRMS (APCI) m/z 248.1 (M+H).

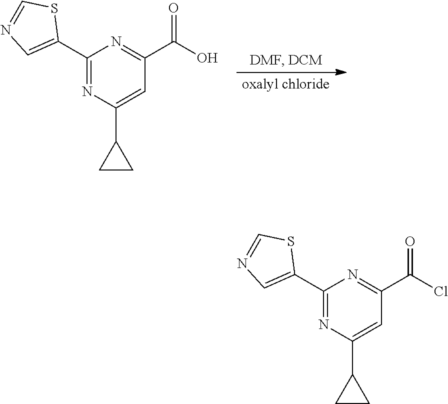

Step 4: Preparation of 6-cyclopropyl-2-(thiazol-5-yl)pyrimidine-4-carbonyl chloride. Prepared using same acyl chloride synthetic procedure as Compound 13.

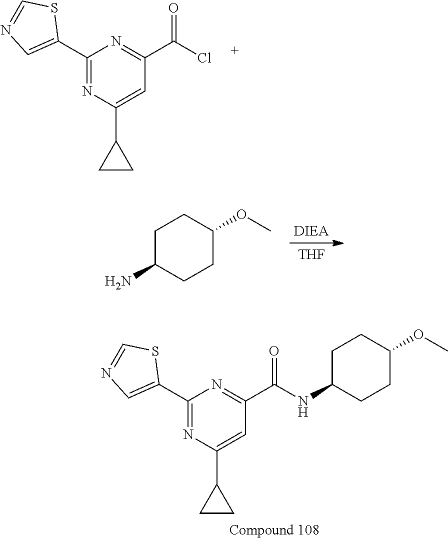

Step 5: Preparation of 6-cyclopropyl-N-((1r,4r)-4-methoxycyclohexyl)-2-(thiazol-5-yl)pyrimidine-4-carboxamide. Prepared using same amide bond formation procedure as Compound 13 to give 6-cyclopropyl-N-((1r,4r)-4-methoxycyclohexyl)-2-(thiazol-5-yl)pyrimidine-4-carboxamide (17 mg, 0.047 mmol, 42%) as a white solid. LRMS (APCI) m/z 359.1 (M+H). ¹H NMR (400 MHz, Methanol-d₄) δ 9.11 (s, 1H), 8.84 (s, 1H), 7.79 (s, 1H), 3.97-3.86 (m, 1H), 3.37 (s, 3H), 3.29-3.22 (m, 1H), 2.29-2.20 (m, 1H), 2.19-2.10 (m, 2H), 2.08-1.98 (m, 2H), 1.58 (qd, J=13.0, 3.3 Hz, 2H), 1.36 (tdd, J=13.1, 10.6, 3.5 Hz, 2H), 1.28-1.18 (m, 4H).

Compounds 109-124 were prepared using the methods provided in the table below.

| Compound | Method of Preparation |
|---|---|
| 109 | Prepared in same fashion as Compound 108 |
| 110 | Prepared in same fashion as Compound 108 |
| 111 | Beginning with 2-chloro-6- |
| | (trifluoromethyl)pyrimidine-4-carboxylic acid, |
| | amide bond formation as for Compound 13 followed |
| | by nucleophilic aromatic substitution as for |
| | Compound 36 |
| 112 | Beginning with methyl 2-bromopyrimidine-4- |
| | carboxylate, Stille coupling and ester hydrolysis |
| | as for Compound 11 followed by amide bond |
| | formation as for Compound 13 |
| 113 | Prepared in same fashion as Compound 112 |
| 114 | Prepared in same fashion as Compound 112 |
| 115 | Beginning with 2-chloro-6- |
| | (trifluoromethyl)pyrimidine-4-carboxylic acid, |
| | amide bond formation as for Compound 13 followed |
| | by Stille coupling as for Compound 11 |
| 116 | Beginning with 2-chloro-6- |
| | (trifluoromethyl)pyrimidine-4-carboxylic acid, |
| | amide bond formation as for Compound 13 followed |
| | by Stille coupling as for Compound 11 |
| 117 | Beginning with 2-(1H-imidazol-1-yl)-6- |
| | methylpyrimidine-4-carboxylic acid, amide bond |
| | formation as for Compound 163 |
| 118 | Beginning with 2-(1H-imidazol-1-yl)-6- |
| | methylpyrimidine-4-carboxylic acid, amide bond |
| | formation as for Compound 163 |
| 119 | Beginning with 2-(1H-imidazol-1-yl)-6- |
| | methylpyrimidine-4-carboxylic acid, amide bond |
| | formation as for Compound 163 |
| 120 | Beginning with 2-(1H-imidazol-1-yl)-6- |
| | methylpyrimidine-4-carboxylic acid, amide bond |
| | formation as for Compound 163 |
| 121 | Beginning with methyl 2-chloro-6- |
| | methylpyrimidine-4-carboxylate, Suzuki coupling |
| | as for Compound 59 followed by amide bond |
| | formation as for Compound 163 |
| 122 | Beginning with methyl 2-chloro-6- |
| | methylpyrimidine-4-carboxylate, Suzuki coupling |
| | as for Compound 59 followed by amide bond |
| | formation as for Compound 163 |
| 123 | Beginning with methyl 2-chloro-6- |
| | methylpyrimidine-4-carboxylate, Suzuki coupling |
| | as for Compound 59 followed by amide bond |
| | formation as for Compound 163 |
| 124 | Beginning with methyl 2-chloro-6- |
| | methylpyrimidine-4-carboxylate, Suzuki coupling |
| | as for Compound 59 followed by amide bond |
| | formation as for Compound 163 |

### Example 0

### Synthesis of Compounds 125 and 126

### Preparation of 2-(1H-imidazol-1-yl)-6-methoxy-N-((1r,4r)-4-methoxycyclohexyl)pyrimidine-4-carboxamide (Compound 125) and 6-hydroxy-2-(1H-imidazol-1-yl)-N-((1r,4r)-4-methoxycyclohexyl)pyrimidine-4-carboxamide (Compound 126)

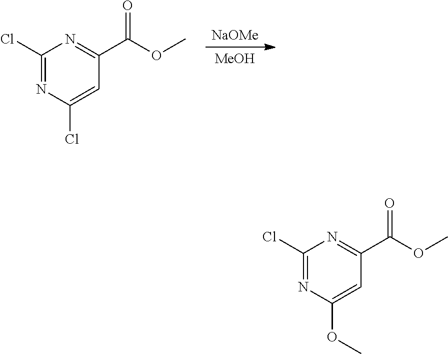

Step 1: Preparation of methyl 2-chloro-6-methoxypyrimidine-4-carboxylate. Methyl 2,6-dichloropyrimidine-4-carboxylate (1.08 g, 5.22 mmol) was dissolved in MeOH (25 mL) and cooled to 0° C. with an ice bath. NaOMe (1.13 g of 25 w/w % in MeOH, 5.22 mmol) and the mixture was stirred at 0° C. for 15 min followed by dilution with ethyl acetate (70 mL) and water (25 mL). The layers were separated and the organic phase was washed with brine, dried over sodium sulfate and concentrated under reduced pressure to provide methyl 2-chloro methoxypyrimidine-4-carboxylate (812 mg, 1.06 mmol, 77%) as a white solid which was used in the subsequent step without additional purification. LRMS (APCI) m/z 203.0 (M+H).

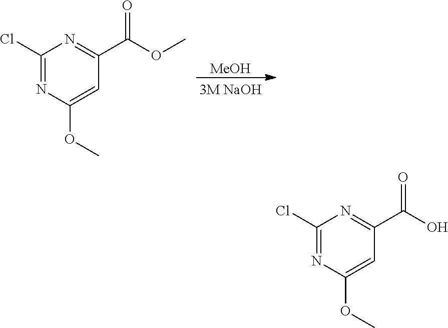

Step 2: Preparation of 2-chloro-6-methoxypyrimidine-4-carboxylic acid. Methyl 2-chloro-6-methoxypyrimidine-4-carboxylate (782 mg, 3.86 mmol) was dissolved in MeOH (10 mL) and cooled to 0° C. with an ice bath. 3 M aq. NaOH (1.41 mL, 3.86 mmol) was added and the mixture was stirred at 0° C. for 30 min. The pH of the reaction was adjusted to 4 using 3 M aq. HCl and then ethyl acetate (60 mL) was added followed by water (20 mL). The layers were shaken and separated and the organic phase was washed with brine, dried over sodium sulfate and concentrated under reduced pressure to give 2-chloro-6-methoxypyrimidine-4-carboxylic acid (727 mg, 3.85 mmol, 99%) as a white solid. LRMS (APCI) m/z 189.0 (M+H).

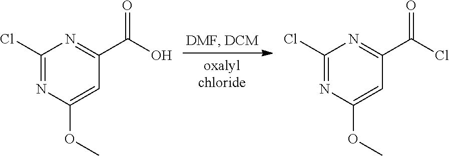

Step 3: Preparation of 2-chloro-6-methoxypyrimidine-4-carbonyl chloride. Prepared using same acyl chloride synthetic procedure as Compound 13.

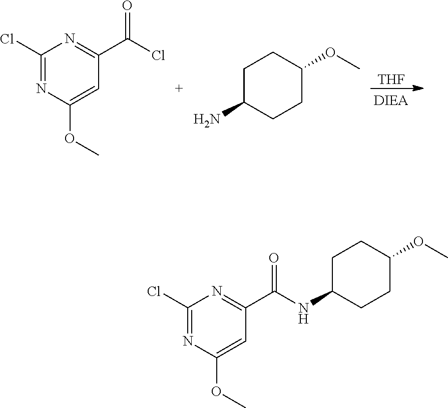

Step 4: Preparation of 2-Chloro-6-methoxy-N-((1r,4r)-4-methoxycyclohexyl)pyrimidine-4-carboxamide. Prepared using same amide bond formation procedure as Compound 13.

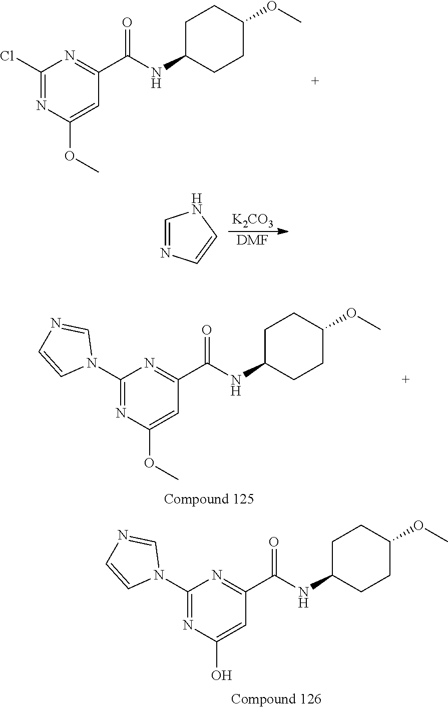

Step 5: Preparation of 2-(1H-imidazol-1-yl)-6-methoxy-N-((1r,4r)-4-methoxycyclohexyl)pyrimidine-4-carboxamide and 6-hydroxy-2-(1H-imidazol-1-yl)-N-((1r,4r)-4-methoxycyclohexyl)pyrimidine-4-carboxamide. 2-chloro-6-methoxy-N-((1r,4r)-4-methoxycyclohexyl)pyrimidine-4-carboxamide (61 mg, 0.204 mmol) was combined with 1H-imidazole (28 mg, 0.407 mmol) and K₂CO₃ (85 mg, 0.611 mmol). DMF (1 mL) was added and the mixture was heated at 100° C. for 1 h in an oil bath. The reaction was cooled to r.t. and purified using reverse phase HPLC with a 40 minute gradient from 5-100% ACN/water (Phenomenex Gemini 5 micron C18 Axia pack 150×21.2 mm column) to give 2-(1H-imidazol-1-yl)-6-methoxy-N-((1r,4r)-4-methoxycyclohexyl)pyrimidine-4-carboxamide (20 mg, 0.060 mmol, 30%) and hydroxy-2-(1H-imidazol-1-yl)-N-((1r,4r)-4-methoxycyclohexyl)pyrimidine-4-carboxamide (14 mg, 0.044 mmol, 22%) as white solids. Analytical data for 2-(1H-imidazol-1-yl)-6-methoxy-N-((1r,4r)-4-methoxycyclohexyl)pyrimidine-4-carboxamide: ¹H NMR (400 MHz, Methanol-d₄) δ 8.90 (s, 1H), 8.18 (t, J=1.3 Hz, 1H), 7.33 (s, 1H), 7.15 (t, J=1.2 Hz, 1H), 4.14 (s, 3H), 3.97-3.84 (m, 1H), 3.40-3.34 (m, 3H), 3.28-3.17 (m, 1H), 2.21-2.10 (m, 2H), 2.05-1.94 (m, 2H), 1.65-1.52 (m, 2H), 1.41-1.26 (m, 2H). LRMS (APCI) m/z 332.1 (M+H). Analytical data for 6-hydroxy-2-(1H-imidazol-1-yl)-N-((1r,4r)-4-methoxycyclohexyl)pyrimidine-4-carboxamide: ¹H NMR (400 MHz, Methanol-d₄) δ 8.74 (s, 1H), 8.06 (s, 1H), 7.05 (s, 1H), 6.82 (s, 1H), 3.92-3.82 (m, 1H), 3.38 (s, 3H), 3.3-3.22 (m, 1H), 2.20-2.10 (m, 2H), 2.07-1.97 (m, 2H), 1.64-1.50 (m, 2H), 1.41-1.28 (m, 2H). LRMS (APCI) m/z 318.1 (M+H).

Compounds 127-131 were prepared using the methods provided in the table below.

| Compound | Method of Preparation |
|---|---|
| 127 | Prepared in same fashion as Compound 125 |
| 128 | Prepared in same fashion as for Compound 125 with |
| | last step Suzuki coupling as for Compound 59 |
| 129 | Prepared in same fashion as for Compound 125 with |
| | last step Suzuki coupling as for Compound 59 |
| 130 | Prepared in same fashion as for Compound 125 with |
| | last step Stille coupling as for Compound 11 |
| 131 | Prepared in same fashion as Compound 125 with |
| | last step Stille coupling as for Compound 11 |

### Example P

### Synthesis of Compound 137

### Preparation of 2-(1H-imidazol-1-yl)-N-(6-methylpyridin-3-yl)-6-(trifluoromethyl)pyrimidine-4-carboxamide (Compound 137)

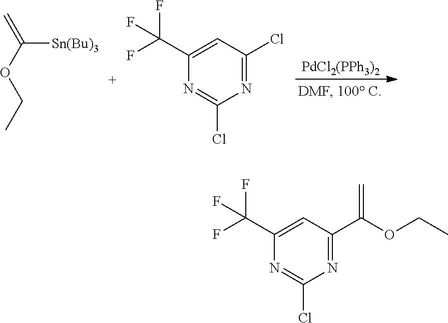

Step 1: Preparation of 2-chloro-4-(1-ethoxyvinyl)-6-(trifluoromethyl)pyrimidine. To a stirred solution of 2,4-dichloro-6-(trifluoromethyl)pyrimidine (2.0 g, 9.2 mmol) in DMF (20 mL) at r.t. were added tributyl(1-ethoxyvinyl)stannane (3.35 g, 9.28 mmol, 1.01) and trans-dichlorobis(triphenylphosphine)palladium(II) (1.3 g, 1.85 mmol). The resulting mixture was stirred at 100° C. for 18 h under a nitrogen atmosphere, cooled to r.t., diluted with water (50 mL) and extracted with ethyl acetate (2×30 mL). The combined organic layers were washed with brine, dried over anhydrous Na₂SO₄, concentrated under reduced pressure and purified by silica gel column chromatography using petroleum ether/ethyl acetate (50:1) to afford 2-chloro-4-(1-ethoxyvinyl)-6-(trifluoromethyl)pyrimidine (2.0 g, 7.9 mmol, 87%) as a yellow oil. ¹H NMR (300 MHz, DMSO-d₆) δ 7.96 (s, 1H), 5.71 (d, J=2.7 Hz, 1H), 4.90 (d, J=2.7 Hz, 1H), 4.02 (q, J=7.0 Hz, 2H), 0.88 (td, J=7.3, 2.0 Hz, 3H).

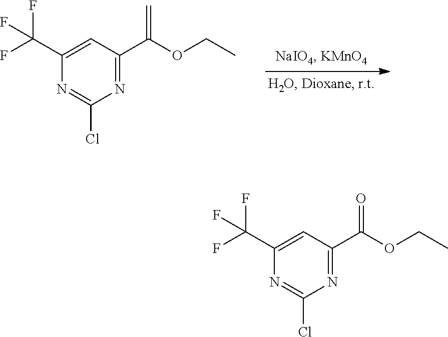 [SMILES: C=C(OCC)c1cc(C(F)(F)F)nc(Cl)n1 ; CCOC(=O)c1cc(C(F)(F)F)nc(Cl)n1]

Step 2: Preparation of ethyl 2-chloro-6-(trifluoromethyl)pyrimidine-4-carboxylate. To a stirred solution of 2-chloro-4-(1-ethoxyvinyl)-6-(trifluoromethyl)pyrimidine (1.5 g, 5.95 mmol) in dioxane (15 mL) at r.t. were added NaIO₄ (510 mg, 2.38 mmol) in H₂O (3 mL) and KMnO₄ (1.88 g, 11.89 mmol). The resulting mixture was stirred at r.t. for 2 h, filtered and the filter cake was washed three times with MeOH (10 mL). The filtrate was concentrated under reduced pressure and purified with silica gel column chromatography using petroleum ether/ethyl acetate (50:1) to afford ethyl 2-chloro-6-(trifluoromethyl)pyrimidine carboxylate (200 mg, 13%) as a white solid. ¹H NMR (400 MHz, DMSO-d₆) δ 8.42 (s, 1H), 4.45 (q, J=7.1 Hz, 2H), 1.37 (t, J=7.1 Hz, 3H).

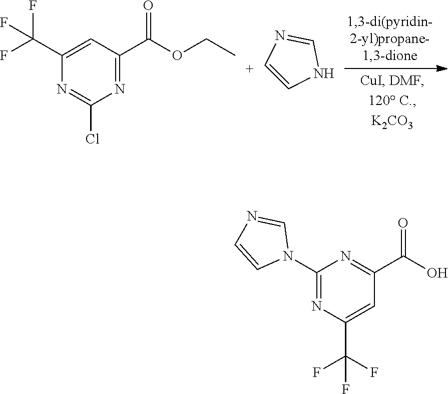 [SMILES: CCOC(=O)c1cc(C(F)(F)F)nc(Cl)n1 ; [C+]1=CNC=N1 ; O=C(O)c1cc(C(F)(F)F)nc(-n2ccnc2)n1]

Step 3: Preparation of 2-(1H-imidazol-1-yl)-6-(trifluoromethyl)pyrimidine-4-carboxylic acid. To a stirred solution of ethyl 2-chloro-6-(trifluoromethyl)pyrimidine-4-carboxylate (200 mg, 0.786 mmol) in DMF (4 mL) at r.t. were added imidazole (64 mg, 0.940 mmol), K₂CO₃ (216 mg, 1.563 mmol), CuI (15 mg, 0.079 mmol) and 1,3-bis(pyridin-2-yl)propane-1,3-dione (18 mg, 0.080). The resulting mixture was stirred at 120° C. for 18 under a nitrogen atmosphere, cooled to r.t. and purified by C18 column Chromatography using water (0.05% NH₄HCO₃): ACN=20:1) as the mobile phase to afford 2-(1H-imidazol-1-yl)-6-(trifluoromethyl)pyrimidine-4-carboxylic acid (120 mg, 0.47 mmol, 59%) as white solid. LRMS (ES) m/z 259 (M+H).

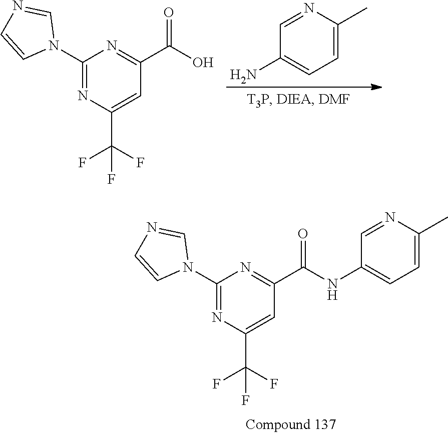

Step 4: Preparation of 2-(1H-imidazol-1-yl)-N-(6-methylpyridin-3-yl)-6-(trifluoromethyl)pyrimidine-4-carboxamide. Prepared using same amide bond formation procedure as Compound 165. LRMS (ES) m/z 349 (M+H).: ¹H NMR (300 MHz, DMSO-d₆) δ 10.96 (s, 1H), 9.10 (t, J=1.1 Hz, 1H), 8.90 (d, J=2.5 Hz, 1H), 8.36-8.27 (m, 2H), 8.15 (dd, J=8.4, 2.6 Hz, 1H), 7.37 (d, J=8.4 Hz, 1H), 7.27 (t, J=1.3 Hz, 1H), 2.52 (s, 3H).

Compounds 138-142 and 144-157 were prepared using the methods provided in the table below.

| Compound Number | Method of Preparation |
|---|---|
| 138 | Prepared in same fashion as Compound 137 |
| 139 | Beginning with methyl 2-bromopyrimidine-4- |
| | carboxylate, prepared in same fashion as |
| | Compound 11 |
| 140 | Beginning with methyl 2-bromopyrimidine-4- |
| | carboxylate, prepared in same fashion as |
| | Compound 11 |
| 141 | Prepared in same fashion as Compound 108 |
| 142 | Prepared in same fashion as Compound 108 |
| 144 | Prepared in same fashion as Compound 109 |
| 145 | Prepared in same fashion as Compound 109 |
| 146 | Prepared in same fashion as Compound 109 |
| 147 | Beginning with 4-chloropyrimidine-2-carboxylic |
| | acid, amide bond formation as for Compound 13 |
| | followed by Suzuki coupling as for Compound 59 |
| 148 | Beginning with 4-chloropyrimidine-2-carboxylic |
| | acid, amide bond formation as for Compound 13 |
| | followed by Suzuki coupling as for Compound 59 |
| 149 | Beginning with 4-chloropyrimidine-2-carboxylic |
| | acid, amide bond formation as for Compound 13 |
| | followed by Suzuki coupling as for Compound 59 |
| 150 | Beginning with 4-chloropyrimidine-2-carboxylic |
| | acid, amide bond formation as for Compound 13 |
| | followed by Stille coupling as for Compound 11 |
| 151 | Prepared in same fashion as Compound 137 except |
| | second step Suzuki coupling as for Compound 59 |
| 152 | Prepared in same fashion as Compound 137 except |
| | second step Suzuki coupling as for Compound 59 |
| 153 | Prepared in same fashion as Compound 137 except |
| | second step Suzuki coupling as for Compound 59 |
| 154 | Prepared in same fashion as Compound 137 except |
| | second step Suzuki coupling as for Compound 59 |
| 155 | Beginning with methyl 2-chloro-6- |
| | methylpyrimidine-4-carboxylate, Suzuki coupling |
| | as for Compound 59 followed by amide bond |
| | formation as for Compound 163 |
| 156 | Beginning with 2-(1H-imidazol-1-yl)-6- |
| | methylpyrimidine-4-carboxylic acid, amide bond |
| | formation as for Compound 163 |
| 157 | Prepared in same fashion as Compound 137 |

### Example Q

### Synthesis of Compound 178

### Preparation of 6-(2-Hydroxypropan-2-yl)-N-((1r,4r)-4-methoxycyclohexyl)-2-(1-methyl-1H-imidazol-5-yl)pyrimidine-4-carboxamide (Compound 178)

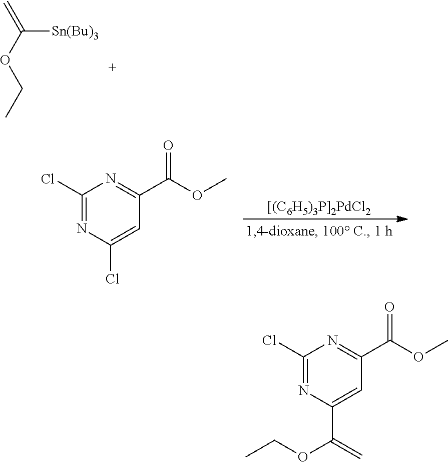

Step 1: Preparation of methyl 2-chloro-6-(1-ethoxyvinyl)pyrimidine-4-carboxylate. Methyl 2,6-dichloropyrimidine-4-carboxylate (5.0 g, 24.15 mmol) was combined with tributyl(1-ethoxyvinyl)stannane (8.16 mL, 24.15 mmol) and trans-dichlorobis(triphenylphosphine)palladium(II) (848 mg, 1.21 mmol). 1,4-dioxane was added (25 mL) and the mixture was heated in an oil bath under an atmosphere of nitrogen at 100° C. for 1 h, followed by 50° C. for 18 h. The mixture was cooled to r.t., the solvent was evaporated in vacuo and the product was purified with silica gel using 15% ethyl acetate/hexanes to provide methyl 2-chloro-6-(1-ethoxyvinyl)pyrimidine-4-carboxylate (4.10 g, 16.9 mmol, 70%) as a white solid. LRMS (APCI) m/z 243.0 (M+H).

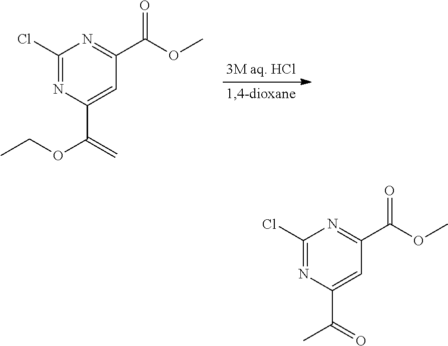 [SMILES: C=C(OCC)c1cc(C(=O)OC)nc(Cl)n1 ; COC(=O)c1cc(C(C)=O)nc(Cl)n1]

Step 2: Preparation of methyl 6-acetyl-2-chloropyrimidine-4-carboxylate. Methyl 2-chloro-6-(1-ethoxyvinyl)pyrimidine-4-carboxylate (1.45 g, 5.96 mmol) was dissolved in 1,4-dioxane (25 mL) and 3 M aq. HCl (1.99 ml, 5.96 mmol) was added. The resulting solution was heated in an oil bath at 50° C. for 3 h. Upon cooling to r.t., the reaction was carefully neutralized with saturated aqueous NaHCO₃. The resulting mixture was extracted with ethyl acetate (2×75 mL), the organic extracts were combined, washed with brine, dried over sodium sulfate and concentrated under reduced pressure to provide methyl 6-acetyl-2-chloropyrimidine carboxylate (1.08 g, 5.05 mmol, 85%) as a tan solid which was used in the next step without additional purification. LRMS (APCI) m/z 215.1 (M+H).

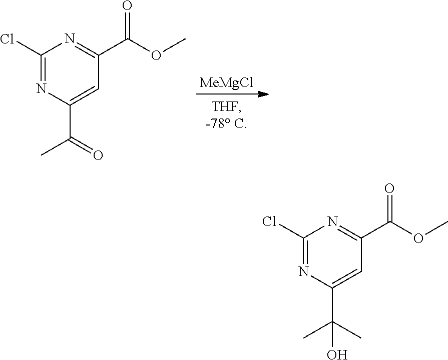

Step 3: Preparation of methyl 2-chloro-6-(2-hydroxypropan-2-yl)pyrimidine-4-carboxylate. Methyl 6-acetyl-2-chloropyrimidine-4-carboxylate (1.07 g, 4.97 mmol) was dissolved in anhydrous THF (10 mL) under a nitrogen atmosphere and cooled to −78° C. using an acetone/dry-ice bath. MeMgCl (1.66 ml of 3.0 M solution in THF, 4.97 mmol) was added dropwise with a syringe and the resulting mixture was stirred at −78° C. for 15 min. The reaction was quenched with saturated aqueous NH₄Cl (1 mL) and diluted with water (10 mL) and ethyl acetate (40 mL). The layers were shaken and separated, the organic phase was washed with brine, dried over sodium sulfate, concentrated under reduced pressure and purified with silica gel using 30% ethyl acetate/hexanes to give methyl 2-chloro-6-(2-hydroxypropan-2-yl)pyrimidine-4-carboxylate (290 mg, 1.15 mmol, 25%) as a white solid. LRMS (APCI) m/z 231.0 (M+H). ¹H NMR (400 MHz, Methanol-d₄) δ 8.27 (s, 1H), 4.01 (s, 3H), 1.54 (s, 6H).

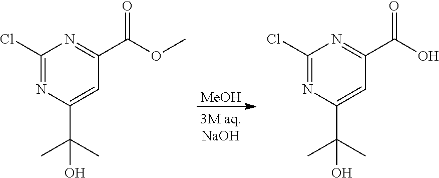

Step 4: Preparation of 2-chloro-6-(2-hydroxypropan-2-yl)pyrimidine-4-carboxylic acid. Prepared using same ester hydrolysis procedure as Compound 11.

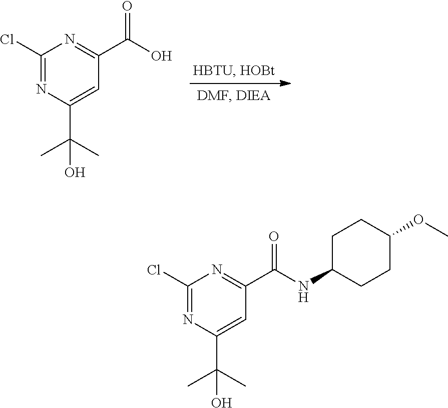 [SMILES: CC(C)(O)c1cc(C(=O)O)nc(Cl)n1 ; CO[C@H]1CC[C@H](NC(=O)c2cc(C(C)(C)O)nc(Cl)n2)CC1]

Step 5: Preparation of 2-Chloro-6-(2-hydroxypropan-2-yl)-N-((1r,4r)-4-methoxycyclohexyl)pyrimidine-4-carboxamide. Prepared using same amide bond formation procedure as Compound 12.

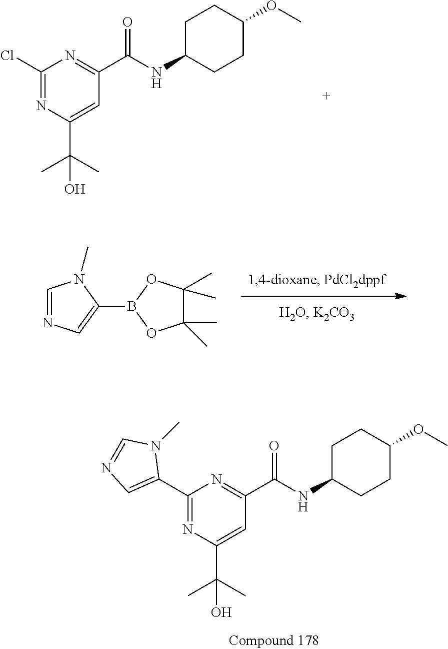 [SMILES: CO[C@H]1CC[C@H](NC(=O)c2cc(C(C)(C)O)nc(Cl)n2)CC1 ; Cn1cncc1B1OC(C)(C)C(C)(C)O1 ; CO[C@H]1CC[C@H](NC(=O)c2cc(C(C)(C)O)nc(-c3cncn3C)n2)CC1]

Step 6: Preparation of 6-(2-Hydroxypropan-2-yl)-N-((1r,4r)-4-methoxycyclohexyl)-2-(1-methyl-1H-imidazol-5-yl)pyrimidine-4-carboxamide. Prepared using same Suzuki coupling procedure as Compound 59 to give 6-(2-hydroxypropan-2-yl)-N-((1r,4r)-4-methoxycyclohexyl)-2-(1-methyl-1H-imidazol-5-yl)pyrimidine-4-carboxamide (25 mg, 0.067 mmol, 48%) as a white solid. ¹H NMR (400 MHz, Methanol-d₄) δ 8.15 (s, 1H), 8.10 (s, 1H), 7.86 (s, 1H), 4.16 (s, 3H), 3.97-3.82 (m, 1H), 3.36 (s, 3H), 3.28-3.21 (m, 1H), 2.18-2.09 (m, 2H), 2.08-1.98 (m, 3H), 1.65-1.49 (m, 8H), 1.42-1.28 (m, 2H). LRMS (APCI) m/z 374.2 (M+H).

Compounds 158-162, 164, 165, and 167-177 were prepared using the methods provided in the table below.

| Compound Number | Method of Preparation |
|---|---|
| 158 | Prepared in same fashion as Compound 178 with |
| | last step nucleophilic aromatic substitution |
| | procedure as for Compound 36 |
| 159 | Prepared in same fashion as Compound 178 |
| 160 | Prepared in same fashion as Compound 108 with |
| | second step nucleophilic aromatic substitution |
| | procedure as for Compound 36 |
| 161 | Prepared in same fashion as Compound 108 with |
| | second step nucleophilic aromatic substitution |
| | procedure as for Compound 36 |
| 162 | Prepared in same fashion as Compound 108 with |
| | second step nucleophilic aromatic substitution |
| | procedure as for Compound 36 |
| 164 | Prepared in same fashion as Compound 137 |
| 165 | Beginning with methyl 2-chloro-6- |
| | trifluoromethylpyrimidine-4-carboxylate, Suzuki |
| | coupling as for Compound 59 followed by amide |
| | bond formation as for Compound 163 |
| 167 | Beginning with ethyl 2-chloro-6- |
| | isopropylpyrimidine-4-carboxylate, ester |
| | hydrolysis as for Compound 108 and amide bond |
| | formation as for Compound 13 followed by |
| | nucleophilic aromatic substitution as for |
| | Compound 36 |
| 168 | Prepared in same fashion as for Compound 167 |
| 169 | Prepared in same fashion as for Compound 167 |
| 170 | Prepared in same fashion as for Compound 167 |
| 171 | Beginning with ethyl 2-chloro-6- |
| | isopropylpyrimidine-4-carboxylate, ester |
| | hydrolysis as for Compound 108 and amide bond |
| | formation as for Compound 13 followed by Suzuki |
| | coupling as for Compound 59 |
| 172 | Prepared in same fashion as Compound 171 |
| 173 | Prepared in same fashion as Compound 171 |
| 174 | Prepared in same fashion as Compound 171 |
| 175 | Prepared in same fashion as Compound 178 with |
| | last step nucleophilic aromatic substitution |
| | procedure as for Compound 36 |
| 176 | Prepared in same fashion as Compound 178 |
| 177 | Prepared in same fashion as Compound 178 |

### Example R

### Synthesis of Compound 181

### Preparation of 6-Cyclobutyl-N-((1r,4r)-4-methoxycyclohexyl)-2-(1-methyl-1H-imidazol-5-yl)pyrimidine-4-carboxamide (Compound 181)

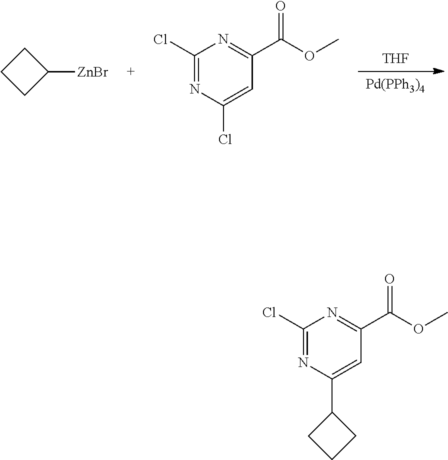

Step 1: Preparation of methyl 2-chloro-6-cyclobutylpyrimidine-4-carboxylate. To an oven dried 250 mL round bottom flask was added methyl 2,6-dichloropyrimidine-4-carboxylate (2.0 g, 9.66 mmol) followed by tetrakis(triphenylphosphine)palladium(0) (558 mg, 0.483 mmol). The reaction flask was evacuated and backfilled with nitrogen 3 times and anhydrous THF (12 mL) was added using a syringe, followed by cyclobutylzinc(II) bromide (21.26 mL of 0.5 M in THF, 10.63 mmol). The resulting mixture was stirred at 50° C. for 2 h in an oil bath, cooled to r.t., concentrated under reduced pressure, combined with ethyl acetate (75 mL) and saturated aqueous NaHCO₃ (50 mL), stirred vigorously for 5 min and filtered through celite. The layers were separated and the organic phase was washed with brine, dried over sodium sulfate, concentrated in vacuo and purified with silica gel using 30% ethyl acetate/hexanes to give methyl 2-chloro-6-cyclobutylpyrimidine-4-carboxylate (1.20 g, 5.30 mmol, 55%) as a faintly yellow oil. ¹H NMR (400 MHz, DMSO-d₆) δ 7.89 (s, 21H), 3.92 (s, 3H), 3.86-3.72 (m, 1H), 2.38-2.22 (m, 4H), 2.12-1.96 (m, 1H), 1.94-1.74 (m, 1H). LRMS (APCI) m/z 227.1 (M+H).

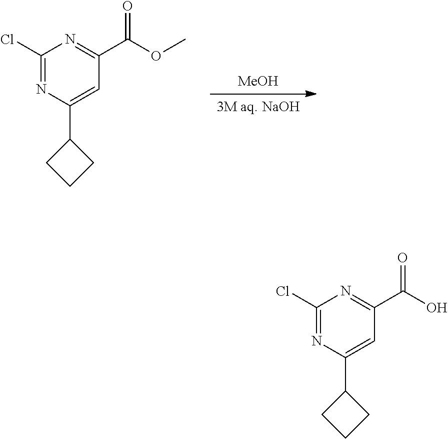

Step 2: Preparation of 2-chloro-6-cyclobutylpyrimidine-4-carboxylic acid. Prepared using same ester hydrolysis procedure as Compound 108.

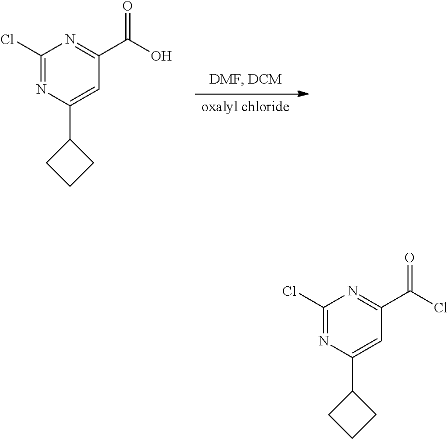

Step 3: Preparation of 2-chloro-6-cyclobutylpyrimidine-4-carbonyl chloride. Prepared using same acyl chloride procedure as Compound 13.

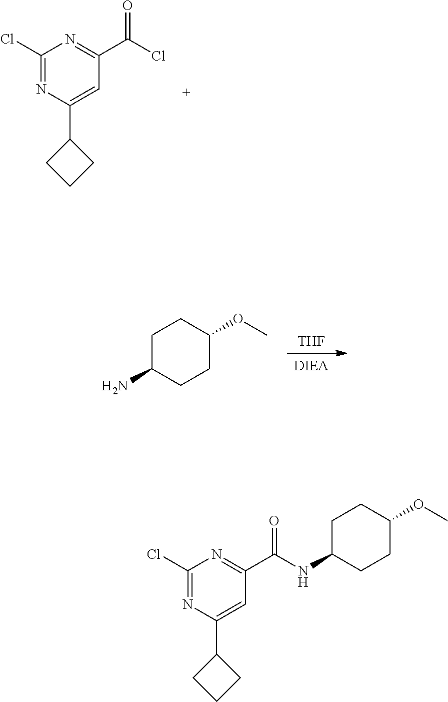

Step 4: Preparation of 2-Chloro-6-cyclobutyl-N-((1r,4r)-4-methoxycyclohexyl)pyrimidine-4-carboxamide. Prepared using same amide bond formation procedure as Compound 13.

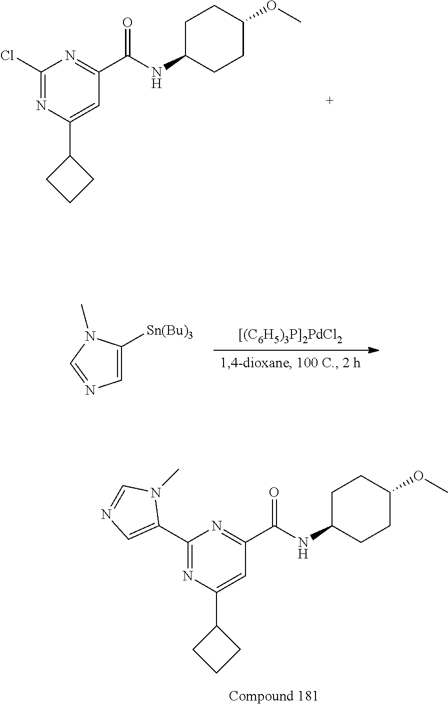

Step 5: Preparation of 6-Cyclobutyl-N-((1r,4r)-4-methoxycyclohexyl)-2-(1-methyl-1H-imidazol-5-yl)pyrimidine-4-carboxamide. 2-Chloro-6-cyclobutyl-N-((1r,4r)-4-methoxycyclohexyl)pyrimidine-4-carboxamide (100 mg, 0.295 mmol) was combined with 1-methyl-5-(tributylstannyl)-1H-imidazole (110 mg, 0.295 mmol), dichlorobis(triphenylphosphine)palladium(II) (21 mg, 0.021 mmol) and 1,4-dioxane (2 mL). The resulting mixture was heated in an oil bath in a sealed tube under a nitrogen atmosphere at 100° C. for 2 h, cooled to r.t., concentrated and purified using reverse phase HPLC with a 40 minute gradient from 5-100% ACN/water (Phenomenex Gemini 5 micron C18 Axia pack 150×21.2 mm column) to provide 6-cyclobutyl-N-((1r,4r)-4-methoxycyclohexyl)-2-(1-methyl-1H-imidazol-5-yl)pyrimidine-4-carboxamide (53 mg, 0.143 mmol, 31%) as a white solid. ¹H NMR (400 MHz, Methanol-d₄) δ 8.20 (s, 1H), 8.09 (s, 1H), 7.72 (s, 1H), 3.98-3.77 (m, 2H), 3.39 (s, 3H), 3.31-3.21 (m, 1H), 2.46 (td, J=8.6, 6.2 Hz, 4H), 2.23-2.11 (m, 3H), 2.09-1.95 (m, 3H), 1.64-1.50 (m, 2H), 1.43-1.29 (m, 12H). LRMS (APCI) m/z 370.2 (M+H).

Compounds 55, 56, 88-92, 102-105, and 182-186 were prepared using the methods provided in the table below.

| Compound Number | Method of Preparation |
|---|---|
| 55 | Beginning with 6-bromopyrazine-2-carboxylic acid, |
| | prepared in same fashion Compound 13 |
| 56 | Beginning with 6-bromopyrazine-2-carboxylic acid, |
| | prepared in same fashion Compound 13 |
| 88 | Beginning with 6-bromopyrazine-2-carboxylic acid, |
| | prepared in same fashion Compound 13 |
| 89 | Beginning with 6-bromopyrazine-2-carboxylic acid, |
| | prepared in same fashion Compound 13 |
| 90 | Beginning with 6-bromopyrazine-2-carboxylic acid, |
| | amide bond formation as Compound 13 followed by |
| | Suzuki coupling as Compound 59 |
| 91 | Beginning with 6-bromopyrazine-2-carboxylic acid, |
| | amide bond formation as Compound 13 followed by |
| | Suzuki coupling as Compound 59 |
| 92 | Beginning with 6-bromopyrazine-2-carboxylic acid, |
| | amide bond formation as Compound 13 followed by |
| | Suzuki coupling as Compound 59 |
| 102 | Beginning with 6-bromopyrazine-2-carboxylic acid, |
| | prepared in same fashion Compound 13 |
| 103 | Beginning with 6-bromopyrazine-2-carboxylic acid, |
| | prepared in same fashion Compound 13 |
| 104 | Beginning with 6-bromopyrazine-2-carboxylic acid, |
| | amide bond formation as Compound 13 followed by |
| | Suzuki coupling as Compound 59 |
| 105 | Beginning with 6-bromopyrazine-2-carboxylic acid, |
| | amide bond formation as Compound 13 followed by |
| | Suzuki coupling as Compound 59 |
| 182 | Prepared in same fashion as Compound 181 |
| 183 | Prepared in same fashion as Compound 181 |
| 184 | Prepared in same fashion as Compound 181 except |
| | last step nucleophilic aromatic substitution as |
| | Compound 36 |
| 185 | Prepared in same fashion as Compound 181 except |
| | last step nucleophilic aromatic substitution as |
| | Compound 36 |
| 186 | Prepared in same fashion as Compound 181 except |
| | last step nucleophilic aromatic substitution as |
| | Compound 36 |

### Example S

### Synthesis of Compound 145

### Preparation of 4-(1H-Imidazol-1-yl)-N-((1r,4r)-4-methoxycyclohexyl)pyrimidine-2-carboxamide

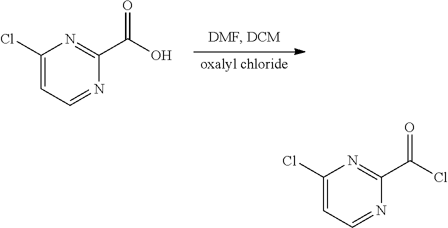

Step 1: Preparation of 4-Chloropyrimidine-2-carbonyl chloride. Prepared using same procedure as Compound 13 and used in the subsequent step without additional purification to give 4-chloropyrimidine-2-carbonyl chloride (558 mg, 3.15 mmol, quantitative yield) as a glassy solid.

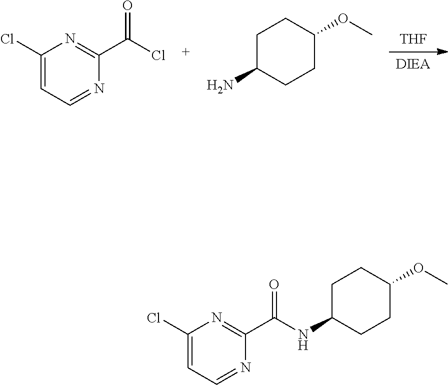

Step 2: Preparation of 4-Chloro-N-((1r,4r)-4-methoxycyclohexyl)pyrimidine-2-carboxamide. Prepared using the same procedure as Compound 13 and purified with silica gel using 10% MeOH/DCM to afford 4-chloro-N-((1r,4r)-4-methoxycyclohexyl)pyrimidine-2-carboxamide (847 mg, 3.14 mmol) as a sticky yellow solid. LRMS (ES) m/z 270.0 (M+H).

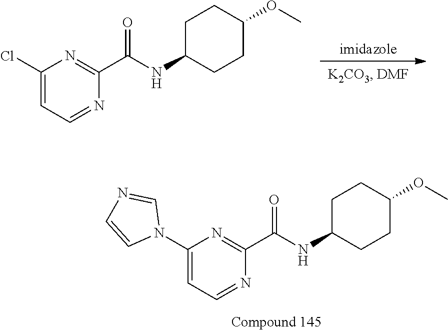

Step 3: Preparation of 4-(1H-Imidazol-1-yl)-N-((1r,4r)-4-methoxycyclohexyl)pyrimidine-2-carboxamide. Prepared using the same procedure as Compound 36 and purified using reverse phase HPLC with a 40 minute gradient from 0-100% ACN/water (Phenomenex Gemini 5 micron C18 Axia pack 150×21.2 mm column) to provide 4-(1H-imidazol-1-yl)-N-((1r,4r)-4-methoxycyclohexyl)pyrimidine-2-carboxamide (447 mg, 1.26 mmol, 85%) as a white solid. LRMS (ES) m/z 302.0 (M+H). ¹H NMR (400 MHz, DMSO-d₆) δ 9.04 (d, J=5.6 Hz, 1H), 8.92 (s, 1H), 8.71 (d, J=8.7 Hz, 1H), 8.23 (s, 1H), 8.06 (d, J=5.6 Hz, 1H), 7.23 (s, 1H), 3.88-3.74 (m, 1H), 3.26 (s, 3H), 3.19-3.07 (m, 1H), 2.05 (d, J=13.0 Hz, 2H), 1.86 (d, J=13.0 Hz, 2H), 1.61-1.43 (m, 2H), 1.31-1.16 (m, 2H).

### Example T

### Synthesis of Compound 148

### Preparation of N-((1r,4r)-4-methoxycyclohexyl)-4-(1-methyl-1H-imidazol-5-yl)pyrimidine-2-carboxamide

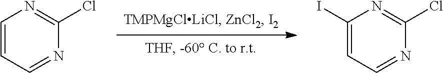 [SMILES: Clc1ncccn1 ; "THF, -60° C. to r.t." ; "TMPMgCl·LiCl, ZnCl2, I2" ; Clc1nccc(I)n1]

Step 1: Preparation of 2-Chloro-4-iodopyrimidine. To a stirred solution of 2-chloropyrimidine (20.0 g, 174.6 mmol) in THF (300 mL) at −60° C. was added 2,2,6,6-tetramethylpiperidinylmagnesium chloride lithium chloride complex solution (1.0 M in THF, 192.1 mL, 192.1 mmol) dropwise over 20 min under a nitrogen atmosphere. The resulting mixture was stirred at −60° C. for 2 h and then ZnCl₂ (0.7 M in THF, 274.4 mL, 192.1 mmol) was added at r.t. dropwise over 30 min, followed by stirring at r.t. for 1 h. Iodine (66.5 g, 261.9 mmol) in THF (100 ml) was added dropwise over 10 min and the resulting mixture was stirred at r.t. for 1 h, quenched with saturated aqueous NH₄Cl (300 mL), aqueous Na₂S₂O₃ (300 mL) and extracted twice with EtOAc (300 mL). The organic layers were combined, washed with brine (500 mL), dried over anhydrous Na₂SO₄, concentrated under reduced pressure and purified with silica gel column chromatography using 10% EtOAc/petroleum ether to afford 2-chloro-4-iodopyrimidine (25.0 g, 104.0 mmol, 60%) as a yellow solid. LRMS (ES) m/z 241 (M+H).

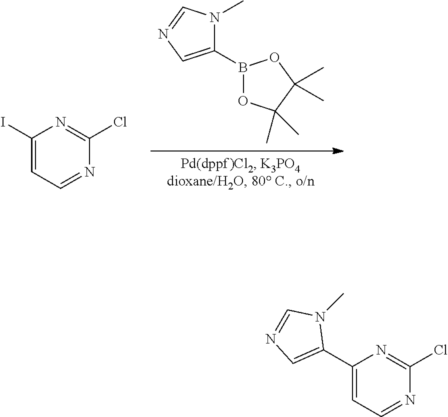

Step 2: Preparation of 2-Chloro-4-(1-methyl-1H-imidazol-5-yl)pyrimidine. To a stirred solution of 2-chloro-4-iodopyrimidine (24.2 g, 100.9 mmol, 1.1 equiv) and 1-methyl-5-(4,4,5,5-tetramethyl-1,3,2-dioxaborolan-2-yl)imidazole (19.1 g, 91.7 mmol, 1 equiv) in 1,4-dioxane (200 mL) and water (20 mL) were added Pd(dppf)Cl₂.CH₂Cl₂ (7.5 g, 9.2 mmol, 0.10 equiv) and K₃PO₄ (38.9 g, 183.4 mmol, 2.00 equiv). The resulting mixture was stirred at 80° C. for 18 h under a nitrogen atmosphere, cooled to r.t. and filtered. The filtrate was concentrated under reduced pressure, and the product was purified with silica gel using 10% MeOH/DCM to afford 2-chloro-4-(1-methyl-1H-imidazol-5-yl)pyrimidine (15.0 g, 77.1 mmol, 84%) as a brown oil. LRMS (ES) m/z 195 (M+H).

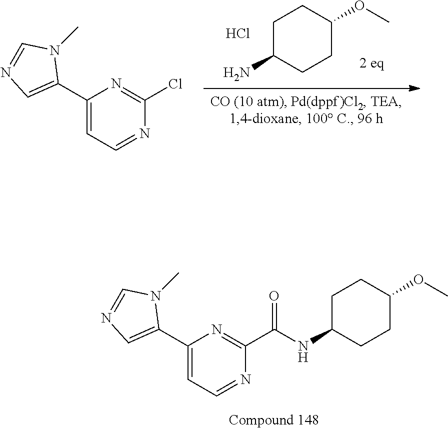

Step 3: Preparation of N-((1r,4r)-4-methoxycyclohexyl)-4-(1-methyl-1H-imidazol-5-yl)pyrimidine-2-carboxamide. To a solution of 2-chloro-4-(1-methyl-1H-imidazol-5-yl)pyrimidine (15.0 g, 77.1 mmol, 1 equiv) and (1r,4r)-4-methoxycyclohexan-1-amine hydrochloride (25.6 g, 154.2 mmol, 2.0 equiv) in dioxane (300 mL) were added Pd(dppf)Cl₂ (5.6 g, 7.7 mmol, 0.1 equiv) and TEA (23.4 g, 231.3 mmol, 3 equiv) in a pressure reactor. The resulting mixture was purged with nitrogen for 2 min and then pressurized to 10 atm with carbon monoxide and stirred at 100° C. for 48 h. Additional Pd(dppf)Cl₂ (5.6 g, 7.7 mmol, 0.1 equiv) and TEA (15.6 g, 154.2 mmol, 2 equiv) were added, the mixture was purged with nitrogen for 2 min, pressurized to 10 atm with carbon monoxide and stirred at 100° C. for 48 h. The reaction mixture was cooled to r.t., filtered, concentrated under reduced pressure and purified twice by C18 column chromatography, eluting with water (0.05% NH₄HCO₃)/MeCN (2:1) to afford N-((1r,4r)-4-methoxycyclohexyl)-4-(1-methyl-1H-imidazol yl)pyrimidine-2-carboxamide (9.7 g, 30.8 mmol, 40%) as an off-white solid. LRMS (ES) m/z 316 [M+H]. ¹H NMR (300 MHz, DMSO-d₆) δ 8.83 (d, J=5.4 Hz, 1H), 8.47 (d, J=8.2 Hz, 1H), 7.99-7.90 (m, 3H), 4.07 (s, 3H), 3.85-3.62 (m, 1H), 3.24 (s, 3H), 3.11 (td, J=10.3, 5.1 Hz, 1H), 2.02 (d, J=12.3 Hz, 2H), 1.87 (d, J=12.5 Hz, 2H), 1.55-1.36 (m, 2H), 1.32-1.14 (m, 2H).

### Example U

### Synthesis of Compound 189

### Preparation of 4-(tert-butyl)-N-(6-(difluoromethyl)pyridin-3-yl)-6-(1-methyl-1H-imidazol-5-yl)pyrimidine-2-carboxamide

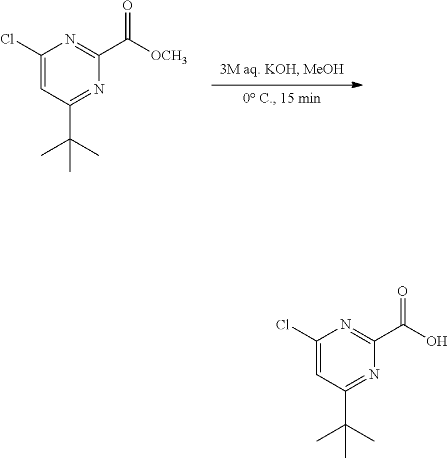

Step 1: Preparation of 4-(tert-Butyl)-6-chloropyrimidine-2-carboxylic acid. Methyl 4-(tert-Butyl)-6-chloropyrimidine-2-carboxylate (661 mg, 2.89 mmol) was dissolved in MeOH (5 mL) and cooled to 0° C. with an ice bath. 3 M aq. KOH (1.06 mL, 3.18 mmol) was added and the resulting mixture was stirred at 0° C. for 15 min. The pH was adjusted to 3-4 using 3 M aq. HCl and the resulting homogeneous solution was extracted with EtOAc (2×30 mL). The organic extracts were combined, dried over sodium sulfate and concentrated under reduced pressure to provide 4-(tert-butyl)-6-chloropyrimidine-2-carboxylic acid (522 mg, 2.43 mmol, 84% yield) as a white solid. LRMS (APCI) m/z 215.0 (M+H).

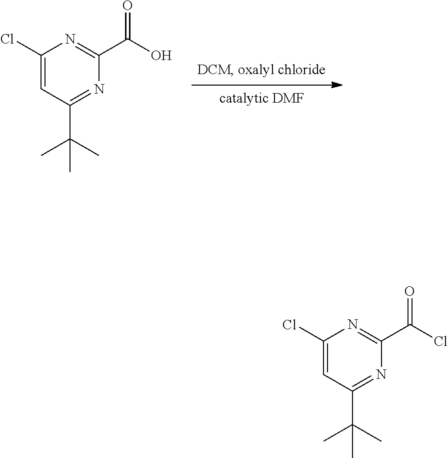

Step 2: Preparation of 4-(tert-Butyl)-6-chloropyrimidine-2-carbonyl chloride. 4-(tert-Butyl)-6-chloropyrimidine-2-carboxylic acid (522 mg, 2.43 mmol) was suspended in DCM (5 mL) and oxalyl chloride (1.46 mL of 2.0 M in DCM, 2.92 mmol) was added, followed by DMF (18 mg, 0.24 mmol). The resulting mixture was stirred at r.t. for 30 min. The solvent was evaporated under reduced pressure to provide 4-(tert-butyl)-6-chloropyrimidine-2-carbonyl chloride (0.566 mg, 2.43 mmol) as a glassy solid.

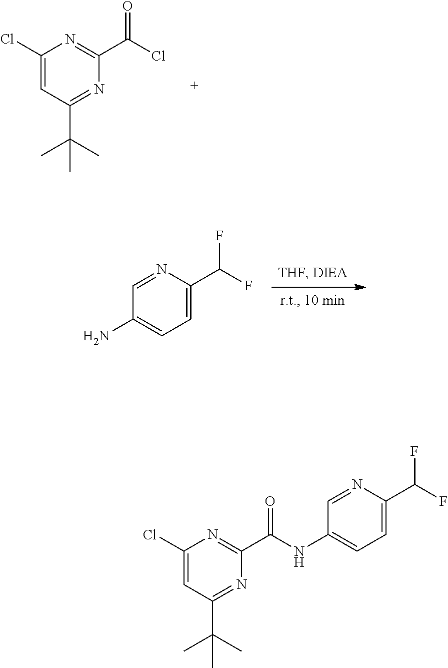 [SMILES: CC(C)(C)c1cc(Cl)nc(C(=O)Cl)n1 ; Nc1ccc(C(F)F)nc1 ; "THF, DIEA" ; "r.t., 10 min" ; CC(C)(C)c1cc(Cl)nc(C(=O)Nc2ccc(C(F)F)nc2)n1]

Step 3: Preparation of 4-(tert-Butyl)-6-chloro-N-(6-(difluoromethyl)pyridin-3-yl)pyrimidine-2-carboxamide. 4-(tert-Butyl)-6-chloropyrimidine-2-carbonyl chloride (189 mg, 0.81 mmol) was dissolved in THF (4 mL) and 6-(difluoromethyl)pyridin-3-amine hydrochloride (146 mg, 0.81 mmol) was added, followed by DIEA (424 μL, 2.43 mmol). The resulting mixture was stirred at r.t. for 15 min. and diluted with EtOAc (25 mL) and water (25 mL). The layers were shaken and separated and the organic phase was washed with brine, dried over sodium sulfate, concentrated in vacuo and purified with silica gel using 30% EtOAc/hexanes to provide 4-(tert-butyl)-6-chloro-N-(6-(difluoromethyl)pyridin-3-yl)pyrimidine-2-carboxamide (120 mg, 0.35 mmol, 43%) as a white, amorphous solid. LRMS (APCI) m/z 341.1 (M+H).

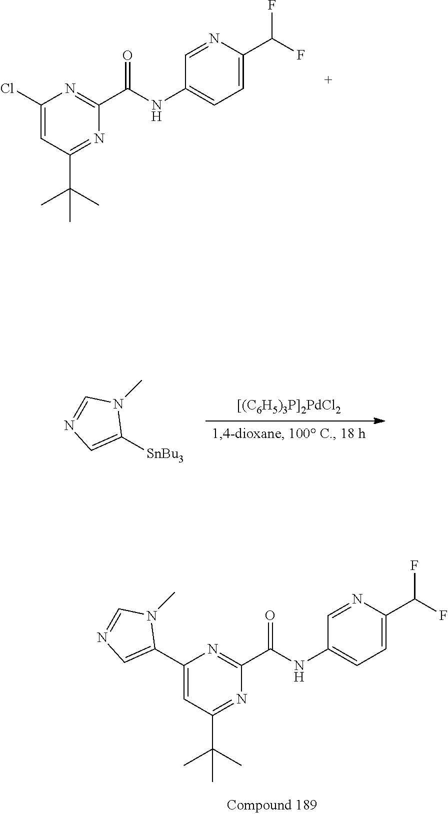

Step 4: Preparation of 4-(tert-Butyl)-N-(6-(difluoromethyl)pyridin-3-yl)-6-(1-methyl-1H-imidazol-5-yl)pyrimidine-2-carboxamide. 4-(tert-Butyl)-6-chloro-N-(6-(difluoromethyl)pyridin-3-yl)pyrimidine-2-carboxamide (62 mg, 0.18 mmol) was combined with trans-dichlorobis(triphenylphosphine)palladium(II) (13 mg, 0.02 mmol) and 1,4-dioxane (4 mL). 1-Methyl-5-(tributylstannyl)-1H-imidazole (68 mg, 0.18 mmol) was added and the mixture was heated in an oil bath at 100° C. for 18 h. The 1,4-dioxane was evaporated under reduced pressure and the product was purified with reverse phase HPLC with a 40 minute gradient from 5-100% ACN/water with 0.1% formic acid in both phases (Phenomenex Gemini 5 micron C18 Axia pack 150×21.2 mm column) to provide 4-(tert-butyl)-N-(6-(difluoromethyl)pyridin-3-yl)-6-(1-methyl-1H-imidazol-5-yl)pyrimidine-2-carboxamide (28 mg, 0.07 mmol, 40%) as a white solid. LRMS (APCI) m/z 387.1 (M+H). ¹H NMR (400 MHz, Methanol-d₄) δ 8.96 (s, 1H), 8.41 (d, J=8.6 Hz, 1H), 7.94-7.74 (m, 3H), 7.64 (d, J=8.6 Hz, 1H), 6.62 (t, J=55.3 Hz, 1H), 4.09 (s, 3H), 1.38 (s, 9H).

Compound 190 was prepared using the methods provided in the table below.

| Compound Number | Method of Preparation |
|---|---|
| 190 | Beginning with methyl 4-(tert-Butyl)-6- |
| | chloropyrimidine-2-carboxylate, prepared in same |
| | fashion Compound 189 |

### Example V

### Synthesis of Compound 191

### Preparation of N-((1r,4r)-4-methoxycyclohexyl)-4-methyl-6-(1-methyl-1H-imidazol-5-yl)pyrimidine-2-carboxamide

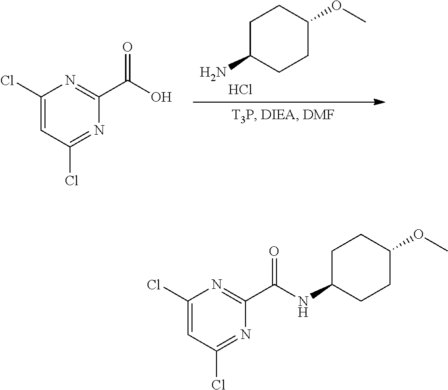

Step 1: Preparation of 4,6-Dichloro-N-((1r,4r)-4-methoxycyclohexyl)pyrimidine-2-carboxamide. To a stirred solution of 4,6-dichloropyrimidine-2-carboxylic acid (980 mg, 5.08 mmol) in DMF (10 mL) were added (1r,4r)-4-methoxycyclohexan-1-amine hydrochloride (1.01 g, 6.09 mmol), T₃P (4.85 g, 7.62 mmol, 50% in EtOAc) and DIEA (2.65, 15.24 mmol). The resulting mixture was stirred at r.t. overnight, water (20 mL) was added and the mixture was extracted twice with EtOAc (20 mL). The organic layers were combined, washed with brine (20 mL), dried over anhydrous Na₂SO₄, concentrated under reduced pressure and purified by silica gel column chromatography using 10% MeOH/DCM to afford 4,6-dichloro-N-[(1r,4r)-4-methoxycyclohexyl]pyrimidine-2-carboxamide (1.10 g, 3.63 mmol, 71% as a yellow solid. LRMS (ES) m/z 304 (M+H).

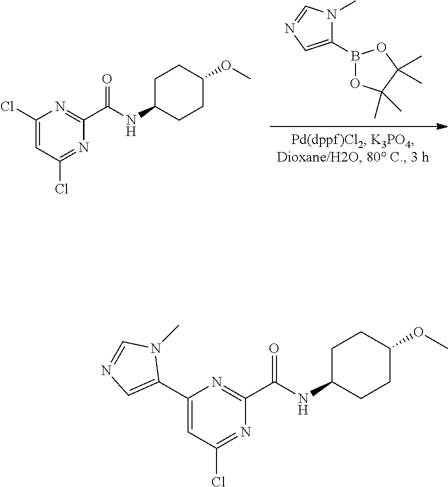

Step 2: Preparation of 4-Chloro-N-((1r,4r)-4-methoxycyclohexyl)-6-(1-methyl-1H-imidazol-5-yl)pyrimidine-2-carboxamide. Prepared in an oil bath at 80° C. for 3 h using same Suzuki coupling procedure as described for Compound 59 to provide 4-chloro-N-((1r,4r)-4-methoxycyclohexyl)-6-(1-methyl-1H-imidazol-5-yl)pyrimidine-2-carboxamide (360 mg, 1.03 mmol, 52% yield) as a yellow solid. LRMS (ESI) m/z 350 (M+H).

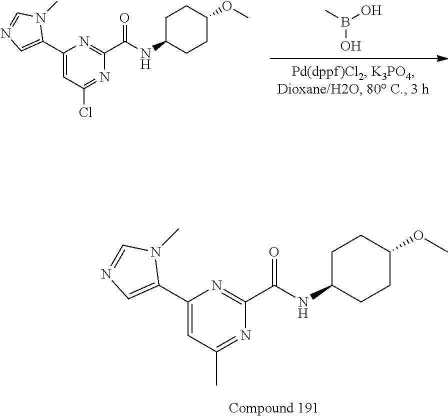

Step 3: Preparation of N-((1r,4r)-4-methoxycyclohexyl)-4-methyl-6-(1-methyl-1H-imidazol-5-yl)pyrimidine-2-carboxamide. To a stirred solution of 4-chloro-6-(3-methylimidazol-4-yl)-N-[(1r,4r)-4-methoxycyclohexyl]pyrimidine-2-carboxamide (100 mg, 0.286 mmol) and methylboronic acid (26 mg, 0.434 mmol) in dioxane (2 mL) and water (0.2 mL) were added Pd(dppf)Cl₂ (21 mg, 0.029 mmol) and K₃PO₄ (121 mg, 0.57 mmol). The resulting mixture was stirred at 80° C. for 5 h under a nitrogen atmosphere. The mixture was cooled to r.t., filtered to remove solids, concentrated under reduced pressure, and purified by silica gel column chromatography using 10% MeOH/DCM followed by C18 column chromatography using water (0.05% NH₄HCO₃)/MeCN (2:1) to afford N-((1r,4r)-4-methoxycyclohexyl)-4-methyl-6-(1-methyl-1H-imidazol-5-yl)pyrimidine-2-carboxamide (33 mg, 0.100 mmol, 35%) as a white solid. LRMS (ES) m/z 330 (M+H). ¹H NMR (400 MHz, DMSO-d₆) δ 8.37 (d, J=8.3 Hz, 1H), 7.91-7.83 (m, 3H), 4.05 (s, 3H), 3.82-3.70 (m, 1H), 3.24 (s, 3H), 3.11 (td, J=10.3, 5.1 Hz, 1H), 2.54 (s, 3H), 2.02 (d, J=12.4 Hz, 2H), 1.87 (d, J=12.4 Hz, 2H), 1.51-1.35 (m, 2H), 1.30-1.16 (m, 2H).

### Example W

### Synthesis of Compound 192

### Preparation of 4-methoxy-N-((1r,4r)-4-methoxycyclohexyl)-6-(1-methyl-1H-imidazol-5-l yl)pyrimidine-2-carboxamide

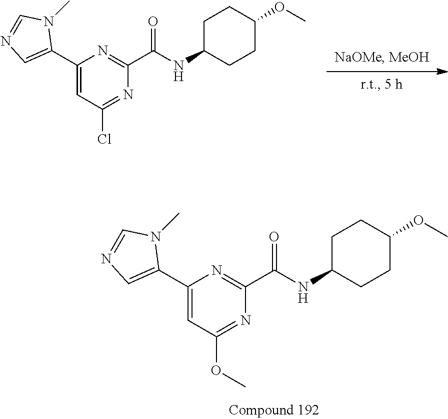

Preparation of 4-Methoxy-N-((1r,4r)-4-methoxycyclohexyl)-6-(1-methyl-1H-imidazol-5-yl)pyrimidine-2-carboxamide. To a stirred solution of 4-chloro-6-(3-methylimidazol-4-ye-N-[(1r,4r)-4-methoxycyclohexyl]pyrimidine-2-carboxamide (90 mg, 0.257 mmol) in MeOH (2 mL) was added NaOMe (0.128 mL of 4 M, 0.512 mmol). The resulting mixture was stirred at r.t. for 5 h and then concentrated under reduced pressure. The product was purified with reverse phase HPLC using the following conditions: (SHIMADZU HPLC) YMC-Actus Triart C18 ExRS column, 30*150 mm, 5 μm; mobile phase: water (10 mmol/L NH₄HCO₃+0.1% NH₃.H₂O) and ACN (18% ACN up to 48% in 8 min) to afford 4-methoxy-N-((1r,4r)-4-methoxycyclohexyl)-6-(1-methyl-1H-imidazol-5-yl)pyrimidine-2-carboxamide (29 mg, 0.084 mmol, 33%) as a yellow solid. LRMS (ES) m/z 346 (M+H). ¹H NMR (400 MHz, DMSO-d₆) δ 8.34 (d, J=8.3 Hz, 1H), 7.86 (q, J=1.2 Hz, 2H), 7.34 (s, 1H), 4.02 (s, 6H), 3.82-3.68 (m, 1H), 3.24 (s, 3H), 3.12 (tt, J=10.3, 4.0 Hz, 1H), 2.06-1.97 (m, 2H), 1.91-1.83 (m, 2H), 1.45 (qd, J=13.0, 3.3 Hz, 2H), 1.31-1.16 (m, 2H).

Compound 201 was prepared using the methods provided in the table below.

| Compound Number | Method of Preparation |
|---|---|
| 201 | Prepared in the same fashion as Compound 192 |

### Example X

### Synthesis of Compound 202

### Preparation of N-(6-(difluoromethyl)pyridin-3-yl)-4-methyl-6-(1-methyl-1H-imidazol-5-yl) pyrimidine-2-carboxamide

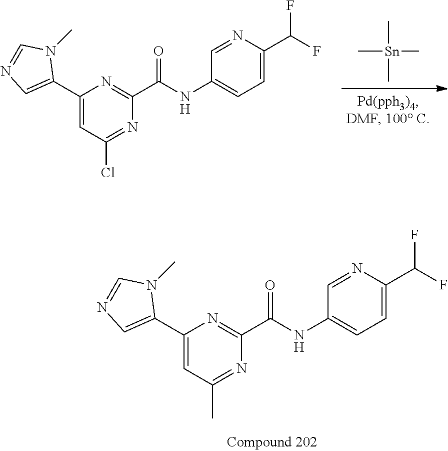

Preparation of N-(6-(Difluoromethyl)pyridin-3-yl)-4-methyl-6-(1-methyl-1H-imidazol-5-yl)pyrimidine-2-carboxamide. To a solution of 4-chloro-N-[6-(difluoromethyl)pyridin-3-yl]-6-(3-methylimidazol-4-yl)pyrimidine-2-carboxamide (95 mg, 0.26 mmol) in DMF (1 mL) were added Sn(CH₃)₄ (47 mg, 0.26 mmol) and Pd(PPh₃)₄ (60 mg, 0.052 mmol) at r.t. under a nitrogen atmosphere. The resulting mixture was stirred at 105° C. for 3 h, cooled to r.t. and concentrated under reduced pressure. The product was purified by C18 column chromatography using water (0.05% NH₄HCO₃)/CH₃CN (4:1) to afford N-(6-(difluoromethyl)pyridin-3-yl)-4-methyl-6-(1-methyl-1H-imidazol-5-yl)pyrimidine-2-carboxamide (28 mg, 0.081 mmol, 31%) as a white solid. LRMS (ES) m/z 345 [M+H]. ¹H NMR (400 MHz, DMSO-d₆) δ 10.97 (s, 1H), 9.11 (d, J=2.4 Hz, 1H), 8.50 (dd, J=8.5, 2.5 Hz, 1H), 8.00-7.92 (m, 3H), 7.76 (d, J=8.5 Hz, 1H), 6.95 (t, J=55.1 Hz, 1H), 4.11 (s, 3H), 2.62 (s, 3H).

### Example Y

### Synthesis of Compound 203

### Preparation of 4-cyclopropyl-N-((1r,4r)-4-methoxycyclohexyl)-6-(1-methyl-1H-imidazol-5-yl)pyrimidine-2-carboxamide

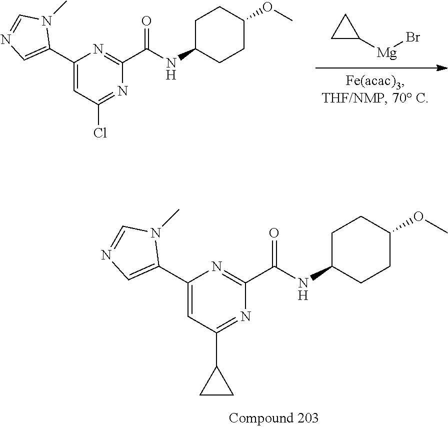

Preparation of 4-Cyclopropyl-N-((1r,4r)-4-methoxycyclohexyl)-6-(1-methyl-1H-imidazol-5-yl)pyrimidine-2-carboxamide. To a stirred solution of 4-chloro-6-(3-methylimidazol-4-yl)-N-[(1r,4r)-4-methoxycyclohexyl]pyrimidine-2-carboxamide (130 mg, 0.372 mmol) and Fe(acac)₃ (26 mg, 0.074 mmol) in THF (3 mL) and NMP (0.5 mL) was added bromo(cyclopropyl)magnesium (0.74 mL, 0.744 mmol, 2 equiv, 1M in THF) dropwise under nitrogen atmosphere. The resulting mixture was stirred at 70° C. for 18 h, cooled to r.t and purified twice by C18 column chromatography using water (0.05% NH₄HCO₃)/MeCN (2:1) followed by SFC with the following conditions: Green Sep Naphthyl column, 3*25 cm, 5 μm; Mobile Phase A: CO₂, Mobile Phase B: MeOH (0.5% 2 M NH₃-MeOH); Flow rate: 75 mL/min; isocratic gradient 45% B to afford 4-cyclopropyl-N-((1r,4r)-4-methoxycyclohexyl)-6-(1-methyl-1H-imidazol-5-yl)pyrimidine-2-carboxamide (23 mg, 17%) as a yellow solid. LRMS (ES) m/z 356 (M+H). ¹H NMR (400 MHz, DMSO-d₆) δ 8.27 (d, J=8.2 Hz, 1H), 7.88 (s, 2H), 7.82 (s, 1H), 4.03 (s, 3H), 3.88-3.63 (m, 1H), 3.24 (s, 3H), 3.17-3.07 (m, 1H), 2.19 (dq, J=10.0, 4.0, 3.3 Hz, 1H), 2.01 (d, J=11.8 Hz, 2H), 1.86 (d, J=13.3 Hz, 2H), 1.44 (dt, J=13.4, 10.6 Hz, 2H), 1.24 (t, J=12.8 Hz, 2H), 1.23-1.06 (m, 4H).

Compounds 206 and 209 were prepared using the methods provided in the table below.

| Compound Number | Method of Preparation |
|---|---|
| 206 | Prepared in same fashion as Compound 202 with |
| | Stille coupling using |
| | tributyl(cyclopropyl)stannane in last step |
| 209 | Beginning with methyl 4-chloropyrimidine-2- |
| | carboxylate, Stille coupling followed by ester |
| | hydrolysis, acyl chloride synthesis and amide |
| | bond formation using same procedures as described |
| | for Compound 189 |

### Example Z

### Synthesis of Compound 213

### Preparation of 4-(1-methyl-1H-imidazol-5-yl)-N-((1r,3r)-3-phenylcyclobutyl)pyrimidine-2-carboxamide

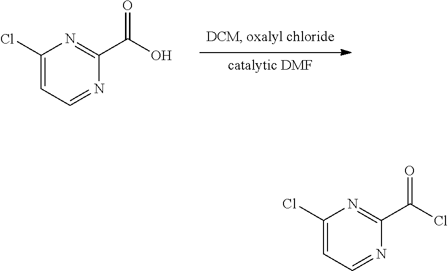 [SMILES: O=C(O)c1nccc(Cl)n1 ; "DCM, oxalyl chloride" ; "catalytic DMF" ; O=C(Cl)c1nccc(Cl)n1]

Step 1: Preparation of 4-Chloropyrimidine-2-carbonyl chloride. Prepared using the same procedure as described for Compound 189 to give 4-chloropyrimidine-2-carbonyl chloride (446 mg, 2.52 mmol, quantitative yield) as a glassy solid.

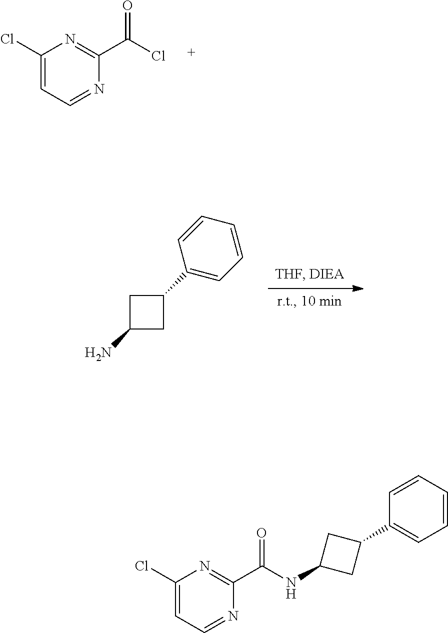 [SMILES: O=C(Cl)c1nccc(Cl)n1 ; N[C@H]1C[C@H](c2ccccc2)C1 ; "THF, DIEA" ; "r.t., 10 min" ; O=C(N[C@H]1C[C@H](c2ccccc2)C1)c1nccc(Cl)n1]

Step 2: Preparation of 4-Chloro-N-((1r,3r)-3-phenylcyclobutyl)pyrimidine-2-carboxamide. Prepared using the same procedure as described for Compound 189 to provide 4-chloro-N-((1r,3r)-3-phenylcyclobutyl)pyrimidine-2-carboxamide (364 mg, 1.27 mmol) as an off white solid. LRMS (APCI) m/z 288.0 (M+H).

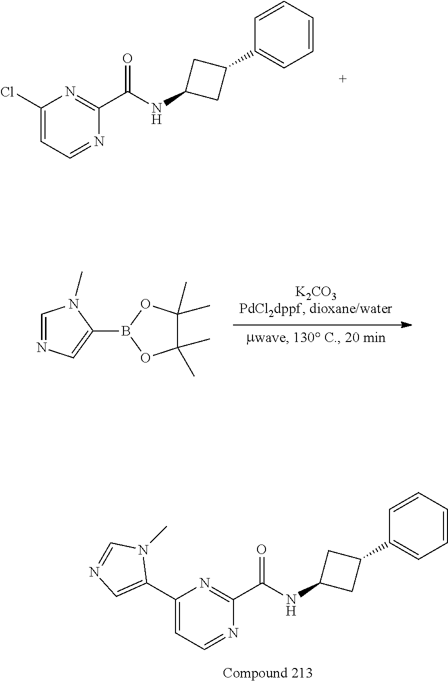 [SMILES: O=C(N[C@H]1C[C@H](c2ccccc2)C1)c1nccc(Cl)n1 ; Cn1cncc1B1OC(C)(C)C(C)(C)O1 ; "K2CO3PdCl2dppf, dioxane/water" ; "mwave, 130° C., 20 min" ; Cn1cncc1-c1ccnc(C(=O)N[C@H]2C[C@H](c3ccccc3)C2)n1]

Step 3: Preparation of 4-(1-Methyl-1H-imidazol-5-yl)-N-((1r,3r)-3-phenylcyclobutyl)pyrimidine-2-carboxamide. 4-chloro-N-((1r,3r)-3-phenylcyclobutyl)pyrimidine-2-carboxamide (182 mg, 0.63 mmol) was combined with 1-methyl-5-(4,4,5,5-tetramethyl-1,3,2-dioxaborolan-2-yl)-1H-imidazole (145 mg, 0.70 mmol), potassium carbonate (175 mg, 1.27 mmol) and PdCl₂dppf (44 mg, 0.063 mmol). To the solids was added 1,4-dioxane (3 mL) and water (1 mL). The resulting mixture was heated in a microwave at 130° C. for 20 min. The solvents were evaporated under reduced pressure and the product was purified using reverse phase HPLC with a 40 minute gradient from 5-100% ACN/water with 0.1% formic acid in both phases (Phenomenex Gemini 5 micron C18 Axia pack 150×21.2 mm column) to provide 4-(1-methyl-1H-imidazol-5-yl)-N-((1r,3r)-3-phenylcyclobutyl)pyrimidine-2-carboxamide LRMS (APCI) m/z 334.1 (M+H). ¹H NMR (400 MHz, Methanol-d₄) δ 8.86 (d, J=5.5 Hz, 1H), 7.98-7.81 (m, 3H), 7.42-7.29 (m, 4H), 7.22 (td, J=6.1, 2.8 Hz, 1H), 4.72 (p, J=7.3 Hz, 1H), 4.22 (s, 3H), 3.70 (td, J=9.3, 4.7 Hz, 1H), 2.78-2.54 (m, 4H).

Compounds 214 and 218 were prepared using the methods provided in the table below.

| Compound Number | Method of Preparation |
|---|---|
| 214 | Prepared in same fashion as Compound 213 |
| 218 | Prepared in same fashion as Compound 213 |

### Example AA

### Synthesis of Compound 220

### Preparation of 4-(1-methyl-1H-imidazol-5-yl)-N-((1r,3r)-3-phenoxycyclobutyl)pyrimidine-2-carboxamide

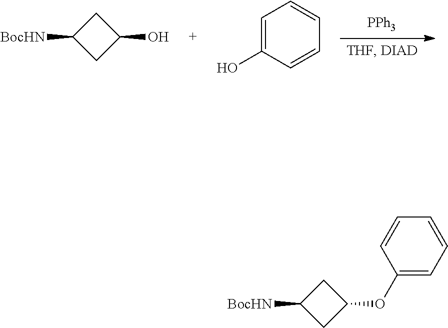

Step 1: Preparation of tert-Butyl ((1r,3r)-3-phenoxycyclobutyl)carbamate. tert-butyl ((1s,3s)-3-hydroxycyclobutyl)carbamate (785 mg, 4.19 mmol) was combined with triphenylphosphine (1.649 g, 6.29 mmol) and phenol (473 mg, 5.03 mmol). THF (25 mL) was added, followed by diisopropyl azodicarboxylate (1.238 mL, 6.29 mmol). The resulting mixture was heated in an oil bath at 50° C. for 18 h, cooled to r.t. and concentrated. The remaining oil was partitioned between 1 M aq. KOH (30 mL) and DCM (80 mL). The organic phase was dried over sodium sulfated, concentrated under reduced pressure and purified with silica gel using 15% ethyl acetate/hexanes to provide tert-butyl ((1r,3r)-3-phenoxycyclobutyl)carbamate (358 g, 1.36 mmol, 32%) as a colorless viscous oil. LRMS (APCI) m/z 208.1 (M+H-56). ¹H NMR (400 MHz, DMSO-d₆) δ 7.35-7.17 (m, 3H), 6.92 (t, J=7.3 Hz, 1H), 6.80 (d, J=8.1 Hz, 2H), 4.84-4.72 (m, 1H), 4.12-4.02 (m, 1H), 2.41-2.23 (m, 4H), 1.39 (s, 9H). (Q. Zhange et al./European Journal of Medicinal Chemistry 187 (2020) 111973 for ¹H NMR of cis vs. trans diastereomers).

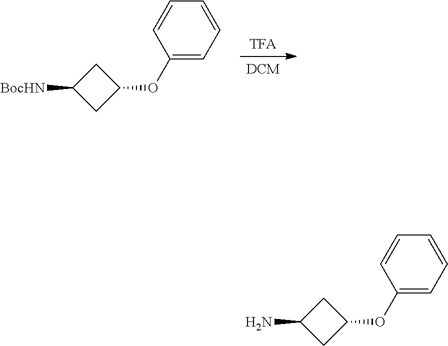

Step 2: Preparation of (1r,3r)-3-Phenoxycyclobutan-1-amine TFA. Same Boc removal procedure as described for Compound 44 to provide (1r,3r)-3-phenoxycyclobutan-1-amine TFA (375 mg, 1.36 mmol, quantitative yield) as a glassy solid. LRMS (APCI) m/z 164.1 (M+H).

Step 3: Preparation of 4-(1-methyl-1H-imidazol-5-yl)-N-((1r,3r)-3-phenoxycyclobutyl)pyrimidine-2-carboxamide. Using (1r,3r)-3-phenoxycyclobutan-1-amine TFA, prepared in the same fashion as Compound 213 to provide 4-(1-methyl-1H-imidazol-5-yl)-N-((1r,3r)-3-phenoxycyclobutyl)pyrimidine-2-carboxamide (23 mg, 0.066 mmol, 67%) as a white solid. LRMS (APCI) m/z 334.1 (M+H). ¹H NMR (400 MHz, Methanol-d₄) δ 8.86 (d, J=5.4 Hz, 1H), 7.97-7.85 (m, 3H), 7.28 (t, J=7.7 Hz, 2H), 6.93 (d, J=7.7 Hz, 1H), 6.85 (d, J=8.1 Hz, 2H), 5.01-4.90 (m, 1H), 4.80-4.68 (m, 1H), 4.21 (s, 3H), 2.76-2.58 (m, 4H).

Compound 222 was prepared using the methods provided in the table below.

| Compound Number | Method of Preparation |
|---|---|
| 222 | Beginning with (1r,3r)-3-aminocyclobutan-1-ol, |
| | prepared in the same fashion as Compound 351 |

### Example AB

### Synthesis of Compound 224

### Preparation of N-(6-(difluoromethyl)pyridin-3-yl)-4-(2-methoxyethoxy)-6-(1-methyl-1H-imidazol-5-yl)pyrimidine-2-carboxamide

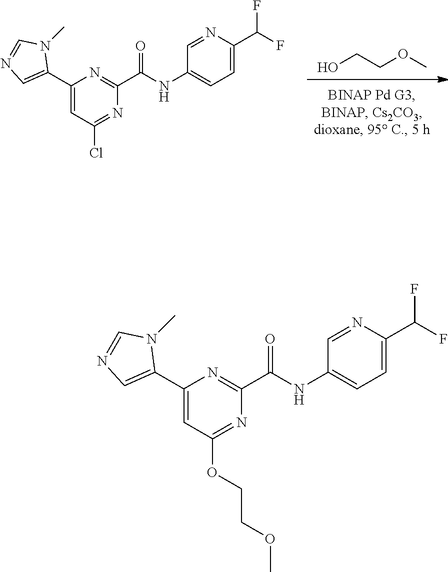

Preparation of N-(6-(Difluoromethyl)pyridin-3-yl)-4-(2-methoxyethoxy)-6-(1-methyl-1H-imidazol-5-yl)pyrimidine-2-carboxamide. To 4-chloro-N-[6-(difluoromethyl)pyridin-3-yl]-6-(3-methylimidazol-4-yl)pyrimidine-2-carboxamide (90 mg, 0.25 mmol) and 2-methoxyethanol (28 mg, 0.37 mmol) in dioxane (5 mL) were added rac-BINAP-PD-G3 (12 mg, 0.012 mmol) and Cs₂CO₃ (161 mg, 0.49 mmol). The resulting mixture was stirred at 110° C. for 18 h under a nitrogen atmosphere. The mixture was allowed to cool to r.t., filtered to remove solids, concentrated under reduced pressure and purified by C18 column chromatography using water (0.05% NH₄HCO₃)/MeCN (2:1) as the mobile phase followed by reverse phase HPLC with the following conditions: (SHIMADZU HPLC) XBridge Prep OBD C18 Column, 30*150 mm, 5 μm; mobile phase, Water (10 mmol/L NH₄HCO₃) and ACN (18% ACN up to 48% in 8 min) to afford N-(6-(difluoromethyl)pyridin-3-yl)-4-(2-methoxyethoxy)-6-(1-methyl-1H-imidazol-5-yl)pyrimidine-2-carboxamide (9 mg, 0.022 mmol, 9%) as a white solid. LRMS (ES) m/z 405 (M+H). ¹H NMR (400 MHz, Methanol-d₄) δ 9.06 (d, J=2.5 Hz, 1H), 8.51 (dd, J=8.6, 2.5 Hz, 1H), 7.85 (s, 1H), 7.81 (d, J=1.2 Hz, 1H), 7.75 (d, J=8.5 Hz, 1H), 7.34 (s, 1H), 6.73 (t, J=55.3 Hz, 1H), 4.77-4.71 (m, 2H), 4.16 (s, 3H), 3.85-3.78 (m, 2H), 3.43 (s, 3H).

Compounds 231-235, 240-243, and 246 were prepared using the methods provided in the table below.

| Compound Number | Method of Preparation |
|---|---|
| 231 | Prepared in same fashion as Compound 192 |
| 232 | Prepared in same fashion as Compound 191 |
| 233 | Prepared in same fashion as Compound 224 |
| 234 | Beginning with 4,6-dichloropyrimidine-2- |
| | carboxylic acid, amide bond formation as |
| | described for Compound 191 followed by |
| | nucleophilic aromatic substitution with sodium |
| | methoxide as described for Compound 192 and |
| | Suzuki coupling as described for Compound 191 |
| 235 | Beginning with 4,6-dichloropyrimidine-2- |
| | carboxylic acid, amide bond formation as |
| | described for Compound 191 followed by Stille |
| | coupling as described for Compound 202 and |
| | Suzuki coupling as described for Compound 191 |
| 240 | Prepared in same fashion as Compound 213 |
| 241 | Prepared in same fashion as Compound 213 |
| 242 | Prepared in same fashion as Compound 213 |
| 243 | Prepared in same fashion as Compound 213 |
| 246 | Prepared in same fashion as Compound 213 |

### Example AC

### Synthesis of Compound 250

### Preparation of N-(6-(2-hydroxypropan-2-yl)pyridin-3-yl)-4-(1-methyl-1H-imidazol-5-yl) pyrimidine-2-carboxamide

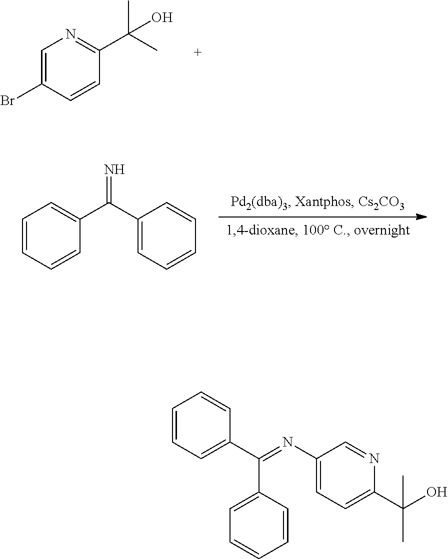 [SMILES: CC(C)(O)c1ccc(Br)cn1 ; N=C(c1ccccc1)c1ccccc1 ; "1,4-dioxane, 100° C., overnight" ; "Pd2(dba)3, Xantphos, Cs2CO3" ; CC(C)(O)c1ccc(N=C(c2ccccc2)c2ccccc2)cn1]

Step 1: Preparation of 2-(5-((Diphenylmethylene)amino)pyridin-2-yl)propan-2-ol. 2-(5-bromopyridin-2-yl)propan-2-ol (3.0 g, 13.88 mmol) was combined with benzophenone imine (2.80 mL, 16.68 mmol), tris(dibenzylideneacetone)dipalladium(0) (1.017 g, 1.11 mmol), (9,9-Dimethyl-9H-xanthene-4,5-diyl)bis(diphenylphosphane) (0.635 g, 1.10 mmol), and cesium carbonate (13.571 g, 41.65 mmol). 1,4-dioxane (30 mL) was added and the resulting mixture was heated in an oil bath at 100° C. for 18 h. The mixture was filtered through celite and the solvent was removed under reduced pressure. The product was purified with silica gel using 20% ethyl acetate/hexanes, providing 2-(5-((diphenylmethylene)amino)pyridin-2-yl)propan-2-ol (3.104 g, 9.81 mmol, 71%) as an orange oil which was used in the subsequent step without additional purification. LRMS (APCI) m/z 317.1 (M+H).

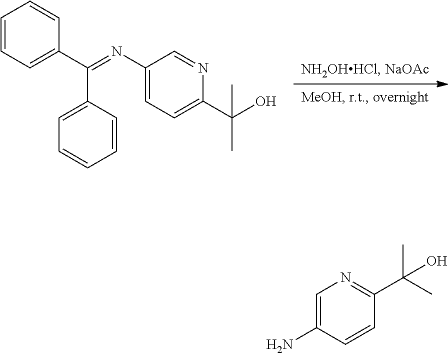

Step 2: Preparation of 2-(5-Aminopyridin-2-yl)propan-2-ol. 2-(5-((diphenylmethylene)amino)pyridin-2-yl)propan-2-ol (3.104 g, 9.81 mmol) was dissolved in methanol. To this mixture was added hydroxylamine hydrochloride (1.022 g, 14.72 mmol) and sodium acetate (1.207 g, 14.72 mmol). The resulting mixture was stirred at r.t. overnight. Additional hydroxylamine hydrochloride (1.022 g, 14.72 mmol) and sodium acetate (1.207 g, 14.72 mmol) were added and the mixture stirred for 2 additional hours. The mixture was diluted with ethyl acetate (150 mL), filtered through celite and the solvent was removed under reduced pressure. The product was purified with silica gel using 20% methanol/DCM providing 2-(5-aminopyridin-2-yl)propan-2-ol (1.193 g, 7.84 mmol, 80%) as a brown oil which was used in subsequent steps without additional purification. LRMS (APCI) m/z 153.1 (M+H).

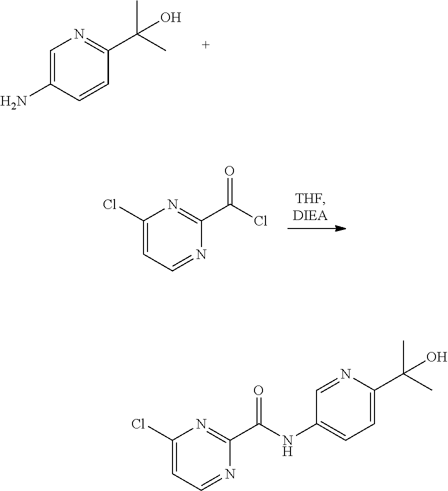 [SMILES: CC(C)(O)c1ccc(N)cn1 ; O=C(Cl)c1nccc(Cl)n1 ; CC(C)(O)c1ccc(NC(=O)c2nccc(Cl)n2)cn1]

Step 3: Preparation of 4-Chloro-N-(6-(2-hydroxypropan-2-yl)pyridin-3-yl)pyrimidine-2-carboxamide. Prepared using same amide bond formation procedure as described for Compound 189 to yield 4-chloro-N-(6-(2-hydroxypropan-2-yl)pyridin-3-yl)pyrimidine-2-carboxamide (145 mg, 0.50 mmol, 51% yield) as a faintly yellow solid. LRMS (APCI) m/z 293.1 (M+H).

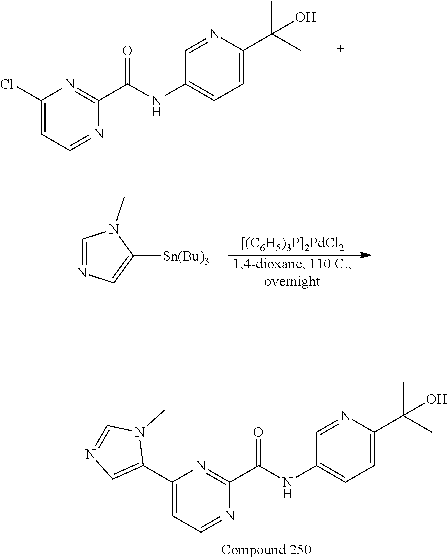

Step 4: Preparation of N-(6-(2-hydroxypropan-2-yl)pyridin-3-yl)-4-(1-methyl-1H-imidazol-5-yl)pyrimidine-2-carboxamide. 4-chloro-N-(6-(2-hydroxypropan-2-yl)pyridin-3-yl)pyrimidine-2-carboxamide (0.145 g, 0.495 mmol), 1-methyl-5-(tributylstannyl)-1H-imidazole (0.166 mL, 0.544 mmol) and trans-dichlorobis(triphenylphosphine)palladium(II) (0.035 g, 0.049 mmol) were dissolved in 1,4-dioxane (5 mL) and heated in an oil bath at 110 C overnight. The reaction was concentrated under reduced pressure and purified twice using reverse phase HPLC with a 40 minute gradient from 0-100% ACN/water (Phenomenex Gemini 5 micron C18 column), yielding N-(6-(2-hydroxypropan-2-yl)pyridin-3-yl)-4-(1-methyl-1H-imidazol-5-yl)pyrimidine-2-carboxamide (0.023 g, 0.069 mmol, 14%) as a white solid. LRMS (APCI) m/z 339.1 (M+H). ¹H NMR (400 MHz, Methanol-d₄) δ 9.07-8.82 (m, 2H), 8.31 (d, J=8.7 Hz, 1H), 7.98 (d, J=5.3 Hz, 3H), 7.75 (d, J=8.6 Hz, 1H), 4.24 (s, 3H), 1.59 (s, 6H).

### Example AD

### Synthesis of (1r,4r)-4-Amino-1-phenylcyclohexan-1-ol and (1s,4s)-4-Amino phenylcyclohexan-1-ol

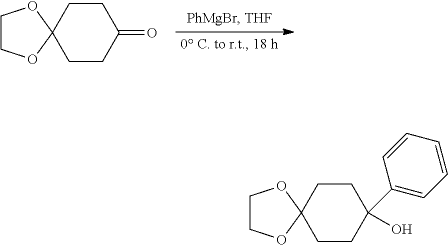 [SMILES: O=C1CCC2(CC1)OCCO2 ; OC1(c2ccccc2)CCC2(CC1)OCCO2]

Step 1: Preparation of 8-Phenyl-1,4-dioxaspiro[4.5]decan-8-ol. 1,4-dioxaspiro[4.5]decan-8-one (2.45 g, 15.7 mmol) was dissolved in THF (25 mL) and cooled to 0° C. with an ice bath. Phenyl magnesium bromide (17.2 mL of 1.0 M in THF, 17.2 mmol) was added using a syringe and the resulting mixture was stirred for 18 h, during which time it was allowed to warm to r.t. The mixture was quenched with saturated aqueous ammonium chloride (30 mL) and diluted with EtOAc (150 mL). The layers were separated and the organic phase was washed with brine, dried over sodium sulfate and concentrated under reduced pressure. The product was purified with silica gel using 60% EtOAc/hexanes to provide 8-Phenyl-1,4-dioxaspiro[4.5]decan-8-ol (1.98 g, 8.47 mmol, 54% yield) as a white solid. ¹H NMR (400 MHz, DMSO-d₆) δ 7.50-7.42 (m, 2H), 7.31 (t, J=7.5 Hz, 2H), 7.20 (t, J=7.3 Hz, 1H), 4.88 (s, 1H), 3.89 (s, 4H), 2.02-1.88 (m, 4H), 1.70-1.60 (m, 2H), 1.58-1.47 (m, 2H).

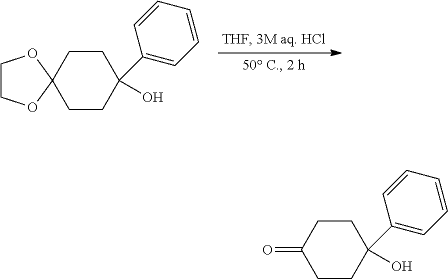 [SMILES: OC1(c2ccccc2)CCC2(CC1)OCCO2 ; O=C1CCC(O)(c2ccccc2)CC1]

Step 2: Preparation of 4-Hydroxy-4-phenylcyclohexan-1-one. 8-phenyl-1,4-dioxaspiro[4.5]decan-8-ol (1.98 g, 8.47 mmol) was dissolved in THF (15 mL) and 3 M aq. HCl (6.0 mL, 18 mmol) was added. The resulting solution was heated in an oil bath at 50° C. for 2 h. It was cooled to r.t., carefully diluted with saturated aqueous NaHCO₃ (50 mL) and EtOAc (75 mL). The layers were separated and the aqueous phase was extracted with additional EtOAc (50 mL). The organic phases were combined, dried over sodium sulfate and concentrated under reduced pressure to provide 4-hydroxy-4-phenylcyclohexan-1-one (1.54 g, 8.12 mmol, 96%) as a white solid. LRMS (APCI) m/z 173.1 (M+H—H₂O).

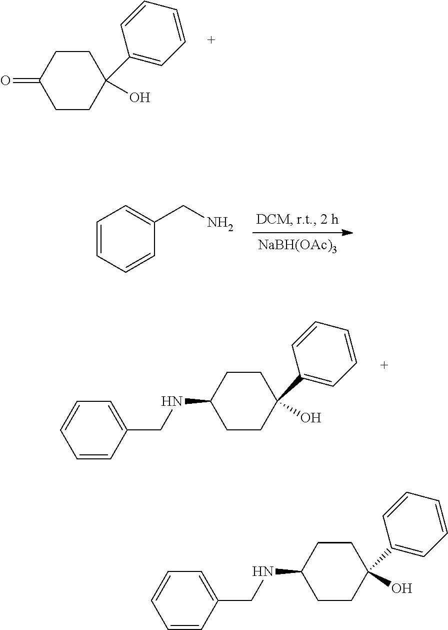 [SMILES: O=C1CCC(O)(c2ccccc2)CC1 ; NCc1ccccc1 ; O[C@]1(c2ccccc2)CC[C@@H](NCc2ccccc2)CC1 ; O[C@]1(c2ccccc2)CC[C@H](NCc2ccccc2)CC1 ; "NaBH(OAc)3"]

Step 3: Preparation of (1r,4r)-4-(Benzylamino)-1-phenylcyclohexan-1-ol and (1s,4s)-4-(benzylamino)-1-phenylcyclohexan-1-ol. 4-hydroxy-4-phenylcyclohexan-1-one (493 mg, 2.59 mmol) was dissolved in DCM (5 mL) and benzyl amine (283 mL) was added followed by NaBH(OAc)₃ (824 mg, 3.89 mmol). The resulting mixture was stirred at r.t. for 2 h. Additional DCM (50 mL) was added and the mixture was washed with saturated aqueous sodium bicarbonate (50 mL), brine, dried over sodium sulfate and concentrated in vacuo. The products were purified with silica gel using 100% ethyl acetate to elute (1r,4r)-4-(benzylamino)-1-phenylcyclohexan-1-ol (179 mg, 0.64 mmol, 25%) as a sticky colorless solid followed by 10% MeOH/DCM to elute (1s,4s)-4-(benzylamino)-1-phenylcyclohexan-1-ol (113 mg, 0.40 mmol, 15%) as a white solid. (1r,4r)-4-(benzylamino)-1-phenylcyclohexan-1-ol: LRMS (APCI) m/z 282.1 (M+H). ¹H NMR (400 MHz, Methanol-d₄) δ 7.62-7.55 (m, 2H), 7.40-7.30 (m, 6H), 7.24 (q, J=7.4 Hz, 2H), 3.78 (s, 2H), 2.80 (tt, J=7.0, 3.8 Hz, 1H), 2.38 (ddd, J=13.0, 8.9, 3.9 Hz, 2H), 2.00 (ddt, J=13.0, 8.5, 3.9 Hz, 2H), 1.63 (ddd, J=13.0, 8.5, 3.9 Hz, 2H), 1.55-1.40 (m, 2H). (1s,4s)-4-(benzylamino)-1-phenylcyclohexan-1-ol: LRMS (APCI) m/z 282.1 (M+H).

¹H NMR (400 MHz, Methanol-d₄) δ 7.41-7.30 (m, 2H), 7.30-7.11 (m, 7H), 7.07 (t, J=7.3 Hz, 1H), 3.72 (s, 2H), 2.58-2.47 (m, 1H), 1.82-1.57 (m, 8H).

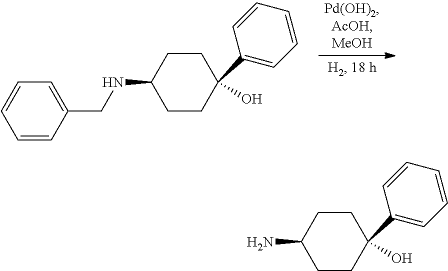 [SMILES: O[C@]1(c2ccccc2)CC[C@@H](NCc2ccccc2)CC1 ; "H2, 18 h" ; N[C@H]1CC[C@@](O)(c2ccccc2)CC1]

Step 4a: Preparation of (1r,4r)-4-Amino-1-phenylcyclohexan-1-ol. (1r,4r)-4-(benzylamino)-1-phenylcyclohexan-1-ol (179 mg, 0.64 mmol) was dissolved in MeOH (6 mL) and AcOH (20 μL) was added, followed by Pd(OH)₂ on carbon (125 mg, 1.0 mmol). The resulting heterogeneous mixture was stirred under 70 psi H₂ for 18 h. The mixture was filtered through a syringe filter and concentrated under reduced pressure to provide (1r,4r)-4-Amino phenylcyclohexan-1-ol (121 mg, 0.63 mmol, quantitative yield) as a white solid. LRMS (APCI) m/z 192.1 (M+H).

Step 4b: Preparation of (1s,4s)-4-Amino-1-phenylcyclohexan-1-ol: Beginning with (1s,4s)-4-(benzylamino)-1-phenylcyclohexan-1-ol (119 mg, 0.42 mmol), synthesized using same procedure as (1r,4r)-4-amino-1-phenylcyclohexan-1-ol to provide (1s,4s)-4-amino-1-phenylcyclohexan-1-ol (80 mg, 0.42 mmol, quantitative yield) as a white solid. LRMS (APCI) m/z 192.1 (M+H).

### Example AE

### Synthesis of Compound 256

### Preparation of N-((1r,4r)-4-hydroxycyclohexyl)-4-(1-methyl-1H-imidazol-5-yl)pyrimidine-2-carboxamide

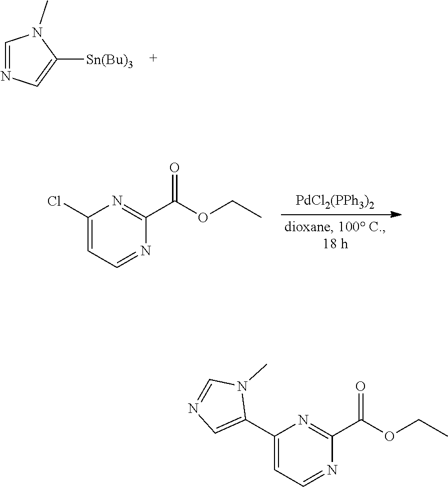

Step 1: Preparation of Ethyl 4-(1-methyl-1H-imidazol-5-yl)pyrimidine-2-carboxylate. Beginning with ethyl 4-chloropyrimidine-2-carboxylate, prepared using same Stille coupling procedure as described for Compound 250.

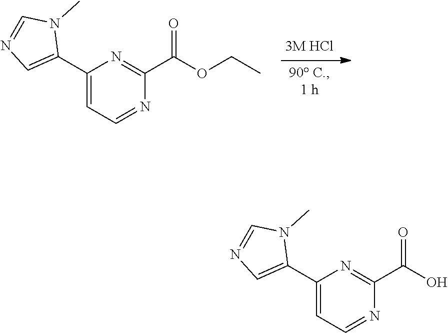 [SMILES: CCOC(=O)c1nccc(-c2cncn2C)n1 ; "3M HCl" ; Cn1cncc1-c1ccnc(C(=O)O)n1]

Step 2: Preparation of 4-(1-Methyl-1H-imidazol-5-yl)pyrimidine-2-carboxylic acid HCl. To ethyl 4-(1-methyl-1H-imidazol-5-yl)pyrimidine-2-carboxylate (0.734 g, 3.16 mmol) was added 3 M HCl. The mixture was heated in an oil bath at 90° C. for 1 h and concentrated to yield the hydrochloride salt of 4-(1-methyl-1H-imidazol-5-yl)pyrimidine-2-carboxylic acid as a tan solid (0.759 g, 3.16 mmol, quantitative yield) which was used in subsequent steps without further purification. LRMS (APCI) m/z 205.1 (M+H).

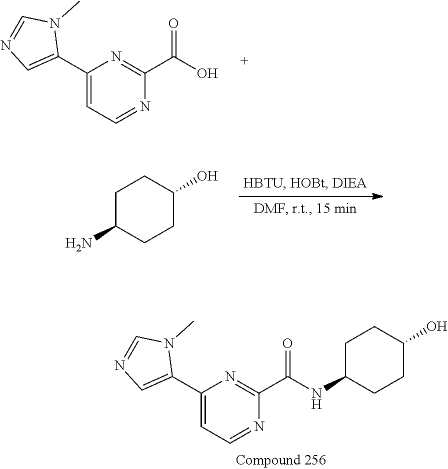 [SMILES: Cn1cncc1-c1ccnc(C(=O)O)n1 ; N[C@H]1CC[C@H](O)CC1 ; "HBTU, HOBt, DIEA" ; Cn1cncc1-c1ccnc(C(=O)N[C@H]2CC[C@H](O)CC2)n1]

Step 3: Preparation of N-((1r,4r)-4-hydroxycyclohexyl)-4-(1-methyl-1H-imidazol-5-yl)pyrimidine-2-carboxamide. 4-(1-methyl-1H-imidazol-5-yl)pyrimidine carboxylic acid HCl (0.059 g, 0.245 mmol) was combined with (1r,4r)-4-aminocyclohexan-1-ol hydrochloride (0.041 g, 0.269 mmol), HBTU (0.139 g, 0.367 mmol), and HOBt (0.050 g, 0.367 mmol) and dissolved in DMF (1.5 mL). DIEA (0.213 mL, 1.224 mmol) was added and the mixture was stirred at room temperature for 15 minutes. The reaction was purified twice with reverse phase HPLC with a 40 minute gradient from 0-100% ACN/water (Phenomenex Gemini 5 micron C18 column), yielding N-((1r,4r)-4-hydroxycyclohexyl)-4-(1-methyl-1H-imidazol-5-yl)pyrimidine-2-carboxamide (0.015 g, 0.05 mmol, 21%) as a white solid. LRMS (APCI) m/z 302.1 (M+H). ¹H NMR (400 MHz, Methanol-d₄) δ 8.84 (d, 1H), 8.63 (d, J=8.5 Hz, 1H), 8.03 (d, J=64.6 Hz, 2H), 4.20 (s, 3H), 3.98-3.86 (m, 1H), 3.68-3.55 (m, 1H), 2.08-2.01 (m, 4H), 1.63-1.35 (m, 4H).

Compounds 252-255 and 259 were prepared using the methods provided in the table below.

| Compound Number | Method of Preparation |
|---|---|
| 252 | Beginning with ethyl 4-chloropyrimidine-2- |
| | carboxylate, Stille coupling, ester hydrolysis |
| | and amide bond formation using (1s,4s)-4-amino-1- |
| | phenylcyclohexan-1-ol as described for Compound |
| | 256 |
| 253 | Beginning with ethyl 4-chloropyrimidine-2- |
| | carboxylate, Stille coupling, ester hydrolysis |
| | and amide bond formation using (1r,4r)-4-amino-1- |
| | phenylcyclohexan-1-ol as described for Compound |
| | 256 |
| 254 | Beginning with 4-chloropyrimidine-2-carboxylic |
| | acid and 2-((1r,4r)-4-aminocyclohexyl)propan-2- |
| | ol, amide bond formation and Stille coupling were |
| | performed in the same fashion as Compound 250 |
| 255 | Beginning with (1r,4r)-4-ethoxycyclohexan-1-amine |
| | and 4-(1-methyl-1H- imidazol-5-yl)pyrimidine-2- |
| | carboxylic acid, amide coupling performed in the |
| | same fashion as Compound 256 |
| 259 | Beginning with 4-(1-methyl-1H-imidazol-5- |
| | yl)pyrimidine-2-carboxylic acid HCl and ((1r,4r)- |
| | 4-aminocyclohexyl)methanol, amide bond formation |
| | performed in the same fashion as Compound 256 |

### Example AF

### Synthesis of Compound 264

### Preparation of 4-(1-methyl-1H-imidazol-5-yl)-N-((1r,4r)-4-((2,2,2-trifluoroethyl)amino)cyclohexyl)pyrimidine-2-carboxamide

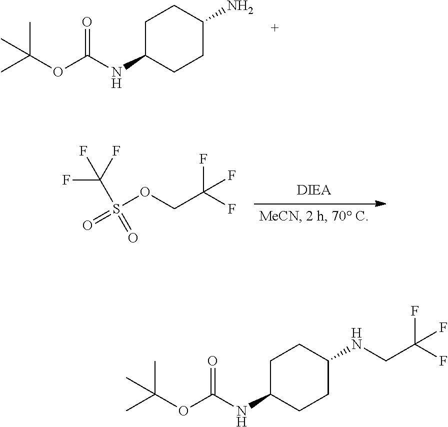 [SMILES: CC(C)(C)OC(=O)N[C@H]1CC[C@H](N)CC1 ; O=S(=O)(OCC(F)(F)F)C(F)(F)F ; "DIEA" ; CC(C)(C)OC(=O)N[C@H]1CC[C@H](NCC(F)(F)F)CC1]

Step 1: Preparation of tert-Butyl ((1r,4r)-4-((2,2,2-trifluoroethyl)amino)cyclohexyl)carbamate: tert-butyl ((1r,4r)-4-aminocyclohexyl)carbamate (2.00 g, 9.33 mmol) and 2,2,2-trifluoroethyl trifluoromethanesulfonate (1.61 mL, 11.20 mmol) were combined with N, N-diisopropylethylamine (4.88 mL, 28.0 mmol) and acetonitrile (16 mL) and heated at 70° C. for 2 h. The reaction mixture was concentrated under reduced pressure and then partitioned between EtOAc (150 mL) and water (100 mL). The organic phase was dried over sodium sulfate, concentrated in vacuo, and purified with silica gel using 50% ethyl acetate/hexanes, providing tert-butyl ((1r,4r)-4-((2,2,2-trifluoroethyl)amino)cyclohexyl)carbamate (2.10 g, 7.10 mmol, 76%) which was used in the subsequent step without additional purification. LRMS (APCI) m/z 297.2 (M+H).

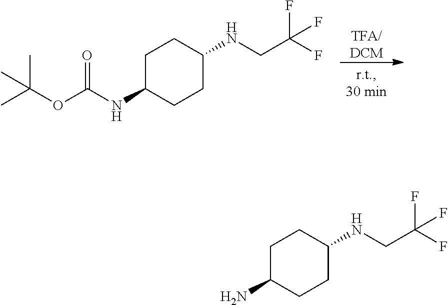 [SMILES: CC(C)(C)OC(=O)N[C@H]1CC[C@H](NCC(F)(F)F)CC1 ; "TFA/DCM" ; N[C@H]1CC[C@H](NCC(F)(F)F)CC1]

Step 2: Preparation of (1r,4r)-N¹-(2,2,2-trifluoroethyl)cyclohexane-1,4-diamine: tert-butyl ((1r,4r)-4-((2,2,2-trifluoroethyl)amino)cyclohexyl)carbamate (2.10 g, 7.10 mmol) was dissolved in trifluoroacetic acid (125 mL) and DCM (125 mL) and stirred at r.t. for 30 min. The reaction mixture was concentrated under reduced pressure and dried under high vacuum to provide (1r,4r)-N¹-(2,2,2-trifluoroethyl)cyclohexane-1,4-diamine TFA (2.20 g. 7.10 mmol, quantitative yield) as a sticky solid. LRMS (APCI) m/z 197.1 (M+H).

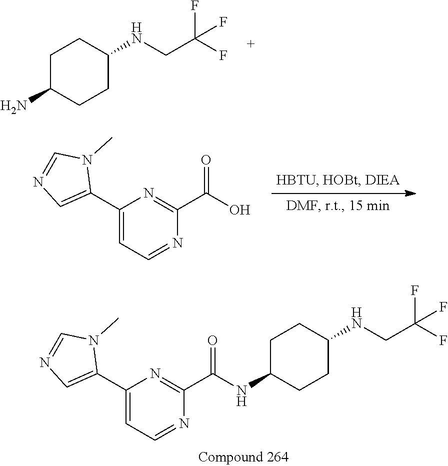

Step 3: Preparation of 4-(1-Methyl-1H-imidazol-5-yl)-N-((1r,4r)-4-((2,2,2-trifluoroethyl)amino)cyclohexyl)pyrimidine-2-carboxamide. Amide bond formation using the same procedure as Compound 256 to give 4-(1-methyl-1H-imidazol-5-yl)-N-((1r,4r)-4-((2,2,2-trifluoroethyl)amino)cyclohexyl)pyrimidine-2-carboxamide (31 mg, 0.081 mmol, 25%) as a white solid. LRMS (APCI) m/z 383.1 (M+H). ¹H NMR (400 MHz, Methanol-d₄) δ 8.84 (dd, J=5.4, 1.2 Hz, 1H), 7.97-7.84 (m, 3H), 4.19 (s, 3H), 3.99-3.85 (m, 1H), 3.32-3.23 (m, 2H), 2.61 (t, J=11.4 Hz, 1H), 2.08 (d, J=11.5 Hz, 4H), 1.52 (d, J=11.5 Hz, 2H), 1.30 (q, J=11.5 Hz, 2H).

### Example AG

### Synthesis of Compound 266 and Compound 270

### Preparation of 4-(1-methyl-1H-imidazol-5-yl)-N-((1s,3s)-3-(2-(trifluoromethyl)pyridin-4-yl)cyclobutyl)pyrimidine-2-carboxamide and 4-(1-methyl-1H-imidazol-5-yl)-N-((1r,3r)-3-(2-(trifluoromethyl)pyridin-4-yl)cyclobutyl)pyrimidine-2-carboxamide

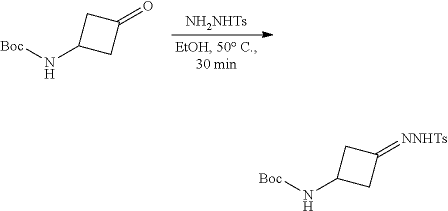

Step 1: Preparation of tert-Butyl (3-(2-tosylhydrazineylidene)cyclobutyl)carbamate: To a stirred solution of tert-butyl N-(3-oxocyclobutyl)carbamate (10 g, 54.0 mmol) in EtOH (100 mL) was added 4-toluenesulfonyl hydrazide (11.96 g, 64.25 mmol). The resulting mixture was stirred for 30 min at 50° C. and cooled to r.t. The resulting solid was filtered and washed with hexanes (100 mL) to provide tert-butyl (3-(2-tosylhydrazineylidene)cyclobutyl)carbamate 17.0 g, 48.1 mmol, 89%) as a white solid. LRMS (ES) m/z 298 (M+H).

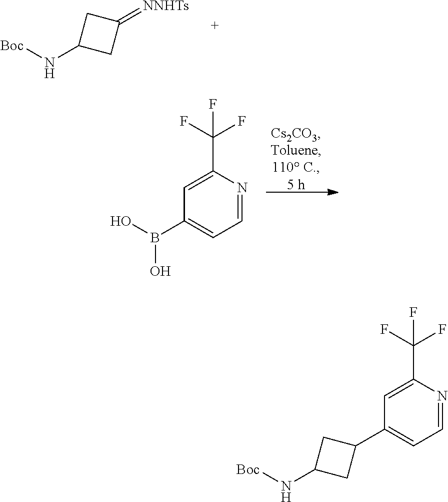

Step 2: Preparation of tert-Butyl (3-(2-(trifluoromethyl)pyridin-4-yl)cyclobutyl)carbamate. To a stirred solution of tert-butyl (3-(2-tosylhydrazineylidene)cyclobutyl)carbamate (9.2 g, 26.03 mmol) and (2-(trifluoromethyl)pyridin-4-yl)boronic acid (4.97 g, 26.03 mmol) in toluene (250 mL) was added Cs₂CO₃ (12.7 g, 39.04 mmol). The resulting mixture was stirred for 5 h at 110° C. It was cooled to r.t., water (100 mL) was added and the resulting mixture was extracted twice with EtOAc (100 mL). The organic layers were combined, washed with brine, dried over sodium sulfate, concentrated under reduced pressure and purified with C18 column chromatography, eluting with water (0.05% NH₄H₂O)/MeCN (2:3) to afford tert-butyl (3-(2-(trifluoromethyl)pyridin-4-yl)cyclobutyl)carbamate (2.0 g, 6.32 mmol, 2.91 mmol, 24%) as a yellow solid. LRMS (ES) m/z 261 (M+H-56).

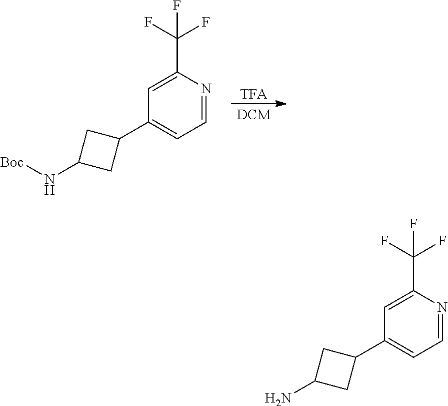 [SMILES: CC(C)(C)OC(=O)NC1CC(c2ccnc(C(F)(F)F)c2)C1 ; NC1CC(c2ccnc(C(F)(F)F)c2)C1 ; "TFA"]

Step 3: Preparation of 3-(2-(Trifluoromethyl)pyridin-4-yl)cyclobutan-1-amine: To tert-butyl (3-(2-(trifluoromethyl)pyridin-4-yl)cyclobutyl)carbamate (2.0 g, 6.32 mmol) was added DCM (20 mL) and TFA (5 mL). The resulting mixture was stirred at r.t. for 5 h, concentrated under reduced pressure and purified with C18 column chromatography, eluting with water (0.05% NH₃H₂O)/MeCN (10:1) to provide 3-(2-(trifluoromethyl)pyridin-4-yl)cyclobutan-1-amine (900 mg, 4.16 mmol, 66%) as an orange oil. LRMS (ES) m/z 217 (M+H).

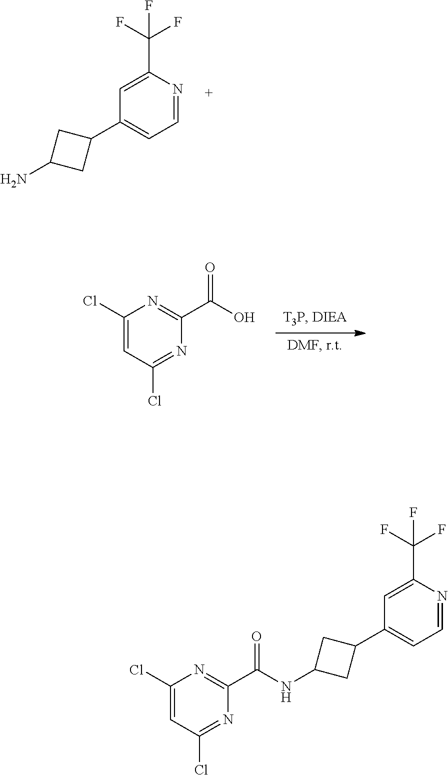 [SMILES: NC1CC(c2ccnc(C(F)(F)F)c2)C1 ; O=C(O)c1nc(Cl)cc(Cl)n1 ; "DMF, r.t." ; "T3P, DIEA" ; O=C(NC1CC(c2ccnc(C(F)(F)F)c2)C1)c1nc(Cl)cc(Cl)n1]

Step 4: Preparation of 4,6-Dichloro-N-(3-(2-(trifluoromethyl)pyridin-4-yl)cyclobutyl)pyrimidine-2-carboxamide: Prepared using same procedure as described for Compound 191 to give 4,6-dichloro-N-(3-(2-(trifluoromethyl)pyridin-4-yl)cyclobutyl)pyrimidine-2-carboxamide (150 mg, 0.38 mmol, 30%) as a yellow solid. LRMS (ES) m/z 391 (M+H).

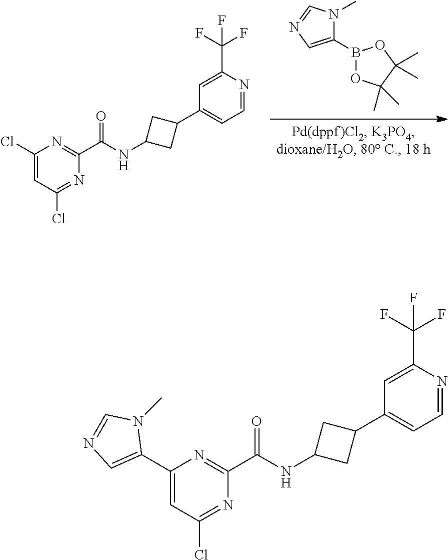

Step 5: Preparation of 4-Chloro-6-(1-methyl-1H-imidazol-5-yl)-N-(3-(2-(trifluoromethyl)pyridin-4-yl)cyclobutyl)pyrimidine-2-carboxamide. To a stirred solution of 4,6-dichloro-N-(3-(2-(trifluoromethyl)pyridin-4-yl)cyclobutyl)pyrimidine-2-carboxamide (140 mg, 0.36 mmol) and 1-methyl-5-(4,4,5,5-tetramethyl-1,3,2-dioxaborolan-2-yl)imidazole (60 mg, 0.29 mmol) in dioxane (2 mL) and H₂O (0.2 mL) were added Pd(dppf)Cl₂ (26 mg, 0.036 mmol) and K₃PO₄ (152 mg, 0.72 mmol). The resulting mixture was stirred for 3 h at 80° C. under a nitrogen atmosphere. The mixture was allowed to cool to room temperature and concentrated under reduced pressure to afford 4-chloro-6-(1-methyl-1H-imidazol-5-yl)-N-(3-(2-(trifluoromethyl)pyridin-4-yl)cyclobutyl)pyrimidine-2-carboxamide (180 mg, 0.41 mmol) of 4-chloro-6-(3-methylimidazol-4-yl)-N-{3-[2-(trifluoromethyl)pyridin-4-yl]cyclobutyl}pyrimidine-2-carboxamide (crude) as a brown oil which was used in the next step without purification. LRMS (ES) m/z 437 (M+H).

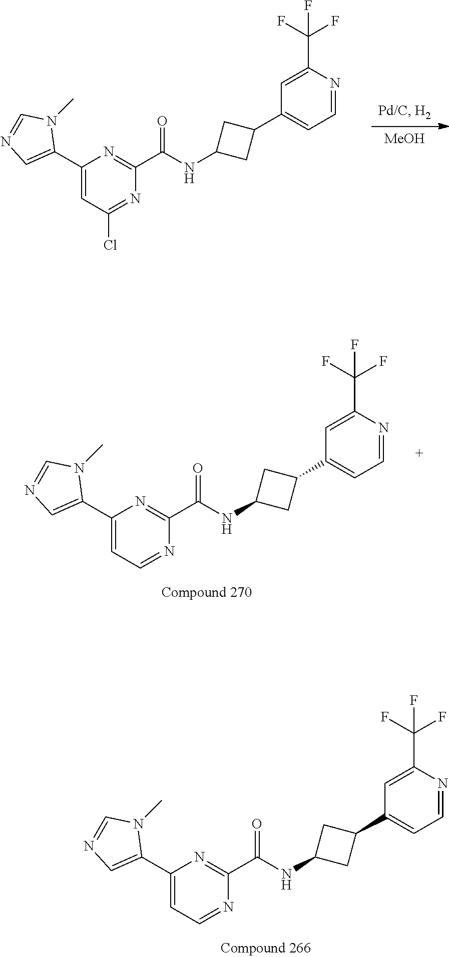 [SMILES: "MeOH" ; Cn1cncc1-c1cc(Cl)nc(C(=O)NC2CC(c3ccnc(C(F)(F)F)c3)C2)n1 ; "Pd/C, H2" ; Cn1cncc1-c1ccnc(C(=O)N[C@H]2C[C@H](c3ccnc(C(F)(F)F)c3)C2)n1 ; Cn1cncc1-c1ccnc(C(=O)N[C@H]2C[C@@H](c3ccnc(C(F)(F)F)c3)C2)n1]

Step 6: Preparation of 4-(1-Methyl-1H-imidazol-5-yl)-N-((1r,3r)-3-(2-(trifluoromethyl)pyridin-4-yl)cyclobutyl)pyrimidine-2-carboxamide and 4-(1-methyl-1H-imidazol-5-yl)-N-((1s,3s)-3-(2-(trifluoromethyl)pyridin-4-yl)cyclobutyl)pyrimidine-2-carboxamide 4-chloro-6-(1-methyl-1H-imidazol-5-yl)-N-(3-(2-(trifluoromethyl)pyridin-4-yl)cyclobutyl)pyrimidine-2-carboxamide (150 mg, 0.34 mmol) was combined with Pd/C (10%, 50% wet with water, 80 mg) and MeOH (2 mL). The resulting mixture was stirred under 30 psi H₂ for 7 h. It was filtered through celite, concentrated under reduced pressure and purified with C18 column chromatography eluting with water (0.05% NH₄HCO₃)/MeCN (3:2) followed by preparative HPLC with the following conditions: Column, CHIRAL ART Cellulose-SC, 2*25 cm, 5 um; mobile phase, Hex:DCM=3:1 (0.5% 2M NH₃-MeOH) and EtOH—(hold 50% EtOH-in 15 min) to provide 4-(1-methyl-1H-imidazol-5-yl)-N-((1r,3r)-3-(2-(trifluoromethyl)pyridin-4-yl)cyclobutyl)pyrimidine-2-carboxamide (8 mg, 0.020 mmol) as an off white solid and 4-(1-methyl-1H-imidazol-5-yl)-N-((1s,3s)-3-(2-(trifluoromethyl)pyridin-4-yl)cyclobutyl)pyrimidine-2-carboxamide (6 mg, 0.015 mmol) as an off white solid. 4-(1-methyl-1H-imidazol-5-yl)-N-((1r,3r)-3-(2-(trifluoromethyl)pyridin-4-yl)cyclobutyl)pyrimidine-2-carboxamide LRMS (ES) m/z 403 (M+H). ¹H NMR (400 MHz, Methanol-d₄) δ 8.85 (d, J=5.4 Hz, 1H), 8.66 (d, J=5.1 Hz, 1H), 7.95-7.88 (m, 3H), 7.81-7.76 (m, 1H), 7.68 (dd, J=5.2, 1.7 Hz, 1H), 4.78-4.66 (m, 1H), 4.20 (s, 3H), 3.84 (tt, J=10.1, 5.7 Hz, 1H), 2.85-2.51 (m, 4H). 4-(1-methyl-1H-imidazol-5-yl)-N-((1s,3s)-3-(2-(trifluoromethyl)pyridin-4-yl)cyclobutyl)pyrimidine-2-carboxamide: LRMS (ES) m/z 403 (M+H). ¹H NMR (400 MHz, Methanol-d₄) δ 8.83 (d, J=5.4 Hz, 1H), 8.62 (d, J=5.1 Hz, 1H), 7.93-7.86 (m, 3H), 7.83-7.78 (m, 1H), 7.62 (dd, J=5.1, 1.6 Hz, 1H), 4.72-4.59 (m, 1H), 4.18 (s, 3H), 3.46 (ddd, J=18.0, 10.3, 7.6 Hz, 1H), 2.95-2.84 (m, 2H), 2.49-2.36 (m, 2H).

Compounds 273 and 274 were prepared using the methods provided in the table below.

| Compound Number | Method of Preparation |
|---|---|
| 273 | Prepared using same procedure as Compound 266 |
| | and Compound 270 |
| 274 | Prepared using same procedure as Compound 266 |
| | and Compound 270 |

### Example AH

### Synthesis of Compound 275

### Preparation of N-((1r,4r)-4-(difluoromethoxy)cyclohexyl)-4-(1-methyl-1H-imidazol-5-yl)pyrimidine-2-carboxamide

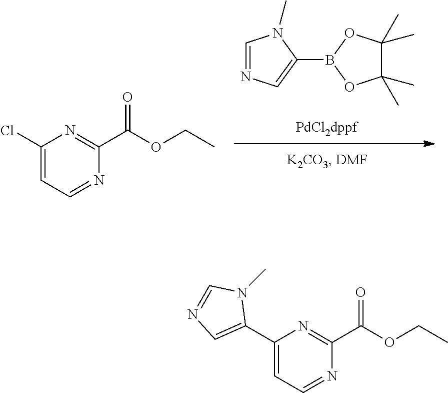 [SMILES: CCOC(=O)c1nccc(Cl)n1 ; Cn1cncc1B1OC(C)(C)C(C)(C)O1 ; CCOC(=O)c1nccc(-c2cncn2C)n1]

Step 1: Preparation of Ethyl 4-(1-methyl-1H-imidazol-5-yl)pyrimidine-2-carboxylate. To a solution of ethyl 4-chloropyrimidine-2-carboxylate (1.00 g, 5.36 mmol) in DMF (10 mL) was added potassium carbonate (1.49 g, 10.7 mmol), 1-methyl-5-(4,4,5,5-tetramethyl-1,3,2-dioxaborolan-2-yl)-1H-imidazole (1.22 g, 5.90 mmol), [1,1′-Bis(diphenylphosphino)ferrocene]dichloropalladium(II) (392 mg, 0.54 mmol), stirred in a sealed container at 130° C. in the oil bath for 1 h, cooled, filtered through Celite, concentrated, and directly purified by silica gel chromatography using 10% MeOH/DCM to yield ethyl 4-(1-methyl-1H-imidazol-5-yl)pyrimidine-2-carboxylate (1.21 g, 5.21 mmol, 97%) as a brown solid. The material was used in the next step without further purification. LRMS (APCI) m/z 233.1 (M+H).

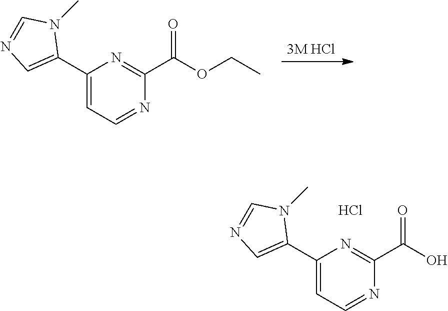 [SMILES: CCOC(=O)c1nccc(-c2cncn2C)n1 ; "3M HCl" ; Cn1cncc1-c1ccnc(C(=O)O)n1 ; Cl]

Step 2: Preparation of 4-(1-Methyl-1H-imidazol-5-yl)pyrimidine-2-carboxylic acid hydrochloride. A solution of ethyl 4-(1-methyl-1H-imidazol-5-yl)pyrimidine-2-carboxylate (1.21 g, 5.21 mmol) and 3 M aqueous hydrochloric acid (10 mL) was stirred at 100° C. for 2 h, cooled to r.t. and filtered. The filtrate was concentrated, sonicated in ether/hexanes and filtered to yield 4-(1-methyl-1H-imidazol-5-yl)pyrimidine-2-carboxylic acid hydrochloride (1.16 g, 4.84 mmol, 93%) as a brown solid. LRMS (APCI) m/z 205.0 (M+H).

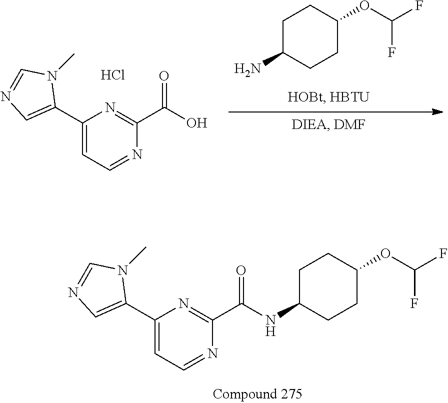

Step 3: Preparation of N-((1r,4r)-4-(difluoromethoxy)cyclohexyl)-4-(1-methyl-1H-imidazol-5-yl)pyrimidine-2-carboxamide. To a solution of 4-(1-methyl-1H-imidazol-5-yl)pyrimidine-2-carboxylic acid hydrochloride (100 mg, 0.42 mmol) and DIEA (0.29 mL, 1.66 mmol) in DMF (1 mL) was added HOBt (95.5 mg, 0.62 mmol), HBTU (236.4 mg, 0.62 mmol), and (1r,4r)-4-(difluoromethoxy)cyclohexan-1-amine (75.5 mg, 0.46 mmol). The reaction was capped, stirred overnight at r.t. for 17 h, purified by silica gel chromatography using a 0-10% MeOH/DCM gradient, filtered, and purified by reverse phase Prep HPLC (Phenomenex Gemini 5 micron C18 Axia pack 150×21.2 mm column) using a gradient of 3-40% MeCN/water with 0.1% formic acid to yield N-((1r,4r)-4-(difluoromethoxy)cyclohexyl)-4-(1-methyl-1H-imidazol-5-yl)pyrimidine-2-carboxamide (46 mg, 0.13 mmol, 32%) as a white solid. LRMS (ESI) m/z 352.1 (M+H). ¹H NMR (400 MHz, DMSO-d₆) δ 8.83 (s, 1H), 8.51 (d, J=8.2 Hz, 1H), 7.98-7.90 (m, 3H), 6.73 (t, J=76.8 Hz, 1H), 4.07 (s, 3H), 4.07-4.00 (m, 1H), 3.85-3.75 (m, 1H), 2.06-1.95 (m, 2H), 1.92-1.82 (m, 2H), 1.60-1.43 (m, 4H).

### Example AI

### Synthesis of Compound 276

### Preparation of N-(6-(difluoromethoxy)pyridin-3-yl)-4-(1-methyl-1H-imidazol-5-yl)pyrimidine-2-carboxamide

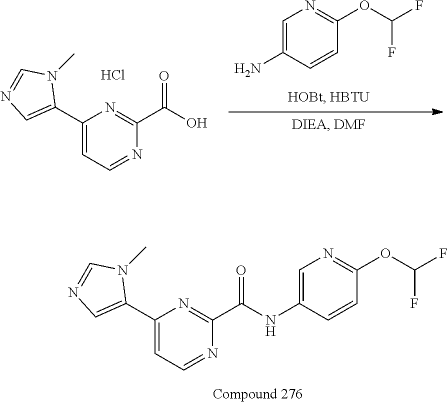

Preparation of N-(6-(difluoromethoxy)pyridin-3-yl)-4-(1-methyl-1H-imidazol yl)pyrimidine-2-carboxamide. To a solution of 4-(1-methyl-1H-imidazol-5-yl)pyrimidine carboxylic acid hydrochloride (99 mg, 0.41 mmol) and DIEA (0.29 mL, 1.65 mmol) in DMF (1 mL) was added 6-(difluoromethoxy)pyridin-3-amine (131.7 mg, 0.82 mmol), HOBt (94.5 mg, 0.62 mmol) and HBTU (234.0 mg, 0.62 mmol) and stirred at 70° C. for 3 h, diluted with water, and extracted with DCM. The combined organic layers were dried over sodium sulfate, concentrated, purified by silica gel chromatography using a 0-10% MeOH/DCM gradient, and purified by reverse phase prep HPLC (Phenomenex Gemini 5 micron C18 Axia pack 150×21.2 mm column) using a gradient of 3-40% water/acetonitrile with 0.1% formic acid to yield N-(6-(difluoromethoxy)pyridin-3-yl)-4-(1-methyl-1H-imidazol-5-yl)pyrimidine-2-carboxamide (24 mg, 0.07 mmol, 17%) as a white solid. LRMS (ESI) m/z 347.1 (M+H). ¹H NMR (400 MHz, DMSO-d₆) δ 10.95 (s, 1H), 8.93 (s, 1H), 8.75 (d, J=3.5 Hz, 1H), 8.38 (d, J=8.6 Hz 1H), 8.07-8.03 (m, 1H), 8.01 (s, 1H), 7.96 (s, 1H), 7.68 (t, J=73.2 Hz, 1H), 7.15 (d, J=9.3 Hz, 1H), 4.12 (s, 3H).

Compounds 278, 284, 294, 297, 299, 302, 305, 306, 310, 312, and 317 was prepared using the methods provided in the table below.

| Compound | |
|---|---|
| Number | Method of Preparation |
| 278 | Prepared in same fashion as Compound 275 |
| 284 | Prepared in same fashion as Compound 275 |
| 294 | Prepared in same fashion as Compound 275 |
| 297 | Prepared in same fashion as Compound 275 |
| 299 | Prepared in same fashion as Compound 275 |
| 302 | Prepared in same fashion as Compound 213 |
| 305 | Prepared in same fashion as Compound 213 |
| 306 | Beginning with 4-(1-methyl-1H-imidazol-5- |
| | yl)pyrimidine-2-carboxylic acid, amide bond |
| | formation as described for Compound 191. |
| 310 | Prepared in same fashion as Compound 275 |
| 312 | Prepared in same fashion as Compound 275 |
| 317 | Prepared in same fashion as Compound 213. |

### Example AJ

### Synthesis of Compound 319

### Preparation of N-(4-(3,3-difluorocyclobutoxy)phenyl)-4-(1-methyl-1H-imidazol-5-yl) pyrimidine-2-carboxamide

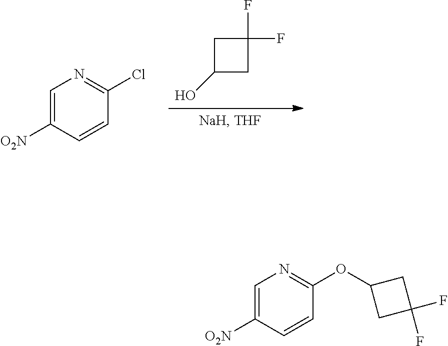

Step 1: Preparation of 2-(3,3-Difluorocyclobutoxy)-5-nitropyridine. To a solution of 2-chloro-5-nitropyridine (200 mg, 1.26 mmol) and 3,3-difluorocyclobutan-1-ol (150 mg, 1.39 mmol) in THF (2.5 mL) at 0° C. was added sodium hydride (101 mg, 2.52 mmol), stirred at r.t. for 30 min, diluted with water, and extracted with DCM. The combined organic layers were dried over sodium sulfate and concentrated to yield 2-(3,3-difluorocyclobutoxy)-5-nitropyridine (209 mg, 1.26 mmol, 72%). The product was used in the next step without further purification. LRMS (ESI) m/z 231.0 (M+H).

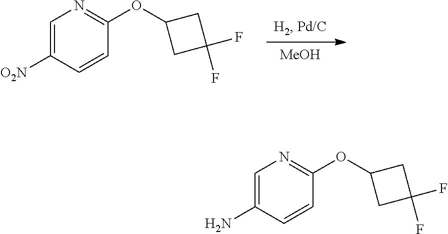 [SMILES: "MeOH" ; O=[N+]([O-])c1ccc(OC2CC(F)(F)C2)nc1 ; "H2, Pd/C" ; Nc1ccc(OC2CC(F)(F)C2)nc1]

Step 2: Preparation of 6-(3,3-Difluorocyclobutoxy)pyridin-3-amine. A solution of 2-(3,3-difluorocyclobutoxy)-5-nitropyridine (202 mg, 0.88 mmol), methanol (3 mL) and dioxane (1 mL) was purged with nitrogen for 5 min, added 5% Pd on activated charcoal (200 mg, 0.09 mmol), purged with nitrogen for 5 min and introduced to an atmosphere of hydrogen (balloon). The reaction was stirred at r.t. for 1.5 h, filtered through celite, and concentrated to yield 6-(3,3-difluorocyclobutoxy)pyridin-3-amine (175 mg, 0.87 mmol, 99.6%). The material was used in next step without further purification. LRMS (APCI) m/z 201.1 (M+H).

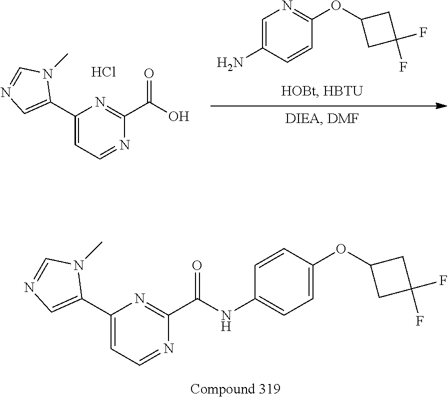

Step 3: Preparation of N-(4-(3,3-difluorocyclobutoxy)phenyl)-4-(1-methyl-1H-imidazol-5-yl)pyrimidine-2-carboxamide. Amide coupling step prepared in same fashion as Compound 276. LRMS (APCI) m/z 387.4 (M+H). ¹H NMR (400 MHz, DMSO-d₆) δ 10.81 (s, 1H), 9.05-9.00 (m, 1H), 8.63 (s, 1H), 8.48 (s, 1H), 8.26 (s, 1H), 8.21 (d, J=9.4 Hz, 1H), 8.12-8.07 (m, 1H), 6.93 (d, J=9.0 Hz, 1H), 5.10 (s, 1H), 4.19 (s, 3H), 3.5 (s, 1H), 3.22-3.09 (m, 2H), 2.80-2.63 (m, 2H).

Compounds 320, 330, 343, and 344 were prepared using the methods provided in the table below.

| Compound | |
|---|---|
| Number | Method of Preparation |
| 320 | Prepared in same fashion as Compound 275 |
| 330 | Prepared in the same fashion as Compound 319 |
| 343 | Prepared in same fashion as Compound 275 |
| 344 | Beginning with (1S,3S)-3-(2-methoxyethoxy)cyclopentan- |
| | 1-amine, synthesized using same procedure as Compound |
| | 351 |

### Example AK

### Synthesis of Compound 347

### Preparation of 4-(difluoromethyl)-N-((1r,4r)-4-hydroxycyclohexyl)-6-(1-methyl-1H-imidazol-5-yl)pyrimidine-2-carboxamide

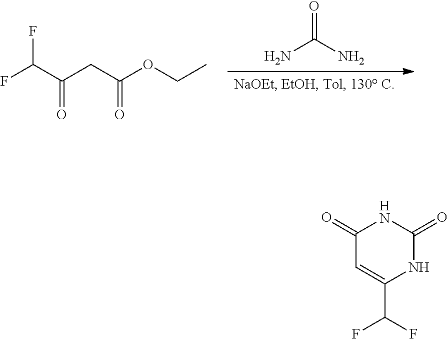

Step 1: Preparation of 6-(Difluoromethyl)pyrimidine-2,4(1H,3H)-dione. To a stirred solution of ethyl 4,4-difluoro-3-oxobutanoate (8.6 g, 51.8 mmol) and urea (3.73 g, 62.1 mmol) in toluene (100 mL) was added NaOEt (35.23 g, 103.538 mmol, 2 equiv, 20% in EtOH). The resulting mixture was stirred at r.t. for 30 min followed by 130° C. for 24 h. The mixture was cooled to r.t. and concentrated under reduced pressure to give 6-(difluoromethyl)pyrimidine-2,4(1H,3H)-dione (12.0 g) as a brown solid which was used in the subsequent step without further purification. LRMS (ES) m/z 163 (M+H).

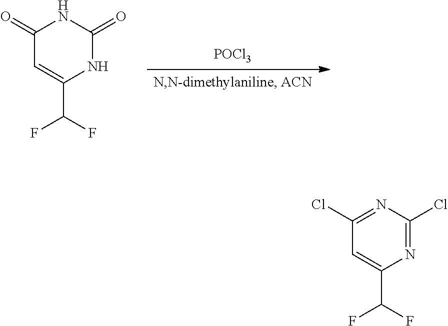 [SMILES: O=c1cc(C(F)F)[nH]c(=O)[nH]1 ; "POCl3" ; "N,N-dimethylaniline, ACN" ; FC(F)c1cc(Cl)nc(Cl)n1]

Step 2: Preparation of 2,4-Dichloro-6-(difluoromethyl)pyrimidine. To a stirred solution of 6-(difluoromethyl)pyrimidine-2,4(1H,3H)-dione (12.0 g, 74.0 mmol) and N,N-dimethylaniline (9.0 g, 74.0 mmol) in ACN (120 mL) at 0° C. was added phosphorus oxychloride (45.4 g, 296.1 mmol) dropwise over a period of 15 min. The resulting mixture was stirred at 95° C. overnight. It was cooled to r.t., carefully quenched with water (100 mL) at 0° C., extracted twice with DCM (100 mL), washed with brine, dried over sodium sulfate, concentrated under reduced pressure and purified with silica gel using 5% ethyl acetate/petroleum ether to afford 2,4-dichloro-6-(difluoromethyl)pyrimidine (4.0 g, 20.2 mmol, 27%) as a light yellow oil. (observed low boiling point with no LC/MS signal). ¹H NMR (300 MHz, Methanol-d₄) δ 7.87 (s, 1H), 6.72 (t, J=54.0 Hz, 1H).

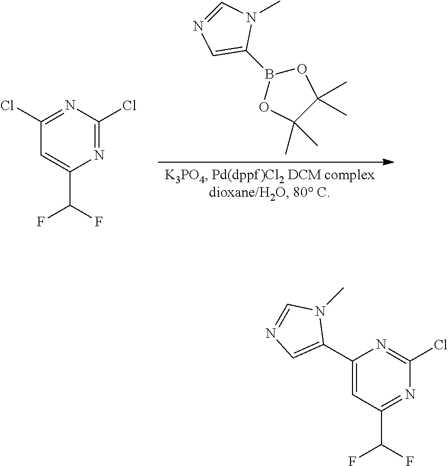

Step 3: Preparation of 2-Chloro-4-(difluoromethyl)-6-(1-methyl-1H-imidazol-5-yl)pyrimidine. To a stirred solution of 2,4-dichloro-6-(difluoromethyl)pyrimidine (1.15 g, 5.78 mmol) and 1-methyl-5-(4,4,5,5-tetramethyl-1,3,2-dioxaborolan-2-yl)imidazole (1.20 g, 5.78 mmol) in 1,4-dioxane (15 mL) and H₂O (1.5 mL) were added Pd(dppf)Cl₂.CH₂Cl₂ (471 mg, 0.578 mmol) and K₃PO₄ (2.45 g, 11.56 mmol). The resulting mixture was stirred at 80° C. overnight under a nitrogen atmosphere. The mixture was cooled to r.t., filtered to remove solids, concentrated under reduced pressure and purified with silica gel using 10% MeOH/DCM to afford 2-chloro-4-(difluoromethyl)-6-(1-methyl-1H-imidazol-5-yl)pyrimidine (900 mg, 3.69 mmol, 64%) as a brown oil. LRMS (ES) m/z 245 (M+H).

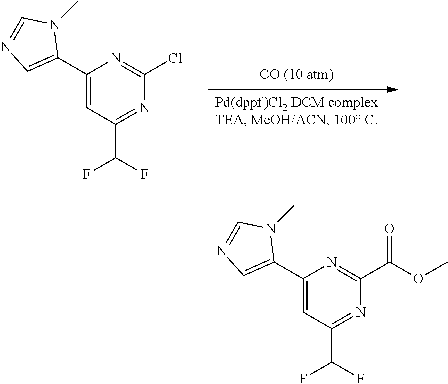 [SMILES: Cn1cncc1-c1cc(C(F)F)nc(Cl)n1 ; "CO (10 atm)" ; "Pd(dppf)Cl2 DCM complexTEA, MeOH/ACN, 100° C." ; COC(=O)c1nc(-c2cncn2C)cc(C(F)F)n1]

Step 4: Preparation of Methyl 4-(difluoromethyl)-6-(1-methyl-1H-imidazol-5-yl)pyrimidine-2-carboxylate. To a solution of 2-chloro-4-(difluoromethyl)-6-(1-methyl-1H-imidazol-5-yl)pyrimidine (900 mg, 3.69 mmol) in MeOH (8 mL) and ACN (2 mL) were added Pd(dppf)Cl₂.CH₂Cl₂ (603 mg, 0.74 mmol, 0.2 equiv) and TEA (747 mg, 7.4 mmol, 2 equiv) in a pressure reactor. The mixture was purged with nitrogen for 1 min and then pressurized to 10 atm with carbon monoxide and stirred at 100° C. overnight. The mixture was allowed to cool to r.t., concentrated under reduced pressure and purified by C18 column chromatography eluting with water (0.05% NH₄HCO₃)/MeCN (2:1) to provide methyl 4-(difluoromethyl)-6-(1-methyl-1H-imidazol-5-yl)pyrimidine-2-carboxylate (440 mg, 1.64 mmol, 45%) as an orange solid. LRMS (ES) m/z 269 (M+H).

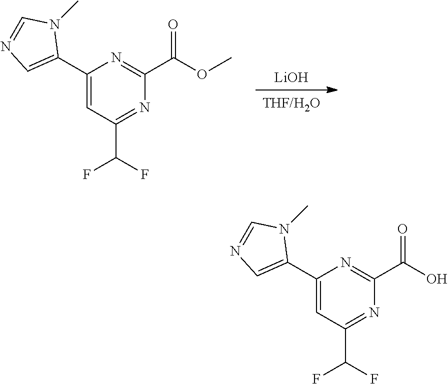 [SMILES: COC(=O)c1nc(-c2cncn2C)cc(C(F)F)n1 ; "LiOH" ; "THF/H2O" ; Cn1cncc1-c1cc(C(F)F)nc(C(=O)O)n1]

Step 5: Preparation of 4-(Difluoromethyl)-6-(1-methyl-1H-imidazol-5-yl)pyrimidine-2-carboxylic acid. To a stirred solution of methyl 4-(difluoromethyl)-6-(1-methyl-1H-imidazol-5-yl)pyrimidine-2-carboxylate (430 mg, 1.60 mmol) in THF (8 mL) and H₂O (1 mL) was added lithium hydroxide (96 mg, 2.41 mmol, 1.50). The resulting mixture was stirred at r.t. for 1 h, the pH was adjusted to 6-7 using concentrated HCl and the resulting mixture was concentrated under reduced pressure to afford crude 4-(difluoromethyl) (1-methyl-1H-imidazol-5-yl)pyrimidine-2-carboxylic acid (400 mg, 1.57 mmol) as a yellow solid which was used in subsequent steps without additional purification. LRMS (ES) m/z 255 (M+H).

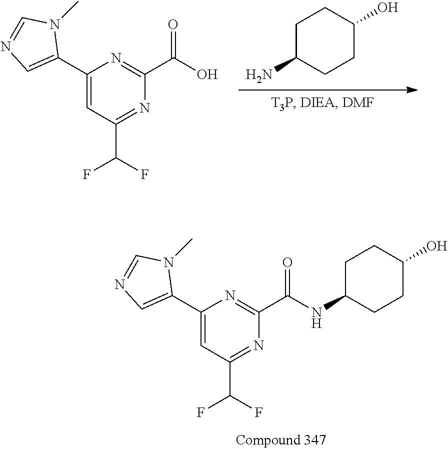

Step 6: Preparation of 4-(Difluoromethyl)-N-((1r,4r)-4-hydroxycyclohexyl)-6-(1-methyl-1H-imidazol-5-yl)pyrimidine-2-carboxamide Prepared using same amide bound formation conditions as described for Compound 191 to provide 4-(difluoromethyl)-N-((1r,4r)-4-hydroxycyclohexyl)-6-(1-methyl-1H-imidazol-5-yl)pyrimidine-2-carboxamide (24 mg, 0.068 mmol, 16%) as an off-white solid. LRMS (ES) m/z 352 (M+H). ¹H NMR (300 MHz, DMSO-d6) δ 8.42 (d, J=8.2 Hz, 1H), 8.24-8.12 (m, 2H), 7.98 (s, 1H), 7.03 (t, J=54.1 Hz, 1H), 4.59 (d, J=4.3 Hz, 1H), 4.08 (s, 3H), 3.79-3.69 (m, 1H), 3.58-3.38 (m, 1H), 1.85 (d, J=10.1 Hz, 4H), 1.44 (q, J=11.6 Hz, 2H), 1.27 (q, J=11.1 Hz, 2H).

Compound 350 was prepared using the methods provided in the table below.

| Compound | |
|---|---|
| Number | Method of Preparation |
| 350 | Prepared in same fashion as Compound 347 |

### Example AL

### Synthesis of Compound 351

### Preparation of N-((1R,3R)-3-(2-methoxyethoxy)cyclopentyl)-4-(1-methyl-1H-imidazol-5-yl)pyrimidine-2-carboxamide

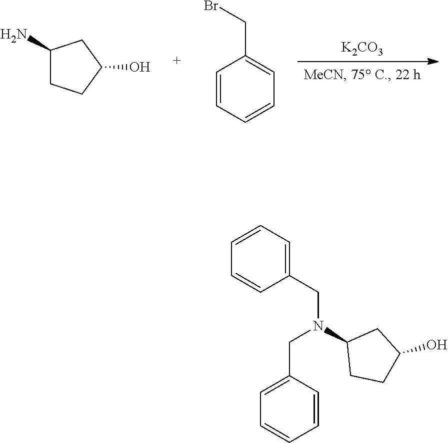

Step 1: Preparation of (1R,3R)-3-(Dibenzylamino)cyclopentan-1-ol: (1R,3R)-3-aminocyclopentan-1-ol hydrochloride (1.75 g, 12.7 mmol) and potassium carbonate (1.76 g, 12.72 mmol) were dissolved in acetonitrile (25 mL). Benzyl bromide (4.57 g, 26.71 mmol) was added and the mixture was heated at 75° C. for 22 h with a condenser attached. The reaction was cooled to r.t., filtered through celite and concentrated under reduced pressure. The product was purified with silica gel using 5% MeOH/DCM to provide (1R,3R)-3-(dibenzylamino)cyclopentan-1-ol (2.9 g, 10.31 mmol, 81%) which was used in the subsequent step without further purification. LRMS (APCI) m/z 282.1 (M+H).

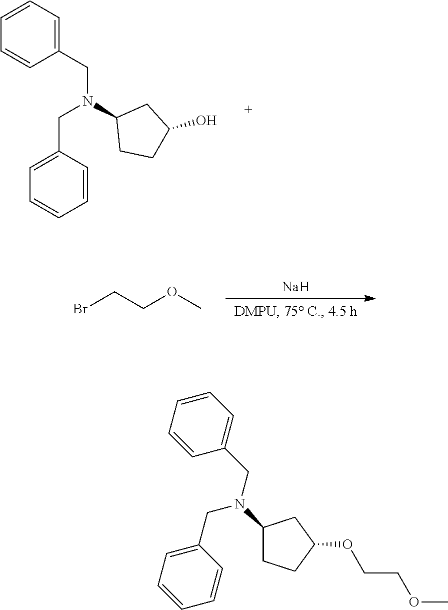

Step 2: Preparation of (1R,3R)-N,N-dibenzyl-3-(2-methoxyethoxy)cyclopentan-1-amine: (1R,3R)-3-(dibenzylamino)cyclopentan-1-ol (2.7 g, 9.595 mmol) was dissolved in N, N′-dimethylpropyleneurea (20 mL), placed under nitrogen, and cooled to 0° C. with an ice bath. Sodium hydride (60% suspension in mineral oil) (0.65 g, 16.3 mmol) was added portionwise and the resulting mixture was stirred at 0° C. for 10 min. 2-bromoethyl methyl ether (1.0 mL, 10.6 mmol) was added portionwise and the ice bath was removed. The reaction was stirred for 15 min, during which time it was allowed to warm to r.t. The mixture was then heated in an oil bath at 75° C. for 2 h. Additional sodium hydride (0.384 g, 9.60 mmol) and 2-bromoethyl methyl ether (0.914 mL, 9.60 mmol) were added and the reaction was stirred at 75° C. for 1 h. Additional sodium hydride (0.384 g, 9.60 mmol) and 2-bromoethyl methyl ether (0.914 mL, 9.60 mmol) were added and the reaction was stirred at 75° C. for 1 additional h. The reaction was then cooled to 0° C. with an ice bath and water was added dropwise. The resulting mixture was extracted with EtOAc (175 mL). The organic layer was washed with saturated aqueous ammonium chloride and brine. It was dried over sodium sulfate and concentrated under reduced pressure. The product was purified with silica gel using 40% EtOAc/hexanes. The unreacted starting material was recovered, re-reacted in the same manner, and purified in the same manner. The products were combined to yield (1R,3R)-N,N-dibenzyl-3-(2-methoxyethoxy)cyclopentan-1-amine (1.7 g, 5.01 mmol, 52%) as a viscous colorless oil. LRMS (APCI) m/z=340.1 (M+H).

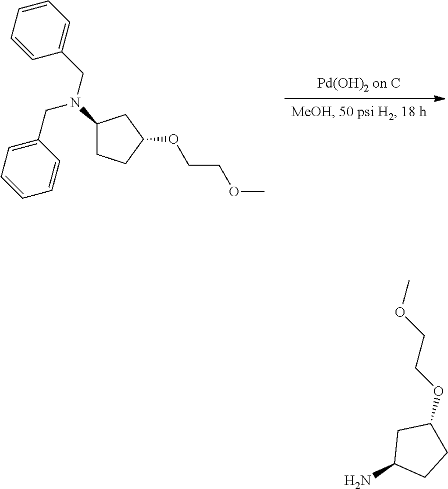 [SMILES: COCCO[C@@H]1CC[C@@H](N(Cc2ccccc2)Cc2ccccc2)C1 ; "MeOH, 50 psi H2, 18 h" ; "Pd(OH)2 on C" ; COCCO[C@@H]1CC[C@@H](N)C1]

Step 3: Preparation of (1R,3R)-3-(2-Methoxyethoxy)cyclopentan-1-amine: (1R,3R)-N,N-dibenzyl-3-(2-methoxyethoxy)cyclopentan-1-amine (1.7 g, 5.01 mmol) was dissolved in methanol (15 mL). Palladium hydroxide on carbon (20%) (0.703 g, 1.00 mmol) was added and the mixture was stirred at r.t. under 50 psi hydrogen gas for 18 h. The reaction mixture was filtered through celite and the filtrate was concentrated under reduced pressure to yield (1R,3R)-3-(2-methoxyethoxy)cyclopentan-1-amine as a colorless gelatinous solid. LRMS (APCI) m/z=160.6 (M+H).

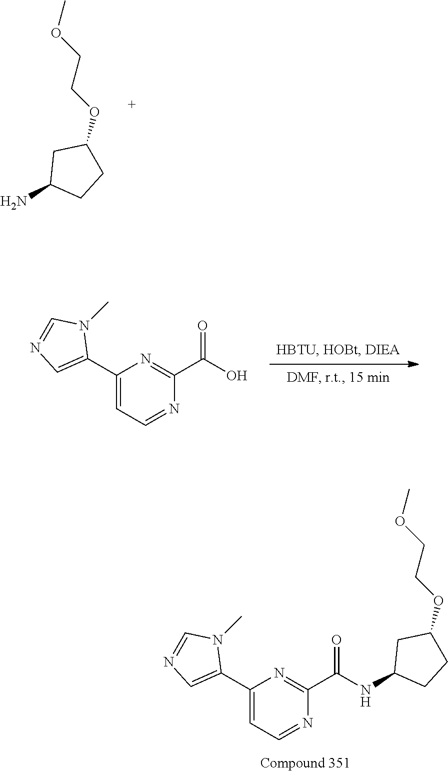 [SMILES: COCCO[C@@H]1CC[C@@H](N)C1 ; Cn1cncc1-c1ccnc(C(=O)O)n1 ; "DMF, r.t., 15 min" ; "HBTU, HOBt, DIEA" ; COCCO[C@@H]1CC[C@@H](NC(=O)c2nccc(-c3cncn3C)n2)C1]

Step 4: Preparation of (N-[(1R,3R)-3-(2-methoxyethoxy)cyclopentyl]-4-(3-methylimidazol-4-yl)pyrimidine-2-carboxamide). Amide coupling performed in the same fashion as Compound 256.

Compound 356 was prepared using the methods provided in the table below.

| Compound | |
|---|---|
| Number | Method of Preparation |
| 356 | Prepared in same fashion as Compound 276 |

Step 1: Preparation of 4-chloro-N-(6-(difluoromethyl)pyridin-3-yl)-6-methoxypyrimidine-2-carboxamide: Prepared with 4,6-dichloropyrimidine-2-carboxylic acid, amide bond formation as described for Compound 191 followed by nucleophilic aromatic substitution with sodium methoxide as described for Compound 192.

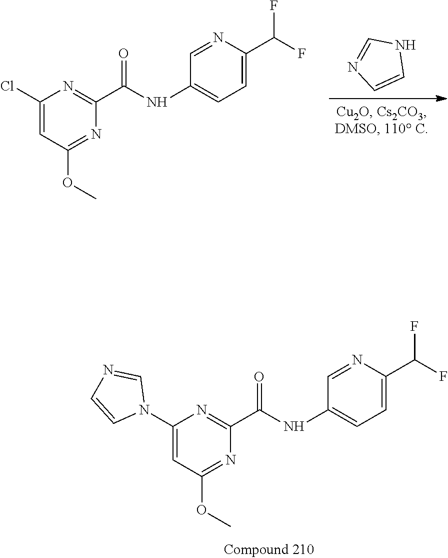

Step 2: Preparation of N-(6-(difluoromethyl)pyridin-3-yl)-4-(1H-imidazol-1-yl)-6-methoxypyrimidine-2-carboxamide. To a stirred solution of 4-chloro-N-(6-(difluoromethyl)pyridin-3-yl)-6-methoxypyrimidine-2-carboxamide (200 mg, 0.35, 55% purity) and imidazole (29 mg, 0.42 mmol) in DMSO (3 mL) were added Cs₂CO₃ (228 mg, 0.700 mmol) and Cu₂O (10 mg, 0.070 mmol). The resulting mixture was stirred at 110° C. for 2 h under a nitrogen atmosphere. It was cooled to r.t., filtered to remove solids, and purified by C18 column chromatography eluting with water (0.05% NH₄HCO₃)/MeCN (2:1) followed by reverse phase HPLC using the following conditions (SHIMADZU HPLC): Column, Xselect CSH C18 OBD Column 30*150 mm, 5 um; mobile phase, Water (0.1% FA) and ACN (5% ACN up to 25% in 8 min) to give N-(6-(difluoromethyl)pyridin-3-yl)-4-(1H-imidazol-1-yl)-6-methoxypyrimidine-2-carboxamide (25 mg, 0.072 mmol, 20%) as a white solid. LRMS (ES) m/z 347 (M+H). ¹H NMR (400 MHz, DMSO-d6) δ 10.96 (s, 1H), 9.13 (d, J=2.4 Hz, 1H), 8.98 (s, 1H), 8.47 (dd, J=8.4, 2.4 Hz, 1H), 8.28 (t, J=1.4 Hz, 1H), 7.79 (d, J=8.6 Hz, 1H), 7.58 (s, 1H), 7.22 (s, 1H), 6.97 (t, J=55.1 Hz, 1H), 4.12 (s, 3H).

Compounds 205, 215, and 354 were prepared using the methods provided in the table below.

| Compound | |
|---|---|
| Number | Method of Preparation |
| 205 | Beginning with 4,6-dichloropyrimidine-2-carboxylic acid, amide bond |
| | formation as described for Compound 191 followed by Stille coupling using |
| | tributyl(cyclopropyl)stannane using the same procedure as Compound 202, |
| | followed by copper coupling as described for Compound 210. |
| 215 | Beginning with 4,6-dichloropyrimidine-2-carboxylic acid, amide bond |
| | formation as described for Compound 191followed by Stille coupling as |
| | described for Compound 202 and copper coupling with imidazole using the |
| | same procedure described for Compound 210. |
| 354 | Beginning with 4-chloropyrimidine-2-carboxylic acid, acyl chloride |
| | preparation and amide bond formation using same procedure as Compound |
| | 189, followed by nucleophilic aromatic substitution with imidazole using same |
| | procedure as described for Compound 36. |

### Example AN

### Synthesis of Compound 249

### Preparation of N-(6-(2-hydroxypropan-2-yl)pyridin-3-yl)-4-(1H-imidazol-1-yl)pyrimidine-2-carboxamide

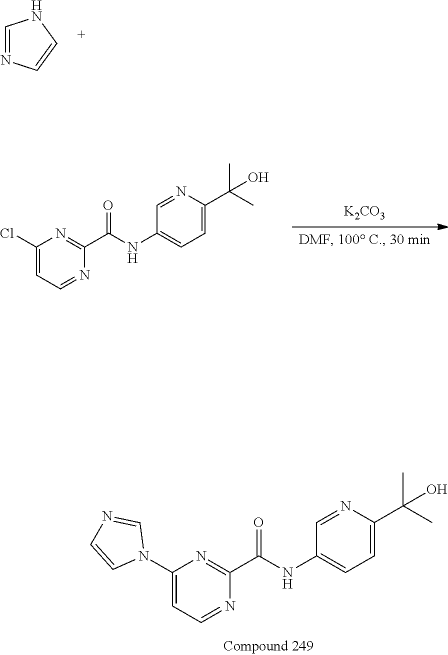 [SMILES: c1c[nH]cn1 ; CC(C)(O)c1ccc(NC(=O)c2nccc(Cl)n2)cn1 ; "K2CO3" ; "DMF, 100° C., 30 min" ; CC(C)(O)c1ccc(NC(=O)c2nccc(-n3ccnc3)n2)cn1]

Preparation of N-(6-(2-hydroxypropan-2-yl)pyridin-3-yl)-4-(1H-imidazol-1-yl)pyrimidine-2-carboxamide. Prepared using the same procedure as described for Compound 36 and purified by reverse phase prep HPLC (Phenomenex Gemini 5 micron C18 Axia pack 150×21.2 mm column) using a gradient of 3-40% water/acetonitrile with 0.1% formic acid to yield to provide N-(6-(2-hydroxypropan-2-yl)pyridin-3-yl)-4-(1H-imidazol-1-yl)pyrimidine-2-carboxamide (22 mg, 0.068 mmol, 48%) as a white solid. LRMS (APCI) m/z 325.1 (M+H). ¹H NMR (400 MHz, Methanol-d₄) δ 9.08 (d, J=5.6 Hz, 1H), 9.03 (s, 1H), 8.98 (d, J=2.5 Hz, 1H), 8.33 (dd, J=8.7, 2.5 Hz, 1H), 8.24 (s, 1H), 8.00 (d, J=5.6 Hz, 1H), 7.75 (d, J=8.7 Hz, 1H), 7.26 (s, 1H), 1.59 (s, 6H).

Compounds 207, 211, 216, 217, 236-239, 258, 261, 265, 268, and 293 were prepared using the methods provided in the table below.

| Compound | |
|---|---|
| Number | Method of Preparation |
| 207 | Beginning with 4,6-dichloropyrimidine-2-carboxylic acid, amide bond |
| | formation as described for Compound 191 followed by Stille coupling using |
| | tetramethyl stannane with same procedure as Compound 202, followed by |
| | copper coupling as described for Compound 210. |
| 211 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 216 | Beginning with 4,6-dichloropyrimidine-2-carboxylic acid, amide bond |
| | formation as described for Compound 191 followed by copper coupling as |
| | described for Compound 210 |
| 217 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 236 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 237 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 238 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 239 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 258 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 261 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36 |
| 265 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 268 | Beginning with 4-(1H-imidazol-1-yl)pyrimidine-2-carboxylic acid and 3-(2- |
| | (trifluoromethyl)pyridin-4-yl)cyclobutan-1-amine, prepared using same amide |
| | bond formation conditions as Compound 191 |
| 293 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |

### Example AO

### Synthesis of Compound 346

### Preparation of 4-(difluoromethyl)-6-(1H-imidazol-1-yl)-N-((1r,4r)-4-methoxycyclohexyl)pyrimidine-2-carboxamide

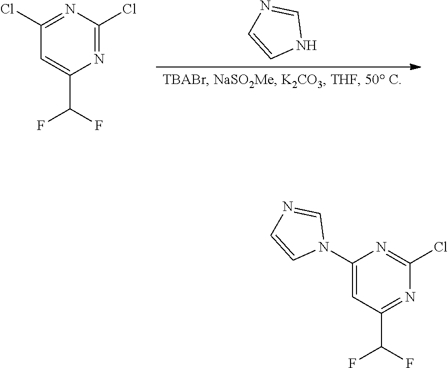

Step 1: Preparation of 2-Chloro-4-(difluoromethyl)-6-(1H-imidazol-1-yl)pyrimidine: To a stirred solution of 2,4-dichloro-6-(difluoromethyl)pyrimidine (1.0 g, 5.0 mmol) and imidazole (339 mg, 5.0 mmol) in THF (10 mL) were added TBAB (162 mg, 0.50 mmol), NaSO₂Me (15 mg, 0.15 mmol) and K₂CO₃ (1.39 g, 10.0 mmol). The resulting mixture was stirred at 50° C. overnight. The mixture was cooled to r.t., filtered to remove solids and purified with silica gel column chromatography using 10% MeOH/DCM to afford 2-chloro-4-(difluoromethyl)-6-(1H-imidazol-1-yl)pyrimidine (600 mg, 2.61 mmol, 52%) as a yellow solid. LRMS (ES) m/z 231 (M+H).

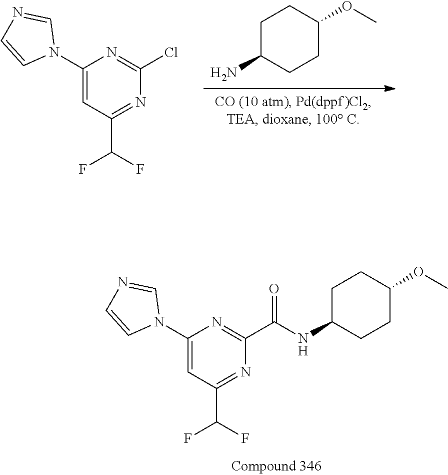

Step 2: Preparation of 4-(Difluoromethyl)-6-(1H-imidazol-1-yl)-N-(1r,4r)-4-methoxycyclohexyl)pyrimidine-2-carboxamide. To a stirred solution 2-chloro-4-(difluoromethyl)-6-(1H-imidazol-1-yl)pyrimidine (100 mg, 0.43 mmol, 1 equiv) and (1r,4r)-4-methoxycyclohexan-1-amine hydrochloride (180 mg, 1.1 mmol, 2.5 equiv) in dioxane (10 mL) were added Pd(dppf)Cl₂CH₂Cl₂ (35 mg, 0.043 mmol, 0.1 equiv) and TEA (218 mg, 2.15 mmol, 5 equiv). The resulting mixture was purged with nitrogen for 1 min and then pressurized to 10 atm with carbon monoxide and stirred at 100° C. for 18 h. The mixture was cooled to r.t., concentrated under reduced pressure and purified by C18 column chromatography eluting with water (0.05% NH₄HCO₃)/MeCN (2:1) followed by preparative HPLC with the following conditions (SHIMADZU HPLC) Column, XSelect CSH Fluoro Phenyl, 30*150 mm, 5 μm; mobile phase, water (10 mmol/L NH₄HCO₃) and ACN (13% ACN up to 48% in 7 min) to give 4-(difluoromethyl)-6-(1H-imidazol-1-yl)-N-((1r,4r)-4-methoxycyclohexyl)pyrimidine-2-carboxamide (50 mg, 0.14 mmol, 32%) as an off-white solid. LRMS (ES) m/z 352 (M+H). 1H NMR (300 MHz, DMSO-d6) δ 9.07 (t, J=1.1 Hz, 1H), 8.77 (d, J=8.5 Hz, 1H), 8.42-8.32 (m, 2H), 7.31-6.84 (m, 2H), 3.96-3.65 (m, 1H), 3.26 (s, 3H), 3.20-3.06 (m, 1H), 2.06 (d, J=12.2 Hz, 2H), 1.86 (d, J=12.4 Hz, 2H), 1.63-1.45 (m, 2H), 1.33-1.15 (m, 2H).

Compounds 204, 247, 251, 257, 269, 308, and 349 were prepared using the methods provided in the table below.

| Compound | |
|---|---|
| Number | Method of Preparation |
| 204 | Beginning with 4,6-dichloropyrimidine-2-carboxylic acid, amide bond |
| | formation as described for Compound 191 followed by Stille coupling using |
| | tributyl(cyclopropyl)stannane using the same procedure as Compound 202, |
| | followed by copper coupling as described for Compound 210 |
| 247 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 251 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 257 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 269 | Beginning with 4-(1H-imidazol-1-yl)pyrimidine-2-carboxylic acid and 3-(6- |
| | (trifluoromethyl)pyridin-3-yl)cyclobutan-1-amine, amide bond formation as |
| | described for Compound 191. |
| 308 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 349 | Prepared in same fashion as Compound 346 |

### Example AP

### Synthesis of Compound 263

### Preparation of 4-(1H-imidazol-1-yl)-N-(1-phenylpiperidin-4-yl)pyrimidine-2-carboxamide

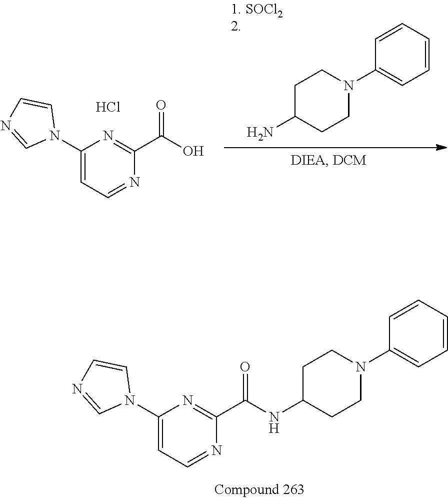

Preparation of 4-(1H-imidazol-1-yl)-N-(1-phenylpiperidin-4-yl)pyrimidine-2-carboxamide. A suspension of 4-(1H-imidazol-1-yl)pyrimidine-2-carboxylic acid hydrochloride (150 mg, 0.66 mmol) in neat thionyl chloride (2 mL) was stirred at 80° C. in a sealed tube for 2 h with venting as needed, cooled, concentrated, added DCM, cooled to 0 C, added 1-phenylpiperidin-4-amine (233 mg, 1.32 mmol), DIEA (0.58 mL, 3.32 mmol), stirred at r.t. for 1 h, concentrated, filtered, and purified by reverse phase prep HPLC (Phenomenex Gemini 5 micron C18 Axia pack 150×21.2 mm column) using a gradient of 3-40% water/acetonitrile with 0.1% formic acid to yield 4-(1H-imidazol-1-yl)-N-(1-phenylpiperidin-4-yl)pyrimidine-2-carboxamide (7.9 mg, 0.02 mmol, 3%). LRMS (ESI) m/z 349.2 (M+H). ¹H NMR (400 MHz, DMSO-d₆) δ 9.05 (d, J=5.6 Hz, 1H), 8.93 (s, 1H), 8.85 (d, J=8.3 Hz, 1H), 8.24 (s, 1H), 8.06 (d, J=5.7 Hz, 1H), 7.24-7.18 (m, 3H), 6.97 (d, J=8.1 Hz, 2H), 6.75 (t, J=7.2 Hz, 1H), 4.09-3.98 (m, 1H), 3.77 (d, J=12.6 Hz, 2H), 2.82 (td, J=11.3, 2.7 Hz, 2H), 1.91-1.74 (m, 4H).

Compounds 212, 219, 260, 301, and 313 were prepared using the methods provided in the table below.

| Compound | |
|---|---|
| Number | Method of Preparation |
| 212 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 219 | Prepared in same fashion as Compound 220, except last step nucleophilic |
| | aromatic substitution with imidazole using same procedure as described |
| | Compound 36. |
| 260 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 301 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |
| 313 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |

### Example AQ

### Synthesis of Compound 208

### Preparation of 4-(1H-imidazol-1-yl)-6-methoxy-N-((1r,4r)-4-methoxycyclohexyl)pyrimidine-2-carboxamide

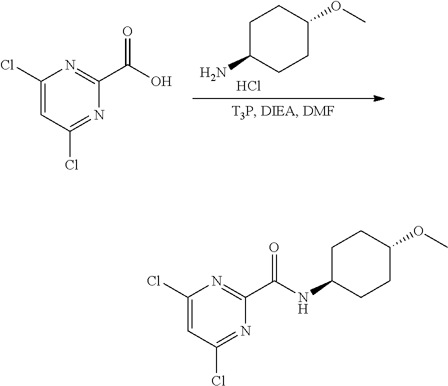

Step 1: Preparation of 4,6-Dichloro-N-((1r,4r)-4-methoxycyclohexyl)pyrimidine-2-carboxamide: Prepared using same amide bond coupling conditions described for Compound 191 to give 4,6-dichloro-N-((1r,4r)-4-methoxycyclohexyl)pyrimidine-2-carboxamide (550 mg, 1.81 mmol, 71%) as a white solid. LRMS (ES) m/z 304 (M+H).

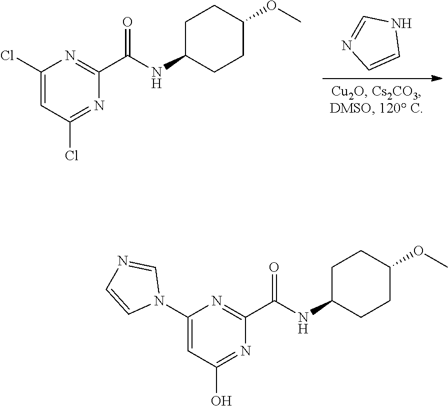

Step 2: Preparation of 4-Hydroxy-6-(1H-imidazol-1-yl)-N-((1r,4r)-4-methoxycyclohexyl)pyrimidine-2-carboxamide. Prepared using the same copper coupling conditions as Compound 210 to provide 4-hydroxy-6-(1H-imidazol-1-yl)-N-((1r,4r)-4-methoxycyclohexyl)pyrimidine-2-carboxamide which was used in the next step without additional purification. LRMS (ES) m/z 318 (M+H).

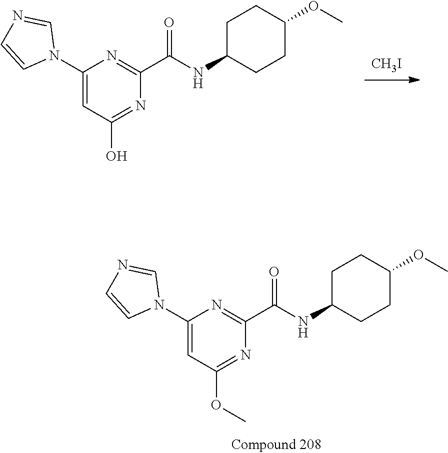 [SMILES: CO[C@H]1CC[C@H](NC(=O)c2nc(O)cc(-n3ccnc3)n2)CC1 ; "CH3I" ; COc1cc(-n2ccnc2)nc(C(=O)N[C@H]2CC[C@H](OC)CC2)n1]

Step 3: Preparation of 4-(1H-Imidazol-1-yl)-6-methoxy-N-((1r,4r)-4-methoxycyclohexyl)pyrimidine-2-carboxamide. To crude 4-hydroxy-6-(1H-imidazol-1-yl)-N-((1r,4r)-4-methoxycyclohexyl)pyrimidine-2-carboxamide (105 mg, 0.33 mmol) was added methyl iodide (50 mg, 0.33 mmol) dropwise. The resulting mixture was stirred at r.t. for 18 h under an atmosphere of nitrogen. It was filtered to remove solids and purified directly with C18 column chromatography using water (0.05% NH₄HCO₃)/MeCN (2:1) as the mobile phase followed by reverse phase HPLC using the following conditions: (SHIMADZU HPLC) YMC-Actus Triart C18 ExRS column, 30*150 mm, 5 μm; mobile phase, water (10 mmol/L NH₄HCO_{3+0.1}% NH₃.H₂O) and ACN (25% ACN up to 55% in 8 min) to afford 4-(1H-imidazol-1-yl)-6-methoxy-N-((1r,4r)-4-methoxycyclohexyl)pyrimidine-2-carboxamide (27 mg, 0.082 mmol, 25%) as an off-white solid. LRMS (ES) m/z 332 (M+H). ¹H NMR (400 MHz, DMSO-d6) δ 8.96 (d, J=7.8 Hz, 1H), 8.52 (s, 1H), 7.93 (t, J=1.5 Hz, 1H), 7.14 (s, 1H), 6.91 (s, 1H), 3.79-3.67 (m, 1H), 3.43 (s, 3H), 3.24 (s, 3H), 3.19-3.09 (m, 1H), 2.19-1.79 (m, 4H), 1.44-1.19 (m, 4H).

Compounds 223, 272, 290, and 304 prepared using the methods provided in the table below.

| Compound | |
|---|---|
| Number | Method of Preparation |
| 223 | Beginning with (1r,3r)-3-aminocyclobutan-1-ol, prepared in the same fashion |
| | as Compound 351. |
| 272 | Beginning with 4-(1H-imidazol-1-yl)pyrimidine-2-carboxylic acid and 3-(6- |
| | (trifluoromethyl)pyridin-3-yl)cyclobutan-1-amine, prepared using same amide |
| | bond formation conditions as Compound 191 |
| 290 | Prepared from 4-(1H-imidazol-1-yl)pyrimidine-2-carboxylic acid using same |
| | amide bond conditions described for Compound 191. |
| 304 | Beginning with 4-chloropyrimidine-2-carboxylic acid, acyl chloride |
| | preparation and amide bond formation using same procedure as Compound |
| | 189, followed by nucleophilic aromatic substitution with imidazole using the |
| | same procedure as described for Compound 36. |

### Example AR

### Synthesis of Compound 262

### Preparation of 4-(1H-imidazol-1-yl)-N-(1-phenylazetidin-3-yl)pyrimidine-2-carboxamide

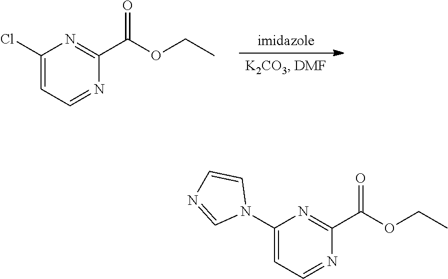

Step 1: Preparation of Ethyl 4-(1H-Imidazol-1-yl)pyrimidine-2-carboxylate. To a solution of ethyl 4-chloropyrimidine-2-carboxylate (2.0 g 10.8 mmol) in DMF (5 mL) was added potassium carbonate (3.0 g, 21.7 mmol), imidazole (811 mg, 11.9 mmol), stirred at 100° C. for 2 h, cooled to r.t., diluted with DCM (30 mL), and filtered through Celite to yield ethyl 4-(1H-imidazol-1-yl)pyrimidine-2-carboxylate (2.36 g, 10.8 mmol, quantitative yield). The material was used in the next reaction without further purification. LRMS (APCI) m/z 219.1 (M+H).

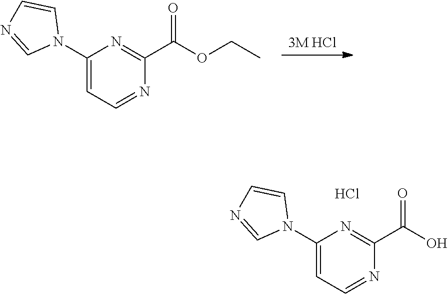 [SMILES: CCOC(=O)c1nccc(-n2ccnc2)n1 ; "3M HCl" ; O=C(O)c1nccc(-n2ccnc2)n1 ; Cl]

Step 2: Preparation of 4-(1H-Imidazol-1-yl)pyrimidine-2-carboxylic acid hydrochloride. A solution of ethyl 4-(1H-imidazol-1-yl)pyrimidine-2-carboxylate (2.3 g, 10.5 mmol) and 3 M aq. hydrochloric acid (5 mL) was stirred at 100° C. for 2 h, cooled, concentrated, sonicated in ether, and filtered to yield 4-(1H-imidazol-1-yl)pyrimidine-2-carboxylic acid hydrochloride (2.38 g, 10.5 mmol, quantitative yield). LRMS (ESI) m/z 191.0 (M+H).

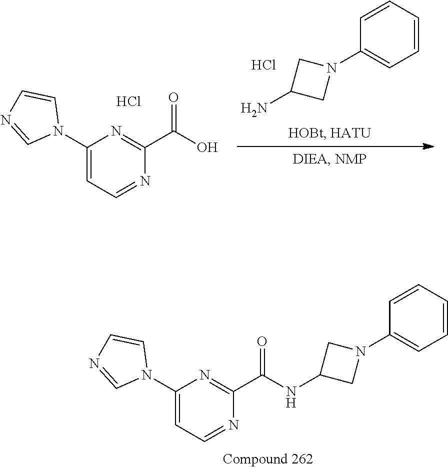 [SMILES: O=C(O)c1nccc(-n2ccnc2)n1 ; Cl ; Cl ; NC1CN(c2ccccc2)C1 ; O=C(NC1CN(c2ccccc2)C1)c1nccc(-n2ccnc2)n1]

Step 3: Preparation of 4-(1H-Imidazol-1-yl)-N-(1-phenylazetidin yl)pyrimidine-2-carboxamide. To a solution of 4-(1H-imidazol-1-yl)pyrimidine-2-carboxylic acid hydrochloride (120 mg, 0.53 mmol) and DIEA (0.3 mL, 1.72 mmol) in NMP (1 mL) were added HOBt (107 mg, 0.79 mmol), HBTU (301 mg, 0.79 mmol), ethyl 4-(1H-imidazol-1-yl)pyrimidine-2-carboxylate-1-phenylazetidin-3-amine hydrochloride (234 mg, 1.06 mmol). The resulting mixture was stirred overnight, filtered and directly purified by reverse phase prep HPLC (Phenomenex Gemini 5 micron C18 Axia pack 150×21.2 mm column) using a gradient of 3-40% water/acetonitrile with 0.1% formic acid to yield 4-(1H-imidazol-1-yl)-N-(1-phenylazetidin-3-yl)pyrimidine-2-carboxamide (6.6 mg, 0.02 mmol, 4%) as a white solid. LRMS (ESI) m/z 321.2 (M+H). ¹H NMR (400 MHz, DMSO-d₆) δ 9.50 (d, J=7.7 Hz, 1H), 9.06 (d, J=5.6 Hz, 1H), 8.96 (s, 1H), 8.27 (s, 1H), 8.07 (d, J=5.7 Hz, 1H), 7.23-7.16 (m, 3H), 6.70 (t, J=7.3 Hz, 1H), 6.49 (d, J=8.0 Hz, 2H), 4.93 (sext, J=6.8 Hz, 1H), 4.20 (t, J=7.4 Hz, 2H), 3.88 (t, J=6.7 Hz, 2H).

Compound s 188 and 271 were prepared using the methods provided in the table below.

| Compound | |
|---|---|
| Number | Method of Preparation |
| 188 | Prepared in same fashion as Compound 189, except last |
| | step nucleophilic aromatic substitution with imidazole |
| | using same procedure as described Compound 36. |
| 271 | Beginning with 4-(1H-imidazol-1-yl)pyrimidine-2- |
| | carboxylic acid and 3-(2-(trifluoromethyl)pyridin- |
| | 4-yl)cyclobutan-1-amine, prepared using same amide |
| | bond formation conditions as Compound 191. |

### Example AS

### Synthesis of Compound 307

### Preparation of N-(6-(difluoromethyl)pyridin-3-yl)-6-(2-methoxyethoxy)-2-(1-methyl-1H-imidazol-5-yl)pyrimidine-4-carboxamide

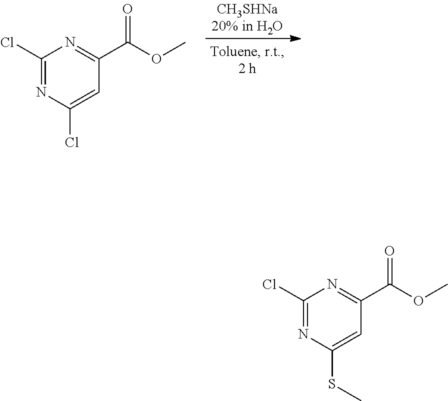

Step 1: Preparation of Methyl 2-chloro-6-(methylthio)pyrimidine-4-carboxylate. To a stirred solution of methyl 2,6-dichloropyrimidine-4-carboxylate (2.0 g, 9.66 mmol) in toluene (20 mL) was added CH₃SHNa (3.77 g, 10.63 mmol, 1.1 equiv, 20%). The resulting mixture was stirred at r.t. for 2 h and concentrated under reduced pressure to afford methyl 2-chloro-6-(methylthio)pyrimidine-4-carboxylate (2.0 g, 6.40 mmol, crude) as an off-white solid. LRMS (ES) m/z 219 (M+H).

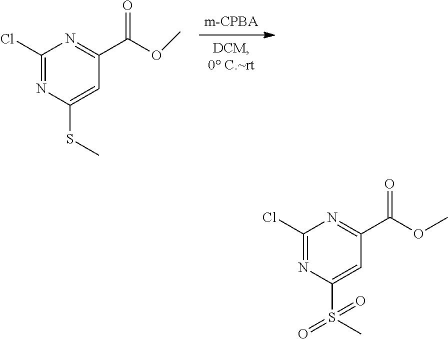

Step 2: Preparation of Methyl 2-chloro-6-(methylsulfonyl)pyrimidine-4-carboxylate: To a stirred solution of methyl 2-chloro-6-(methylthio)pyrimidine-4-carboxylate (2.0 g, 6.40 mmol, 1 equiv, 70%) in DCM (30 mL) at 0° C. was added m-CPBA (2.76 g, 16.01 mmol, 2.5 equiv). The resulting mixture was stirred at r.t. for 18 h, concentrated under reduced pressure, diluted with EtOAc (20 mL), combined with Na₂S₂O₃ (10 mL) and extracted twice with EtOAc (50 mL). The organic phases were combined, washed with brine, dried over sodium sulfate, concentrated under reduced pressure and purified with silica gel using 40% petroleum ether/EtOAc to afford methyl 2-chloro-6-(methylsulfonyl)pyrimidine-4-carboxylate (960 mg, 3.83 mmol, 60%) as a white solid. LRMS (ES) m/z 251 (M+H).

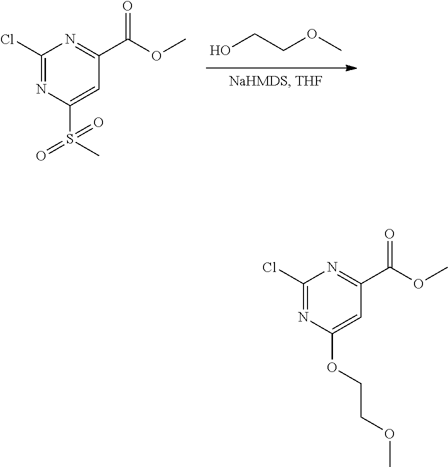

Step 3: Preparation of Methyl 2-chloro-6-(2-methoxyethoxy)pyrimidine-4-carboxylate: To a stirred solution of 2-methoxyethanol (273 mg, 3.59 mmol) in THF (10 mL) was added NaHMDS (1.80 mL, 3.59 mmol, 1 equiv). The resulting mixture was stirred at r.t. for 30 min, cooled to 0° C. and added to a solution of methyl 2-chloro-6-(methylsulfonyl)pyrimidine-4-carboxylate (900 mg, 3.59 mmol, 1 equiv) in THF (5 mL) at 0° C. dropwise over 5 min. The resulting mixture was stirred at 0° C. for 1 h, the pH was adjusted to 7 with AcOH, quenched with water (10 mL) at 0° C. and extracted twice with EtOAc (25 mL). The organic extracts were combined, washed with brine, dried over sodium sulfate and concentrated under reduced pressure to provide crude methyl 2-chloro-6-(2-methoxyethoxy)pyrimidine-4-carboxylate (900 mg, 3.65 mmol) as a yellow oil LRMS (ES) m/z 247 (M+H).

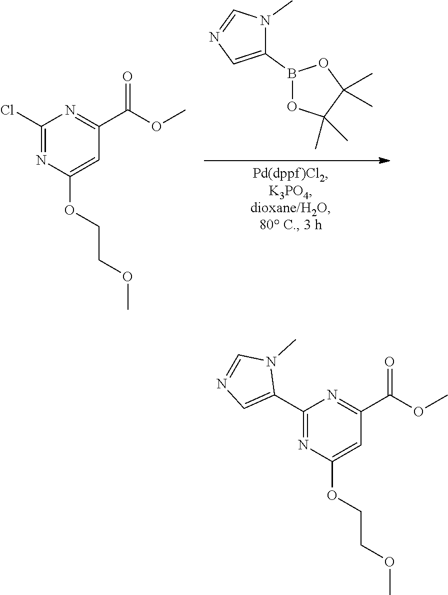 [SMILES: COCCOc1cc(C(=O)OC)nc(Cl)n1 ; Cn1cncc1B1OC(C)(C)C(C)(C)O1 ; COCCOc1cc(C(=O)OC)nc(-c2cncn2C)n1]

Step 4: Preparation of Methyl 6-(2-methoxyethoxy)-2-(1-methyl-1H-imidazol-5-yl)pyrimidine-4-carboxylate: Prepared using same Suzuki coupling conditions as described for Compound 347 and purified using C18 column chromatography, eluting with water (0.05% NH₄HCO₃)/MeCN (2:1) to provide methyl 6-(2-methoxyethoxy)-2-(1-methyl-1H-imidazol-5-yl)pyrimidine-4-carboxylate (500 mg, 1.71 mmol, 46%) as a yellow solid LRMS (ES) m/z 293 (M+H).

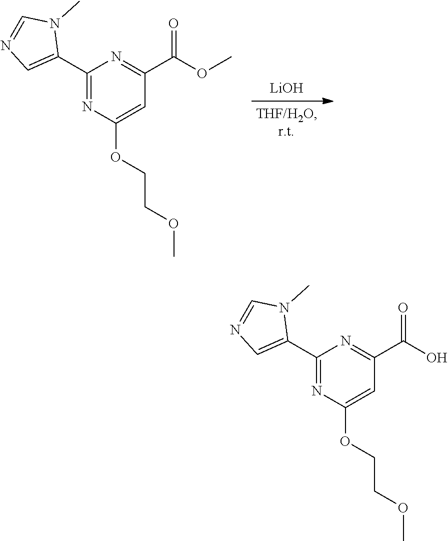 [SMILES: COCCOc1cc(C(=O)OC)nc(-c2cncn2C)n1 ; "LiOH" ; COCCOc1cc(C(=O)O)nc(-c2cncn2C)n1]

Step 5: Preparation of 6-(2-Methoxyethoxy)-2-(1-methyl-1H-imidazol-5-yl)pyrimidine-4-carboxylic acid: Prepared using same ester hydrolysis conditions as described for Compound 347 to afford crude 6-(2-methoxyethoxy)-2-(1-methyl-1H-imidazol-5-yl)pyrimidine-4-carboxylic acid (950 mg, 3.41 mmol) as a yellow solid. LRMS (ES) m/z 279 (M+H).

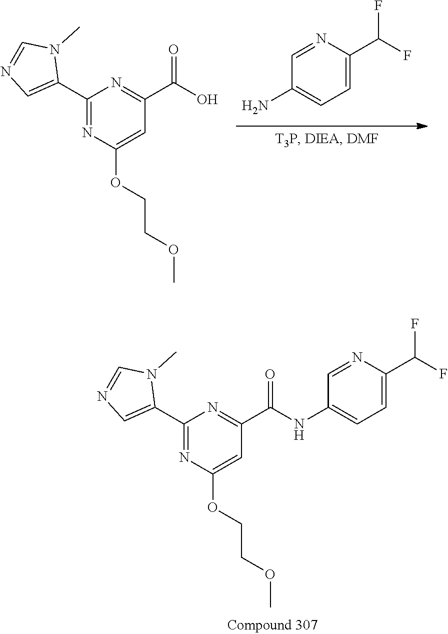 [SMILES: COCCOc1cc(C(=O)O)nc(-c2cncn2C)n1 ; Nc1ccc(C(F)F)nc1 ; COCCOc1cc(C(=O)Nc2ccc(C(F)F)nc2)nc(-c2cncn2C)n1]

Step 6: Preparation of N-(6-(difluoromethyl)pyridin-3-yl)-6-(2-methoxyethoxy)-2-(1-methyl-1H-imidazol-5-yl)pyrimidine-4-carboxamide Prepared using same amide bond formation conditions as described for Compound 191 and purified with preparative HPLC using the following conditions (SHIMADZU HPLC); Column, XBridge Prep OBD C18 Column, 30*150 mm, 5 μm; mobile phase, water (10 mmol/L NH₄HCO_{3+0.1}% NH₃.H₂O) and ACN (30% ACN up to 50% in 8 min) to afford N-(6-(difluoromethyl)pyridin-3-yl)-6-(2-methoxyethoxy)-2-(1-methyl-1H-imidazol-5-yl)pyrimidine-4-carboxamide (35 mg, 0.087 mmol 20%) as an off-white solid. LRMS (ES) m/z 405 [M+H]. ¹H NMR (400 MHz, DMSO-d6) δ 10.79 (s, 1H), 9.15 (d, J=2.5 Hz, 1H), 8.50 (dd, J=8.5, 2.5 Hz, 1H), 8.23 (d, J=1.2 Hz, 1H), 7.91 (s, 1H), 7.78 (d, J=8.6 Hz, 1H), 7.26 (s, 1H), 6.97 (t, J=55.1 Hz, 1H), 4.65-4.58 (m, 2H), 4.10 (s, 3H), 3.78-3.71 (m, 2H), 3.33 (s, 3H).

Compound 338 was prepared using the methods provided in the table below.

| Compound | |
|---|---|
| Number | Method of Preparation |
| 338 | Beginning with 2-chloro-5-fluoropyrimidine-4-carboxylic |
| | acid, amide bond formation as Compound 189 followed by |
| | Suzuki coupling in an oil bath at 120° C. for 30 |
| | min as described Compound 59. |

### Example AT

### Synthesis of Compound 282

### Preparation of N-(6-(difluoromethoxy)pyridin-3-yl)-2-(1-methyl-1H-imidazol-5-yl)-6-(tetrahydro-2H-pyran-4-yl)pyrimidine-4-carboxamide

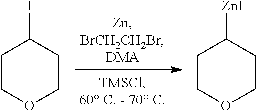

Step 1: Preparation of (Tetrahydro-2H-pyran-4-yl)zinc(II) iodide. To a stirred mixture of Zn (7.40 g, 113.1 mmol, 1.20 equiv) in DMA (200 mL) were added 1,2-dibromoethane (1.77 g, 9.43 mmol, 0.10 equiv) and TMSCl (1.23 g, 11.32 mmol, 0.12 equiv) dropwise at r.t. under nitrogen atmosphere. The resulting mixture was stirred for 20 min at 60° C. under a nitrogen atmosphere, cooled to r.t. and 4-iodooxane (20 g, 94.3 mmol, 1 equiv) in DMA (10 ml) was added dropwise over 2 min at r.t. The resulting mixture was stirred for additional 0.5 h at 70° C., cooled to r.t. and then used in the next step directly without further purification. LRMS (ES) m/z 277[M+H].

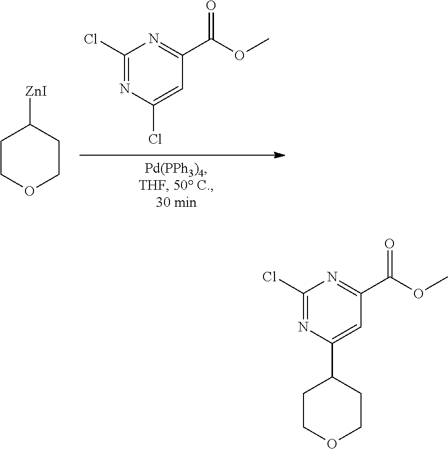

Step 2: Preparation of Methyl 2-chloro-6-(tetrahydro-2H-pyran-4-yl)pyrimidine-4-carboxylate: To a stirred solution of methyl 2,6-dichloropyrimidine-4-carboxylate (10.0 g, 48.31 mmol) and Pd(PPh₃)₄ (7.8 g, 6.76 mmol) in THF (200 mL) were added (tetrahydro-2H-pyran-4-yl)zinc(II) iodide (the solution obtained in the above step) dropwise at r.t. under a nitrogen atmosphere. The resulting mixture was stirred for 1 h at 50° C. under a nitrogen atmosphere. Water (200 mL) was added and the resulting mixture was extracted with EtOAc (3×150 mL). The organic layers were combined, washed with brine, dried over anhydrous sodium sulfate, concentrated in vacuo and purified with silica gel using 20% EtOAc/petroleum ether to afford methyl 2-chloro-6-(tetrahydro-2H-pyran-4-yl)pyrimidine-4-carboxylate (6.80 g, 26.49 mmol 55%) as a yellow solid. LRMS (ES) m/z 257 [M+H].

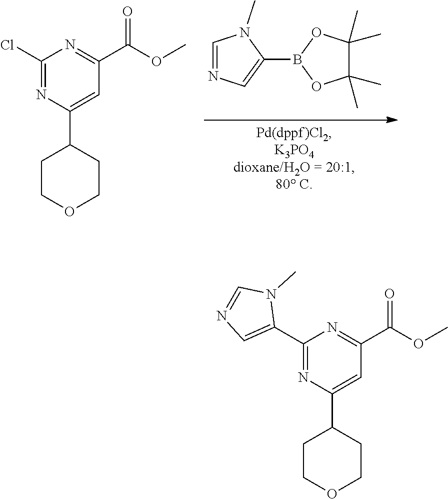

Step 3: Preparation of Methyl 2-(1-methyl-1H-imidazol-5-yl)-6-(tetrahydro-2H-pyran-4-yl)pyrimidine-4-carboxylate: Prepared using the same Suzuki coupling conditions described for Compound 347 and purified with silica gel using 10% MeOH/DCM to provide methyl 2-(1-methyl-1H-imidazol-5-yl)-6-(tetrahydro-2H-pyran-4-yl)pyrimidine-4-carboxylate (8.0 g, 26.5 mmol, 76%) as a faintly yellow solid. LRMS (ES) m/z 303[M+H].

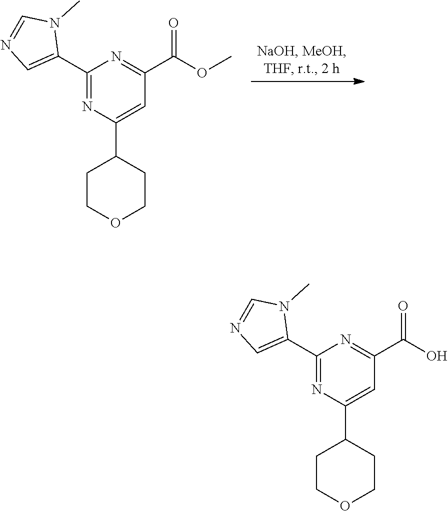 [SMILES: COC(=O)c1cc(C2CCOCC2)nc(-c2cncn2C)n1 ; Cn1cncc1-c1nc(C(=O)O)cc(C2CCOCC2)n1]

Step 4: Preparation of 2-(1-Methyl-1H-imidazol-5-yl)-6-(tetrahydro-2H-pyran-4-yl)pyrimidine-4-carboxylic acid. Methyl 2-(1-methyl-1H-imidazol-5-yl)-6-(tetrahydro-2H-pyran-4-yl)pyrimidine-4-carboxylate (6.0 g, 19.9 mmol) was dissolved in MeOH (30 mL) and THF (30 mL). Water (10 mL) was added followed by NaOH (1.5 g, 37.5 mmol) and the resulting mixture was stirred at r.t. for 2 h. The mixture was acidified to pH 3 using 1 M aq. HCl, concentrated and purified with C18 column chromatography, eluting with water/ACN (5-13% gradient in 10 min to provide 2-(1-methyl-1H-imidazol-5-yl)-6-(tetrahydro-2H-pyran-4-yl)pyrimidine-4-carboxylic acid (5.03 g, 17.4 mmol, 87%) as a white solid. LRMS (ES) m/z 289[M+H]. ¹H NMR (300 MHz, DMSO-d6) δ 7.89 (d, J=1.2 Hz, 1H), 7.82 (d, J=1.2 Hz, 1H), 7.69 (s, 1H), 4.07 (s, 3H), 3.98 (ddd, J=11.4, 4.3, 2.0 Hz, 2H), 3.47 (td, J=11.4, 2.8 Hz, 2H), 3.09 (tt, J=11.1, 4.3 Hz, 1H), 1.94-1.70 (m, 4H).

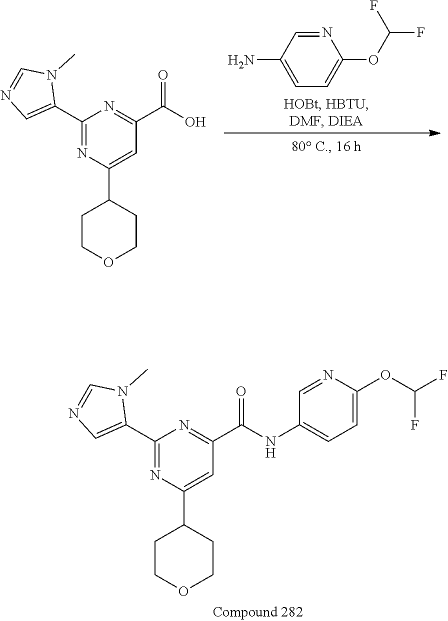 [SMILES: Cn1cncc1-c1nc(C(=O)O)cc(C2CCOCC2)n1 ; Nc1ccc(OC(F)F)nc1 ; Cn1cncc1-c1nc(C(=O)Nc2ccc(OC(F)F)nc2)cc(C2CCOCC2)n1]

Step 5: Preparation of N-(6-(difluoromethoxy)pyridin-3-yl)-2-(1-methyl-1H-imidazol-5-yl)-6-(tetrahydro-2H-pyran-4-yl)pyrimidine-4-carboxamide. Prepared in an oil bath at 80° C. for 16 h using same amide bond formation conditions as Compound 351 to provide N-(6-(difluoromethoxy)pyridin-3-yl)-2-(1-methyl-1H-imidazol-5-yl)-6-(tetrahydro-2H-pyran yl)pyrimidine-4-carboxamide (48 mg, 0.11 mmol, 32%) as a white solid. LRMS (APCI) m/z 431.1 (M+H). ¹H NMR (400 MHz, DMSO-d₆) δ 10.73 (s, 1H), 8.74 (d, J=2.5 Hz, 1H), 8.35 (dd, J=8.7, 2.5 Hz, 1H), 8.21 (s, 1H), 7.94-7.49 (m, 3H), 7.19 (d, J=8.9 Hz, 1H), 4.11 (s, 3H), 4.04-3.94 (m, 2H), 3.49 (t, J=11.5 Hz, 2H), 3.15 (t, J=9.5 Hz, 1H), 1.96-1.74 (m, 4H).

Compound 267 was prepared using the methods provided in the table below.

| Compound | |
|---|---|
| Number | Method of Preparation |
| 267 | Prepared in the same fashion as Compound 307 |

### Example AU

### Synthesis of Compound 348

### Preparation of N-(6-(difluoromethyl)pyridin-3-yl)-2-(1-methyl-1H-imidazol-5-yl)-6-(2-oxaspiro[3.3]heptan-6-yl)pyrimidine-4-carboxamide

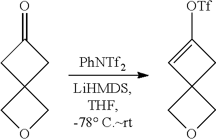 [SMILES: "PhNTf2" ; O=C1CC2(COC2)C1 ; O=S(=O)(OC1=CC2(COC2)C1)C(F)(F)F]

Step 1: Preparation of 2-Oxaspiro[3.3]hept-5-en-6-yl trifluoromethanesulfonate. To a stirred solution of 2-oxaspiro[3.3]heptan-6-one (4.0 g, 35.67 mmol) in THF (40 mL) at −78° C. was added LiHMDS (1M in THF, 42.8 mL, 42.81 mmol) dropwise over 20 min under a nitrogen atmosphere. After stirring at −78° C. for 30 min, to the above mixture at −78° C. was added a solution of 1,1,1-trifluoro-N-phenyl-N-trifluoromethanesulfonylmethanesulfonamide (15.3 g, 42.81 mmol) in THF (20 mL) dropwise over 10 min. The resulting mixture was stirred at r.t. for 18 h, water (50 mL) was added and extracted twice with pentane (50 mL). The combined organic layers were washed with brine, dried over sodium sulfate and concentrated under reduced pressure to give crude 2-oxaspiro[3.3]hept-5-en-6-yl trifluoromethanesulfonate (9.5 g, 38.9 mmol) as a red oil. LC/MS signal was not observed.

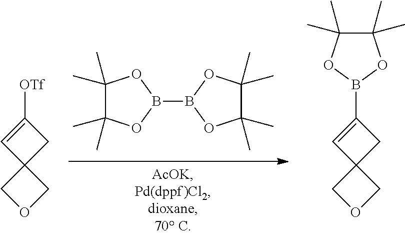

Step 2: Preparation of 4,4,5,5-Tetramethyl-2-(2-oxaspiro[3.3]hept-5-en-6-yl)-1,3,2-dioxaborolane. To a stirred solution of 2-oxaspiro[3.3]hept-5-en-6-yl trifluoromethanesulfonate (9.5 g, 38.91 mmol) and bis(pinacolato)diboron (9.88 g, 38.91 mmol) in dioxane (100 mL) were added KOAc (7.64 g, 77.81 mmol) and Pd(dppf)Cl₂.CH₂Cl₂ (3.17 g, 3.89 mmol). The resulting mixture was stirred at 70° C. for 2 h, cooled to r.t., concentrated under reduced pressure and purified with silica gel using 10% EtOAc/petroleum ether to provide 4,4,5,5-tetramethyl-2-(2-oxaspiro[3.3]hept-5-en-6-yl)-1,3,2-dioxaborolane (8.0 g, 36.0 mmol, 93%) as a yellow oil. LC/MS signal was not observed.

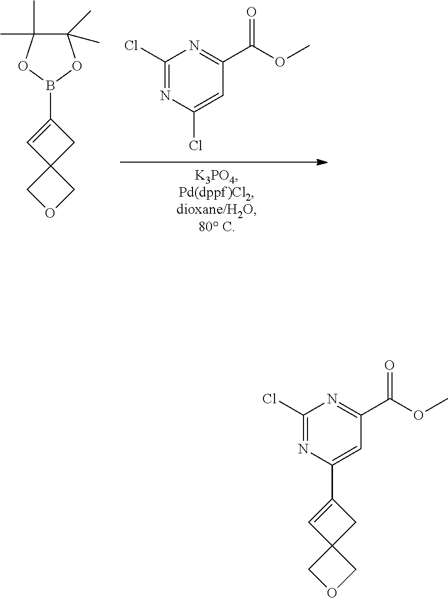

Step 3: Preparation of Methyl 2-chloro-6-(2-oxaspiro[3.3]hept-5-en-6-yl)pyrimidine-4-carboxylate: Prepared by heating at 80° C. for 4 h using the same Suzuki coupling conditions described for Compound 347 and purified with silica gel using 30% EtOAc/petroleum ether to provide methyl 2-chloro-6-(2-oxaspiro[3.3]hept-5-en-6-yl)pyrimidine-4-carboxylate (3.8 g, 14.2 mmol, 48%) as a yellow solid. LRMS (ES) m/z 267 [M+H].

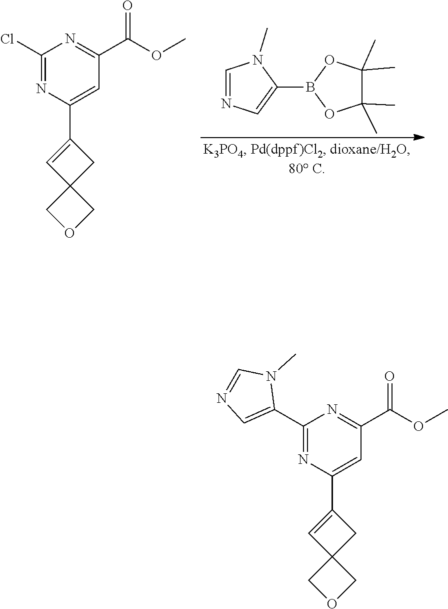 [SMILES: COC(=O)c1cc(C2=CC3(COC3)C2)nc(Cl)n1 ; Cn1cncc1B1OC(C)(C)C(C)(C)O1 ; COC(=O)c1cc(C2=CC3(COC3)C2)nc(-c2cncn2C)n1]

Step 4: Preparation of Methyl 2-(1-methyl-1H-imidazol-5-yl)-6-(2-oxaspiro[3.3]hept-5-en-6-yl)pyrimidine-4-carboxylate. Prepared by heating at 80° C. for 4 h using the same Suzuki coupling conditions described for Compound 347 and purified with silica gel using 10% MeOH/DCM to provide methyl 2-(1-methyl-1H-imidazol-5-yl)-6-(2-oxaspiro[3.3]hept-5-en-6-yl)pyrimidine-4-carboxylate (680 mg, 2.18 mmol, 39%) as a faintly green solid. LRMS (ES) m/z 313 [M+H].

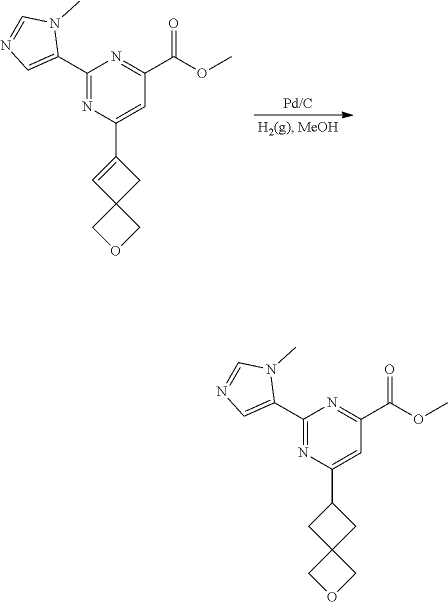 [SMILES: COC(=O)c1cc(C2=CC3(COC3)C2)nc(-c2cncn2C)n1 ; COC(=O)c1cc(C2CC3(COC3)C2)nc(-c2cncn2C)n1]

Step 5: Preparation of Methyl 2-(1-methyl-1H-imidazol-5-yl)-6-(2-oxaspiro[3.3]heptan-6-yl)pyrimidine-4-carboxylate: To a stirred solution of methyl 2-(1-methyl-1H-imidazol-5-yl)-6-(2-oxaspiro[3.3]hept-5-en-6-yl)pyrimidine-4-carboxylate (680 mg, 2.18 mmol) in MeOH (10 mL) was added Pd/C (10% Pd, 50% wet with water, 680 mg). The resulting mixture was stirred at r.t. for 1 h under an atmosphere of hydrogen. It was filtered and concentrated under reduced pressure to give methyl 2-(1-methyl-1H-imidazol-5-yl)-6-(2-oxaspiro[3.3]heptan-6-yl)pyrimidine-4-carboxylate (630 mg, 2.00 mmol, 92%) as a brown oil. LRMS (ES) m/z 315 [M+H].

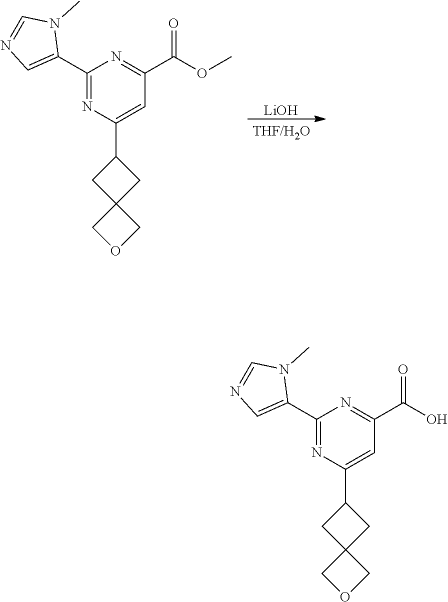 [SMILES: COC(=O)c1cc(C2CC3(COC3)C2)nc(-c2cncn2C)n1 ; "LiOH" ; Cn1cncc1-c1nc(C(=O)O)cc(C2CC3(COC3)C2)n1]

Step 6: Preparation of 2-(1-Methyl-1H-imidazol-5-yl)-6-(2-oxaspiro[3.3]heptan-6-yl)pyrimidine-4-carboxylic acid. Prepared using same ester hydrolysis conditions as described for Compound 347 to provide crude 2-(1-methyl-1H-imidazol-5-yl)-6-(2-oxaspiro[3.3]heptan-6-yl)pyrimidine-4-carboxylic acid (600 mg, 2.0 mmol) as a brown oil. LRMS (ES) m/z 301 [M+H].

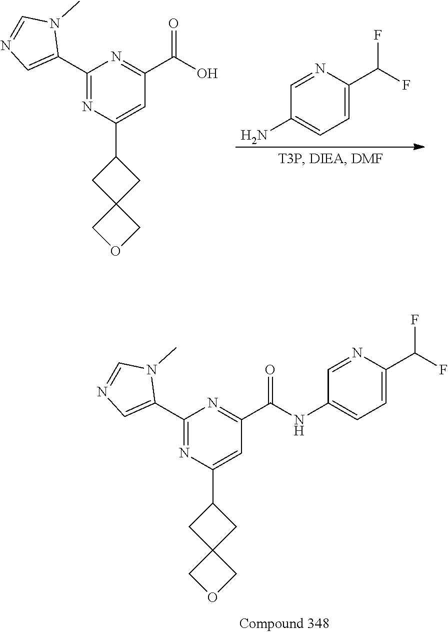 [SMILES: Cn1cncc1-c1nc(C(=O)O)cc(C2CC3(COC3)C2)n1 ; Nc1ccc(C(F)F)nc1 ; Cn1cncc1-c1nc(C(=O)Nc2ccc(C(F)F)nc2)cc(C2CC3(COC3)C2)n1]

Step 7: Preparation of N-(6-(difluoromethyl)pyridin-3-yl)-2-(1-methyl-1H-imidazol-5-yl)-6-(2-oxaspiro[3.3]heptan-6-yl)pyrimidine-4-carboxamide Prepared using same amide bond formation conditions as Compound 191 and purified using reverse phase HPLC with the following conditions: (SHIMADZU HPLC) Column, XBridge Prep OBD C18 Column, 30*150 mm, 5 μm; mobile phase, water (10 mmol/LNH₄HCO₃+0.1% NH₃.H₂O) and ACN (25% ACN up to 55% in 8 min) to provide N-(6-(difluoromethyl)pyridin-3-yl)-2-(1-methyl-1H-imidazol-5-yl)-6-(2-oxaspiro[3.3]heptan-6-yl)pyrimidine-4-carboxamide (44 mg, 0.10 mmol, 31%) as a faintly yellow solid. LRMS (ES) m/z 427 [M+H]. ¹H NMR (300 MHz, DMSO-d6) δ 10.86 (s, 1H), 9.14 (d, J=2.4 Hz, 1H), 8.50 (dd, J=8.5, 2.5 Hz, 1H), 8.19 (d, J=1.2 Hz, 1H), 7.91 (s, 1H), 7.83-7.70 (m, 2H), 6.97 (t, J=55.1 Hz, 1H), 4.71 (s, 2H), 4.55 (s, 2H), 4.11 (s, 3H), 3.65 (p, J=8.4 Hz, 1H), 2.75-2.62 (m, 2H), 2.61-2.53 (m, 2H).

### Example AV

### Synthesis of Compound 287

### Preparation of 6-(4,4-difluorocyclohexyl)-N-(6-(difluoromethyl)pyridin-3-yl)-2-(1-methyl-1H-imidazol-5-yl)pyrimidine-4-carboxamide

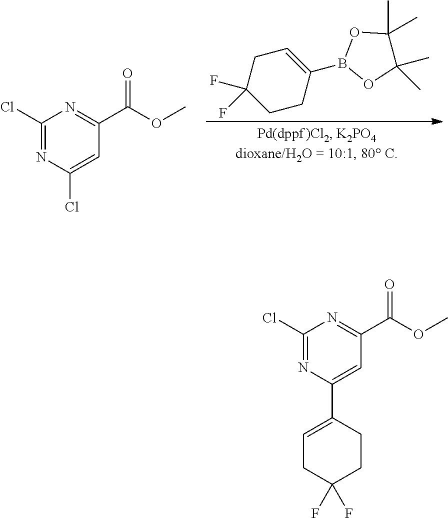

Step 1: Preparation of Methyl 2-chloro-6-(4,4-difluorocyclohex-1-en-1-yl)pyrimidine-4-carboxylate: Prepared by heating at 80° C. for 3 h using the same Suzuki coupling conditions described for Compound 347 and purified with silica gel using 10% EtOAc/petroleum ether to provide methyl 2-chloro-6-(4,4-difluorocyclohex-1-en-1-yl)pyrimidine-4-carboxylate (2.5 g, 8.66 mmol, 90%) as a yellow oil. LRMS (ES) m/z 289 (M+H).

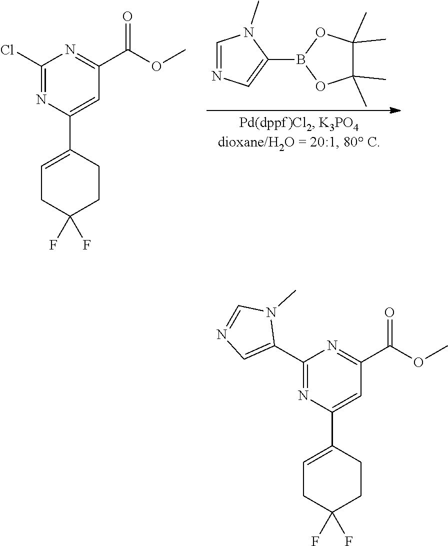

Step 2: Preparation of Methyl 6-(4,4-difluorocyclohex-1-en-1-yl)-2-(1-methyl-1H-imidazol-5-yl)pyrimidine-4-carboxylate. Prepared by heating at 80° C. for 2 h using the same Suzuki coupling conditions described for Compound 347 and purified with silica gel using 100% EtOAc to provide methyl 6-(4,4-difluorocyclohex-1-en-1-yl)-2-(1-methyl-1H-imidazol-5-yl)pyrimidine-4-carboxylate (1.9 g, 5.68 mmol, 66%) as a brown oil. LRMS (ES) m/z 335 (M+H).

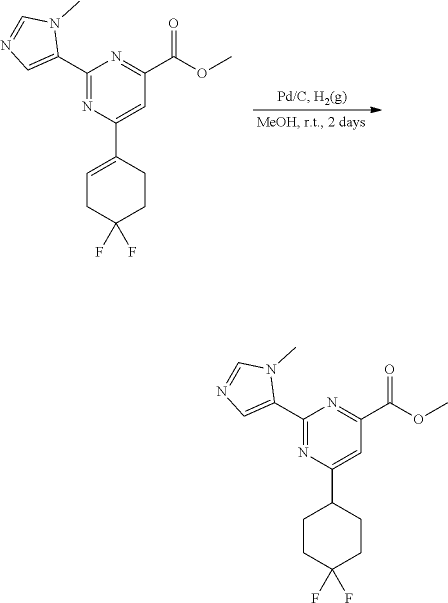 [SMILES: COC(=O)c1cc(C2=CCC(F)(F)CC2)nc(-c2cncn2C)n1 ; COC(=O)c1cc(C2CCC(F)(F)CC2)nc(-c2cncn2C)n1]

Step 3: Preparation of Methyl 6-(4,4-difluorocyclohexyl)-2-(1-methyl-1H-imidazol-5-yl)pyrimidine-4-carboxylate. To a solution of methyl 6-(4,4-difluorocyclohex-1-en-1-yl)-2-(1-methyl-1H-imidazol-5-yl)pyrimidine-4-carboxylate (1.8 g, 5.38 mmol) in MeOH (30 mL) was added Pd/C (10% Pd, 50% wet with water, 1.8 g). The resulting mixture was stirred under balloon pressure hydrogen at r.t. for 2 days, filtered through celite and concentrated under reduced pressure to give crude methyl 6-(4,4-difluorocyclohexyl)-2-(1-methyl-1H-imidazol-5-yl)pyrimidine-4-carboxylate (1.4 g, 4.16 mmol). LRMS (ES) m/z 337 (M+H).

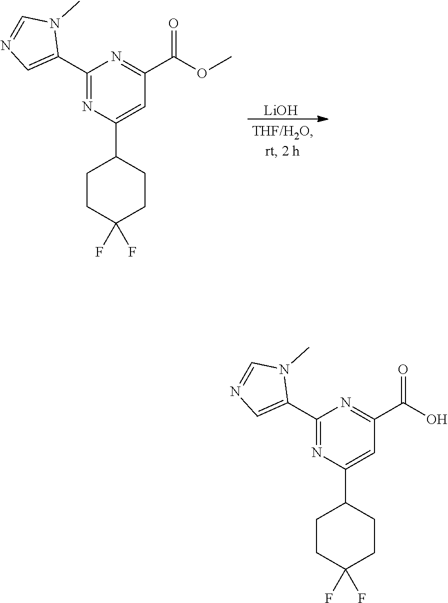 [SMILES: COC(=O)c1cc(C2CCC(F)(F)CC2)nc(-c2cncn2C)n1 ; "LiOH" ; Cn1cncc1-c1nc(C(=O)O)cc(C2CCC(F)(F)CC2)n1]

Step 4: Preparation of 6-(4,4-Difluorocyclohexyl)-2-(1-methyl-1H-imidazol-5-yl)pyrimidine-4-carboxylic acid. Prepared using same ester hydrolysis conditions as described for Compound 347 to provide 6-(4,4-difluorocyclohexyl)-2-(1-methyl-1H-imidazol-5-yl)pyrimidine-4-carboxylic acid (1.3 g, 4.0 mmol, 85% purity) as a yellow solid LRMS (ES) m/z 323 (M+H).

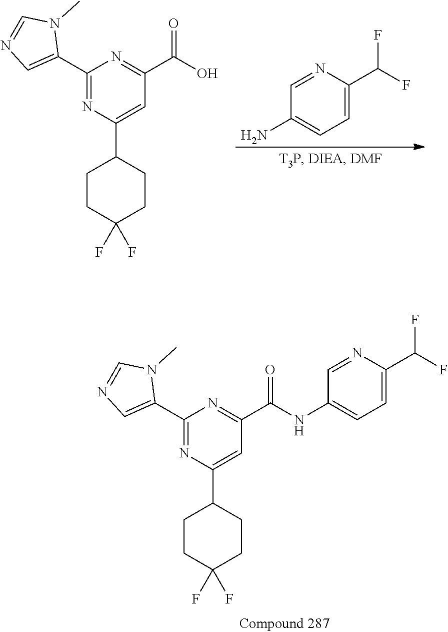 [SMILES: Cn1cncc1-c1nc(C(=O)O)cc(C2CCC(F)(F)CC2)n1 ; Nc1ccc(C(F)F)nc1 ; Cn1cncc1-c1nc(C(=O)Nc2ccc(C(F)F)nc2)cc(C2CCC(F)(F)CC2)n1]

Step 5: Preparation of 6-(4,4-Difluorocyclohexyl)-N-(6-(difluoromethyl)pyridin-3-yl)-2-(1-methyl-1H-imidazol-5-yl)pyrimidine-4-carboxamide. Prepared using same amide bond formation conditions as Compound 191 and purified using reverse phase HPLC with the following conditions: (SHIMADZU HPLC) Column, XBridge Prep OBD C18 Column, 30*150 mm, 5 μm; mobile phase, water (10 mmol/L NH₄HCO₃+0.1% NH₃.H₂O) and ACN (30% ACN up to 60% in 8 min) to provide 6-(4,4-difluorocyclohexyl)-N-(6-(difluoromethyl)pyridin-3-yl)-2-(1-methyl-1H-imidazol-5-yl)pyrimidine-4-carboxamide (62 mg, 0.094 mmol, 43%) as an off-white solid. LRMS (ES) m/z 449 [M+H]. ¹H NMR (300 MHz, DMSO-d6) δ 10.87 (s, 1H), 9.15 (d, J=2.4 Hz, 1H), 8.50 (dd, J=8.5, 2.4 Hz, 1H), 8.25-8.18 (m, 1H), 7.90 (s, 1H), 7.86-7.74 (m, 2H), 6.97 (t, J=55.1 Hz, 1H), 4.10 (s, 3H), 3.10 (t, J=11.5 Hz, 1H), 2.25-1.99 (m, 6H), 1.99-1.75 (m, 2H).

Compounds 198, 326, 345, and 355 were prepared using the methods provided in the table below.

| Compound | |
|---|---|
| Number | Method of Preparation |
| 198 | Prepared in same fashion as Compound 282 |
| 326 | Beginning with 2-chloro-5-methylpyrimidine-4-carboxylic |
| | acid and 6-(trifluoromethyl)pyridin-3-amine, amide bond |
| | formation performed in same fashion as Compound 189 and |
| | Suzuki coupling performed in the same fashion as |
| | Compound 321. |
| 345 | Prepared in same fashion as Compound 348 |
| 355 | Prepared in same fashion as Compound 282 |

### Example AW

### Synthesis of Compound 281

### Preparation of N-((1r,4r)-4-(difluoromethoxy)cyclohexyl)-2-(1-methyl-1H-imidazol-5-yl) (tetrahydro-2H-pyran-4-yl)pyrimidine-4-carboxamide

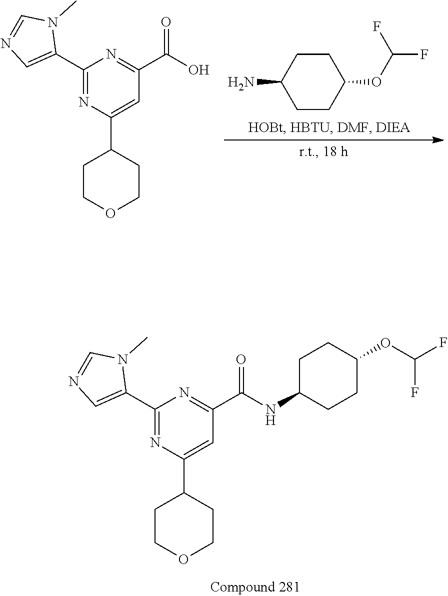 [SMILES: Cn1cncc1-c1nc(C(=O)O)cc(C2CCOCC2)n1 ; N[C@H]1CC[C@H](OC(F)F)CC1 ; Cn1cncc1-c1nc(C(=O)N[C@H]2CC[C@H](OC(F)F)CC2)cc(C2CCOCC2)n1]

Preparation of N-((1r,4r)-4-(difluoromethoxy)cyclohexyl)-2-(1-methyl-1H-imidazol-5-yl)-6-(tetrahydro-2H-pyran-4-yl)pyrimidine-4-carboxamide Prepared at r.t. for 18 h using the same procedure as described for Compound 282 and purified by reverse phase prep HPLC (Phenomenex Gemini 5 micron C18 Axia pack 150×21.2 mm column) with a gradient of 3-40% water/acetonitrile with 0.1% formic acid to yield to provide N-((1r,4r)-4-(difluoromethoxy)cyclohexyl)-2-(1-methyl-1H-imidazol-5-yl)-6-(tetrahydro-2H-pyran-4-yl)pyrimidine-4-carboxamide (92 mg, 0.35 mmol, 61%) as a white solid. LRMS (APCI) m/z 436.2 (M+H). ¹H NMR (400 MHz, DMSO-d₆) δ 8.52 (d, J=8.4 Hz, 1H), 8.07 (s, 1H), 7.86 (s, 1H), 7.67 (s, 1H), 6.74 (t, J=76.6 Hz, 1H), 4.11-4.01 (m, 4H), 4.01-3.89 (m, 2H), 3.90-3.76 (m, 1H), 3.47 (t, J=11.4 Hz, 2H), 3.16-3.03 (m, 1H), 2.06-1.94 (m, 2H), 1.94-1.72 (m, 6H), 1.69-1.41 (m, 4H).

Compounds 197, 230, 291, and 309 were prepared using the methods provided in the table below.

| Compound | |
|---|---|
| Number | Method of Preparation |
| 197 | Prepared in same fashion as Compound 282 |
| 230 | Prepared in same fashion as Compound 282 |
| 291 | Prepared in same fashion as Compound 282 |
| 309 | Prepared in same fashion as Compound 295 |

### Example AX

### Synthesis of Compound 289

### Preparation of 6-(4,4-difluorocyclohexyl)-N-((1r,4r)-4-methoxycyclohexyl)-2-(1-methyl-1H-imidazol-5-yl)pyrimidine-4-carboxamide

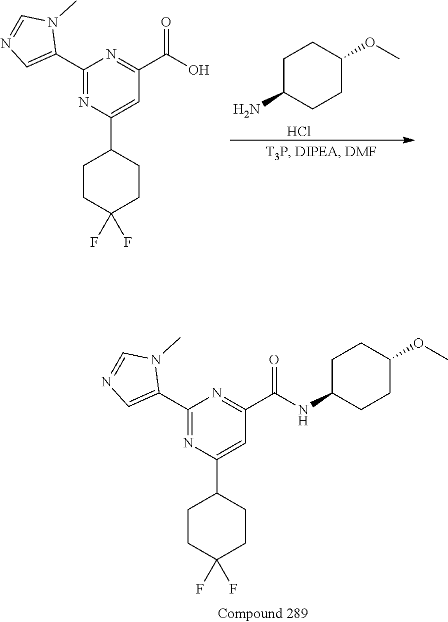

Preparation of 6-(4,4-Difluorocyclohexyl)-N-((1r,4r)-4-methoxycyclohexyl)-2-(1-methyl-1H-imidazol-5-yl)pyrimidine-4-carboxamide: Prepared using same amide bond formation conditions as Compound 191 and purified using reverse phase HPLC with the following conditions: (SHIMADZU HPLC) Column, XBridge Prep OBD C18 Column, 30*150 mm, 5 μm; mobile phase, water (10 mmol/L NH₄HCO₃+0.1% NH₃.H₂O) and ACN (30% ACN up to 60% in 8 min) to provide 6-(4,4-difluorocyclohexyl)-N-((1r,4r)-4-methoxycyclohexyl)-2-(1-methyl-1H-imidazol-5-yl)pyrimidine-4-carboxamide (61 mg, 0.14 mmol, 44%) as an off-white solid. LRMS (ES) m/z 434 [M+H]. ¹H NMR (300 MHz, DMSO-d₆) δ 8.51 (d, J=8.5 Hz, 1H), 8.07 (d, J=1.2 Hz, 1H), 7.86 (s, 1H), 7.69 (s, 1H), 4.05 (s, 3H), 3.80 (d, J=12.4 Hz, 1H), 3.25 (s, 3H), 3.19-2.97 (m, 2H), 2.03 (d, J=9.7 Hz, 7H), 1.85 (d, J=12.6 Hz, 5H), 1.54 (q, J=13.1, 12.2 Hz, 2H), 1.32-1.14 (m, 2H).

Compounds 227, 277, 285, 286, 288, 337, and 341 were prepared using the methods provided in the table below.

| Compound | |
|---|---|
| Number | Method of Preparation |
| 227 | Prepared in same fashion as Compound 282 |
| 277 | Prepared in same fashion as Compound 282 |
| 285 | Prepared in same fashion as Compound 287 |
| 286 | Prepared in same fashion as Compound 287 |
| 288 | Prepared in same fashion as Compound 287 |
| 337 | Prepared in same fashion as Compound 282 |
| 341 | Beginning with 2-chloro-5-fluoropyrimidine-4-carboxylic |
| | acid, amide bond formation as Compound 189 followed by |
| | Suzuki coupling in an oil bath at 120° C. for 30 min |
| | as described Compound 59. |

### Example AY

### Synthesis of Compound 248

### Preparation of N-(6-(2-hydroxypropan-2-yl)pyridin-3-yl)-2-(1H-imidazol-1-yl)pyrimidine-4-carboxamide

Step 1: Preparation of 2-bromo-N-(6-(2-hydroxypropan-2-yl)pyridin-3-yl)pyrimidine-4-carboxamide: Prepared with 2-bromopyrimidine-4-carboxylic acid and 2-(5-aminopyridin-2-yl)propan-2-ol, amide bond formation was performed in same fashion as Compound 189.

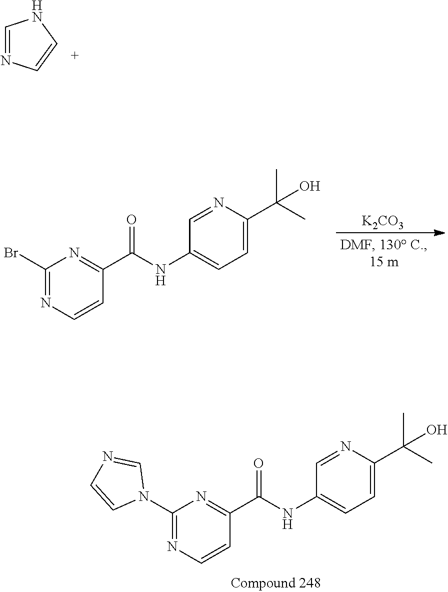

Step 2: Preparation of N-(6-(2-hydroxypropan-2-yl)pyridin-3-yl)-2-(1H-imidazol-1-yl)pyrimidine-4-carboxamide. 2-bromo-N-(6-(2-hydroxypropan-2-yl)pyridin-3-yl)pyrimidine-4-carboxamide (72 mg, 0.21 mmol) was combined with imidazole (44 mg, 0.64 mmol) and potassium carbonate (89 mg, 0.64 mmol) and dissolved in DMF (2 mL). The reaction was heated at 130° C. in the microwave for 15 min. It was filtered through a syringe filter and purified using reverse phase HPLC with a 40 minute gradient from 0-100% ACN/water (Phenomenex Gemini 5 micron C18 column), yielding N-(6-(2-hydroxypropan-2-yl)pyridin-3-yl)-2-(1H-imidazol-1-yl)pyrimidine-4-carboxamide (10 mg, 0.031 mmol, 14%) as a white solid. LRMS (APCI) m/z 325.1 (M+H). ¹H NMR (400 MHz, DMSO-d₆) δ 10.85 (s, 1H), 9.16 (d, J=5.0 Hz, 1H), 9.04 (s, 1H), 8.91 (d, J=2.5 Hz, 1H), 8.31 (s, 1H), 8.19 (dd, J=8.6, 2.5 Hz, 1H), 8.05 (d, J=5.0 Hz, 1H), 7.72 (d, J=8.6 Hz, 1H), 7.22 (s, 1H), 5.25 (s, 1H), 1.47 (s, 6H).

Compound 280 was prepared using the methods provided in the table below.

| Compound | |
|---|---|
| Number | Method of Preparation |
| 280 | Prepared in same fashion as Compound 282 |

### Example AZ

### Synthesis of Compound 332

### Preparation of N-(6-(2-hydroxypropan-2-yl)pyridin-3-yl)-2-(1-methyl-1H-imidazol-5-yl) (tetrahydro-2H-pyran-4-yl)pyrimidine-4-carboxamide

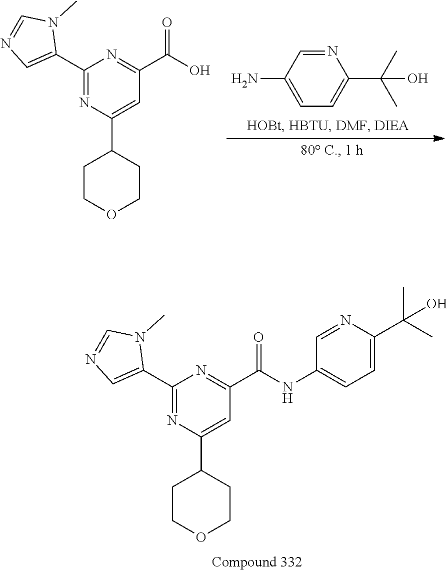 [SMILES: Cn1cncc1-c1nc(C(=O)O)cc(C2CCOCC2)n1 ; CC(C)(O)c1ccc(N)cn1 ; Cn1cncc1-c1nc(C(=O)Nc2ccc(C(C)(C)O)nc2)cc(C2CCOCC2)n1]

Preparation of N-(6-(2-hydroxypropan-2-yl)pyridin-3-yl)-2-(1-methyl-1H-imidazol-5-yl)-6-(tetrahydro-2H-pyran-4-yl)pyrimidine-4-carboxamide Prepared in same fashion as Compound 282 with amide bond formation at 80° C. for 1 h and purified with silica gel using 10% MeOH/DCM followed by reverse phase prep HPLC (Phenomenex Gemini 5 micron C18 Axia pack 150×21.2 mm column) with a gradient of 3-40% water/acetonitrile with 0.1% formic acid to yield to provide N-(6-(2-hydroxypropan-2-yl)pyridin-3-yl)-2-(1-methyl-1H-imidazol-5-yl)-6-(tetrahydro-2H-pyran-4-yl)pyrimidine-4-carboxamide (39 mg, 0.092 mmol, 18%) as a white solid. LRMS (APCI) m/z 423.1 (M+H). ¹H NMR (400 MHz, DMSO-d₆) δ 10.63 (s, 1H), 8.91 (d, J=2.4 Hz, 1H), 8.26-8.16 (m, 2H), 7.91 (s, 1H), 7.81 (d, J=1.5 Hz, 1H), 7.69 (d, J=8.6 Hz, 1H), 5.22 (s, 1H), 4.11 (s, 3H), 4.03-3.91 (m, 2H), 3.55-3.42 (m, 2H), 3.22-3.07 (m, 1H), 1.96-1.73 (m, 4H), 1.45 (d, J=1.4 Hz, 6H).

Compounds 200, 229, and 340 were prepared using the methods provided in the table below.

| Compound | |
|---|---|
| Number | Method of Preparation |
| 200 | Prepared in same fashion as Compound 282 |
| 229 | Prepared in same fashion as Compound 282 |
| 340 | Beginning with 2-chloro-5-fluoropyrimidine-4-carboxylic |
| | acid, amide bond formation as Compound 189 followed by |
| | Suzuki coupling in an oil bath at 120° C. for 30 min as |
| | described Compound 59. |

### Example BA

### Synthesis of Compound 336

### Preparation of 6-(4,4-difluorocyclohexyl)-N-(6-(2-hydroxypropan-2-yl)pyridin-3-yl)-2-(1-methyl-1H-imidazol-5-yl)pyrimidine-4-carboxamide

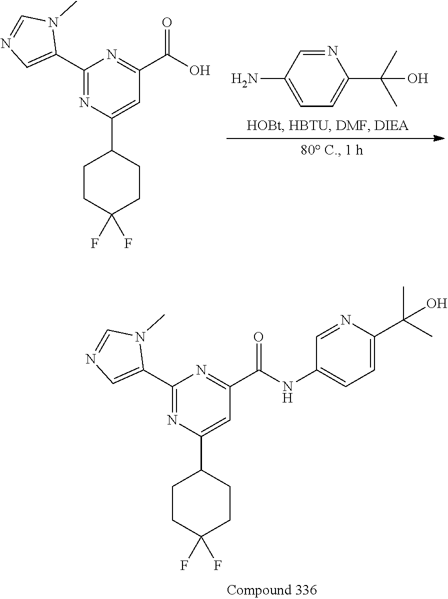 [SMILES: Cn1cncc1-c1nc(C(=O)O)cc(C2CCC(F)(F)CC2)n1 ; CC(C)(O)c1ccc(N)cn1 ; Cn1cncc1-c1nc(C(=O)Nc2ccc(C(C)(C)O)nc2)cc(C2CCC(F)(F)CC2)n1]

Preparation of 6-(4,4-Difluorocyclohexyl)-N-(6-(2-hydroxypropan-2-yl)pyridin-3-yl)-2-(1-methyl-1H-imidazol-5-yl)pyrimidine-4-carboxamide: Synthesized in the same fashion as Compound 287 with amide bond formation as described for Compound 282 at 80° C. for 1 h and purified using reverse phase prep HPLC (Phenomenex Gemini 5 micron C18 Axia pack 150×21.2 mm column) with a gradient of 3-40% water/acetonitrile with 0.1% formic acid to yield 6-(4,4-difluorocyclohexyl)-N-(6-(2-hydroxypropan-2-yl)pyridin-3-yl)-2-(1-methyl-1H-imidazol-5-yl)pyrimidine-4-carboxamide (7 mg, 0.015 mmol, 7% over 2 steps). LRMS (ESI) m/z 457.0 (M+H). ¹H NMR (400 MHz, DMSO-d₆) δ 10.63 (s, 1H), 8.90 (s, 1H), 8.19 (s, 1H), 8.15 (s, 1H), 7.89 (s, 1H), 7.82 (s, 1H), 7.69 (d, J=8.6 Hz, 1H), 5.21 (s, 1H), 4.09 (s, 3H), 3.09 (t, J=11.7 Hz, 1H), 2.19-1.80 (m, 8H), 1.45 (s, 6H).

Compounds 311, 333, and 334 were prepared using the methods provided in the table below.

| Compound | |
|---|---|
| Number | Method of Preparation |
| 311 | Synthesized in same fashion as Compound 295. |
| 333 | Beginning with 2-chloro-5-fluoropyrimidine-4-carboxylic |
| | acid, amide bond formation as Compound 189 followed by |
| | Suzuki coupling in an oil bath at 120 C. for 30 min as |
| | described Compound 59. |
| 334 | Synthesized in same fashion as Compound 282 with amide |
| | bond formation at r.t. for 15 min. |

### Example BB

### Synthesis of Compound 315 and Compound 316

### Preparation of N-((1r,4R)-4-methoxycyclohexyl)-6-((1s,4S)-4-methoxycyclohexyl)-2-(1-methyl-1H-imidazol-5-yl)pyrimidine-4-carboxamide and N,6-bis((1r,4R)-4-methoxycyclohexyl)-2-(1-methyl-1H-imidazol-5-yl)pyrimidine-4-carboxamide

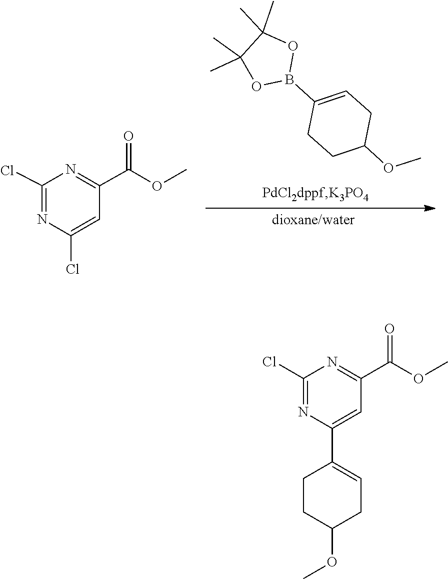

Step 1: Preparation of Methyl 2-chloro-6-(4-methoxycyclohex-1-en-1-yl)pyrimidine-4-carboxylate. To a solution of methyl 2,6-dichloropyrimidine-4-carboxylate (600 mg, 2.90 mmol) in 1,4-dioxane (7.5 mL) was added PdCl₂dppf (106 mg, 0.15 mmol) and 2-(4-methoxycyclohex-1-en-1-yl)-4,4,5,5-tetramethyl-1,3,2-dioxaborolane (690 mg, 2.90 mmol), followed by potassium phosphate tribasic (1.23 g, 5.80 mmol) in water (2.5 mL). The reaction was stirred in oil bath at 80° C. for 2.5 h, cooled, filtered through celite and directly purified by silica gel using 10% MeOH/DCM to yield methyl 2-chloro-6-(4-methoxycyclohex-1-en-1-yl)pyrimidine-4-carboxylate (485 mg, 1.72 mmol, 59%) as an off white solid. LRMS (ESI) m/z 283.0 (M+H).

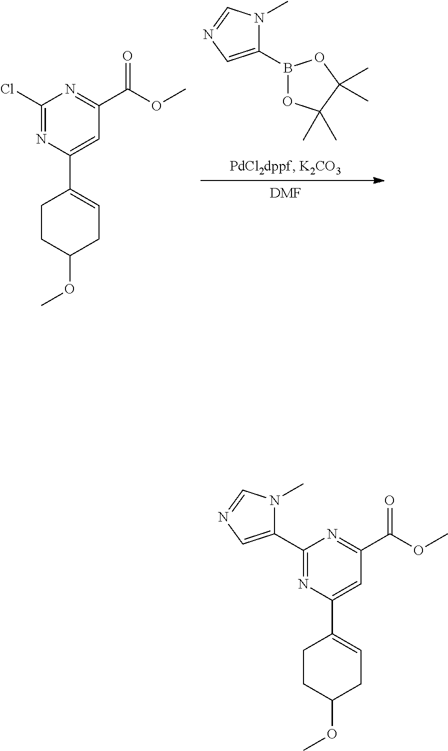

Step 2: Preparation of Methyl 6-(4-methoxycyclohex-1-en-1-yl)-2-(1-methyl-1H-imidazol-5-yl)pyrimidine-4-carboxylate. To a solution of methyl 2-chloro-6-(4-methoxycyclohex-1-en-1-yl)pyrimidine-4-carboxylate (485 mg, 1.72 mmol) in dimethylformamide (5 mL) was added 1-methyl-5-(4,4,5,5-tetramethyl-1,3,2-dioxaborolan-2-yl)imidazole (375 mg, 1.80 mmol), potassium carbonate (474 mg, 3.43 mmol), and PdCl₂dppf (63 mg, 0.09 mmol). The reaction vial was capped and stirred at 120° C. on heating block for 40 min, cooled, diluted with DCM, filtered through celite, concentrated and directly purified by silica gel chromatography using 10% MeOH/DCM to give methyl 6-(4-methoxycyclohex-1-en-1-yl)-2-(1-methyl-1H-imidazol-5-yl)pyrimidine-4-carboxylate (248 mg, 0.78 mmol, 44%) as an off-white solid. LRMS (ESI) m/z 329.0 (M+H).

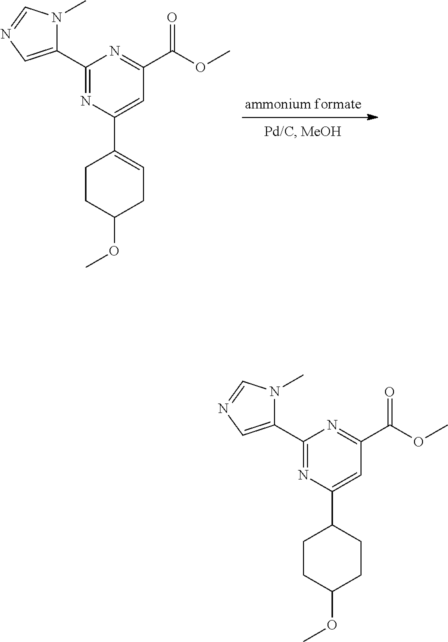 [SMILES: COC(=O)c1cc(C2=CCC(OC)CC2)nc(-c2cncn2C)n1 ; COC(=O)c1cc(C2CCC(OC)CC2)nc(-c2cncn2C)n1]

Step 3: Preparation of Methyl 6-(4-methoxycyclohexyl)-2-(1-methyl-1H-imidazol-5-yl)pyrimidine-4-carboxylate. A solution of methyl 6-(4-methoxycyclohex-1-en-1-yl)-2-(1-methyl-1H-imidazol-5-yl)pyrimidine-4-carboxylate (248 mg, 0.76 mmol) in methanol (5 mL) was purged with nitrogen for 5 min, added 5% palladium on activated charcoal (248 mg, 0.27 mmol), purged with nitrogen for 5 min, added ammonium formate (238 mg, 3.78 mmol), capped, stirred at 70° C. in an oil bath for 1 h, cooled to r.t., filtered through celite, concentrated under reduced pressure and directly purified by silica gel chromatography using 10% MeOH/DCM to yield methyl 6-(4-methoxycyclohexyl)-2-(1-methyl-1H-imidazol-5-yl)pyrimidine-4-carboxylate (134 mg, 0.41 mmol, 54%) as an off-white solid and mixture of diastereomers (˜4:1 by ¹H NMR) with the cis isomer being the major product. LRMS (ESI) m/z 331.0 (M+H).

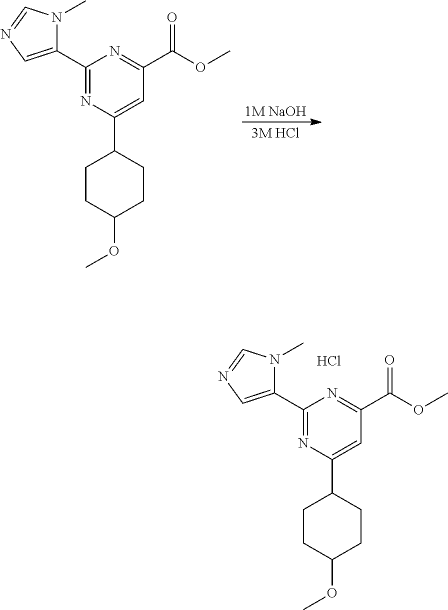

Step 4: Preparation of 6-(4-Methoxycyclohexyl)-2-(1-methyl-1H-imidazol-5-yl)pyrimidine-4-carboxylic acid hydrochloride. A solution of methyl 6-(4-methoxycyclohexyl)-2-(1-methyl-1H-imidazol-5-yl)pyrimidine-4-carboxylate (134 mg, 0.41 mmol) in 1 M aqueous sodium hydroxide (1.62 mL, 1.62 mmol) and MeOH (1 mL) was stirred at r.t. for 10 min, acidified with 3M HCl and concentrated to yield 6-(4-methoxycyclohexyl)-2-(1-methyl-1H-imidazol-5-yl)pyrimidine-4-carboxylic acid hydrochloride (127 mg, 0.40 mmol, 99%) as a white solid in quantitative yield as a mixture of diastereomers. LRMS (ESI) m/z 317.0 (M+H).

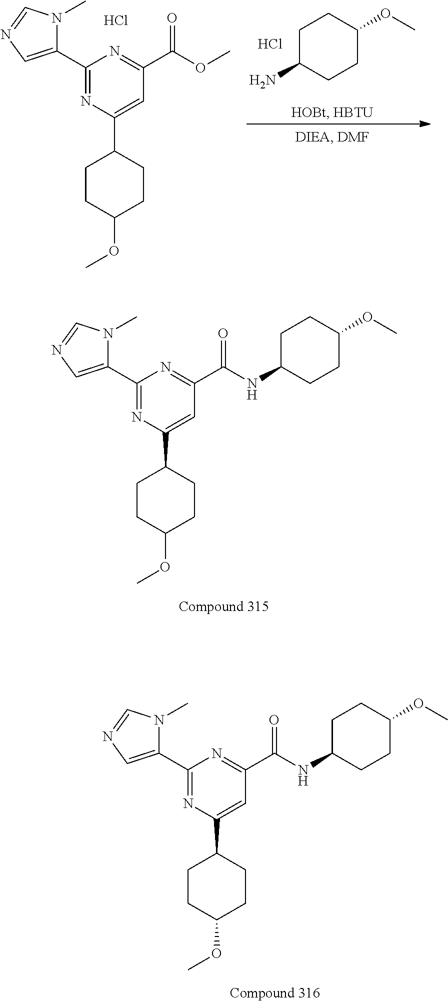

Step 5: Preparation of N-((1r,4R)-4-methoxycyclohexyl)-6-((1s,4S)-4-methoxycyclohexyl)-2-(1-methyl-1H-imidazol-5-yl)pyrimidine-4-carboxamide and N,6-bis((1r,4R)-4-methoxycyclohexyl)-2-(1-methyl-1H-imidazol-5-yl)pyrimidine-4-carboxamide. To a solution of 6-(4-methoxycyclohexyl)-2-(1-methyl-1H-imidazol-5-yl)pyrimidine-4-carboxylic acid hydrochloride (127 mg, 0.40 mmol) and DIEA (0.28 mL, 1.61 mmol) in DMF (1 mL) was added HOBt (81.4 mg, 0.60 mmol), HBTU (228 mg, 0.60 mmol), and (1r,4r)-4-methoxycyclohexan-1-amine (67 mg, 0.52 mmol). The resulting mixture was stirred in a sealed tube at 80° C. for 1 h followed by r.t. overnight. The reaction was diluted with water (20 mL), DCM (20 mL), and extracted with DCM (2×20 mL). The combined organic layers were dried over sodium sulfate, concentrated and purified by silica gel chromatography using a 0-10% MeOH/DCM gradient, followed by reverse phase prep HPLC (Phenomenex Gemini 5 micron C18 Axia pack 150×21.2 mm column) using a gradient of 3-40% water/acetonitrile with 0.1% formic acid to yield both N-((1r,4R)-4-methoxycyclohexyl)-6-((1s,4S)-4-methoxycyclohexyl)-2-(1-methyl-1H-imidazol-5-yl)pyrimidine-4-carboxamide (54 mg, 0.13 mmol, 31%) and N,6-bis((1r,4R)-4-methoxycyclohexyl)-2-(1-methyl-1H-imidazol-5-yl)pyrimidine-4-carboxamide (12 mg, 0.03 mmol, 7%) as white solids. N-((1r,4R)-4-methoxycyclohexyl)-6-((1s,4S)-4-methoxycyclohexyl)-2-(1-methyl-1H-imidazol-5-yl)pyrimidine-4-carboxamide. LRMS (APCI) m/z 428.4 (M+H). ¹H NMR (400 MHz, DMSO-d₆) δ 8.47 (d, J=8.5 Hz, 1H), 8.04 (s, 1H), 7.86 (s, 1H), 7.62 (s, 1H), 4.05 (s, 3H), 3.86-3.75 (m, 1H), 3.47 (p, J=2.9 Hz, 1H), 3.25 (s, 3H), 3.24 (s, 3H), 3.13 (ddd, J=14.5, 10.1, 3.8 Hz, 1H), 2.88 (qd, J=7.4, 3.7 Hz, 1H), 2.07-1.98 (m, 2H), 1.97-1.90 (m, 2H), 1.89-1.77 (m, 4H)), 1.74-1.66 (m, 2H), 1.63-1.47 (m, 4H), 1.29-1.15 (m, 2H). N,6-bis((1r,4R)-4-methoxycyclohexyl)-2-(1-methyl-1H-imidazol-5-yl)pyrimidine-4-carboxamide. LRMS (APCI) m/z 428.4 (M+H). ¹H NMR (400 MHz, DMSO-d₆) δ 8.47 (d, J=8.5 Hz, 1H), 8.05 (s, 1H), 7.85 (s, 1H), 7.64 (s, 1H), 4.04 (s, 3H), 3.87-3.74 (m, 1H), 3.27 (s, 3H), 3.25 (s, 3H), 3.22-3.07 (m, 2H), 2.80 (tt, J=11.8, 3.4 Hz, 1H), 2.15-2.09 (m, 2H), 2.08-1.94 (m, 4H), 1.88-1.79 (m, 2H), 1.68-1.46 (m, 4H), 1.25 (pd, J=13.2, 3.4 Hz, 4H).

Compounds 195 and 300 were prepared using the methods provided in the table below.

| Compound | |
|---|---|
| Number | Method of Preparation |
| 195 | Beginning with methyl 2,6-dichloropyrimidine-4- |
| | carboxylate, Negishi coupling using the same conditions as |
| | described for Compound 282, followed by ester hydrolysis |
| | and amide bond formation using the same conditions as |
| | Compound 189 and nucleophilic aromatic substitution with |
| | imidazole using the same conditions as described for |
| | Compound 248. |
| 300 | Synthesized in same fashion as Compound 282 with amide |
| | bond formation at r.t. for 15 min. |

### Example BC

### Synthesis of Compound 199

### Preparation of N-((1r,4r)-4-methoxycyclohexyl)-2-(1-methyl-1H-imidazol-5-yl)-6-(tetrahydro-2H-pyran-4-yl)pyrimidine-4-carboxamide

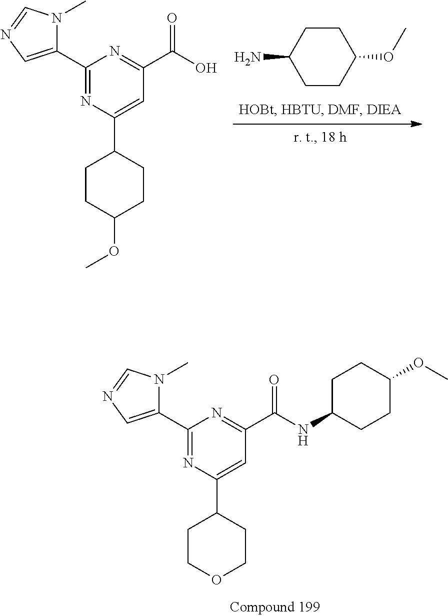

Preparation of N-((1r,4r)-4-methoxycyclohexyl)-2-(1-methyl-1H-imidazol-5-yl)-6-(tetrahydro-2H-pyran-4-yl)pyrimidine-4-carboxamide: Prepared using the same procedure as Compound 282 and purified by reverse phase prep HPLC (Phenomenex Gemini 5 micron C18 Axia pack 150×21.2 mm column) using a gradient of 0-40% water/acetonitrile with 0.1% formic acid to provide N-((1r,4r)-4-methoxycyclohexyl)-2-(1-methyl-1H-imidazol-5-yl)-6-(tetrahydro-2H-pyran-4-yl)pyrimidine-4-carboxamide (277 mg, 0.27 mmol, 38%) as a white solid. LRMS (APCI) m/z 400.0 (M+H). ¹H NMR (400 MHz, DMSO-d₆) δ 8.50 (d, J=8.5 Hz, 1H), 8.08 (s, 1H), 7.86 (s, 1H), 7.67 (s, 1H), 4.07 (s, 3H), 3.98 (dd, J=11.3, 4.2 Hz, 2H), 3.87-3.72 (m, 1H), 3.53-3.40 (m, 2H), 3.26 (s, 3H), 3.19-3.04 (m, 2H), 2.04 (d, J=12.4 Hz, 2H), 1.92-1.71 (m, 6H), 1.62-1.44 (m, 2H), 1.31-1.17 (m, 2H).

Compounds 228, 283, 324, and 339 were prepared using the methods provided in the table below.

| Compound | |
|---|---|
| Number | Method of Preparation |
| 228 | Synthesized in same fashion as Compound 282 with amide |
| | bond formation at r.t. for 15 min. |
| 283 | Synthesized in same fashion as Compound 282 with amide |
| | bond formation at r.t. for 18 h. |
| 324 | Synthesized in the same fashion as Compound 287 with |
| | amide bond formation at r.t. for 18 h as described for |
| | Compound 282. |
| 339 | Synthesized in the same fashion as Compound 287 with |
| | amide bond formation at r.t. for 18 h as described for |
| | Compound 282. |

### Example BD

### Synthesis of Compound 295

### Preparation of N-((1r,4r)-4-methoxycyclohexyl)-2-(1-methyl-1H-imidazol-5-yl)-6-(3-methyloxetan-3-yl)pyrimidine-4-carboxamide

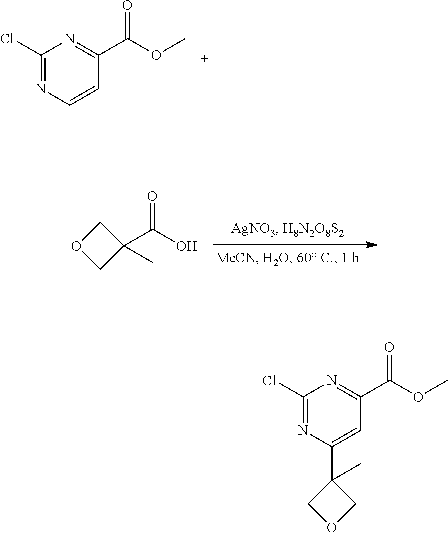

Step 1: Preparation of Methyl 2-chloro-6-(3-methyloxetan-3-yl)pyrimidine-4-carboxylate. Tetrahedron Letters 56 (2015) 4063-4066). To methyl 2-chloropyrimidine-4-carboxylate (500 mg, 2.90 mmol, 1 equiv), 3-methyloxetane-3-carboxylic acid (1.01 g, 8.69 mmol, 3 equiv), silver nitrate (1.97 g, 11.59 mmol, 4 equiv), and ammonium persulfate (3.31 g, 14.49 mmol, 5 equiv) was added a 1:1 mixture of acetonitrile and water (50 mL). The resulting mixture was heated at 60° C. for 1 h, cooled to r.t., quenched by addition of concentrated NH₄OH (10 mL), diluted with a saturated brine solution (10 mL), filtered through silica and extracted with ethyl acetate (3×50 mL). The organic layers were combined, washed with sodium bicarbonate, dried over sodium sulfate and concentrated in vacuo. The crude product was purified with silica gel using a 30% ethyl acetate/hexanes to give methyl 2-chloro-6-(3-methyloxetan-3-yl)pyrimidine-4-carboxylate as an off-white crystalline solid (0.573 g, 2.36 mmol, 82%). LRMS (APCI) m/z 243.4 (M+H). ¹H NMR (400 MHz, DMSO-d₆) δ 8.08 (s, 1H), 4.88 (d, J=6.0 Hz, 2H), 4.54 (d, J=6.0 Hz, 2H), 3.94 (s, 3H), 1.69 (s, 3H).

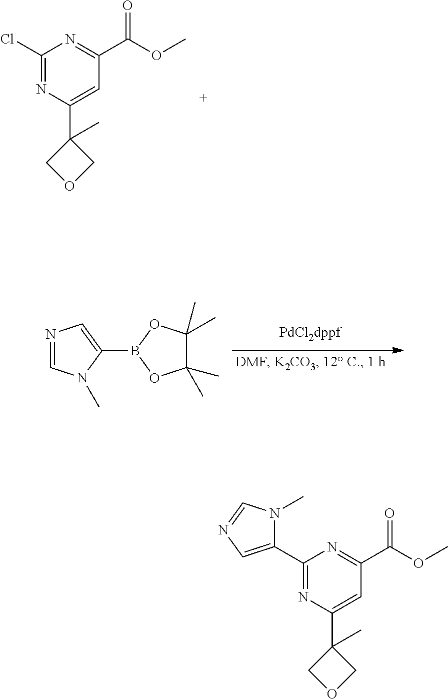 [SMILES: COC(=O)c1cc(C2(C)COC2)nc(Cl)n1 ; Cn1cncc1B1OC(C)(C)C(C)(C)O1 ; COC(=O)c1cc(C2(C)COC2)nc(-c2cncn2C)n1]

Step 2: Preparation of Methyl 2-(1-methyl-1H-imidazol-5-yl)-6-(3-methyloxetan-3-yl)pyrimidine-4-carboxylate: To methyl 2-chloro-6-(3-methyloxetan-3-yl)pyrimidine carboxylate (0.573 g, 2.36 mmol, 1 equiv.), 1-methyl-5-(4,4,5,5-tetramethyl-1,3,2-dioxaborolan-2-yl)imidazole (0.54 g, 2.597 mmol, 1.1 equiv.), potassium carbonate (0.653 g, 4.72 mmol, 2 equiv.) and PdCl₂(dppf) (0.173 g, 0.24 mmol, 0.1 equiv.) was added DMF (2 mL). The resulting mixture was heated at 120° C. for 1 h, concentrated under reduced pressure and purified with silica gel using 10% MeOH/DCM to give methyl 2-(1-methyl-1H-imidazol-5-yl)-6-(3-methyloxetan-3-yl)pyrimidine-4-carboxylate as an off-white solid (0.366 g, 1.27 mmol, 54%). LRMS (APCI) m/z 289.1 (M+H).

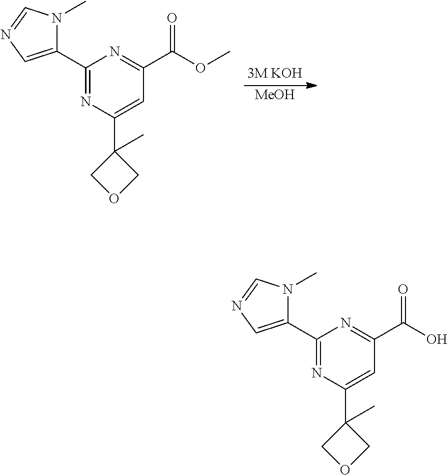 [SMILES: "MeOH" ; COC(=O)c1cc(C2(C)COC2)nc(-c2cncn2C)n1 ; Cn1cncc1-c1nc(C(=O)O)cc(C2(C)COC2)n1]

Step 3: Preparation of 2-(1-Methyl-1H-imidazol-5-yl)-6-(3-methyloxetan-3-yl)pyrimidine-4-carboxylic acid. To methyl 2-(3-methylimidazol-4-yl)-6-(3-methyloxetan-3-yl)pyrimidine-4-carboxylate (0.366 g, 1.269 mmol, 1 equiv.) was added MeOH (15 mL) followed by 3 M aq. KOH (0.84 mL, 2.52 mmol). The resulting mixture was stirred at r.t for 30 min, concentrated in vacuo, suspended in MeOH and filtered to give 2-(1-methyl-1H-imidazol-5-yl)-6-(3-methyloxetan-3-yl)pyrimidine-4-carboxylic acid as a yellow solid (0.115 g, 0.42 mmol, 33%). LRMS (APCI) m/z 275.1 (M+H).

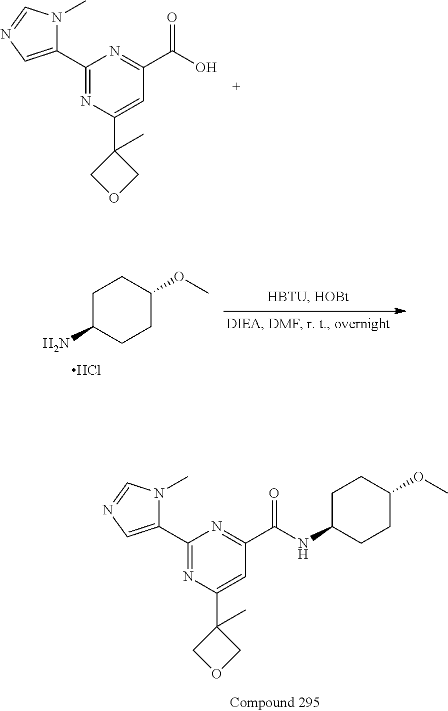

Step 4: Preparation of N-((1r,4r)-4-methoxycyclohexyl)-2-(1-methyl-1H-imidazol-5-yl)-6-(3-methyloxetan-3-yl)pyrimidine-4-carboxamide. To 2-(3-methylimidazol-4-yl)-6-(3-methyloxetan-3-yl)pyrimidine-4-carboxylic acid (100 mg, 0.37 mmol, 1 equiv), (1r,4r)-4-methoxycyclohexan-1-amine hydrochloride (0.06 g, 0.37 mmol, 1 equiv), O-(benzotriazol-1-yl)-N,N,N′,N′-tetramethyluronium hexafluorophosphate (0.207 g, 0.55 mmol, 1.5 equiv) and 1-hydroxybenzotriazole (0.074 g, 0.55 mmol, 1.5 equiv) was added DMF (4 mL). DIEA (0.637 mL, 3.65 mmol, 10 equiv) was added and the mixture was stirred at ambient temperature for 18 h. The product was purified using reverse phase HPLC with a 40 minute gradient from 5-100% ACN/water (Phenomenex Gemini 5-micron C18 Axia pack 150×21.2 mm column) to give N-((1r,4r)-4-methoxycyclohexyl)-2-(1-methyl-1H-imidazol-5-yl)-6-(3-methyloxetan-3-yl)pyrimidine-4-carboxamide (36 mg, 0.093 mmol, 26%) as a white solid. LRMS (APCI) m/z 386.2 (M+H). ¹H NMR (400 MHz, DMSO-d₆) δ 12.75 (s, 1H), 8.54 (d, J=8.5 Hz, 1H), 7.90 (s, 1H), 7.76 (s, 1H), 4.93 (d, 2H), 4.58 (d, 2H), 4.06 (s, 3H), 3.88-3.75 (m, 1H), 3.25 (s, 3H), 3.18-3.08 (m, 1H), 2.04 (d, J=14.7 Hz, 2H), 1.86 (d, J=11.0 Hz, 2H), 1.71 (s, 3H), 1.55 (q, J=13.0 Hz, 2H), 1.24 (q, J=12.9 Hz, 2H).

Compounds 196, 327, 342, 352, and 353 were prepared using the methods provided in the table below.

| Compound | |
|---|---|
| Number | Method of Preparation |
| 196 | Prepared in same fashion as Compound 195 |
| 327 | Prepared in same fashion as Compound 348 |
| 342 | Prepared in same fashion as Compound 287 with amide |
| | bond formation at Compound 282 |
| 352 | Prepared in same fashion as Compound 282 with amide |
| | bond formation at r.t. for 15 min. |
| 353 | Prepared in same fashion as Compound 282 with amide |
| | bond formation at r.t. for 15 min. |

### Example BE

### Synthesis of Compound 331

### Preparation of 6-(4,4-difluoro-1-hydroxycyclohexyl)-N-((1r,4r)-4-methoxycyclohexyl)-2-(1-methyl-1H-imidazol-5-yl)pyrimidine-4-carboxamide

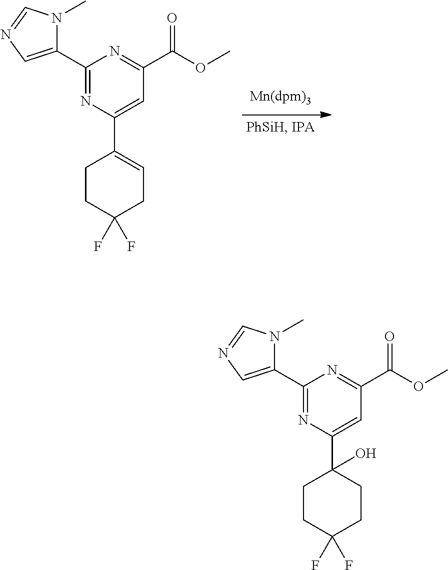 [SMILES: COC(=O)c1cc(C2=CCC(F)(F)CC2)nc(-c2cncn2C)n1 ; COC(=O)c1cc(C2(O)CCC(F)(F)CC2)nc(-c2cncn2C)n1]

Step 1: Preparation of Methyl 6-(4,4-difluoro-1-hydroxycyclohexyl)-2-(1-methyl-1H-imidazol-5-yl)pyrimidine-4-carboxylate. To a solution of methyl 6-(4,4-difluorocyclohex-1-en-1-yl)-2-(1-methyl-1H-imidazol-5-yl)pyrimidine-4-carboxylate (142 mg, 0.43 mmol) in isopropyl alcohol (1.4 mL) and DCM (0.1 mL) was added was added phenyl silane (0.11 mL, 0.85 mmol) and Mn(dpm)₃ (25.7 mg, 0.04 mmol). The reaction was stirred at r.t. open to air for 1 h, diluted with water (10 mL), saturated sodium bicarbonate (5 mL), DCM (10 mL), and extracted with DCM (2×20 mL). The combined organic layers were dried over sodium sulfate, concentrated, and purified by silica gel chromatography using 10% MeOH/DCM to yield methyl 6-(4,4-difluoro-1-hydroxycyclohexyl)-2-(1-methyl-1H-imidazol-5-yl)pyrimidine-4-carboxylate (67 mg, 0.19 mmol, 45%) as an off-white solid. LRMS (ESI) m/z 353.0 (M+H).

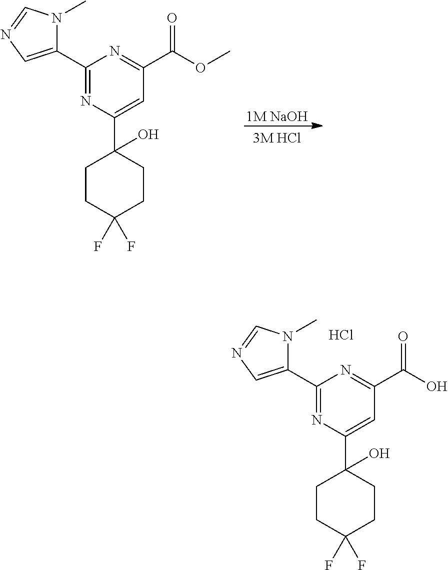

Step 2: Preparation of 6-(4,4-Difluoro-1-hydroxycyclohexyl)-2-(1-methyl-1H-imidazol-5-yl)pyrimidine-4-carboxylic acid hydrochloride. A solution of methyl 6-(4,4-difluoro-1-hydroxycyclohexyl)-2-(1-methyl-1H-imidazol-5-yl)pyrimidine-4-carboxylate (67 mg, 0.19 mmol) and 1 M aqueous sodium hydroxide (0.57 mL, 0.57 mmol) was stirred at r.t. for 1 h, acidified with 3 M aqueous hydrochloric acid, (0.32 mL, 0.95 mmol) and concentrated to afford 6-(4,4-difluoro-1-hydroxycyclohexyl)-2-(1-methyl-1H-imidazol-5-yl)pyrimidine-4-carboxylic acid hydrochloride (71 mg, 0.19 mmol, 99.6%) in quantitative yield. LRMS (ESI) m/z 339.0 (M+H).

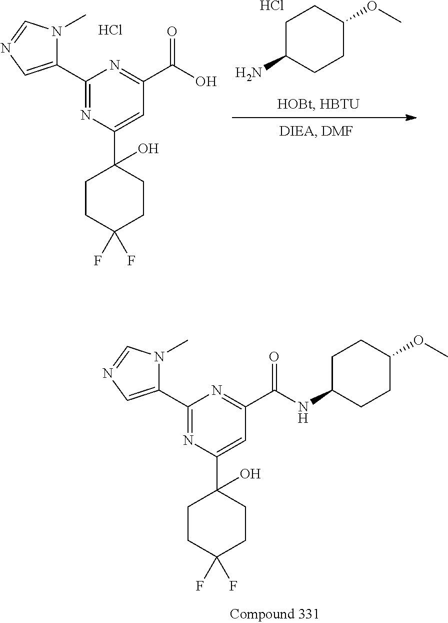

Step 3: Preparation of 6-(4,4-Difluoro-1-hydroxycyclohexyl)-N-((1r,4r)-4-methoxycyclohexyl)-2-(1-methyl-1H-imidazol-5-yl)pyrimidine-4-carboxamide. To a solution of 6-(4,4-difluoro-1-hydroxycyclohexyl)-2-(1-methyl-1H-imidazol-5-yl)pyrimidine-4-carboxylic acid hydrochloride (71 mg, 0.19 mmol) and DIEA (0.13 mL, 0.76 mmol) in DMF (1 mL) was added (1r,4r)-4-methoxycyclohexan-1-amine hydrochloride (94 mg, 0.57 mmol), HOBt (58 mg, 0.38 mmol) and HBTU (144 mg, 0.38 mmol). The reaction was stirred at r.t. overnight, diluted with water (10 mL), saturated sodium bicarbonate (5 mL), DCM (10 mL), and extracted with DCM (2×20 mL). The combined organic layers were dried over sodium sulfate, concentrated, and purified by reverse phase prep HPLC (Phenomenex Gemini 5 micron C18 Axia pack 150×21.2 mm column) using a gradient of 3-40% water/acetonitrile with 0.1% formic acid to yield 6-(4,4-difluoro-1-hydroxycyclohexyl)-N-((1r,4r)-4-methoxycyclohexyl)-2-(1-methyl-1H-imidazol-5-yl)pyrimidine-4-carboxamide (19 mg, 0.04 mmol, 22%) as an off white solid. LRMS (ESI) m/z 450.0 (M+H). ¹H NMR (400 MHz, DMSO-d₆) δ 8.54 (d, J=8.4 Hz, 1H), 8.09 (s, 1H), 8.05 (s, 1H), 7.86 (s, 1H), 5.81 (s, 1H), 4.03 (s, 3H), 3.88-3.77 (m, 1H), 3.25 (s, 3H), 3.16-3.09 (m, 1H), 2.29-2.11 (m, 4H), 2.08-1.95 (m, 4H), 1.81 (dd, J=32.1, 11.3 Hz, 4H), 1.61-1.49 (m, 2H), 1.30-1.18 (m, 2H).

Compounds 193, 194, 322, 325, 328, 329, and 335 were prepared using the methods provided in the table below.

| Compound | |
|---|---|
| Number | Method of Preparation |
| 193 | Prepared in same fashion as Compound 195 |
| 194 | Prepared in same fashion as Compound 195 |
| 322 | Synthesized in the same fashion as Compound 287 with |
| | amide bond formation at r.t. for 18 h as described for |
| | Compound 282 |
| 325 | Beginning with 2,5-dichloropyrimidine-4-carboxylic acid, |
| | amide bond formation performed in same fashion as |
| | Compound 189 and Suzuki coupling performed in the same |
| | fashion as Compound 321 |
| 328 | Beginning with 2-chloro-5-methylpyrimidine-4-carboxylic |
| | acid and (1r,4r)-4-methoxycyclohexan-1-amine HCl, amide |
| | bond formation performed in same fashion as Compound |
| | 189 and Suzuki coupling performed in the same fashion as |
| | Compound 321. |
| 329 | Beginning with 2,5-dichloropyrimidine-4-carboxylic acid, |
| | amide bond formation performed in same fashion as |
| | Compound 189 and Suzuki coupling performed in the same |
| | fashion as Compound 321. |
| 335 | Beginning with 2-chloro-5-methylpyrimidine-4-carboxylic |
| | acid and (1r,4r)-4-methoxycyclohexan-1-amine HCl, amide |
| | bond formation performed in same fashion as Compound |
| | 189 and Suzuki coupling performed in the same fashion as |
| | Compound 321. |

### Example BF

### Synthesis of Compound 321

### Preparation of 5-methyl-2-(3-methylimidazol-4-ye-N-[(1r,4r)-4-hydroxycyclohexyl]pyrimidine-4-carboxamide

Step 1: Preparation of 2-chloro-5-methyl-N-[(1r,4r)-4-hydroxycyclohexyl]pyrimidine-4-carboxamide: Beginning with 2-chloro-5-methylpyrimidine-4-carboxylic acid and trans-4-aminocyclohexanol, amide coupling performed in same fashion as Compound 256.

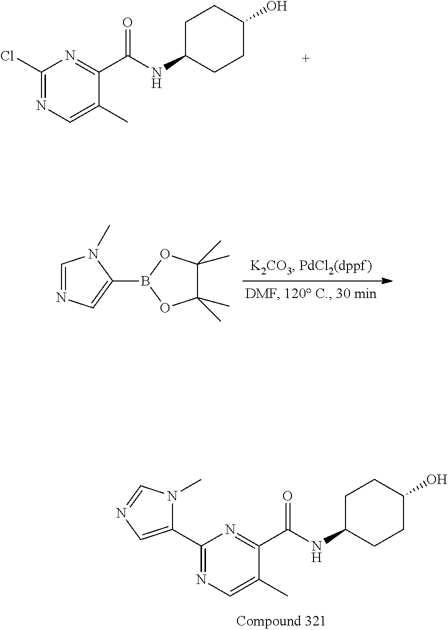 [SMILES: Cc1cnc(Cl)nc1C(=O)N[C@H]1CC[C@H](O)CC1 ; Cn1cncc1B1OC(C)(C)C(C)(C)O1 ; Cc1cnc(-c2cncn2C)nc1C(=O)N[C@H]1CC[C@H](O)CC1]

Step 2: Preparation of 5-Methyl-2-(3-methylimidazol-4-yl)-N-[(1r,4r)-4-hydroxycyclohexyl]pyrimidine-4-carboxamide. 2-chloro-5-methyl-N-[(1r,4r)-4-hydroxycyclohexyl]pyrimidine-4-carboxamide (0.257 g, 0.95 mmol) was dissolved in DMF (2 mL). 1-methyl-5-(4,4,5,5-tetramethyl-1,3,2-dioxaborolan-2-yl)imidazole (0.218 g, 1.05 mmol), potassium carbonate (0.263 g, 1.91 mmol), and [1,1′-bis(diphenylphosphino)ferrocene]dichloropalladium(II) (0.070 g, 0.095 mmol) were added and the reaction was stirred at 120° C. for 30 min. It was cooled to r.t., diluted with DCM (30 mL) and filtered through celite. The product was purified with silica gel using a gradient to 10% MeOH/DCM, followed by reverse phase HPLC using 0-100% ACN/water with formic acid over a 40 minute gradient in both phases (Phenomenex Gemini 5-micron C18 column) twice, yielding 5-methyl-2-(3-methylimidazol-4-yl)-N-[(1r,40-4-hydroxycyclohexyl]pyrimidine-4-carboxamide (0.01 g, 0.032 mmol, 3%) as a white solid. LRMS (APCI) m/z 316.0 (M+H). ¹H NMR (400 MHz, DMSO-d₆) δ 8.78 (s, 1H), 8.48-8.40 (m, 1H), 7.82 (dd, J=7.4, 3.2 Hz, 2H), 4.57 (s, 1H), 4.01 (s, 3H), 3.72 (m, 1H), 3.41 (m, 1H), 2.40 (s, 3H), 1.89-1.77 (m, 4H), 1.47-1.19 (m, 4H).

### Example BG

### Synthesis of Compound 244

### Preparation of N-(6-(difluoromethyl)pyridin-3-yl)-6-(1H-imidazol-1-yl)-4-(2-methoxyethoxy)picolinamide

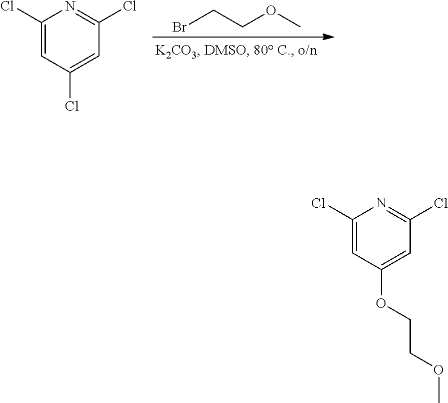

Step 1: Preparation of 2,6-Dichloro-4-(2-methoxyethoxy)pyridine: To a stirred solution of 2,6-dichloropyridin-4-ol (1.0 g, 6.10 mmol) and potassium carbonate (1.27 g, 9.15 mmol) in DMSO (10 mL) was added 2-bromoethyl methyl ether (932 mg, 6.71 mmol). The resulting mixture was stirred for 2 h at 80° C., cooled to r.t. and extracted with EtOAc (60 mL). The combined organic layers were washed twice with brine (20 mL), dried over anhydrous Na₂SO₄ and concentrated under reduced pressure to afford crude 2,6-dichloro-4-(2-methoxyethoxy)pyridine (1.3, 5.85 mmol) as a yellow oil. LRMS (ES) m/z 222 (M+H).

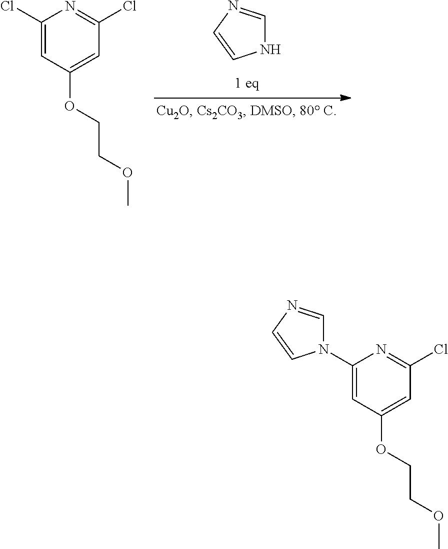 [SMILES: COCCOc1cc(Cl)nc(Cl)c1 ; c1c[nH]cn1 ; COCCOc1cc(Cl)nc(-n2ccnc2)c1]

Step 2: Preparation of 2-Chloro-6-(1H-imidazol-1-yl)-4-(2-methoxyethoxy)pyridine: Prepared using the same copper coupling conditions as Compound 210 and purified using C18 column chromatography, eluted with water (0.05% NH₄HCO₃)/MeCN (2:1) to afford 2-chloro-6-(1H-imidazol-1-yl)-4-(2-methoxyethoxy)pyridine (600 mg, 2.36 mmol, 37%) as a yellow solid. LRMS (ES) m/z 254 (M+H).

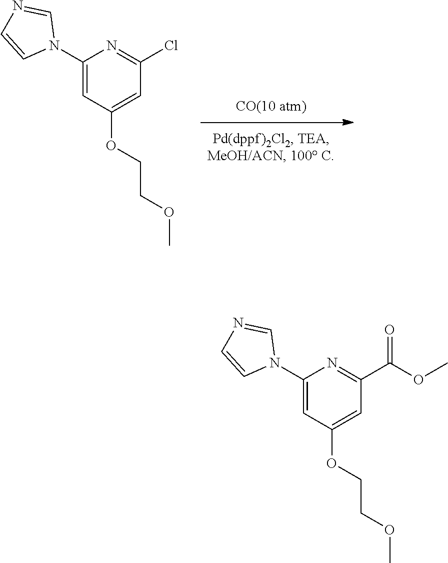 [SMILES: COCCOc1cc(Cl)nc(-n2ccnc2)c1 ; COCCOc1cc(C(=O)OC)nc(-n2ccnc2)c1]

Step 3: Preparation of Methyl 6-(1H-imidazol-1-yl)-4-(2-methoxyethoxy)picolinate: Prepared using the same carbonylation procedure as described for Compound 347 and purified by C18 column chromatography eluting with water (0.05% NH₄HCO₃)/MeCN (1:1) to afford methyl 6-(1H-imidazol-1-yl)-4-(2-methoxyethoxy)picolinate (600 mg, 2.16 mmol, 94%) as a yellow solid. LRMS (ES) m/z 278 (M+H).

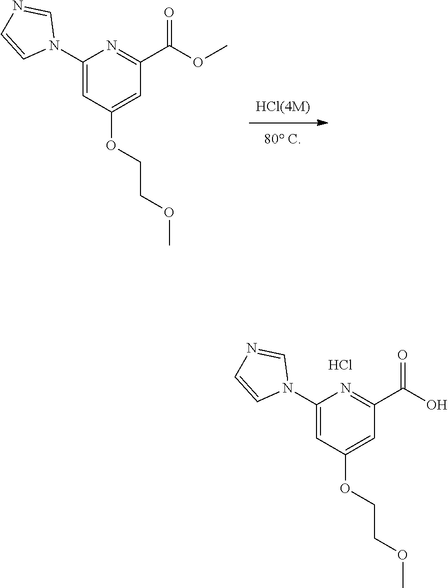 [SMILES: COCCOc1cc(C(=O)OC)nc(-n2ccnc2)c1 ; COCCOc1cc(C(=O)O)nc(-n2ccnc2)c1 ; Cl]

Step 4: Preparation of 6-(1H-Imidazol-1-yl)-4-(2-methoxyethoxy)picolinic acid HCl: A solution of methyl 6-(1H-imidazol-1-yl)-4-(2-methoxyethoxy)picolinate (580 mg, 2.09 mmol) in HCl (6 mL, 4 M) was stirred for 18 h at 80° C., cooled to r.t. and concentrated under reduced pressure to afford crude 6-(1H-imidazol-1-yl)-4-(2-methoxyethoxy)picolinic acid HCl (680 mg, 2.58 mmol) as an off-white solid. LRMS (ES) m/z 264 (M+H).

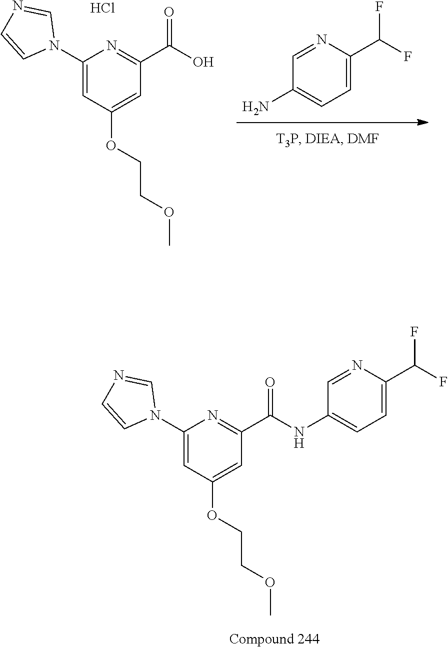 [SMILES: COCCOc1cc(C(=O)O)nc(-n2ccnc2)c1 ; Cl ; Nc1ccc(C(F)F)nc1 ; COCCOc1cc(C(=O)Nc2ccc(C(F)F)nc2)nc(-n2ccnc2)c1]

Step 5: Preparation of N-(6-(Difluoromethyl)pyridin-3-yl)-6-(1H-imidazol-1-yl)-4-(2-methoxyethoxy)picolinamide. Prepared using same amide bond coupling conditions as Compound 191 and purified by Prep-HPLC with the following conditions: (SHIMADZU HPLC) Column, XBridge Prep OBD C18 Column, 30*150 mm, 5 μm; mobile phase, water (10 mmol/L NH₄HCO₃) and ACN (33% ACN up to 63% in 7 min) to afford N-(6-(difluoromethyl)pyridin-3-yl)-6-(1H-imidazol-1-yl)-4-(2-methoxyethoxy)picolinamide (146 mg, 0.37 mmol, 74%) as a white solid. LRMS (ES) m/z 390 (M+H). ¹H NMR (300 MHz, DMSO-d₆) δ 10.80 (s, 1H), 9.21-9.12 (m, 2H), 8.50 (dd, J=8.5, 2.5 Hz, 1H), 8.42 (s, 1H), 7.83-7.70 (m, 2H), 7.66 (d, J=2.0 Hz, 1H), 7.27 (s, 1H), 6.97 (t, J=55.1 Hz, 1H), 4.49-4.40 (m, 2H), 3.80-3.71 (m, 2H), 3.33 (s, 3H).

Compounds 187, 221, 225, 226, and 245 were prepared using the methods provided in the table below.

| Compound | |
|---|---|
| Number | Method of Preparation |
| 187 | Beginning with 6-chloro-4-methoxypicolinic acid, amide |
| | bond formation using same procedure as Compound 191, |
| | followed by copper coupling as described for Compound |
| | 210. |
| 221 | Beginning with 6-chloro-4-methoxypicolinic acid, amide |
| | bond formation using same procedure as Compound 191, |
| | followed by copper coupling as described for Compound |
| | 210. |
| 225 | Beginning with 6-chloro-4-methoxypicolinic acid, amide |
| | bond formation using same procedure as Compound 191, |
| | followed by Suzuki coupling as described for Compound |
| | 347. |
| 226 | Beginning with 6-chloro-4-methoxypicolinic acid, amide |
| | bond formation using same procedure as Compound 191, |
| | followed by Suzuki coupling as described for Compound |
| | 347. |
| 245 | Prepared in same fashion as Compound 244 |

### Example BH

### Synthesis of Compound 303

### Preparation of 3-methoxy-N-((1r,4r)-4-methoxycyclohexyl)-6-(1-methyl-1H-imidazol-5-yl) pyrazine-2-carboxamide

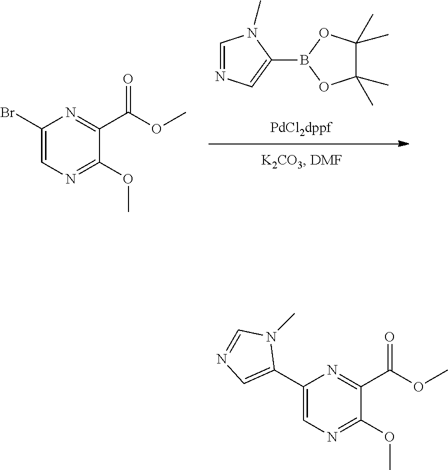 [SMILES: COC(=O)c1nc(Br)cnc1OC ; Cn1cncc1B1OC(C)(C)C(C)(C)O1 ; COC(=O)c1nc(-c2cncn2C)cnc1OC]

Step 1: Preparation of Methyl 3-methoxy-6-(1-methyl-1H-imidazol-5-yl)pyrazine-2-carboxylate. To a solution of methyl 6-bromo-3-methoxypyrazine-2-carboxylate (100 mg, 0.40 mmol) in DMF (4 mL) was added potassium carbonate (112 mg, 0.81 mmol), 1-methyl-5-(4,4,5,5-tetramethyl-1,3,2-dioxaborolan-2-yl)imidazole (93 mg, 0.45 mmol) and PdCl₂dppf (30 mg, 0.04 mmol). The resulting mixture was purged with nitrogen, heated in a sealed tube at 120° C. for 30 min, diluted with DCM, filtered through celite, concentrated under reduced pressure and purified with silica gel using 10% MeOH/DCM. The procedure was repeated beginning with additional methyl 6-bromo-3-methoxypyrazine-2-carboxylate (500 mg, 2.02 mmol) to yield methyl 3-methoxy-6-(1-methyl-1H-imidazol-5-yl)pyrazine-2-carboxylate (472 mg, 1.90 mmol, 78%) as an off-white solid which was used in the subsequent step without additional purification. LRMS (APCI) m/z 249.1 (M+H).

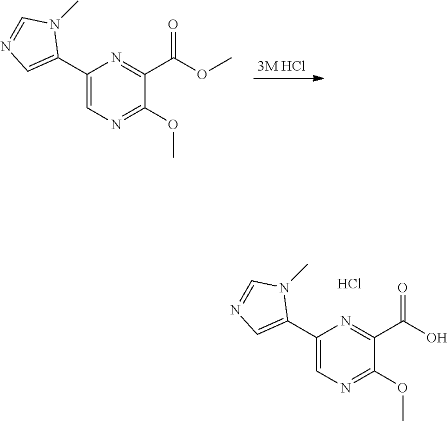 [SMILES: COC(=O)c1nc(-c2cncn2C)cnc1OC ; "3M HCl" ; COc1ncc(-c2cncn2C)nc1C(=O)O ; Cl]

Step 2: Preparation of 3-Methoxy-6-(1-methyl-1H-imidazol-5-yl)pyrazine-2-carboxylic acid hydrochloride. A solution of methyl 3-methoxy-6-(3-methylimidazol-4-yl)pyrazine-2-carboxylate (463 mg, 1.87 mmol) in 3 M hydrochloric acid (3 mL) was stirred in a sealed vial at 100° C. for 30 min. The reaction was concentrated under reduced pressure to yield 3-methoxy-6-(1-methyl-1H-imidazol-5-yl)pyrazine-2-carboxylic acid hydrochloride (406 mg, 1.87 mmol) as a tan solid which was used in subsequent steps without additional purification. LRMS (ESI) m/z 235.1 (M+H).

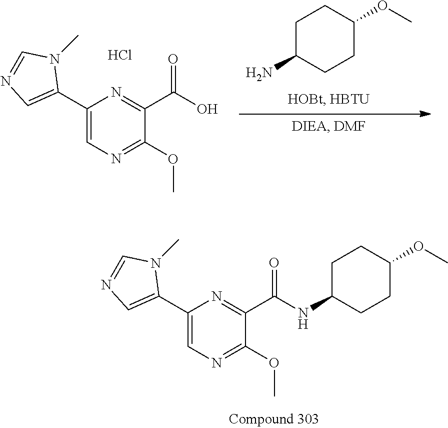

Step 3: Preparation of 3-Methoxy-N-((1r,4r)-4-methoxycyclohexyl)-6-(1-methyl-1H-imidazol-5-yl)pyrazine-2-carboxamide. To a solution of 3-methoxy-6-(1-methyl-1H-imidazol-5-yl)pyrazine-2-carboxylic acid hydrochloride (125 mg, 0.46 mmol) and DIEA (0.32 mL, 1.847 mmol) in DMF (1 mL) was added HOBt (127 mg, 0.83 mmol), HBTU (316 mg, 0.83 mmol), and (1r,4r)-4-methoxycyclohexan-1-amine (120 mg, 0.72 mmol). The resulting mixture was heated in a sealed tube at 50° C. for 5 h, diluted with water (20 mL) and extracted with DCM (2×30 mL). The combined organic layers were dried over sodium sulfate, concentrated under reduced pressure and purified with silica gel using a 0-10% MeOH/DCM gradient followed by reverse phase prep HPLC (Phenomenex Gemini 5 micron C18 Axia pack 150×21.2 mm column) using a gradient of 3-40% water/acetonitrile with 0.1% formic acid to yield 3-methoxy-N-((1r,4r)-4-methoxycyclohexyl)-6-(1-methyl-1H-imidazol-5-yl)pyrazine-2-carboxamide (20 mg, 0.06 mmol, 13%). LRMS (APCI) m/z 346.1 (M+H). ¹H NMR (400 MHz, DMSO-d₆) δ 8.66 (s, 1H), 8.34 (d, J=8.0 Hz, 1H), 7.77 (s, 1H), 7.48 (d, J=1.1 Hz, 1H), 3.95 (s, 3H), 3.85 (s, 3H), 3.77-3.68 (m, 1H), 3.23 (s, 3H), 3.17-3.08 (m, 1H), 2.04-1.95 (m, 2H), 1.92-1.83 (m, 2H). 1.39-1.17 (m, 4H).

### Example BI

### Synthesis of Compound 279

### Preparation of 3-methoxy-6-(1-methyl-1H-imidazol-5-yl)-N-(6-(trifluoromethyl)pyridin yl)pyrazine-2-carboxamide

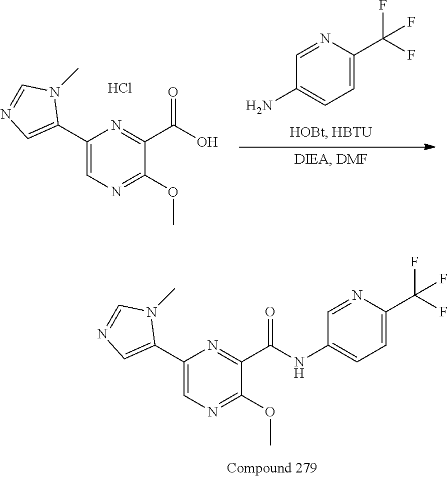

Preparation of 3-Methoxy-6-(1-methyl-1H-imidazol-5-yl)-N-(6-(trifluoromethyl)pyridin-3-yl)pyrazine-2-carboxamide. To a solution of 3-methoxy-6-(1-methyl-1H-imidazol-5-yl)pyrazine-2-carboxylic acid hydrochloride (147 mg, 0.54 mmol) and DIEA (0.38 mL, 2.17 mmol) in DMF (1 mL) was added HOBt (125 mg, 0.82 mmol), HBTU (309 mg, 0.82 mmol) and 6-(trifluoromethyl)pyridin-3-amine (133 mg, 0.82 mmol). The resulting mixture was heated in a sealed tube at 70° C. for 16 h, cooled to r.t., diluted with water (10 mL) and extracted with DCM (2×30 mL). The combined organic layers were dried over sodium sulfate, concentrated under reduced pressure and purified twice with silica using a 0-10% MeOH/DCM gradient to yield 3-methoxy-6-(1-methyl-1H-imidazol-5-yl)-N-(6-(trifluoromethyl)pyridin-3-yl)pyrazine-2-carboxamide (44 mg, 0.12 mmol, 21%) as an off-white solid. LRMS (ESI) m/z 379.1 (M+H). ¹H NMR (400 MHz, DMSO-d₆) δ 11.13 (s, 1H), 9.06 (d, J=2.4 Hz, 1H), 8.84 (s, 1H), 8.49 (dd, J=8.7, 2.4 Hz, 1H), 7.95 (d, J=8.6 Hz, 1H), 7.82 (s, 1H), 7.58 (d, J=1.1 Hz, 1H), 4.03 (s, 3H), 3.91 (s, 3H).

### Example BJ

### Synthesis of Compound 298

### Preparation of N-((1r,4r)-4-methoxycyclohexyl)-3-methyl-6-(1-methyl-1H-imidazol-5-yl) pyrazine-2-carboxamide

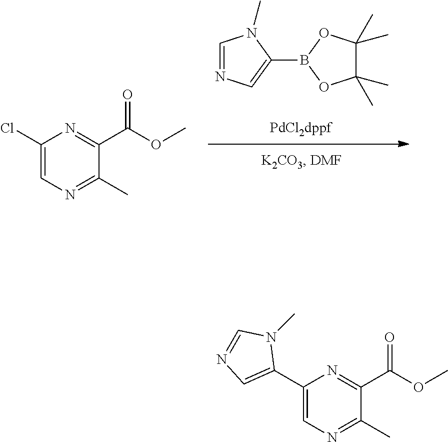 [SMILES: COC(=O)c1nc(Cl)cnc1C ; Cn1cncc1B1OC(C)(C)C(C)(C)O1 ; COC(=O)c1nc(-c2cncn2C)cnc1C]

Step 1: Preparation of Methyl 3-methyl-6-(1-methyl-1H-imidazol-5-yl)pyrazine-2-carboxylate. To a solution of methyl 6-chloro-3-methylpyrazine-2-carboxylate (400 mg, 2.14 mmol) in DMF (4 mL) was added potassium carbonate (592.5 mg, 4.29 mmol), 1-methyl-5-(4,4,5,5-tetramethyl-1,3,2-dioxaborolan-2-yl)imidazole (490.6 mg, 2.36 mmol), and PdCl₂dppf (156.8 mg, 0.21 mmol). The resulting mixture was purged with nitrogen and stirred at 120° C. in a sealed tube for 30 min, cooled to r.t., filtered through celite, and purified by silica gel chromatography using a 0-10% MeOH/DCM gradient to afford methyl 3-methyl-6-(1-methyl-1H-imidazol-5-yl)pyrazine-2-carboxylate (407 mg, 1.75 mmol, 82%) as a tan solid. The material was used in next step without further purification. LRMS (ESI) m/z 233.1 (M+H).

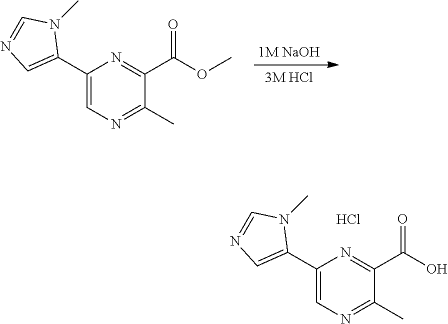

Step 2: Preparation of 3-Methyl-6-(1-methyl-1H-imidazol-5-yl)pyrazine-2-carboxylic acid hydrochloride. A solution of methyl 3-methyl-6-(1-methyl-1H-imidazol-5-yl)pyrazine-2-carboxylate (407 mg, 1.75 mmol) in 1 M sodium hydroxide (5.25 mL, 5.25 mmol) was stirred at r.t. for 10 min and directly purified by C18 column chromatography, eluting with a gradient of 0-100% water/acetonitrile with 0.1% formic acid. The product was dissolved in 3 M hydrochloric acid (1.75 mL, 5.25 mmol) and concentrated to yield 3-methyl-6-(1-methyl-1H-imidazol-5-yl)pyrazine-2-carboxylic acid hydrochloride (446 mg, 1.75 mmol, 99.9%) as an off-white solid in quantitative yield. LRMS (ESI) m/z 219.1 (M+H).

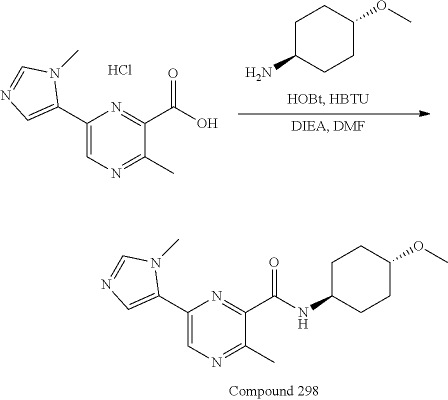

Step 3: Preparation of N-((1r,4r)-4-methoxycyclohexyl)-3-methyl-6-(1-methyl-1H-imidazol-5-yl)pyrazine-2-carboxamide. To a solution of 3-methyl-6-(1-methyl-1H-imidazol-5-yl)pyrazine-2-carboxylic acid hydrochloride (117 mg, 0.46 mmol) and DIEA (0.32 mL, 1.83 mmol) in DMF (1 mL) was added HOBt (105 mg, 0.69 mmol), HBTU (261 mg, 0.69 mmol), and (1r,4r)-4-methoxycyclohexan-1-amine (84 mg, 0.50 mmol). The resulting mixture was stirred at r.t. for 18 h, diluted with water (5 mL), saturated sodium bicarbonate (20 mL) and extracted with DCM (2×30 mL). The combined organic layers were dried over sodium sulfate, concentrated, and purified by silica gel chromatography using a 0-10% MeOH/DCM gradient followed by reverse phase prep HPLC (Phenomenex Gemini 5 micron C18 Axia pack 150×21.2 mm column) using a gradient of 3-40% water/acetonitrile with 0.1% formic acid to yield N-((1r,4r)-4-methoxycyclohexyl)-3-methyl-6-(1-methyl-1H-imidazol-5-yl)pyrazine-2-carboxamide (12 mg, 0.04 mmol, 8% yield) as a white solid. LRMS (ESI) m/z 330.0 (M+H). ¹H NMR (400 MHz, DMSO-d₆) δ 9.02 (s, 1H), 8.38 (d, J=7.4 Hz, 1H), 7.84 (s, 1H), 7.73 (s, 1H), 3.95 (s, 3H), 3.83-3.71 (m, 1H), 3.24 (s, 3H), 3.19-3.08 (m, 1H), 2.67 (s, 3H), 2.01 (d, J=12.4 Hz, 2H), 1.88 (d, J=11.7 Hz, 2H), 1.45-1.32 (m, 2H), 1.30-1.18 (m, 2H).

### Example BK

### Synthesis of Compound 296

### Preparation of 3-methyl-6-(1-methyl-1H-imidazol-5-yl)-N-(6-(trifluoromethyl)pyridin-3-yl)pyrazine-2-carboxamide

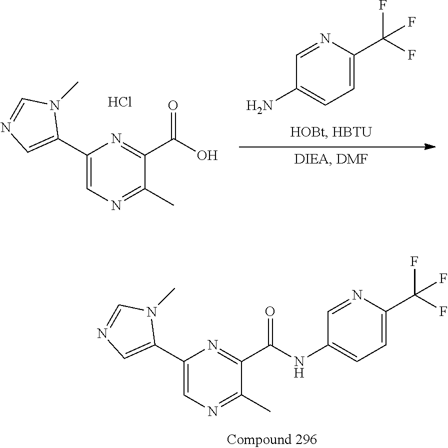

Preparation of 3-Methyl-6-(1-methyl-1H-imidazol-5-yl)-N-(6-(trifluoromethyl)pyridin-3-yl)pyrazine-2-carboxamide. To a solution of 3-methyl-6-(1-methyl-1H-imidazol-5-yl)pyrazine-2-carboxylic acid hydrochloride (100 mg, 0.39 mmol) and DIEA (0.27 mL, 1.57 mmol) in DMF (1 mL) was added HOBt (90 mg, 0.59 mmol), HBTU (223 mg, 0.59 mmol) and 6-(trifluoromethyl)pyridin-3-amine (127 mg, 0.79 mmol). The resulting mixture was heated in a sealed tube at 80° C. for 3 h, cooled to r.t., filtered and purified by reverse phase prep HPLC (Phenomenex Gemini 5 micron C18 Axia pack 150×21.2 mm column) using a gradient of 3-40% water/acetonitrile with 0.1% formic acid followed by silica gel chromatography using a 0-10% MeOH/DCM gradient to yield 3-methyl-6-(1-methyl-1H-imidazol-5-yl)-N-(6-(trifluoromethyl)pyridin-3-yl)pyrazine-2-carboxamide (29 mg, 0.08 mmol, 20%) as a white solid. LRMS (ESI) m/z 363.0 (M+H). ¹H NMR (400 MHz, DMSO-d₆) δ 11.07 (s, 1H), 9.16 (s, 1H), 9.12 (s, 1H), 8.55 (d, J=8.5 Hz, 1H), 7.96 (d, J=8.6 Hz, 1H), 7.90 (s, 1H), 7.82 (s, 1H), 4.02 (s, 3H), 2.79 (s, 3H).

### Example BL

### Synthesis of Compound 318

### Preparation of 3-amino-N-((1r,4r)-4-methoxycyclohexyl)-6-(1-methyl-1H-imidazol-5-yl)pyrazine-2-carboxamide

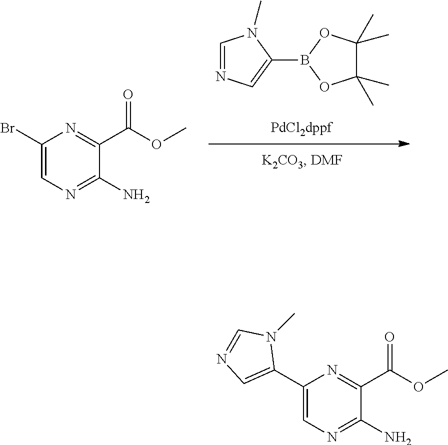 [SMILES: COC(=O)c1nc(Br)cnc1N ; Cn1cncc1B1OC(C)(C)C(C)(C)O1 ; COC(=O)c1nc(-c2cncn2C)cnc1N]

Step 1: Preparation of Methyl 3-amino-6-(1-methyl-1H-imidazol-5-yl)pyrazine-2-carboxylate. To a solution of methyl 3-amino-6-bromopyrazine-2-carboxylate (500 mg, 2.16 mmol) in DMF (5 mL) was added potassium carbonate (596 mg, 4.31 mmol), PdCl₂dppf (158 mg, 0.22 mmol), and 1-methyl-5-(4,4,5,5-tetramethyl-1,3,2-dioxaborolan-2-yl)imidazole (493 mg, 2.37 mmol). The resulting mixture was heated at 120° C. for 30 min, cooled to r.t., diluted with DCM (20 mL), filtered through celite, concentrated and purified by silica gel chromatography using a 0-10% MeOH/DCM gradient to yield methyl 3-amino-6-(1-methyl-1H-imidazol-5-yl)pyrazine-2-carboxylate (502 mg, 2.15 mmol, 99.9%) as a tan solid.

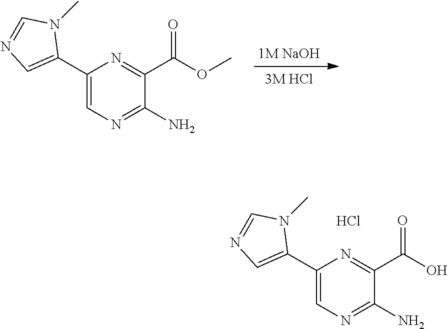

Step 2: Preparation of 3-Amino-6-(1-methyl-1H-imidazol-5-yl)pyrazine-2-carboxylic acid hydrochloride. A solution of methyl 3-amino-6-(3-methylimidazol-4-yl)pyrazine-2-carboxylate (360 mg, 1.54 mmol) and 1 M sodium hydroxide (4.6 mL, 4.63 mmol) was stirred at r.t. for 20 min, concentrated under reduced pressure, acidified with 3 M hydrochloric acid (2.05 mL, 6.17 mmol) and concentrated to yield 3-amino-6-(1-methyl-1H-imidazol-5-yl)pyrazine-2-carboxylic acid hydrochloride (338 mg, 1.54 mmol, 99.9%) as a tan solid in quantitative yield. LRMS (ESI) m/z 220.0 (M+H).

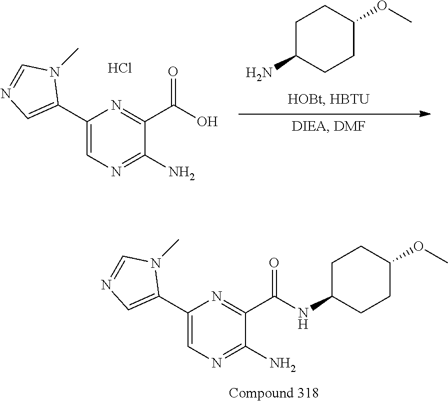

Step 3: Preparation of 3-Amino-N-((1r,4r)-4-methoxycyclohexyl)-6-(1-methyl-1H-imidazol-5-yl)pyrazine-2-carboxamide. To a solution of 3-amino-6-(1-methyl-1H-imidazol-5-yl)pyrazine-2-carboxylic acid hydrochloride (150 mg, 0.68 mmol) and DIEA (0.48 mL, 2.74 mmol) in DMF (3 mL) was added (1r,4r)-4-methoxycyclohexan-1-amine (114.9 mg, 0.89 mmol), HOBt (138.7 mg, 1.03 mmol), and HBTU (389.3 mg, 1.02 mmol). The resulting mixture was stirred at r.t. for 18 h, filtered and purified by reverse phase prep HPLC (Phenomenex Gemini 5 micron C18 Axia pack 150×21.2 mm column) using a gradient of 3-40% water/acetonitrile with 0.1% formic acid followed by silica gel chromatography using a 0-10% MeOH/DCM gradient followed by revers phase HPLC using the same conditions above to yield 3-amino-N-((1r,4r)-4-methoxycyclohexyl)-6-(1-methyl-1H-imidazol-5-yl)pyrazine-2-carboxamide (39 mg, 0.12 mmol, 17%) as a white solid. LRMS (ESI) m/z 331.1 (M+H). ¹H NMR (400 MHz, DMSO-d₆) δ 8.54 (s, 1H), 8.15 (s, 1H), 7.73 (s, 1H), 7.59 (s, 2H), 7.39 (s, 1H), 3.84 (s, 3H), 3.80-3.69 (m, 1H), 3.23 (s, 3H), 3.10 (t, J=11.7 Hz, 1H), 2.00 (d, J=12.5 Hz, 2H), 1.84 (d, J=12.6 Hz, 2H), 1.46 (q, J=12.5 Hz, 2H), 1.24 (q, J=12.1 Hz, 2H).

### Example BM

### Synthesis of Compound 314

### Preparation of 3-amino-6-(1-methyl-1H-imidazol-5-yl)-N-(6-(trifluoromethyl)pyridin yl)pyrazine-2-carboxamide

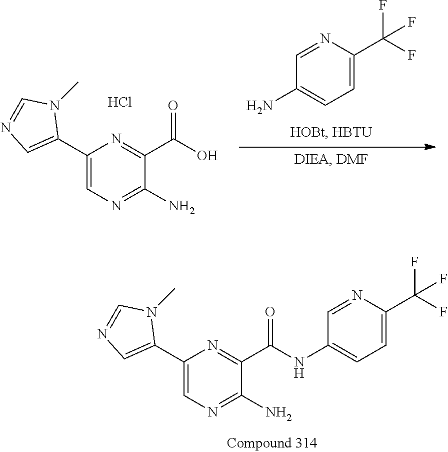

Preparation of 3-Amino-6-(1-methyl-1H-imidazol-5-yl)-N-(6-(trifluoromethyl)pyridin-3-yl)pyrazine-2-carboxamide. To a solution of 3-amino-6-(1-methyl-1H-imidazol-5-yl)pyrazine-2-carboxylic acid hydrochloride (150 mg, 0.68 mmol) and DIEA (0.48 mL, 2.73 mmol) in DMF (1.5 mL) was added 6-(trifluoromethyl)pyridin-3-amine (222 mg, 1.37 mmol), HOBt (139 mg, 1.02 mmol) and HBTU (389 mg, 1.02 mmol). The resulting mixture was stirred at 80° C. for 18 h, cooled to r.t., filtered and purified by reverse phase prep HPLC (Phenomenex Gemini 5 micron C18 Axia pack 150×21.2 mm column) using a gradient of 3-40% water/acetonitrile with 0.1% formic acid to yield 3-amino-6-(1-methyl-1H-imidazol-5-yl)-N-(6-(trifluoromethyl)pyridin-3-yl)pyrazine-2-carboxamide (14 mg, 0.04 mmol, 6%) as a white solid. LRMS (APCI) m/z 364.4 (M+H). ¹H NMR (400 MHz, DMSO-d₆) δ 10.79 (s, 1H), 9.15 (d, J=6.5 Hz, 2H), 8.72 (d, J=2.1 Hz, 1H), 8.54 (d, J=8.9 Hz, 1H), 8.17 (s, 1H), 8.01-7.94 (m, 3H), 4.10 (s, 3H).

### Example BN

### Synthesis of Compound 357

### Preparation of N-(4-(2-hydroxypropan-2-yl)phenyl)-4-(1H-imidazol-1-yl)pyrimidine-2-carboxamide

Synthesized in the same fashion as compound 249 to provide N-(4-(2-hydroxypropan-2-yl)phenyl)-4-(1H-imidazol-1-yl)pyrimidine-2-carboxamide (143 mg, 0.44 mmol, 37% yield) as a white solid. LRMS (APCI) m/z 324.1 (M+H). ¹H NMR (400 MHz, DMSO-d₆) δ 10.65 (s, 1H), 9.12 (d, J=5.5 Hz, 1H), 8.97 (s, 1H), 8.28 (s, 1H), 8.12 (d, J=5.6 Hz, 1H), 7.77 (d, J=8.2 Hz, 2H), 7.48 (d, J=8.2 Hz, 2H), 7.24 (s, 1H), 4.99 (s, 1H), 1.44 (s, 6H).

### Example BO

### Synthesis of Compound 357

### Preparation of N-(4-(2-hydroxypropan-2-yl)phenyl)-4-(1-methyl-1H-imidazol-5-yl)pyrimidine-2-carboxamide

Synthesized in the same fashion as compound 250 to provide N-(4-(2-hydroxypropan-2-yl)phenyl)-4-(1-methyl-1H-imidazol-5-yl)pyrimidine-2-carboxamide (90 mg, 0.27 mmol, 22% yield) as a white solid. LRMS (APCI) m/z 338.1 (M+H). ¹H NMR (400 MHz, DMSO-d₆) δ 10.54 (s, 1H), 8.91 (d, J=5.2 Hz, 1H), 8.08-7.88 (m, 4H), 7.77 (d, J=8.2 Hz, 2H), 7.46 (d, J=8.2 Hz, 2H), 4.98 (s, 1H), 4.13 (s, 3H), 1.43 (s, 6H).

The following compounds were prepared in accordance with the synthetic procedures described herein or using similar synthetic procedures with the appropriate reagents.

| Compound | | Compound | |
|---|---|---|---|
| Number | Analysis | Number | Analysis |
| 1 | ESI m/z 266.1 [M + H]+ | 2 | APCI m/z 280.1 [M + H]+ |
| 3 | APCI m/z 284.1 [M + H]+ | 4 | APCI m/z 266.1 [M + H]+ |
| 5 | APCI m/z 265.1 [M + H]+ | 6 | APCI m/z 334.1 [M + H]+ |
| 7 | APCI m/z 266.1 [M + H]+ | 8 | APCI m/z 266.1 [M + H]+ |
| 9 | APCI m/z 265.1 [M + H]+ | 10 | APCI m/z 266.2 [M + H]+ |
| 11 | APCI m/z 283.0 [M + H]+ | 12 | APCI m/z 273.1 [M + H]+ |
| 13 | APCI m/z 334.0 [M + H]+ | 14 | APCI m/z 345.1 [M + H]+ |
| 15 | APCI m/z 271.1 [M + H]+ | 16 | APCI m/z 255.1 [M + H]+ |
| 17 | APCI m/z 283.1 [M + H]+ | 18 | APCI m/z 283.1 [M + H]+ |
| 19 | APCI m/z 283.0 [M + H]+ | 20 | APCI m/z 280.0 [M + H]+ |
| 21 | APCI m/z 280.0 [M + H]+ | 22 | APCI m/z 280.1 [M + H]+ |
| 23 | APCI m/z 273.1 [M + H]+ | 24 | APCI m/z 301.0 [M + H]+ |
| 25 | APCI m/z 296.1 [M + H]+ | 26 | APCI m/z 280.1 [M + H]+ |
| 27 | APCI m/z 280.1 [M + H]+ | 28 | APCI m/z 301.1 [M + H]+ |
| 29 | APCI m/z 348.1 [M + H]+ | 30 | APCI m/z 287.1 [M + H]+ |
| 31 | APCI m/z 287.1 [M + H]+ | 32 | APCI m/z 280.1 [M + H]+ |
| 33 | APCI m/z 280.1 [M + H]+ | 34 | APCI m/z 301.0 [M + H]+ |
| 35 | APCI m/z 297.0 [M + H]+ | 36 | APCI m/z 267.1 [M + H]+ |
| 37 | APCI m/z 267.0 [M + H]+ | 38 | APCI m/z 334.1 [M + H]+ |
| 39 | APCI m/z 316.0 [M + H]+ | 40 | APCI m/z 344.0 [M + H]+ |
| 41 | APCI m/z 351.0 [M + H]+ | 42 | APCI m/z 352.0 [M + H]+ |
| 43 | APCI m/z 363.1 [M + H]+ | 44 | ESI m/z 314 [M + H]+ |
| 45 | ESI m/z 330 [M + H]+ | 46 | ESI m/z 301 [M + H]+ |
| 47 | ESI m/z 326 [M + H]+ | 48 | ESI m/z 342 [M + H]+ |
| 49 | ESI m/z 273 [M + H]+ | 50 | ESI m/z 351 [M + H]+ |
| 51 | ESI m/z 297 [M + H]+ | 52 | ESI m/z 297 [M + H]+ |
| 53 | APCI m/z 352.1 [M + H]+ | 54 | APCI m/z 363.2 [M + H]+ |
| 55 | APCI m/z 335.0 [M + H]+ | 56 | APCI m/z 267.1 [M + H]+ |
| 57 | APCI m/z 287.2 [M + H]+ | 58 | APCI m/z 389.1 [M + H]+ |
| 59 | APCI m/z 280.1 [M + H]+ | 60 | APCI m/z 369.1 [M + H]+ |
| 61 | APCI m/z 413.1 [M + H]+ | 62 | APCI m/z 355.1 [M + H]+ |
| 63 | APCI m/z 259.1 [M + H]+ | 64 | APCI m/z 321.1 [M + H]+ |
| 65 | APCI m/z 359.2 [M + H]+ | 66 | APCI m/z 377.0 [M + H]+ |
| 67 | APCI m/z 335.0 [M + H]+ | 68 | APCI m/z 322.1 [M + H]+ |
| 69 | APCI m/z 349.1 [M + H]+ | 70 | APCI m/z 336.1 [M + H]+ |
| 71 | APCI m/z 348.1 [M + H]+ | 72 | APCI m/z 335.1 [M + H]+ |
| 73 | APCI m/z 322.1 [M + H]+ | 74 | APCI m/z 349.0 [M + H]+ |
| 75 | APCI m/z 336.1 [M + H]+ | 76 | APCI m/z 377.0 [M + H]+ |
| 77 | APCI m/z 335.1 [M + H]+ | 78 | APCI m/z 320.1 [M + H]+ |
| 79 | APCI m/z 340.1 [M + H]+ | 80 | APCI m/z 302.1 [M + H]+ |
| 81 | APCI m/z 286.1 [M + H]+ | 82 | APCI m/z 334.1 [M + H]+ |
| 83 | APCI m/z 354.1 [M + H]+ | 84 | APCI m/z 316.1 [M + H]+ |
| 85 | APCI m/z 300.2 [M + H]+ | 86 | APCI m/z 352.0 [M + H]+ |
| 87 | APCI m/z 339.1 [M + H]+ | 88 | APCI m/z 302.1 [M + H]+ |
| 89 | APCI m/z 322.1 [M + H]+ | 90 | APCI m/z 349.1 [M + H]+ |
| 91 | APCI m/z 316.1 [M + H]+ | 92 | APCI m/z 336.1 [M + H]+ |
| 93 | APCI m/z 317.0 [M + H]+ | 94 | APCI m/z 331.0 [M + H]+ |
| 95 | APCI m/z 316.1 [M + H]+ | 96 | APCI m/z 315.1 [M + H]+ |
| 97 | APCI m/z 335.1 [M + H]+ | 98 | APCI m/z 329.1 [M + H]+ |
| 99 | APCI m/z 349.1 [M + H]+ | 100 | APCI m/z 315.1 [M + H]+ |
| 101 | APCI m/z 335.1 [M + H]+ | 102 | APCI m/z 286.1 [M + H]+ |
| 103 | APCI m/z 320.1 [M + H]+ | 104 | APCI m/z 300.1 [M + H]+ |
| 105 | APCI m/z 334.1 [M + H]+ | 106 | APCI m/z 330.1 [M + H]+ |
| 107 | APCI m/z 344.1 [M + H]+ | 108 | APCI m/z 359.1 [M + H]+ |
| 109 | APCI m/z 392.1 [M + H]+ | 110 | APCI m/z 374.0 [M + H]+ |
| 111 | APCI m/z 370.1 [M + H]+ | 112 | APCI m/z 303.1 [M + H]+ |
| 113 | APCI m/z 319.1 [M + H]+ | 114 | APCI m/z 334.0 [M + H]+ |
| 115 | APCI m/z 420.0 [M + H]+ | 116 | APCI m/z 402.0 [M + H]+ |
| 117 | ESI m/z 349 [M + H]+ | 118 | ESI m/z 316 [M + H]+ |
| 119 | ESI m/z 300 [M + H]+ | 120 | ESI m/z 336 [M + H]+ |
| 121 | ESI m/z 330 [M + H]+ | 122 | ESI m/z 350 [M + H]+ |
| 123 | ESI m/z 314 [M + H]+ | 124 | ESI m/z 348 [M + H]+ |
| 125 | APCI m/z 332.1 [M + H]+ | 126 | APCI m/z 318.1 [M + H]+ |
| 127 | APCI m/z 347.0 [M + H]+ | 128 | APCI m/z 346.1 [M + H]+ |
| 129 | APCI m/z 361.0 [M + H]+ | 130 | APCI m/z 349.1 [M + H]+ |
| 131 | APCI m/z 364.0 [M + H]+ | 132 | APCI m/z 347.0 [M + H]+ |
| 133 | APCI m/z 332.1 [M + H]+ | 134 | APCI m/z 329.1 [M + H]+ |
| 135 | APCI m/z 305.1 [M + H]+ | 136 | APCI m/z 307.1 [M + H]+ |
| 137 | ESI m/z 349 [M + H]+ | 138 | ESI m/z 390 [M + H]+ |
| 139 | APCI m/z 333.1 [M + H]+ | 140 | APCI m/z 319.1 [M + H]+ |
| 141 | APCI m/z 359.1 [M + H]+ | 142 | APCI m/z 379.0 [M + H]+ |
| 143 | APCI m/z 335.0 [M + H]+ | 144 | APCI m/z 317.0 [M + H]+ |
| 145 | APCI m/z 302.1 [M + H]+ | 146 | APCI m/z 286.1 [M + H]+ |
| 147 | APCI m/z 331.1 [M + H]+ | 148 | APCI m/z 316.1 [M + H]+ |
| 149 | APCI m/z 300.1 [M + H]+ | 150 | APCI m/z 334.0 [M + H]+ |
| 151 | ESI m/z 384 [M + H]+ | 152 | ESI m/z 404 [M + H]+ |
| 153 | ESI m/z 368 [M + H]+ | 154 | ESI m/z 402 [M + H]+ |
| 155 | ESI m/z 374 [M + H]+ | 156 | ESI m/z 360 [M + H]+ |
| 157 | ESI m/z 414 [M + H]+ | 158 | APCI m/z 375.1 [M + H]+ |
| 159 | APCI m/z 389.1 [M + H]+ | 160 | APCI m/z 342.2 [M + H]+ |
| 161 | APCI m/z 362.1 [M + H]+ | 162 | APCI m/z 357.1 [M + H]+ |
| 163 | ESI m/z 363 [M + H]+ | 164 | ESI m/z 354 [M + H]+ |
| 165 | ESI m/z 428 [M + H]+ | 166 | ESI m/z 315 [M + H]+ |
| 167 | APCI m/z 344.2 [M + H]+ | 168 | APCI m/z 364.1 [M + H]+ |
| 169 | APCI m/z 359.1 [M + H]+ | 170 | APCI m/z 377.1 [M + H]+ |
| 171 | APCI m/z 358.2 [M + H]+ | 172 | APCI m/z 378.2 [M + H]+ |
| 173 | APCI m/z 373.1 [M + H]+ | 174 | APCI m/z 391.1 [M + H]+ |
| 175 | APCI m/z 393.1 [M + H]+ | 176 | APCI m/z 407.1 [M + H]+ |
| 177 | APCI m/z 394.1 [M + H]+ | 178 | APCI m/z 374.2 [M + H]+ |
| 179 | ESI m/z 402 [M + H]+ | 180 | ESI m/z 370 [M + H]+ |
| 181 | APCI m/z 370.2 [M + H]+ | 182 | APCI m/z 376.2 [M + H]+ |
| 183 | APCI m/z 385.0 [M + H]+ | 184 | APCI m/z 356.1 [M + H]+ |
| 185 | APCI m/z 362.2 [M + H]+ | 186 | APCI m/z 371.1 [M + H]+ |
| 187 | ESI m/z 346 [M + H]+ | 188 | APCI m/z 373.2 [M + H]+ |
| 189 | APCI m/z 387.1 [M + H]+ | 190 | APCI m/z 405.1 [M + H]+ |
| 191 | ESI m/z 330 [M + H]+ | 192 | ESI m/z 346 [M + H]+ |
| 193 | APCI m/z 386.2 [M + H]+ | 194 | APCI m/z 392.1 [M + H]+ |
| 195 | APCI m/z 401.1 [M + H]+ | 196 | APCI m/z 419.1 [M + H]+ |
| 197 | APCI m/z 415.1 [M + H]+ | 198 | APCI m/z 433.1 [M + H]+ |
| 199 | APCI m/z 400.2 [M + H]+ | 200 | APCI m/z 406.1 [M + H]+ |
| 201 | ESI m/z 361 [M + H]+ | 202 | ESI m/z 345 [M + H]+ |
| 203 | ESI m/z 356 [M + H]+ | 204 | ESI m/z 342 [M + H]+ |
| 205 | ESI m/z 357 [M + H]+ | 206 | ESI m/z 371 [M + H]+ |
| 207 | ESI m/z 316 [M + H]+ | 208 | ESI m/z 332 [M + H]+ |
| 209 | APCI m/z 360.1 [M + H]+ | 210 | ESI m/z 347 [M + H]+ |
| 211 | APCI m/z 320.1 [M + H]+ | 212 | APCI m/z 320.1 [M + H]+ |
| 213 | APCI m/z 334.1 [M + H]+ | 214 | APCI m/z 334.1 [M + H]+ |
| 215 | ESI m/z 331 [M + H]+ | 216 | ESI m/z 333 [M + H]+ |
| 217 | APCI m/z 350.1 [M + H]+ | 218 | APCI m/z 364.1 [M + H]+ |
| 219 | APCI m/z 336.1 [M + H]+ | 220 | APCI m/z 350.1 [M + H]+ |
| 221 | ESI m/z 364 [M + H]+ | 222 | APCI m/z 332.1 [M + H]+ |
| 223 | APCI m/z 318.1 [M + H]+ | 224 | ESI m/z 405 [M + H]+ |
| 225 | ESI m/z 360 [M + H]+ | 226 | ESI m/z 378 [M + H]+ |
| 227 | APCI m/z 420.2 [M + H]+ | 228 | APCI m/z 422.1 [M + H]+ |
| 229 | APCI m/z 422.1 [M + H]+ | 230 | APCI m/z 418.1 [M + H]+ |
| 231 | ESI m/z 390 [M + H]+ | 232 | ESI m/z 374 [M + H]+ |
| 233 | ESI m/z 423 [M + H]+ | 234 | ESI m/z 379 [M + H]+ |
| 235 | ESI m/z 363 [M + H]+ | 236 | APCI m/z 338.1 [M + H]+ |
| 237 | APCI m/z 338.1 [M + H]+ | 238 | APCI m/z 338.1 [M + H]+ |
| 239 | APCI m/z 356.1 [M + H]+ | 240 | APCI m/z 352.1 [M + H]+ |
| 241 | APCI m/z 352.1 [M + H]+ | 242 | APCI m/z 352.1 [M + H]+ |
| 243 | APCI m/z 370.1 [M + H]+ | 244 | ESI m/z 390 [M + H]+ |
| 245 | ESI m/z 408 [M + H]+ | 246 | APCI m/z 349.2 [M + H]+ |
| 247 | APCI m/z 335.1 [M + H]+ | 248 | APCI m/z 325.1 [M + H]+ |
| 249 | APCI m/z 325.1 [M + H]+ | 250 | APCI m/z 339.1 [M + H]+ |
| 251 | APCI m/z 330.2 [M + H]+ | 252 | APCI m/z 378.2 [M + H]+ |
| 253 | APCI m/z 378.2 [M + H]+ | 254 | APCI m/z 344.2 [M + H]+ |
| 255 | APCI m/z 330.2 [M + H]+ | 256 | APCI m/z 302.1 [M + H]+ |
| 257 | APCI m/z 288.1 [M + H]+ | 258 | APCI m/z 316.1 [M + H]+ |
| 259 | APCI m/z 316.1 [M + H]+ | 260 | APCI m/z 364.1 [M + H]+ |
| 261 | APCI m/z 302.2 [M + H]+ | 262 | APCI m/z 321.2 [M + H]+ |
| 263 | APCI m/z 349.2 [M + H]+ | 264 | APCI m/z 383.1 [M + H]+ |
| 265 | APCI m/z 369.1 [M + H]+ | 266 | APCI m/z 403 [M + H]+ |
| 267 | APCI m/z 423 [M + H]+ | 268 | APCI m/z 389 [M + H]+ |
| 269 | APCI m/z 389 [M + H]+ | 270 | APCI m/z 403 [M + H]+ |
| 271 | APCI m/z 389 [M + H]+ | 272 | APCI m/z 389 [M + H]+ |
| 273 | APCI m/z 403 [M + H]+ | 274 | APCI m/z 403 [M + H]+ |
| 275 | APCI m/z 352.1 [M + H]+ | 276 | APCI m/z 347.1 [M + H]+ |
| 277 | APCI m/z 434.2 [M + H]+ | 278 | APCI m/z 316.2 [M + H]+ |
| 279 | APCI m/z 379.1 [M + H]+ | 280 | APCI m/z 404.2 [M + H]+ |
| 281 | APCI m/z 436.2 [M + H]+ | 282 | APCI m/z 431.1 [M + H]+ |
| 283 | APCI m/z 400.2 [M + H]+ | 284 | APCI m/z 316.2 [M + H]+ |
| 285 | APCI m/z 467 [M + H]+ | 286 | APCI m/z 418 [M + H]+ |
| 287 | APCI m/z 449 [M + H]+ | 288 | APCI m/z 420 [M + H]+ |
| 289 | APCI m/z 434 [M + H]+ | 290 | APCI m/z 286 [M + H]+ |
| 291 | APCI m/z 434.2 [M + H]+ | 292 | APCI m/z 412.1 [M + H]+ |
| 293 | APCI m/z 338.0 [M + H]+ | 294 | APCI m/z 328.1 [M + H]+ |
| 295 | APCI m/z 386.2 [M + H]+ | 296 | APCI m/z 363.0 [M + H]+ |
| 297 | APCI m/z 288.0 [M + H]+ | 298 | APCI m/z 330.0 [M + H]+ |
| 299 | APCI m/z 302.1 [M + H]+ | 300 | APCI m/z 467.0 [M + H]+ |
| 301 | APCI m/z 288.1 [M + H]+ | 302 | APCI m/z 288.0 [M + H]+ |
| 303 | APCI m/z 346.1 [M + H]+ | 304 | APCI m/z 274.0 [M + H]+ |
| 305 | APCI m/z 288.1 [M + H]+ | 306 | APCI m/z 300 [M + H]+ |
| 307 | APCI m/z 405 [M + H]+ | 308 | APCI m/z 356.1 [M + H]+ |
| 309 | APCI m/z 419.1 [M + H]+ | 310 | APCI m/z 350.1 [M + H]+ |
| 311 | APCI m/z 422.2 [M + H]+ | 312 | APCI m/z 392.4 [M + H]+ |
| 313 | APCI m/z 274.0 [M + H]+ | 314 | APCI m/z 364.4 [M + H]+ |
| 315 | APCI m/z 428.4 [M + H]+ | 316 | APCI m/z 428.4 [M + H]+ |
| 317 | APCI m/z 370.0 [M + H]+ | 318 | APCI m/z 331.1 [M + H]+ |
| 319 | APCI m/z 387.4 [M + H]+ | 320 | APCI m/z 316.1 [M + H]+ |
| 321 | APCI m/z 316.0 [M + H]+ | 322 | APCI m/z 488.0 [M + H]+ |
| 323 | APCI m/z 386.0 [M + H]+ | 324 | APCI m/z 470 [M + H]+ |
| 325 | APCI m/z 404.0 [M + H]+ | 326 | APCI m/z 363.0 [M + H]+ |
| 327 | APCI m/z 412 [M + H]+ | 328 | APCI m/z 330.1 [M + H]+ |
| 329 | APCI m/z 350.0 [M + H]+ | 330 | APCI m/z 415.0 [M + H]+ |
| 331 | APCI m/z 450.0 [M + H]+ | 332 | APCI m/z 423.4 [M + H]+ |
| 333 | APCI m/z 334.0 [M + H]+ | 334 | APCI m/z 454.3 [M + H]+ |
| 335 | APCI m/z 366.0 [M + H]+ | 336 | APCI m/z 457.0 [M + H]+ |
| 337 | APCI m/z 468.0 [M + H]+ | 338 | APCI m/z 365.0 [M + H]+ |
| 339 | APCI m/z 502.0 [M + H]+ | 340 | APCI m/z 388.4 [M + H]+ |
| 341 | APCI m/z 370.4 [M + H]+ | 342 | APCI m/z 465.0 [M + H]+ |
| 343 | APCI m/z 384.0 [M + H]+ | 344 | APCI m/z 346.0 [M + H]+ |
| 345 | APCI m/z 445 [M + H]+ | 346 | APCI m/z 352 [M + H]+ |
| 347 | APCI m/z 352 [M + H]+ | 348 | APCI m/z 427 [M + H]+ |
| 349 | APCI m/z 338 [M + H]+ | 350 | APCI m/z 366 [M + H]+ |
| 351 | APCI m/z 346.0 [M + H]+ | 352 | APCI m/z 430.0 [M + H]+ |
| 353 | APCI m/z 430.0 [M + H]+ | 354 | APCI m/z 333.4 [M + H]+ |
| 355 | APCI m/z 395 [M + H]+ | 356 | APCI m/z 311.0 [M + H]+ |
| 357 | APCI m/z 324.1 [M + H]+ | 358 | APCI m/z 338.1 [M + H]+ |

### BIOLOGICAL EXAMPLES

### Biological Example B-1

Compounds described herein were assayed for their ability to inhibit the hydrolysis of NAD+ by the protein CD38. The human and mouse recombinant enzyme assays measure the inhibition of the enzyme activity by compounds using recombinant enzymes and substrates in a buffered cell-free system. The assay conditions closely mimic cellular environments. Dose responses were measured using an assay to detect the hydrolysis of NAD+. All experiments were performed in the 384-well format. Generally, 0.1 μL of DMSO containing varying concentrations of the test compound was mixed with 10 μL of the enzyme reagent solution. Enzyme reactions were initiated with the addition of 10 μL of a solution containing NAD+ substrate. Subsequent detection of remaining NAD+ was determined by first converting NAD+ to NADH using alcohol dehydrogenase, then using the resulting NADH to reduce resazurine to the fluorescent product resorufin. The final assay conditions were as follows: 0.4 nM human CD38 and 62.5 μM NAD+ in 50 mM HEPES, pH 7.5, 1 mM CHAPS, 1 mM EDTA. Following an incubation of 60 min at ambient temperature, 10 μL of 120 mM ethanol+20 U/ml alcohol dehydrogenase+30 mM semicarbazide+0.03 mM CD38 inhibitor in 50 mM HEPES, pH 7.5, 0.2 mg/ml BSA, was added and incubated at ambient temperature for 15 min. Then 10 μl 0.32 mM NaOH was added to stop the ADH reaction (plates incubated at ambient temperature for 15 min), followed by 30 μL of 0.05 mM resazurine+1000 mU/ml Diaphorase in 200 mM Tris-HCl, pH 7.7 for 15 min at ambient temperature. The plates were read for fluorescence (Excitation/Emission=540 nm/590 nm) using an EnVision plate reader. The potency measurements for compounds, are quantified and represented as IC50 (the concentration of compounds that inhibits 50% activity). Results are shown in Table 4. Note that, in Table 4, the “Compound No” corresponds to the compound numbers in Table 1.

TABLE 4

| | mCD38 | hCD38 | | mCD38 | hCD38 |
|---|---|---|---|---|---|
| | BioChem | BioChem | | BioChem | BioChem |
| Compound | IC50 | IC50 | Compound | IC50 | IC50 |
| Number | (nM) | (nM) | Number | (nM) | (nM) |
| 1 | 358.0 | 243.0 | 2 | 1007.0 | 345.0 |
| 3 | 234.0 | 1090.0 | 4 | 491.0 | 1127.0 |
| 5 | 420.0 | 313.0 | 6 | 1583.0 | 352.0 |
| 7 | 223.0 | 549.0 | 8 | 8.0 | 10.0 |
| 9 | 432.0 | 1237.0 | 10 | 433.0 | 1193.0 |
| 11 | 741.0 | 2112.0 | 12 | 122.0 | 296.0 |
| 13 | 8.0 | 6.0 | 14 | 17.0 | 91.0 |
| 15 | 748.0 | 1142.0 | 16 | 760.0 | 1513.0 |
| 17 | 27.0 | 67.0 | 18 | 6.0 | 6.0 |
| 19 | 9.0 | 7.0 | 20 | 8.0 | 9.0 |
| 21 | 3.0 | 3.0 | 22 | 70.0 | 496.0 |
| 23 | 110.0 | 614.0 | 24 | 45.0 | 104.0 |
| 25 | 13.0 | 13.0 | 26 | 7.0 | 16.0 |
| 27 | 64.0 | 448.0 | 28 | 10.0 | 46.0 |
| 29 | 110.0 | 389.0 | 30 | 14.0 | 85.0 |
| 31 | 205.0 | 479.0 | 32 | 1.0 | 3.0 |
| 33 | 228.0 | 313.0 | 34 | 6.0 | 4.0 |
| 35 | 68.0 | 210.0 | 36 | 14.0 | 5.0 |
| 37 | 6.0 | 4.0 | 38 | 7.0 | 10.0 |
| 39 | 4.0 | 6.0 | 40 | 2.0 | 6.0 |
| 41 | 244.0 | 1211.0 | 42 | 8.0 | 9.0 |
| 43 | 32.0 | 200.0 | 44 | 66.0 | 98.0 |
| 45 | 44.0 | 82.0 | 46 | 248.0 | 562.0 |
| 47 | 100.0 | 417.0 | 48 | 46.0 | 244.0 |
| 49 | 29.0 | 88.0 | 50 | 855.0 | 1441.0 |
| 51 | 487.0 | 1199.0 | 52 | 292.0 | 678.0 |
| 53 | 10.0 | 10.0 | 54 | 38.0 | 189.0 |
| 55 | 10.0 | 5.0 | 56 | 6.0 | 5.0 |
| 57 | 36.0 | 187.0 | 58 | 1037.0 | 2182.0 |
| 59 | 18.0 | 25.0 | 60 | 12.0 | 61.0 |
| 61 | 13.0 | 73.0 | 62 | 12.0 | 61.0 |
| 63 | 163.0 | 404.0 | 64 | 7.0 | 34.0 |
| 65 | 62.0 | 224.0 | 66 | 190.0 | 91.0 |
| 67 | 9.0 | 4.0 | 68 | 6.0 | 16.0 |
| 69 | 2.0 | 3.0 | 70 | 1.0 | 5.0 |
| 71 | 25.0 | 15.0 | 72 | 19.0 | 4.0 |
| 73 | 33.0 | 60.0 | 74 | 10.0 | 3.0 |
| 75 | 19.0 | 41.0 | 76 | 3198.0 | 905.0 |
| 77 | 32.0 | 131.0 | 78 | 16.0 | 79.0 |
| 79 | 12.0 | 21.0 | 80 | 9.0 | 20.0 |
| 81 | 4.0 | 13.0 | 82 | 3.0 | 10.0 |
| 83 | 6.0 | 11.0 | 84 | 2.0 | 12.0 |
| 85 | 2.0 | 7.0 | 86 | 219.0 | 194.0 |
| 87 | 138.0 | 562.0 | 88 | 15.0 | 35.0 |
| 89 | 10.0 | 22.0 | 90 | 13.0 | 4.0 |
| 91 | 19.0 | 38.0 | 92 | 13.0 | 22.0 |
| 93 | 5.0 | 2.0 | 94 | 2.0 | 2.0 |
| 95 | 6.0 | 4.0 | 96 | 3.0 | 23.0 |
| 97 | 3.0 | 24.0 | 98 | 10.0 | 106.0 |
| 99 | 7.0 | 80.0 | 100 | 61.0 | 263.0 |
| 101 | 42.0 | 210.0 | 102 | 7.0 | 23.0 |
| 103 | 30.0 | 146.0 | 104 | 10.0 | 24.0 |
| 105 | 24.0 | 80.0 | 106 | 9.0 | 22.0 |
| 107 | 2.0 | 9.0 | 108 | 33.0 | 711.0 |
| 109 | 66.0 | 312.0 | 110 | 20.0 | 108.0 |
| 111 | 38.0 | 129.0 | 112 | 158.0 | 639.0 |
| 113 | 166.0 | 569.0 | 114 | 150.0 | 173.0 |
| 115 | 494.0 | 1257.0 | 116 | 185.0 | 507.0 |
| 117 | 1.0 | 4.0 | 118 | 3.0 | 17.0 |
| 119 | 1.0 | 10.0 | 120 | 2.0 | 10.0 |
| 121 | 1.0 | 14.0 | 122 | 1.0 | 10.0 |
| 123 | 1.0 | 10.0 | 124 | 1.0 | 17.0 |
| 125 | 4.0 | 19.0 | 126 | 84.0 | 841.0 |
| 127 | 2.0 | 2.0 | 128 | 1.0 | 9.0 |
| 129 | 1.0 | 2.0 | 130 | 44.0 | 488.0 |
| 131 | 23.0 | 66.0 | 132 | 55.0 | 115.0 |
| 133 | 135.0 | 1734.0 | 134 | 5.0 | 37.0 |
| 135 | 15.0 | 76.0 | 136 | 6.0 | 23.0 |
| 137 | 7.0 | 8.0 | 138 | 25.0 | 73.0 |
| 139 | 232.0 | 1013.0 | 140 | 117.0 | 747.0 |
| 141 | 28.0 | 761.0 | 142 | 161.0 | 1715.0 |
| 143 | 1972.0 | 2039.0 | 144 | 14.0 | 3.0 |
| 145 | 57.0 | 136.0 | 146 | 23.0 | 62.0 |
| 147 | 6.0 | 2.0 | 148 | 23.0 | 70.0 |
| 149 | 13.0 | 43.0 | 150 | 4986.0 | 1061.0 |
| 151 | 1.0 | 14.0 | 152 | 1.0 | 13.0 |
| 153 | 2.0 | 25.0 | 154 | 1.0 | 11.0 |
| 155 | 1.0 | 24.0 | 156 | 4.0 | 30.0 |
| 157 | 48.0 | 121.0 | 158 | 185.0 | 228.0 |
| 159 | 5.0 | 9.0 | 160 | 4.0 | 27.0 |
| 161 | 4.0 | 26.0 | 162 | 1.0 | 3.0 |
| 163 | 82.0 | 375.0 | 164 | 11.0 | 44.0 |
| 165 | 1.0 | 19.0 | 166 | 2.0 | 21.0 |
| 167 | 46.0 | 214.0 | 168 | 47.0 | 313.0 |
| 169 | 15.0 | 22.0 | 170 | 32.0 | 47.0 |
| 171 | 3.0 | 15.0 | 172 | 5.0 | 21.0 |
| 173 | 2.0 | 8.0 | 174 | 4.0 | 8.0 |
| 175 | 335.0 | 330.0 | 176 | 19.0 | 28.0 |
| 177 | 9.0 | 35.0 | 178 | 13.0 | 57.0 |
| 179 | 142.0 | 1375.0 | 180 | 8.0 | 78.0 |
| 181 | 2.0 | 14.0 | 182 | 2.0 | 11.0 |
| 183 | 2.0 | 7.0 | 184 | 16.0 | 56.0 |
| 185 | 14.0 | 30.0 | 186 | 5.0 | 10.0 |
| 187 | 1.9 | 3.1 | 188 | 661.2 | 1969.1 |
| 189 | 260.8 | 332.7 | 190 | 427.1 | 502.0 |
| 191 | 4.5 | 35.3 | 192 | 5.5 | 46.1 |
| 193 | 39.8 | 131.2 | 194 | 63.6 | 111.0 |
| 195 | 9.9 | 14.6 | 196 | 19.3 | 25.4 |
| 197 | 1.0 | 4.5 | 198 | 1.0 | 3.8 |
| 199 | 3.1 | 15.2 | 200 | 1.9 | 9.3 |
| 201 | 1.3 | 2.6 | 202 | 1.1 | 2.8 |
| 203 | 12.1 | 161.4 | 204 | 8.8 | 122.9 |
| 205 | 1.6 | 4.0 | 206 | 1.7 | 3.9 |
| 207 | 7.2 | 24.8 | 208 | 531.3 | 961.5 |
| 209 | 35.0 | 104.0 | 210 | 1.2 | 1.6 |
| 211 | 29.8 | 46.2 | 212 | 104.9 | 832.7 |
| 213 | 9.1 | 15.2 | 214 | 52.9 | 322.9 |
| 215 | 1.6 | 2.0 | 216 | 49.3 | 66.6 |
| 217 | 19.7 | 36.4 | 218 | 11.6 | 25.2 |
| 219 | 99.0 | 361.2 | 220 | 52.2 | 145.6 |
| 221 | 2.2 | 3.6 | 222 | 203.5 | 932.8 |
| 223 | 458.5 | 1634.2 | 224 | 1.1 | 5.6 |
| 225 | 3.8 | 7.0 | 226 | 12.7 | 20.4 |
| 227 | 2.4 | 9.3 | 228 | 4.7 | 16.2 |
| 229 | 2.5 | 11.2 | 230 | 1.7 | 4.3 |
| 231 | 9.0 | 72.2 | 232 | 8.1 | 53.7 |
| 233 | 1.1 | 6.0 | 234 | 1.4 | 2.4 |
| 235 | 1.6 | 3.8 | 236 | 27.2 | 46.4 |
| 237 | 29.5 | 33.8 | 238 | 40.9 | 43.2 |
| 239 | 33.6 | 61.2 | 240 | 13.3 | 27.9 |
| 241 | 14.4 | 16.7 | 242 | 21.3 | 28.8 |
| 243 | 14.6 | 28.1 | 244 | 1.0 | 1.9 |
| 245 | 1.0 | 2.8 | 246 | 61.8 | 82.9 |
| 247 | 139.1 | 233.1 | 248 | 13.8 | 10.7 |
| 249 | 45.2 | 11.9 | 250 | 32.0 | 14.1 |
| 251 | 58.7 | 207.3 | 252 | 183.7 | 255.4 |
| 253 | 124.5 | 1695.2 | 254 | 31.2 | 93.5 |
| 255 | 28.7 | 71.5 | 256 | 22.5 | 68.4 |
| 257 | 43.4 | 124.1 | 258 | 36.4 | 71.6 |
| 259 | 15.6 | 46.1 | 260 | 161.4 | 271.1 |
| 261 | 39.1 | 86.3 | 262 | 232.4 | 1840.2 |
| 263 | 269.6 | 252.5 | 264 | 15.3 | 40.9 |
| 265 | 35.9 | 83.4 | 266 | 327.4 | 707.7 |
| 267 | 0.3 | 2.5 | 268 | 96.3 | 35.4 |
| 269 | 175.0 | 198.9 | 270 | 43.9 | 13.7 |
| 271 | 415.2 | 2151.2 | 272 | 224.1 | 1340.8 |
| 273 | 53.7 | 71.9 | 274 | 185.1 | 404.5 |
| 275 | 15.4 | 31.5 | 276 | 4.4 | 1.4 |
| 277 | 10.7 | 7.9 | 278 | 67.9 | 489.6 |
| 279 | 42.6 | 5.1 | 280 | 3.2 | 10.7 |
| 281 | 1.3 | 4.1 | 282 | 0.7 | 2.4 |
| 283 | 4.3 | 18.4 | 284 | 59.7 | 277.0 |
| 285 | 1.0 | 8.6 | 286 | 1.2 | 8.1 |
| 287 | 0.7 | 3.1 | 288 | 1.0 | 10.3 |
| 289 | 1.0 | 7.0 | 290 | 191.4 | 1218.0 |
| 291 | 3.4 | 4.1 | 292 | 7.1 | 21.4 |
| 293 | 36.2 | 58.5 | 294 | 175.5 | 251.4 |
| 295 | 3.6 | 18.5 | 296 | 9.9 | 8.7 |
| 297 | 90.8 | 413.8 | 298 | 165.7 | 319.3 |
| 299 | 74.8 | 287.7 | 300 | 1.6 | 15.7 |
| 301 | 198.7 | 621.0 | 302 | 117.8 | 368.9 |
| 303 | 62.4 | 295.9 | 304 | 244.1 | 1271.1 |
| 305 | 62.4 | 485.2 | 306 | 81.7 | 357.1 |
| 307 | 1.0 | 1.5 | 308 | 94.3 | 125.2 |
| 309 | 1.3 | 4.2 | 310 | 105.0 | 354.1 |
| 311 | 3.8 | 13.5 | 312 | 10.2 | 26.5 |
| 313 | 198.0 | 760.7 | 314 | 7.5 | 8.9 |
| 315 | 1.8 | 13.7 | 316 | 2.0 | 15.0 |
| 317 | 41.7 | 79.1 | 318 | 13.4 | 18.5 |
| 319 | 6.0 | 3.6 | 320 | 217.6 | 949.5 |
| 321 | 32.2 | 133.2 | 322 | 6.9 | 41.8 |
| 323 | 10.6 | 22.1 | 324 | 2.1 | 15.9 |
| 325 | 19.3 | 29.8 | 326 | 1.4 | 3.4 |
| 327 | 2.6 | 22.1 | 328 | 11.5 | 41.4 |
| 329 | 10.7 | 34.0 | 330 | 9.8 | 5.0 |
| 331 | 4.7 | 26.0 | 332 | 1.5 | 11.0 |
| 333 | 3.4 | 12.8 | 334 | 5.4 | 12.4 |
| 335 | 13.5 | 28.7 | 336 | 1.3 | 12.3 |
| 337 | 1.9 | 10.4 | 338 | 1.5 | 1.8 |
| 339 | 2.0 | 17.3 | 340 | 6.3 | 11.9 |
| 341 | 3.8 | 8.7 | 342 | 2.9 | 19.9 |
| 343 | 13.6 | 24.3 | 344 | 46.9 | 417.2 |
| 345 | 1.0 | 3.3 | 346 | 30.1 | 108.8 |
| 347 | 6.2 | 81.9 | 348 | 1.0 | 2.5 |
| 349 | 28.2 | 128.0 | 350 | 11.3 | 107.3 |
| 351 | 267.4 | 675.8 | 352 | 20.6 | 25.4 |
| 353 | 4.2 | 20.8 | 354 | 16.9 | 4.2 |
| 355 | 1.1 | 3.5 | 356 | 10.0 | 4.3 |
| 357 | 10.2 | 5.7 | 358 | 5.8 | 4.3 |

N.D. = Not Determined

### Biological Example B-2

The compounds described herein were also assayed for their ability to inhibit the endogenous CD38 in a native cellular environment in the cellular CD38 assay, which measures the ability of the compound to modulate cellular NAD levels. Leukemic HL60 cells were grown in RPMI Medium, along with 10% fetal bovine serum, in a humidified incubator with an atmosphere of 95% air and 5% CO₂ at 37° C. The assays were initiated by plating 20 μL of HL60 cells in culture medium, at a density of 20000 cells per well to a 384-well Corning™ Multiwell Plates. Compounds in DMSO were added to the plates in a volume of 120 nL using the Labcyte Echo Liquid Handlers. 5 μL of a 120 nM all-trans retinoic acid solution in assay medium is added to each well. The plates are then incubated for 24 hours. 50 μL of a readout-solution containing 0.2 U/mL Diaphorase enzyme, 40 uM resazurin, 10 uM FMN, 0.8 U/mL Alcohol dehydrogenase, 3% ethanol, 0.4 mg/mL bovine serum albumin, 0.2% Triton X-100 in 100 mM Tris-HCl, 30 mM EDTA, pH 8.4. The plates were read for fluorescence (Excitation/Emission=540 nm/590 nm) using an EnVision plate reader after 60 mins of incubation at ambient temperature. Results are shown in Table 5. Note that, in Table 5, the “Compound No” corresponds to the compound numbers in Table 1.

TABLE 5

| | hCD38 HL60 ATRA | | hCD38 HL60 ATRA |
|---|---|---|---|
| | NAD Cell | | NAD Cell |
| Compound | Diaphorase IC50 | Compound | Diaphorase IC50 |
| Number | (nM) | Number | (nM) |
| 1 | 2003 | 2 | 3060 |
| 3 | 6780 | 4 | 8508 |
| 5 | 7816 | 6 | 8686 |
| 7 | 4463 | 8 | 280 |
| 9 | 5975 | 10 | 4345 |
| 11 | 8045 | 12 | 3988 |
| 13 | 568 | 14 | 3292 |
| 15 | 9963 | 16 | 7387 |
| 17 | 1402 | 18 | 415 |
| 19 | 345 | 20 | 365 |
| 21 | 273 | 22 | 802 |
| 23 | 3885 | 24 | 1664 |
| 25 | 468 | 26 | 629 |
| 27 | 10395 | 28 | 2264 |
| 29 | 8549 | 30 | 2831 |
| 31 | 15743 | 32 | 264 |
| 33 | 13729 | 34 | 509 |
| 35 | 3574 | 36 | 187 |
| 37 | 339 | 38 | 873 |
| 39 | 298 | 40 | 959 |
| 42 | 903 | 43 | 7493 |
| 44 | 5817 | 45 | 4690 |
| 46 | 10185 | 47 | 9150 |
| 48 | 3763 | 49 | 3371 |
| 52 | 11364 | 53 | 675 |
| 54 | 8279 | 55 | 522 |
| 56 | 263 | 57 | 4548 |
| 59 | 769 | 60 | 4806 |
| 61 | 7848 | 62 | 6709 |
| 63 | 5911 | 64 | 2425 |
| 65 | 7692 | 66 | 3434 |
| 67 | 409 | 68 | 1324 |
| 69 | 481 | 70 | 770 |
| 71 | 1435 | 72 | 295 |
| 73 | 1994 | 74 | 349 |
| 75 | 1358 | 77 | 3356 |
| 78 | 1115 | 79 | 2326 |
| 80 | 1524 | 81 | 978 |
| 82 | 1082 | 83 | 2771 |
| 84 | 1114 | 85 | 765 |
| 86 | 8805 | 87 | 10117 |
| 88 | 1517 | 89 | 915 |
| 90 | 369 | 91 | 2082 |
| 92 | 1177 | 93 | 187 |
| 94 | 241 | 95 | 280 |
| 96 | 1634 | 97 | 1528 |
| 98 | 4741 | 99 | 5130 |
| 100 | 6197 | 101 | 8306 |
| 102 | 1007 | 103 | 1623 |
| 104 | 1362 | 105 | 1671 |
| 106 | 1799 | 107 | 1291 |
| 109 | 20000 | 110 | 2553 |
| 111 | 5492 | 114 | 4333 |
| 116 | 8162 | 117 | 350 |
| 118 | 1623 | 119 | 1579 |
| 120 | 1127 | 121 | 1045 |
| 122 | 1012 | 123 | 952 |
| 124 | 1396 | 125 | 2479 |
| 126 | 20000 | 127 | 335 |
| 128 | 928 | 129 | 267 |
| 130 | 10266 | 131 | 2913 |
| 132 | 5560 | 134 | 3017 |
| 135 | 1322 | 136 | 1537 |
| 137 | 867 | 138 | 3006 |
| 144 | 129 | 145 | 2545 |
| 146 | 1700 | 147 | 151 |
| 148 | 1894 | 149 | 1382 |
| 151 | 1970 | 152 | 2353 |
| 153 | 3130 | 154 | 3147 |
| 155 | 2299 | 156 | 2299 |
| 157 | 7235 | 158 | 3977 |
| 159 | 1088 | 160 | 20000 |
| 161 | 2450 | 162 | 718 |
| 163 | 11741 | 164 | 3997 |
| 165 | 2980 | 166 | 2235 |
| 167 | 20000 | 168 | 8650 |
| 169 | 2252 | 170 | 4864 |
| 171 | 3732 | 172 | 2951 |
| 173 | 1248 | 174 | 1776 |
| 175 | 6460 | 187 | 253 |
| 189 | 8514 | 190 | 20000 |
| 191 | 3281 | 192 | 3761 |
| 193 | 11110 | 194 | 7157 |
| 195 | 1577 | 196 | 3194 |
| 197 | 709 | 198 | 1130 |
| 199 | 2626 | 200 | 2307 |
| 201 | 317 | 202 | 229 |
| 203 | 7874 | 204 | 8565 |
| 205 | 410 | 206 | 576 |
| 207 | 3694 | 209 | 8537 |
| 210 | 303 | 211 | 2007 |
| 213 | 1475 | 214 | 9856 |
| 215 | 214 | 216 | 9424 |
| 217 | 1966 | 218 | 2222 |
| 219 | 10772 | 220 | 4953 |
| 221 | 1127 | 224 | 707 |
| 225 | 1077 | 226 | 5206 |
| 227 | 2070 | 228 | 3876 |
| 229 | 4002 | 230 | 1463 |
| 231 | 10213 | 232 | 7295 |
| 233 | 1042 | 234 | 557 |
| 235 | 376 | 236 | 1866 |
| 237 | 2202 | 238 | 2384 |
| 239 | 3134 | 240 | 2060 |
| 241 | 2050 | 242 | 2369 |
| 243 | 3720 | 244 | 499 |
| 245 | 749 | 246 | 8772 |
| 247 | 14401 | 248 | 874 |
| 249 | 838 | 250 | 924 |
| 251 | 5106 | 252 | 4875 |
| 254 | 3671 | 255 | 2756 |
| 256 | 3607 | 257 | 4525 |
| 258 | 5581 | 259 | 2279 |
| 260 | 10613 | 261 | 3098 |
| 263 | 12547 | 264 | 2103 |
| 265 | 4175 | 267 | 403 |
| 268 | 1396 | 269 | 3948 |
| 270 | 1126 | 273 | 2471 |
| 274 | 10768 | 275 | 2209 |
| 276 | 209 | 277 | 2072 |
| 279 | 865 | 280 | 1503 |
| 281 | 1328 | 282 | 655 |
| 283 | 5694 | 284 | 11003 |
| 285 | 1244 | 286 | 3733 |
| 287 | 882 | 288 | 2580 |
| 289 | 1898 | 291 | 2619 |
| 292 | 6613 | 293 | 2833 |
| 294 | 4478 | 295 | 2313 |
| 296 | 1138 | 299 | 4100 |
| 300 | 2121 | 303 | 10363 |
| 307 | 254 | 308 | 6273 |
| 309 | 933 | 311 | 2146 |
| 312 | 2790 | 314 | 1371 |
| 315 | 2283 | 316 | 1471 |
| 318 | 1921 | 319 | 964 |
| 357 | 940 | 358 | 1010 |

N.D. = Not Determined

### Biological Example B-3

### Tissue Levels of Nicotinamide in Mouse Tissues Following Oral Administration of Compound 148 to Aged C57BL/6 Mice

0.1% Tween 80/0.5% HPMC or compound 148 prepared with 0.1% Tween 80/0.5% HPMC was orally administered at 100 mg/kg BID to 72-week-old male C57BL/6J mice. 4 h after the 3^{rd} administration, each mouse was euthanized, and tissues collected.

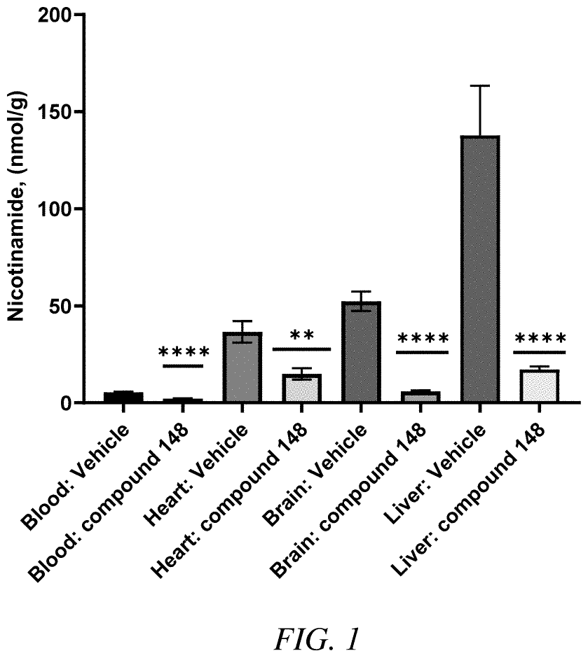

Whole blood was collected and placed into pre-chilled K₂EDTA microtainer tube, rotated 3-4 times to ensure anticoagulant mixing. An aliquot from the whole blood collection was added to 10 volumes of 0.5M PCA (perchloric acid), inverted 3-4 times to mix well, and then frozen on dry ice. While under isoflurane after cardiac puncture for blood collection, the following tissues were harvested in this order: heart, liver lobe, then by brain. All tissues were processed using pre-chilled freeze clamp. The frozen tissues were placed in pre-frozen labeled 2 mL Eppendorf tubes. The tissues were stored at −80° C. until processing. Upon processing, each tissue was cryomilled in the frozen state to form a powder. Frozen powdered tissue was weighed into pre-frozen tubes. Approximately 10 volumes of 0.5M PCA per weight of tissue were added to the tube followed by freezing until analysis. Upon analysis, blood and tissue samples were thawed on ice, followed by homogenization via TissueLyzer. Samples were centrifuged, the supernatant was collected and filtered, and the nicotinamide concentration in the supernatant of sample was measured using LC/MS. The nicotinamide concentration in each tissue is shown in FIG. 1.

The results of FIG. 1 indicated that the treatment with compound 148 decreased nicotinamide levels in the mouse blood, heart, brain and liver tissues.

### Biological Example B-4

### In Vitro Determination of Direct and Time-Dependent Inhibition of CYP450 Enzymes

Direct inhibition: The potential of direct inhibition of CYP1A2, 2B6, 2C9, 2C19, 2D6 and 3A4 by test compounds was assessed in human liver microsomes (HLM) in vitro using standard methods (Grimm et al, “The Conduct of in Vitro Studies to Address Time-Dependent Inhibition of Drug-Metabolizing Enzymes: A Perspective of the Pharmaceutical Research and Manufacturers of America”, Drug Metabolism and Disposition, 37 (7): 1355, 2009). For 3A4, the % activity was measured using both midazolam and testosterone as probes. Each compound at 3, 10, and 50 μM was incubated with HLM, NADPH and CYP isoenzyme specific probe substrates at 37° C. Inhibition of the probe substrate metabolism was quantified using LC/MS/MS. The inhibition of each P450 enzyme was measured as the percentage decrease in the activity of marker metabolite formation compared to non-inhibited controls (=100% activity). Known chemical inhibitors specific for the individual CYP isoenzymes were evaluated in parallel as positive controls and these compounds produced CYP inhibition consistent with published results (Walsky & Obach RS. “Validated assays for human cytochrome P450 activities”. Drug Metab Dispos. 32(6):647-60, 2004). Results are shown in Tables 6 and 7. Note that, in Tables 6 and 7, the “Compound No” corresponds to the compound numbers in Table 1.

Comparator Compound A is described in WO2021/207186 and has the following structure:

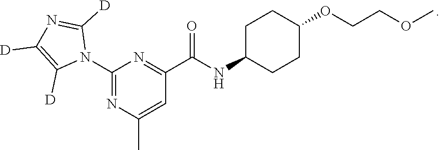 [SMILES: [2H]c1nc([2H])n(-c2nc(C)cc(C(=O)N[C@H]3CC[C@H](OCCOC)CC3)n2)c1[2H]]

TABLE 6

| Compound | Conc | CYP1A2 | CYP2B6 | CYP2C9 | CYP2C19 |
|---|---|---|---|---|---|
| Number | (uM) | T0 | T0 | T0 | T0 |
| Comparator | 3 | 98 | 103 | 88 | 77 |
| Compound | 10 | 98 | 98 | 64 | 49 |
| A | 50 | 92 | 57 | 24 | 17 |
| | estimated | >50 | >50 | <50 | ~10 |
| | IC₅₀ | | | | |
| 145 | 3 | 94 | 101 | 97 | 92 |
| | 10 | 92 | 98 | 99 | 93 |
| | 50 | 79 | 99 | 96 | 89 |
| | estimated | >50 | >50 | >50 | >50 |
| | IC₅₀ | | | | |
| 199 | 3 | 96 | 95 | 101 | 92 |
| | 10 | 96 | 80 | 96 | 85 |
| | 50 | 85 | 53 | 69 | 39 |
| | estimated | >50 | >50 | >50 | <50 |
| | IC₅₀ | | | | |
| 249 | 3 | 99 | 98 | 106 | 95 |
| | 10 | 93 | 98 | 103 | 96 |
| | 50 | 81 | 92 | 92 | 74 |
| | estimated | >50 | >50 | >50 | >50 |
| | IC₅₀ | | | | |
| 250 | 3 | 88 | 101 | 99 | 88 |
| | 10 | 69 | 99 | 94 | 88 |
| | 50 | 34 | 89 | 89 | 66 |
| | estimated | <50 | >50 | >50 | >50 |
| | IC₅₀ | | | | |
| 256 | 3 | 94 | 103 | 90 | 97 |
| | 10 | 92 | 101 | 94 | 96 |
| | 50 | 78 | 91 | 86 | 88 |
| | estimated | >50 | >50 | >50 | >50 |
| | IC₅₀ | | | | |
| 289 | 3 | 100 | 98 | 96 | 83 |
| | 10 | 95 | 95 | 81 | 63 |
| | 50 | 81 | 79 | 49 | 23 |
| | estimated | >50 | >50 | <50 | <50 |
| | IC₅₀ | | | | |
| 295 | 3 | 101 | 96 | 91 | 108 |
| | 10 | 98 | 91 | 85 | 100 |
| | 50 | 97 | 79 | 63 | 79 |
| | estimated | >50 | >50 | >50 | >50 |
| | IC₅₀ | | | | |

N.D. = Not Determined

TABLE 7

| Compound | Conc | CYP2D6 | CYP3A4-T | CYP3A4-M |
|---|---|---|---|---|
| Number | (uM) | T0 | T0 | T0 |
| Comparator | 3 | 95 | 100 | 98 |
| Compound A | 10 | 82 | 97 | 99 |
| | 50 | 42 | 103 | 91 |
| | estimated | <50 | >50 | >50 |
| | IC₅₀ | | | |
| 145 | 3 | 93 | 96 | 99 |
| | 10 | 73 | 98 | 89 |
| | 50 | 47 | 95 | 75 |
| | estimated | <50 | >50 | >50 |
| | IC₅₀ | | | |
| 199 | 3 | N.D. | 98 | 93 |
| | 10 | N.D. | 96 | 92 |
| | 50 | N.D. | 79 | 79 |
| | estimated | N.D. | >50 | >50 |
| | IC₅₀ | | | |
| 249 | 3 | 104 | 99 | 108 |
| | 10 | 103 | 100 | 103 |
| | 50 | 87 | 92 | 100 |
| | estimated | >50 | >50 | >50 |
| | IC₅₀ | | | |
| 250 | 3 | 103 | 104 | 96 |
| | 10 | 86 | 106 | 89 |
| | 50 | 47 | 85 | 73 |
| | estimated | <50 | >50 | >50 |
| | IC₅₀ | | | |
| 256 | 3 | 89 | 96 | 88 |
| | 10 | 87 | 99 | 97 |
| | 50 | 56 | 95 | 82 |
| | estimated | >50 | >50 | >50 |
| | IC₅₀ | | | |
| 289 | 3 | 98 | 100 | 147 |
| | 10 | 98 | 97 | 148 |
| | 50 | 64 | 72 | 96 |
| | estimated | >50 | >50 | >50 |
| | IC₅₀ | | | |
| 295 | 3 | 95 | 103 | 92 |
| | 10 | 79 | 106 | 93 |
| | 50 | 36 | 86 | 101 |
| | estimated | <50 | >50 | >50 |
| | IC₅₀ | | | |

N.D. = Not Determined

Time dependent inhibition: An assessment of the time-dependent Inhibitory potential of test compounds against the principal human cytochrome P450 isozymes was carried out using standard methods (Grimm et al, “The Conduct of in Vitro Studies to Address Time-Dependent Inhibition of Drug-Metabolizing Enzymes: A Perspective of the Pharmaceutical Research and Manufacturers of America”, Drug Metabolism and Disposition, 37 (7): 1355, 2009). Pooled human microsomes and selective CYP probe substrates were used for in vitro assessment of test compound from 0.1 to 30 μM as time-dependent inhibitor of seven human hepatic cytochrome P450 isozymes (CYP1A2, 2B6, 2C8, 2C9, 2C19, 2D6, and 3A4). Each compound was pre-incubated at 37° C. plus and minus NADPH for 30 min, and with 0 min pre-incubation for assessment of potential time dependent inhibition. LC-MS/MS was used to quantify metabolite formation. IC₅₀ was calculated at each condition and the occurrence of any time dependent inhibition was then expressed as the IC₅₀ fold shift between 30 min pre-incubation plus NADPH and 0 min preincubation. Compound 148 showed reversible inhibition for 1A2, 2B6, and 2D6 with IC₅₀ at 10 μM, 26.2 μM, and 13.7 μM at 0 min pre-incubation respectively. Compound 148 did not show any indication of time dependent inhibition since the IC₅₀ shift was not greater than 1.5-fold in any isozymes. Results are shown in Tables 8 and 9. Note that, in Tables 8 and 9, the “Compound No” corresponds to the compound numbers in Table 1.

TABLE 8

| Compund | Pre- | | | | |
|---|---|---|---|---|---|
| Number | incubation | CYP1A2 | CYP2B6 | CYP2C8 | CYP2C9 |
| 148 | 0 min | 10.0 | 26.2 | >30 | >30 |
| | 30 min (NO | 12.0 | 24.4 | >30 | >30 |
| | NADPH) | | | | |
| | 30 min | 11.8 | >30 | >30 | >30 |
| | (NADPH) | | | | |
| | shift | 0.8 | none | none | none |

TABLE 9

| Compund | Pre- | | | CYP3A4- | CYP3A4- |
|---|---|---|---|---|---|
| Number | incubation | CYP2C19 | CYP2D6 | M | T |
| 148 | 0 min | >30 | 13.7 | >30 | >30 |
| | 30 min (NO | >30 | 9.1 | >30 | >30 |
| | NADPH) | | | | |
| | 30 min | >30 | 12.3 | >30 | >30 |
| | (NADPH) | | | | |
| | shift | none | 1.1 | none | none |

1. A compound of formula (I): 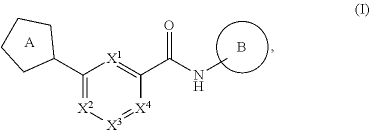
or a stereoisomer or tautomer thereof, or a pharmaceutically acceptable salt of any of the foregoing, wherein:
X¹ is N or CH;
X² is N or C(R^{x}), wherein R^{x} is H, halo, or C₁₋₆alkyl;
X³ is N or C(R^{y}), wherein R^{y} is H, —OH, C₁₋₆alkoxy, C₃₋₁₀cycloalkyl, 3-10 membered heterocyclyl, or C₁₋₆ alkyl,
wherein the C₁₋₆ alkoxy of R^{y} is optionally substituted with one or more C₁₋₆ alkoxy, the C₃₋₁₀cycloalkyl of R^{y} is optionally substituted with one or more halo, C₁₋₆alkoxy, or —OH, the 3-10 membered heterocyclyl of R^{y} is optionally substituted with one or more C₁₋₆alkyl, and the C₁₋₆ alkyl of R^{y} is optionally substituted with one or more halo or —OH;
X⁴ is N or C(R^{z}), wherein R^{z} is H, halo, —NH₂, C₁₋₆alkoxy, or C₁₋₆alkyl;
provided that at most two of X¹, X², X³, and X⁴ are N; 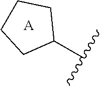
is:
(i) 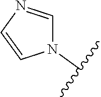 [SMILES: *n1ccnc1]
that is optionally substituted with one or more —C(O)—NH₂, or
(ii) 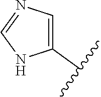 [SMILES: *c1cnc[nH]1]
that is optionally substituted with one or more C₁₋₆ alkyl, or
(iii) 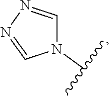 [SMILES: *n1cnnc1]
or
(iv) 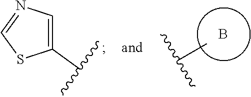
is:
(i) C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆ haloalkyl), phenyl, phenoxy, or pyridinyl,
wherein the C₁₋₆ alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
(ii) 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆ alkoxy, or phenyl, wherein the phenyl of R^{b} is optionally substituted with one or more C₁₋₆ haloalkyl, or
(iii) phenyl, wherein the phenyl is optionally substituted with one or more halo, or with C₁₋₆alkyl optionally substituted with —OH, or
(iv) pyridinyl, wherein the pyridinyl is optionally substituted with one or more halo, C₁-6haloalkyl, C₁₋₆ alkoxy optionally substituted with one or more halo, C₁₋₆ alkyl optionally substituted with —OH, or —O—C₃₋₁₀cycloalkyl optionally substituted with one or more halo;
provided that the compound of formula (I) or the stereoisomer or tautomer thereof, or the pharmaceutically acceptable salt of any of the foregoing, is not a compound selected from the compounds of Table 1X, Table 2X, Table 3X, Table 4X, Table 5X, or Table 6X, or a stereoisomer or tautomer thereof, or a pharmaceutically acceptable salt of any of the foregoing.

2. The compound of claim 1, or a stereoisomer or tautomer thereof, or a pharmaceutically acceptable salt of any of the foregoing, wherein at most two of X¹, X², X³, and X⁴ are N and at most three of X¹, X², X³, and X⁴ are not N.

3. The compound of claim 1, or a stereoisomer or tautomer thereof, or a pharmaceutically acceptable salt of any of the foregoing, wherein the compound is selected from the group consisting of compounds 2-7, 9, 11, 12, 14-19, 22-24, 27, 29, 31, 33-36, 38-49, 51-55, 57, 58, 60-70, 72-79, 81-155, and 158-358, or a stereoisomer or tautomer thereof, or a pharmaceutically acceptable salt of any of the foregoing.

4. A compound of formula (I-A2): 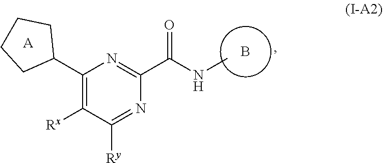
or a stereoisomer or tautomer thereof, or a pharmaceutically acceptable salt of any of the foregoing, wherein:
R^{x} is H, halo, or C₁₋₆alkyl;
R^{y} is H, —OH, C₁₋₆alkoxy, C₃₋₁₀cycloalkyl, 3-10 membered heterocyclyl, or C₁₋₆alkyl,
wherein the C₁₋₆ alkoxy of R^{y} is optionally substituted with one or more C₁₋₆alkoxy, the C₃₋₁₀cycloalkyl of R^{y} is optionally substituted with one or more halo, C₁₋₆alkoxy, or —OH, the 3-10 membered heterocyclyl of R^{y} is optionally substituted with one or more C₁₋₆alkyl, and the C₁₋₆ alkyl of R^{y} is optionally substituted with one or more halo or —OH; 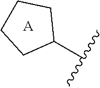
is:
(i) 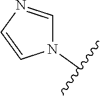 [SMILES: *n1ccnc1]
that is optionally substituted with one or more —C(O)—NH₂, or
(ii) 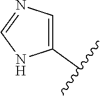 [SMILES: *c1cnc[nH]1]
that is optionally substituted with one or more C₁₋₆ alkyl, or
(iii) 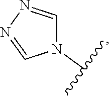 [SMILES: *n1cnnc1]
or
(iv) 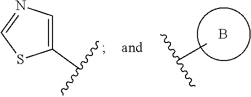
is:
(i) C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆ haloalkyl), phenyl, phenoxy, or pyridinyl,
wherein the C₁₋₆ alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
(ii) 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆ alkoxy, or phenyl, wherein the phenyl of R^{b} is optionally substituted with one or more C₁₋₆ haloalkyl, or
(iii) phenyl, wherein the phenyl is optionally substituted with one or more halo, or with C₁₋₆alkyl optionally substituted with —OH, or
(iv) pyridinyl, wherein the pyridinyl is optionally substituted with one or more halo, C₁₋₆haloalkyl, C₁₋₆ alkoxy optionally substituted with one or more halo, C₁₋₆ alkyl optionally substituted with —OH, or —O—C₃₋₁₀cycloalkyl optionally substituted with one or more halo.

5. The compound of claim 4, or a stereoisomer or tautomer thereof, or a pharmaceutically acceptable salt of any of the foregoing, wherein R^{x} is H, F, or methyl.

6. The compound of claim 4, or a stereoisomer or tautomer thereof, or a pharmaceutically acceptable salt of any of the foregoing, wherein R^{x} is H.

7. The compound of claim 4, or a stereoisomer or tautomer thereof, or a pharmaceutically acceptable salt of any of the foregoing, wherein R^{y} is H, —OH, methoxy, 2-methoxyethoxy, 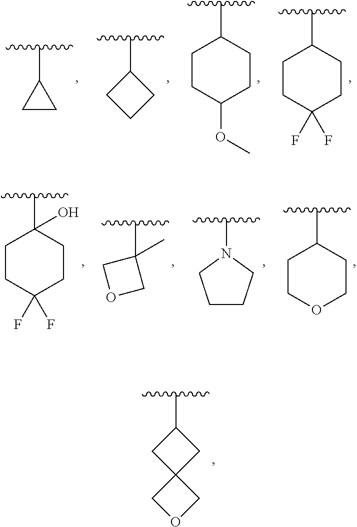
methyl, isopropyl, tert-butyl, difluoromethyl, trifluoromethyl, or 2-hydroxyprop-2-yl.

8. The compound of claim 4, or a stereoisomer or tautomer thereof, or a pharmaceutically acceptable salt of any of the foregoing, wherein R^{y} is H, —OH, C₁₋₆ alkoxy optionally substituted with one or more C₁₋₆ alkoxy, C₃₋₁₀cycloalkyl, or C₁-6alkyl optionally substituted with one or more halo.

9. The compound of claim 4, or a stereoisomer or tautomer thereof, or a pharmaceutically acceptable salt of any of the foregoing, wherein R^{y} is H, —OH, methoxy, 2-methoxyethoxy, methyl, tert-butyl, or difluoromethyl.

10. The compound of claim 4, or a stereoisomer or tautomer thereof, or a pharmaceutically acceptable salt of any of the foregoing, wherein R^{y} is H.

11. The compound of claim 4, or a stereoisomer or tautomer thereof, or a pharmaceutically acceptable salt of any of the foregoing, wherein the compound is selected from the group consisting of compounds 36, 72-75, 144-150, 188-192, 201-220, 222-224, 231-243, 246, 247, 249-266, 268-276, 278, 284, 290, 293, 294, 297, 299, 301, 302, 304-306, 308, 310, 312, 313, 317, 319, 320, 330, 343, 344, 346, 347, 349-351, 354, and 356-358, or a stereoisomer or tautomer thereof, or a pharmaceutically acceptable salt of any of the foregoing.

12. A compound of formula (I-A1): 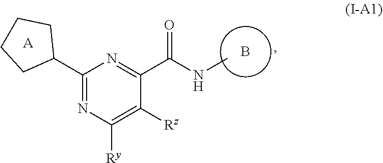
or a stereoisomer or tautomer thereof, or a pharmaceutically acceptable salt of any of the foregoing, wherein:
R^{y} is H, —OH, C₁₋₆alkoxy, C₃₋₁₀cycloalkyl, 3-10 membered heterocyclyl, or C₁₋₆alkyl,
wherein the C₁₋₆ alkoxy of R^{y} is optionally substituted with one or more C₁₋₆alkoxy, the C₃₋₁₀cycloalkyl of R^{y} is optionally substituted with one or more halo, C₁₋₆alkoxy, or —OH, the 3-10 membered heterocyclyl of R^{y} is optionally substituted with one or more C₁₋₆alkyl, and the C₁₋₆ alkyl of R^{y} is optionally substituted with one or more halo or —OH;
R^{z} is H, halo, —NH₂, C₁₋₆ alkoxy, or C₁₋₆ alkyl; 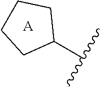
is:
(i) 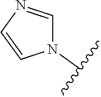 [SMILES: *n1ccnc1]
that is optionally substituted with one or more —C(O)—NH₂, or
(ii) 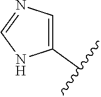 [SMILES: *c1cnc[nH]1]
that is optionally substituted with one or more C₁₋₆ alkyl, or
(iii) 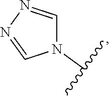 [SMILES: *n1cnnc1]
or
(iv) 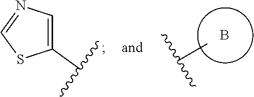
is:
(i) C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆ haloalkyl), phenyl, phenoxy, or pyridinyl,
wherein the C₁₋₆ alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
(ii) 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆ alkoxy, or phenyl, wherein the phenyl of R^{b} is optionally substituted with one or more C₁₋₆ haloalkyl, or
(iii) phenyl, wherein the phenyl is optionally substituted with one or more halo, or with C₁₋₆alkyl optionally substituted with —OH, or
(iv) pyridinyl, wherein the pyridinyl is optionally substituted with one or more halo, C₁₋₆haloalkyl, C₁₋₆ alkoxy optionally substituted with one or more halo, C₁₋₆ alkyl optionally substituted with —OH, or —O—C₃₋₁₀cycloalkyl optionally substituted with one or more halo;
provided that the compound of formula (I-A1) or the stereoisomer or tautomer thereof, or the pharmaceutically acceptable salt of any of the foregoing, is not a compound selected from the compounds of Table 5X or a stereoisomer or tautomer thereof, or a pharmaceutically acceptable salt of any of the foregoing.

13. The compound of claim 12, or a stereoisomer or tautomer thereof, or a pharmaceutically acceptable salt of any of the foregoing, wherein the compound is selected from the group consisting of compounds 67-70, 78, 79, 81-87, 93, 94, 106-131, 137-142, 151-155, 157-165, 167-178, 181-186, 193-200, 227-230, 248, 267, 277, 280-283, 285-289, 291, 292, 295, 300, 307, 309, 311, 315, 316, 321-329, 331-342, 345, 348, 352, 353, and 355, or a stereoisomer or tautomer thereof, or a pharmaceutically acceptable salt of any of the foregoing.

14. A compound of formula (I-A1): 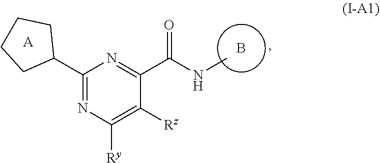
or a stereoisomer or tautomer thereof, or a pharmaceutically acceptable salt of any of the foregoing, wherein:
R^{y} is cyclohexyl or 3-10 membered heterocyclyl,
wherein the cyclohexyl is optionally substituted with one or more halo, C₁₋₆ alkoxy, or —OH, and the 3-10 membered heterocyclyl is optionally substituted with one or more C₁₋₆alkyl;
R^{z} is H, halo, —NH₂, C₁₋₆ alkoxy, or C₁₋₆ alkyl; 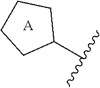
is:
(i) 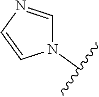 [SMILES: *n1ccnc1]
that is optionally substituted with one or more —C(O)—NH₂, or
(ii) 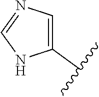 [SMILES: *c1cnc[nH]1]
that is optionally substituted with one or more C₁₋₆ alkyl, or
(iii) 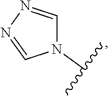 [SMILES: *n1cnnc1]
or
(iv) 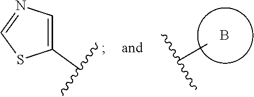
is:
(i) C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆ haloalkyl), phenyl, phenoxy, or pyridinyl,
wherein the C₁₋₆ alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
(ii) 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆ alkoxy, or phenyl, wherein the phenyl of R^{b} is optionally substituted with one or more C₁₋₆ haloalkyl, or
(iii) phenyl, wherein the phenyl is optionally substituted with one or more halo, or with C₁₋₆alkyl optionally substituted with —OH, or
(iv) pyridinyl, wherein the pyridinyl is optionally substituted with one or more halo, C₁₋₆haloalkyl, C₁₋₆ alkoxy optionally substituted with one or more halo, C₁₋₆ alkyl optionally substituted with —OH, or —O—C₃₋₁₀cycloalkyl optionally substituted with one or more halo.

15. The compound of claim 14, or a stereoisomer or tautomer thereof, or a pharmaceutically acceptable salt of any of the foregoing, wherein R^{y} is

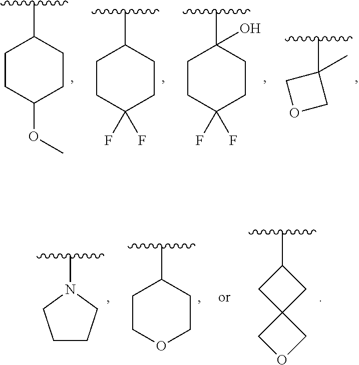

16. The compound of claim 14, or a stereoisomer or tautomer thereof, or a pharmaceutically acceptable salt of any of the foregoing, wherein R^{y} is

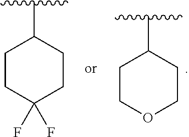 [SMILES: *C1CCC(F)(F)CC1 ; *C1CCOCC1]

17. The compound of claim 14, or a stereoisomer or tautomer thereof, or a pharmaceutically acceptable salt of any of the foregoing, wherein R^{z} is H, F, C₁, —NH₂, methoxy, or methyl.

18. The compound of claim 14, or a stereoisomer or tautomer thereof, or a pharmaceutically acceptable salt of any of the foregoing, wherein R^{z} is H.

19. The compound of claim 14, or a stereoisomer or tautomer thereof, or a pharmaceutically acceptable salt of any of the foregoing, wherein the compound is selected from the group consisting of compounds 193-200, 227-230, 277, 280-283, 285-289, 291, 292, 300, 316, 322, 324, 327, 331, 332, 334, 336, 337, 339, 342, 345, 348, 352, 353, and 355, or a stereoisomer or tautomer thereof, or a pharmaceutically acceptable salt of any of the foregoing.

20. A compound of formula (I-A3): 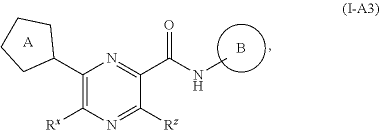
or a stereoisomer or tautomer thereof, or a pharmaceutically acceptable salt of any of the foregoing, wherein:
R^{x} is H, halo, or C₁₋₆alkyl;
R^{z} is H, halo, —NH₂, C₁₋₆ alkoxy, or C₁₋₆ alkyl; 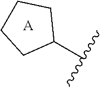
is:
(i) 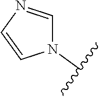 [SMILES: *n1ccnc1]
that is optionally substituted with one or more —C(O)—NH₂, or
(ii) 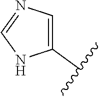 [SMILES: *c1cnc[nH]1]
that is optionally substituted with one or more C₁₋₆ alkyl, or
(iii) 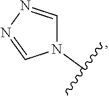 [SMILES: *n1cnnc1]
or
(iv) 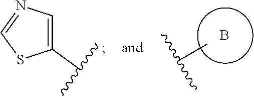
is:
(i) C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆ haloalkyl), phenyl, phenoxy, or pyridinyl,
wherein the C₁₋₆ alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
(ii) 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆ alkoxy, or phenyl, wherein the phenyl of R^{b} is optionally substituted with one or more C₁₋₆ haloalkyl, or
(iii) phenyl, wherein the phenyl is optionally substituted with one or more halo, or with C₁₋₆alkyl optionally substituted with —OH, or
(iv) pyridinyl, wherein the pyridinyl is optionally substituted with one or more halo, C₁₋₆haloalkyl, C₁₋₆ alkoxy optionally substituted with one or more halo, C₁₋₆ alkyl optionally substituted with —OH, or —O—C₃₋₁₀cycloalkyl optionally substituted with one or more halo;
provided that the compound of formula (I-A3) or the stereoisomer or tautomer thereof, or the pharmaceutically acceptable salt of any of the foregoing, is not a compound selected from the compounds of Table 6X, or a stereoisomer or tautomer thereof, or a pharmaceutically acceptable salt of any of the foregoing.

21. The compound of claim 20, or a stereoisomer or tautomer thereof, or a pharmaceutically acceptable salt of any of the foregoing, wherein the compound is selected from the group consisting of compounds 55, 88-92, 102-105, 279, 296, 298, 303, 314, and 318, or a stereoisomer or tautomer thereof, or a pharmaceutically acceptable salt of any of the foregoing.

22. A compound of formula (I-B1): 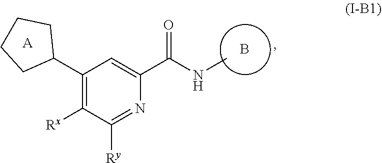
or a stereoisomer or tautomer thereof, or a pharmaceutically acceptable salt of any of the foregoing, wherein:
R^{x} is H, halo, or C₁₋₆alkyl;
R^{y} is H, —OH, C₁₋₆alkoxy, C₃₋₁₀cycloalkyl, 3-10 membered heterocyclyl, or C₁₋₆alkyl,
wherein the C₁₋₆ alkoxy of R^{y} is optionally substituted with one or more C₁₋₆alkoxy, the C₃₋₁₀cycloalkyl of R^{y} is optionally substituted with one or more halo, C₁₋₆alkoxy, or —OH, the 3-10 membered heterocyclyl of R^{y} is optionally substituted with one or more C₁₋₆alkyl, and the C₁₋₆ alkyl of R^{y} is optionally substituted with one or more halo or —OH; 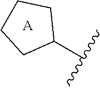
is:
(i) 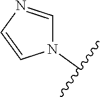 [SMILES: *n1ccnc1]
that is optionally substituted with one or more —C(O)—NH₂, or
(ii) 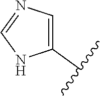 [SMILES: *c1cnc[nH]1]
that is optionally substituted with one or more C₁₋₆ alkyl, or
(iii) 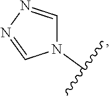 [SMILES: *n1cnnc1]
or
(iv) 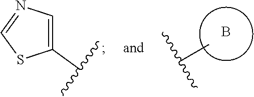
is:
(i) C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆ haloalkyl), phenyl, phenoxy, or pyridinyl,
wherein the C₁₋₆ alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
(ii) 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆ alkoxy, or phenyl, wherein the phenyl of R^{b} is optionally substituted with one or more C₁₋₆ haloalkyl, or
(iii) phenyl, wherein the phenyl is optionally substituted with one or more halo, or with C₁₋₆alkyl optionally substituted with —OH, or
(iv) pyridinyl, wherein the pyridinyl is optionally substituted with one or more halo, C₁₋₆haloalkyl, C₁₋₆ alkoxy optionally substituted with one or more halo, C₁₋₆ alkyl optionally substituted with —OH, or —O—C₃₋₁₀cycloalkyl optionally substituted with one or more halo;
provided that the compound of formula (I-B1) or the stereoisomer or tautomer thereof, or the pharmaceutically acceptable salt of any of the foregoing, is not a compound selected from the compounds of Table 4X, or a stereoisomer or tautomer thereof, or a pharmaceutically acceptable salt of any of the foregoing.

23. The compound of claim 22, or a stereoisomer or tautomer thereof, or a pharmaceutically acceptable salt of any of the foregoing, wherein the compound is selected from the group consisting of compounds 2-7, or a stereoisomer or tautomer thereof, or a pharmaceutically acceptable salt of any of the foregoing.

24. A compound of formula (I-B3): 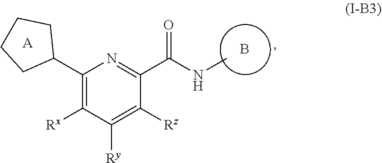
or a stereoisomer or tautomer thereof, or a pharmaceutically acceptable salt of any of the foregoing, wherein:
R^{x} is H, halo, or C₁₋₆alkyl;
R^{y} is H, —OH, C₁₋₆alkoxy, C₃₋₁₀cycloalkyl, 3-10 membered heterocyclyl, or C₁₋₆alkyl,
wherein the C₁₋₆ alkoxy of R^{y} is optionally substituted with one or more C₁₋₆alkoxy, the C₃₋₁₀ cycloalkyl of R^{y} is optionally substituted with one or more halo, C₁₋₆alkoxy, or —OH, the 3-10 membered heterocyclyl of R^{y} is optionally substituted with one or more C₁₋₆alkyl, and the C₁₋₆ alkyl of R^{y} is optionally substituted with one or more halo or —OH;
R^{z} is H, halo, —NH₂, C₁₋₆ alkoxy, or C₁₋₆ alkyl; 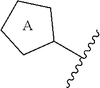
is:
(i) 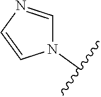 [SMILES: *n1ccnc1]
that is optionally substituted with one or more —C(O)—NH₂, or
(ii) 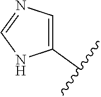 [SMILES: *c1cnc[nH]1]
that is optionally substituted with one or more C₁₋₆ alkyl, or
(iii) 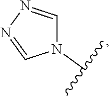 [SMILES: *n1cnnc1]
or
(iv) 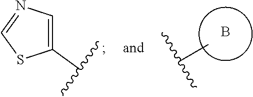
is:
(i) C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆haloalkyl, C₁₋₆alkoxy, —C(O)—C₁₋₆alkoxy, —NH(C₁₋₆ haloalkyl), phenyl, phenoxy, or pyridinyl,
wherein the C₁₋₆ alkoxy of R^{a} is optionally substituted with one or more halo, phenyl, or C₁₋₆ alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl, or
(ii) 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆ alkoxy, or phenyl, wherein the phenyl of R^{b} is optionally substituted with one or more C₁₋₆ haloalkyl, or
(iii) phenyl, wherein the phenyl is optionally substituted with one or more halo, or with C₁₋₆alkyl optionally substituted with —OH, or
(iv) pyridinyl, wherein the pyridinyl is optionally substituted with one or more halo, C₁-6haloalkyl, C₁₋₆ alkoxy optionally substituted with one or more halo, C₁₋₆ alkyl optionally substituted with —OH, or —O—C₃₋₁₀cycloalkyl optionally substituted with one or more halo;
provided that the compound of formula (I-B3) or the stereoisomer or tautomer thereof, or the pharmaceutically acceptable salt of any of the foregoing, is not a compound selected from the compounds of Table 2X, or a stereoisomer or tautomer thereof, or a pharmaceutically acceptable salt of any of the foregoing.

25. The compound of claim 24, or a stereoisomer or tautomer thereof, or a pharmaceutically acceptable salt of any of the foregoing, wherein the compound is selected from the group consisting of compounds 11, 12, 14-19, 22-24, 27, 29, 31, 33-35, 38-49, 51-54, 57, 58, 60-66, 76, 77, 95-101, 132-136, 143, 166, 179, 180, 187, 221, 225, 226, 244, and 245, or a stereoisomer or tautomer thereof, or a pharmaceutically acceptable salt of any of the foregoing.

26. The compound of claim 1, or a stereoisomer or tautomer thereof, or a pharmaceutically acceptable salt of any of the foregoing, wherein 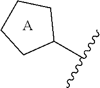
is

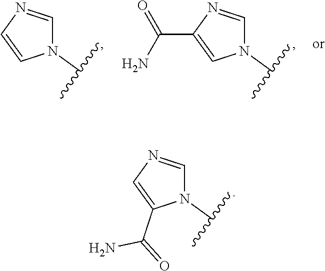 [SMILES: *n1ccnc1 ; *n1cnc(C(N)=O)c1 ; *n1cncc1C(N)=O]

27. The compound of claim 26, or a stereoisomer or tautomer thereof, or a pharmaceutically acceptable salt of any of the foregoing, wherein 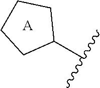
is

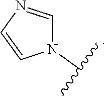 [SMILES: *n1ccnc1]

28. The compound of claim 1, or a stereoisomer or tautomer thereof, or a pharmaceutically acceptable salt of any of the foregoing, wherein 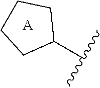
is

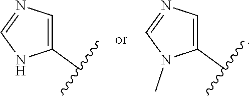

29. The compound of claim 28, or a stereoisomer or tautomer thereof, or a pharmaceutically acceptable salt of any of the foregoing, wherein 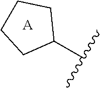
is

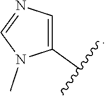 [SMILES: *c1cncn1C]

30. The compound of claim 1, or a stereoisomer or tautomer thereof, or a pharmaceutically acceptable salt of any of the foregoing, wherein 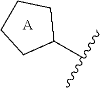
is

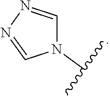 [SMILES: *n1cnnc1]

31. The compound of claim 1, or a stereoisomer or tautomer thereof, or a pharmaceutically acceptable salt of any of the foregoing, wherein 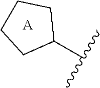
is

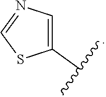 [SMILES: *c1cncs1]

32. The compound of claim 1, or a stereoisomer or tautomer thereof, or a pharmaceutically acceptable salt of any of the foregoing, wherein 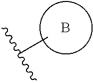
is C₄₋₉ cycloalkyl, wherein the C₄₋₉ cycloalkyl is optionally substituted with one or more R^{a}, wherein each R^{a} is independently —OH, halo, C₁₋₆alkyl, C₁₋₆ haloalkyl, C₁₋₆ alkoxy, —C(O)—C₁₋₆alkoxy, —NH(C₁₋₆ haloalkyl), phenyl, phenoxy, or pyridinyl,
wherein the C₁₋₆ alkoxy of R^{a} is optionally substituted with one or more halo, phenyl, or C₁₋₆alkoxy, the C₁₋₆ alkyl of R^{a} is optionally substituted with one or more —OH or C₁₋₆alkoxy, the phenyl of R^{a} is optionally substituted with one or more halo or C₁₋₆alkoxy, and the pyridinyl of R^{a} is optionally substituted with one or more C₁₋₆haloalkyl.

33. The compound of claim 32, or a stereoisomer or tautomer thereof, or a pharmaceutically acceptable salt of any of the foregoing, wherein 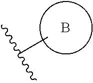
is

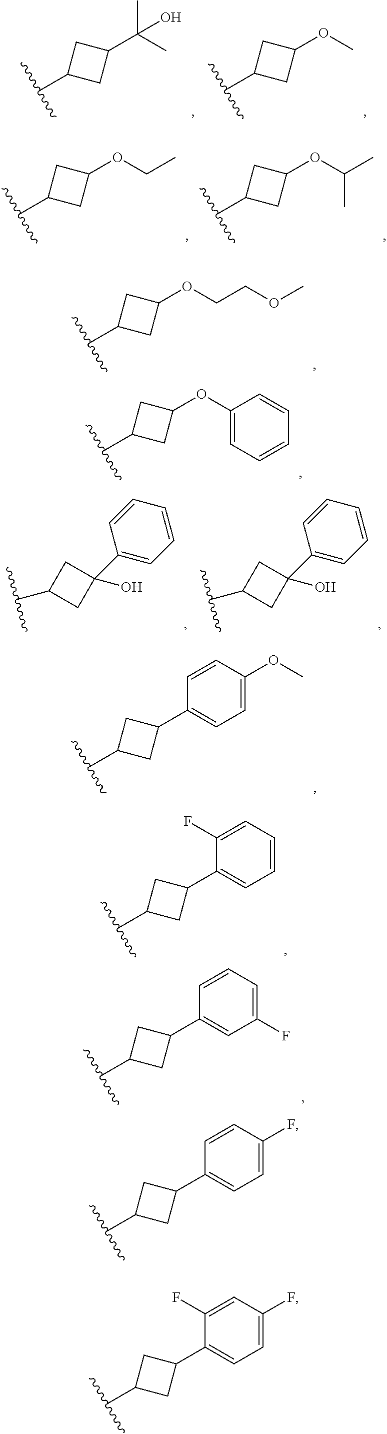

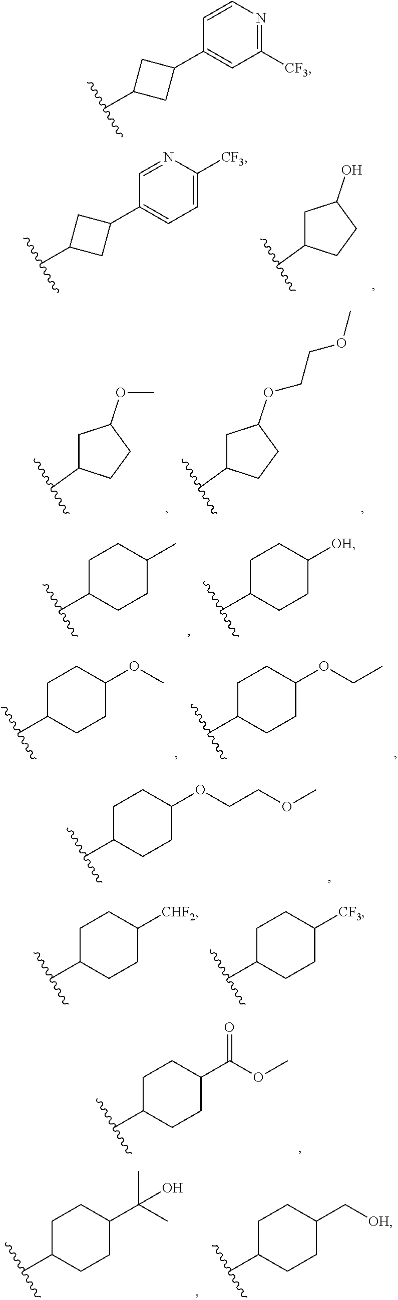

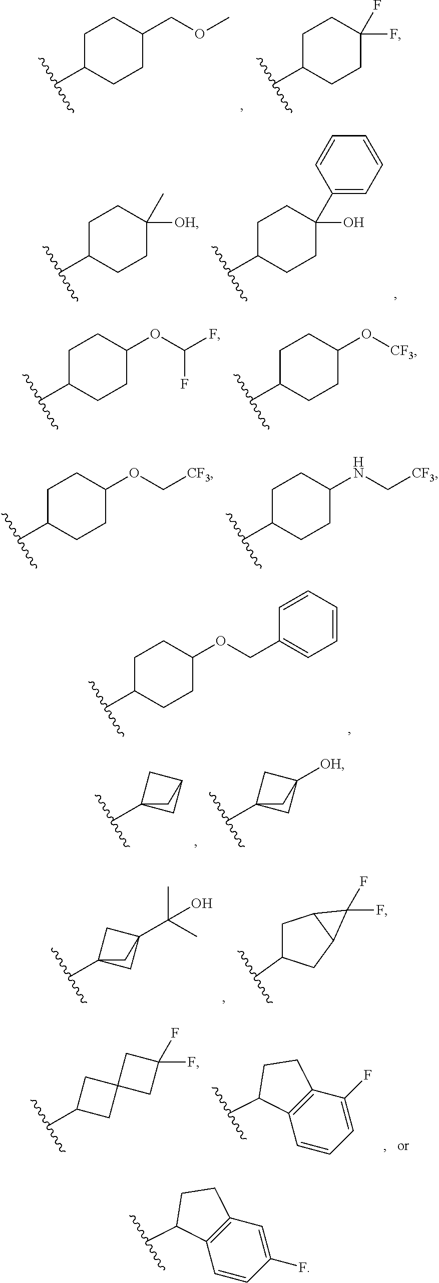

34. The compound of claim 32, or a stereoisomer or tautomer thereof, or a pharmaceutically acceptable salt of any of the foregoing, wherein 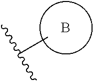
is

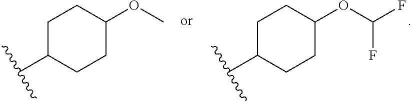

35. The compound of claim 32, or a stereoisomer or tautomer thereof, or a pharmaceutically acceptable salt of any of the foregoing, wherein 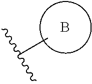
is

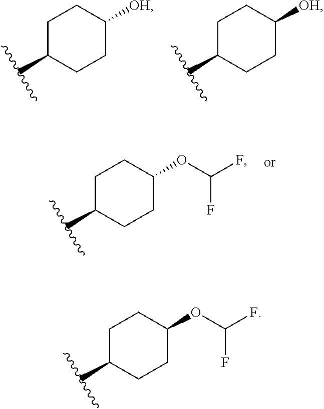 [SMILES: *[C@H]1CC[C@H](O)CC1 ; *[C@H]1CC[C@@H](O)CC1 ; *[C@H]1CC[C@H](OC(F)F)CC1 ; "or" ; *[C@H]1CC[C@@H](OC(F)F)CC1]

36. The compound of claim 32, or a stereoisomer or tautomer thereof, or a pharmaceutically acceptable salt of any of the foregoing, wherein 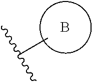
is

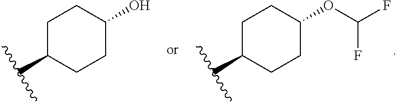 [SMILES: *[C@H]1CC[C@H](O)CC1 ; "or" ; *[C@H]1CC[C@H](OC(F)F)CC1]

37. The compound of claim 32, or a stereoisomer or tautomer thereof, or a pharmaceutically acceptable salt of any of the foregoing, wherein 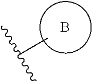
is

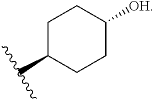 [SMILES: *[C@H]1CC[C@H](O)CC1]

38. The compound of claim 32, or a stereoisomer or tautomer thereof, or a pharmaceutically acceptable salt of any of the foregoing, wherein 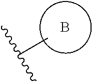
is

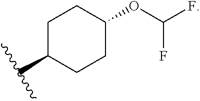 [SMILES: *[C@H]1CC[C@H](OC(F)F)CC1]

39. The compound of claim 1, or a stereoisomer or tautomer thereof, or a pharmaceutically acceptable salt of any of the foregoing, wherein 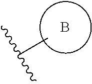
is 4-9 membered heterocyclyl, wherein the 4-9 membered heterocyclyl is optionally substituted with one or more R^{b}, wherein each R^{b} is independently halo, C₁₋₆ alkyl, oxo, —C(O)—C₁₋₆ alkyl, —C(O)—C₁₋₆alkoxy, or phenyl, wherein the phenyl of R^{b} is optionally substituted with one or more C₁₋₆ haloalkyl.

40. The compound of claim 39, or a stereoisomer or tautomer thereof, or a pharmaceutically acceptable salt of any of the foregoing, wherein 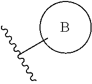
is

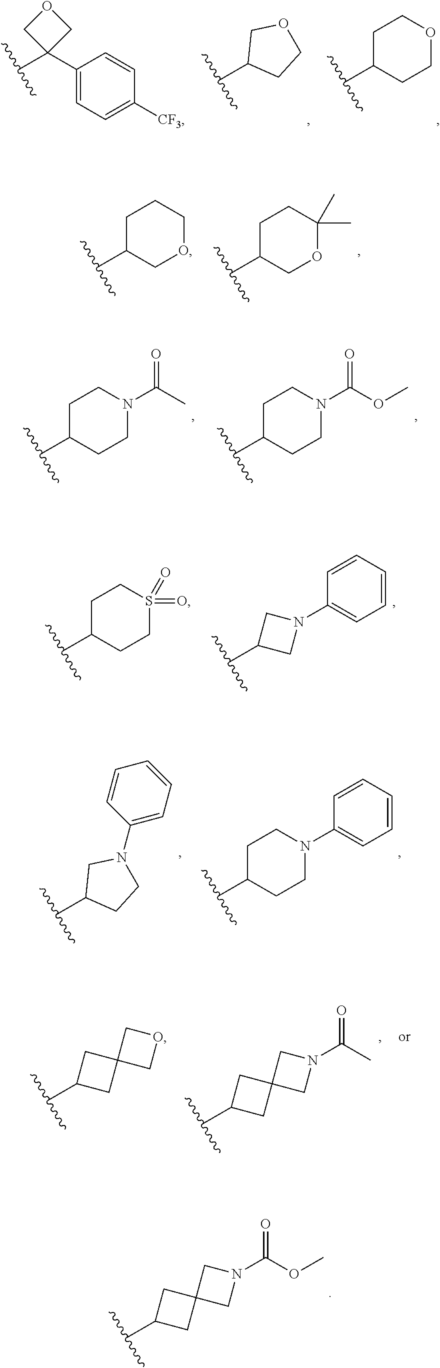

41. The compound of claim 1, or a pharmaceutically acceptable salt thereof, wherein 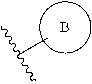
is phenyl, wherein the phenyl is optionally substituted with one or more halo, or with C₁₋₆ alkyl optionally substituted with —OH.

42. The compound of claim 41, or a pharmaceutically acceptable salt thereof, wherein 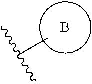
is

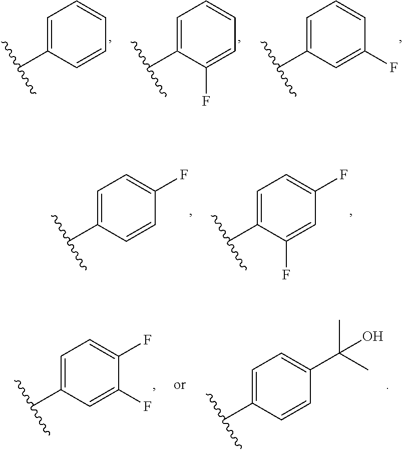

43. The compound of claim 1, or a stereoisomer or tautomer thereof, or a pharmaceutically acceptable salt of any of the foregoing, wherein 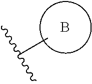
is pyridinyl, wherein the pyridinyl is optionally substituted with one or more halo, C₁₋₆ haloalkyl, C₁₋₆ alkoxy optionally substituted with one or more halo, C₁₋₆ alkyl optionally substituted with —OH, or —O—C₃₋₁₀cycloalkyl optionally substituted with one or more halo.

44. The compound of claim 43, or a pharmaceutically acceptable salt thereof, wherein 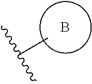
is

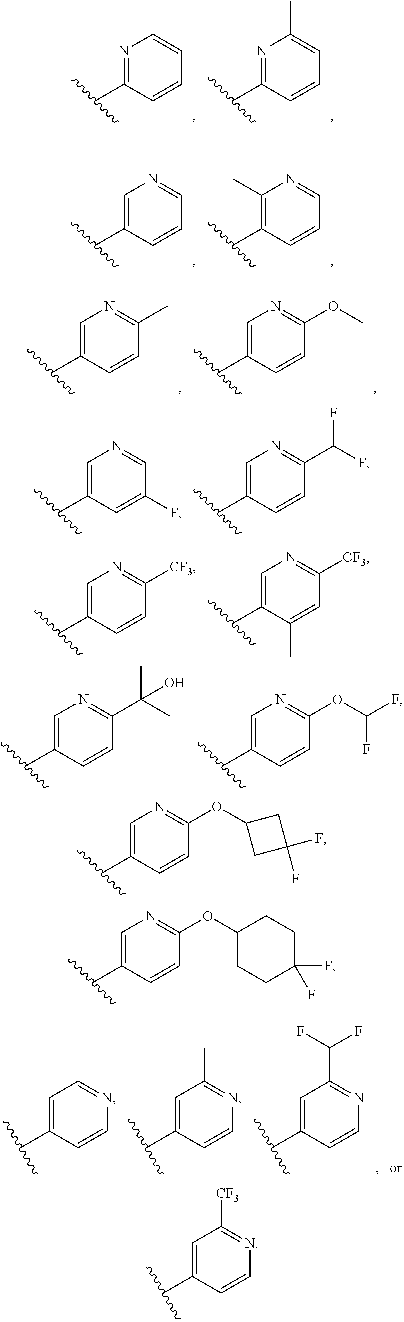

45. The compound of claim 43, or a pharmaceutically acceptable salt thereof, wherein 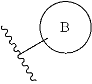
is

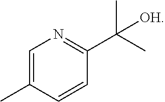

46. A compound selected from the group consisting of compounds 2-7, 9, 11, 12, 14-19, 22-24, 27, 29, 31, 33-36, 38-49, 51-55, 57, 58, 60-70, 72-79, 81-84, 86-110, 112-155, 158-180, and 184-186, or a stereoisomer or tautomer thereof, or a pharmaceutically acceptable salt of any of the foregoing.

47. A compound selected from the group consisting of 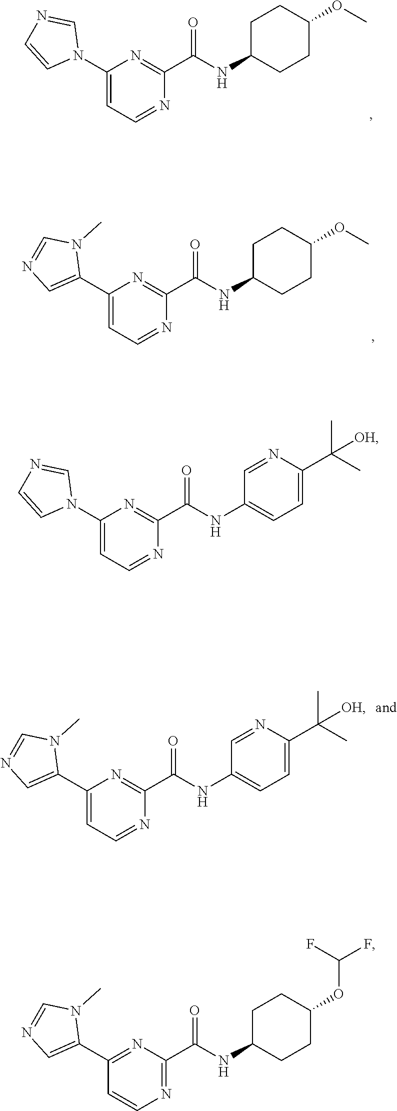 [SMILES: CO[C@H]1CC[C@H](NC(=O)c2nccc(-n3ccnc3)n2)CC1 ; CO[C@H]1CC[C@H](NC(=O)c2nccc(-c3cncn3C)n2)CC1 ; CC(C)(O)c1ccc(NC(=O)c2nccc(-n3ccnc3)n2)cn1 ; Cn1cncc1-c1ccnc(C(=O)Nc2ccc(C(C)(C)O)nc2)n1 ; Cn1cncc1-c1ccnc(C(=O)N[C@H]2CC[C@H](OC(F)F)CC2)n1]
or a stereoisomer or tautomer thereof, or a pharmaceutically acceptable salt of any of the foregoing.

48. A compound selected from the group consisting of 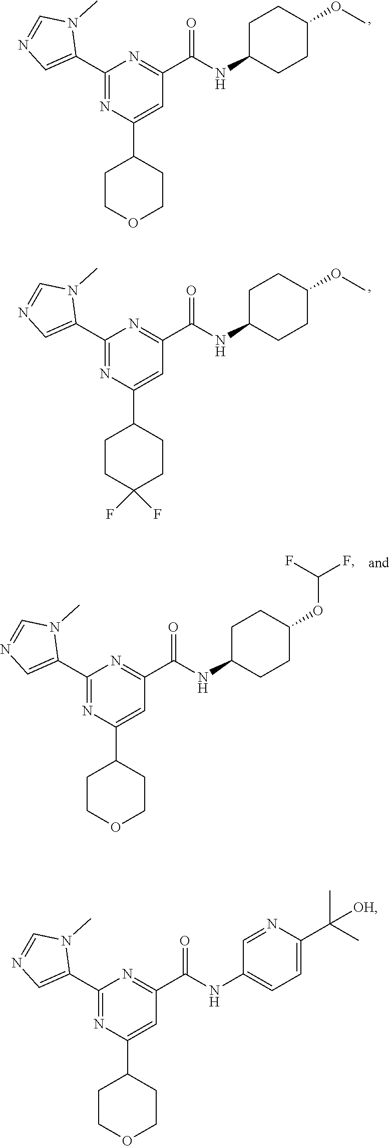 [SMILES: CO[C@H]1CC[C@H](NC(=O)c2cc(C3CCOCC3)nc(-c3cncn3C)n2)CC1 ; CO[C@H]1CC[C@H](NC(=O)c2cc(C3CCC(F)(F)CC3)nc(-c3cncn3C)n2)CC1 ; Cn1cncc1-c1nc(C(=O)N[C@H]2CC[C@H](OC(F)F)CC2)cc(C2CCOCC2)n1 ; "and" ; Cn1cncc1-c1nc(C(=O)Nc2ccc(C(C)(C)O)nc2)cc(C2CCOCC2)n1]
or a stereoisomer or tautomer thereof, or a pharmaceutically acceptable salt of any of the foregoing.

49. A pharmaceutical composition, comprising: (i) an effective amount of a compound of claim 1, or a stereoisomer or tautomer thereof, or a pharmaceutically acceptable salt of any of the foregoing; and (ii) one or more pharmaceutically acceptable excipients.

50. A method of treating a disease, disorder, or condition mediated by CD38 activity in a subject in need thereof, comprising administering to the subject an effective amount of a compound of claim 1, or a stereoisomer or tautomer thereof, or a pharmaceutically acceptable salt of any of the foregoing, or a pharmaceutical composition of claim 49.

51. The method of claim 50, wherein the disease, disorder, or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, a muscle disease, and a muscle wasting disorder.

52. The method of claim 50, wherein the disease, disorder, or condition is selected from the group consisting of obesity, atherosclerosis, insulin resistance, type 2 diabetes, cardiovascular disease, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury.
